(19) United States
(12) Patent Application Publication
(10) Pub. No.: US 2021/0084902 A1
(43) Pub. Date: Mar. 25, 2021

(54) FUNGICIDAL MIXTURE COMPRISING SUBSTITUTED 3-PHENYL-5-(TRIFLUOROMETHYL)-1,2,4-OXADIAZOLES

(71) Applicant: BASF SE, Ludwigshafen am Rhein (DE)

(72) Inventors: Violeta TERTERYAN-SEISER, Ludwigshafen (DE); Maria Angelica QUINTERO PALOMAR, Limburgerhof (DE); Wassilios GRAMMENOS, Ludwigshafen (DE); Christine WIEBE, Ludwigshafen (DE); Dieter STROBEL, Limburgerhof (DE); Paulo Sergio Jose Dos SANTOS, Sao Paulo (BR); Jeffrey Scott BARNES, Research Triangle Park, NC (US)

(21) Appl. No.: 16/610,182

(22) PCT Filed: Apr. 19, 2018
(86) PCT No.: PCT/EP2018/059984

§ 371 Date: Nov. 1, 2019

(30) Foreign Application Priority Data
May 2, 2017 (EP) 17168996.1

(51) Int. Cl. A01N 43/82; A01N 43/80

(52) U.S. Cl. CPC A01N 43/82; A01N 43/80

(57) ABSTRACT

Fungicidal mixture comprising substituted 3-phenyl-5-(trifluoromethyl)-1,2,4-oxadiazoles The present invention relates to fungicidal mixtures comprising at least one substituted 3-phenyl-5-(trifluoromethyl)-1,2,4-oxadiazoles (compounds I) and at least one active compound II in a weight ratio of from 100:1 to 1:100; to a method for controlling phytopathogenic harmful fungi using mixtures of at least one compound I and at least one compound II in a weight ratio of from 100:1 to 1:100; to the use of mixtures comprising compounds I and compounds II for controlling phytopathogenic harmful fungi; to agrochemical compositions comprising these mixtures; and to agrochemical compositions further comprising seed.

The present invention relates to a fungicidal mixture comprising, as active components
1) at least one active compound of the formula I, or an N-oxide, or an agriculturally useful salt thereof,

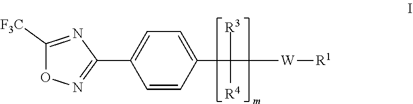

wherein:
W is —(C═O)—NR²-#, —(C═S)—NR²-#, —NR²—(C═O)-# or —NR²—(C═S)-#; wherein # denotes the position, which is attached to the group R¹;
R¹ is C₁-C₆-alkyl, C₃-C₈-cycloalkyl, C₃-C₈-cycloalkenyl, C₂-C₆-alkenyl, C₂-C₆-alkynyl, C₁-C₆-alkoxyimino-C₁-C₄-alkyl or phenyl; wherein the phenyl ring is unsubstituted or substituted with 1, 2, 3 or up to the maximum possible number of identical or different radicals selected from the group consisting of halogen, C₁-C₆-alkyl and C₁-C₆-alkoxy;
R² is hydrogen, C₁-C₆-alkyl, C₂-C₆-alkenyl, C₂-C₆-alkynyl, C₁-C₆-alkoxy, C₃-C₈-cycloalkyl, C₃-C₈-cycloalkenyl, and C₃-C₅-cycloalkyl-C₁-C₄-alkyl; and wherein any of the aliphatic or cyclic groups are unsubstituted or substituted with 1, 2, 3 or up to the maximum possible number of identical or different radicals selected from the group consisting of halogen, hydroxy, oxo, cyano, C₁-C₆-alkyl, C₁-C₆-alkoxy and C₃-C₈-cycloalkyl; or
R¹ and R² together with the nitrogen atom to which they are bound form a saturated or partially unsaturated mono- or bicyclic 3- to 7-membered heterocycle, wherein the heterocycle includes beside one nitrogen atom and one or more carbon atoms no further heteroatom or 1, 2 or 3 further heteroatoms independently selected from N, O and S as ring member atoms; and wherein one or two CH₂ groups of the heterocycle may be replaced by one or two groups independently selected from the group of C(═O) and C(═S); and wherein the heterocycle is unsubstituted or carries 1, 2, 3, 4 or up to the maximum possible number of identical or different radicals selected from the group consisting of halogen, cyano, C₁-C₆-alkyl, C₁-C₆-haloalkyl, C₁-C₆-alkoxy,
m is 0 or 1;
R³, R⁴ independently of each other are selected from the group consisting of hydrogen, halogen, cyano, C₁-C₄-alkyl, C₁-C₄-alkenyl, C₁-C₄-alkynyl, C₁-C₄-haloalkyl and C₁-C₄-alkoxy;
or R³ and R⁴ together with the carbon atom to which they are bound form a cyclopropyl ring;
and as component
2) at least one active compound II selected from groups A) to O), or an N-oxide, or an agriculturally useful salt thereof:

### A) Respiration Inhibitors:

Inhibitors of complex III at Qₒ site: azoxystrobin (A.1.1), coumethoxystrobin (A.1.2), coumoxystrobin (A.1.3), dimoxystrobin (A.1.4), enestroburin (A.1.5), fenaminstrobin (A.1.6), fenoxystrobin/flufenoxystrobin (A.1.7), fluoxastrobin (A.1.8), kresoxim-methyl (A.1.9), mandestrobin (A.1.10), metominostrobin (A.1.11), orysastrobin (A.1.12), picoxystrobin (A.1.13), pyraclostrobin (A.1.14), pyrametostrobin (A.1.15), pyraoxystrobin (A.1.16), trifloxystrobin (A.1.17), 2-(2-(3-(2,6-dichlorophenyl)-1-methyl-allylideneaminooxymethyl)-phenyl)-2-methoxyimino-N-methyl-acetamide (A.1.18), pyribencarb (A.1.19), triclopyricarb/chlorodincarb (A.1.20), famoxadone (A.1.21), fenamidone (A.1.21a), methyl-N-[2-[(1,4-dimethyl-5-phenyl-pyrazol-3-yl)oxylmethyl]phenyl]-N-methoxy-carbamate (A.1.22), 1-[3-chloro-2-[[1-(4-chlorophenyl)-1H-pyrazol-3-yl]oxymethyl]phenyl]-4-methyl-tetrazol-5-one (A.1.23), 1-[3-bromo-2-[[1-(4-chlorophenyl)pyrazol-3-yl]oxymethyl]phenyl]-4-methyl-tetrazol-5-one (A.1.24), 1-[2-[[1-(4-chlorophenyl)pyrazol-3-yl]oxymethyl]-3-methyl-phenyl]-4-methyl-tetrazol-5-one (A.1.25), 1-[2-[[1-(4-chlorophenyl)pyrazol-3-yl]oxymethyl]-3-fluoro-phenyl]-4-methyl-tetrazol-5-one (A.1.26), 1-[2-[[1-(2,4-dichlorophenyl)pyrazol-3-yl]oxymethyl]-3-fluoro-phenyl]-4-methyl-tetrazol-5-one (A.1.27), 1-[2-[[4-(4-chlorophenyl)thiazol-2-yl]oxymethyl]-3-methyl-phenyl]-4-methyl-tetrazol-5-one (A.1.28), 1-[3-chloro-2-[[4-(p-tolyl)thiazol-2-yl]oxymethyl]phenyl]-4-methyl-tetrazol-5-one (A.1.29), 1-[3-cyclopropyl-2-[[2-methyl-4-(1-methylpyrazol-3-yl)phenoxy]-methyl]phenyl]-4-methyl-tetrazol-5-one (A.1.30), 1-[3-(difluoromethoxy)-2-[[2-methyl-4-(1-methylpyrazol-3-yl)phenoxy]methyl]phenyl]-4-methyl-tetrazol-5-one (A.1.31), 1-methyl-4-[3-methyl-2-[[2-methyl-4-(1-methylpyrazol-3-yl)phenoxy]methyl]phenyl]tetrazol-5-one (A.1.32), 1-methyl-4-[3-methyl-2-[[1-[3-(trifluoromethyl)phenyl]-ethylideneamino]oxymethyl]phenyl]tetrazol-5-one (A.1.33), (Z,2E)-5-[1-(2,4-dichlorophenyl)pyrazol-3-yl]-oxy-2-methoxyimino-N,3-dimethyl-pent-3-enamide (A.1.34), (Z,2E)-5-[1-(4-chlorophenyl)pyrazol-3-yl]oxy-2-methoxyimino-N,3-dimethyl-pent-3-enamide (A.1.35), pyriminostrobin (A.1.36), bifujunzhi (A.1.37), 2-(ortho-((2,5-dimethylphenyl-oxymethylen)phenyl)-3-methoxy-acrylic acid methylester (A.1.38);

Inhibitors of complex III at Qᵢ site: cyazofamid (A.2.1), amisulbrom (A.2.2), [(6S,7R,8R)-8-benzyl-3-[(3-hydroxy-4-methoxy-pyridine-2-carbonyl)amino]-6-methyl-4,9-dioxo-1,5-dioxonan-7-yl] 2-methylpropanoate (A.2.3), fenpicoxamid (A.2.4), [(6S,7R,8R)-8-benzyl-3-[[4-methoxy-3-(propanoyloxymethoxy)pyridine-2-carbonyl]amino]-6-methyl-4,9-dioxo-1,5-dioxonan-7-yl] 2-methylpropanoate (A.2.5), florylpicoxamid (A.2.6);

Inhibitors of complex II: benodanil (A.3.1), benzovindiflupyr (A.3.2), bixafen (A.3.3), boscalid (A.3.4), carboxin (A.3.5), fenfuram (A.3.6), fluopyram (A.3.7), flutolanil (A.3.8), fluxapyroxad (A.3.9), furametpyr (A.3.10), isofetamid (A.3.11), isopyrazam (A.3.12), mepronil (A.3.13), oxycarboxin (A.3.14), penflufen (A.3.15), penthiopyrad (A.3.16), pydiflumetofen (A.3.17), N-[2-(3,4-difluorophenyl)phenyl]-3-(trifluoromethyl)pyrazine-2-carboxamide (A.3.18), sedaxane (A.3.19), tecloftalam (A.3.20), thifluzamide (A.3.21), 3-(difluoromethyl)-1-methyl-N-(1,1,3-trimethylindan-4-yl)pyrazole-4-carboxamide (A.3.22), 3-(trifluoromethyl)-1-methyl-N-(1,1,3-trimethylindan-4-yl)pyrazole-4-carboxamide (A.3.23), 1,3-dimethyl-N-(1,1,3-trimethylindan-4-yl)pyrazole-4-carboxamide (A.3.24), 3-(trifluoromethyl)-1,5-dimethyl-N-(1,1,3-trimethylindan-4-yl)pyrazole-4-carboxamide (A.3.25), 1,3,5-trimethyl-N-(1,1,3-trimethylindan-4-yl)pyrazole-4-carboxamide (A.3.26), 3-(difluoromethyl)-1,5-dimethyl-N-(1,1,3-trimethylindan-4-yl)pyrazole-4-carboxamide (A.3.27), 3-(difluoromethyl)-N-(7-fluoro-1,1,3-trimethyl-indan-4-yl)-1-methyl-pyrazole-4-carboxamide (A.3.28), N-[2-[2-chloro-4-(trifluoromethyl)phenoxy]phenyl]-3-(difluoromethyl)-5-fluoro-1-methyl-pyrazole-4-carboxamide (A.3.29), methyl (E)-2-[2-[(5-cyano-2-methyl-phenoxy)methyl]phenyl]-3-methoxy-prop-2-enoate (A.3.30), N-[(5-chloro-2-isopropyl-phenyl)methyl]-N-cyclopropyl-3-(difluoromethyl)-5 fluoro-1-methyl-pyrazole-4-carboxamide (A.3.31), 2-(difluoromethyl)-N-(1,1,3-trimethyl-indan-4-yl)pyridine-3-carboxamide (A.3.32), 2-(difluoromethyl)-N-[(3R)-1,1,3-trimethylindan-4-yl]pyridine-3-carboxamide (A.3.33), 2-(difluoromethyl)-N-[(3S)3-ethyl-1,1-dimethyl-indan-4-yl]pyridine-3-carboxamide (A.3.34), 2-(difluoromethyl)-N-[(3R)-3-ethyl-1,1-dimethyl-indan-4-yl]pyridine-3-carboxamide (A.3.35), 2-(difluoromethyl)-N-(1,1-dimethyl-3-propyl-indan-4-yl)pyridine-3-carboxamide (A.3.36), 2-(difluoromethyl)-N-[(3R)-1,1-dimethyl-3-propyl-indan-4-yl]pyridine-3-carboxamide (A.3.37), 2-(difluoromethyl)-N-(3-isobutyl-1,1-dimethyl-indan-4-yl)pyridine-3-carboxamide (A.3.38), 2-(difluoromethyl)-N-[(3R)-3-isobutyl-1,1-dimethyl-indan-4 yl]pyridine-3-carboxamide (A.3.39);

Other respiration inhibitors: diflumetorim (A.4.1); nitrophenyl derivates: binapacryl (A.4.2), dinobuton (A.4.3), dinocap (A.4.4), fluazinam (A.4.5), meptyldinocap (A.4.6), ferimzone (A.4.7); organometal compounds: fentin salts, e. g. fentin-acetate (A.4.8), fentin chloride (A.4.9) or fentin hydroxide (A.4.10); ametoctradin (A.4.11); silthiofam (A.4.12);

### B) Sterol Biosynthesis Inhibitors (SBI Fungicides)

C14 demethylase inhibitors: triazoles: azaconazole (B.1.1), bitertanol (B.1.2), bromuconazole (B.1.3), cyproconazole (B.1.4), difenoconazole (B.1.5), diniconazole (B.1.6), diniconazole-M (B.1.7), epoxiconazole (B.1.8), fenbuconazole (B.1.9), fluquinconazole (B.1.10), flusilazole (B.1.11), flutriafol (B.1.12), hexaconazole (B.1.13), imibenconazole (B.1.14), ipconazole (B.1.15), metconazole (B.1.17), myclobutanil (B.1.18), oxpoconazole (B.1.19), paclobutrazole (B.1.20), penconazole (B.1.21), propiconazole (B.1.22), prothioconazole (B.1.23), simeconazole (B.1.24), tebuconazole (B.1.25), tetraconazole (B.1.26), triadimefon (B.1.27), triadimenol (B.1.28), triticonazole (B.1.29), uniconazole (B.1.30), 1-[re(2,3R)-3-(2-chloro-phenyl)-2-(2,4-difluorophenyl)-oxiranylmethyl]-5-thiocyanato-1H-[1,2,4]triazole (B.1.31), 2-[rel (2S,3R)-3-(2-chlorophenyl)-2-(2,4-difluorophenyl)-oxiranylmethyl]-2H-[1,2,4]triazole-3-thiol (B.1.32), 2-[2-chloro-4-(4-chlorophenoxy)phenyl]-1-(1,2,4-triazol-1-yl)pentan-2-ol (B.1.33), 1-[4-(4-chlorophenoxy)-2-(trifluoromethyl)phenyl]-1-cyclopropyl-2-(1,2,4-triazol-1-yl)ethanol (B.1.34), 2-[4-(4-chlorophenoxy)-2-(trifluoromethyl)phenyl]-1-(1,2,4-triazol-1-yl)butan-2-ol (B.1.35), 2-[2-chloro-4-(4-chlorophenoxy)phenyl]-1-(1,2,4-triazol-1-yl)butan-2-ol (B.1.36), ipfentrifluconazole (B.1.37), mefentrifluconazole (B.1.38), 2-[2-chloro-4-(4-chlorophenoxy)phenyl]-3-methyl-1-(1,2,4-triazol-1-yl)butan-2-ol (B.1.39), 2-[4-(4-chlorophenoxy)-2-(trifluoromethyl)phenyl]-1-(1,2,4-triazol-1-yl)pentan-2-ol (B.1.40), 2-[4-(4-fluorophenoxy)-2-(trifluoromethyl)phenyl]-1-(1,2,4-triazol-1-yl)propan-2-ol (B.1.41), 2-[2-chloro-4-(4-chlorophenoxy)phenyl]-1-(1,2,4-triazol-1-yl)pent-3-yn-2-ol (B.1.42), 2-(chloromethyl)-2-methyl-5-(p-tolylmethyl)-1-(1,2,4-triazol-1-ylmethyl)cyclopentanol (B.1.43); imidazoles: imazalil (B.1.44), pefurazoate (B.1.45), prochloraz (B.1.46), triflumizol (B.1.47); pyrimidines, pyridines and piperazines: fenarimol (B.1.49), pyrifenox (B.1.50), triforine (B.1.51), [3-(4-chloro-2-fluoro-phenyl)-5-(2,4-difluorophenyl)isoxazol-4-yl]-(3-pyridyl)methanol (B.1.52);

Delta14-reductase inhibitors: aldimorph (B.2.1), dodemorph (B.2.2), dodemorph-acetate (B.2.3), fenpropimorph (B.2.4), tridemorph (B.2.5), fenpropidin (B.2.6), piperalin (B.2.7), spiroxamine (B.2.8);

Inhibitors of 3-keto reductase: fenhexamid (B.3.1);

Other Sterol biosynthesis inhibitors: chlorphenomizole (B.4.1);

### C) Nucleic Acid Synthesis Inhibitors

Phenylamides or acyl amino acid fungicides: benalaxyl (C.1.1), benalaxyl-M (C.1.2), kiralaxyl (C.1.3), metalaxyl (C.1.4), metalaxyl-M (C.1.5), ofurace (C.1.6), oxadixyl (C.1.7);

Other nucleic acid synthesis inhibitors: hymexazole (C.2.1), octhilinone (C.2.2), oxolinic acid (C.2.3), bupirimate (C.2.4), 5-fluorocytosine (C.2.5), 5-fluoro-2-(p-tolylmethoxy)pyrimidin-4-amine (C.2.6), 5-fluoro-2-(4-fluorophenylmethoxy)pyrimidin-4-amine (C.2.7), 5-fluoro-2-(4-chlorophenylmethoxy)pyrimidin-4 amine (C.2.8);

### D) Inhibitors of Cell Division and Cytoskeleton

Tubulin inhibitors: benomyl (D.1.1), carbendazim (D.1.2), fuberidazole (D1.3), thiabendazole (D.1.4), thiophanate-methyl (D.1.5), 3-chloro-4-(2,6-difluorophenyl)-6-methyl-5-phenyl-pyridazine (D.1.6), 3-chloro-6-methyl-5-phenyl-4-(2,4,6-trifluorophenyl)pyridazine (D.1.7), N-ethyl-2-[(3-ethynyl-8-methyl-6-quinolyl)oxy]butanamide (D.1.8), N-ethyl-2-[(3-ethynyl-8-methyl-6-quinolyl)oxy]-2-methylsulfanyl-acetamide (D.1.9), 2-[(3-ethynyl-8-methyl-6-quinolyl)oxy]-N-(2-fluoroethyl)butanamide (D.1.10), 2-[(3-ethynyl-8-methyl-6-quinolyl)oxy]-N-(2-fluoroethyl)-2-methoxy-acetamide (D.1.11), 2-[(3-ethynyl-8-methyl-6-quinolyl)oxy]-N-propyl-butanamide (D.1.12), 2-[(3-ethynyl-8-methyl-6-quinolyl)oxy]-2-methoxy-N-propyl-acetamide (D.1.13), 2-[(3-ethynyl-8-methyl-6-quinolyl)oxy]-2-methylsulfanyl-N-propyl-acetamide (D.1.14), 2-[(3-ethynyl-8-methyl-6-quinolyl)oxy]-N-(2-fluoroethyl)-2-methylsulfanyl-acetamide (D.1.15), 4-(2-bromo-4-fluoro-phenyl)-N-(2-chloro-6-fluoro-phenyl)-2,5-dimethyl-pyrazol-3-amine (D.1.16);

Other cell division inhibitors: diethofencarb (D.2.1), ethaboxam (D.2.2), pencycuron (D.2.3), fluopicolide (D.2.4), zoxamide (D.2.5), metrafenone (D.2.6), pyriofenone (D.2.7);

### E) Inhibitors of Amino Acid and Protein Synthesis

Methionine synthesis inhibitors: cyprodinil (E.1.1), mepanipyrim (E.1.2), pyrimethanil (E.1.3);

Protein synthesis inhibitors: blasticidin-S(E.2.1), kasugamycin (E.2.2), kasugamycin hydrochloride-hydrate (E.2.3), mildiomycin (E.2.4), streptomycin (E.2.5), oxytetracyclin (E.2.6);

### F) Signal Transduction Inhibitors

MAP/histidine kinase inhibitors: fluoroimid (F.1.1), iprodione (F.1.2), procymidone (F.1.3), vinclozolin (F.1.4), fludioxonil (F.1.5);

G protein inhibitors: quinoxyfen (F.2.1);

### G) Lipid and Membrane Synthesis Inhibitors

Phospholipid biosynthesis inhibitors: edifenphos (G.1.1), iprobenfos (G.1.2), pyrazophos (G.1.3), isoprothiolane (G.1.4);

Lipid peroxidation: dicloran (G.2.1), quintozene (G.2.2), tecnazene (G.2.3), tolclofos-methyl (G.2.4), biphenyl (G.2.5), chloroneb (G.2.6), etridiazole (G.2.7);

Phospholipid biosynthesis and cell wall deposition: dimethomorph (G.3.1), flumorph (G.3.2), mandipropamid (G.3.3), pyrimorph (G.3.4), benthiavalicarb (G.3.5), iprovalicarb (G.3.6), valifenalate (G.3.7);

Compounds affecting cell membrane permeability and fatty acides: propamocarb (G.4.1);

Inhibitors of oxysterol binding protein: oxathiapiprolin (G.5.1), 2-{3-[2-(1-{[3,5-bis(difluoro-methyl-1H-pyrazol-1-yl]acetyl}piperidin-4-yl)-1,3-thiazol-4-yl]-4,5-dihydro-1,2-oxazol-5-yl}-phenyl methanesulfonate (G.5.2), 2-{3-[2-(1-{[3,5-bis(difluoromethyl)-1H-pyrazol-1-yl]-acetyl}piperidin-4-yl) 1,3-thiazol-4-yl]-4,5-dihydro-1,2-oxazol-5-yl}-3-chlorophenyl methane-sulfonate (G.5.3), 4-[1-[2-[3-(difluoromethyl)-5-methyl-pyrazol-1-yl]acetyl]-4-piperidyl]-N-tetralin-1-yl-pyridine-2-carboxamide (G.5.4), 4-[1-[2-[3,5-bis(difluoromethyl)pyrazol-1-yl]acetyl]-4-piperidyl]-N-tetralin-1-yl-pyridine-2-carboxamide (G.5.5), 4-[1-[2-[3-(difluoromethyl)-5-(tri-fluoromethyl)pyrazol-1-yl]acetyl]-4-piperidyl]-N-tetralin-1-yl-pyridine-2-carboxamide (G.5.6), 4-[1-[2-[5-cyclopropyl-3-(difluoromethyl)pyrazol-1-yl]acetyl]-4-piperidyl]-N-tetralin-1-yl-pyridine-2-carboxamide (G.5.7), 4-[1-[2-[5-methyl-3-(trifluoromethyl)pyrazol-1-yl]acetyl]-4-piperidyl]-N-tetralin-1-yl-pyridine-2-carboxamide (G.5.8), 4-[1-[2-[5-(difluoromethyl)-3-(trifluoro-methyl)pyrazol-1-yl]acetyl]-4-piperidyl]-N-tetralin-1-yl-pyridine-2-carboxamide (G.5.9), 4-[1-[2-[3,5-bis(trifluoromethyl)pyrazol-1-yl]acetyl]-4-piperidyl]-N-tetralin-1-yl-pyridine-2-carboxamide (G.5.10), (4-[1-[2-[5-cyclopropyl-3-(trifluoromethyl)pyrazol-1-yl]acetyl]-4-piperidyl]-N-tetralin-1-yl-pyridine-2-carboxamide (G.5.11);

H) Inhibitors with Multi Site Action

Inorganic active substances: Bordeaux mixture (H.1.1), copper (H.1.2), copper acetate (H.1.3), copper hydroxide (H.1.4), copper oxychloride (H.1.5), basic copper sulfate (H.1.6), sulfur (H.1.7);

Thio- and dithiocarbamates: ferbam (H.2.1), mancozeb (H.2.2), maneb (H.2.3), metam (H.2.4), metiram (H.2.5), propineb (H.2.6), thiram (H.2.7), zineb (H.2.8), ziram (H.2.9);

Organochlorine compounds: anilazine (H.3.1), chlorothalonil (H.3.2), captafol (H.3.3), captan (H.3.4), folpet (H.3.5), dichlofluanid (H.3.6), dichlorophen (H.3.7), hexachlorobenzene (H.3.8), pentachlorphenole (H.3.9) and its salts, phthalide (H.3.10), tolylfluanid (H.3.11);

Guanidines and others: guanidine (H.4.1), dodine (H.4.2), dodine free base (H.4.3), guazatine (H.4.4), guazatine-acetate (H.4.5), iminoctadine (H.4.6), iminoctadine-triacetate (H.4.7), iminoctadine-tris(albesilate) (H.4.8), dithianon (H.4.9), 2,6-dimethyl-1H,5H-[1,4]dithiino[2,3-c:5,6-c′]dipyrrole-1,3,5,7(2H,6H)-tetraone (H.4.10);

### I) Cell Wall Synthesis Inhibitors

Inhibitors of glucan synthesis: validamycin (I.1.1), polyoxin B (I.1.2);

Melanin synthesis inhibitors: pyroquilon (I.2.1), tricyclazole (I.2.2), carpropamid (I.2.3), dicyclomet (I.2.4), fenoxanil (I.2.5);

### J) Plant Defence Inducers

Acibenzolar-S-methyl (J.1.1), probenazole (J.1.2), isotianil (J.1.3), tiadinil (J.1.4), prohexadione-calcium (J.1.5); phosphonates: fosetyl (J.1.6), fosetyl-aluminum (J.1.7), phosphorous acid and its salts (J.1.8), potassium or sodium bicarbonate (J.1.9), 4-cyclopropyl-N-(2,4-dimethoxyphenyl)thiadiazole-5-carboxamide (J.1.10), calcium phosphonate (J.1.11), potassium phosphonate (J.1.12);

### K) Unknown Mode of Action

Bronopol (K.1.1), chinomethionat (K.1.2), cyflufenamid (K.1.3), cymoxanil (K.1.4), dazomet (K.1.5), debacarb (K.1.6), diclocymet (K.1.7), diclomezine (K.1.8), difenzoquat (K.1.9), difenzoquat-methylsulfate (K.1.10), diphenylamin (K.1.11), fenitropan (K.1.12), fenpyrazamine (K.1.13), flumetover (K.1.14), flusulfamide (K.1.15), flutianil (K.1.16), harpin (K.1.17), methasulfocarb (K.1.18), nitrapyrin (K.1.19), nitrothal-isopropyl (K.1.20), tolprocarb (K.1.21), oxincopper (K.1.22), proquinazid (K.1.23), tebufloquin (K.1.24), tecloftalam (K.1.25), triazoxide (K.1.26), N′-(4-(4-chloro-3-trifluoromethyl-phenoxy)-2,5-dimethyl-phenyl)-N-ethyl-N-methyl formamidine (K.1.27), N′-(4-(4-fluoro-3-trifluoromethyl-phenoxy)-2,5-dimethyl-phenyl)-N-ethyl-N-methyl formamidine (K.1.28), N′-[4-[[3-[(4-chorophenyl)methyl]-1,2,4-thiadiazo-5-yl]oxy]-2,5-dimethyl-phenyl]-N-ethyl-N-methyl-formamidine (K.1.29), N′-(5-bromo-6-indan-2-yloxy-2-methyl-3-pyridyl)-N-ethyl-N-methyl-formamidine (K.1.30), N′-[5-bromo-6-[1-(3,5-difluorophenyl)ethoxy]-2-methyl-3-pyridyl]-N-ethyl-N-methyl-formamidine (K.1.31), N′-[5-bromo-6-(4-isopropylcyclohexoxy)-2-methyl-3-pyridyl]-N-ethyl-N-methyl-formamidine (K.1.32), N′-[5-bromo-2-methyl-6-(1-phenylethoxy)-3-pyridyl]-N-ethyl-N-methyl-formamidine (K.1.33), N′-(2-methyl-5-trifluoromethyl-4-(3-trimethylsilanyl-propoxy)-phenyl)-N-ethyl-N-methyl formamidine (K.1.34), N′-(5-difluoromethyl-2-methyl-4-(3-trimethylsianyl-propoxy)-phenyl)-N-ethyl-N-methyl formamidine (K.1.35), 2-(4-chloro-phenyl)-N-[4-(3,4-dimethoxy-phenyl)-isoxazol-5-yl]-2-prop-2-ynyloxy-acetamide (K.1.36), 3-[5-(4-chloro-phenyl)-2,3-dimethyl-isoxazolidin-3-yl]-pyridine (pyrisoxazole) (K.1.37), 3-[5-(4-methylphenyl)-2,3-dimethyl-isoxazolidin-3 yl]-pyridine (K.1.38), 5-chloro-1-(4,6-dimethoxy-pyrimidin-2-yl)-2-methyl-1H-benzoimidazole (K.1.39), ethyl (Z)-3-amino-2-cyano-3-phenyl-prop-2-enoate (K.1.40), picarbutrazox (K.1.41), pentyl N-[6-[[(Z)-[(1-methyltetrazol-5-yl)-phenyl-methylene]amino]oxymethyl]-2-pyridyl]carba-mate (K.1.42), but-3-ynyl N-[6-[[(Z)-[(1-methyltetrazol-5-yl)-phenyl-methylene]amino]oxymethyl]-2-pyridyl]carbamate (K.1.43), 2-[2-[(7,8-difluoro-2-methyl-3-quinolyl)oxy]-6-fluoro-phenyl]propan-2-ol (K.1.44), 2-[2-fluoro-6-[(8-fluoro-2-methyl-3-quinolyl)oxy]phen-yl]propan-2-ol (K.1.45), 3-(5-fluoro-3,3,4,4-tetramethyl-3,4-dihydroisoquinolin-1-yl)quinoline (K.1.46), quinofumelin (K.1.47), 3-(4,4,5-trifluoro-3,3-dimethyl-3,4-dihydroisoquinolin-1-yl)quinoline (K.1.48), 9-fluoro-2,2-dimethyl-5-(3-quinolyl)-3H-1,4-benzoxazepine (K.1.49), 2-(6-benzyl-2-pyridyl)quinazoline (K.1.50), 2-[6-(3-fluoro-4-methoxy-phenyl)-5-methyl-2-pyridyl]quinazoline (K.1.51), dichlobentiazox (K.1.52), N′-(2,5-dimethyl-4-phenoxy-phenyl)-N-ethyl-N-methyl-formamidine (K.1.53), N′-[5-bromo-2-methyl-6-(1-methyl-2-propoxy-ethoxy)-3-pyridyl]-N-ethyl-N-methyl-formamidine (K.1.54);

### M) Growth Regulators

abscisic acid (M.1.1), amidochlor, ancymidol, 6-benzylaminopurine, brassinolide, butralin, chlormequat, chlormequat chloride, choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat, mepiquat chloride, naphthaleneacetic acid, N-6-benzyladenine, paclobutrazol, prohexadione, prohexadione-calcium, prohydrojasmon, thidiazuron, triapenthenol, tributyl phosphoro-trithioate, 2,3,5-tri-iodobenzoic acid, trinexapac-ethyl and uniconazole;

N) Herbicides from Classes N.1 to N.15

N.1 Lipid biosynthesis inhibitors: alloxydim (N.1.1), alloxydim-sodium (N.1.2), butroxydim (N.1.3), clethodim (N.1.4), clodinafop (N.1.5), clodinafop-propargyl (N.1.6), cycloxydim (N.1.7), cyhalofop (N.1.8), cyhalofop-butyl (N.1.9), diclofop(N.1.10), diclofop-methyl (N.1.11), fenoxaprop (N.1.12), fenoxaprop-ethyl (N.1.13), fenoxaprop-P (N.1.14), fenoxaprop-P-ethyl (N.1.15), fluazifop (N.1.16), fluazifop-butyl (N.1.17), fluazifop-P (N.1.18), fluazifop-P-butyl (N.1.19), haloxyfop (N.1.20), haloxyfop-methyl (N.1.21), haloxyfop-P (N.1.22), haloxyfop-P-methyl (N.1.23), metamifop (N.1.24), pinoxaden (N.1.25), profoxydim (N.1.26), propaquizafop (N.1.27), quizalofop (N.1.28), quizalofop-ethyl (N.1.29), quizalofop-tefuryl (N.1.30), quizalofop-P (N.1.31), quizalofop-P-ethyl (N.1.32), quizalofop-P-tefuryl (N.1.33), sethoxydim (N.1.34), tepraloxydim (N.1.35), tralkoxydim (N.1.36), 4-(4′-chloro-4-cyclo¬propyl-2′-fluoro[1,1′-biphenyl]-3-yl)-5-hydroxy-2,2,6,6-tetramethyl-2H-pyran-3(6H)-one ((N.1.37) CAS 1312337-72-6); 4-(2′,4′-dichloro-4-cyclopropyl[1,1′-biphenyl]-3-yl)-5-hydroxy-2,2,6,6-tetramethyl-2H-pyran-3(6H)-one ((N.1.38) CAS 1312337-45-3); 4-(4′-chloro-4-ethyl-2′-fluoro[1,1′-biphenyl]-3-yl)-5-hydroxy-2,2,6,6-tetramethyl-2H-pyran-3(6H)-one ((N.1.39) CAS 1033757-93-5); 4-(2′,4′-Dichloro-4-ethyl[1,1′-biphenyl]-3-yl)-2,2,6,6-tetramethyl-2H-pyran-3,5(4H,6H)-dione ((N.1.40) CAS 1312340-84-3); 5-(acetyloxy)-4-(4′-chloro-4-cyclopropyl-2′-fluoro[1,1′-biphenyl]-3-yl)-3,6-dihydro-2,2,6,6-tetramethyl-2H-pyran-3-one ((N.1.41) CAS 1312337-48-6); 5-(acetyloxy)-4-(2′,4′-dichloro-4-cyclopropyl-[1,1′-biphenyl]-3-yl)-3,6-dihydro-2,2,6,6-tetramethyl-2H-pyran-3-one (N.1.42); 5-(acetyloxy)-4-(4′-chloro-4-ethyl-2′-fluoro[1,1′-biphenyl]-3-yl)-3,6-dihydro-2,2,6,6-tetramethyl-2H-pyran-3-one ((N.1.43) CAS 1312340-82-1); 5-(acetyloxy)-4-(2′,4′-dichloro-4-ethyl[1,1′-biphenyl]-3-yl)-3,6-dihydro-2,2,6,6-tetramethyl-2H-pyran-3-one ((N.1.44) CAS 1033760-55-2); 4-(4′-chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester ((N.1.45) CAS 1312337-51-1); 4-(2′,4′-dichloro-4-cyclopropyl-[1,1′-biphenyl]-3-yl)-5,6-dihydro-2,2,6,6-tetramethyl-5-oxo-2H-pyran-3-yl carbonic acid methyl ester (N.1.46); 4-(4′-chloro-4-ethyl-2′-fluoro[1,1′-biphenyl]-3-yl)-5,6-dihydro-2,2,6,6-tetramethyl-5-oxo-2H-pyran-3-yl carbonic acid methyl ester ((N.1.47) CAS 1312340-83-2); 4-(2′,4′-dichloro-4-ethyl,[1,1′-biphenyl]-3-yl)-5,6-dihydro-2,2,6,6-tetramethyl-5-oxo-2H-pyran-3-yl carbonic acid methyl ester ((N.1.48) CAS 1033760-58-5); benfuresate (N.1.49), butylate (N.1.50), cycloate (N.1.51), dalapon (N.1.52), dimepiperate (N.1.53), EPTC (N.1.54), esprocarb (N.1.55), ethofumesate (N.1.56), flupropanate (N.1.57), molinate (N.1.58), orbencarb (N.1.59), pebulate (N.1.60), prosulfocarb (N.1.61), TCA (N.1.62), thiobencarb (N.1.63), tiocarbazil (N.1.64), triallate (N.1.65) and vernolate (N.1.66);

N.2 ALS inhibitors: amidosulfuron (N.2.1), azimsulfuron (N.2.2), bensulfuron (N.2.3), bensulfuron-methyl (N.2.4), chlorimuron (N.2.5), chlorimuron-ethyl (N.2.6), chlorsulfuron (N.2.7), cinosulfuron (N.2.8), cyclosulfamuron (N.2.9), ethametsulfuron (N.2.10), ethametsulfuron-methyl (N.2.11), ethoxysulfuron (N.2.12), flazasulfuron (N.2.13), flucetosulfuron (N.2.14), flupyrsulfuron (N.2.15), flupyrsulfuron-methyl-sodium (N.2.16), foramsulfuron (N.2.17), halosulfuron (N.2.18), halosulfuron-methyl (N.2.19), imazosulfuron (N.2.20), iodosulfuron (N.2.21), iodosulfuron-methyl-sodium (N.2.22), iofensulfuron (N.2.23), iofensulfuron-sodium (N.2.24), mesosulfuron (N.2.25), metazosulfuron (N.2.26), metsulfuron (N.2.27), metsulfuron-methyl (N.2.28), nicosulfuron (N.2.29), orthosulfamuron (N.2.30), oxasulfuron (N.2.31), primisulfuron (N.2.32), primisulfuron-methyl (N.2.33), propyrisulfuron (N.2.34), prosulfuron (N.2.35), pyrazosulfuron (N.2.36), pyrazosulfuron-ethyl (N.2.37), rimsulfuron (N.2.38), sulfometuron (N.2.39), sulfometuron-methyl (N.2.40), sulfosulfuron (N.2.41), thifensulfuron (N.2.42), thifensulfuron-methyl (N.2.43), triasulfuron (N.2.44), tribenuron (N.2.45), tribenuronmethyl (N.2.46), trifloxysulfuron (N.2.47), triflusulfuron (N.2.48), triflusulfuron-methyl (N.2.49), tritosulfuron (N.2.50), imazamethabenz (N.2.51), imazamethabenz-methyl (N.2.52), imazamox (N.2.53), imazapic (N.2.54), imazapyr (N.2.55), imazaquin (N.2.56), imazethapyr (N.2.57); cloransulam (N.2.58), cloransulam-methyl (N.2.59), diclosulam (N.2.60), flumetsulam (N.2.61), florasulam (N.2.62), metosulam (N.2.63), penoxsulam (N.2.64), pyrimisulfan (N.2.65) and pyroxsulam (N.2.66); bispyribac (N.2.67), bispyribac-sodium (N.2.68), pyribenzoxim (N.2.69), pyriftalid (N.2.70), pyriminobac (N.2.71), pyriminobac-methyl (N.2.72), pyrithiobac (N.2.73), pyrithiobac-sodium (N.2.74), 4-[[[2-[(4,6-dimethoxy-2-pyrimidinyl)oxy]phenyl]methyl]amino]-benzoic acid-1-methyl¬ethyl ester ((N.2.75) CAS 420138-41-6), 4-[[[2-[(4,6-dimethoxy-2-pyrimidinyl)oxy]phenyl]¬methyl]amino]-benzoic acid propyl ester ((N.2.76) CAS 420138-40-5), N-(4-bromophenyl)-2-[(4,6-dimethoxy-2-pyrimidinyl)oxy]benzenemethanamine ((N.2.77) CAS 420138-01-8); flucarbazone (N.2.78), flucarbazone-sodium (N.2.79), propoxycarbazone (N.2.80), propoxycarbazone-sodium (N.2.81), thiencarbazone (N.2.82), thiencarbazone-methyl (N.2.83), triafamone (N.2.84);

N.3 Photosynthesis inhibitors: amicarbazone (N.3.1); chlorotriazine (N.3.2); ametryn (N.3.3), atrazine (N.3.4), chloridazone (N.3.5), cyanazine (N.3.6), desmetryn (N.3.7), dimethametryn (N.3.8), hexazinone (N.3.9), metribuzin (N.3.10), prometon (N.3.11), prometryn (N.3.12), pro-pazine (N.3.13), simazine (N.3.14), simetryn (N.3.15), terbumeton (N.3.16), terbuthylazin (N.3.17), terbutryn (N.3.18), trietazin (N.3.19); chlorobromuron (N.3.20), chlorotoluron (N.3.21), chloroxuron (N.3.22), dimefuron (N.3.23), diuron (N.3.24), fluometuron (N.3.25), isoproturon (N.3.26), isouron (N.3.27), linuron (N.3.28), metamitron (N.3.29), methabenzthiazuron (N.3.30), metobenzuron (N.3.31), metoxuron (N.3.32), monolinuron (N.3.33), neburon (N.3.34), siduron (N.3.35), tebuthiuron (N.3.36), thiadiazuron (N.3.37), desmedipham (N.3.38), karbutilat (N.3.39), phenmedipham (N.3.40), phenmedipham-ethyl (N.3.41), bromofenoxim (N.3.42), bromoxynil (N.3.43) and its salts and esters, ioxynil (N.3.44) and its salts and esters, bromacil (N.3.45), lenacil (N.3.46), terbacil (N.3.47), bentazon (N.3.48), bentazon-sodium (N.3.49), pyridate (N.3.50), pyridafol (N.3.51), pentanochlor (N.3.52), propanil (N.3.53); diquat (N.3.54), diquat-dibromide (N.3.55), paraquat (N.3.56), paraquat-dichloride (N.3.57), paraquat-dimetilsulfate (N.3.58);

N.4 protoporphyrinogen-IX oxidase inhibitors: acifluorfen (N.4.1), acifluorfen-sodium (N.4.2), azafenidin (N.4.3), bencarbazone (N.4.4), benzfendizone (N.4.5), bifenox (N.4.6), butafenacil (N.4.7), carfentrazone (N.4.8), carfentrazone-ethyl (N.4.9), chlormethoxyfen (N.4.10), cinidon-ethyl (N.4.11), fluazolate (N.4.12), flufenpyr (N.4.13), flufenpyr-ethyl (N.4.14), flumiclorac (N.4.15), flumiclorac-pentyl (N.4.16), flumioxazin (N.4.17), fluoroglycofen (N.4.18), fluoroglycofen-ethyl (N.4.19), fluthiacet (N.4.20), fluthiacet-methyl (N.4.21), fomesafen (N.4.22), halosafen (N.4.23), lactofen (N.4.24), oxadiargyl (N.4.25), oxadiazon (N.4.26), oxyfluorfen (N.4.27), pentoxazone (N.4.28), profluazol (N.4.29), pyraclonil (N.4.30), pyraflufen (N.4.31), pyraflufen-ethyl (N.4.32), saflufenacil (N.4.33), sulfentrazone (N.4.34), thidiazimin (N.4.35), tiafenacil (N.4.36), trifludimoxazin (N.4.37), ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate ((N.4.38) CAS 353292-31-6), N-ethyl-3-(2,6-dichloro-4-trifluoro-methylphenoxy)-5-methyl-1H-pyrazole-1-carboxamide ((N.4.39) CAS 452098-92-9), N tetrahydrofurfuryl-3-(2,6-dichloro-4-trifluoromethylphenoxy)-5-methyl-1H-pyrazole-1-carboxamide ((N.4.40) CAS 915396-43-9), N-ethyl-3-(2-chloro-6-fluoro-4-trifluoromethyl¬phenoxy)-5-methyl-1H-pyrazole-1-carboxamide ((N.4.41) CAS 452099-05-7), N tetrahydro¬furfuryl-3-(2-chloro-6-fluoro-4-trifluoro¬methylphenoxy)-5-methyl-1H-pyrazole-1-carboxamide ((N.4.42) CAS 452100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione ((N.4.43) CAS 451484-50-7), 2-(2,2,7-trifluoro-3-oxo-4-prop-2-ynyl-3,4-dihydro-2H-benzo[1,4]oxazin-6-yl)-4,5,6,7-tetrahydro-isoindole-1,3-dione ((N.4.44) CAS 1300118-96-0), 1-methyl-6-trifluoro¬methyl-3-(2,2,7-tri-fluoro-3-oxo-4-prop-2-ynyl-3,4-dihydro-2H-benzo[1,4]oxazin-6-yl)-1H-pyrimidine-2,4-dione ((N.4.45) CAS 1304113-05-0), methyl (E)-4-[2-chloro-5-[4-chloro-5-(difluoromethoxy)-1H-methyl-pyrazol-3-yl]-4-fluoro-phenoxy]-3-methoxy-but-2-enoate ((N.4.46) CAS 948893-00-3), 3-[7-chloro-5-fluoro-2-(trifluoromethyl)-1H-benzimidazol-4-yl]-1-methyl-6-(trifluoromethyl)-1H-pyrimidine-2,4-dione ((N.4.47) CAS 212754-02-4);

N.5 Bleacher herbicides: beflubutamid (N.5.1), diflufenican (N.5.2), fluridone (N.5.3), flurochloridone (N.5.4), flurtamone (N.5.5), norflurazon (N.5.6), picolinafen (N.5.7), 4-(3-trifluoromethyl¬phenoxy)-2-(4-trifluoromethylphenyl)¬pyrimidine ((N.5.8) CAS 180608-33-7); benzobicyclon (N.5.9), benzofenap (N.5.10), bicyclopyrone (N.5.11), clomazone (N.5.12), fenquintrione (N.5.13), isoxaflutole (N.5.14), mesotrione (N.5.15), pyrasulfotole (N.5.16), pyrazolynate (N.5.17), pyrazoxyfen (N.5.18), sulcotrione (N.5.19), tefuryltrione (N.5.20), tembotrione (N.5.21), tolpyralate (N.5.22), topramezone (N.5.23); aclonifen (N.5.24), amitrole (N.5.25), flumeturon (N.5.26);

N.6 EPSP synthase inhibitors: glyphosate (N.6.1), glyphosate-isopropylammonium (N.6.2), glyposate-potassium (N.6.3), glyphosate-trimesium (sulfosate) (N.6.4);

N.7 Glutamine synthase inhibitors: bilanaphos (bialaphos) (N.7.1), bilanaphos-sodium (N.7.2), glufosinate (N.7.3), glufosinate-P (N.7.4), glufosinate-ammonium (N.7.5);

N.8 DHP synthase inhibitors: asulam (N.8.1);

N.9 Mitosis inhibitors: benfluralin (N.9.1), butralin (N.9.2), dinitramine (N.9.3), ethalfluralin (N.9.4), fluchloralin (N.9.5), oryzalin (N.9.6), pendimethalin (N.9.7), prodiamine (N.9.8), trifluralin (N.9.9); amiprophos (N.9.10), amiprophos-methyl (N.9.11), butamiphos (N.9.12); chlorthal (N.9.13), chlorthal-dimethyl (N.9.14), dithiopyr (N.9.15), thiazopyr (N.9.16), propyzamide (N.9.17), tebutam (N.9.18); carbetamide (N.9.19), chlorpropham (N.9.20), flamprop (N.9.21), flamprop-isopropyl (N.9.22), flamprop-methyl (N.9.23), flamprop-M-isopropyl (N.9.24), flamprop-M-methyl (N.9.25), propham (N.9.26);

N.10 VLCFA inhibitors: acetochlor (N.10.1), alachlor (N.10.2), butachlor (N.10.3), dimethachlor (N.10.4), dimethenamid (N.10.5), dimethenamid-P (N.10.6), metazachlor (N.10.7), metolachlor (N.10.8), metolachlor-S(N.10.9), pethoxamid (N.10.10), pretilachlor (N.10.11), propachlor (N.10.12), propisochlor (N.10.13), thenylchlor (N.10.14), flufenacet (N.10.15), mefenacet (N.10.16), diphenamid (N.10.17), naproanilide (N.10.18), napropamide (N.10.19), napropamide-M (N.10.20), fentrazamide (N.10.21), anilofos (N.10.22), cafenstrole (N.10.23), fenoxasulfone (N.10.24), ipfencarbazone (N.10.25), piperophos (N.10.26), pyroxasulfone (N.10.27), isoxazoline compounds of the formulae II.1, II.2, II.3, II.4, II.5, II.6, II.7, II.8 and II.9

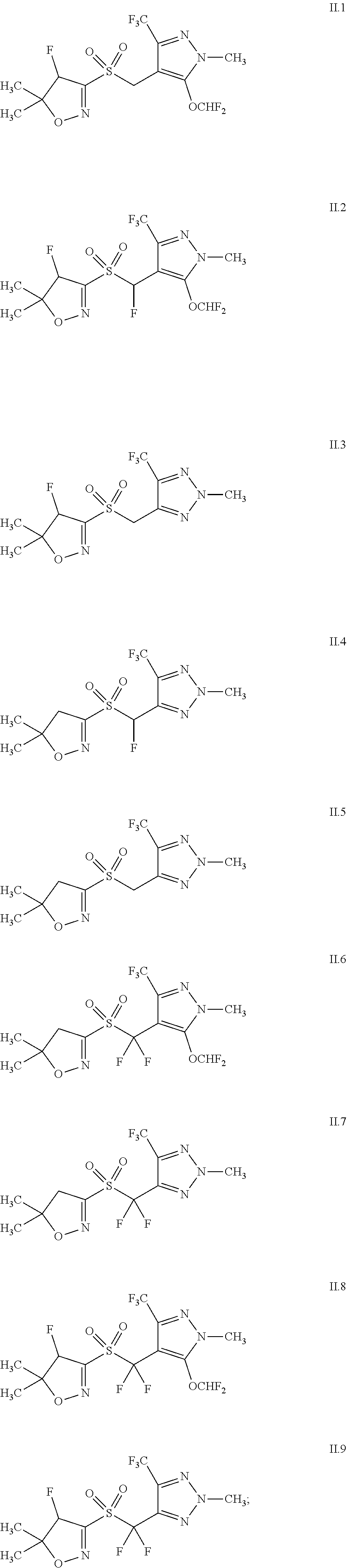

N.11 Cellulose biosynthesis inhibitors: chlorthiamid (N.11.1), dichlobenil (N.11.2), flupoxam (N.11.3), indaziflam (N.11.4), isoxaben (N.11.5), triaziflam (N.11.6), 1-cyclohexyl-5-pentafluorphenyloxy-14-[1,2,4,6]thiatriazin-3-ylamine ((N.11.7) CAS 175899-01-1);

N.12 Decoupler herbicides: dinoseb (N.12.1), dinoterb (N.12.2), DNOC (N.12.3) and its salts;

N.13 Auxinic herbicides: 2,4-D (N.13.1) and its salts and esters, clacyfos (N.13.2), 2,4-DB (N.13.3) and its salts and esters, aminocyclopyrachlor (N.13.4) and its salts and esters, aminopyralid (N.13.5) and its salts such as aminopyralid-dimethylammonium (N.13.6), aminopyralid-tris(2-hydroxypropyl)ammonium (N.13.7) and its esters, benazolin (N.13.8), benazolin-ethyl (N.13.9), chloramben (N.13.10) and its salts and esters, clomeprop (N.13.11), clopyralid (N.13.12) and its salts and esters, dicamba (N.13.13) and its salts and esters, dichlorprop (N.13.14) and its salts and esters, dichlorprop-P (N.13.15) and its salts and esters, fluroxypyr (N.13.16), fluroxypyr-butometyl (N.13.17), fluroxypyr-meptyl (N.13.18), halauxifen (N.13.) and its salts and esters (CAS 943832-60-8); MCPA (N.13.) and its salts and esters, MCPA-thioethyl (N.13.19), MCPB (N.13.20) and its salts and esters, mecoprop (N.13.21) and its salts and esters, mecoprop-P (N.13.22) and its salts and esters, picloram (N.13.23) and its salts and esters, quinclorac (N.13.24), quinmerac (N.13.25), TBA (2,3,6) (N.13.26) and its salts and esters, triclopyr (N.13.27) and its salts and esters, 4-amino-3-chloro-6-(4-chloro-2-fluoro-3-methoxyphenyl)-5-fluoropyridine-2-carboxylic acid (N.13.28), benzyl 4-amino-3-chloro-6-(4-chloro-2-fluoro-3-methoxyphenyl)-5-fluoropyridine-2-carboxylate ((N.13.29) CAS 1390661-72-9);

N.14 Auxin transport inhibitors: diflufenzopyr (N.14.1), diflufenzopyr-sodium (N.14.2), naptalam (N.14.3) and naptalam-sodium (N.14.4);

N.15 Other herbicides: bromobutide (N.15.1), chlorflurenol (N.15.2), chlorflurenol-methyl (N.15.3), cinmethylin (N.15.4), cumyluron (N.15.5), cyclopyrimorate ((N.15.6) CAS 499223-49-3) and its salts and esters, dalapon (N.15.7), dazomet (N.15.8), difenzoquat (N.15.9), difenzoquat-metilsulfate (N.15.10), dimethipin (N.15.11), DSMA (N.15.12), dymron (N.15.13), endothal (N.15.14) and its salts, etobenzanid (N.15.15), flurenol (N.15.16), flurenol-butyl (N.15.17), flurprimidol (N.15.18), fosamine (N.15.19), fosamine-ammonium (N.15.20), indanofan (N.15.21), maleic hydrazide (N.15.22), mefluidide (N.15.23), metam (N.15.24), methiozolin ((N.15.25) CAS 403640-27-7), methyl azide (N.15.26), methyl bromide (N.15.27), methyl-dymron (N.15.28), methyl iodide (N.15.29), MSMA (N.15.30), oleic acid (N.15.31), oxaziclomefone (N.15.32), pelargonic acid (N.15.33), pyributicarb (N.15.34), quinoclamine (N.15.35), tridiphane (N.15.36);

O) Insecticides from Classes 0.1 to 0.29

O.1 Acetylcholine esterase (AChE) inhibitors: aldicarb (O.1.1), alanycarb (O.1.2), bendiocarb (O.1.3), benfuracarb (O.1.4), butocarboxim (O.1.5), butoxycarboxim (O.1.6), carbaryl (O.1.7), carbofuran (O.1.8), carbosulfan (O.1.9), ethiofencarb (O.1.10), fenobucarb (O.1.11), formetanate (O.1.12), furathiocarb (O.1.13), isoprocarb (O.1.14), methiocarb (O.1.15), methomyl (O.1.16), metolcarb (O.1.17), oxamyl (O.1.18), pirimicarb (O.1.19), propoxur (O.1.20), thiodicarb (O.1.21), thiofanox (O.1.22), trimethacarb (O.1.23), XMC (O.1.24), xylylcarb (O.1.25) and triazamate (O.1.26); acephate (O.1.27), azamethiphos (O.1.28), azinphos-ethyl (O.1.29), azinphosmethyl (O.1.30), cadusafos (O.1.31), chlorethoxyfos (O.1.32), chlorfenvinphos (O.1.33), chlormephos (O.1.34), chlorpyrifos (O.1.35), chlorpyrifosmethyl (O.1.36), coumaphos (O.1.37), cyanophos (O.1.38), demeton-S-methyl (O.1.39), diazinon (O.1.40), dichlorvos/DDVP (O.1.41), dicrotophos (O.1.42), dimethoate (O.1.43), dimethylvinphos (O.1.44), disulfoton (O.1.45), EPN (O.1.46), ethion (O.1.47), ethoprophos (O.1.48), famphur (O.1.49), fenamiphos (O.1.50), fenitrothion (O.1.51), fenthion (O.1.52), fosthiazate (O.1.53), heptenophos (O.1.54), imicyafos (O.1.55), isofenphos (O.1.56), isopropyl 0-(methoxyaminothio-phosphoryl) salicylate (O.1.57), isoxathion (O.1.58), malathion (O.1.59), mecarbam (O.1.60), methamidophos (O.1.61), methidathion (O.1.62), mevinphos (O.1.63), monocrotophos (O.1.64), naled (O.1.65), omethoate (O.1.66), oxydemeton-methyl (O.1.67), parathion (O.1.68), parathion-methyl (O.1.69), phenthoate (O.1.70), phorate (O.1.71), phosalone (O.1.72), phosmet (O.1.73), phosphamidon (O.1.74), phoxim (O.1.75), pirimiphos-methyl (O.1.76), profenofos (O.1.77), propetamphos (O.1.78), prothiofos (O.1.79), pyraclofos (O.1.80), pyridaphenthion (O.1.81), quinalphos (O.1.82), sulfotep (O.1.83), tebupirimfos (O.1.84), temephos (O.1.85), terbufos (O.1.86), tetrachlorvinphos (O.1.87), thiometon (O.1.88), triazophos (O.1.89), trichlorfon (O.1.90), vamidothion (O.1.91);

O.2 GABA-gated chloride channel antagonists: endosulfan (O.2.1), chlordane (O.2.2); ethiprole (O.2.3), fipronil (O.2.4), flufiprole (O.2.5), pyrafluprole (O.2.6), pyriprole (O.2.7);

O.3 Sodium channel modulators: acrinathrin (O.3.1), allethrin (O.3.2), d-cis-trans allethrin (O.3.3), d-trans allethrin (O.3.4), bifenthrin (O.3.5), bioallethrin (O.3.6), bioallethrin S-cylclopentenyl (O.3.7), bioresmethrin (O.3.8), cycloprothrin (O.3.9), cyfluthrin (O.3.10), beta-cyfluthrin (O.3.11), cyhalothrin (O.3.12), lambda-cyhalothrin (O.3.13), gamma-cyhalothrin (O.3.14), cypermethrin (O.3.15), alpha-cypermethrin (O.3.16), beta-cypermethrin (O.3.17), theta-cypermethrin (O.3.18), zeta-cypermethrin (O.3.19), cyphenothrin (O.3.20), deltamethrin (O.3.21), empenthrin (O.3.22), esfenvalerate (O.3.23), etofenprox (O.3.24), fenpropathrin (O.3.25), fenvalerate (O.3.26), flucythrinate (O.3.27), flumethrin (O.3.28), tau-fluvalinate (O.3.29), halfenprox (O.3.30), heptafluthrin (O.3.31), imiprothrin (O.3.32), meperfluthrin (O.3.33), metofluthrin (O.3.34), momfluorothrin (O.3.35), permethrin (O.3.36), phenothrin (O.3.37), prallethrin (O.3.38), profluthrin (O.3.39), pyrethrin (pyrethrum) (O.3.40), resmethrin (O.3.41), silafluofen (O.3.42), tefluthrin (O.3.43), tetramethylfluthrin (O.3.44), tetramethrin (O.3.45), tralomethrin (O.3.46) and transfluthrin (O.3.47); DDT (O.3.48), methoxychlor (O.3.49);

O.4 Nicotinic acetylcholine receptor agonists (nAChR): acetamiprid (O.4.1), clothianidin (O.4.2), cycloxaprid (O.4.3), dinotefuran (O.4.4), imidacloprid (O.4.5), nitenpyram (O.4.6), thiacloprid (O.4.7), thiamethoxam (O.4.8); (2E)-1-[(6-chloropyridin-3-yl)methyl]-N′-nitro-2-pentylidenehydrazinecarboximidamide (O.4.9); 1-[(6-chloropyridin-3-yl)methyl]-7-methyl-8-nitro-5-propoxy-1,2,3,5,6,7-hexahydroimidazo[1,2-a]pyridine (O.4.10); nicotine (O.4.11);

O.5 Nicotinic acetylcholine receptor allosteric activators: spinosad (O.5.1), spinetoram (O.5.2);

O.6 Chloride channel activators: abamectin (O.6.1), emamectin benzoate (O.6.2), ivermectin (O.6.3), lepimectin (O.6.4), milbemectin (O.6.5);

O.7 Juvenile hormone mimics: hydroprene (O.7.1), kinoprene (O.7.2), methoprene (O.7.3); fenoxycarb (O.7.4), pyriproxyfen (O.7.5);

O.8 miscellaneous non-specific (multi-site) inhibitors: methyl bromide (O.8.1) and other alkyl halides; chloropicrin (O.8.2), sulfuryl fluoride (O.8.3), borax (O.8.4), tartar emetic (O.8.5);

O.9 Selective homopteran feeding blockers: pymetrozine (O.9.1), flonicamid (O.9.2);

O.10 Mite growth inhibitors: clofentezine (O.10.1), hexythiazox (O.10.2), diflovidazin (O.10.3); etoxazole (O.10.4);

O.11 Microbial disruptors of insect midgut membranes: the Bt crop proteins: Cry1Ab, Cry1Ac, Cry1Fa, Cry2Ab, mCry3A, Cry3Ab, Cry3Bb, Cry34/35Ab1;

O.12 Inhibitors of mitochondrial ATP synthase: diafenthiuron (O.12.1); azocyclotin (O.12.2), cyhexatin (O.12.3), fenbutatin oxide (O.12.4), propargite (O.12.5), tetradifon (O.12.6);

O.13 Uncouplers of oxidative phosphorylation via disruption of the proton gradient: chlorfenapyr (O.13.1), DNOC (O.13.2), sulfluramid (O.13.3);

O.14 Nicotinic acetylcholine receptor (nAChR) channel blockers: bensultap (O.14.1), cartap hydrochloride (O.14.2), thiocyclam (O.14.3), thiosultap sodium (O.14.4);

O.15 Inhibitors of the chitin biosynthesis type 0: bistrifluron (O.15.1), chlorfluazuron (O.15.2), diflubenzuron (O.15.3), flucycloxuron (O.15.4), flufenoxuron (O.15.5), hexaflumuron (O.15.6), lufenuron (O.15.7), novaluron (O.15.8), noviflumuron (O.15.9), teflubenzuron (O.15.10), triflumuron (O.15.11);

O.16 Inhibitors of the chitin biosynthesis type 1: buprofezin (O.16.1);

O.17 Moulting disruptors: cyromazine (O.17.1);

O.18 Ecdyson receptor agonists: methoxyfenozide (O.18.1), tebufenozide (O.18.2), halofenozide (O.18.3), fufenozide (O.18.4), chromafenozide (O.18.5);

O.19 Octopamin receptor agonists: amitraz (O.19.1);

O.20 Mitochondrial complex III electron transport inhibitors: hydramethylnon (O.20.1), acequinocyl (O.20.2), fluacrypyrim (O.20.3);

O.21 Mitochondrial complex I electron transport inhibitors: fenazaquin (O.21.1), fenpyroximate (O.21.2), pyrimidifen (O.21.3), pyridaben (O.21.4), tebufenpyrad (O.21.5), tolfenpyrad (O.21.6); rotenone (O.21.7);

O.22 Voltage-dependent sodium channel blockers: indoxacarb (O.22.1), metaflumizone (O.22.2),2-[2-(4-cyanophenyl)-1-[3-(trifluoromethyl)phenyl]ethylidene]-N-[4-(difluoromethoxy)phenyl]-hydrazinecarboxamide(O.22.3), N-(3-chloro-2-methylphenyl)-2-[(4-chlorophenyl)-[4-[methyl(methylsulfonyl)amino]phenyl]methylene]-hydrazinecarboxamide (O.22.4);

O.23 Inhibitors of the of acetyl CoA carboxylase: spirodiclofen (O.23.1), spiromesifen (O.23.2), spirotetramat (O.23.3);

O.24 Mitochondrial complex IV electron transport inhibitors: aluminium phosphide (O.24.1), calcium phosphide (O.24.2), phosphine (O.24.3), zinc phosphide (O.24.4), cyanide (O.24.5);

O.25 Mitochondrial complex II electron transport inhibitors: cyenopyrafen (O.25.1), cyflumetofen (O.25.2);

O.26 Ryanodine receptor-modulators: flubendiamide (O.26.1), chlorantraniliprole (O.26.2), cyantraniliprole (O.26.3), cyclaniliprole (O.26.4), tetraniliprole (O.26.5); (R)-3-chloro-N1-{2-methyl-4-[1,2,2,2-tetrafluoro-1-(trifluoromethyl)ethyl]phenyl}-N2-(1-methyl-2-methylsulfonylethyl)phthalamide (O.26.6), (S)-3-chloro-N1-{2-methyl-4-[1,2,2,2-tetrafluoro-1-(trifluoromethyl)ethyl]phenyl}-N2-(1-methyl-2-methylsulfonylethyl)phthalamide (O.26.7), methyl-2-[3,5-dibromo-2-({[3-bromo-1-(3-chloropyridin-2-yl)-1H-pyrazol-5-yl]carbonyl}-amino)benzoyl]-1,2-dimethylhydrazinecarboxylate (O.26.8); N-[4,6-dichloro-2-[(diethyl-lambda-4-sulfanylidene)carbamoyl]-phenyl]-2-(3-chloro-2-pyridyl)-5-(trifluoromethyl)pyrazole-3-carboxamide (O.26.9); N-[4-chloro-2-[(diethyl-ambda-4-sulfanylidene)carbamoyl]-6-methyl-phenyl]-2-(3-chloro-2-pyridyl)-5-(trifluoromethyl)pyrazole-3-carboxamide (O.26.10); N-[4-chloro-2-[(di-2-propyl-lambda-4-sulfanylidene)carbamoyl]-6-methyl-phenyl]-2-(3-chloro-2-pyridyl)-5-(trifluoromethyl)pyrazole-3-carboxamide (O.26.11); N-[4,6-dichloro-2-[(di-2-propyl-lambda-4-sulfanylidene)carbamoyl]-phenyl]-2-(3-chloro-2-pyridyl)-5-(trifluoromethyl)pyrazole-3-carboxamide (O.26.12); N-[4,6-dibromo-2-[(diethyl-ambda-4-sulfanylidene)carbamoyl]-phenyl]-2-(3-chloro-2-pyridyl)-5-(trifluoromethyl)pyrazoe-3-carboxamide (O.26.13); N-[2-(5-amino-1,3,4-thiadiazol-2-yl)-4-chloro-6-methylphenyl]-3-bromo-1-(3-chloro-2-pyridinyl)-1H-pyrazole-5-carboxamide (O.26.14); 3-chloro-1-(3-chloro-2-pyridinyl)-N-[2,4-dichloro-6-[[(1-cyano-1-methylethyl)amino]carbonyl]phenyl]-1H-pyrazole-5-carboxamide (O.26.15); 3-bromo-N-[2,4-dichloro-6-(methylcarbamoyl)phenyl]-1-(3,5-dichloro-2-pyridyl)-1H-pyrazole-5-carboxamide (O.26.16); N-[4-chloro-2-[[(1,1-dimethylethyl)amino]carbonyl]-6-methylphenyl]-1-(3-chloro-2-pyridinyl)-3-(fluoromethoxy)-1H-pyrazole-5-carboxamide (O.26.17); cyhalodiamide (O.26.18);

O.27. insecticidal active compounds of unknown or uncertain mode of action: afidopyropen (O.27.1), afoxolaner (O.27.2), azadirachtin (O.27.3), amidoflumet (O.27.4), benzoximate (O.27.5), bifenazate (O.27.6), broflanilide (O.27.7), bromopropylate (O.27.8), chinomethionat (O.27.9), cryolite (O.27.10), dicloromezotiaz (O.27.11), dicofol (O.27.12), flufenerim (O.27.13), flometoquin (O.27.14), fluensulfone (O.27.15), fluhexafon (O.27.16), fluopyram (O.27.17), flupyradifurone (O.27.18), fluralaner (O.27.19), metoxadiazone (O.27.20), piperonyl butoxide (O.27.21), pyflubumide (O.27.22), pyridalyl (O.27.23), pyrifluquinazon (O.27.24), sulfoxaflor (O.27.25), tioxazafen (O.27.26), triflumezopyrim (O.27.27), 11-(4-chloro-2,6-dimethylphenyl)-12-hydroxy-1,4-dioxa-9-azadispiro[4.2.4.2]-tetradec-11-en-10-one (O.27.28), 3-(4′-fluoro-2,4-dimethylbiphenyl-3-yl)-4-hydroxy-8-oxa-1-azaspiro[4.5]dec-3-en-2-one (O.27.28), 1-[2-fluoro-4-methyl-5-[(2,2,2-trifluoroethyl)sulfinyl]phenyl]-3-(trifluoromethyl)-1H-1,2,4-triazole-5-amine (O.27.29), (E/Z)—N-[1-[(6-chloro-3-pyridyl)methyl]-2-pyridylidene]-2,2,2-trifluoro-acetamide (O.27.31); (E/Z)—N-[1-[(6-chloro-5-fluoro-3-pyridyl)methyl]-2-pyridylidene]-2,2,2-trifluoro-acetamide (O.27.32); (E/Z)-2,2,2-trifluoro-N-[1-[(6-fluoro-3-pyridyl)methyl]-2-pyridylidene]acetamide (O.27.33); (E/Z)—N-[1-[(6-bromo-3-pyridyl)methyl]-2-pyridylidene]-2,2,2-trifluoro-acetamide (O.27.34); (E/Z)—N-[1-[1-(6-chloro-3-pyridyl)ethyl]-2-pyridylidene]-2,2,2-trifluoro-acetamide (O.27.35); (E/Z)—N-[1-[(6-chloro-3-pyridyl)methyl]-2-pyridylidene]-2,2-difluoro-acetamide (O.27.36); (E/Z)-2-chloro-N-[1-[(6-chloro-3-pyridyl)methyl]-2-pyridylidene]-2,2-difluoro-acetamide (O.27.37); (E/Z)—N-[1-[(2-chloropyrimidin-5-yl)methyl]-2-pyridylidene]-2,2,2-trifluoro-acetamide (O.27.38); (E/Z)—N-[1-[(6-chloro-3-pyridyl)methyl]-2-pyridylidene]-2,2,3,3,3-pentafluoro-propanamide (O.27.39); N-[1-[(6-chloro-3-pyridyl)methyl]-2-pyridylidene]-2,2,2-trifluoro-thioacetamide (O.27.40); N-[1-[(6-chloro-3-pyridyl)methyl]-2-pyridylidene]-2,2,2-trifluoro-N′-isopropyl-acetamidine (O.27.41); fluazaindolizine (O.27.42); 4-[5-(3,5-dichlorophenyl)-5-(trifluoromethyl)-4H-isoxazol-3-yl]-2-methyl-N-(1-oxothietan-3-yl)benzamide (O.27.43); fluxametamide (O.27.44); 5-[3-[2,6-dichloro-4-(3,3-dichloroallyloxy)phenoxy]propoxy]-1H-pyrazole (O.27.45); 3-(benzoylmethylamino)-N-[2-bromo-4-[1,2,2,3,3,3-hexafluoro-1-(trifluoromethyl)propyl]-6-(trifluoromethyl)phenyl]-2-fluoro-benzamide (O.27.46); 3-(benzoylmethylamino)-2-fluoro-N-[2-iodo-4-[1,2,2,2-tetrafluoro-1-(trifluoromethyl)ethyl]-6-(trifluoromethyl)phenyl]-benzamide (O.27.47); N-[3-[[[2-iodo-4-[1,2,2,2-tetrafluoro-1-(trifluoromethyl)ethyl]-6-(trifluoromethyl)phenyl]amino]carbonyl]phenyl]-N-methyl-benzamide (O.27.48); N-[3-[[[2-bromo-4-[1,2,2,2-tetrafluoro-1-(trifluoromethyl)ethyl]-6-(trifluoromethyl)phenyl]amino]carbonyl]-2-fluorophenyl]-4-fluoro-N-methyl-benzamide (O.27.49); 4-fluoro-N-[2-fluoro-3-[[[2-iodo-4-[1,2,2,2-tetrafluoro-1-(trifluoromethyl)ethyl]-6-(trifluoromethyl)phenyl]amino]carbonyl]phenyl]-N-methyl-benzamide (O.27.50); 3-fluoro-N-[2-fluoro-3-[[[2-iodo-4-[1,2,2,2-tetrafluoro-1-(trifluoromethyl)ethyl]-6-(trifluoromethyl)phenyl]amino]carbonyl]phenyl]-N-methyl-benzamide (O.27.51); 2-chloro-N-[3-[[[2-iodo-4-[1,2,2,2-tetrafluoro-1-(trifluoromethyl)ethyl]-6-(trifluoromethyl)phenyl]amino]carbonyl]phenyl]-3-pyridinecarboxamide (O.27.52); 4-cyano-N-[2-cyano-5-[[2,6-dibromo-4-[1,2,2,3,3,3-hexafluoro-1-(trifluoromethyl)propyl]phenyl]carbamoyl]phenyl]-2-methyl-benzamide (O.27.53); 4-cyano-3-[(4-cyano-2-methyl-benzoyl)amino]-N-[2,6-dichloro-4-[1,2,2,3,3,3-hexafluoro-1-(trifluoromethyl)propyl]phenyl]-2-fluoro-benzamide (O.27.54); N-[5-[[2-chloro-6-cyano-4-[1,2,2,3,3,3-hexafluoro-1-(trifluoromethyl)propyl]-phenyl]carbamoyl]-2-cyano-phenyl]-4-cyano-2-methyl-benzamide (O.27.55); N-[5-[[2-bromo-6-chloro-4-[2,2,2-trifluoro-1-hydroxy-1-(trifluoromethyl)ethyl]phenyl]carbamoyl]-2-cyano-phenyl]-4-cyano-2-methyl-benzamide (O.27.56); N-[5-[[2-bromo-6-chloro-4-[1,2,2,3,3,3-hexafluoro-1-(trifluoromethyl)propyl]phenyl]carbamoyl]-2-cyano-phenyl]-4-cyano-2-methyl-benzamide (O.27.57); 4-cyano-N-[2-cyano-5-[[2,6-dichloro-4-[1,2,2,3,3,3-hexafluoro-1-(trifluoromethyl)-propyl]phenyl]carbamoyl]phenyl]-2-methyl-benzamide (O.27.58); 4-cyano-N-[2-cyano-5-[[2,6-dichloro-4-[1,2,2,2-tetrafluoro-1-(trifluoromethyl)ethyl]phenyl]carbamoyl]phenyl]-2-methyl-benzamide (O.27.59); N-[5-[[2-bromo-6-chloro-4-[1,2,2,2-tetrafluoro-1-(trifluoromethyl)ethyl]phenyl]carbamoyl]-2-cyano-phenyl]-4-cyano-2-methyl-benzamide (O.27.60); 2-(1,3-dioxan-2-yl)-6-[2-(3-pyridinyl)-5-thiazolyl]-pyridine; 2-[6-[2-(5-fluoro-3-pyridinyl)-5-thiazolyl]-2-pyridinyl]-pyrimidine (O.27.61); 2-[6-[2-(3-pyridinyl)-5-thiazolyl]-2-pyridinyl]-pyrimidine (O.27.62); N-methylsulfonyl-6-[2-(3-pyridyl)thiazol-5-yl]pyridine-2-carboxamide (O.27.63); N-methylsulfonyl-6-[2-(3-pyridyl)thiazol-5-yl]pyridine-2-carboxamide (O.27.64); N-ethyl-N-[4-methyl-2-(3-pyridyl)thiazol-5-yl]-3-methylthio-propanamide (O.27.65); N-methyl-N-[4-methyl-2-(3-pyridyl)thiazol-5-yl]-3-methylthio-propanamide (O.27.66); N,2-dimethyl-N-[4-methyl-2-(3-pyridyl)thiazol-5-yl]-3-methylthio-propanamide (O.27.67); N-ethyl-2-methyl-N-[4-methyl-2-(3-pyridyl)thiazol-5-yl]-3-methylthio-propanamide (O.27.68); N-[4-chloro-2-(3-pyridyl)thiazol-5-yl]-N-ethyl-2-methyl-3-methylthio-propanamide (O.2769.); N-[4-chloro-2-(3-pyridyl)thiazol-5-yl]-N,2-dimethyl-3-methylthio-propanamide (O.27.70); N-[4-chloro-2-(3-pyridyl)thiazol-5-yl]-N-methyl-3-methylthio-propanamide (O.27.71); N-[4-chloro-2-(3-pyridyl)thiazol-5-yl]-N-ethyl-3-methylthio-propanamide (O.27.72); 1-[(6-chloro-3-pyridinyl)methyl]-1,2,3,5,6,7-hexahydro-5-methoxy-7-methyl-8-nitro-imidazo[1,2-a]pyridine (O.27.73); 1-[(6-chloropyridin-3-yl)methyl]-7-methyl-8-nitro-1,2,3,5,6,7-hexahydroimidazo[1,2-a]pyridin-5-ol (O.27.74); 1-isopropyl-N,5-dimethyl-N-pyridazin-4-yl-pyrazole-4-carboxamide (O.27.75); 1-(1,2-dimethylpropyl)-N-ethyl-5-methyl-N-pyridazin-4-yl-pyrazole-4-carboxamide (O.27.76); N,5-dimethyl-N-pyridazin-4-yl-1-(2,2,2-trifluoro-1-methyl-ethyl)pyrazole-4-carboxamide (O.27.77); 1-[1-(1-cyanocyclopropyl)ethyl]-N-ethyl-5-methyl-N-pyridazin-4-yl-pyrazole-4-carboxamide (O.27.78); N-ethyl-1-(2-fluoro-1-methyl-propyl)-5-methyl-N-pyridazin-4-yl-pyrazole-4-carboxamide (O.27.79); 1-(1,2-dimethylpropyl)-N,5-dimethyl-N-pyridazin-4-yl-pyrazole-4-carboxamide (O.27.80); 1-[1-(1-cyanocyclopropyl)ethyl]-N,5-dimethyl-N-pyridazin-4-yl-pyrazole-4-carboxamide (O.27.81); N-methyl-1-(2-fluoro-1-methyl-propyl]-5-methyl-N-pyridazin-4-yl-pyrazole-4-carboxamide (O.27.82); 1-(4,4-difluorocyclohexyl)-N-ethyl-5-methyl-N-pyridazin-4-yl-pyrazole-4-carboxamide (O.27.83); 1-(4,4-difluorocyclohexyl)-N,5-dimethyl-N-pyridazin-4-yl-pyrazole-4-carboxamide (O.27.84), N-(1-methylethyl)-2-(3-pyridinyl)-2H-indazole-4-carboxamide (O.27.85); N-cyclopropyl-2-(3-pyridinyl)-2H-indazole-4-carboxamide (O.27.86); N-cyclohexyl-2-(3-pyridinyl)-2H-indazole-4-carboxamide (O.27.87); 2-(3-pyridinyl)-N-(2,2,2-trifluoroethyl)-2H-indazole-4-carboxamide (O.27.88); 2-(3-pyridinyl)-N-[(tetrahydro-2-furanyl)methyl]-2H-indazole-5-carboxamide (O.27.89); methyl 2-[[2-(3-pyridinyl)-2H-indazol-5-yl]carbonyl]hydrazinecarboxylate (O.27.90); N-[(2,2-difluorocyclopropyl)methyl]-2-(3-pyridinyl)-2H-indazole-5-carboxamide (O.27.91); N-(2,2-difluoropropyl)-2-(3-pyridinyl)-2H-indazole-5-carboxamide (O.27.92); 2-(3-pyridinyl)-N-(2-pyrimidinylmethyl)-2H-indazole-5-carboxamide (O.27.93); N-[(5-methyl-2-pyrazinyl)methyl]-2-(3-pyridinyl)-2H-indazole-5-carboxamide (O.27.94), N-[3-chloro-1-(3-pyridyl)pyrazol-4-yl]-N-ethyl-3-(3,3,3-trifluoropropylsulfanyl)-propanamide (O.27.95); N-[3-chloro-1-(3-pyridyl)pyrazol-4-yl]-N-ethyl-3-(3,3,3-trifluoropropylsulfinyl)propanamide (O.27.96); N-[3-chloro-1-(3-pyridyl)pyrazol-4-yl]-3-[(2,2-di-fluorocyclopropyl)methylsulfanyl]-N-ethyl-propanamide (O.27.97); N-[3-chloro-1-(3-pyridyl)pyrazol-4-yl]-3-[(2,2-difluorocyclopropyl)methylsulfinyl]-N-ethyl-propanamide (O.27.98); sarolaner (O.27.99), lotilaner (O.27.100);

in a weight ratio of from 100:1 to 1:100.

The invention also relates to a method for controlling phytopathogenic harmful fungi using mixtures comprising as component 1) of at least one compound of the formula I and comprising as component 2) at least one compound II in a weight ratio of from 100:1 to 1:100; to the use of mixtures comprising compounds I and compounds II for controlling phytopathogenic harmful fungi; to agrochemical compositions comprising these mixtures; and to agrochemical compositions further comprising seed.

Practical agricultural experience has shown that the repeated and exclusive application of an individual active compound in the control of harmful fungi leads in many cases to a rapid selection of those fungus strains which have developed natural or adapted resistance against the active compound in question. Effective control of these fungi with the active compound in question is then no longer possible.

To reduce the risk of the selection of resistant fungus strains, mixtures of different active compounds are conventionally employed for controlling harmful fungi. By combining active compounds having different mechanisms of action, it is possible to ensure successful control over a relatively long period of time.

It is an object of the present invention to provide, with a view to effective resistance management and effective control of phytopathogenic harmful fungi, at application rates which are as low as possible, compositions which, at a reduced total amount of active compounds applied, have improved activity against the harmful fungi (synergistic mixtures) and a broadened activity spectrum, in particular for certain indications.

We have accordingly found that this object is achieved by the compositions, defined herein, comprising at least one compound I and at least one compound II.

Moreover, we have found that simultaneous, that is joint or separate, application of a compound I and a compound II or successive application of a compound I and of compound II allows better control of harmful fungi than is possible with the individual compounds alone (synergistic mixtures). Compounds I and/or the compounds II can be present in different crystal modifications, which may differ in biological activity.

The term “compounds I” refers to compounds of formula I.

In the definitions of the variables given above, collective terms are used which are generally representative for the substituents in question. The term “Cₙ-Cₘ” indicates the number of carbon atoms possible in each case in the substituent or substituent moiety in question.

The term “halogen” refers to fluorine, chlorine, bromine and iodine.

The term “C₁-C₆-alkyl” refers to a straight-chained or branched saturated hydrocarbon group having 1 to 6 carbon atoms, for example methyl, ethyl, propyl, 1-methylethyl, butyl, 1-methylpropyl, 2-methylpropyl, and 1,1-dimethylethyl.

The term “C₂-C₆-alkenyl” refers to a straight-chain or branched unsaturated hydrocarbon radical having 2 to 6 carbon atoms and a double bond in any position, such as ethenyl, 1-propenyl, 2-propenyl (allyl), 1-methylethenyl, 1-butenyl, 2-butenyl, 3-butenyl, 1-methyl-1-propenyl, 2-methyl-1-propenyl, 1-methyl-2-propenyl, 2-methyl-2-propenyl.

The term “C₂-C₆-alkynyl” refers to a straight-chain or branched unsaturated hydrocarbon radical having 2 to 6 carbon atoms and containing at least one triple bond, such as ethynyl, 1-propynyl, 2-propynyl (propargyl), 1-butynyl, 2-butynyl, 3-butynyl, 1-methyl-2-propynyl.

The term “C₁-C₆-haloalkyl” refers to a straight-chained or branched alkyl group having 1 to 6 carbon atoms (as defined above), wherein some or all of the hydrogen atoms in these groups may be replaced by halogen atoms as mentioned above, for example chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl and pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, CH₂—C₂F₅, CF₂—C₂F₅, CF(CF₃)₂, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl.

The term “C₁-C₆-alkoxy” refers to a straight-chain or branched alkyl group having 1 to 6 carbon atoms (as defined above) which is bonded via an oxygen, at any position in the alkyl group, for example methoxy, ethoxy, n-propoxy, 1-methylethoxy, butoxy, 1-methylpropoxy, 2-methylpropoxy or 1,1-dimethylethoxy.

The term “C₃-C₈-cycloalkyl” refers to monocyclic saturated hydrocarbon radicals having 3 to 8 carbon ring members such as cyclopropyl (C₃H₅), cyclobutyl, cyclopentyl, cyclohexyl, cycloheptyl or cyclooctyl.

The term “aliphatic” refers to compounds or radicals composed of carbon and hydrogen and which are non-aromatic compounds. An “alicyclic” compound or radical is an organic compound that is both aliphatic and cyclic. They contain one or more all-carbon rings which may be either saturated or unsaturated, but do not have aromatic character.

The term “cyclic group” refers to a radical which is an alicyclic ring or an aromatic ring, such as, for example, phenyl or heteroaryl.

The term “and wherein any of the aliphatic or cyclic groups are unsubstituted or substituted with . . . ” refers to aliphatic groups, cyclic groups and groups, which contain an aliphatic and a cyclic moiety in one group, such as in, for example, C₃-C₅-cycloalkyl-C₁-C₄-alkyl; therefore a group which contains an aliphatic and a cyclic moiety both of these moieties may be substituted or unsubstituted independently of each other.

The term “phenyl” refers to an aromatic ring systems including six carbon atoms (commonly referred to as benzene ring).

Agriculturally acceptable salts of the active compounds I, and III encompass especially the salts of those cations or the acid addition salts of those acids whose cations and anions, respectively, have no adverse effect on the fungicidal action of the active compounds. Suitable cations are thus in particular the ions of the alkali metals, preferably sodium and potassium, of the alkaline earth metals, preferably calcium, magnesium and barium, of the transition metals, preferably manganese, copper, zinc and iron, and also the ammonium ion which, if desired, may carry 1 to 4 C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium. Anions of useful acid addition salts are primarily chloride, bromide, fluoride, hydrogensulfate, sulfate, dihydrogenphosphate, hydrogen-phosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate. They can be formed by reacting a compound I with an acid of the corresponding anion, preferably of hydrochloric acid, hydrobromic acid, sulfuric acid, phosphoric acid or nitric acid.

The scope of the present invention includes mixtures of the (R)- and (S)-isomers and the racemates of compounds I and/or II and/or III having one or more chiral centers. As a result of hindered rotation of asymmetrically substituted groups, atrope isomers of active compounds 1 and/or and/or III may be present. They also form part of the subject matter of the invention.

The active substances referred to as compounds II or III, their preparation and their activity e. g. against harmful fungi is known (cf.: http://www.alanwood.net/pesticides/); these substances are commercially available. The compounds described by IUPAC nomenclature, their preparation and their pesticidal activity are also known (cf. Can. J. Plant Sci. 48(6), 587-94, 1968; EP-A 141 317; EP-A 152 031; EP-A 226 917; EP-A 243 970; EP-A 256 503; EP-A 428 941; EP-A 532 022; EP-A 1 028 125; EP-A 1 035 122; EP-A 1 201 648; EP-A 1 122 244, JP 2002316902; DE 19650197; DE 10021412; DE 102005009458; U.S. Pat. Nos. 3,296,272; 3,325,503; WO 98/46608; WO 99/14187; WO 99/24413; WO 99/27783; WO 00/29404; WO 00/46148; WO 00/65913; WO 01/54501; WO 01/56358; WO 02/22583; WO 02/40431; WO 03/10149; WO 03/11853; WO 03/14103; WO 03/16286; WO 03/53145; WO 03/61388; WO 03/66609; WO 03/74491; WO 04/49804; WO 04/83193; WO 05/120234; WO 05/123689; WO 05/123690; WO 05/63721; WO 05/87772; WO 05/87773; WO 06/15866; WO 06/87325; WO 06/87343; WO 07/82098; WO 07/90624, WO 10/139271, WO 11/028657, WO 12/168188, WO 07/006670, WO 11/77514; WO 13/047749, WO 10/069882, WO 13/047441, WO 03/16303, WO 09/90181, WO 13/007767, WO 13/010862, WO 13/127704, WO 13/024009, WO 13/24010, WO 13/047441, WO 13/162072, WO 13/092224, WO 11/135833, CN 1907024, CN 1456054, CN 103387541, CN 1309897, WO 12/84812, CN 1907024, WO 09094442, WO 14/60177, WO 13/116251, WO 08/013622, WO 15/65922, WO 94/01546, EP 2865265, WO 07/129454, WO 12/165511, WO 11/081174, WO 13/47441).

Compounds of the formula I and their preparation and their use as fungicidally active compounds have been described in WO2015/185485 A1 and in the unpublished International applications WO 2017/076742 A1, WO 2017/081311 A1, WO 2017/085100 A1, WO 2017/093019 A1 and WO 2017/178245 A1.

Compounds of the formula I, wherein W contains a —C(═S)— group, can be prepared by reacting corresponding carboxamides with a sulfonation reagent, preferably Lawessons reagent (2,4-Bis-(4-methoxyphenyl)-1,3,2,4-dithiadiphosphetan-2,4-disulfid), in an organic solvent, preferably at temperatures between 20° C. and 150° C., preferably at 110° C., as previously described (see for example Journal of Organic Chemistry, 2008, 73, 9102 or European Journal of Organic Chemistry, 2015, 30, 6687).

In one embodiment the present invention relates to mixtures comprising as component 1) at least one active compound of the formula I, or an N-oxide, or an agriculturally useful salt thereof, wherein:
W is —(C═O)—NR²-#, —(C═S)—NR²-#, —NR²—(C═O)-# or —NR²—(C═S)-#; wherein # denotes the position, which is attached to the group R¹;
R¹ is alkoxyimino-C₁-C₄-alkyl;
R² is hydrogen, C₁-C₆-alkyl, C₂-C₆-alkenyl, C₂-C₆-alkynyl, C₁-C₆-alkoxy, C₃-C₈-cycloalkyl, C₃-C₅-cycloalkenyl, and C₃-C₅-cycloalkyl-C₁-C₄-alkyl; and wherein any of the aliphatic or cyclic groups are unsubstituted or substituted with 1, 2, 3 or up to the maximum possible number of identical or different radicals selected from the group consisting of halogen, hydroxy, oxo, cyano, C₁-C₆-alkyl, C₁-C₆-alkoxy and C₃-C₈-cycloalkyl;
m is 0 or 1;
R³, R⁴ independently of each other are selected from the group consisting of hydrogen, fluorine and methyl;
or R³ and R⁴ together with the carbon atom to which they are bound form a cyclopropyl ring.

In one embodiment the present invention relates to mixtures comprising as component 1) at least one active compound of the formula I, or an N-oxide, or an agriculturally useful salt thereof, wherein:
W is —NR²—(C═O)-# or —NR²—(C═S)-#; wherein # denotes the position, which is attached to the group R¹;
R¹ is C₁—C-alkyl or C₃-C₈-cycloalkyl;
R² is C₂-C₆-alkenyl or C₂-C₆-alkynyl; and wherein any of the aliphatic or cyclic groups are unsubstituted or substituted with 1, 2, 3 or up to the maximum possible number of identical or different radicals selected from the group consisting of halogen, hydroxy, oxo, cyano, C₁-C₆-alkyl, C₁-C₆-alkoxy and C₃-C₈-cycloalkyl;
m is 0 or 1;
R³, R⁴ independently of each other are selected from the group consisting of hydrogen, fluorine and methyl;
or R³ and R⁴ together with the carbon atom to which they are bound form a cyclopropyl ring.

In one embodiment the present invention relates to mixtures comprising as component 1) at least one active compound of the formula I, or an N-oxide, or an agriculturally useful salt thereof, wherein:
W is —NR²—(C═O)-# or —NR²—(C═S)-#; wherein # denotes the position, which is attached to the group R¹;
R¹ and R² together with the nitrogen atom to which they are bound form a saturated or partially unsaturated mono- or bicyclic 3- to 7-membered heterocycle, wherein the heterocycle includes beside one nitrogen atom and one or more carbon atoms no further heteroatom or 1, 2 or 3 further heteroatoms independently selected from N, O and S as ring member atoms; and wherein one or two CH₂ groups of the heterocycle may be replaced by one or two groups independently selected from the group of C(═O) and C(═S); and wherein the heterocycle is unsubstituted or carries 1, 2, 3, 4 or up to the maximum possible number of identical or different radicals selected from the group consisting of halogen, cyano, C1-C₆-alkyl, C₁-C₆-haloalkyl, C₁-C₆-alkoxy,
m is 1;
R³, R⁴ independently of each other are selected from the group consisting of hydrogen, fluorine and methyl;
or R³ and R⁴ together with the carbon atom to which they are bound form a cyclopropyl ring.

In one embodiment the present invention relates to mixtures comprising as component 1) at least one active compound of the formula I, or an N-oxide, or an agriculturally useful salt thereof, wherein:
W is —(C═O)—NR²-# or —(C═S)—NR²-#; wherein # denotes the position, which is attached to the group R¹;
m is 0;
R¹ is C₁-C₆-alkyl, C₃-C₈-cycloalkyl, C₃-C₈-cycloalkenyl, C₂-C₆-alkenyl, C₂-C₆-alkynyl or phenyl; wherein the phenyl ring is unsubstituted or substituted with 1, 2, 3 or up to the maximum possible number of identical or different radicals selected from the group consisting of halogen;
R² is hydrogen, C₁-C₆-alkyl, C₂-C₆-alkenyl, C₂-C₆-alkynyl, C₁-C₆-alkoxy, C₃-C₈-cycloalkyl, C₃-C₈-cycloalkenyl, and C₃-C₅-cycloalkyl-C₁-C₄-alkyl; and wherein any of the aliphatic or cyclic groups are unsubstituted or substituted with 1, 2, 3 or up to the maximum possible number of identical or different radicals selected from the group consisting of halogen, hydroxy, oxo, cyano, C₁-C₆-alkyl, C₁-C₆-alkoxy and C₃-C₈-cycloalkyl.

In one embodiment the present invention relates to mixtures comprising as component 1) at least one active compound of the formula I, or an N-oxide, or an agriculturally useful salt thereof, wherein:
W is —(C═O)—NR²-# or —(C═S)—NR²-#; wherein # denotes the position, which is attached to the group R¹;
m is 0;
R¹ is methyl, ethyl, n-propyl, iso-propyl, cyclopropyl or 2-fluorophenyl;
R² is hydrogen.

Particularly preferred active compounds I are selected from the group consisting of compounds I.A to I.N:

| | |
|---|---|
| I.A: | N-methyl-4-[5-(trifluoromethyl)-1,2,4-oxadiazol-3-yl]benzamide |
| I.B: | N-[(E)-methoxyiminomethyl]-4-[5-(trifluoromethyl)-1,2,4-oxadiazol-3-yl]benzamide |
| I.C: | N-[(Z)-methoxyiminomethyl]-4-[5-(trifluoromethyl)-1,2,4-oxadiazol-3-yl]benzamide |
| I.D: | N-[4-[5-(trifluoromethyl)-1,2,4-oxadiazol-3-yl]phenyl]cyclopropanecarboxamide |
| I.E: | N-(2-fluorophenyl)-4-[5-(trifluoromethyl)-1,2,4-oxadiazol-3-yl]benzamide |
| I.F: | 2,2-difluoro-N-methyl-2-[4-[5-(trifluoromethyl)-1,2,4-oxadiazol-3-yl]phenyl]acetamide |
| I.G: | N-allyl-N-[[4-[5-(trifluoromethyl)-1,2,4-oxadiazol-3-yl]phenyl]methyl]acetamide |
| I.H: | N-[(E)-N-methoxy-C-methyl-carbonimidoyl]-4-[5-(trifluoromethyl)-1,2,4-oxadiazol-3- |
| | yl]benzamide |
| I.J: | N-[(Z)-N-methoxy-C-methyl-carbonimidoyl]-4-[5-(trifluoromethyl)-1,2,4-oxadiazol-3- |
| | yl]benzamide |
| I.K: | N-allyl-N-[[4-[5-(trifluoromethyl)-1,2,4-oxadiazol-3-yl]phenyl]methyl]propanamide |
| I.L | 4,4-dimethyl-1-[[4-[5-(trifluoromethyl)-1,2,4-oxadiazol-3-yl]phenyl]methyl]pyrrolidin- |
| | 2-one |
| I.M | N-methyl-4-[5-(trifluoromethyl)-1,2,4-oxadiazol-3-yl]benzenecarbothioamide |
| I.N | 5-methyl-1-[[4-[5-(trifluoromethyl)-1,2,4-oxadiazol-3-yl]phenyl]methyl]pyrrolidin-2- |
| | one |

In one aspect of the invention active compounds I are selected from the group of compounds consisting of I.B, I.C, I.D, I.F, I.G, I.H, I.K, I.L and I.N.

Preference is also given to mixtures comprising as component 2) at least one active compound selected from inhibitors of complex III at Qₒ site in group A), more preferably selected from compounds (A.1.1), (A.1.4), (A.1.8), (A.1.9), (A.1.10), (A.1.11), (A.1.12), (A.1.13), (A.1.14), (A.1.17), (A.1.21), (A.1.21a), (A.1.24), (A.1.25), (A.1.26), (A.1.27), (A.1.30), (A.1.31), (A.1.32), (A.1.34) and (A.1.35); particularly selected from (A.1.1), (A.1.8), (A.1.9), (A.1.10), (A.1.11), (A.1.13), (A.1.14), (A.1.17), (A.1.25) and (A.1.34).

Preference is also given to mixtures comprising as component 2) at least one active compound selected from inhibitors of complex III at Qᵢ site in group A), more preferably selected from compounds (A.2.1), (A.2.3), (A.2.4), (A.2.5) and (A.2.6); particularly selected from (A.2.4) and (A.2.6).

Preference is also given to mixtures comprising as component 2) at least one active compound selected from inhibitors of complex II in group A), more preferably selected from compounds (A.3.2), (A.3.3), (A.3.4), (A.3.7), (A.3.9), (A.3.11), (A.3.12), (A.3.15), (A.3.16), (A.3.17), (A.3.18), (A.3.19), (A.3.20), (A.3.21), (A.3.22), (A.3.23), (A.3.24), (A.3.25), (A.3.26), (A.3.27), (A.3.28), (A.3.29), (A.3.31), (A.3.32), (A.3.33), (A.3.34), (A.3.35), (A.3.36), (A.3.37), (A.3.38) and (A.3.39); particularly selected from (A.3.2), (A.3.3), (A.3.4), (A.3.7), (A.3.9), (A.3.17), (A.3.24), (A.3.28), (A.3.29), (A.3.31), (A.3.32), (A.3.33), (A.3.34), (A.3.35), (A.3.36), (A.3.37), (A.3.38) and (A.3.39).

Preference is also given to mixtures comprising as component 2) at least one active compound selected from other respiration inhibitors in group A), more preferably selected from compounds (A.4.5) and (A.4.11).

Preference is also given to mixtures comprising as component 2) at least one active compound selected from C14 demethylase inhibitors in group B), more preferably selected from compounds (B.1.4), (B.1.5), (B.1.8), (B.1.10), (B.1.11), (B.1.12), (B.1.13), (B.1.17), (B.1.18), (B.1.21), (B.1.22), (B.1.23), (B.1.25), (B.1.26), (B.1.29), (B.1.33), (B.1.34), (B.1.37), (B.1.38), (B.1.43) and (B.1.46); particularly selected from (B.1.4), (B.1.5), (B.1.8), (B.1.17), (B.1.22), (B.1.23), (B.1.25), (B.1.29), and (B.1.38).

Preference is also given to mixtures comprising as component 2) at least one active compound selected from Delta14-reductase inhibitors in group B), more preferably selected from compounds (B.2.4), (B.2.5), (B.2.6) and (B.2.8); in particular (B.2.4) and (B.2.6).

Preference is also given to mixtures comprising as component 2) at least one active compound selected from phenylamides and acyl amino acid fungicides in group C), more preferably selected from compounds (C.1.1), (C.1.2), (C.1.4) and (C.1.5).

Preference is also given to mixtures comprising as component 2) at least one active compound selected from other nucleic acid synthesis inhibitors in group C), more preferably selected from compounds (C.2.6), (C.2.7) and (C.2.8).

Preference is also given to mixtures comprising as component 2) at least one active compound selected from group D), more preferably selected from compounds (D.1.1), (D.1.2), (D.1.5), (D.2.4), (D.2.6) and (D.2.7); particularly selected from (D.2.6) and (D.2.7).

Preference is also given to mixtures comprising as component 2) at least one active compound selected from group E), more preferably selected from compounds (E.1.1), (E.1.3), (E.2.2) and (E.2.3); in particular (E.1.1).

Preference is also given to mixtures comprising as component 2) at least one active compound selected from group F), more preferably selected from compounds (F.1.2), (F.1.4) and (F.1.5).

Preference is also given to mixtures comprising as component 2) at least one active compound selected from group G), more preferably selected from compounds (G.3.1), (G.3.3), (G.3.6), (G.5.1), (G.5.2), (G.5.3), (G.5.4), (G.5.5), G.5.6), G.5.7), (G.5.8), (G.5.9), (G.5.10) and (G.5.11).

Preference is also given to mixtures comprising as component 2) at least one active compound selected from group H), more preferably selected from compounds (H.1.5), (H.1.7), (H.2.2), (H.2.3), (H.2.5), (H.2.7), (H.2.8), (H.3.2), (H.3.4), (H.3.5), (H.4.9) and (H.4.10); particularly selected from (H.1.5), (H.1.7), (H.2.2), (H.3.2) and (H.3.5).

Preference is also given to mixtures comprising as component 2) at least one active compound selected from group 1), more preferably selected from compounds (I.2.2) and (I.2.5).

Preference is also given to mixtures comprising as component 2) at least one active compound selected from group J), more preferably selected from compounds (J.1.2), (J.1.5), (J.1.8), (J.1.11) and (J.1.12).

Preference is also given to mixtures comprising as component 2) at least one active compound selected from group K), more preferably selected from compounds (K.1.3), (K.1.29), (K.1.30), (K.1.31), (K.1.32), (K.1.41), (K.1.42), (K.1.44), (K.1.45), (K.1.47), (K.1.49) and (K.1.54); particularly selected from (K.1.3), (K.1.29), (K.1.30), (K.1.31), (K.1.32), (K.1.42), (K.1.53) and (K.1.54).

Particularly preferred are the following binary mixtures A-1 to A-739 listed in Table A, wherein compounds I (component 1, a group represented by the expression “(1)” consisting of compounds I.A, I.B, I.C, I.D, I.E, I.F, I.G, I.H, I.J, I.K, I.L, I.M and I.N) and one pesticide II selected from the groups A) to O) as defined herein (component 2, for example, (A.1.1) or azoxystrobin, in mixture A-1).

TABLE A
A-1: (I) + (A.1.1), A-2: (I) + (A.1.2), A-3: (I) + (A.1.3), A-4: (I) + (A.1.4), A-5: (I) + (A.1.5), A-6:
(I) + (A.1.6), A-7: (I) + (A.1.7), A-8: (I) + (A.1.8), A-9: (I) + (A.1.9), A-10: (I) + (A.1.10), A-11:
(I) + (A.1.11), A-12: (I) + (A.1.12), A-13: (I) + (A.1.13), A-14: (I) + (A.1.14), A-15: (I) + (A.1.15), A-16:
(I) + (A.1.16), A-17: (I) + (A.1.17), A-18: (I) + (A.1.18), A-19: (I) + (A.1.19), A-20: (I) + (A.1.20), A-21:
(I) + (A.1.21), A-22: (I) + (A.1.22), A-23: (I) + (A.1.23), A-24: (I) + (A.1.24), A-25: (I) + (A.1.25), A-26:
(I) + (A.1.26), A-27: (I) + (A.1.27), A-28: (I) + (A.1.30), A-29: (I) + (A.1.31), A-30: (I) + (A.1.32), A-31:
(I) + (A.2.1), A-32: (I) + (A.2.2), A-33: (I) + (A.2.3), A-34: (I) + (A.2.4), A-35: (I) + (A.2.6), A-36:
(I) + (A.2.7), A-37: (I) + (A.2.8), A-38: (I) + (A.3.1), A-39: (I) + (A.3.2), A-40: (I) + (A.3.3), A-41:
(I) + (A.3.4), A-42: (I) + (A.3.5), A-43: (I) + (A.3.6), A-44: (I) + (A.3.7), A-45: (I) + (A.3.8), A-46:
(I) + (A.3.9), A-47: (I) + (A.3.10), A-48: (I) + (A.3.11), A-49: (I) + (A.3.12), A-50: (I) + (A.3.13), A-51:
(I) + (A.3.14), A-52: (I) + (A.3.15), A-53: (I) + (A.3.16), A-54: (I) + (A.3.17), A-55: (I) + (A.3.18), A-56:
(I) + (A.3.19), A-57: (I) + (A.3.20), A-58: (I) + (A.3.21), A-59: (I) + (A.3.22), A-60: (I) + (A.3.23), A-61:
(I) + (A.3.24), A-62: (I) + (A.3.25), A-63: (I) + (A.3.26), A-64: (I) + (A.3.27), A-65: (I) + (A.3.28), A-66:
(I) + (A.3.29), A-67: (I) + (A.3.30), A-68: (I) + (A.3.31), A-69: (I) + (A.3.32), A-70: (I) + (A.3.33), A-71:
(I) + (A.3.34), A-72: (I) + (A.3.35), A-73: (I) + (A.3.36), A-74: (I) + (A.3.37), A-75: (I) + (A.3.38), A-76:
(I) + (A.3.39), A-77: (I) + (A.4.1), A-78: (I) + (A.4.2), A-79: (I) + (A.4.3), A-80: (I) + (A.4.4), A-81:
(I) + (A.4.5), A-82: (I) + (A.4.6), A-83: (I) + (A.4.7), A-84: (I) + (A.4.8), A-85: (I) + (A.4.9), A-86:
(I) + (A.4.10), A-87: (I) + (A.4.11), A-88: (I) + (A.4.12), A-89: (I) + (B.1.1), A-90: (I) + (B.1.2), A-91:
(I) + (B.1.3), A-92: (I) + (B.1.4), A-93: (I) + (B.1.5), A-94: (I) + (B.1.6), A-95: (I) + (B.1.7), A-96:
(I) + (B.1.8), A-97: (I) + (B.1.9), A-98: (I) + (B.1.10), A-99: (I) + (B.1.11), A-100: (I) + (B.1.12), A-101:
(I) + (B.1.13), A-102: (I) + (B.1.14), A-103: (I) + (B.1.15), A-104: (I) + (B.1.16), A-105: (I) + (B.1.17), A-
106: (I) + (B.1.18), A-107: (I) + (B.1.19), A-108: (I) + (B.1.20), A-109: (I) + (B. 1.21), A-110:
(I) + (B.1.22), A-111: (I) + (B.1.23), A-112: (I) + (B.1.24), A-113: (I) + (B.1.25), A-114: (I) + (B.1.26), A-
115: (I) + (B.1.27), A-116: (I) + (B.1.28), A-117: (I) + (B.1.29), A-118: (I) + (B.1.30), A-119:
(I) + (B.1.34), A-120: (I) + (B.1.37), A-121: (I) + (B.1.38), A-122: (I) + (B.1.43), A-123: (I) + (B.1.44), A-
124: (I) + (B.1.45), A-125: (I) + (B.1.46), A-126: (I) + (B.1.47), A-127: (I) + (B.1.48), A-128:
(I) + (B.1.49), A-129: (I) + (B.1.50), A-130: (I) + (B.1.51), A-131: (I) + (B.2.1), A-132: (I) + (B.2.2), A-
133: (I) + (B.2.3), A-141: (I) + (B.2.4), A-135: (I) + (B.2.5), A-136: (I) + (B.2.6), A-137: (I) + (B.2.7), A-
138: (I) + (B.2.8), A-139: (I) + (B.3.1), A-140: (I) + (C.1.1), A-141: (I) + (C.1.2), A-142: (I) + (C. 1.3), A-
143: (I) + (C.1.4), A-144: (I) + (C.1.5), A-145: (I) + (C.1.6), A-146: (I) + (C.1.7), A-147: (I) + (C.2.1), A-
148: (I) + (C.2.2), A-149: (I) + (C.2.3), A-150: (I) + (C.2.4), A-151: (I) + (C.2.5), A-152: (I) + (C.2.6), A-
153: (I) + (C.2.7), A-154: (I) + (D.1.1), A-155: (I) + (D.1.2), A-156: (I) + (D.1.3), A-157: (I) + (D. 1.4), A-
158: (I) + (D.1.5), A-159: (I) + (D.1.6), A-160: (I) + (D.2.1), A-161: (I) + (D.2.2), A-162: (I) + (D.2.3), A-
163: (I) + (D.2.4), A-164: (I) + (D.2.5), A-165: (I) + (D.2.6), A-166: (I) + (D.2.7), A-167: (I) + (E. 1.1), A-
168: (I) + (E.1.2), A-169: (I) + (E.1.3), A-170: (I) + (E.2.1), A-171: (I) + (E.2.2), A-172: (I) + (E.2.3), A-
173: (I) + (E.2.4), A-174: (I) + (E.2.5), A-175: (I) + (E.2.6), A-176: (I) + (E.2.7), A-177: (I) + (E.2.8), A-
178: (I) + (F.1.1), A-179: (I) + (F.1.2), A-180: (I) + (F.1.3), A-181: (I) + (F.1.4), A-182: (I) + (F.1.5), A-
183: (I) + (F.1.6), A-184: (I) + (F.2.1), A-185: (I) + (G.1.1), A-186: (I) + (G.1.2), A-187: (I) + (G.1.3), A-
188: (I) + (G.1.4), A-189: (I) + (G.2.1), A-190: (I) + (G.2.2), A-191: (I) + (G.2.3), A-192: (I) + (G.2.4), A-
193: (I) + (G.2.5), A-194: (I) + (G.2.6), A-195: (I) + (G.2.7), A-196: (I) + (G.3.1), A-197: (I) + (G.3.2), A-
198: (I) + (G.3.3), A-199: (I) + (G.3.4), A-200: (I) + (G.3.5), A-201: (I) + (G.3.6), A-202: (I) + (G.3.7), A-
203: (I) + (G.3.8), A-204: (I) + (G.4.1), A-205: (I) + (G.5.1), A-206: (I) + (G.5.2), A-207: (I) + (G.5.3), A-
208: (I) + (H.1.1), A-209: (I) + (H.1.2), A-210: (I) + (H.1.3), A-211: (I) + (H.1.4), A-212: (I) + (H.1.5), A-
213: (I) + (H.1.6), A-214: (I) + (H.2.1), A-215: (I) + (H.2.2), A-216: (I) + (H.2.3), A-217: (I) + (H.2.4), A-
218: (I) + (H.2.5), A-219: (I) + (H.2.6), A-220: (I) + (H.2.7), A-221: (I) + (H.2.8), A-222: (I) + (H.2.9), A-
223: (I) + (H.3.1), A-224: (I) + (H.3.2), A-225: (I) + (H.3.3), A-226: (I) + (H.3.4), A-227: (I) + (H.3.5), A-
228: (I) + (H.3.6), A-229: (I) + (H.3.7), A-230: (I) + (H.3.8), A-231: (I) + (H.3.9), A-232: (I) + (H.3.10), A-
233: (I) + (H.3.11), A-234: (I) + (H.4.1), A-235: (I) + (H.4.2), A-236: (I) + (H.4.3), A-237: (I) + (H.4.4), A-
238: (I) + (H.4.5), A-239: (I) + (H.4.6), A-240: (I) + (H.4.7), A-241: (I) + (H.4.8), A-242: (I) + (H.4.9), A-
243: (I) + (H.4.10), A-244: (I) + (I.1.1), A-245: (I) + (I.1.2), A-246: (I) + (I.2.1), A-247: (I) + (I.2.2), A-
248: (I) + (I.2.3), A-249: (I) + (I.2.4), A-250: (I) + (I.2.5), A-251: (I) + (J.1.1), A-252: (I) + (J.1.2), A-253:
(I) + (J.1.3), A-254: (I) + (J.1.4), A-255: (I) + (J.1.5), A-256: (I) + (J.1.6), A-257: (I) + (J.1.7), A-258:
(I) + (J.1.8), A-259: (I) + (J.1.9), A-260: (I) + (J.1.10), A-261: (I) + (J.1.11), A-262: (I) + (J.1.12), A-263:
(I) + (K.1.1), A-264: (I) + (K.1.2), A-265: (I) + (K.1.3), A-266: (I) + (K.1.4), A-267: (I) + (K. 1.5), A-268:
(I) + (K.1.6), A-269: (I) + (K.1.7), A-270: (I) + (K.1.8), A-271: (I) + (K.1.9), A-272: (I) + (K. 1.10), A-273:
(I) + (K.1.11), A-274: (I) + (K.1.12), A-275: (I) + (K.1.13), A-276: (I) + (K.1.14), A-277: (I) + (K. 1.15), A-
278: (I) + (K.1.16), A-279: (I) + (K.1.17), A-280: (I) + (K.1.18), A-281: (I) + (K.1.19), A-282:
(I) + (K.1.20), A-283: (I) + (K.1.21), A-284: (I) + (K.1.22), A-285: (I) + (K.1.23), A-286: (I) + (K.1.24), A-
287: (I) + (K.1.25), A-288: (I) + (K.1.26), A-289: (I) + (K.1.27), A-290: (I) + (K.1.28), A-291:
(I) + (K.1.29), A-292: (I) + (K.1.30), A-293: (I) + (K.1.31), A-294: (I) + (K.1.32), A-295: (I) + (K.1.33), A-
296: (I) + (K.1.34), A-297: (I) + (K.1.35), A-298: (I) + (K.1.36), A-299: (I) + (K.1.37), A-300:
(I) + (K.1.38), A-301: (I) + (K.1.39), A-302: (I) + (K.1.40), A-303: (I) + (K.1.41), A-304: (I) + (K.1.42), A-
305: (I) + (K.1.43), A-306: (I) + (K.1.44), A-307: (I) + (K.1.45), A-308: (I) + (K.1.47), A-309:
(I) + (M.1.1), A-310: (I) + (M.1.2), A-311: (I) + (M.1.3), A-312: (I) + (M.1.4), A-313: (I) + (M.1.5), A-314:
(I) + (M.1.6), A-315: (I) + (M.1.7), A-316: (I) + (M.1.8), A-317: (I) + (M.1.9), A-318: (I) + (M.1.10), A-
319: (I) + (M.1.11), A-320: (I) + (M.1.12), A-321: (I) + (M.1.13), A-322: (I) + (M.1.14), A-323:
(I) + (M.1.15), A-324: (I) + (M.1.16), A-325: (I) + (M.1.17), A-326: (I) + (M.1.18), A-327: (I) + (M.1.19),
A-328: (I) + (M.1.20), A-329: (I) + (M.1.21), A-330: (I) + (M.1.22), A-331: (I) + (M.1.23), A-332:
(I) + (M.1.24), A-333: (I) + (M.1.25), A-334: (I) + (M.1.26), A-335: (I) + (M.1.27), A-336: (I) + (M.1.28),
A-337: (I) + (M.1.29), A-338: (I) + (M.1.30), A-339: (I) + (M.1.31), A-340: (I) + (M.1.32), A-341:
(I) + (M.1.33), A-342: (I) + (M.1.34), A-343: (I) + (M.1.35), A-344: (I) + (M.1.36), A-345: (I) + (M.1.37),
A-346: (I) + (M.1.38), A-347: (I) + (M.1.39), A-348: (I) + (M.1.40), A-349: (I) + (M. 1.41), A-350:
(I) + (M.1.42), A-351: (I) + (M.1.43), A-352: (I) + (M.1.44), A-353: (I) + (M.1.45), A-354: (I) + (M.1.46),
A-355: (I) + (M.1.47), A-356: (I) + (M.1.48), A-357: (I) + (M.1.49), A-358: (I) + (M.1.50), A-359:
(I) + (N.1.1), A-360: (I) + (N.1.2), A-361: (I) + (N.1.3), A-362: (I) + (N.1.4), A-363: (I) + (N.1.5), A-364:
(I) + (N.2.1), A-365: (I) + (N.2.2), A-366: (I) + (N.2.3), A-367: (I) + (N.3.1), A-368: (I) + (N.3.2), A-369:
(I) + (N.3.3), A-370: (I) + (N.3.4), A-371: (I) + (N.4.1), A-372: (I) + (N.5.1), A-373: (I) + (N.6.1), A-374:
(I) + (N.6.2), A-375: (I) + (N.6.3), A-376: (I) + (N.6.4), A-377: (I) + (N.6.5), A-378: (I) + (N.7.1), A-379:
(I) + (N.7.2), A-380: (I) + (N.7.3), A-381: (I) + (N.8.1), A-382: (I) + (N.9.1), A-383: (I) + (N. 10.1), A-384:
(I) + (N.10.2), A-385: (I) + (N.10.3), A-386: (I) + (N.10.4), A-387: (I) + (N.10.5), A-388: (I) + (N.11.1), A-
389: (I) + (N.12.1), A-390: (I) + (N.12.2), A-391: (I) + (N.12.3), A-392: (I) + (N.12.4), A-393:
(I) + (N.13.1), A-394: (I) + (N.13.2), A-395: (I) + (N.13.3), A-396: (I) + (N.13.4), A-397: (I) + (N.13.5), A-
398: (I) + (N.13.6), A-399: (I) + (N.13.7), A-400: (I) + (N.13.8), A-401: (I) + (N.13.9), A-402:
(I) + (N.14.1), A-403: (I) + (N.14.2), A-404: (I) + (N.14.3), A-405: (I) + (N.15.1), A-406: (I) + (N.16.1), A-
407: (I) + (N.16.2), A-408: (I) + (N.17.1), A-409: (I) + (N.17.2), A-410: (I) + (N.17.3), A-411:
(I) + (N.17.4), A-412: (I) + (N.17.5), A-413: (I) + (N.17.6), A-414: (I) + (N.17.7), A-415: (I) + (N.17.8), A-
416: (I) + (N.17.9), A-417: (I) + (N.17.10), A-418: (I) + (N.17.11), A-419: (I) + (N.17.12), A-420:
(I) + (O.1.1), A-421: (I) + (O.1.2), A-422: (I) + (O.1.3), A-423: (I) + (O.1.4), A-424: (I) + (O.1.5), A-425:
(I) + (O.1.6), A-426: (I) + (O.1.7), A-427: (I) + (O.1.8), A-428: (I) + (O.1.9), A-429: (I) + (O.1.10), A-430:
(I) + (O.1.11), A-431: (I) + (O.1.12), A-432: (I) + (O.1.13), A-433: (I) + (O.1.14), A-434: (I) + (O.1.15),
A-435: (I) + (O.1.16), A-436: (I) + (O.1.17), A-437: (I) + (O.1.18), A-438: (I) + (O.1.19), A-439:
(I) + (O.1.20), A-440: (I) + (O.1.21), A-441: (I) + (O.1.22), A-442: (I) + (O.1.23), A-443: (I) + (O.1.24),
A-444: (I) + (O.1.25), A-445: (I) + (O.1.26), A-446: (I) + (O.1.27), A-447: (I) + (O.1.28), A-448:
(I) + (O.1.29), A-449: (I) + (O.1.30), A-450: (I) + (O.1.31), A-451: (I) + (O.1.32), A-452: (I) + (O.1.33),
A-453: (I) + (O.1.34), A-454: (I) + (O.1.35), A-455: (I) + (O.1.36), A-456: (I) + (O.1.37), A-457:
(I) + (O.1.38), A-458: (I) + (O.2.1), A-459: (I) + (O.2.2), A-460: (I) + (O.2.3), A-461: (I) + (O.2.4), A-462:
(I) + (O.2.5), A-463: (I) + (O.2.6), A-464: (I) + (O.2.7), A-465: (I) + (O.2.8), A-466: (I) + (O.2.9), A-467:
(I) + (O.2.10), A-468: (I) + (O.2.11), A-469: (I) + (O.2.12), A-470: (I) + (O.2.13), A-471: (I) + (O.2.14),
A-472: (I) + (O.2.15), A-473: (I) + (O.2.16), A-474: (I) + (O.3.1), A-475: (I) + (O.3.2), A-476:
(I) + (O.3.3), A-477: (I) + (O.3.4), A-478: (I) + (O.3.5), A-479: (I) + (O.3.6), A-480: (I) + (O.3.7), A-481:
(I) + (O.3.8), A-482: (I) + (O.3.9), A-483: (I) + (O.3.10), A-484: (I) + (O.3.11), A-485: (I) + (O.3.12), A-
486: (I) + (O.3.13), A-487: (I) + (O.3.14), A-488: (I) + (O.3.15), A-489: (I) + (O.3.16), A-490:
(I) + (O.3.17), A-491: (I) + (O.3.18), A-492: (I) + (O.3.19), A-493: (I) + (O.3.20), A-494: (I) + (O.3.21),
A-495: (I) + (O.3.22), A-496: (I) + (O.3.23), A-497: (I) + (O.3.24), A-498: (I) + (O.3.25), A-499:
(I) + (O.3.26), A-500: (I) + (O.3.27), A-501: (I) + (O.4.1), A-502: (I) + (O.4.2), A-503: (I) + (O.4.3), A-
504: (I) + (O.4.4), A-505: (I) + (O.4.5), A-506: (I) + (O.4.6), A-507: (I) + (O.4.7), A-508: (I) + (O.4.8), A-
509: (I) + (O.4.9), A-510: (I) + (O.4.10), A-511: (I) + (O.4.11), A-512: (I) + (O.4.12), A-513:
(I) + (O.4.13), A-514: (I) + (O.4.14), A-515: (I) + (O.4.15), A-516: (I) + (O.4.16), A-517: (I) + (O.4.17),
A-518: (I) + (O.4.18), A-519: (I) + (O.4.19), A-520: (I) + (O.4.20), A-521: (I) + (O.4.21), A-522:
(I) + (O.4.22), A-523: (I) + (O.4.23), A-524: (I) + (O.4.24), A-525: (I) + (O.5.1), A-526: (I) + (O.5.2), A-
527: (I) + (O.5.3), A-528: (I) + (O.5.4), A-529: (I) + (O.5.5), A-530: (I) + (O.5.6), A-531: (I) + (O.5.7), A-
532: (I) + (O.5.8), A-533: (I) + (O.5.9), A-534: (I) + (O.6.1), A-535: (I) + (O.6.2), A-536: (I) + (O.6.3), A-
537: (I) + (O.6.4), A-538: (I) + (O.6.5), A-539: (I) + (O.6.6), A-540: (I) + (O.6.7), A-541: (I) + (O.7.1), A-
542: (I) + (O.7.2), A-543: (I) + (O.7.3), A-544: (I) + (O.7.4), A-545: (I) + (O.7.5), A-546: (I) + (O.7.6), A-
547: (I) + (O.8.1), A-548: (I) + (O.8.2), A-549: (I) + (O.8.3), A-550: (I) + (O.8.4), A-551: (I) + (O.8.5), A-
552: (I) + (O.9.1), A-553: (I) + (O.9.2), A-554: (I) + (O.9.3), A-555: (I) + (O.10.1), A-556: (I) + (O.11.1),
A-557: (I) + (O.11.2), A-558: (I) + (O.11.3), A-559: (I) + (O.11.4), A-560: (I) + (O.12.1), A-561:
(I) + (O.13.1), A-562: (I) + (O.14.1), A-563: (I) + (O.14.2), A-564: (I) + (O.15.1), A-565: (I) + (O.15.2),
A-566: (I) + (O.15.3), A-567: (I) + (O.15.4), A-568: (I) + (O.15.5), A-569: (I) + (O.15.6), A-570:
(I) + (O.15.7), A-571: (I) + (O.15.8), A-572: (I) + (O.15.9), A-573: (I) + (O.15.10), A-574: (I) + (O.15.11),
A-575: (I) + (O.16.1), A-576: (I) + (O.16.2), A-577: (I) + (O.16.3), A-578: (I) + (O.16.4), A-579:
(I) + (O.16.5), A-580: (I) + (O.16.6), A-581: (I) + (O.17.1), A-582: (I) + (O.18.1), A-583: (I) + (O.18.2),
A-584: (I) + (O.18.3), A-585: (I) + (O.18.4), A-586: (I) + (O.18.5), A-587: (I) + (O.19.1), A-588:
(I) + (O.20.1), A-589: (I) + (O.20.2), A-590: (I) + (O.20.3), A-591: (I) + (O.21.1), A-592: (I) + (O.21.2),
A-593: (I) + (O.21.3), A-594: (I) + (O.21.4), A-595: (I) + (O.21.5), A-596: (I) + (O.21.6), A-597:
(I) + (O.21.7), A-598: (I) + (O.22.1), A-599: (I) + (O.22.2), A-600: (I) + (O.22.3), A-601: (I) + (O.22.4),
A-602: (I) + (O.23.1), A-603: (I) + (O.23.2), A-604: (I) + (O.23.3), A-605: (I) + (O.24.1), A-606:
(I) + (O.24.2), A-607: (I) + (O.24.3), A-608: (I) + (O.24.4), A-609: (I) + (O.24.5), A-610: (I) + (O.25.1),
A-611: (I) + (O.25.2), A-612: (I) + (O.26.1), A-613: (I) + (O.26.2), A-614: (I) + (O.26.3), A-615:
(I) + (O.26.4), A-616: (I) + (O.26.5), A-617: (I) + (O.26.6), A-618: (I) + (O.26.7), A-619: (I) + (O.26.8),
A-620: (I) + (O.26.9), A-621: (I) + (O.26.10), A-622: (I) + (O.26.11), A-623: (I) + (O.26.12), A-624:
(I) + (O.26.13), A-625: (I) + (O.26.14), A-626: (I) + (O.26.15), A-627: (I) + (O.26.16), A-628:
(I) + (O.26.17), A-629: (I) + (O.26.18), A-630: (I) + (O.27.1), A-631: (I) + (O.27.2), A-632: (I) + (O.27.3),
A-633: (I) + (O.27.4), A-634: (I) + (O.27.5), A-635: (I) + (O.27.6), A-636: (I) + (O.27.7), A-637:
(I) + (O.27.8), A-638: (I) + (O.27.9), A-639: (I) + (O.27.10), A-640: (I) + (O.27.11), A-641:
(I) + (O.27.12), A-642: (I) + (O.27.13), A-643: (I) + (O.27.14), A-644: (I) + (O.27.15), A-645:
(I) + (O.27.16), A-646: (I) + (O.27.17), A-647: (I) + (O.27.18), A-648: (I) + (O.27.19), A-649:
(I) + (O.27.20), A-650: (I) + (O.27.21), A-651: (I) + (O.27.22), A-652: (I) + (O.27.23), A-653:
(I) + (O.27.24), A-654: (I) + (O.27.25), A-655: (I) + (O.27.26), A-656: (I) + (O.27.27), A-657:
(I) + (O.27.28), A-658: (I) + (O.27.29), A-659: (I) + (O.27.30), A-660: (I) + (O.27.31), A-661:
(I) + (O.27.32), A-662: (I) + (O.27.33), A-663: (I) + (O.27.34), A-664: (I) + (O.27.35), A-665:
(I) + (O.27.36), A-666: (I) + (O.27.37), A-667: (I) + (O.27.38), A-668: (I) + (O.27.39), A-669:
(I) + (O.27.40), A-670: (I) + (O.27.41), A-671: (I) + (O.27.42), A-672: (I) + (O.27.43), A-673:
(I) + (O.27.44), A-674: (I) + (O.27.45), A-675: (I) + (O.27.46), A-676: (I) + (O.27.47), A-677:
(I) + (O.27.48), A-678: (I) + (O.27.49), A-679: (I) + (O.27.50), A-680: (I) + (O.27.51), A-681:
(I) + (O.27.52), A-682: (I) + (O.27.53), A-683: (I) + (O.27.54), A-684: (I) + (O.27.55), A-685:
(I) + (O.27.56), A-686: (I) + (O.27.57), A-687: (I) + (O.27.58), A-688: (I) + (O.27.59), A-689:
(I) + (O.27.60), A-690: (I) + (O.27.61), A-691: (I) + (O.27.62), A-692: (I) + (O.27.63), A-693:
(I) + (O.27.64), A-694: (I) + (O.27.65), A-695: (I) + (O.27.66), A-696: (I) + (O.27.67), A-697:
(I) + (O.27.68), A-698: (I) + (O.27.69), A-699: (I) + (O.27.70), A-700: (I) + (O.27.71), A-701:
(I) + (O.27.72), A-702: (I) + (O.27.73), A-703: (I) + (O.27.74), A-704: (I) + (O.27.75), A-705:
(I) + (O.27.76), A-706: (I) + (O.27.77), A-707: (I) + (O.27.78), A-708: (I) + (O.27.79), A-709:
(I) + (O.27.80), A-710: (I) + (O.27.81), A-711: (I) + (O.27.82), A-712: (I) + (O.27.83), A-713:
(I) + (O.27.84), A-714: (I) + (O.27.85), A-715: (I) + (O.27.86), A-716: (I) + (O.27.87), A-717:
(I) + (O.27.88), A-718: (I) + (O.27.89), A-719: (I) + (O.27.90), A-720: (I) + (O.27.91), A-721:
(I) + (O.27.92), A-722: (I) + (O.27.93), A-723: (I) + (O.27.94), A-724: (I) + (O.27.95), A-725:
(I) + (O.27.96), A-726: (I) + (O.27.97), A-727: (I) + (O.27.98), A-728: (I) + (O.27.99), A-729:
(I) + (O.27.100), A-730: (I) + (A.1.21a), A-731: (I) + (K.1.46), A-732: (I) + (K.1.48), A-733:
(I) + (K.1.49), A-734: (I) + (K.1.50), A-735: (I) + (K.1.51), A-736: (I) + (K.1.52), A-737: (I) + (K.1.53), A-
738: (I) + (K.1.54), A-739: (I) + (A.2.6).

In one embodiment the weight ratio of a component 1) to component 2) in mixtures A-1 to A-739 of Table B is between 100:1 and 1:100, preferably between 50:1 and 1:50, more preferably between 20:1 and 1:20, particularly between 5:1 and 1:5; for example 3:1, 1:1 or 1:3.

In one embodiment the weight ratio of component 1) to component 2) is between 100:1 and 1:100 in mixtures A-1 to A-739 of Table A.

In one embodiment the weight ratio of component 1) to component 2) is between 50:1 and 1:50 in mixtures A-1 to A-739 of Table A.

In one embodiment the weight ratio of component 1) to component 2) is between 20:1 and 1:20 in mixtures A-1 to A-739 of Table A.

In one embodiment the weight ratio of component 1) to component 2) is between 5:1 and 1:5 in mixtures A-1 to A-739 of Table A.

In one embodiment the weight ratio of component 1) to component 2) is 3:1 in mixtures A-1 to A-739 of Table A.

In one embodiment the weight ratio of component 1) to component 2) is 1:1 in mixtures A-1 to A-739 of Table A.

In one embodiment the weight ratio of component 1) to component 2) is 1:3 in mixtures A-1 to A-739 of Table A.

In a preferred embodiment the mixtures A-1 to A-739 of Table A and in particular when the weight ratios of component 1) to component 2) are as disclosed for these mixtures as herein before mentioned, are used against Phakopsora pachyrhizi and P. meibomiae on soybeans and/or against Puccinia triticina, P. striiformis, P. hordei, P. graminis or P. recondita on wheat, barley or rye.

Particularly preferred are the binary mixtures listed in Table B1 to Table B13, for example mixtures B1-1 to B1-161 in table B1, wherein component 1) is compound I.A, and wherein component 2) is selected from compounds II of groups A) to K), as defined herein.

TABLE B1
B1-1: I.A + (A.1.1), B1-2: I.A + (A.1.4), B1-3: I.A + (A.1.8), B1-4: I.A + (A.1.9), B1-5: I.A +
(A.1.10), B1-6: I.A + (A.1.11), B1-7: I.A + (A.1.12), B1-8: I.A + (A.1.13), B1-9: I.A + (A.1.14), B1-
10: I.A + (A.1.17), B1-11: I.A + (A.1.21), B1-12: I.A + (A.1.21a), B1-13: I.A + (A.1.24), B1-14: I.A +
(A.1.25), B1-15: I.A + (A.1.26), B1-16: I.A + (A.1.27), B1-17: I.A + (A.1.30), B1-18: I.A +
(A.1.31), B1-19: I.A + (A.1.32), B1-20: I.A + (A.1.34), B1-21: I.A + (A.1.35), B1-22: I.A + (A.2.1),
B1-23: I.A + (A.2.3), B1-24: I.A + (A.2.4), B1-25: I.A + (A.2.5), B1-26: I.A + (A.2.6), B1-27: I.A +
(A.3.2), B1-28: I.A + (A.3.3), B1-29: I.A + (A.3.4), B1-30: I.A + (A.3.7), B1-31: I.A + (A.3.9), B1-
32: I.A + (A.3.11), B1-33: I.A + (A.3.12), B1-34: I.A + (A.3.15), B1-35: I.A + (A.3.16), B1-36: I.A +
(A.3.17), B1-37: I.A + (A.3.18), B1-38: I.A + (A.3.19), B1-39: I.A + (A.3.20), B1-40: I.A +
(A.3.21), B1-41: I.A + (A.3.22), B1-42: I.A + (A.3.23), B1-43: I.A + (A.3.24), B1-44: I.A +
(A.3.25), B1-45: I.A + (A.3.26), B1-46: I.A + (A.3.27), B1-47: I.A + (A.3.28), B1-48: I.A +
(A.3.29), B1-49: I.A + (A.3.31), B1-50: I.A + (A.3.32), B1-51: I.A + (A.3.33), B1-52: I.A +
(A.3.34), B1-53: I.A + (A.3.35), B1-54: I.A + (A.3.36), B1-55: I.A + (A.3.37), B1-56: I.A +
(A.3.38), B1-57: I.A + (A.3.39), B1-58: I.A + (A.4.5), B1-59: I.A + (A.4.11), B1-60: I.A + (B.1.4),
B1-61: I.A + (B.1.5), B1-62: I.A + (B.1.8.), B1-63: I.A + (B.1.10), B1-64: I.A + (B.1.11), B1-65: I.A +
(B.1.12), B1-66: I.A + (B.1.13), B1-67: I.A + (B.1.17), B1-68: I.A + (B.1.18), B1-69: I.A +
(B.1.21), B1-70: I.A + (B.1.22), B1-71: I.A + (B.1.23), B1-72: I.A + (B.1.25), B1-73: I.A +
(B.1.26), B1-74: I.A + (B.1.29), B1-75: I.A + (B.1.33), B1-76: I.A + (B.1.34), B1-77: I.A +
(B.1.37), B1-78: I.A + (B.1.38), B1-79: I.A + (B.1.43), B1-80: I.A + (B.1.46), B1-81: I.A + (B.2.4),
B1-82: I.A + (B.2.5), B1-83: I.A + (B.2.6), B1-84: I.A + (B.2.8), B1-85: I.A + (C.1.1), B1-86: I.A +
(C.1.2), B1-87: I.A + (C.1.4), B1-88: I.A + (C.1.5), B1-89: I.A + (C.2.6), B1-90: I.A + (C.2.7), B1-
91: I.A + (C.2.8), B1-92: I.A + (D.1.1), B1-93: I.A + (D.1.2), B1-94: I.A + (D.1.5), B1-95: I.A +
(D.2.4), B1-96: I.A + (D.2.6), B1-97: I.A + (D.2.7), B1-98: I.A + (E.1.1), B1-99: I.A + (E.1.3), B1-
100: I.A + (E.2.2), B1-101: I.A + (E.2.3), B1-102: I.A + (F.1.2), B1-103: I.A + (F.1.4), B1-104: I.A +
(F.1.5), B1-105: I.A + (G.3.1), B1-106: I.A + (G.3.3), B1-107: I.A + (G.3.6), B1-108: I.A +
(G.5.1), B1-109: I.A + (G.5.2), B1-110: I.A + (G.5.3), B1-111: I.A + (G.5.4), B1-112: I.A +
(G.5.5), B1-113: I.A + (G.5.6), B1-114: I.A + (G.5.7), B1-115: I.A + (G.5.8), B1-116: I.A +
(G.5.9), B1-117: I.A + (G.5.10), B1-118: I.A + (G.5.11), B1-119: I.A + (H.1.5), B1-120: I.A +
(H.1.7), B1-121: I.A + (H.2.2), B1-122: I.A + (H.2.3), B1-123: I.A + (H.2.5), B1-124: I.A + (H.2.7),
B1-125: I.A + (H.2.8), B1-126: I.A + (H.3.2), B1-127: I.A + (H.3.4), B1-128: I.A + (H.3.5), B1-
129: I.A + (H.4.9), B1-130: I.A + (H.4.10), B1-131: I.A + (I.2.2), B1-132: I.A + (I.2.5), B1-133: I.A +
(J.1.2), B1-134: I.A + (J.1.5), B1-135: I.A + (J.1.8), B1-136: I.A + (J.1.11), B1-137: I.A +
(J.1.12), B1-138: I.A + (K.1.3), B1-139: I.A + (K.1.29), B1-140: I.A + (K.1.30), B1-141: I.A +
(K.1.31), B1-142: I.A + (K.1.32), B1-143: I.A + (K.1.41), B1-144: I.A + (K.1.42), B1-145: I.A +
(K.1.44), B1-146: I.A + (K.1.45), B1-147: I.A + (K.1.47), B1-148: I.A + (K.1.49), B1-149: I.A +
(K.1.53), B1-150: I.A + (K.1.54), B1-151: I.A + (O.4.2), B1-152: I.A + (O.4.5), B1-153: I.A +
(O.4.8), B1-154: I.A + (O.26.2), B1-155: I.A + (O.26.3), B1-156: I.A + (O.26.4), B1-157: I.A +
(O.26.5), B1-158: I.A + (O.27.7), B1-159: I.A + (O.27.76), B1-160: I.A + (O.27.78), B1-161: I.A +
(O.27.79).

In one embodiment the weight ratio of compound I.A to component 2) in mixtures B1-1 to B1-161 of Table B1 is between 100:1 and 1:100, preferably between 50:1 and 1:50, more preferably between 20:1 and 1:20, particularly between 5:1 and 1:5; for example 3:1, 1:1 or 1:3.

Table B2: Mixtures B2-1 to B2-161 are defined as mixtures B1-1 to B1-161 of Table B1, wherein compound I.A in each mixture is replaced with compound I.B.

Table B3: Mixtures B3-1 to B3-161 are defined as mixtures B1-1 to B1-161 of Table B1, wherein compound I.A in each mixture is replaced with compound I.C.

Table B4: Mixtures B4-1 to B4-161 are defined as mixtures B1-1 to B1-161 of Table B1, wherein compound I.A in each mixture is replaced with compound I.D.

Table B5: Mixtures B5-1 to B5-161 are defined as mixtures B1-1 to B1-161 of Table B1, wherein compound I.A in each mixture is replaced with compound I.E.

Table B6: Mixtures B6-1 to B6-161 are defined as mixtures B1-1 to B1-161 of Table B1, wherein compound I.A in each mixture is replaced with compound I.F.

Table B7: Mixtures B7-1 to B7-161 are defined as mixtures B1-1 to B1-161 of Table B1, wherein compound I.A in each mixture is replaced with compound I.G.

Table B8: Mixtures B8-1 to B8-161 are defined as mixtures B1-1 to B1-161 of Table B1, wherein compound I.A in each mixture is replaced with compound I.H.

Table B9: Mixtures B9-1 to B9-161 are defined as mixtures B1-1 to B1-161 of Table B1, wherein compound I.A in each mixture is replaced with compound I.J.

Table B10: Mixtures B10-1 to B10-161 are defined as mixtures B1-1 to B1-161 of Table B1, wherein compound I.A in each mixture is replaced with compound I.K.

Table B11: Mixtures B11-1 to B11-161 are defined as mixtures B1-1 to B1-161 of Table B1, wherein compound I.A in each mixture is replaced with compound I.L.

Table B12: Mixtures B12-1 to B12-161 are defined as mixtures B1-1 to B1-161 of Table B1, wherein compound I.A in each mixture is replaced with compound I.M.

Table B13: Mixtures B13-1 to B13-161 are defined as mixtures B1-1 to B1-161 of Table B1, wherein compound I.A in each mixture is replaced with compound I.N.

In one embodiment the weight ratio of component 1) to component 2) is between 100:1 and 1:100 in mixtures as defined in Tables B1 to B13.

In one embodiment the weight ratio of component 1) to component 2) is between 50:1 and 1:50 in mixtures as defined in Tables B1 to B13.

In one embodiment the weight ratio of component 1) to component 2) is between 20:1 and 1:20 in mixtures as defined in Tables B1 to B13.

In one embodiment the weight ratio of component 1) to component 2) is between 5:1 and 1:5 in mixtures as defined in Tables B1 to B13.

In one embodiment the weight ratio of component 1) to component 2) is 3:1 in mixtures as defined in Tables B1 to B13.

In one embodiment the weight ratio of component 1) to component 2) is 1:1 in mixtures as defined in Tables B1 to B13.

In one embodiment the weight ratio of component 1) to component 2) is 1:3 in mixtures as defined in Tables B1 to B13.

In a preferred embodiment the mixtures as defined in Tables to B13 and in particular when the weight ratios of compound I to component 2) are as disclosed for these mixtures as herein before mentioned, are used against Phakopsora pachyrhizi and P. meibomiae on soybeans and/or against Puccinia triticina, P. striiformis, P. hordei, P. graminis or P. recondita on wheat, barley or rye.

Further preferred embodiments E.1 to E.144 listed in Table B14 relate to binary mixtures as defined in Tables B1 to B13 in particular weight ratios of component 1) to component 2).

TABLE B14

| | | Weight ratio |
|---|---|---|
| | | component 1) to |
| Embodiment | Mixture | component 2) |
| E.1 | B1-14 | 100:1 to 1:100 |
| E.2 | B1-31 | 100:1 to 1:100 |
| E.3 | B1-71 | 100:1 to 1:100 |
| E.4 | B1-78 | 100:1 to 1:100 |
| E.5 | B2-14 | 100:1 to 1:100 |
| E.6 | B2-31 | 100:1 to 1:100 |
| E.7 | B2-71 | 100:1 to 1:100 |
| E.8 | B2-78 | 100:1 to 1:100 |
| E.9 | B3-14 | 100:1 to 1:100 |
| E.10 | B3-31 | 100:1 to 1:100 |
| E.11 | B3-71 | 100:1 to 1:100 |
| E.12 | B3-78 | 100:1 to 1:100 |
| E.13 | B4-14 | 100:1 to 1:100 |
| E.14 | B4-31 | 100:1 to 1:100 |
| E.15 | B4-71 | 100:1 to 1:100 |
| E.16 | B4-78 | 100:1 to 1:100 |
| E.17 | B5-14 | 100:1 to 1:100 |
| E.18 | B5-31 | 100:1 to 1:100 |
| E.19 | B5-71 | 100:1 to 1:100 |
| E.20 | B5-78 | 100:1 to 1:100 |
| E.21 | B6-14 | 100:1 to 1:100 |
| E.22 | B6-31 | 100:1 to 1:100 |
| E.23 | B6-71 | 100:1 to 1:100 |
| E.24 | B6-78 | 100:1 to 1:100 |
| E.25 | B1-14 | 50:1 to 1:50 |
| E.26 | B1-31 | 50:1 to 1:50 |
| E.27 | B1-71 | 50:1 to 1:50 |
| E.28 | B1-78 | 50:1 to 1:50 |
| E.29 | B2-14 | 50:1 to 1:50 |
| E.30 | B2-31 | 50:1 to 1:50 |
| E.31 | B2-71 | 50:1 to 1:50 |
| E.32 | B2-78 | 50:1 to 1:50 |
| E.33 | B3-14 | 50:1 to 1:50 |
| E.34 | B3-31 | 50:1 to 1:50 |
| E.35 | B3-71 | 50:1 to 1:50 |
| E.36 | B3-78 | 50:1 to 1:50 |
| E.37 | B4-14 | 50:1 to 1:50 |
| E.38 | B4-31 | 50:1 to 1:50 |
| E.39 | B4-71 | 50:1 to 1:50 |
| E.40 | B4-78 | 50:1 to 1:50 |
| E.41 | B5-14 | 50:1 to 1:50 |
| E.42 | B5-31 | 50:1 to 1:50 |
| E.43 | B5-71 | 50:1 to 1:50 |
| E.44 | B5-78 | 50:1 to 1:50 |
| E.45 | B6-14 | 50:1 to 1:50 |
| E.46 | B6-31 | 50:1 to 1:50 |
| E.47 | B6-71 | 50:1 to 1:50 |
| E.48 | B6-78 | 50:1 to 1:50 |
| E.49 | B1-14 | 20:1 to 1:20 |
| E.50 | B1-31 | 20:1 to 1:20 |
| E.51 | B1-71 | 20:1 to 1:20 |
| E.52 | B1-78 | 20:1 to 1:20 |
| E.53 | B2-14 | 20:1 to 1:20 |
| E.54 | B2-31 | 20:1 to 1:20 |
| E.55 | B2-71 | 20:1 to 1:20 |
| E.56 | B2-78 | 20:1 to 1:20 |
| E.57 | B3-14 | 20:1 to 1:20 |
| E.58 | B3-31 | 20:1 to 1:20 |
| E.59 | B3-71 | 20:1 to 1:20 |
| E.60 | B3-78 | 20:1 to 1:20 |
| E.61 | B4-14 | 20:1 to 1:20 |
| E.62 | B4-31 | 20:1 to 1:20 |
| E.63 | B4-71 | 20:1 to 1:20 |
| E.64 | B4-78 | 20:1 to 1:20 |
| E.65 | B5-14 | 20:1 to 1:20 |
| E.66 | B5-31 | 20:1 to 1:20 |
| E.67 | B5-71 | 20:1 to 1:20 |
| E.68 | B5-78 | 20:1 to 1:20 |
| E.69 | B6-14 | 20:1 to 1:20 |
| E.70 | B6-31 | 20:1 to 1:20 |
| E.71 | B6-71 | 20:1 to 1:20 |
| E.72 | B6-78 | 20:1 to 1:20 |
| E.73 | B1-14 | 5:1 to 1:5 |
| E.74 | B1-31 | 5:1 to 1:5 |
| E.75 | B1-71 | 5:1 to 1:5 |
| E.76 | B1-78 | 5:1 to 1:5 |
| E.77 | B2-14 | 5:1 to 1:5 |
| E.78 | B2-31 | 5:1 to 1:5 |
| E.79 | B2-71 | 5:1 to 1:5 |
| E.80 | B2-78 | 5:1 to 1:5 |
| E.81 | B3-14 | 5:1 to 1:5 |
| E.82 | B3-31 | 5:1 to 1:5 |
| E.83 | B3-71 | 5:1 to 1:5 |
| E.84 | B3-78 | 5:1 to 1:5 |
| E.85 | B4-14 | 5:1 to 1:5 |
| E.86 | B4-31 | 5:1 to 1:5 |
| E.87 | B4-71 | 5:1 to 1:5 |
| E.88 | B4-78 | 5:1 to 1:5 |
| E.89 | B5-14 | 5:1 to 1:5 |
| E.90 | B5-31 | 5:1 to 1:5 |
| E.91 | B5-71 | 5:1 to 1:5 |
| E.92 | B5-78 | 5:1 to 1:5 |
| E.93 | B6-14 | 5:1 to 1:5 |
| E.94 | B6-31 | 5:1 to 1:5 |
| E.95 | B6-71 | 5:1 to 1:5 |
| E.96 | B6-78 | 5:1 to 1:5 |
| E.97 | B1-14 | 3:1 to 1:3 |
| E.98 | B1-31 | 3:1 to 1:3 |
| E.99 | B1-71 | 3:1 to 1:3 |
| E.100 | B1-78 | 3:1 to 1:3 |
| E.101 | B2-14 | 3:1 to 1:3 |
| E.102 | B2-31 | 3:1 to 1:3 |
| E.103 | B2-71 | 3:1 to 1:3 |
| E.104 | B2-78 | 3:1 to 1:3 |
| E.105 | B3-14 | 3:1 to 1:3 |
| E.106 | B3-31 | 3:1 to 1:3 |
| E.107 | B3-71 | 3:1 to 1:3 |
| E.108 | B3-78 | 3:1 to 1:3 |
| E.109 | B4-14 | 3:1 to 1:3 |
| E.110 | B4-31 | 3:1 to 1:3 |
| E.111 | B4-71 | 3:1 to 1:3 |
| E.112 | B4-78 | 3:1 to 1:3 |
| E.113 | B5-14 | 3:1 to 1:3 |
| E.114 | B5-31 | 3:1 to 1:3 |
| E.115 | B5-71 | 3:1 to 1:3 |
| E.116 | B5-78 | 3:1 to 1:3 |
| E.117 | B6-14 | 3:1 to 1:3 |
| E.118 | B6-31 | 3:1 to 1:3 |
| E.119 | B6-71 | 3:1 to 1:3 |
| E.120 | B6-78 | 3:1 to 1:3 |
| E.121 | B1-14 | 1:1 |
| E.122 | B1-31 | 1:1 |
| E.123 | B1-71 | 1:1 |
| E.124 | B1-78 | 1:1 |
| E.125 | B2-14 | 1:1 |
| E.126 | B2-31 | 1:1 |
| E.127 | B2-71 | 1:1 |
| E.128 | B2-78 | 1:1 |
| E.129 | B3-14 | 1:1 |
| E.130 | B3-31 | 1:1 |
| E.131 | B3-71 | 1:1 |
| E.132 | B3-78 | 1:1 |
| E.133 | B4-14 | 1:1 |
| E.134 | B4-31 | 1:1 |
| E.135 | B4-71 | 1:1 |
| E.136 | B4-78 | 1:1 |
| E.137 | B5-14 | 1:1 |
| E.138 | B5-31 | 1:1 |
| E.139 | B5-71 | 1:1 |
| E.140 | B5-78 | 1:1 |
| E.141 | B6-14 | 1:1 |
| E.142 | B6-31 | 1:1 |
| E.143 | B6-71 | 1:1 |
| E.144 | B6-78 | 1:1 |

A preferred embodiment relates to the use of the mixtures according to embodiments E.1 to E.144 of Table B14 against Phakopsora pachyrhizi and P. meibomiae on soybeans and/or against Puccinia triticina, P. striiformis, P. hordei, P. graminis or P. recondita on wheat, barley or rye.

It may be preferred that the binary mixtures above comprise besides at least one compound I and at least one compound as component 3) at least one further active compound III, resulting in a ternary mixture. Preferably, the components 1) and 2), or, all three components 1), 2) and 3), in these mixtures are present in a synergistically effective amount.

Accordingly one embodiment relates to ternary mixtures comprising, as active components
1) at least one active compound I as defined or preferably defined herein, or an N-oxide, or an agriculturally useful salt thereof;
   and as component
2) at least one active compound selected from groups A) to O) as defined for component 2) in binary mixtures above, or an N-oxide, or an agriculturally useful salt thereof; and as component
3) at least one active compound III selected from groups A) to O) as defined for component 2) in binary mixtures above, or an N-oxide, or an agriculturally useful salt thereof;
   wherein the at least one active compound III of component 3) is not identical with the at least one active compound II of component 2).

The invention also relates to a method for controlling phytopathogenic harmful fungi using the abovementioned ternary mixtures; to agrochemical compositions comprising these ternary mixtures; and to agrochemical compositions further comprising seed comprising these mixtures.

It is preferred that the ternary mixtures comprise as compounds III fungicidal compounds that are independently of each other selected from the groups A), B), C), D), E), F), G), H), I), J), K) and O); and wherein the at least one active compound III of component 3) is not identical with the at least one active compound II of component 2).

Preference is also given to ternary mixtures based on binary mixtures disclosed herein, comprising as component 3) at least one active compound selected from inhibitors of complex III at Qₒ site in group A), more preferably selected from compounds (A.1.1), (A.1.4), (A.1.8), (A.1.9), (A.1.10), (A.1.11), (A.1.12), (A.1.13), (A.1.14), (A.1.17), (A.1.21), (A.1.21a), (A.1.24), (A.1.25), (A.1.26), (A.1.27), (A.1.30), (A.1.31), (A.1.32), (A.1.34) and (A.1.35); particularly selected from (A.1.1), (A.1.8), (A.1.9), (A.1.10), (A.1.11), (A.1.13), (A.1.14), (A.1.17), (A.1.25), and (A.1.34); and wherein the at least one active compound III of component 3) is not identical with the at least one active compound II of component 2).

Preference is also given to ternary mixtures based on binary mixtures disclosed herein, comprising as component 3) at least one active compound selected from inhibitors of complex III at Qᵢ site in group A), more preferably selected from compounds (A.2.1), (A.2.3), (A.2.4), (A.2.5) and (A.2.6); particularly selected from (A.2.4) and (A.2.6); and wherein the at least one active compound III of component 3) is not identical with the at least one active compound II of component 2).

Preference is also given to ternary mixtures based on binary mixtures disclosed herein, comprising as component 3) at least one active compound selected from inhibitors of complex II in group A), more preferably selected from compounds (A.3.2), (A.3.3), (A.3.4), (A.3.7), (A.3.9), (A.3.11), (A.3.12), (A.3.15), (A.3.16), (A.3.17), (A.3.18), (A.3.19), (A.3.20), (A.3.21), (A.3.22), (A.3.23), (A.3.24), (A.3.25), (A.3.26), (A.3.27), (A.3.28), (A.3.29), (A.3.31), (A.3.32), (A.3.33), (A.3.34), (A.3.35), (A.3.36), (A.3.37), (A.3.38) and (A.3.39); particularly selected from (A.3.2), (A.3.3), (A.3.4), (A.3.7), (A.3.9), (A.3.17), (A.3.24), (A.3.28), (A.3.29), (A.3.31), (A.3.32), (A.3.33), (A.3.34), (A.3.35), (A.3.36), (A.3.37), (A.3.38) and (A.3.39); and wherein the at least one active compound III of component 3) is not identical with the at least one active compound II of component 2).

Preference is also given to ternary mixtures based on binary mixtures disclosed herein, comprising as component 3) at least one active compound selected from other respiration inhibitors in group A), more preferably selected from compounds (A.4.5) and (A.4.11); and wherein the at least one active compound III of component 3) is not identical with the at least one active compound II of component 2).

Preference is also given to ternary mixtures based on binary mixtures disclosed herein, comprising as component 3) at least one active compound selected from C14 demethylase inhibitors in group B), more preferably selected from compounds (B.1.4), (B.1.5), (B.1.8), (B.1.10), (B.1.11), (B.1.12), (B.1.13), (B.1.17), (B.1.18), (B.1.21), (B.1.22), (B.1.23), (B.1.25), (B.1.26), (B.1.29), (B.1.33), (B.1.34), (B.1.37), (B.1.38), (B.1.43) and (B.1.46); particularly selected from (B.1.4), (B.1.5), (B.1.8), (B.1.17), (B.1.22), (B.1.23), (B.1.25), (B.1.29), and (B.1.38); and wherein the at least one active compound III of component 3) is not identical with the at least one active compound II of component 2).

Preference is also given to ternary mixtures based on binary mixtures disclosed herein, comprising as component 3) at least one active compound selected from Delta14-reductase inhibitors in group B), more preferably selected from compounds (B.2.4), (B.2.5), (B.2.6) and (B.2.8); in particular (B.2.4) and (B.2.6); and wherein the at least one active compound III of component 3) is not identical with the at least one active compound II of component 2).

Preference is also given to ternary mixtures based on binary mixtures disclosed herein, comprising as component 3) at least one active compound selected from phenylamides and acyl amino acid fungicides in group C), more preferably selected from compounds (C.1.1), (C.1.2), (C.1.4) and (C.1.5); and wherein the at least one active compound III of component 3) is not identical with the at least one active compound II of component 2).

Preference is also given to ternary mixtures based on binary mixtures disclosed herein, comprising as component 3) at least one active compound selected from other nucleic acid synthesis inhibitors in group C), more preferably selected from compounds (C.2.6), (C.2.7) and (C.2.8); and wherein the at least one active compound III of component 3) is not identical with the at least one active compound II of component 2).

Preference is also given to ternary mixtures based on binary mixtures disclosed herein, comprising as component 3) at least one active compound selected from group D), more preferably selected from compounds (D.1.1), (D.1.2), (D.1.5), (D.2.4), (D.2.6) and (D.2.7); particularly selected from (D.2.6) and (D.2.7); and wherein the at least one active compound Ill of component 3) is not identical with the at least one active compound II of component 2).

Preference is also given to ternary mixtures based on binary mixtures disclosed herein, comprising as component 3) at least one active compound selected from group E), more preferably selected from compounds (E.1.1), (E.1.3), (E.2.2) and (E.2.3); in particular (E.1.1); and wherein the at least one active compound III of component 3) is not identical with the at least one active compound II of component 2).

Preference is also given to ternary mixtures based on binary mixtures disclosed herein, comprising as component 3) at least one active compound selected from group F), more preferably selected from compounds (F.1.2), (F.1.4) and (F.1.5); and wherein the at least one active compound III of component 3) is not identical with the at least one active compound II of component 2).

Preference is also given to ternary mixtures based on binary mixtures disclosed herein, comprising as component 3) at least one active compound selected from group G), more preferably selected from compounds (G.3.1), (G.3.3), (G.3.6), (G.5.1), (G.5.2), (G.5.3), (G.5.4), (G.5.5), (G.5.6), G.5.7), (G.5.8), (G.5.9), (G.5.10) and (G.5.11); and wherein the at least one active compound III of component 3) is not identical with the at least one active compound II of component 2).

Preference is also given to ternary mixtures based on binary mixtures disclosed herein, comprising as component 3) at least one active compound selected from group H), more preferably selected from compounds (H.1.5), (H.1.7), (H.2.2), (H.2.3), (H.2.5), (H.2.7), (H.2.8), (H.3.2), (H.3.4), (H.3.5), (H.4.9) and (H.4.10); particularly selected from (H.1.5), (H.1.7), (H.2.2), (H.3.2) and (H.3.5); and wherein the at least one active compound III of component 3) is not identical with the at least one active compound II of component 2).

Preference is also given to ternary mixtures based on binary mixtures disclosed herein, comprising as component 3) at least one active compound selected from group 1), more preferably selected from compounds (I.2.2) and (I.2.5); and wherein the at least one active compound III of component 3) is not identical with the at least one active compound II of component 2).

Preference is also given to ternary mixtures based on binary mixtures disclosed herein, comprising as component 3) at least one active compound selected from group J), more preferably selected from compounds (J.1.2), (J.1.5), (J.1.8), (J.1.11) and (J.1.12); and wherein the at least one active compound III of component 3) is not identical with the at least one active compound II of component 2).

Preference is also given to ternary mixtures based on binary mixtures disclosed herein, comprising as component 3) at least one active compound selected from group K), more preferably selected from compounds (K.1.3), (K.1.29), (K.1.30), (K.1.31), (K.1.32), (K.1.41), (K.1.42), (K.1.44), (K.1.45), (K.1.47), (K.1.49), (K.1.53) and (K.1.54); particularly selected from (K.1.3), (K.1.29), (K.1.30), (K.1.31), (K.1.32), (K.1.42), (K.1.53) and (K.1.54); and wherein the at least one active compound III of component 3) is not identical with the at least one active compound II of component 2).

The present invention relates furthermore to the mixtures as defined in Tables C1 to C6 comprising as component 1) one of the compounds I.A, I.B, I.C, I.D, I.E, I.F, I.G, I.H, I.J, I.K, I.L, I.M or I.N as defined above, and as component 2) one of the compounds as defined and numbered above from groups A) to K), and as component 3) a compound III from groups A) to K) as defined herein. Preferably, the compositions comprise the active substances in synergistically effective amounts.

TABLE C1
Ternary mixtures C1-1 to C1-6865 comprising compound I.A as defined herein as component 1)
and one compound II as defined above as component 2) and one compound III as defined above
as component 3). The components 1), 2) and 3) are written down in this order (comp. 1) +
comp. 2) + comp. 3)), so that one mixture, for example mixture C1-1, comprises as component
1) compound I.A, as component 2) compound (A.1.4) and as component 3) compound (A.1.1).

C1-1: I.A + (A.1.4) + (A.1.1), C1-2: I.A + (A.1.8) + (A.1.1), C1-3: I.A + (A.1.9) + (A.1.1), C1-4: I.A +
(A.1.10) + (A.1.1), C1-5: I.A + (A.1.11) + (A.1.1), C1-6: I.A + (A.1.12) + (A.1.1), C1-7: I.A +
(A.1.13) + (A.1.1), C1-8: I.A + (A.1.14) + (A.1.1), C1-9: I.A + (A.1.17) + (A.1.1), C1-10: I.A +
(A.1.21) + (A.1.1), C1-11: I.A + (A.1.21a) + (A.1.1), C1-12: I.A + (A.1.24) + (A.1.1), C1-13: I.A +
(A.1.25) + (A.1.1), C1-14: I.A + (A.1.26) + (A.1.1), C1-15: I.A + (A.1.27) + (A.1.1), C1-16: I.A +
(A.1.30) + (A.1.1), C1-17: I.A + (A.1.31) + (A.1.1), C1-18: I.A + (A.1.32) + (A.1.1), C1-19: I.A +
(A.1.34) + (A.1.1), C1-20: I.A + (A.1.35) + (A.1.1), C1-21: I.A + (A.2.1) + (A.1.1), C1-22: I.A +
(A.2.3) + (A.1.1), C1-23: I.A + (A.2.4) + (A.1.1), C1-24: I.A + (A.2.5) + (A.1.1), C1-25: I.A +
(A.2.6) + (A.1.1), C1-26: I.A + (A.3.2) + (A.1.1), C1-27: I.A + (A.3.3) + (A.1.1), C1-28: I.A +
(A.3.4) + (A.1.1), C1-29: I.A + (A.3.7) + (A.1.1), C1-30: I.A + (A.3.9) + (A.1.1), C1-31: I.A +
(A.3.11) + (A.1.1), C1-32: I.A + (A.3.12) + (A.1.1), C1-33: I.A + (A.3.15) + (A.1.1), C1-34: I.A +
(A.3.16) + (A.1.1), C1-35: I.A + (A.3.17) + (A.1.1), C1-36: I.A + (A.3.18) + (A.1.1), C1-37: I.A +
(A.3.19) + (A.1.1), C1-38: I.A + (A.3.20) + (A.1.1), C1-39: I.A + (A.3.21) + (A.1.1), C1-40: I.A +
(A.3.22) + (A.1.1), C1-41: I.A + (A.3.23) + (A.1.1), C1-42: I.A + (A.3.24) + (A.1.1), C1-43: I.A +
(A.3.25) + (A.1.1), C1-44: I.A + (A.3.26) + (A.1.1), C1-45: I.A + (A.3.27) + (A.1.1), C1-46: I.A +
(A.3.28) + (A.1.1), C1-47: I.A + (A.3.29) + (A.1.1), C1-48: I.A + (A.3.31) + (A.1.1), C1-49: I.A +
(A.3.32) + (A.1.1), C1-50: I.A + (A.3.33) + (A.1.1), C1-51: I.A + (A.3.34) + (A.1.1), C1-52: I.A +
(A.3.35) + (A.1.1), C1-53: I.A + (A.3.36) + (A.1.1), C1-54: I.A + (A.3.37) + (A.1.1), C1-55: I.A +
(A.3.38) + (A.1.1), C1-56: I.A + (A.3.39) + (A.1.1), C1-57: I.A + (A.4.5) + (A.1.1), C1-58: I.A +
(A.4.11) + (A.1.1), C1-59: I.A + (B.1.4) + (A.1.1), C1-60: I.A + (B.1.5) + (A.1.1), C1-61: I.A +
(B.1.8.) + (A.1.1), C1-62: I.A + (B.1.10) + (A.1.1), C1-63: I.A + (B.1.11) + (A.1.1), C1-64: I.A +
(B.1.12) + (A.1.1), C1-65: I.A + (B.1.13) + (A.1.1), C1-66: I.A + (B.1.17) + (A.1.1), C1-67: I.A +
(B.1.18) + (A.1.1), C1-68: I.A + (B.1.21) + (A.1.1), C1-69: I.A + (B.1.22) + (A.1.1), C1-70: I.A +
(B.1.23) + (A.1.1), C1-71: I.A + (B.1.25) + (A.1.1), C1-72: I.A + (B.1.26) + (A.1.1), C1-73: I.A +
(B.1.29) + (A.1.1), C1-74: I.A + (B.1.33) + (A.1.1), C1-75: I.A + (B.1.34) + (A.1.1), C1-76: I.A +
(B.1.37) + (A.1.1), C1-77: I.A + (B.1.38) + (A.1.1), C1-78: I.A + (B.1.43) + (A.1.1), C1-79: I.A +
(B.1.46) + (A.1.1), C1-80: I.A + (B.2.4) + (A.1.1), C1-81: I.A + (B.2.5) + (A.1.1), C1-82: I.A +
(B.2.6) + (A.1.1), C1-83: I.A + (B.2.8) + (A.1.1), C1-84: I.A + (C.1.1) + (A.1.1), C1-85: I.A +
(C.1.2) + (A.1.1), C1-86: I.A + (C.1.4) + (A.1.1), C1-87: I.A + (C.1.5) + (A.1.1), C1-88: I.A +
(C.2.6) + (A.1.1), C1-89: I.A + (C.2.7) + (A.1.1), C1-90: I.A + (C.2.8) + (A.1.1), C1-91: I.A +
(D.1.1) + (A.1.1), C1-92: I.A + (D.1.2) + (A.1.1), C1-93: I.A + (D.1.5) + (A.1.1), C1-94: I.A +
(D.2.4) + (A.1.1), C1-95: I.A + (D.2.6) + (A.1.1), C1-96: I.A + (D.2.7) + (A.1.1), C1-97: I.A +
(E.1.1) + (A.1.1), C1-98: I.A + (E.1.3) + (A.1.1), C1-99: I.A + (E.2.2) + (A.1.1), C1-100: I.A +
(E.2.3) + (A.1.1), C1-101: I.A + (F.1.2) + (A.1.1), C1-102: I.A + (F.1.4) + (A.1.1), C1-103: I.A +
(F.1.5) + (A.1.1), C1-104: I.A + (G.3.1) + (A.1.1), C1-105: I.A + (G.3.3) + (A.1.1), C1-106: I.A +
(G.3.6) + (A.1.1), C1-107: I.A + (G.5.1) + (A.1.1), C1-108: I.A + (G.5.2) + (A.1.1), C1-109: I.A +
(G.5.3) + (A.1.1), C1-110: I.A + (G.5.4) + (A.1.1), C1-111: I.A + (G.5.5) + (A.1.1), C1-112: I.A +
(G.5.6) + (A.1.1), C1-113: I.A + (G.5.7) + (A.1.1), C1-114: I.A + (G.5.8) + (A.1.1), C1-115: I.A +
(G.5.9) + (A.1.1), C1-116: I.A + (G.5.10) + (A.1.1), C1-117: I.A + (G.5.11) + (A.1.1), C1-118: I.A +
(H.1.5) + (A.1.1), C1-119: I.A + (H.1.7) + (A.1.1), C1-120: I.A + (H.2.2) + (A.1.1), C1-121: I.A +
(H.2.3) + (A.1.1), C1-122: I.A + (H.2.5) + (A.1.1), C1-123: I.A + (H.2.7) + (A.1.1), C1-124: I.A +
(H.2.8) + (A.1.1), C1-125: I.A + (H.3.2) + (A.1.1), C1-126: I.A + (H.3.4) + (A.1.1), C1-127: I.A +
(H.3.5) + (A.1.1), C1-128: I.A + (H.4.9) + (A.1.1), C1-129: I.A + (H.4.10) + (A.1.1), C1-130: I.A +
(I.2.2) + (A.1.1), C1-131: I.A + (I.2.5) + (A.1.1), C1-132: I.A + (J.1.2) + (A.1.1), C1-133: I.A +
(J.1.5) + (A.1.1), C1-134: I.A + (J.1.8) + (A.1.1), C1-135: I.A + (J.1.11) + (A.1.1), C1-136: I.A +
(J.1.12) + (A.1.1), C1-137: I.A + (K.1.3) + (A.1.1), C1-138: I.A + (K.1.29) + (A.1.1), C1-139: I.A +
(K.1.30) + (A.1.1), C1-140: I.A + (K.1.31) + (A.1.1), C1-141: I.A + (K.1.32) + (A.1.1), C1-142: I.A +
(K.1.41) + (A.1.1), C1-143: I.A + (K.1.42) + (A.1.1), C1-144: I.A + (K.1.44) + (A.1.1), C1-145:
I.A + (K.1.45) + (A.1.1), C1-146: I.A + (K.1.47) + (A.1.1), C1-147: I.A + (K.1.49) + (A.1.1), C1-
148: I.A + (K.1.53) + (A.1.1), C1-149: I.A + (K.1.54) + (A.1.1), C1-150: I.A + (A.1.4) + (A.1.8),
C1-151: I.A + (A.1.9) + (A.1.8), C1-152: I.A + (A.1.10) + (A.1.8), C1-153: I.A + (A.1.11) +
(A.1.8), C1-154: I.A + (A.1.12) + (A.1.8), C1-155: I.A + (A.1.13) + (A.1.8), C1-156: I.A + (A.1.14) +
(A.1.8), C1-157: I.A + (A.1.17) + (A.1.8), C1-158: I.A + (A.1.21) + (A.1.8), C1-159: I.A +
(A.1.21a) + (A.1.8), C1-160: I.A + (A.1.24) + (A.1.8), C1-161: I.A + (A.1.25) + (A.1.8), C1-162:
I.A + (A.1.26) + (A.1.8), C1-163: I.A + (A.1.27) + (A.1.8), C1-164: I.A + (A.1.30) + (A.1.8), C1-
165: I.A + (A.1.31) + (A.1.8), C1-166: I.A + (A.1.32) + (A.1.8), C1-167: I.A + (A.1.34) + (A.1.8),
C1-168: I.A + (A.1.35) + (A.1.8), C1-169: I.A + (A.2.1) + (A.1.8), C1-170: I.A + (A.2.3) + (A.1.8),
C1-171: I.A + (A.2.4) + (A.1.8), C1-172: I.A + (A.2.5) + (A.1.8), C1-173: I.A + (A.2.6) + (A.1.8),
C1-174: I.A + (A.3.2) + (A.1.8), C1-175: I.A + (A.3.3) + (A.1.8), C1-176: I.A + (A.3.4) + (A.1.8),
C1-177: I.A + (A.3.7) + (A.1.8), C1-178: I.A + (A.3.9) + (A.1.8), C1-179: I.A + (A.3.11) + (A.1.8),
C1-180: I.A + (A.3.12) + (A.1.8), C1-181: I.A + (A.3.15) + (A.1.8), C1-182: I.A + (A.3.16) +
(A.1.8), C1-183: I.A + (A.3.17) + (A.1.8), C1-184: I.A + (A.3.18) + (A.1.8), C1-185: I.A + (A.3.19) +
(A.1.8), C1-186: I.A + (A.3.20) + (A.1.8), C1-187: I.A + (A.3.21) + (A.1.8), C1-188: I.A +
(A.3.22) + (A.1.8), C1-189: I.A + (A.3.23) + (A.1.8), C1-190: I.A + (A.3.24) + (A.1.8), C1-191: I.A +
(A.3.25) + (A.1.8), C1-192: I.A + (A.3.26) + (A.1.8), C1-193: I.A + (A.3.27) + (A.1.8), C1-194:
I.A + (A.3.28) + (A.1.8), C1-195: I.A + (A.3.29) + (A.1.8), C1-196: I.A + (A.3.31) + (A.1.8), C1-
197: I.A + (A.3.32) + (A.1.8), C1-198: I.A + (A.3.33) + (A.1.8), C1-199: I.A + (A.3.34) + (A.1.8),
C1-200: I.A + (A.3.35) + (A.1.8), C1-201: I.A + (A.3.36) + (A.1.8), C1-202: I.A + (A.3.37) +
(A.1.8), C1-203: I.A + (A.3.38) + (A.1.8), C1-204: I.A + (A.3.39) + (A.1.8), C1-205: I.A + (A.4.5) +
(A.1.8), C1-206: I.A + (A.4.11) + (A.1.8), C1-207: I.A + (B.1.4) + (A.1.8), C1-208: I.A + (B.1.5) +
(A.1.8), C1-209: I.A + (B.1.8.) + (A.1.8), C1-210: I.A + (B.1.10) + (A.1.8), C1-211: I.A +
(B.1.11) + (A.1.8), C1-212: I.A + (B.1.12) + (A.1.8), C1-213: I.A + (B.1.13) + (A.1.8), C1-214: I.A +
(B.1.17) + (A.1.8), C1-215: I.A + (B.1.18) + (A.1.8), C1-216: I.A + (B.1.21) + (A.1.8), C1-217:
I.A + (B.1.22) + (A.1.8), C1-218: I.A + (B.1.23) + (A.1.8), C1-219: I.A + (B.1.25) + (A.1.8), C1-
220: I.A + (B.1.26) + (A.1.8), C1-221: I.A + (B.1.29) + (A.1.8), C1-222: I.A + (B.1.33) + (A.1.8),
C1-223: I.A + (B.1.34) + (A.1.8), C1-224: I.A + (B.1.37) + (A.1.8), C1-225: I.A + (B.1.38) +
(A.1.8), C1-226: I.A + (B.1.43) + (A.1.8), C1-227: I.A + (B.1.46) + (A.1.8), C1-228: I.A + (B.2.4) +
(A.1.8), C1-229: I.A + (B.2.5) + (A.1.8), C1-230: I.A + (B.2.6) + (A.1.8), C1-231: I.A + (B.2.8) +
(A.1.8), C1-232: I.A + (C.1.1) + (A.1.8), C1-233: I.A + (C.1.2) + (A.1.8), C1-234: I.A + (C.1.4) +
(A.1.8), C1-235: I.A + (C.1.5) + (A.1.8), C1-236: I.A + (C.2.6) + (A.1.8), C1-237: I.A + (C.2.7) +
(A.1.8), C1-238: I.A + (C.2.8) + (A.1.8), C1-239: I.A + (D.1.1) + (A.1.8), C1-240: I.A + (D.1.2) +
(A.1.8), C1-241: I.A + (D.1.5) + (A.1.8), C1-242: I.A + (D.2.4) + (A.1.8), C1-243: I.A + (D.2.6) +
(A.1.8), C1-244: I.A + (D.2.7) + (A.1.8), C1-245: I.A + (E.1.1) + (A.1.8), C1-246: I.A + (E.1.3) +
(A.1.8), C1-247: I.A + (E.2.2) + (A.1.8), C1-248: I.A + (E.2.3) + (A.1.8), C1-249: I.A + (F.1.2) +
(A.1.8), C1-250: I.A + (F.1.4) + (A.1.8), C1-251: I.A + (F.1.5) + (A.1.8), C1-252: I.A + (G.3.1) +
(A.1.8), C1-253: I.A + (G.3.3) + (A.1.8), C1-254: I.A + (G.3.6) + (A.1.8), C1-255: I.A + (G.5.1) +
(A.1.8), C1-256: I.A + (G.5.2) + (A.1.8), C1-257: I.A + (G.5.3) + (A.1.8), C1-258: I.A + (G.5.4) +
(A.1.8), C1-259: I.A + (G.5.5) + (A.1.8), C1-260: I.A + (G.5.6) + (A.1.8), C1-261: I.A + (G.5.7) +
(A.1.8), C1-262: I.A + (G.5.8) + (A.1.8), C1-263: I.A + (G.5.9) + (A.1.8), C1-264: I.A + (G.5.10) +
(A.1.8), C1-265: I.A + (G.5.11) + (A.1.8), C1-266: I.A + (H.1.5) + (A.1.8), C1-267: I.A + (H.1.7) +
(A.1.8), C1-268: I.A + (H.2.2) + (A.1.8), C1-269: I.A + (H.2.3) + (A.1.8), C1-270: I.A + (H.2.5) +
(A.1.8), C1-271: I.A + (H.2.7) + (A.1.8), C1-272: I.A + (H.2.8) + (A.1.8), C1-273: I.A + (H.3.2) +
(A.1.8), C1-274: I.A + (H.3.4) + (A.1.8), C1-275: I.A + (H.3.5) + (A.1.8), C1-276: I.A + (H.4.9) +
(A.1.8), C1-277: I.A + (H.4.10) + (A.1.8), C1-278: I.A + (I.2.2) + (A.1.8), C1-279: I.A + (I.2.5) +
(A.1.8), C1-280: I.A + (J.1.2) + (A.1.8), C1-281: I.A + (J.1.5) + (A.1.8), C1-282: I.A + (J.1.8) +
(A.1.8), C1-283: I.A + (J.1.11) + (A.1.8), C1-284: I.A + (J.1.12) + (A.1.8), C1-285: I.A + (K.1.3) +
(A.1.8), C1-286: I.A + (K.1.29) + (A.1.8), C1-287: I.A + (K.1.30) + (A.1.8), C1-288: I.A + (K.1.31) +
(A.1.8), C1-289: I.A + (K.1.32) + (A.1.8), C1-290: I.A + (K.1.41) + (A.1.8), C1-291: I.A +
(K.1.42) + (A.1.8), C1-292: I.A + (K.1.44) + (A.1.8), C1-293: I.A + (K.1.45) + (A.1.8), C1-294: I.A +
(K.1.47) + (A.1.8), C1-295: I.A + (K.1.49) + (A.1.8), C1-296: I.A + (K.1.53) + (A.1.8), C1-297:
I.A + (K.1.54) + (A.1.8), C1-298: I.A + (A.1.4) + (A.1.9), C1-299: I.A + (A.1.10) + (A.1.9), C1-
300: I.A + (A.1.11) + (A.1.9), C1-301: I.A + (A.1.12) + (A.1.9), C1-302: I.A + (A.1.13) + (A.1.9),
C1-303: I.A + (A.1.14) + (A.1.9), C1-304: I.A + (A.1.17) + (A.1.9), C1-305: I.A + (A.1.21) +
(A.1.9), C1-306: I.A + (A.1,21a) + (A.1.9), C1-307: I.A + (A.1.24) + (A.1.9), C1-308: I.A +
(A.1.25) + (A.1.9), C1-309: I.A + (A.1.26) + (A.1.9), C1-310: I.A + (A.1.27) + (A.1.9), C1-311: I.A +
(A.1.30) + (A.1.9), C1-312: I.A + (A.1.31) + (A.1.9), C1-313: I.A + (A.1.32) + (A.1.9), C1-314:
I.A + (A.1.34) + (A.1.9), C1-315: I.A + (A.1.35) + (A.1.9), C1-316: I.A + (A.2.1) + (A.1.9), C1-
317: I.A + (A.2.3) + (A.1.9), C1-318: I.A + (A.2.4) + (A.1.9), C1-319: I.A + (A.2.5) + (A.1.9), C1-
320: I.A + (A.2.6) + (A.1.9), C1-321: I.A + (A.3.2) + (A.1.9), C1-322: I.A + (A.3.3) + (A.1.9), C1-
323: I.A + (A.3.4) + (A.1.9), C1-324: I.A + (A.3.7) + (A.1.9), C1-325: I.A + (A.3.9) + (A.1.9), C1-
326: I.A + (A.3.11) + (A.1.9), C1-327: I.A + (A.3.12) + (A.1.9), C1-328: I.A + (A.3.15) + (A.1.9),
C1-329: I.A + (A.3.16) + (A.1.9), C1-330: I.A + (A.3.17) + (A.1.9), C1-331: I.A + (A.3.18) +
(A.1.9), C1-332: I.A + (A.3.19) + (A.1.9), C1-333: I.A + (A.3.20) + (A.1.9), C1-334: I.A + (A.3.21) +
(A.1.9), C1-335: I.A + (A.3.22) + (A.1.9), C1-336: I.A + (A.3.23) + (A.1.9), C1-337: I.A +
(A.3.24) + (A.1.9), C1-338: I.A + (A.3.25) + (A.1.9), C1-339: I.A + (A.3.26) + (A.1.9), C1-340: I.A +
(A.3.27) + (A.1.9), C1-341: I.A + (A.3.28) + (A.1.9), C1-342: I.A + (A.3.29) + (A.1.9), C1-343:
I.A + (A.3.31) + (A.1.9), C1-344: I.A + (A.3.32) + (A.1.9), C1-345: I.A + (A.3.33) + (A.1.9), C1-
346: I.A + (A.3.34) + (A.1.9), C1-347: I.A + (A.3.35) + (A.1.9), C1-348: I.A + (A.3.36) + (A.1.9),
C1-349: I.A + (A.3.37) + (A.1.9), C1-350: I.A + (A.3.38) + (A.1.9), C1-351: I.A + (A.3.39) +
(A.1.9), C1-352: I.A + (A.4.5) + (A.1.9), C1-353: I.A + (A.4.11) + (A.1.9), C1-354: I.A + (B.1.4) +
(A.1.9), C1-355: I.A + (B.1.5) + (A.1.9), C1-356: I.A + (B.1.8.) + (A.1.9), C1-357: I.A + (B.1.10) +
(A.1.9), C1-358: I.A + (B.1.11) + (A.1.9), C1-359: I.A + (B.1.12) + (A.1.9), C1-360: I.A + (B.1.13) +
(A.1.9), C1-361: I.A + (B.1.17) + (A.1.9), C1-362: I.A + (B.1.18) + (A.1.9), C1-363: I.A +
(B.1.21) + (A.1.9), C1-364: I.A + (B.1.22) + (A.1.9), C1-365: I.A + (B.1.23) + (A.1.9), C1-366: I.A +
(B.1.25) + (A.1.9), C1-367: I.A + (B.1.26) + (A.1.9), C1-368: I.A + (B.1.29) + (A.1.9), C1-369:
I.A + (B.1.33) + (A.1.9), C1-370: I.A + (B.1.34) + (A.1.9), C1-371: I.A + (B.1.37) + (A.1.9), C1-
372: I.A + (B.1.38) + (A.1.9), C1-373: I.A + (B.1.43) + (A.1.9), C1-374: I.A + (B.1.46) + (A.1.9),
C1-375: I.A + (B.2.4) + (A.1.9), C1-376: I.A + (B.2.5) + (A.1.9), C1-377: I.A + (B.2.6) + (A.1.9),
C1-378: I.A + (B.2.8) + (A.1.9), C1-379: I.A + (C.1.1) + (A.1.9), C1-380: I.A + (C.1.2) + (A.1.9),
C1-381: I.A + (C.1.4) + (A.1.9), C1-382: I.A + (C.1.5) + (A.1.9), C1-383: I.A + (C.2.6) + (A.1.9),
C1-384: I.A + (C.2.7) + (A.1.9), C1-385: I.A + (C.2.8) + (A.1.9), C1-386: I.A + (D.1.1) + (A.1.9),
C1-387: I.A + (D.1.2) + (A.1.9), C1-388: I.A + (D.1.5) + (A.1.9), C1-389: I.A + (D.2.4) + (A.1.9),
C1-390: I.A + (D.2.6) + (A.1.9), C1-391: I.A + (D.2.7) + (A.1.9), C1-392: I.A + (E.1.1) + (A.1.9),
C1-393: I.A + (E.1.3) + (A.1.9), C1-394: I.A + (E.2.2) + (A.1.9), C1-395: I.A + (E.2.3) + (A.1.9),
C1-396: I.A + (F.1.2) + (A.1.9), C1-397: I.A + (F.1.4) + (A.1.9), C1-398: I.A + (F.1.5) + (A.1.9),
C1-399: I.A + (G.3.1) + (A.1.9), C1-400: I.A + (G.3.3) + (A.1.9), C1-401: I.A + (G.3.6) + (A.1.9),
C1-402: I.A + (G.5.1) + (A.1.9), C1-403: I.A + (G.5.2) + (A.1.9), C1-404: I.A + (G.5.3) + (A.1.9),
C1-405: I.A + (G.5.4) + (A.1.9), C1-406: I.A + (G.5.5) + (A.1.9), C1-407: I.A + (G.5.6) + (A.1.9),
C1-408: I.A + (G.5.7) + (A.1.9), C1-409: I.A + (G.5.8) + (A.1.9), C1-410: I.A + (G.5.9) + (A.1.9),
C1-411: I.A + (G.5.10) + (A.1.9), C1-412: I.A + (G.5.11) + (A.1.9), C1-413: I.A + (H.1.5) +
(A.1.9), C1-414: I.A + (H.1.7) + (A.1.9), C1-415: I.A + (H.2.2) + (A.1.9), C1-416: I.A + (H.2.3) +
(A.1.9), C1-417: I.A + (H.2.5) + (A.1.9), C1-418: I.A + (H.2.7) + (A.1.9), C1-419: I.A + (H.2.8) +
(A.1.9), C1-420: I.A + (H.3.2) + (A.1.9), C1-421: I.A + (H.3.4) + (A.1.9), C1-422: I.A + (H.3.5) +
(A.1.9), C1-423: I.A + (H.4.9) + (A.1.9), C1-424: I.A + (H.4.10) + (A.1.9), C1-425: I.A + (I.2.2) +
(A.1.9), C1-426: I.A + (I.2.5) + (A.1.9), C1-427: I.A + (J.1.2) + (A.1.9), C1-428: I.A + (J.1.5) +
(A.1.9), C1-429: I.A + (J.1.8) + (A.1.9), C1-430: I.A + (J.1.11) + (A.1.9), C1-431: I.A + (J.1.12) +
(A.1.9), C1-432: I.A + (K.1.3) + (A.1.9), C1-433: I.A + (K.1.29) + (A.1.9), C1-434: I.A + (K.1.30) +
(A.1.9), C1-435: I.A + (K.1.31) + (A.1.9), C1-436: I.A + (K.1.32) + (A.1.9), C1-437: I.A +
(K.1.41) + (A.1.9), C1-438: I.A + (K.1.42) + (A.1.9), C1-439: I.A + (K.1.44) + (A.1.9), C1-440: I.A +
(K.1.45) + (A.1.9), C1-441: I.A + (K.1.47) + (A.1.9), C1-442: I.A + (K.1.49) + (A.1.9), C1-443:
I.A + (K.1.53) + (A.1.9), C1-444: I.A + (K.1.54) + (A.1.9), C1-445: I.A + (A.1.4) + (A.1.10), C1-
446: I.A + (A.1.11) + (A.1.10), C1-447: I.A + (A.1.12) + (A.1.10), C1-448: I.A + (A.1.13) +
(A.1.10), C1-449: I.A + (A.1.14) + (A.1.10), C1-450: I.A + (A.1.17) + (A.1.10), C1-451: I.A +
(A.1.21) + (A.1.10), C1-452: I.A + (A.1.21a) + (A.1.10), C1-453: I.A + (A.1.24) + (A.1.10), C1-
454: I.A + (A.1.25) + (A.1.10), C1-455: I.A + (A.1.26) + (A.1.10), C1-456: I.A + (A.1.27) +
(A.1.10), C1-457: I.A + (A.1.30) + (A.1.10), C1-458: I.A + (A.1.31) + (A.1.10), C1-459: I.A +
(A.1.32) + (A.1.10), C1-460: I.A + (A.1.34) + (A.1.10), C1-461: I.A + (A.1.35) + (A.1.10), C1-462:
I.A + (A.2.1) + (A.1.10), C1-463: I.A + (A.2.3) + (A.1.10), C1-464: I.A + (A.2.4) + (A.1.10), C1-
465: I.A + (A.2.5) + (A.1.10), C1-466: I.A + (A.2.6) + (A.1.10), C1-467: I.A + (A.3.2) + (A.1.10),
C1-468: I.A + (A.3.3) + (A.1.10), C1-469: I.A + (A.3.4) + (A.1.10), C1-470: I.A + (A.3.7) +
(A.1.10), C1-471: I.A + (A.3.9) + (A.1.10), C1-472: I.A + (A.3.11) + (A.1.10), C1-473: I.A +
(A.3.12) + (A.1.10), C1-474: I.A + (A.3.15) + (A.1.10), C1-475: I.A + (A.3.16) + (A.1.10), C1-476:
I.A + (A.3.17) + (A.1.10), C1-477: I.A + (A.3.18) + (A.1.10), C1-478: I.A + (A.3.19) + (A.1.10),
C1-479: I.A + (A.3.20) + (A.1.10), C1-480: I.A + (A.3.21) + (A.1.10), C1-481: I.A + (A.3.22) +
(A.1.10), C1-482: I.A + (A.3.23) + (A.1.10), C1-483: I.A + (A.3.24) + (A.1.10), C1-484: I.A +
(A.3.25) + (A.1.10), C1-485: I.A + (A.3.26) + (A.1.10), C1-486: I.A + (A.3.27) + (A.1.10), C1-487:
I.A + (A.3.28) + (A.1.10), C1-488: I.A + (A.3.29) + (A.1.10), C1-489: I.A + (A.3.31) + (A.1.10),
C1-490: I.A + (A.3.32) + (A.1.10), C1-491: I.A + (A.3.33) + (A.1.10), C1-492: I.A + (A.3.34) +
(A.1.10), C1-493: I.A + (A.3.35) + (A.1.10), C1-494: I.A + (A.3.36) + (A.1.10), C1-495: I.A +
(A.3.37) + (A.1.10), C1-496: I.A + (A.3.38) + (A.1.10), C1-497: I.A + (A.3.39) + (A.1.10), C1-498:
I.A + (A.4.5) + (A.1.10), C1-499: I.A + (A.4.11) + (A.1.10), C1-500: I.A + (B.1.4) + (A.1.10), C1-
501: I.A + (B.1.5) + (A.1.10), C1-502: I.A + (B.1.8.) + (A.1.10), C1-503: I.A + (B.1.10) + (A.1.10),
C1-504: I.A + (B.1.11) + (A.1.10), C1-505: I.A + (B.1.12) + (A.1.10), C1-506: I.A + (B.1.13) +
(A.1.10), C1-507: I.A + (B.1.17) + (A.1.10), C1-508: I.A + (B.1.18) + (A.1.10), C1-509: I.A +
(B.1.21) + (A.1.10), C1-510: I.A + (B.1.22) + (A.1.10), C1-511: I.A + (B.1.23) + (A.1.10), C1-512:
I.A + (B.1.25) + (A.1.10), C1-513: I.A + (B.1.26) + (A.1.10), C1-514: I.A + (B.1.29) + (A.1.10),
C1-515: I.A + (B.1.33) + (A.1.10), C1-516: I.A + (B.1.34) + (A.1.10), C1-517: I.A + (B.1.37) +
(A.1.10), C1-518: I.A + (B.1.38) + (A.1.10), C1-519: I.A + (B.1.43) + (A.1.10), C1-520: I.A +
(B.1.46) + (A.1.10), C1-521: I.A + (B.2.4) + (A.1.10), C1-522: I.A + (B.2.5) + (A.1.10), C1-523:
I.A + (B.2.6) + (A.1.10), C1-524: I.A + (B.2.8) + (A.1.10), C1-525: I.A + (C.1.1) + (A.1.10), C1-
526: I.A + (C.1.2) + (A.1.10), C1-527: I.A + (C.1.4) + (A.1.10), C1-528: I.A + (C.1.5) + (A.1.10),
C1-529: I.A + (C.2.6) + (A.1.10), C1-530: I.A + (C.2.7) + (A.1.10), C1-531: I.A + (C.2.8) +
(A.1.10), C1-532: I.A + (D.1.1) + (A.1.10), C1-533: I.A + (D.1.2) + (A.1.10), C1-534: I.A + (D.1.5) +
(A.1.10), C1-535: I.A + (D.2.4) + (A.1.10), C1-536: I.A + (D.2.6) + (A.1.10), C1-537: I.A +
(D.2.7) + (A.1.10), C1-538: I.A + (E.1.1) + (A.1.10), C1-539: I.A + (E.1.3) + (A.1.10), C1-540: I.A +
(E.2.2) + (A.1.10), C1-541: I.A + (E.2.3) + (A.1.10), C1-542: I.A + (F.1.2) + (A.1.10), C1-543:
I.A + (F.1.4) + (A.1.10), C1-544: I.A + (F.1.5) + (A.1.10), C1-545: I.A + (G.3.1) + (A.1.10), C1-
546: I.A + (G.3.3) + (A.1.10), C1-547: I.A + (G.3.6) + (A.1.10), C1-548: I.A + (G.5.1) + (A.1.10),
C1-549: I.A + (G.5.2) + (A.1.10), C1-550: I.A + (G.5.3) + (A.1.10), C1-551: I.A + (G.5.4) +
(A.1.10), C1-552: I.A + (G.5.5) + (A.1.10), C1-553: I.A + (G.5.6) + (A.1.10), C1-554: I.A +
(G.5.7) + (A.1.10), C1-555: I.A + (G.5.8) + (A.1.10), C1-556: I.A + (G.5.9) + (A.1.10), C1-557:
I.A + (G.5.10) + (A.1.10), C1-558: I.A + (G.5.11) + (A.1.10), C1-559: I.A + (H.1.5) + (A.1.10),
C1-560: I.A + (H.1.7) + (A.1.10), C1-561: I.A + (H.2.2) + (A.1.10), C1-562: I.A + (H.2.3) +
(A.1.10), C1-563: I.A + (H.2.5) + (A.1.10), C1-564: I.A + (H.2.7) + (A.1.10), C1-565: I.A + (H.2.8) +
(A.1.10), C1-566: I.A + (H.3.2) + (A.1.10), C1-567: I.A + (H.3.4) + (A.1.10), C1-568: I.A +
(H.3.5) + (A.1.10), C1-569: I.A + (H.4.9) + (A.1.10), C1-570: I.A + (H.4.10) + (A.1.10), C1-571:
I.A + (I.2.2) + (A.1.10), C1-572: I.A + (I.2.5) + (A.1.10), C1-573: I.A + (J.1.2) + (A.1.10), C1-574:
I.A + (J.1.5) + (A.1.10), C1-575: I.A + (J.1.8) + (A.1.10), C1-576: I.A + (J.1.11) + (A.1.10), C1-
577: I.A + (J.1.12) + (A.1.10), C1-578: I.A + (K.1.3) + (A.1.10), C1-579: I.A + (K.1.29) + (A.1.10),
C1-580: I.A + (K.1.30) + (A.1.10), C1-581: I.A + (K.1.31) + (A.1.10), C1-582: I.A + (K.1.32) +
(A.1.10), C1-583: I.A + (K.1.41) + (A.1.10), C1-584: I.A + (K.1.42) + (A.1.10), C1-585: I.A +
(K.1.44) + (A.1.10), C1-586: I.A + (K.1.45) + (A.1.10), C1-587: I.A + (K.1.47) + (A.1.10), C1-588:
I.A + (K.1.49) + (A.1.10), C1-589: I.A + (K.1.53) + (A.1.10), C1-590: I.A + (K.1.54) + (A.1.10),
C1-591: I.A + (A.1.4) + (A.1.11), C1-592: I.A + (A.1.12) + (A.1.11), C1-593: I.A + (A.1.13) +
(A.1.11), C1-594: I.A + (A.1.14) + (A.1.11), C1-595: I.A + (A.1.17) + (A.1.11), C1-596: I.A +
(A.1.21) + (A.1.11), C1-597: I.A + (A.1.21a) + (A.1.11), C1-598: I.A + (A.1.24) + (A.1.11), C1-
599: I.A + (A.1.25) + (A.1.11), C1-600: I.A + (A.1.26) + (A.1.11), C1-601: I.A + (A.1.27) +
(A.1.11), C1-602: I.A + (A.1.30) + (A.1.11), C1-603: I.A + (A.1.31) + (A.1.11), C1-604: I.A +
(A.1.32) + (A.1.11), C1-605: I.A + (A.1.34) + (A.1.11), C1-606: I.A + (A.1.35) + (A.1.11), C1-607:
I.A + (A.2.1) + (A.1.11), C1-608: I.A + (A.2.3) + (A.1.11), C1-609: I.A + (A.2.4) + (A.1.11), C1-
610: I.A + (A.2.5) + (A.1.11), C1-611: I.A + (A.2.6) + (A.1.11), C1-612: I.A + (A.3.2) + (A.1.11),
C1-613: I.A + (A.3.3) + (A.1.11), C1-614: I.A + (A.3.4) + (A.1.11), C1-615: I.A + (A.3.7) +
(A.1.11), C1-616: I.A + (A.3.9) + (A.1.11), C1-617: I.A + (A.3.11) + (A.1.11), C1-618: I.A +
(A.3.12) + (A.1.11), C1-619: I.A + (A.3.15) + (A.1.11), C1-620: I.A + (A.3.16) + (A.1.11), C1-621:
I.A + (A.3.17) + (A.1.11), C1-622: I.A + (A.3.18) + (A.1.11), C1-623: I.A + (A.3.19) + (A.1.11),
C1-624: I.A + (A.3.20) + (A.1.11), C1-625: I.A + (A.3.21) + (A.1.11), C1-626: I.A + (A.3.22) +
(A.1.11), C1-627: I.A + (A.3.23) + (A.1.11), C1-628: I.A + (A.3.24) + (A.1.11), C1-629: I.A +
(A.3.25) + (A.1.11), C1-630: I.A + (A.3.26) + (A.1.11), C1-631: I.A + (A.3.27) + (A.1.11), C1-632:
I.A + (A.3.28) + (A.1.11), C1-633: I.A + (A.3.29) + (A.1.11), C1-634: I.A + (A.3.31) + (A.1.11),
C1-635: I.A + (A.3.32) + (A.1.11), C1-636: I.A + (A.3.33) + (A.1.11), C1-637: I.A + (A.3.34) +
(A.1.11), C1-638: I.A + (A.3.35) + (A.1.11), C1-639: I.A + (A.3.36) + (A.1.11), C1-640: I.A +
(A.3.37) + (A.1.11), C1-641: I.A + (A.3.38) + (A.1.11), C1-642: I.A + (A.3.39) + (A.1.11), C1-643:
I.A + (A.4.5) + (A.1.11), C1-644: I.A + (A.4.11) + (A.1.11), C1-645: I.A + (B.1.4) + (A.1.11), C1-
646: I.A + (B.1.5) + (A.1.11), C1-647: I.A + (B.1.8.) + (A.1.11), C1-648: I.A + (B.1.10) + (A.1.11),
C1-649: I.A + (B.1.11) + (A.1.11), C1-650: I.A + (B.1.12) + (A.1.11), C1-651: I.A + (B.1.13) +
(A.1.11), C1-652: I.A + (B.1.17) + (A.1.11), C1-653: I.A + (B.1.18) + (A.1.11), C1-654: I.A +
(B.1.21) + (A.1.11), C1-655: I.A + (B.1.22) + (A.1.11), C1-656: I.A + (B.1.23) + (A.1.11), C1-657:
I.A + (B.1.25) + (A.1.11), C1-658: I.A + (B.1.26) + (A.1.11), C1-659: I.A + (B.1.29) + (A.1.11),
C1-660: I.A + (B.1.33) + (A.1.11), C1-661: I.A + (B.1.34) + (A.1.11), C1-662: I.A + (B.1.37) +
(A.1.11), C1-663: I.A + (B.1.38) + (A.1.11), C1-664: I.A + (B.1.43) + (A.1.11), C1-665: I.A +
(B.1.46) + (A.1.11), C1-666: I.A + (B.2.4) + (A.1.11), C1-667: I.A + (B.2.5) + (A.1.11), C1-668:
I.A + (B.2.6) + (A.1.11), C1-669: I.A + (B.2.8) + (A.1.11), C1-670: I.A + (C.1.1) + (A.1.11), C1-
671: I.A + (C.1.2) + (A.1.11), C1-672: I.A + (C.1.4) + (A.1.11), C1-673: I.A + (C.1.5) + (A.1.11),
C1-674: I.A + (C.2.6) + (A.1.11), C1-675: I.A + (C.2.7) + (A.1.11), C1-676: I.A + (C.2.8) +
(A.1.11), C1-677: I.A + (D.1.1) + (A.1.11), C1-678: I.A + (D.1.2) + (A.1.11), C1-679: I.A + (D.1.5) +
(A.1.11), C1-680: I.A + (D.2.4) + (A.1.11), C1-681: I.A + (D.2.6) + (A.1.11), C1-682: I.A +
(D.2.7) + (A.1.11), C1-683: I.A + (E.1.1) + (A.1.11), C1-684: I.A + (E.1.3) + (A.1.11), C1-685: I.A +
(E.2.2) + (A.1.11), C1-686: I.A + (E.2.3) + (A.1.11), C1-687: I.A + (F.1.2) + (A.1.11), C1-688:
I.A + (F.1.4) + (A.1.11), C1-689: I.A + (F.1.5) + (A.1.11), C1-690: I.A + (G.3.1) + (A.1.11), C1-
691: I.A + (G.3.3) + (A.1.11), C1-692: I.A + (G.3.6) + (A.1.11), C1-693: I.A + (G.5.1) + (A.1.11),
C1-694: I.A + (G.5.2) + (A.1.11), C1-695: I.A + (G.5.3) + (A.1.11), C1-696: I.A + (G.5.4) +
(A.1.11), C1-697: I.A + (G.5.5) + (A.1.11), C1-698: I.A + (G.5.6) + (A.1.11), C1-699: I.A +
(G.5.7) + (A.1.11), C1-700: I.A + (G.5.8) + (A.1.11), C1-701: I.A + (G.5.9) + (A.1.11), C1-702:
I.A + (G.5.10) + (A.1.11), C1-703: I.A + (G.5.11) + (A.1.11), C1-704: I.A + (H.1.5) + (A.1.11),
C1-705: I.A + (H.1.7) + (A.1.11), C1-706: I.A + (H.2.2) + (A.1.11), C1-707: I.A + (H.2.3) +
(A.1.11), C1-708: I.A + (H.2.5) + (A.1.11), C1-709: I.A + (H.2.7) + (A.1.11), C1-710: I.A + (H.2.8) +
(A.1.11), C1-711: I.A + (H.3.2) + (A.1.11), C1-712: I.A + (H.3.4) + (A.1.11), C1-713: I.A +
(H.3.5) + (A.1.11), C1-714: I.A + (H.4.9) + (A.1.11), C1-715: I.A + (H.4.10) + (A.1.11), C1-716:
I.A + (I.2.2) + (A.1.11), C1-717: I.A + (I.2.5) + (A.1.11), C1-718: I.A + (J.1.2) + (A.1.11), C1-719:
I.A + (J.1.5) + (A.1.11), C1-720: I.A + (J.1.8) + (A.1.11), C1-721: I.A + (J.1.11) + (A.1.11), C1-
722: I.A + (J.1.12) + (A.1.11), C1-723: I.A + (K.1.3) + (A.1.11), C1-724: I.A + (K.1.29) + (A.1.11),
C1-725: I.A + (K.1.30) + (A.1.11), C1-726: I.A + (K.1.31) + (A.1.11), C1-727: I.A + (K.1.32) +
(A.1.11), C1-728: I.A + (K.1.41) + (A.1.11), C1-729: I.A + (K.1.42) + (A.1.11), C1-730: I.A +
(K.1.44) + (A.1.11), C1-731: I.A + (K.1.45) + (A.1.11), C1-732: I.A + (K.1.47) + (A.1.11), C1-733:
I.A + (K.1.49) + (A.1.11), C1-734: I.A + (K.1.53) + (A.1.11), C1-735: I.A + (K.1.54) + (A.1.11),
C1-736: I.A + (A.1.4) + (A.1.13), C1-737: I.A + (A.1.12) + (A.1.13), C1-738: I.A + (A.1.14) +
(A.1.13), C1-739: I.A + (A.1.17) + (A.1.13), C1-740: I.A + (A.1.21) + (A.1.13), C1-741: I.A +
(A.1.21a) + (A.1.13), C1-742: I.A + (A.1.24) + (A.1.13), C1-743: I.A + (A.1.25) + (A.1.13), C1-
744: I.A + (A.1.26) + (A.1.13), C1-745: I.A + (A.1.27) + (A.1.13), C1-746: I.A + (A.1.30) +
(A.1.13), C1-747: I.A + (A.1.31) + (A.1.13), C1-748: I.A + (A.1.32) + (A.1.13), C1-749: I.A +
(A.1.34) + (A.1.13), C1-750: I.A + (A.1.35) + (A.1.13), C1-751: I.A + (A.2.1) + (A.1.13), C1-752:
I.A + (A.2.3) + (A.1.13), C1-753: I.A + (A.2.4) + (A.1.13), C1-754: I.A + (A.2.5) + (A.1.13), C1-
755: I.A + (A.2.6) + (A.1.13), C1-756: I.A + (A.3.2) + (A.1.13), C1-757: I.A + (A.3.3) + (A.1.13),
C1-758: I.A + (A.3.4) + (A.1.13), C1-759: I.A + (A.3.7) + (A.1.13), C1-760: I.A + (A.3.9) +
(A.1.13), C1-761: I.A + (A.3.11) + (A.1.13), C1-762: I.A + (A.3.12) + (A.1.13), C1-763: I.A +
(A.3.15) + (A.1.13), C1-764: I.A + (A.3.16) + (A.1.13), C1-765: I.A + (A.3.17) + (A.1.13), C1-766:
I.A + (A.3.18) + (A.1.13), C1-767: I.A + (A.3.19) + (A.1.13), C1-768: I.A + (A.3.20) + (A.1.13),
C1-769: I.A + (A.3.21) + (A.1.13), C1-770: I.A + (A.3.22) + (A.1.13), C1-771: I.A + (A.3.23) +
(A.1.13), C1-772: I.A + (A.3.24) + (A.1.13), C1-773: I.A + (A.3.25) + (A.1.13), C1-774: I.A +
(A.3.26) + (A.1.13), C1-775: I.A + (A.3.27) + (A.1.13), C1-776: I.A + (A.3.28) + (A.1.13), C1-777:
I.A + (A.3.29) + (A.1.13), C1-778: I.A + (A.3.31) + (A.1.13), C1-779: I.A + (A.3.32) + (A.1.13),
C1-780: I.A + (A.3.33) + (A.1.13), C1-781: I.A + (A.3.34) + (A.1.13), C1-782: I.A + (A.3.35) +
(A.1.13), C1-783: I.A + (A.3.36) + (A.1.13), C1-784: I.A + (A.3.37) + (A.1.13), C1-785: I.A +
(A.3.38) + (A.1.13), C1-786: I.A + (A.3.39) + (A.1.13), C1-787: I.A + (A.4.5) + (A.1.13), C1-788:
I.A + (A.4.11) + (A.1.13), C1-789: I.A + (B.1.4) + (A.1.13), C1-790: I.A + (B.1.5) + (A.1.13), C1-
791: I.A + (B.1.8.) + (A.1.13), C1-792: I.A + (B.1.10) + (A.1.13), C1-793: I.A + (B.1.11) +
(A.1.13), C1-794: I.A + (B.1.12) + (A.1.13), C1-795: I.A + (B.1.13) + (A.1.13), C1-796: I.A +
(B.1.17) + (A.1.13), C1-797: I.A + (B.1.18) + (A.1.13), C1-798: I.A + (B.1.21) + (A.1.13), C1-799:
I.A + (B.1.22) + (A.1.13), C1-800: I.A + (B.1.23) + (A.1.13), C1-801: I.A + (B.1.25) + (A.1.13),
C1-802: I.A + (B.1.26) + (A.1.13), C1-803: I.A + (B.1.29) + (A.1.13), C1-804: I.A + (B.1.33) +
(A.1.13), C1-805: I.A + (B.1.34) + (A.1.13), C1-806: I.A + (B.1.37) + (A.1.13), C1-807: I.A +
(B.1.38) + (A.1.13), C1-808: I.A + (B.1.43) + (A.1.13), C1-809: I.A + (B.1.46) + (A.1.13), C1-810:
I.A + (B.2.4) + (A.1.13), C1-811: I.A + (B.2.5) + (A.1.13), C1-812: I.A + (B.2.6) + (A.1.13), C1-
813: I.A + (B.2.8) + (A.1.13), C1-814: I.A + (C.1.1) + (A.1.13), C1-815: I.A + (C.1.2) + (A.1.13),
C1-816: I.A + (C.1.4) + (A.1.13), C1-817: I.A + (C.1.5) + (A.1.13), C1-818: I.A + (C.2.6) +
(A.1.13), C1-819: I.A + (C.2.7) + (A.1.13), C1-820: I.A + (C.2.8) + (A.1.13), C1-821: I.A + (D.1.1) +
(A.1.13), C1-822: I.A + (D.1.2) + (A.1.13), C1-823: I.A + (D.1.5) + (A.1.13), C1-824: I.A +
(D.2.4) + (A.1.13), C1-825: I.A + (D.2.6) + (A.1.13), C1-826: I.A + (D.2.7) + (A.1.13), C1-827: I.A +
(E.1.1) + (A.1.13), C1-828: I.A + (E.1.3) + (A.1.13), C1-829: I.A + (E.2.2) + (A.1.13), C1-830:
I.A + (E.2.3) + (A.1.13), C1-831: I.A + (F.1.2) + (A.1.13), C1-832: I.A + (F.1.4) + (A.1.13), C1-
833: I.A + (F.1.5) + (A.1.13), C1-834: I.A + (G.3.1) + (A.1.13), C1-835: I.A + (G.3.3) + (A.1.13),
C1-836: I.A + (G.3.6) + (A.1.13), C1-837: I.A + (G.5.1) + (A.1.13), C1-838: I.A + (G.5.2) +
(A.1.13), C1-839: I.A + (G.5.3) + (A.1.13), C1-840: I.A + (G.5.4) + (A.1.13), C1-841: I.A +
(G.5.5) + (A.1.13), C1-842: I.A + (G.5.6) + (A.1.13), C1-843: I.A + (G.5.7) + (A.1.13), C1-844:
I.A + (G.5.8) + (A.1.13), C1-845: I.A + (G.5.9) + (A.1.13), C1-846: I.A + (G.5.10) + (A.1.13), C1-
847: I.A + (G.5.11) + (A.1.13), C1-848: I.A + (H.1.5) + (A.1.13), C1-849: I.A + (H.1.7) + (A.1.13),
C1-850: I.A + (H.2.2) + (A.1.13), C1-851: I.A + (H.2.3) + (A.1.13), C1-852: I.A + (H.2.5) +
(A.1.13), C1-853: I.A + (H.2.7) + (A.1.13), C1-854: I.A + (H.2.8) + (A.1.13), C1-855: I.A + (H.3.2) +
(A.1.13), C1-856: I.A + (H.3.4) + (A.1.13), C1-857: I.A + (H.3.5) + (A.1.13), C1-858: I.A +
(H.4.9) + (A.1.13), C1-859: I.A + (H.4.10) + (A.1.13), C1-860: I.A + (I.2.2) + (A.1.13), C1-861: I.A +
(I.2.5) + (A.1.13), C1-862: I.A + (J.1.2) + (A.1.13), C1-863: I.A + (J.1.5) + (A.1.13), C1-864: I.A +
(J.1.8) + (A.1.13), C1-865: I.A + (J.1.11) + (A.1.13), C1-866: I.A + (J.1.12) + (A.1.13), C1-867:
I.A + (K.1.3) + (A.1.13), C1-868: I.A + (K.1.29) + (A.1.13), C1-869: I.A + (K.1.30) + (A.1.13), C1-
870: I.A + (K.1.31) + (A.1.13), C1-871: I.A + (K.1.32) + (A.1.13), C1-872: I.A + (K.1.41) +
(A.1.13), C1-873: I.A + (K.1.42) + (A.1.13), C1-874: I.A + (K.1.44) + (A.1.13), C1-875: I.A +
(K.1.45) + (A.1.13), C1-876: I.A + (K.1.47) + (A.1.13), C1-877: I.A + (K.1.49) + (A.1.13), C1-878:
I.A + (K.1.53) + (A.1.13), C1-879: I.A + (K.1.54) + (A.1.13), C1-880: I.A + (A.1.4) + (A.1.14), C1-
881: I.A + (A.1.12) + (A.1.14), C1-882: I.A + (A.1.17) + (A.1.14), C1-883: I.A + (A.1.21) +
(A.1.14), C1-884: I.A + (A.1,21a) + (A.1.14), C1-885: I.A + (A.1.24) + (A.1.14), C1-886: I.A +
(A.1.25) + (A.1.14), C1-887: I.A + (A.1.26) + (A.1.14), C1-888: I.A + (A.1.27) + (A.1.14), C1-889:
I.A + (A.1.30) + (A.1.14), C1-890: I.A + (A.1.31) + (A.1.14), C1-891: I.A + (A.1.32) + (A.1.14),
C1-892: I.A + (A.1.34) + (A.1.14), C1-893: I.A + (A.1.35) + (A.1.14), C1-894: I.A + (A.2.1) +
(A.1.14), C1-895: I.A + (A.2.3) + (A.1.14), C1-896: I.A + (A.2.4) + (A.1.14), C1-897: I.A + (A.2.5) +
(A.1.14), C1-898: I.A + (A.2.6) + (A.1.14), C1-899: I.A + (A.3.2) + (A.1.14), C1-900: I.A +
(A.3.3) + (A.1.14), C1-901: I.A + (A.3.4) + (A.1.14), C1-902: I.A + (A.3.7) + (A.1.14), C1-903: I.A +
(A.3.9) + (A.1.14), C1-904: I.A + (A.3.11) + (A.1.14), C1-905: I.A + (A.3.12) + (A.1.14), C1-
906: I.A + (A.3.15) + (A.1.14), C1-907: I.A + (A.3.16) + (A.1.14), C1-908: I.A + (A.3.17) +
(A.1.14), C1-909: I.A + (A.3.18) + (A.1.14), C1-910: I.A + (A.3.19) + (A.1.14), C1-911: I.A +
(A.3.20) + (A.1.14), C1-912: I.A + (A.3.21) + (A.1.14), C1-913: I.A + (A.3.22) + (A.1.14), C1-914:
I.A + (A.3.23) + (A.1.14), C1-915: I.A + (A.3.24) + (A.1.14), C1-916: I.A + (A.3.25) + (A.1.14),
C1-917: I.A + (A.3.26) + (A.1.14), C1-918: I.A + (A.3.27) + (A.1.14), C1-919: I.A + (A.3.28) +
(A.1.14), C1-920: I.A + (A.3.29) + (A.1.14), C1-921: I.A + (A.3.31) + (A.1.14), C1-922: I.A +
(A.3.32) + (A.1.14), C1-923: I.A + (A.3.33) + (A.1.14), C1-924: I.A + (A.3.34) + (A.1.14), C1-925:
I.A + (A.3.35) + (A.1.14), C1-926: I.A + (A.3.36) + (A.1.14), C1-927: I.A + (A.3.37) + (A.1.14),
C1-928: I.A + (A.3.38) + (A.1.14), C1-929: I.A + (A.3.39) + (A.1.14), C1-930: I.A + (A.4.5) +
(A.1.14), C1-931: I.A + (A.4.11) + (A.1.14), C1-932: I.A + (B.1.4) + (A.1.14), C1-933: I.A +
(B.1.5) + (A.1.14), C1-934: I.A + (B.1.8.) + (A.1.14), C1-935: I.A + (B.1.10) + (A.1.14), C1-936:
I.A + (B.1.11) + (A.1.14), C1-937: I.A + (B.1.12) + (A.1.14), C1-938: I.A + (B.1.13) + (A.1.14),
C1-939: I.A + (B.1.17) + (A.1.14), C1-940: I.A + (B.1.18) + (A.1.14), C1-941: I.A + (B.1.21) +
(A.1.14), C1-942: I.A + (B.1.22) + (A.1.14), C1-943: I.A + (B.1.23) + (A.1.14), C1-944: I.A +
(B.1.25) + (A.1.14), C1-945: I.A + (B.1.26) + (A.1.14), C1-946: I.A + (B.1.29) + (A.1.14), C1-947:
I.A + (B.1.33) + (A.1.14), C1-948: I.A + (B.1.34) + (A.1.14), C1-949: I.A + (B.1.37) + (A.1.14),
C1-950: I.A + (B.1.38) + (A.1.14), C1-951: I.A + (B.1.43) + (A.1.14), C1-952: I.A + (B.1.46) +
(A.1.14), C1-953: I.A + (B.2.4) + (A.1.14), C1-954: I.A + (B.2.5) + (A.1.14), C1-955: I.A + (B.2.6) +
(A.1.14), C1-956: I.A + (B.2.8) + (A.1.14), C1-957: I.A + (C.1.1) + (A.1.14), C1-958: I.A +
(C.1.2) + (A.1.14), C1-959: I.A + (C.1.4) + (A.1.14), C1-960: I.A + (C.1.5) + (A.1.14), C1-961: I.A +
(C.2.6) + (A.1.14), C1-962: I.A + (C.2.7) + (A.1.14), C1-963: I.A + (C.2.8) + (A.1.14), C1-964:
I.A + (D.1.1) + (A.1.14), C1-965: I.A + (D.1.2) + (A.1.14), C1-966: I.A + (D.1.5) + (A.1.14), C1-
967: I.A + (D.2.4) + (A.1.14), C1-968: I.A + (D.2.6) + (A.1.14), C1-969: I.A + (D.2.7) + (A.1.14),
C1-970: I.A + (E.1.1) + (A.1.14), C1-971: I.A + (E.1.3) + (A.1.14), C1-972: I.A + (E.2.2) +
(A.1.14), C1-973: I.A + (E.2.3) + (A.1.14), C1-974: I.A + (F.1.2) + (A.1.14), C1-975: I.A + (F.1.4) +
(A.1.14), C1-976: I.A + (F.1.5) + (A.1.14), C1-977: I.A + (G.3.1) + (A.1.14), C1-978: I.A +
(G.3.3) + (A.1.14), C1-979: I.A + (G.3.6) + (A.1.14), C1-980: I.A + (G.5.1) + (A.1.14), C1-981:
I.A + (G.5.2) + (A.1.14), C1-982: I.A + (G.5.3) + (A.1.14), C1-983: I.A + (G.5.4) + (A.1.14), C1-
984: I.A + (G.5.5) + (A.1.14), C1-985: I.A + (G.5.6) + (A.1.14), C1-986: I.A + (G.5.7) + (A.1.14),
C1-987: I.A + (G.5.8) + (A.1.14), C1-988: I.A + (G.5.9) + (A.1.14), C1-989: I.A + (G.5.10) +
(A.1.14), C1-990: I.A + (G.5.11) + (A.1.14), C1-991: I.A + (H.1.5) + (A.1.14), C1-992: I.A +
(H.1.7) + (A.1.14), C1-993: I.A + (H.2.2) + (A.1.14), C1-994: I.A + (H.2.3) + (A.1.14), C1-995: I.A +
(H.2.5) + (A.1.14), C1-996: I.A + (H.2.7) + (A.1.14), C1-997: I.A + (H.2.8) + (A.1.14), C1-998:
I.A + (H.3.2) + (A.1.14), C1-999: I.A + (H.3.4) + (A.1.14), C1-1000: I.A + (H.3.5) + (A.1.14), C1-
1001: I.A + (H.4.9) + (A.1.14), C1-1002: I.A + (H.4.10) + (A.1.14), C1-1003: I.A + (I.2.2) +
(A.1.14), C1-1004: I.A + (I.2.5) + (A.1.14), C1-1005: I.A + (J.1.2) + (A.1.14), C1-1006: I.A +
(J.1.5) + (A.1.14), C1-1007: I.A + (J.1.8) + (A.1.14), C1-1008: I.A + (J.1.11) + (A.1.14), C1-1009:
I.A + (J.1.12) + (A.1.14), C1-1010: I.A + (K.1.3) + (A.1.14), C1-1011: I.A + (K.1.29) + (A.1.14),
C1-1012: I.A + (K.1.30) + (A.1.14), C1-1013: I.A + (K.1.31) + (A.1.14), C1-1014: I.A + (K.1.32) +
(A.1.14), C1-1015: I.A + (K.1.41) + (A.1.14), C1-1016: I.A + (K.1.42) + (A.1.14), C1-1017: I.A +
(K.1.44) + (A.1.14), C1-1018: I.A + (K.1.45) + (A.1.14), C1-1019: I.A + (K.1.47) + (A.1.14), C1-
1020: I.A + (K.1.49) + (A.1.14), C1-1021: I.A + (K.1.53) + (A.1.14), C1-1022: I.A + (K.1.54) +
(A.1.14), C1-1023: I.A + (A.1.4) + (A.1.17), C1-1024: I.A + (A.1.12) + (A.1.17), C1-1025: I.A +
(A.1.21) + (A.1.17), C1-1026: I.A + (A.1,21a) + (A.1.17), C1-1027: I.A + (A.1.24) + (A.1.17), C1-
1028: I.A + (A.1.25) + (A.1.17), C1-1029: I.A + (A.1.26) + (A.1.17), C1-1030: I.A + (A.1.27) +
(A.1.17), C1-1031: I.A + (A.1.30) + (A.1.17), C1-1032: I.A + (A.1.31) + (A.1.17), C1-1033: I.A +
(A.1.32) + (A.1.17), C1-1034: I.A + (A.1.34) + (A.1.17), C1-1035: I.A + (A.1.35) + (A.1.17), C1-
1036: I.A + (A.2.1) + (A.1.17), C1-1037: I.A + (A.2.3) + (A.1.17), C1-1038: I.A + (A.2.4) +
(A.1.17), C1-1039: I.A + (A.2.5) + (A.1.17), C1-1040: I.A + (A.2.6) + (A.1.17), C1-1041: I.A +
(A.3.2) + (A.1.17), C1-1042: I.A + (A.3.3) + (A.1.17), C1-1043: I.A + (A.3.4) + (A.1.17), C1-1044:
I.A + (A.3.7) + (A.1.17), C1-1045: I.A + (A.3.9) + (A.1.17), C1-1046: I.A + (A.3.11) + (A.1.17),
C1-1047: I.A + (A.3.12) + (A.1.17), C1-1048: I.A + (A.3.15) + (A.1.17), C1-1049: I.A + (A.3.16) +
(A.1.17), C1-1050: I.A + (A.3.17) + (A.1.17), C1-1051:1.A + (A.3.18) + (A.1.17), C1-1052: I.A +
(A.3.19) + (A.1.17), C1-1053: I.A + (A.3.20) + (A.1.17), C1-1054: I.A + (A.3.21) + (A.1.17), C1-
1055: I.A + (A.3.22) + (A.1.17), C1-1056: I.A + (A.3.23) + (A.1.17), C1-1057: I.A + (A.3.24) +
(A.1.17), C1-1058: I.A + (A.3.25) + (A.1.17), C1-1059: I.A + (A.3.26) + (A.1.17), C1-1060: I.A +
(A.3.27) + (A.1.17), C1-1061: I.A + (A.3.28) + (A.1.17), C1-1062: I.A + (A.3.29) + (A.1.17), C1-
1063: I.A + (A.3.31) + (A.1.17), C1-1064: I.A + (A.3.32) + (A.1.17), C1-1065: I.A + (A.3.33) +
(A.1.17), C1-1066: I.A + (A.3.34) + (A.1.17), C1-1067: I.A + (A.3.35) + (A.1.17), C1-1068: I.A +
(A.3.36) + (A.1.17), C1-1069: I.A + (A.3.37) + (A.1.17), C1-1070: I.A + (A.3.38) + (A.1.17), C1-
1071: I.A + (A.3.39) + (A.1.17), C1-1072: I.A + (A.4.5) + (A.1.17), C1-1073: I.A + (A.4.11) +
(A.1.17), C1-1074: I.A + (B.1.4) + (A.1.17), C1-1075: I.A + (B.1.5) + (A.1.17), C1-1076: I.A +
(B.1.8.) + (A.1.17), C1-1077: I.A + (B.1.10) + (A.1.17), C1-1078: I.A + (B.1.11) + (A.1.17), C1-
1079: I.A + (B.1.12) + (A.1.17), C1-1080: I.A + (B.1.13) + (A.1.17), C1-1081: I.A + (B.1.17) +
(A.1.17), C1-1082: I.A + (B.1.18) + (A.1.17), C1-1083: I.A + (B.1.21) + (A.1.17), C1-1084: I.A +
(B.1.22) + (A.1.17), C1-1085: I.A + (B.1.23) + (A.1.17), C1-1086: I.A + (B.1.25) + (A.1.17), C1-
1087: I.A + (B.1.26) + (A.1.17), C1-1088: I.A + (B.1.29) + (A.1.17), C1-1089: I.A + (B.1.33) +
(A.1.17), C1-1090: I.A + (B.1.34) + (A.1.17), C1-1091: I.A + (B.1.37) + (A.1.17), C1-1092: I.A +
(B.1.38) + (A.1.17), C1-1093: I.A + (B.1.43) + (A.1.17), C1-1094: I.A + (B.1.46) + (A.1.17), C1-
1095: I.A + (B.2.4) + (A.1.17), C1-1096: I.A + (B.2.5) + (A.1.17), C1-1097: I.A + (B.2.6) +
(A.1.17), C1-1098: I.A + (B.2.8) + (A.1.17), C1-1099: I.A + (C.1.1) + (A.1.17), C1-1100: I.A +
(C.1.2) + (A.1.17), C1-1101: I.A + (C.1.4) + (A.1.17), C1-1102: I.A + (C.1.5) + (A.1.17), C1-
1103: I.A + (C.2.6) + (A.1.17), C1-1104: I.A + (C.2.7) + (A.1.17), C1-1105: I.A + (C.2.8) +
(A.1.17), C1-1106: I.A + (D.1.1) + (A.1.17), C1-1107: I.A + (D.1.2) + (A.1.17), C1-1108: I.A +
(D.1.5) + (A.1.17), C1-1109: I.A + (D.2.4) + (A.1.17), C1-1110: I.A + (D.2.6) + (A.1.17), C1-
1111: I.A + (D.2.7) + (A.1.17), C1-1112: I.A + (E.1.1) + (A.1.17), C1-1113: I.A + (E.1.3) +
(A.1.17), C1-1114: I.A + (E.2.2) + (A.1.17), C1-1115: I.A + (E.2.3) + (A.1.17), C1-1116: I.A +
(F.1.2) + (A.1.17), C1-1117: I.A + (F.1.4) + (A.1.17), C1-1118: I.A + (F.1.5) + (A.1.17), C1-1119:
I.A + (G.3.1) + (A.1.17), C1-1120: I.A + (G.3.3) + (A.1.17), C1-1121: I.A + (G.3.6) + (A.1.17),
C1-1122: I.A + (G.5.1) + (A.1.17), C1-1123: I.A + (G.5.2) + (A.1.17), C1-1124: I.A + (G.5.3) +
(A.1.17), C1-1125: I.A + (G.5.4) + (A.1.17), C1-1126: I.A + (G.5.5) + (A.1.17), C1-1127: I.A +
(G.5.6) + (A.1.17), C1-1128: I.A + (G.5.7) + (A.1.17), C1-1129: I.A + (G.5.8) + (A.1.17), C1-
1130: I.A + (G.5.9) + (A.1.17), C1-1131: I.A + (G.5.10) + (A.1.17), C1-1132: I.A + (G.5.11) +
(A.1.17), C1-1133: I.A + (H.1.5) + (A.1.17), C1-1134: I.A + (H.1.7) + (A.1.17), C1-1135: I.A +
(H.2.2) + (A.1.17), C1-1136: I.A + (H.2.3) + (A.1.17), C1-1137: I.A + (H.2.5) + (A.1.17), C1-
1138: I.A + (H.2.7) + (A.1.17), C1-1139: I.A + (H.2.8) + (A.1.17), C1-1140: I.A + (H.3.2) +
(A.1.17), C1-1141: I.A + (H.3.4) + (A.1.17), C1-1142: I.A + (H.3.5) + (A.1.17), C1-1143: I.A +
(H.4.9) + (A.1.17), C1-1144: I.A + (H.4.10) + (A.1.17), C1-1145: I.A + (I.2.2) + (A.1.17), C1-
1146: I.A + (I.2.5) + (A.1.17), C1-1147: I.A + (J.1.2) + (A.1.17), C1-1148: I.A + (J.1.5) + (A.1.17),
C1-1149: I.A + (J.1.8) + (A.1.17), C1-1150: I.A + (J.1.11) + (A.1.17), C1-1151: I.A + (J.1.12) +
(A.1.17), C1-1152: I.A + (K.1.3) + (A.1.17), C1-1153: I.A + (K.1.29) + (A.1.17), C1-1154: I.A +
(K.1.30) + (A.1.17), C1-1155: I.A + (K.1.31) + (A.1.17), C1-1156: I.A + (K.1.32) + (A.1.17), C1-
1157: I.A + (K.1.41) + (A.1.17), C1-1158: I.A + (K.1.42) + (A.1.17), C1-1159: I.A + (K.1.44) +
(A.1.17), C1-1160: I.A + (K.1.45) + (A.1.17), C1-1161: I.A + (K.1.47) + (A.1.17), C1-1162: I.A +
(K.1.49) + (A.1.17), C1-1163: I.A + (K.1.53) + (A.1.17), C1-1164: I.A + (K.1.54) + (A.1.17), C1-
1165: I.A + (A.1.4) + (A.1.25), C1-1166: I.A + (A.1.12) + (A.1.25), C1-1167: I.A + (A.1.21) +
(A.1.25), C1-1168: I.A + (A.1,21a) + (A.1.25), C1-1169: I.A + (A.1.24) + (A.1.25), C1-1170: I.A +
(A.1.26) + (A.1.25), C1-1171: I.A + (A.1.27) + (A.1.25), C1-1172: I.A + (A.1.30) + (A.1.25), C1-
1173: I.A + (A.1.31) + (A.1.25), C1-1174: I.A + (A.1.32) + (A.1.25), C1-1175: I.A + (A.1.34) +
(A.1.25), C1-1176: I.A + (A.1.35) + (A.1.25), C1-1177: I.A + (A.2.1) + (A.1.25), C1-1178: I.A +
(A.2.3) + (A.1.25), C1-1179: I.A + (A.2.4) + (A.1.25), C1-1180: I.A + (A.2.5) + (A.1.25), C1-1181:
I.A + (A.2.6) + (A.1.25), C1-1182: I.A + (A.3.2) + (A.1.25), C1-1183: I.A + (A.3.3) + (A.1.25), C1-
1184: I.A + (A.3.4) + (A.1.25), C1-1185: I.A + (A.3.7) + (A.1.25), C1-1186: I.A + (A.3.9) +
(A.1.25), C1-1187: I.A + (A.3.11) + (A.1.25), C1-1188: I.A + (A.3.12) + (A.1.25), C1-1189: I.A +
(A.3.15) + (A.1.25), C1-1190: I.A + (A.3.16) + (A.1.25), C1-1191: I.A + (A.3.17) + (A.1.25), C1-
1192: I.A + (A.3.18) + (A.1.25), C1-1193: I.A + (A.3.19) + (A.1.25), C1-1194: I.A + (A.3.20) +
(A.1.25), C1-1195: I.A + (A.3.21) + (A.1.25), C1-1196: I.A + (A.3.22) + (A.1.25), C1-1197: I.A +
(A.3.23) + (A.1.25), C1-1198: I.A + (A.3.24) + (A.1.25), C1-1199: I.A + (A.3.25) + (A.1.25), C1-
1200: I.A + (A.3.26) + (A.1.25), C1-1201: I.A + (A.3.27) + (A.1.25), C1-1202: I.A + (A.3.28) +
(A.1.25), C1-1203: I.A + (A.3.29) + (A.1.25), C1-1204: I.A + (A.3.31) + (A.1.25), C1-1205: I.A +
(A.3.32) + (A.1.25), C1-1206: I.A + (A.3.33) + (A.1.25), C1-1207: I.A + (A.3.34) + (A.1.25), C1-
1208: I.A + (A.3.35) + (A.1.25), C1-1209: I.A + (A.3.36) + (A.1.25), C1-1210: I.A + (A.3.37) +
(A.1.25), C1-1211: I.A + (A.3.38) + (A.1.25), C1-1212: I.A + (A.3.39) + (A.1.25), C1-1213: I.A +
(A.4.5) + (A.1.25), C1-1214: I.A + (A.4.11) + (A.1.25), C1-1215: I.A + (B.1.4) + (A.1.25), C1-
1216: I.A + (B.1.5) + (A.1.25), C1-1217: I.A + (B.1.8.) + (A.1.25), C1-1218: I.A + (B.1.10) +
(A.1.25), C1-1219: I.A + (B.1.11) + (A.1.25), C1-1220: I.A + (B.1.12) + (A.1.25), C1-1221: I.A +
(B.1.13) + (A.1.25), C1-1222: I.A + (B.1.17) + (A.1.25), C1-1223: I.A + (B.1.18) + (A.1.25), C1-
1224: I.A + (B.1.21) + (A.1.25), C1-1225: I.A + (B.1.22) + (A.1.25), C1-1226: I.A + (B.1.23) +
(A.1.25), C1-1227: I.A + (B.1.25) + (A.1.25), C1-1228: I.A + (B.1.26) + (A.1.25), C1-1229: I.A +
(B.1.29) + (A.1.25), C1-1230: I.A + (B.1.33) + (A.1.25), C1-1231: I.A + (B.1.34) + (A.1.25), C1-
1232: I.A + (B.1.37) + (A.1.25), C1-1233: I.A + (B.1.38) + (A.1.25), C1-1234: I.A + (B.1.43) +
(A.1.25), C1-1235: I.A + (B.1.46) + (A.1.25), C1-1236: I.A + (B.2.4) + (A.1.25), C1-1237: I.A +
(B.2.5) + (A.1.25), C1-1238: I.A + (B.2.6) + (A.1.25), C1-1239: I.A + (B.2.8) + (A.1.25), C1-1240:
I.A + (C.1.1) + (A.1.25), C1-1241: I.A + (C.1.2) + (A.1.25), C1-1242: I.A + (C.1.4) + (A.1.25), C1-
1243: I.A + (C.1.5) + (A.1.25), C1-1244: I.A + (C.2.6) + (A.1.25), C1-1245: I.A + (C.2.7) +
(A.1.25), C1-1246: I.A + (C.2.8) + (A.1.25), C1-1247: I.A + (D.1.1) + (A.1.25), C1-1248: I.A +
(D.1.2) + (A.1.25), C1-1249: I.A + (D.1.5) + (A.1.25), C1-1250: I.A + (D.2.4) + (A.1.25), C1-
1251: I.A + (D.2.6) + (A.1.25), C1-1252: I.A + (D.2.7) + (A.1.25), C1-1253: I.A + (E.1.1) +
(A.1.25), C1-1254: I.A + (E.1.3) + (A.1.25), C1-1255: I.A + (E.2.2) + (A.1.25), C1-1256: I.A +
(E.2.3) + (A.1.25), C1-1257: I.A + (F.1.2) + (A.1.25), C1-1258: I.A + (F.1.4) + (A.1.25), C1-1259:
I.A + (F.1.5) + (A.1.25), C1-1260: I.A + (G.3.1) + (A.1.25), C1-1261: I.A + (G.3.3) + (A.1.25), C1-
1262: I.A + (G.3.6) + (A.1.25), C1-1263: I.A + (G.5.1) + (A.1.25), C1-1264: I.A + (G.5.2) +
(A.1.25), C1-1265: I.A + (G.5.3) + (A.1.25), C1-1266: I.A + (G.5.4) + (A.1.25), C1-1267: I.A +
(G.5.5) + (A.1.25), C1-1268: I.A + (G.5.6) + (A.1.25), C1-1269: I.A + (G.5.7) + (A.1.25), C1-
1270: I.A + (G.5.8) + (A.1.25), C1-1271: I.A + (G.5.9) + (A.1.25), C1-1272: I.A + (G.5.10) +
(A.1.25), C1-1273: I.A + (G.5.11) + (A.1.25), C1-1274: I.A + (H.1.5) + (A.1.25), C1-1275: I.A +
(H.1.7) + (A.1.25), C1-1276: I.A + (H.2.2) + (A.1.25), C1-1277: I.A + (H.2.3) + (A.1.25), C1-
1278: I.A + (H.2.5) + (A.1.25), C1-1279: I.A + (H.2.7) + (A.1.25), C1-1280: I.A + (H.2.8) +
(A.1.25), C1-1281: I.A + (H.3.2) + (A.1.25), C1-1282: I.A + (H.3.4) + (A.1.25), C1-1283: I.A +
(H.3.5) + (A.1.25), C1-1284: I.A + (H.4.9) + (A.1.25), C1-1285: I.A + (H.4.10) + (A.1.25), C1-
1286: I.A + (I.2.2) + (A.1.25), C1-1287: I.A + (I.2.5) + (A.1.25), C1-1288: I.A + (J. 1.2) + (A.1.25),
C1-1289: I.A + (J.1.5) + (A.1.25), C1-1290: I.A + (J.1.8) + (A.1.25), C1-1291: I.A + (J.1.11) +
(A.1.25), C1-1292: I.A + (J.1.12) + (A.1.25), C1-1293: I.A + (K.1.3) + (A.1.25), C1-1294: I.A +
(K.1.29) + (A.1.25), C1-1295: I.A + (K.1.30) + (A.1.25), C1-1296: I.A + (K.1.31) + (A.1.25), C1-
1297: I.A + (K.1.32) + (A.1.25), C1-1298: I.A + (K.1.41) + (A.1.25), C1-1299: I.A + (K.1.42) +
(A.1.25), C1-1300: I.A + (K.1.44) + (A.1.25), C1-1301: I.A + (K.1.45) + (A.1.25), C1-1302: I.A +
(K.1.47) + (A.1.25), C1-1303: I.A + (K.1.49) + (A.1.25), C1-1304: I.A + (K.1.53) + (A.1.25), C1-
1305: I.A + (K.1.54) + (A.1.25), C1-1306: I.A + (A.1.4) + (A.1.34), C1-1307: I.A + (A.1.12) +
(A.1.34), C1-1308: I.A + (A.1.21) + (A.1.34), C1-1309: I.A + (A.1,21a) + (A.1.34), C1-1310: I.A +
(A.1.24) + (A.1.34), C1-1311: I.A + (A.1.26) + (A.1.34), C1-1312: I.A + (A.1.27) + (A.1.34), C1-
1313: I.A + (A.1.30) + (A.1.34), C1-1314: I.A + (A.1.31) + (A.1.34), C1-1315: I.A + (A.1.32) +
(A.1.34), C1-1316: I.A + (A.1.35) + (A.1.34), C1-1317: I.A + (A.2.1) + (A.1.34), C1-1318: I.A +
(A.2.3) + (A.1.34), C1-1319: I.A + (A.2.4) + (A.1.34), C1-1320: I.A + (A.2.5) + (A.1.34), C1-1321:
I.A + (A.2.6) + (A.1.34), C1-1322: I.A + (A.3.2) + (A.1.34), C1-1323: I.A + (A.3.3) + (A.1.34), C1-
1324: I.A + (A.3.4) + (A.1.34), C1-1325: I.A + (A.3.7) + (A.1.34), C1-1326: I.A + (A.3.9) +
(A.1.34), C1-1327: I.A + (A.3.11) + (A.1.34), C1-1328: I.A + (A.3.12) + (A.1.34), C1-1329: I.A +
(A.3.15) + (A.1.34), C1-1330: I.A + (A.3.16) + (A.1.34), C1-1331: I.A + (A.3.17) + (A.1.34), C1-
1332: I.A + (A.3.18) + (A.1.34), C1-1333: I.A + (A.3.19) + (A.1.34), C1-1334: I.A + (A.3.20) +
(A.1.34), C1-1335: I.A + (A.3.21) + (A.1.34), C1-1336: I.A + (A.3.22) + (A.1.34), C1-1337: I.A +
(A.3.23) + (A.1.34), C1-1338: I.A + (A.3.24) + (A.1.34), C1-1339: I.A + (A.3.25) + (A.1.34), C1-
1340: I.A + (A.3.26) + (A.1.34), C1-1341: I.A + (A.3.27) + (A.1.34), C1-1342: I.A + (A.3.28) +
(A.1.34), C1-1343: I.A + (A.3.29) + (A.1.34), C1-1344: I.A + (A.3.31) + (A.1.34), C1-1345: I.A +
(A.3.32) + (A.1.34), C1-1346: I.A + (A.3.33) + (A.1.34), C1-1347: I.A + (A.3.34) + (A.1.34), C1-
1348: I.A + (A.3.35) + (A.1.34), C1-1349: I.A + (A.3.36) + (A.1.34), C1-1350: I.A + (A.3.37) +
(A.1.34), C1-1351: I.A + (A.3.38) + (A.1.34), C1-1352: I.A + (A.3.39) + (A.1.34), C1-1353: I.A +
(A.4.5) + (A.1.34), C1-1354: I.A + (A.4.11) + (A.1.34), C1-1355: I.A + (B.1.4) + (A.1.34), C1-
1356: I.A + (B.1.5) + (A.1.34), C1-1357: I.A + (B.1.8.) + (A.1.34), C1-1358: I.A + (B.1.10) +
(A.1.34), C1-1359: I.A + (B.1.11) + (A.1.34), C1-1360: I.A + (B.1.12) + (A.1.34), C1-1361: I.A +
(B.1.13) + (A.1.34), C1-1362: I.A + (B.1.17) + (A.1.34), C1-1363: I.A + (B.1.18) + (A.1.34), C1-
1364: I.A + (B.1.21) + (A.1.34), C1-1365: I.A + (B.1.22) + (A.1.34), C1-1366: I.A + (B.1.23) +
(A.1.34), C1-1367: I.A + (B.1.25) + (A.1.34), C1-1368: I.A + (B.1.26) + (A.1.34), C1-1369: I.A +
(B.1.29) + (A.1.34), C1-1370: I.A + (B.1.33) + (A.1.34), C1-1371: I.A + (B.1.34) + (A.1.34), C1-
1372: I.A + (B.1.37) + (A.1.34), C1-1373: I.A + (B.1.38) + (A.1.34), C1-1374: I.A + (B.1.43) +
(A.1.34), C1-1375: I.A + (B.1.46) + (A.1.34), C1-1376: I.A + (B.2.4) + (A.1.34), C1-1377: I.A +
(B.2.5) + (A.1.34), C1-1378: I.A + (B.2.6) + (A.1.34), C1-1379: I.A + (B.2.8) + (A.1.34), C1-1380:
I.A + (C.1.1) + (A.1.34), C1-1381: I.A + (C.1.2) + (A.1.34), C1-1382: I.A + (C.1.4) + (A.1.34), C1-
1383: I.A + (C.1.5) + (A.1.34), C1-1384: I.A + (C.2.6) + (A.1.34), C1-1385: I.A + (C.2.7) +
(A.1.34), C1-1386: I.A + (C.2.8) + (A.1.34), C1-1387: I.A + (D.1.1) + (A.1.34), C1-1388: I.A +
(D.1.2) + (A.1.34), C1-1389: I.A + (D.1.5) + (A.1.34), C1-1390: I.A + (D.2.4) + (A.1.34), C1-
1391: I.A + (D.2.6) + (A.1.34), C1-1392: I.A + (D.2.7) + (A.1.34), C1-1393: I.A + (E.1.1) +
(A.1.34), C1-1394: I.A + (E.1.3) + (A.1.34), C1-1395: I.A + (E.2.2) + (A.1.34), C1-1396: I.A +
(E.2.3) + (A.1.34), C1-1397: I.A + (F.1.2) + (A.1.34), C1-1398: I.A + (F.1.4) + (A.1.34), C1-1399:
I.A + (F.1.5) + (A.1.34), C1-1400: I.A + (G.3.1) + (A.1.34), C1-1401: I.A + (G.3.3) + (A.1.34), C1-
1402: I.A + (G.3.6) + (A.1.34), C1-1403: I.A + (G.5.1) + (A.1.34), C1-1404: I.A + (G.5.2) +
(A.1.34), C1-1405: I.A + (G.5.3) + (A.1.34), C1-1406: I.A + (G.5.4) + (A.1.34), C1-1407: I.A +
(G.5.5) + (A.1.34), C1-1408: I.A + (G.5.6) + (A.1.34), C1-1409: I.A + (G.5.7) + (A.1.34), C1-
1410: I.A + (G.5.8) + (A.1.34), C1-1411: I.A + (G.5.9) + (A.1.34), C1-1412: I.A + (G.5.10) +
(A.1.34), C1-1413: I.A + (G.5.11) + (A.1.34), C1-1414: I.A + (H.1.5) + (A.1.34), C1-1415: I.A +
(H.1.7) + (A.1.34), C1-1416: I.A + (H.2.2) + (A.1.34), C1-1417: I.A + (H.2.3) + (A.1.34), C1-
1418: I.A + (H.2.5) + (A.1.34), C1-1419: I.A + (H.2.7) + (A.1.34), C1-1420: I.A + (H.2.8) +
(A.1.34), C1-1421: I.A + (H.3.2) + (A.1.34), C1-1422: I.A + (H.3.4) + (A.1.34), C1-1423: I.A +
(H.3.5) + (A.1.34), C1-1424: I.A + (H.4.9) + (A.1.34), C1-1425: I.A + (H.4.10) + (A.1.34), C1-
1426: I.A + (I.2.2) + (A.1.34), C1-1427: I.A + (I.2.5) + (A.1.34), C1-1428: I.A + (J.1.2) + (A.1.34),
C1-1429: I.A + (J.1.5) + (A.1.34), C1-1430: I.A + (J.1.8) + (A.1.34), C1-1431: I.A + (J.1.11) +
(A.1.34), C1-1432: I.A + (J.1.12) + (A.1.34), C1-1433: I.A + (K.1.3) + (A.1.34), C1-1434: I.A +
(K.1.29) + (A.1.34), C1-1435: I.A + (K.1.30) + (A.1.34), C1-1436: I.A + (K.1.31) + (A.1.34), C1-
1437: I.A + (K.1.32) + (A.1.34), C1-1438: I.A + (K.1.41) + (A.1.34), C1-1439: I.A + (K.1.42) +
(A.1.34), C1-1440: I.A + (K.1.44) + (A.1.34), C1-1441: I.A + (K.1.45) + (A.1.34), C1-1442: I.A +
(K.1.47) + (A.1.34), C1-1443: I.A + (K.1.49) + (A.1.34), C1-1444: I.A + (K.1.53) + (A.1.34), C1-
1445: I.A + (K.1.54) + (A.1.34), C1-1446: I.A + (A.1.4) + (A.2.4), C1-1447: I.A + (A.1.12) +
(A.2.4), C1-1448: I.A + (A.1.21) + (A.2.4), C1-1449: I.A + (A.1,21a) + (A.2.4), C1-1450: I.A +
(A.1.24) + (A.2.4), C1-1451: I.A + (A.1.26) + (A.2.4), C1-1452: I.A + (A.1.27) + (A.2.4), C1-1453:
I.A + (A.1.30) + (A.2.4), C1-1454: I.A + (A.1.31) + (A.2.4), C1-1455: I.A + (A.1.32) + (A.2.4), C1-
1456: I.A + (A.1.35) + (A.2.4), C1-1457: I.A + (A.2.1) + (A.2.4), C1-1458: I.A + (A.2.3) + (A.2.4),
C1-1459: I.A + (A.2.5) + (A.2.4), C1-1460: I.A + (A.2.6) + (A.2.4), C1-1461: I.A + (A.3.2) +
(A.2.4), C1-1462: I.A + (A.3.3) + (A.2.4), C1-1463: I.A + (A.3.4) + (A.2.4), C1-1464: I.A + (A.3.7) +
(A.2.4), C1-1465: I.A + (A.3.9) + (A.2.4), C1-1466: I.A + (A.3.11) + (A.2.4), C1-1467: I.A +
(A.3.12) + (A.2.4), C1-1468: I.A + (A.3.15) + (A.2.4), C1-1469: I.A + (A.3.16) + (A.2.4), C1-1470:
I.A + (A.3.17) + (A.2.4), C1-1471: I.A + (A.3.18) + (A.2.4), C1-1472: I.A + (A.3.19) + (A.2.4), C1-
1473: I.A + (A.3.20) + (A.2.4), C1-1474: I.A + (A.3.21) + (A.2.4), C1-1475: I.A + (A.3.22) +
(A.2.4), C1-1476: I.A + (A.3.23) + (A.2.4), C1-1477: I.A + (A.3.24) + (A.2.4), C1-1478: I.A +
(A.3.25) + (A.2.4), C1-1479: I.A + (A.3.26) + (A.2.4), C1-1480: I.A + (A.3.27) + (A.2.4), C1-1481:
I.A + (A.3.28) + (A.2.4), C1-1482: I.A + (A.3.29) + (A.2.4), C1-1483: I.A + (A.3.31) + (A.2.4), C1-
1484: I.A + (A.3.32) + (A.2.4), C1-1485: I.A + (A.3.33) + (A.2.4), C1-1486: I.A + (A.3.34) +
(A.2.4), C1-1487: I.A + (A.3.35) + (A.2.4), C1-1488: I.A + (A.3.36) + (A.2.4), C1-1489: I.A +
(A.3.37) + (A.2.4), C1-1490: I.A + (A.3.38) + (A.2.4), C1-1491: I.A + (A.3.39) + (A.2.4), C1-1492:
I.A + (A.4.5) + (A.2.4), C1-1493: I.A + (A.4.11) + (A.2.4), C1-1494: I.A + (B.1.4) + (A.2.4), C1-
1495: I.A + (B.1.5) + (A.2.4), C1-1496: I.A + (B.1.8.) + (A.2.4), C1-1497: I.A + (B.1.10) + (A.2.4),
C1-1498: I.A + (B.1.11) + (A.2.4), C1-1499: I.A + (B.1.12) + (A.2.4), C1-1500: I.A + (B.1.13) +
(A.2.4), C1-1501: I.A + (B.1.17) + (A.2.4), C1-1502: I.A + (B.1.18) + (A.2.4), C1-1503: I.A +
(B.1.21) + (A.2.4), C1-1504: I.A + (B.1.22) + (A.2.4), C1-1505: I.A + (B.1.23) + (A.2.4), C1-1506:
I.A + (B.1.25) + (A.2.4), C1-1507: I.A + (B.1.26) + (A.2.4), C1-1508: I.A + (B.1.29) + (A.2.4), C1-
1509: I.A + (B.1.33) + (A.2.4), C1-1510: I.A + (B.1.34) + (A.2.4), C1-1511: I.A + (B.1.37) +
(A.2.4), C1-1512: I.A + (B.1.38) + (A.2.4), C1-1513: I.A + (B.1.43) + (A.2.4), C1-1514: I.A +
(B.1.46) + (A.2.4), C1-1515: I.A + (B.2.4) + (A.2.4), C1-1516: I.A + (B.2.5) + (A.2.4), C1-1517:
I.A + (B.2.6) + (A.2.4), C1-1518: I.A + (B.2.8) + (A.2.4), C1-1519: I.A + (C.1.1) + (A.2.4), C1-
1520: I.A + (C.1.2) + (A.2.4), C1-1521: I.A + (C.1.4) + (A.2.4), C1-1522: I.A + (C.1.5) + (A.2.4),
C1-1523: I.A + (C.2.6) + (A.2.4), C1-1524: I.A + (C.2.7) + (A.2.4), C1-1525: I.A + (C.2.8) +
(A.2.4), C1-1526: I.A + (D.1.1) + (A.2.4), C1-1527: I.A + (D.1.2) + (A.2.4), C1-1528: I.A + (D.1.5) +
(A.2.4), C1-1529: I.A + (D.2.4) + (A.2.4), C1-1530: I.A + (D.2.6) + (A.2.4), C1-1531: I.A +
(D.2.7) + (A.2.4), C1-1532: I.A + (E.1.1) + (A.2.4), C1-1533: I.A + (E.1.3) + (A.2.4), C1-1534: I.A +
(E.2.2) + (A.2.4), C1-1535: I.A + (E.2.3) + (A.2.4), C1-1536: I.A + (F.1.2) + (A.2.4), C1-1537:
I.A + (F.1.4) + (A.2.4), C1-1538: I.A + (F.1.5) + (A.2.4), C1-1539: I.A + (G.3.1) + (A.2.4), C1-
1540: I.A + (G.3.3) + (A.2.4), C1-1541: I.A + (G.3.6) + (A.2.4), C1-1542: I.A + (G.5.1) + (A.2.4),
C1-1543: I.A + (G.5.2) + (A.2.4), C1-1544: I.A + (G.5.3) + (A.2.4), C1-1545: I.A + (G.5.4) +
(A.2.4), C1-1546: I.A + (G.5.5) + (A.2.4), C1-1547: I.A + (G.5.6) + (A.2.4), C1-1548: I.A +
(G.5.7) + (A.2.4), C1-1549: I.A + (G.5.8) + (A.2.4), C1-1550: I.A + (G.5.9) + (A.2.4), C1-1551:
I.A + (G.5.10) + (A.2.4), C1-1552: I.A + (G.5.11) + (A.2.4), C1-1553: I.A + (H.1.5) + (A.2.4), C1-
1554: I.A + (H.1.7) + (A.2.4), C1-1555: I.A + (H.2.2) + (A.2.4), C1-1556: I.A + (H.2.3) + (A.2.4),
C1-1557: I.A + (H.2.5) + (A.2.4), C1-1558: I.A + (H.2.7) + (A.2.4), C1-1559: I.A + (H.2.8) +
(A.2.4), C1-1560: I.A + (H.3.2) + (A.2.4), C1-1561: I.A + (H.3.4) + (A.2.4), C1-1562: I.A + (H.3.5) +
(A.2.4), C1-1563: I.A + (H.4.9) + (A.2.4), C1-1564: I.A + (H.4.10) + (A.2.4), C1-1565: I.A +
(I.2.2) + (A.2.4), C1-1566: I.A + (I.2.5) + (A.2.4), C1-1567: I.A + (J.1.2) + (A.2.4), C1-1568: I.A +
(J.1.5) + (A.2.4), C1-1569: I.A + (J.1.8) + (A.2.4), C1-1570: I.A + (J.1.11) + (A.2.4), C1-1571: I.A +
(J.1.12) + (A.2.4), C1-1572: I.A + (K.1.3) + (A.2.4), C1-1573: I.A + (K.1.29) + (A.2.4), C1-
1574: I.A + (K.1.30) + (A.2.4), C1-1575: I.A + (K.1.31) + (A.2.4), C1-1576: I.A + (K.1.32) +
(A.2.4), C1-1577: I.A + (K.1.41) + (A.2.4), C1-1578: I.A + (K.1.42) + (A.2.4), C1-1579: I.A +
(K.1.44) + (A.2.4), C1-1580: I.A + (K.1.45) + (A.2.4), C1-1581: I.A + (K.1.47) + (A.2.4), C1-1582:
I.A + (K.1.49) + (A.2.4), C1-1583: I.A + (K.1.53) + (A.2.4), C1-1584: I.A + (K.1.54) + (A.2.4), C1-
1585: I.A + (A.1.12) + (A.2.6), C1-1586: I.A + (A.1.27) + (A.2.6), C1-1587: I.A + (A.1.35) +
(A.2.6), C1-1588: I.A + (A.2.1) + (A.2.6), C1-1589: I.A + (A.2.3) + (A.2.6), C1-1590: I.A + (A.3.2) +
(A.2.6), C1-1591: I.A + (A.3.3) + (A.2.6), C1-1592: I.A + (A.3.4) + (A.2.6), C1-1593: I.A +
(A.3.7) + (A.2.6), C1-1594: I.A + (A.3.9) + (A.2.6), C1-1595: I.A + (A.3.12) + (A.2.6), C1-1596:
I.A + (A.3.15) + (A.2.6), C1-1597: I.A + (A.3.16) + (A.2.6), C1-1598: I.A + (A.3.18) + (A.2.6), C1-
1599: I.A + (A.3.20) + (A.2.6), C1-1600: I.A + (A.3.21) + (A.2.6), C1-1601: I.A + (A.3.22) +
(A.2.6), C1-1602: I.A + (A.3.23) + (A.2.6), C1-1603: I.A + (A.3.24) + (A.2.6), C1-1604: I.A +
(A.3.25) + (A.2.6), C1-1605: I.A + (A.3.26) + (A.2.6), C1-1606: I.A + (A.3.27) + (A.2.6), C1-1607:
I.A + (A.3.28) + (A.2.6), C1-1608: I.A + (A.3.29) + (A.2.6), C1-1609: I.A + (A.3.31) + (A.2.6), C1-
1610: I.A + (A.3.32) + (A.2.6), C1-1611: I.A + (A.3.33) + (A.2.6), C1-1612: I.A + (A.3.34) +
(A.2.6), C1-1613: I.A + (A.3.35) + (A.2.6), C1-1614: I.A + (A.3.36) + (A.2.6), C1-1615: I.A +
(A.3.37) + (A.2.6), C1-1616: I.A + (A.3.38) + (A.2.6), C1-1617: I.A + (A.3.39) + (A.2.6), C1-1618:
I.A + (A.4.5) + (A.2.6), C1-1619: I.A + (A.4.11) + (A.2.6), C1-1620: I.A + (B.1.4) + (A.2.6), C1-
1621: I.A + (B.1.5) + (A.2.6), C1-1622: I.A + (B.1.8.) + (A.2.6), C1-1623: I.A + (B.1.10) + (A.2.6),
C1-1624: I.A + (B.1.11) + (A.2.6), C1-1625: I.A + (B.1.12) + (A.2.6), C1-1626: I.A + (B.1.13) +
(A.2.6), C1-1627: I.A + (B.1.17) + (A.2.6), C1-1628: I.A + (B.1.18) + (A.2.6), C1-1629: I.A +
(B.1.21) + (A.2.6), C1-1630: I.A + (B.1.22) + (A.2.6), C1-1631: I.A + (B.1.23) + (A.2.6), C1-1632:
I.A + (B.1.25) + (A.2.6), C1-1633: I.A + (B.1.26) + (A.2.6), C1-1634: I.A + (B.1.29) + (A.2.6), C1-
1635: I.A + (B.1.33) + (A.2.6), C1-1636: I.A + (B.1.34) + (A.2.6), C1-1637: I.A + (B.1.37) +
(A.2.6), C1-1638: I.A + (B.1.38) + (A.2.6), C1-1639: I.A + (B.1.43) + (A.2.6), C1-1640: I.A +
(B.1.46) + (A.2.6), C1-1641: I.A + (B.2.4) + (A.2.6), C1-1642: I.A + (B.2.5) + (A.2.6), C1-1643:
I.A + (B.2.6) + (A.2.6), C1-1644: I.A + (B.2.8) + (A.2.6), C1-1645: I.A + (C.1.1) + (A.2.6), C1-
1646: I.A + (C.1.2) + (A.2.6), C1-1647: I.A + (C.1.4) + (A.2.6), C1-1648: I.A + (C.1.5) + (A.2.6),
C1-1649: I.A + (C.2.6) + (A.2.6), C1-1650: I.A + (C.2.7) + (A.2.6), C1-1651: I.A + (C.2.8) +
(A.2.6), C1-1652: I.A + (D.1.1) + (A.2.6), C1-1653: I.A + (D.1.2) + (A.2.6), C1-1654: I.A + (D.1.5) +
(A.2.6), C1-1655: I.A + (D.2.4) + (A.2.6), C1-1656: I.A + (D.2.6) + (A.2.6), C1-1657: I.A +
(D.2.7) + (A.2.6), C1-1658: I.A + (E.1.1) + (A.2.6), C1-1659: I.A + (E.1.3) + (A.2.6), C1-1660: I.A +
(E.2.2) + (A.2.6), C1-1661: I.A + (E.2.3) + (A.2.6), C1-1662: I.A + (F.1.2) + (A.2.6), C1-1663:
I.A + (F.1.4) + (A.2.6), C1-1664: I.A + (F.1.5) + (A.2.6), C1-1665: I.A + (G.3.1) + (A.2.6), C1-
1666: I.A + (G.3.3) + (A.2.6), C1-1667: I.A + (G.3.6) + (A.2.6), C1-1668: I.A + (G.5.1) + (A.2.6),
C1-1669: I.A + (G.5.2) + (A.2.6), C1-1670: I.A + (G.5.3) + (A.2.6), C1-1671: I.A + (G.5.4) +
(A.2.6), C1-1672: I.A + (G.5.5) + (A.2.6), C1-1673: I.A + (G.5.6) + (A.2.6), C1-1674: I.A +
(G.5.7) + (A.2.6), C1-1675: I.A + (G.5.8) + (A.2.6), C1-1676: I.A + (G.5.9) + (A.2.6), C1-1677:
I.A + (G.5.10) + (A.2.6), C1-1678: I.A + (G.5.11) + (A.2.6), C1-1679: I.A + (H.1.5) + (A.2.6), C1-
1680: I.A + (H.1.7) + (A.2.6), C1-1681: I.A + (H.2.2) + (A.2.6), C1-1682: I.A + (H.2.3) + (A.2.6),
C1-1683: I.A + (H.2.5) + (A.2.6), C1-1684: I.A + (H.2.7) + (A.2.6), C1-1685: I.A + (H.2.8) +
(A.2.6), C1-1686: I.A + (H.3.2) + (A.2.6), C1-1687: I.A + (H.3.4) + (A.2.6), C1-1688: I.A + (H.3.5) +
(A.2.6), C1-1689: I.A + (H.4.9) + (A.2.6), C1-1690: I.A + (H.4.10) + (A.2.6), C1-1691: I.A +
(I.2.2) + (A.2.6), C1-1692: I.A + (I.2.5) + (A.2.6), C1-1693: I.A + (J.1.2) + (A.2.6), C1-1694: I.A +
(J.1.5) + (A.2.6), C1-1695: I.A + (J.1.8) + (A.2.6), C1-1696: I.A + (J.1.11) + (A.2.6), C1-1697: I.A +
(J.1.12) + (A.2.6), C1-1698: I.A + (K.1.3) + (A.2.6), C1-1699: I.A + (K.1.29) + (A.2.6), C1-
1700: I.A + (K.1.30) + (A.2.6), C1-1701: I.A + (K.1.31) + (A.2.6), C1-1702: I.A + (K.1.32) +
(A.2.6), C1-1703: I.A + (K.1.41) + (A.2.6), C1-1704: I.A + (K.1.42) + (A.2.6), C1-1705: I.A +
(K.1.44) + (A.2.6), C1-1706: I.A + (K.1.45) + (A.2.6), C1-1707: I.A + (K.1.47) + (A.2.6), C1-1708:
I.A + (K.1.49) + (A.2.6), C1-1709: I.A + (K.1.53) + (A.2.6), C1-1710: I.A + (K.1.54) + (A.2.6), C1-
1711: I.A + (A.1.4) + (A.3.2), C1-1712: I.A + (A.1.12) + (A.3.2), C1-1713: I.A + (A.1.21) +
(A.3.2), C1-1714: I.A + (A.1,21a) + (A.3.2), C1-1715: I.A + (A.1.24) + (A.3.2), C1-1716: I.A +
(A.1.26) + (A.3.2), C1-1717: I.A + (A.1.27) + (A.3.2), C1-1718: I.A + (A.1.30) + (A.3.2), C1-1719:
I.A + (A.1.31) + (A.3.2), C1-1720: I.A + (A.1.32) + (A.3.2), C1-1721: I.A + (A.1.35) + (A.3.2), C1-
1722: I.A + (A.2.1) + (A.3.2), C1-1723: I.A + (A.2.3) + (A.3.2), C1-1724: I.A + (A.2.5) + (A.3.2),
C1-1725: I.A + (A.3.3) + (A.3.2), C1-1726: I.A + (A.3.4) + (A.3.2), C1-1727: I.A + (A.3.7) +
(A.3.2), C1-1728: I.A + (A.3.9) + (A.3.2), C1-1729: I.A + (A.3.11) + (A.3.2), C1-1730: I.A +
(A.3.12) + (A.3.2), C1-1731: I.A + (A.3.15) + (A.3.2), C1-1732: I.A + (A.3.16) + (A.3.2), C1-1733:
I.A + (A.3.17) + (A.3.2), C1-1734: I.A + (A.3.18) + (A.3.2), C1-1735: I.A + (A.3.19) + (A.3.2), C1-
1736: I.A + (A.3.20) + (A.3.2), C1-1737: I.A + (A.3.21) + (A.3.2), C1-1738: I.A + (A.3.22) +
(A.3.2), C1-1739: I.A + (A.3.23) + (A.3.2), C1-1740: I.A + (A.3.24) + (A.3.2), C1-1741: I.A +
(A.3.25) + (A.3.2), C1-1742: I.A + (A.3.26) + (A.3.2), C1-1743: I.A + (A.3.27) + (A.3.2), C1-1744:
I.A + (A.3.28) + (A.3.2), C1-1745: I.A + (A.3.29) + (A.3.2), C1-1746: I.A + (A.3.31) + (A.3.2), C1-
1747: I.A + (A.3.32) + (A.3.2), C1-1748: I.A + (A.3.33) + (A.3.2), C1-1749: I.A + (A.3.34) +
(A.3.2), C1-1750: I.A + (A.3.35) + (A.3.2), C1-1751: I.A + (A.3.36) + (A.3.2), C1-1752: I.A +
(A.3.37) + (A.3.2), C1-1753: I.A + (A.3.38) + (A.3.2), C1-1754: I.A + (A.3.39) + (A.3.2), C1-1755:
I.A + (A.4.5) + (A.3.2), C1-1756: I.A + (A.4.11) + (A.3.2), C1-1757: I.A + (B.1.4) + (A.3.2), C1-
1758: I.A + (B.1.5) + (A.3.2), C1-1759: I.A + (B.1.8.) + (A.3.2), C1-1760: I.A + (B.1.10) + (A.3.2),
C1-1761: I.A + (B.1.11) + (A.3.2), C1-1762: I.A + (B.1.12) + (A.3.2), C1-1763: I.A + (B.1.13) +
(A.3.2), C1-1764: I.A + (B.1.17) + (A.3.2), C1-1765: I.A + (B.1.18) + (A.3.2), C1-1766: I.A +
(B.1.21) + (A.3.2), C1-1767: I.A + (B.1.22) + (A.3.2), C1-1768: I.A + (B.1.23) + (A.3.2), C1-1769:
I.A + (B.1.25) + (A.3.2), C1-1770: I.A + (B.1.26) + (A.3.2), C1-1771: I.A + (B.1.29) + (A.3.2), C1-
1772: I.A + (B.1.33) + (A.3.2), C1-1773: I.A + (B.1.34) + (A.3.2), C1-1774: I.A + (B.1.37) +
(A.3.2), C1-1775: I.A + (B.1.38) + (A.3.2), C1-1776: I.A + (B.1.43) + (A.3.2), C1-1777: I.A +
(B.1.46) + (A.3.2), C1-1778: I.A + (B.2.4) + (A.3.2), C1-1779: I.A + (B.2.5) + (A.3.2), C1-1780:
I.A + (B.2.6) + (A.3.2), C1-1781: I.A + (B.2.8) + (A.3.2), C1-1782: I.A + (C.1.1) + (A.3.2), C1-
1783: I.A + (C.1.2) + (A.3.2), C1-1784: I.A + (C.1.4) + (A.3.2), C1-1785: I.A + (C.1.5) + (A.3.2),
C1-1786: I.A + (C.2.6) + (A.3.2), C1-1787: I.A + (C.2.7) + (A.3.2), C1-1788: I.A + (C.2.8) +
(A.3.2), C1-1789: I.A + (D.1.1) + (A.3.2), C1-1790: I.A + (D.1.2) + (A.3.2), C1-1791: I.A + (D.1.5) +
(A.3.2), C1-1792: I.A + (D.2.4) + (A.3.2), C1-1793: I.A + (D.2.6) + (A.3.2), C1-1794: I.A +
(D.2.7) + (A.3.2), C1-1795: I.A + (E.1.1) + (A.3.2), C1-1796: I.A + (E.1.3) + (A.3.2), C1-1797: I.A +
(E.2.2) + (A.3.2), C1-1798: I.A + (E.2.3) + (A.3.2), C1-1799: I.A + (F.1.2) + (A.3.2), C1-1800:
I.A + (F.1.4) + (A.3.2), C1-1801: I.A + (F.1.5) + (A.3.2), C1-1802: I.A + (G.3.1) + (A.3.2), C1-
1803: I.A + (G.3.3) + (A.3.2), C1-1804: I.A + (G.3.6) + (A.3.2), C1-1805: I.A + (G.5.1) + (A.3.2),
C1-1806: I.A + (G.5.2) + (A.3.2), C1-1807: I.A + (G.5.3) + (A.3.2), C1-1808: I.A + (G.5.4) +
(A.3.2), C1-1809: I.A + (G.5.5) + (A.3.2), C1-1810: I.A + (G.5.6) + (A.3.2), C1-1811: I.A +
(G.5.7) + (A.3.2), C1-1812: I.A + (G.5.8) + (A.3.2), C1-1813: I.A + (G.5.9) + (A.3.2), C1-1814:
I.A + (G.5.10) + (A.3.2), C1-1815: I.A + (G.5.11) + (A.3.2), C1-1816: I.A + (H.1.5) + (A.3.2), C1-
1817: I.A + (H.1.7) + (A.3.2), C1-1818: I.A + (H.2.2) + (A.3.2), C1-1819: I.A + (H.2.3) + (A.3.2),
C1-1820: I.A + (H.2.5) + (A.3.2), C1-1821: I.A + (H.2.7) + (A.3.2), C1-1822: I.A + (H.2.8) +
(A.3.2), C1-1823: I.A + (H.3.2) + (A.3.2), C1-1824: I.A + (H.3.4) + (A.3.2), C1-1825: I.A + (H.3.5) +
(A.3.2), C1-1826: I.A + (H.4.9) + (A.3.2), C1-1827: I.A + (H.4.10) + (A.3.2), C1-1828: I.A +
(I.2.2) + (A.3.2), C1-1829: I.A + (I.2.5) + (A.3.2), C1-1830: I.A + (J.1.2) + (A.3.2), C1-1831: I.A +
(J.1.5) + (A.3.2), C1-1832: I.A + (J.1.8) + (A.3.2), C1-1833: I.A + (J.1.11) + (A.3.2), C1-1834: I.A +
(J.1.12) + (A.3.2), C1-1835: I.A + (K.1.3) + (A.3.2), C1-1836: I.A + (K.1.29) + (A.3.2), C1-
1837: I.A + (K.1.30) + (A.3.2), C1-1838: I.A + (K.1.31) + (A.3.2), C1-1839: I.A + (K.1.32) +
(A.3.2), C1-1840: I.A + (K.1.41) + (A.3.2), C1-1841: I.A + (K.1.42) + (A.3.2), C1-1842: I.A +
(K.1.44) + (A.3.2), C1-1843: I.A + (K.1.45) + (A.3.2), C1-1844: I.A + (K.1.47) + (A.3.2), C1-1845:
I.A + (K.1.49) + (A.3.2), C1-1846: I.A + (K.1.53) + (A.3.2), C1-1847: I.A + (K.1.54) + (A.3.2), C1-
1848: I.A + (A.1.4) + (A.3.3), C1-1849: I.A + (A.1.12) + (A.3.3), C1-1850: I.A + (A.1.21) +
(A.3.3), C1-1851: I.A + (A.1,21a) + (A.3.3), C1-1852: I.A + (A.1.24) + (A.3.3), C1-1853: I.A +
(A.1.26) + (A.3.3), C1-1854: I.A + (A.1.27) + (A.3.3), C1-1855: I.A + (A.1.30) + (A.3.3), C1-1856:
I.A + (A.1.31) + (A.3.3), C1-1857: I.A + (A.1.32) + (A.3.3), C1-1858: I.A + (A.1.35) + (A.3.3), C1-
1859: I.A + (A.2.1) + (A.3.3), C1-1860: I.A + (A.2.3) + (A.3.3), C1-1861: I.A + (A.2.5) + (A.3.3),
C1-1862: I.A + (A.3.4) + (A.3.3), C1-1863: I.A + (A.3.7) + (A.3.3), C1-1864: I.A + (A.3.9) +
(A.3.3), C1-1865: I.A + (A.3.11) + (A.3.3), C1-1866: I.A + (A.3.12) + (A.3.3), C1-1867: I.A +
(A.3.15) + (A.3.3), C1-1868: I.A + (A.3.16) + (A.3.3), C1-1869: I.A + (A.3.17) + (A.3.3), C1-1870:
I.A + (A.3.18) + (A.3.3), C1-1871: I.A + (A.3.19) + (A.3.3), C1-1872: I.A + (A.3.20) + (A.3.3), C1-
1873: I.A + (A.3.21) + (A.3.3), C1-1874: I.A + (A.3.22) + (A.3.3), C1-1875: I.A + (A.3.23) +
(A.3.3), C1-1876: I.A + (A.3.24) + (A.3.3), C1-1877: I.A + (A.3.25) + (A.3.3), C1-1878: I.A +
(A.3.26) + (A.3.3), C1-1879: I.A + (A.3.27) + (A.3.3), C1-1880: I.A + (A.3.28) + (A.3.3), C1-1881:
I.A + (A.3.29) + (A.3.3), C1-1882: I.A + (A.3.31) + (A.3.3), C1-1883: I.A + (A.3.32) + (A.3.3), C1-
1884: I.A + (A.3.33) + (A.3.3), C1-1885: I.A + (A.3.34) + (A.3.3), C1-1886: I.A + (A.3.35) +
(A.3.3), C1-1887: I.A + (A.3.36) + (A.3.3), C1-1888: I.A + (A.3.37) + (A.3.3), C1-1889: I.A +
(A.3.38) + (A.3.3), C1-1890: I.A + (A.3.39) + (A.3.3), C1-1891: I.A + (A.4.5) + (A.3.3), C1-1892:
I.A + (A.4.11) + (A.3.3), C1-1893: I.A + (B.1.4) + (A.3.3), C1-1894: I.A + (B.1.5) + (A.3.3), C1-
1895: I.A + (B.1.8.) + (A.3.3), C1-1896: I.A + (B.1.10) + (A.3.3), C1-1897: I.A + (B.1.11) +
(A.3.3), C1-1898: I.A + (B.1.12) + (A.3.3), C1-1899: I.A + (B.1.13) + (A.3.3), C1-1900: I.A +
(B.1.17) + (A.3.3), C1-1901: I.A + (B.1.18) + (A.3.3), C1-1902: I.A + (B.1.21) + (A.3.3), C1-1903:
I.A + (B.1.22) + (A.3.3), C1-1904: I.A + (B.1.23) + (A.3.3), C1-1905: I.A + (B.1.25) + (A.3.3), C1-
1906: I.A + (B.1.26) + (A.3.3), C1-1907: I.A + (B.1.29) + (A.3.3), C1-1908: I.A + (B.1.33) +
(A.3.3), C1-1909: I.A + (B.1.34) + (A.3.3), C1-1910: I.A + (B.1.37) + (A.3.3), C1-1911: I.A +
(B.1.38) + (A.3.3), C1-1912: I.A + (B.1.43) + (A.3.3), C1-1913: I.A + (B.1.46) + (A.3.3), C1-1914:
I.A + (B.2.4) + (A.3.3), C1-1915: I.A + (B.2.5) + (A.3.3), C1-1916: I.A + (B.2.6) + (A.3.3), C1-
1917: I.A + (B.2.8) + (A.3.3), C1-1918: I.A + (C.1.1) + (A.3.3), C1-1919: I.A + (C.1.2) + (A.3.3),
C1-1920: I.A + (C.1.4) + (A.3.3), C1-1921: I.A + (C.1.5) + (A.3.3), C1-1922: I.A + (C.2.6) +
(A.3.3), C1-1923: I.A + (C.2.7) + (A.3.3), C1-1924: I.A + (C.2.8) + (A.3.3), C1-1925: I.A + (D.1.1) +
(A.3.3), C1-1926: I.A + (D.1.2) + (A.3.3), C1-1927: I.A + (D.1.5) + (A.3.3), C1-1928: I.A +
(D.2.4) + (A.3.3), C1-1929: I.A + (D.2.6) + (A.3.3), C1-1930: I.A + (D.2.7) + (A.3.3), C1-1931: I.A +
(E.1.1) + (A.3.3), C1-1932: I.A + (E.1.3) + (A.3.3), C1-1933: I.A + (E.2.2) + (A.3.3), C1-1934:
I.A + (E.2.3) + (A.3.3), C1-1935: I.A + (F.1.2) + (A.3.3), C1-1936: I.A + (F.1.4) + (A.3.3), C1-
1937: I.A + (F.1.5) + (A.3.3), C1-1938: I.A + (G.3.1) + (A.3.3), C1-1939: I.A + (G.3.3) + (A.3.3),
C1-1940: I.A + (G.3.6) + (A.3.3), C1-1941: I.A + (G.5.1) + (A.3.3), C1-1942: I.A + (G.5.2) +
(A.3.3), C1-1943: I.A + (G.5.3) + (A.3.3), C1-1944: I.A + (G.5.4) + (A.3.3), C1-1945: I.A +
(G.5.5) + (A.3.3), C1-1946: I.A + (G.5.6) + (A.3.3), C1-1947: I.A + (G.5.7) + (A.3.3), C1-1948:
I.A + (G.5.8) + (A.3.3), C1-1949: I.A + (G.5.9) + (A.3.3), C1-1950: I.A + (G.5.10) + (A.3.3), C1-
1951: I.A + (G.5.11) + (A.3.3), C1-1952: I.A + (H.1.5) + (A.3.3), C1-1953: I.A + (H.1.7) + (A.3.3),
C1-1954: I.A + (H.2.2) + (A.3.3), C1-1955: I.A + (H.2.3) + (A.3.3), C1-1956: I.A + (H.2.5) +
(A.3.3), C1-1957: I.A + (H.2.7) + (A.3.3), C1-1958: I.A + (H.2.8) + (A.3.3), C1-1959: I.A + (H.3.2) +
(A.3.3), C1-1960: I.A + (H.3.4) + (A.3.3), C1-1961: I.A + (H.3.5) + (A.3.3), C1-1962: I.A +
(H.4.9) + (A.3.3), C1-1963: I.A + (H.4.10) + (A.3.3), C1-1964: I.A + (I.2.2) + (A.3.3), C1-1965: I.A +
(I.2.5) + (A.3.3), C1-1966: I.A + (J.1.2) + (A.3.3), C1-1967: I.A + (J.1.5) + (A.3.3), C1-1968: I.A +
(J.1.8) + (A.3.3), C1-1969: I.A + (J.1.11) + (A.3.3), C1-1970: I.A + (J.1.12) + (A.3.3), C1-1971:
I.A + (K.1.3) + (A.3.3), C1-1972: I.A + (K.1.29) + (A.3.3), C1-1973: I.A + (K.1.30) + (A.3.3), C1-
1974: I.A + (K.1.31) + (A.3.3), C1-1975: I.A + (K.1.32) + (A.3.3), C1-1976: I.A + (K.1.41) +
(A.3.3), C1-1977: I.A + (K.1.42) + (A.3.3), C1-1978: I.A + (K.1.44) + (A.3.3), C1-1979: I.A +
(K.1.45) + (A.3.3), C1-1980: I.A + (K.1.47) + (A.3.3), C1-1981: I.A + (K.1.49) + (A.3.3), C1-1982:
I.A + (K.1.53) + (A.3.3), C1-1983: I.A + (K.1.54) + (A.3.3), C1-1984: I.A + (A.1.4) + (A.3.4), C1-
1985: I.A + (A.1.12) + (A.3.4), C1-1986: I.A + (A.1.21) + (A.3.4), C1-1987: I.A + (A.1,21a) +
(A.3.4), C1-1988: I.A + (A.1.24) + (A.3.4), C1-1989: I.A + (A.1.26) + (A.3.4), C1-1990: I.A +
(A.1.27) + (A.3.4), C1-1991: I.A + (A.1.30) + (A.3.4), C1-1992: I.A + (A.1.31) + (A.3.4), C1-1993:
I.A + (A.1.32) + (A.3.4), C1-1994: I.A + (A.1.35) + (A.3.4), C1-1995: I.A + (A.2.1) + (A.3.4), C1-
1996: I.A + (A.2.3) + (A.3.4), C1-1997: I.A + (A.2.5) + (A.3.4), C1-1998: I.A + (A.3.7) + (A.3.4),
C1-1999: I.A + (A.3.9) + (A.3.4), C1-2000: I.A + (A.3.11) + (A.3.4), C1-2001: I.A + (A.3.12) +
(A.3.4), C1-2002: I.A + (A.3.15) + (A.3.4), C1-2003: I.A + (A.3.16) + (A.3.4), C1-2004: I.A +
(A.3.17) + (A.3.4), C1-2005: I.A + (A.3.18) + (A.3.4), C1-2006: I.A + (A.3.19) + (A.3.4), C1-2007:
I.A + (A.3.20) + (A.3.4), C1-2008: I.A + (A.3.21) + (A.3.4), C1-2009: I.A + (A.3.22) + (A.3.4), C1-
2010: I.A + (A.3.23) + (A.3.4), C1-2011: I.A + (A.3.24) + (A.3.4), C1-2012: I.A + (A.3.25) +
(A.3.4), C1-2013: I.A + (A.3.26) + (A.3.4), C1-2014: I.A + (A.3.27) + (A.3.4), C1-2015: I.A +
(A.3.28) + (A.3.4), C1-2016: I.A + (A.3.29) + (A.3.4), C1-2017: I.A + (A.3.31) + (A.3.4), C1-2018:
I.A + (A.3.32) + (A.3.4), C1-2019: I.A + (A.3.33) + (A.3.4), C1-2020: I.A + (A.3.34) + (A.3.4), C1-
2021: I.A + (A.3.35) + (A.3.4), C1-2022: I.A + (A.3.36) + (A.3.4), C1-2023: I.A + (A.3.37) +
(A.3.4), C1-2024: I.A + (A.3.38) + (A.3.4), C1-2025: I.A + (A.3.39) + (A.3.4), C1-2026: I.A +
(A.4.5) + (A.3.4), C1-2027: I.A + (A.4.11) + (A.3.4), C1-2028: I.A + (B.1.4) + (A.3.4), C1-2029:
I.A + (B.1.5) + (A.3.4), C1-2030: I.A + (B.1.8.) + (A.3.4), C1-2031: I.A + (B.1.10) + (A.3.4), C1-
2032: I.A + (B.1.11) + (A.3.4), C1-2033: I.A + (B.1.12) + (A.3.4), C1-2034: I.A + (B.1.13) +
(A.3.4), C1-2035: I.A + (B.1.17) + (A.3.4), C1-2036: I.A + (B.1.18) + (A.3.4), C1-2037: I.A +
(B.1.21) + (A.3.4), C1-2038: I.A + (B.1.22) + (A.3.4), C1-2039: I.A + (B.1.23) + (A.3.4), C1-2040:
I.A + (B.1.25) + (A.3.4), C1-2041: I.A + (B.1.26) + (A.3.4), C1-2042: I.A + (B.1.29) + (A.3.4), C1-
2043: I.A + (B.1.33) + (A.3.4), C1-2044: I.A + (B.1.34) + (A.3.4), C1-2045: I.A + (B.1.37) +
(A.3.4), C1-2046: I.A + (B.1.38) + (A.3.4), C1-2047: I.A + (B.1.43) + (A.3.4), C1-2048: I.A +
(B.1.46) + (A.3.4), C1-2049: I.A + (B.2.4) + (A.3.4), C1-2050: I.A + (B.2.5) + (A.3.4), C1-2051:
I.A + (B.2.6) + (A.3.4), C1-2052: I.A + (B.2.8) + (A.3.4), C1-2053: I.A + (C.1.1) + (A.3.4), C1-
2054: I.A + (C.1.2) + (A.3.4), C1-2055: I.A + (C.1.4) + (A.3.4), C1-2056: I.A + (C.1.5) + (A.3.4),
C1-2057: I.A + (C.2.6) + (A.3.4), C1-2058: I.A + (C.2.7) + (A.3.4), C1-2059: I.A + (C.2.8) +
(A.3.4), C1-2060: I.A + (D.1.1) + (A.3.4), C1-2061: I.A + (D.1.2) + (A.3.4), C1-2062: I.A + (D.1.5) +
(A.3.4), C1-2063: I.A + (D.2.4) + (A.3.4), C1-2064: I.A + (D.2.6) + (A.3.4), C1-2065: I.A +
(D.2.7) + (A.3.4), C1-2066: I.A + (E.1.1) + (A.3.4), C1-2067: I.A + (E.1.3) + (A.3.4), C1-2068: I.A +
(E.2.2) + (A.3.4), C1-2069: I.A + (E.2.3) + (A.3.4), C1-2070: I.A + (F.1.2) + (A.3.4), C1-2071:
I.A + (F.1.4) + (A.3.4), C1-2072: I.A + (F.1.5) + (A.3.4), C1-2073: I.A + (G.3.1) + (A.3.4), C1-
2074: I.A + (G.3.3) + (A.3.4), C1-2075: I.A + (G.3.6) + (A.3.4), C1-2076: I.A + (G.5.1) + (A.3.4),
C1-2077: I.A + (G.5.2) + (A.3.4), C1-2078: I.A + (G.5.3) + (A.3.4), C1-2079: I.A + (G.5.4) +
(A.3.4), C1-2080: I.A + (G.5.5) + (A.3.4), C1-2081: I.A + (G.5.6) + (A.3.4), C1-2082: I.A +
(G.5.7) + (A.3.4), C1-2083: I.A + (G.5.8) + (A.3.4), C1-2084: I.A + (G.5.9) + (A.3.4), C1-2085:
I.A + (G.5.10) + (A.3.4), C1-2086: I.A + (G.5.11) + (A.3.4), C1-2087: I.A + (H.1.5) + (A.3.4), C1-
2088: I.A + (H.1.7) + (A.3.4), C1-2089: I.A + (H.2.2) + (A.3.4), C1-2090: I.A + (H.2.3) + (A.3.4),
C1-2091: I.A + (H.2.5) + (A.3.4), C1-2092: I.A + (H.2.7) + (A.3.4), C1-2093: I.A + (H.2.8) +
(A.3.4), C1-2094: I.A + (H.3.2) + (A.3.4), C1-2095: I.A + (H.3.4) + (A.3.4), C1-2096: I.A + (H.3.5) +
(A.3.4), C1-2097: I.A + (H.4.9) + (A.3.4), C1-2098: I.A + (H.4.10) + (A.3.4), C1-2099: I.A +
(I.2.2) + (A.3.4), C1-2100: I.A + (I.2.5) + (A.3.4), C1-2101: I.A + (J.1.2) + (A.3.4), C1-2102: I.A +
(J.1.5) + (A.3.4), C1-2103: I.A + (J.1.8) + (A.3.4), C1-2104: I.A + (J.1.11) + (A.3.4), C1-2105: I.A +
(J.1.12) + (A.3.4), C1-2106: I.A + (K.1.3) + (A.3.4), C1-2107: I.A + (K.1.29) + (A.3.4), C1-
2108: I.A + (K.1.30) + (A.3.4), C1-2109: I.A + (K.1.31) + (A.3.4), C1-2110: I.A + (K.1.32) +
(A.3.4), C1-2111: I.A + (K.1.41) + (A.3.4), C1-2112: I.A + (K.1.42) + (A.3.4), C1-2113: I.A +
(K.1.44) + (A.3.4), C1-2114: I.A + (K.1.45) + (A.3.4), C1-2115: I.A + (K.1.47) + (A.3.4), C1-2116:
I.A + (K.1.49) + (A.3.4), C1-2117: I.A + (K.1.53) + (A.3.4), C1-2118: I.A + (K.1.54) + (A.3.4), C1-
2119: I.A + (A.1.4) + (A.3.7), C1-2120: I.A + (A.1.12) + (A.3.7), C1-2121: I.A + (A.1.21) +
(A.3.7), C1-2122: I.A + (A.1,21a) + (A.3.7), C1-2123: I.A + (A.1.24) + (A.3.7), C1-2124: I.A +
(A.1.26) + (A.3.7), C1-2125: I.A + (A.1.27) + (A.3.7), C1-2126: I.A + (A.1.30) + (A.3.7), C1-2127:
I.A + (A.1.31) + (A.3.7), C1-2128: I.A + (A.1.32) + (A.3.7), C1-2129: I.A + (A.1.35) + (A.3.7), C1-
2130: I.A + (A.2.1) + (A.3.7), C1-2131: I.A + (A.2.3) + (A.3.7), C1-2132: I.A + (A.2.5) + (A.3.7),
C1-2133: I.A + (A.3.9) + (A.3.7), C1-2134: I.A + (A.3.11) + (A.3.7), C1-2135: I.A + (A.3.12) +
(A.3.7), C1-2136: I.A + (A.3.15) + (A.3.7), C1-2137: I.A + (A.3.16) + (A.3.7), C1-2138: I.A +
(A.3.17) + (A.3.7), C1-2139: I.A + (A.3.18) + (A.3.7), C1-2140: I.A + (A.3.19) + (A.3.7), C1-2141:
I.A + (A.3.20) + (A.3.7), C1-2142: I.A + (A.3.21) + (A.3.7), C1-2143: I.A + (A.3.22) + (A.3.7), C1-
2144: I.A + (A.3.23) + (A.3.7), C1-2145: I.A + (A.3.24) + (A.3.7), C1-2146: I.A + (A.3.25) +
(A.3.7), C1-2147: I.A + (A.3.26) + (A.3.7), C1-2148: I.A + (A.3.27) + (A.3.7), C1-2149: I.A +
(A.3.28) + (A.3.7), C1-2150: I.A + (A.3.29) + (A.3.7), C1-2151: I.A + (A.3.31) + (A.3.7), C1-2152:
I.A + (A.3.32) + (A.3.7), C1-2153: I.A + (A.3.33) + (A.3.7), C1-2154: I.A + (A.3.34) + (A.3.7), C1-
2155: I.A + (A.3.35) + (A.3.7), C1-2156: I.A + (A.3.36) + (A.3.7), C1-2157: I.A + (A.3.37) +
(A.3.7), C1-2158: I.A + (A.3.38) + (A.3.7), C1-2159: I.A + (A.3.39) + (A.3.7), C1-2160: I.A +
(A.4.5) + (A.3.7), C1-2161: I.A + (A.4.11) + (A.3.7), C1-2162: I.A + (B.1.4) + (A.3.7), C1-2163:
I.A + (B.1.5) + (A.3.7), C1-2164: I.A + (B.1.8.) + (A.3.7), C1-2165: I.A + (B.1.10) + (A.3.7), C1-
2166: I.A + (B.1.11) + (A.3.7), C1-2167: I.A + (B.1.12) + (A.3.7), C1-2168: I.A + (B.1.13) +
(A.3.7), C1-2169: I.A + (B.1.17) + (A.3.7), C1-2170: I.A + (B.1.18) + (A.3.7), C1-2171: I.A +
(B.1.21) + (A.3.7), C1-2172: I.A + (B.1.22) + (A.3.7), C1-2173: I.A + (B.1.23) + (A.3.7), C1-2174:
I.A + (B.1.25) + (A.3.7), C1-2175: I.A + (B.1.26) + (A.3.7), C1-2176: I.A + (B.1.29) + (A.3.7), C1-
2177: I.A + (B.1.33) + (A.3.7), C1-2178: I.A + (B.1.34) + (A.3.7), C1-2179: I.A + (B.1.37) +
(A.3.7), C1-2180: I.A + (B.1.38) + (A.3.7), C1-2181: I.A + (B.1.43) + (A.3.7), C1-2182: I.A +
(B.1.46) + (A.3.7), C1-2183: I.A + (B.2.4) + (A.3.7), C1-2184: I.A + (B.2.5) + (A.3.7), C1-2185:
I.A + (B.2.6) + (A.3.7), C1-2186: I.A + (B.2.8) + (A.3.7), C1-2187: I.A + (C.1.1) + (A.3.7), C1-
2188: I.A + (C.1.2) + (A.3.7), C1-2189: I.A + (C.1.4) + (A.3.7), C1-2190: I.A + (C.1.5) + (A.3.7),
C1-2191: I.A + (C.2.6) + (A.3.7), C1-2192: I.A + (C.2.7) + (A.3.7), C1-2193: I.A + (C.2.8) +
(A.3.7), C1-2194: I.A + (D.1.1) + (A.3.7), C1-2195: I.A + (D.1.2) + (A.3.7), C1-2196: I.A + (D.1.5) +
(A.3.7), C1-2197: I.A + (D.2.4) + (A.3.7), C1-2198: I.A + (D.2.6) + (A.3.7), C1-2199: I.A +
(D.2.7) + (A.3.7), C1-2200: I.A + (E.1.1) + (A.3.7), C1-2201: I.A + (E.1.3) + (A.3.7), C1-2202: I.A +
(E.2.2) + (A.3.7), C1-2203: I.A + (E.2.3) + (A.3.7), C1-2204: I.A + (F.1.2) + (A.3.7), C1-2205:
I.A + (F.1.4) + (A.3.7), C1-2206: I.A + (F.1.5) + (A.3.7), C1-2207: I.A + (G.3.1) + (A.3.7), C1-
2208: I.A + (G.3.3) + (A.3.7), C1-2209: I.A + (G.3.6) + (A.3.7), C1-2210: I.A + (G.5.1) + (A.3.7),
C1-2211: I.A + (G.5.2) + (A.3.7), C1-2212: I.A + (G.5.3) + (A.3.7), C1-2213: I.A + (G.5.4) +
(A.3.7), C1-2214: I.A + (G.5.5) + (A.3.7), C1-2215: I.A + (G.5.6) + (A.3.7), C1-2216: I.A +
(G.5.7) + (A.3.7), C1-2217: I.A + (G.5.8) + (A.3.7), C1-2218: I.A + (G.5.9) + (A.3.7), C1-2219:
I.A + (G.5.10) + (A.3.7), C1-2220: I.A + (G.5.11) + (A.3.7), C1-2221: I.A + (H.1.5) + (A.3.7), C1-
2222: I.A + (H.1.7) + (A.3.7), C1-2223: I.A + (H.2.2) + (A.3.7), C1-2224: I.A + (H.2.3) + (A.3.7),
C1-2225: I.A + (H.2.5) + (A.3.7), C1-2226: I.A + (H.2.7) + (A.3.7), C1-2227: I.A + (H.2.8) +
(A.3.7), C1-2228: I.A + (H.3.2) + (A.3.7), C1-2229: I.A + (H.3.4) + (A.3.7), C1-2230: I.A + (H.3.5) +
(A.3.7), C1-2231: I.A + (H.4.9) + (A.3.7), C1-2232: I.A + (H.4.10) + (A.3.7), C1-2233: I.A +
(I.2.2) + (A.3.7), C1-2234: I.A + (I.2.5) + (A.3.7), C1-2235: I.A + (J.1.2) + (A.3.7), C1-2236: I.A +
(J.1.5) + (A.3.7), C1-2237: I.A + (J.1.8) + (A.3.7), C1-2238: I.A + (J.1.11) + (A.3.7), C1-2239: I.A +
(J.1.12) + (A.3.7), C1-2240: I.A + (K.1.3) + (A.3.7), C1-2241: I.A + (K.1.29) + (A.3.7), C1-
2242: I.A + (K.1.30) + (A.3.7), C1-2243: I.A + (K.1.31) + (A.3.7), C1-2244: I.A + (K.1.32) +
(A.3.7), C1-2245: I.A + (K.1.41) + (A.3.7), C1-2246: I.A + (K.1.42) + (A.3.7), C1-2247: I.A +
(K.1.44) + (A.3.7), C1-2248: I.A + (K.1.45) + (A.3.7), C1-2249: I.A + (K.1.47) + (A.3.7), C1-2250:
I.A + (K.1.49) + (A.3.7), C1-2251: I.A + (K.1.53) + (A.3.7), C1-2252: I.A + (K.1.54) + (A.3.7), C1-
2253: I.A + (A.1.4) + (A.3.9), C1-2254: I.A + (A.1.12) + (A.3.9), C1-2255: I.A + (A.1.21) +
(A.3.9), C1-2256: I.A + (A.1,21a) + (A.3.9), C1-2257: I.A + (A.1.24) + (A.3.9), C1-2258: I.A +
(A.1.26) + (A.3.9), C1-2259: I.A + (A.1.27) + (A.3.9), C1-2260: I.A + (A.1.30) + (A.3.9), C1-2261:
I.A + (A.1.31) + (A.3.9), C1-2262: I.A + (A.1.32) + (A.3.9), C1-2263: I.A + (A.1.35) + (A.3.9), C1-
2264: I.A + (A.2.1) + (A.3.9), C1-2265: I.A + (A.2.3) + (A.3.9), C1-2266: I.A + (A.2.5) + (A.3.9),
C1-2267: I.A + (A.3.11) + (A.3.9), C1-2268: I.A + (A.3.12) + (A.3.9), C1-2269: I.A + (A.3.15) +
(A.3.9), C1-2270: I.A + (A.3.16) + (A.3.9), C1-2271: I.A + (A.3.17) + (A.3.9), C1-2272: I.A +
(A.3.18) + (A.3.9), C1-2273: I.A + (A.3.19) + (A.3.9), C1-2274: I.A + (A.3.20) + (A.3.9), C1-2275:
I.A + (A.3.21) + (A.3.9), C1-2276: I.A + (A.3.22) + (A.3.9), C1-2277: I.A + (A.3.23) + (A.3.9), C1-
2278: I.A + (A.3.24) + (A.3.9), C1-2279: I.A + (A.3.25) + (A.3.9), C1-2280: I.A + (A.3.26) +
(A.3.9), C1-2281: I.A + (A.3.27) + (A.3.9), C1-2282: I.A + (A.3.28) + (A.3.9), C1-2283: I.A +
(A.3.29) + (A.3.9), C1-2284: I.A + (A.3.31) + (A.3.9), C1-2285: I.A + (A.3.32) + (A.3.9), C1-2286:
I.A + (A.3.33) + (A.3.9), C1-2287: I.A + (A.3.34) + (A.3.9), C1-2288: I.A + (A.3.35) + (A.3.9), C1-
2289: I.A + (A.3.36) + (A.3.9), C1-2290: I.A + (A.3.37) + (A.3.9), C1-2291: I.A + (A.3.38) +
(A.3.9), C1-2292: I.A + (A.3.39) + (A.3.9), C1-2293: I.A + (A.4.5) + (A.3.9), C1-2294: I.A +
(A.4.11) + (A.3.9), C1-2295: I.A + (B.1.4) + (A.3.9), C1-2296: I.A + (B.1.5) + (A.3.9), C1-2297:
I.A + (B.1.8.) + (A.3.9), C1-2298: I.A + (B.1.10) + (A.3.9), C1-2299: I.A + (B.1.11) + (A.3.9), C1-
2300: I.A + (B.1.12) + (A.3.9), C1-2301: I.A + (B.1.13) + (A.3.9), C1-2302: I.A + (B.1.17) +
(A.3.9), C1-2303: I.A + (B.1.18) + (A.3.9), C1-2304: I.A + (B.1.21) + (A.3.9), C1-2305: I.A +
(B.1.22) + (A.3.9), C1-2306: I.A + (B.1.23) + (A.3.9), C1-2307: I.A + (B.1.25) + (A.3.9), C1-2308:
I.A + (B.1.26) + (A.3.9), C1-2309: I.A + (B.1.29) + (A.3.9), C1-2310: I.A + (B.1.33) + (A.3.9), C1-
2311: I.A + (B.1.34) + (A.3.9), C1-2312: I.A + (B.1.37) + (A.3.9), C1-2313: I.A + (B.1.38) +
(A.3.9), C1-2314: I.A + (B.1.43) + (A.3.9), C1-2315: I.A + (B.1.46) + (A.3.9), C1-2316: I.A +
(B.2.4) + (A.3.9), C1-2317: I.A + (B.2.5) + (A.3.9), C1-2318: I.A + (B.2.6) + (A.3.9), C1-2319: I.A +
(B.2.8) + (A.3.9), C1-2320: I.A + (C.1.1) + (A.3.9), C1-2321: I.A + (C.1.2) + (A.3.9), C1-2322:
I.A + (C.1.4) + (A.3.9), C1-2323: I.A + (C.1.5) + (A.3.9), C1-2324: I.A + (C.2.6) + (A.3.9), C1-
2325: I.A + (C.2.7) + (A.3.9), C1-2326: I.A + (C.2.8) + (A.3.9), C1-2327: I.A + (D.1.1) + (A.3.9),
C1-2328: I.A + (D.1.2) + (A.3.9), C1-2329: I.A + (D.1.5) + (A.3.9), C1-2330: I.A + (D.2.4) +
(A.3.9), C1-2331: I.A + (D.2.6) + (A.3.9), C1-2332: I.A + (D.2.7) + (A.3.9), C1-2333: I.A + (E.1.1) +
(A.3.9), C1-2334: I.A + (E.1.3) + (A.3.9), C1-2335: I.A + (E.2.2) + (A.3.9), C1-2336: I.A +
(E.2.3) + (A.3.9), C1-2337: I.A + (F.1.2) + (A.3.9), C1-2338: I.A + (F.1.4) + (A.3.9), C1-2339: I.A +
(F.1.5) + (A.3.9), C1-2340: I.A + (G.3.1) + (A.3.9), C1-2341: I.A + (G.3.3) + (A.3.9), C1-2342:
I.A + (G.3.6) + (A.3.9), C1-2343: I.A + (G.5.1) + (A.3.9), C1-2344: I.A + (G.5.2) + (A.3.9), C1-
2345: I.A + (G.5.3) + (A.3.9), C1-2346: I.A + (G.5.4) + (A.3.9), C1-2347: I.A + (G.5.5) + (A.3.9),
C1-2348: I.A + (G.5.6) + (A.3.9), C1-2349: I.A + (G.5.7) + (A.3.9), C1-2350: I.A + (G.5.8) +
(A.3.9), C1-2351: I.A + (G.5.9) + (A.3.9), C1-2352: I.A + (G.5.10) + (A.3.9), C1-2353: I.A +
(G.5.11) + (A.3.9), C1-2354: I.A + (H.1.5) + (A.3.9), C1-2355: I.A + (H.1.7) + (A.3.9), C1-2356:
I.A + (H.2.2) + (A.3.9), C1-2357: I.A + (H.2.3) + (A.3.9), C1-2358: I.A + (H.2.5) + (A.3.9), C1-
2359: I.A + (H.2.7) + (A.3.9), C1-2360: I.A + (H.2.8) + (A.3.9), C1-2361: I.A + (H.3.2) + (A.3.9),
C1-2362: I.A + (H.3.4) + (A.3.9), C1-2363: I.A + (H.3.5) + (A.3.9), C1-2364: I.A + (H.4.9) +
(A.3.9), C1-2365: I.A + (H.4.10) + (A.3.9), C1-2366: I.A + (I.2.2) + (A.3.9), C1-2367: I.A + (I.2.5) +
(A.3.9), C1-2368: I.A + (J.1.2) + (A.3.9), C1-2369: I.A + (J.1.5) + (A.3.9), C1-2370: I.A +
(J.1.8) + (A.3.9), C1-2371: I.A + (J.1.11) + (A.3.9), C1-2372: I.A + (J.1.12) + (A.3.9), C1-2373:
I.A + (K.1.3) + (A.3.9), C1-2374: I.A + (K.1.29) + (A.3.9), C1-2375: I.A + (K.1.30) + (A.3.9), C1-
2376: I.A + (K.1.31) + (A.3.9), C1-2377: I.A + (K.1.32) + (A.3.9), C1-2378: I.A + (K.1.41) +
(A.3.9), C1-2379: I.A + (K.1.42) + (A.3.9), C1-2380: I.A + (K.1.44) + (A.3.9), C1-2381: I.A +
(K.1.45) + (A.3.9), C1-2382: I.A + (K.1.47) + (A.3.9), C1-2383: I.A + (K.1.49) + (A.3.9), C1-2384:
I.A + (K.1.53) + (A.3.9), C1-2385: I.A + (K.1.54) + (A.3.9), C1-2386: I.A + (A.1.4) + (A.3.17), C1-
2387: I.A + (A.1.12) + (A.3.17), C1-2388: I.A + (A.1.21) + (A.3.17), C1-2389: I.A + (A.1,21a) +
(A.3.17), C1-2390: I.A + (A.1.24) + (A.3.17), C1-2391: I.A + (A.1.26) + (A.3.17), C1-2392: I.A +
(A.1.27) + (A.3.17), C1-2393: I.A + (A.1.30) + (A.3.17), C1-2394: I.A + (A.1.31) + (A.3.17), C1-
2395: I.A + (A.1.32) + (A.3.17), C1-2396: I.A + (A.1.35) + (A.3.17), C1-2397: I.A + (A.2.1) +
(A.3.17), C1-2398: I.A + (A.2.3) + (A.3.17), C1-2399: I.A + (A.2.5) + (A.3.17), C1-2400: I.A +
(A.3.11) + (A.3.17), C1-2401: I.A + (A.3.12) + (A.3.17), C1-2402: I.A + (A.3.15) + (A.3.17), C1-
2403: I.A + (A.3.16) + (A.3.17), C1-2404: I.A + (A.3.18) + (A.3.17), C1-2405: I.A + (A.3.19) +
(A.3.17), C1-2406: I.A + (A.3.20) + (A.3.17), C1-2407: I.A + (A.3.21) + (A.3.17), C1-2408: I.A +
(A.3.22) + (A.3.17), C1-2409: I.A + (A.3.23) + (A.3.17), C1-2410: I.A + (A.3.24) + (A.3.17), C1-
2411: I.A + (A.3.25) + (A.3.17), C1-2412: I.A + (A.3.26) + (A.3.17), C1-2413: I.A + (A.3.27) +
(A.3.17), C1-2414: I.A + (A.3.28) + (A.3.17), C1-2415: I.A + (A.3.29) + (A.3.17), C1-2416: I.A +
(A.3.31) + (A.3.17), C1-2417: I.A + (A.3.32) + (A.3.17), C1-2418: I.A + (A.3.33) + (A.3.17), C1-
2419: I.A + (A.3.34) + (A.3.17), C1-2420: I.A + (A.3.35) + (A.3.17), C1-2421: I.A + (A.3.36) +
(A.3.17), C1-2422: I.A + (A.3.37) + (A.3.17), C1-2423: I.A + (A.3.38) + (A.3.17), C1-2424: I.A +
(A.3.39) + (A.3.17), C1-2425: I.A + (A.4.5) + (A.3.17), C1-2426: I.A + (A.4.11) + (A.3.17), C1-
2427: I.A + (B.1.4) + (A.3.17), C1-2428: I.A + (B.1.5) + (A.3.17), C1-2429: I.A + (B.1.8.) +
(A.3.17), C1-2430: I.A + (B.1.10) + (A.3.17), C1-2431: I.A + (B.1.11) + (A.3.17), C1-2432: I.A +
(B.1.12) + (A.3.17), C1-2433: I.A + (B.1.13) + (A.3.17), C1-2434: I.A + (B.1.17) + (A.3.17), C1-
2435: I.A + (B.1.18) + (A.3.17), C1-2436: I.A + (B.1.21) + (A.3.17), C1-2437: I.A + (B.1.22) +
(A.3.17), C1-2438: I.A + (B.1.23) + (A.3.17), C1-2439: I.A + (B.1.25) + (A.3.17), C1-2440: I.A +
(B.1.26) + (A.3.17), C1-2441: I.A + (B.1.29) + (A.3.17), C1-2442: I.A + (B.1.33) + (A.3.17), C1-
2443: I.A + (B.1.34) + (A.3.17), C1-2444: I.A + (B.1.37) + (A.3.17), C1-2445: I.A + (B.1.38) +
(A.3.17), C1-2446: I.A + (B.1.43) + (A.3.17), C1-2447: I.A + (B.1.46) + (A.3.17), C1-2448: I.A +
(B.2.4) + (A.3.17), C1-2449: I.A + (B.2.5) + (A.3.17), C1-2450: I.A + (B.2.6) + (A.3.17), C1-2451:
I.A + (B.2.8) + (A.3.17), C1-2452: I.A + (C.1.1) + (A.3.17), C1-2453: I.A + (C.1.2) + (A.3.17), C1-
2454: I.A + (C.1.4) + (A.3.17), C1-2455: I.A + (C.1.5) + (A.3.17), C1-2456: I.A + (C.2.6) +
(A.3.17), C1-2457: I.A + (C.2.7) + (A.3.17), C1-2458: I.A + (C.2.8) + (A.3.17), C1-2459: I.A +
(D.1.1) + (A.3.17), C1-2460: I.A + (D.1.2) + (A.3.17), C1-2461: I.A + (D.1.5) + (A.3.17), C1-
2462: I.A + (D.2.4) + (A.3.17), C1-2463: I.A + (D.2.6) + (A.3.17), C1-2464: I.A + (D.2.7) +
(A.3.17), C1-2465: I.A + (E.1.1) + (A.3.17), C1-2466: I.A + (E.1.3) + (A.3.17), C1-2467: I.A +
(E.2.2) + (A.3.17), C1-2468: I.A + (E.2.3) + (A.3.17), C1-2469: I.A + (F.1.2) + (A.3.17), C1-2470:
I.A + (F.1.4) + (A.3.17), C1-2471: I.A + (F.1.5) + (A.3.17), C1-2472: I.A + (G.3.1) + (A.3.17), C1-
2473: I.A + (G.3.3) + (A.3.17), C1-2474: I.A + (G.3.6) + (A.3.17), C1-2475: I.A + (G.5.1) +
(A.3.17), C1-2476: I.A + (G.5.2) + (A.3.17), C1-2477: I.A + (G.5.3) + (A.3.17), C1-2478: I.A +
(G.5.4) + (A.3.17), C1-2479: I.A + (G.5.5) + (A.3.17), C1-2480: I.A + (G.5.6) + (A.3.17), C1-
2481: I.A + (G.5.7) + (A.3.17), C1-2482: I.A + (G.5.8) + (A.3.17), C1-2483: I.A + (G.5.9) +
(A.3.17), C1-2484: I.A + (G.5.10) + (A.3.17), C1-2485: I.A + (G.5.11) + (A.3.17), C1-2486: I.A +
(H.1.5) + (A.3.17), C1-2487: I.A + (H.1.7) + (A.3.17), C1-2488: I.A + (H.2.2) + (A.3.17), C1-
2489: I.A + (H.2.3) + (A.3.17), C1-2490: I.A + (H.2.5) + (A.3.17), C1-2491: I.A + (H.2.7) +
(A.3.17), C1-2492: I.A + (H.2.8) + (A.3.17), C1-2493: I.A + (H.3.2) + (A.3.17), C1-2494: I.A +
(H.3.4) + (A.3.17), C1-2495: I.A + (H.3.5) + (A.3.17), C1-2496: I.A + (H.4.9) + (A.3.17), C1-
2497: I.A + (H.4.10) + (A.3.17), C1-2498: I.A + (I.2.2) + (A.3.17), C1-2499: I.A + (I.2.5) +
(A.3.17), C1-2500: I.A + (J.1.2) + (A.3.17), C1-2501: I.A + (J.1.5) + (A.3.17), C1-2502: I.A +
(J.1.8) + (A.3.17), C1-2503: I.A + (J.1.11) + (A.3.17), C1-2504: I.A + (J.1.12) + (A.3.17), C1-
2505: I.A + (K.1.3) + (A.3.17), C1-2506: I.A + (K.1.29) + (A.3.17), C1-2507: I.A + (K.1.30) +
(A.3.17), C1-2508: I.A + (K.1.31) + (A.3.17), C1-2509: I.A + (K.1.32) + (A.3.17), C1-2510: I.A +
(K.1.41) + (A.3.17), C1-2511: I.A + (K.1.42) + (A.3.17), C1-2512: I.A + (K.1.44) + (A.3.17), C1-
2513: I.A + (K.1.45) + (A.3.17), C1-2514: I.A + (K.1.47) + (A.3.17), C1-2515: I.A + (K.1.49) +
(A.3.17), C1-2516: I.A + (K.1.53) + (A.3.17), C1-2517: I.A + (K.1.54) + (A.3.17), C1-2518: I.A +
(A.1.4) + (A.3.24), C1-2519: I.A + (A.1.12) + (A.3.24), C1-2520: I.A + (A.1.21) + (A.3.24), C1-
2521: I.A + (A.1.21a) + (A.3.24), C1-2522: I.A + (A.1.24) + (A.3.24), C1-2523: I.A + (A.1.26) +
(A.3.24), C1-2524: I.A + (A.1.27) + (A.3.24), C1-2525: I.A + (A.1.30) + (A.3.24), C1-2526: I.A +
(A.1.31) + (A.3.24), C1-2527: I.A + (A.1.32) + (A.3.24), C1-2528: I.A + (A.1.35) + (A.3.24), C1-
2529: I.A + (A.2.1) + (A.3.24), C1-2530: I.A + (A.2.3) + (A.3.24), C1-2531: I.A + (A.2.5) +
(A.3.24), C1-2532: I.A + (A.3.11) + (A.3.24), C1-2533: I.A + (A.3.12) + (A.3.24), C1-2534: I.A +
(A.3.15) + (A.3.24), C1-2535: I.A + (A.3.16) + (A.3.24), C1-2536: I.A + (A.3.18) + (A.3.24), C1-
2537: I.A + (A.3.19) + (A.3.24), C1-2538: I.A + (A.3.20) + (A.3.24), C1-2539: I.A + (A.3.21) +
(A.3.24), C1-2540: I.A + (A.3.22) + (A.3.24), C1-2541: I.A + (A.3.23) + (A.3.24), C1-2542: I.A +
(A.3.25) + (A.3.24), C1-2543: I.A + (A.3.26) + (A.3.24), C1-2544: I.A + (A.3.27) + (A.3.24), C1-
2545: I.A + (A.3.28) + (A.3.24), C1-2546: I.A + (A.3.29) + (A.3.24), C1-2547: I.A + (A.3.31) +
(A.3.24), C1-2548: I.A + (A.3.32) + (A.3.24), C1-2549: I.A + (A.3.33) + (A.3.24), C1-2550: I.A +
(A.3.34) + (A.3.24), C1-2551: I.A + (A.3.35) + (A.3.24), C1-2552: I.A + (A.3.36) + (A.3.24), C1-
2553: I.A + (A.3.37) + (A.3.24), C1-2554: I.A + (A.3.38) + (A.3.24), C1-2555: I.A + (A.3.39) +
(A.3.24), C1-2556: I.A + (A.4.5) + (A.3.24), C1-2557: I.A + (A.4.11) + (A.3.24), C1-2558: I.A +
(B.1.4) + (A.3.24), C1-2559: I.A + (B.1.5) + (A.3.24), C1-2560: I.A + (B.1.8.) + (A.3.24), C1-
2561: I.A + (B.1.10) + (A.3.24), C1-2562: I.A + (B.1.11) + (A.3.24), C1-2563: I.A + (B.1.12) +
(A.3.24), C1-2564: I.A + (B.1.13) + (A.3.24), C1-2565: I.A + (B.1.17) + (A.3.24), C1-2566: I.A +
(B.1.18) + (A.3.24), C1-2567: I.A + (B.1.21) + (A.3.24), C1-2568: I.A + (B.1.22) + (A.3.24), C1-
2569: I.A + (B.1.23) + (A.3.24), C1-2570: I.A + (B.1.25) + (A.3.24), C1-2571: I.A + (B.1.26) +
(A.3.24), C1-2572: I.A + (B.1.29) + (A.3.24), C1-2573: I.A + (B.1.33) + (A.3.24), C1-2574: I.A +
(B.1.34) + (A.3.24), C1-2575: I.A + (B.1.37) + (A.3.24), C1-2576: I.A + (B.1.38) + (A.3.24), C1-
2577: I.A + (B.1.43) + (A.3.24), C1-2578: I.A + (B.1.46) + (A.3.24), C1-2579: I.A + (B.2.4) +
(A.3.24), C1-2580: I.A + (B.2.5) + (A.3.24), C1-2581: I.A + (B.2.6) + (A.3.24), C1-2582: I.A +
(B.2.8) + (A.3.24), C1-2583: I.A + (C.1.1) + (A.3.24), C1-2584: I.A + (C.1.2) + (A.3.24), C1-2585:
I.A + (C.1.4) + (A.3.24), C1-2586: I.A + (C.1.5) + (A.3.24), C1-2587: I.A + (C.2.6) + (A.3.24), C1-
2588: I.A + (C.2.7) + (A.3.24), C1-2589: I.A + (C.2.8) + (A.3.24), C1-2590: I.A + (D.1.1) +
(A.3.24), C1-2591: I.A + (D.1.2) + (A.3.24), C1-2592: I.A + (D.1.5) + (A.3.24), C1-2593: I.A +
(D.2.4) + (A.3.24), C1-2594: I.A + (D.2.6) + (A.3.24), C1-2595: I.A + (D.2.7) + (A.3.24), C1-
2596: I.A + (E.1.1) + (A.3.24), C1-2597: I.A + (E.1.3) + (A.3.24), C1-2598: I.A + (E.2.2) +
(A.3.24), C1-2599: I.A + (E.2.3) + (A.3.24), C1-2600: I.A + (F.1.2) + (A.3.24), C1-2601: I.A +
(F.1.4) + (A.3.24), C1-2602: I.A + (F.1.5) + (A.3.24), C1-2603: I.A + (G.3.1) + (A.3.24), C1-2604:
I.A + (G.3.3) + (A.3.24), C1-2605: I.A + (G.3.6) + (A.3.24), C1-2606: I.A + (G.5.1) + (A.3.24),
C1-2607: I.A + (G.5.2) + (A.3.24), C1-2608: I.A + (G.5.3) + (A.3.24), C1-2609: I.A + (G.5.4) +
(A.3.24), C1-2610: I.A + (G.5.5) + (A.3.24), C1-2611: I.A + (G.5.6) + (A.3.24), C1-2612: I.A +
(G.5.7) + (A.3.24), C1-2613: I.A + (G.5.8) + (A.3.24), C1-2614: I.A + (G.5.9) + (A.3.24), C1-
2615: I.A + (G.5.10) + (A.3.24), C1-2616: I.A + (G.5.11) + (A.3.24), C1-2617: I.A + (H.1.5) +
(A.3.24), C1-2618: I.A + (H.1.7) + (A.3.24), C1-2619: I.A + (H.2.2) + (A.3.24), C1-2620: I.A +
(H.2.3) + (A.3.24), C1-2621: I.A + (H.2.5) + (A.3.24), C1-2622: I.A + (H.2.7) + (A.3.24), C1-
2623: I.A + (H.2.8) + (A.3.24), C1-2624: I.A + (H.3.2) + (A.3.24), C1-2625: I.A + (H.3.4) +
(A.3.24), C1-2626: I.A + (H.3.5) + (A.3.24), C1-2627: I.A + (H.4.9) + (A.3.24), C1-2628: I.A +
(H.4.10) + (A.3.24), C1-2629: I.A + (I.2.2) + (A.3.24), C1-2630: I.A + (I.2.5) + (A.3.24), C1-2631:
I.A + (J.1.2) + (A.3.24), C1-2632: I.A + (J.1.5) + (A.3.24), C1-2633: I.A + (J.1.8) + (A.3.24), C1-
2634: I.A + (J.1.11) + (A.3.24), C1-2635: I.A + (J.1.12) + (A.3.24), C1-2636: I.A + (K.1.3) +
(A.3.24), C1-2637: I.A + (K.1.29) + (A.3.24), C1-2638: I.A + (K.1.30) + (A.3.24), C1-2639: I.A +
(K.1.31) + (A.3.24), C1-2640: I.A + (K.1.32) + (A.3.24), C1-2641: I.A + (K.1.41) + (A.3.24), C1-
2642: I.A + (K.1.42) + (A.3.24), C1-2643: I.A + (K.1.44) + (A.3.24), C1-2644: I.A + (K.1.45) +
(A.3.24), C1-2645: I.A + (K.1.47) + (A.3.24), C1-2646: I.A + (K.1.49) + (A.3.24), C1-2647: I.A +
(K.1.53) + (A.3.24), C1-2648: I.A + (K.1.54) + (A.3.24), C1-2649: I.A + (A.1.4) + (A.3.28), C1-
2650: I.A + (A.1.12) + (A.3.28), C1-2651: I.A + (A.1.21) + (A.3.28), C1-2652: I.A + (A.1,21a) +
(A.3.28), C1-2653: I.A + (A.1.24) + (A.3.28), C1-2654: I.A + (A.1.26) + (A.3.28), C1-2655: I.A +
(A.1.27) + (A.3.28), C1-2656: I.A + (A.1.30) + (A.3.28), C1-2657: I.A + (A.1.31) + (A.3.28), C1-
2658: I.A + (A.1.32) + (A.3.28), C1-2659: I.A + (A.1.35) + (A.3.28), C1-2660: I.A + (A.2.1) +
(A.3.28), C1-2661: I.A + (A.2.3) + (A.3.28), C1-2662: I.A + (A.2.5) + (A.3.28), C1-2663: I.A +
(A.3.11) + (A.3.28), C1-2664: I.A + (A.3.12) + (A.3.28), C1-2665: I.A + (A.3.15) + (A.3.28), C1-
2666: I.A + (A.3.16) + (A.3.28), C1-2667: I.A + (A.3.18) + (A.3.28), C1-2668: I.A + (A.3.19) +
(A.3.28), C1-2669: I.A + (A.3.20) + (A.3.28), C1-2670: I.A + (A.3.21) + (A.3.28), C1-2671: I.A +
(A.3.22) + (A.3.28), C1-2672: I.A + (A.3.23) + (A.3.28), C1-2673: I.A + (A.3.25) + (A.3.28), C1-
2674: I.A + (A.3.26) + (A.3.28), C1-2675: I.A + (A.3.27) + (A.3.28), C1-2676: I.A + (A.3.29) +
(A.3.28), C1-2677: I.A + (A.3.31) + (A.3.28), C1-2678: I.A + (A.3.32) + (A.3.28), C1-2679: I.A +
(A.3.33) + (A.3.28), C1-2680: I.A + (A.3.34) + (A.3.28), C1-2681: I.A + (A.3.35) + (A.3.28), C1-
2682: I.A + (A.3.36) + (A.3.28), C1-2683: I.A + (A.3.37) + (A.3.28), C1-2684: I.A + (A.3.38) +
(A.3.28), C1-2685: I.A + (A.3.39) + (A.3.28), C1-2686: I.A + (A.4.5) + (A.3.28), C1-2687: I.A +
(A.4.11) + (A.3.28), C1-2688: I.A + (B.1.4) + (A.3.28), C1-2689: I.A + (B.1.5) + (A.3.28), C1-
2690: I.A + (B.1.8.) + (A.3.28), C1-2691: I.A + (B.1.10) + (A.3.28), C1-2692: I.A + (B.1.11) +
(A.3.28), C1-2693: I.A + (B.1.12) + (A.3.28), C1-2694: I.A + (B.1.13) + (A.3.28), C1-2695: I.A +
(B.1.17) + (A.3.28), C1-2696: I.A + (B.1.18) + (A.3.28), C1-2697: I.A + (B.1.21) + (A.3.28), C1-
2698: I.A + (B.1.22) + (A.3.28), C1-2699: I.A + (B.1.23) + (A.3.28), C1-2700: I.A + (B.1.25) +
(A.3.28), C1-2701: I.A + (B.1.26) + (A.3.28), C1-2702: I.A + (B.1.29) + (A.3.28), C1-2703: I.A +
(B.1.33) + (A.3.28), C1-2704: I.A + (B.1.34) + (A.3.28), C1-2705: I.A + (B.1.37) + (A.3.28), C1-
2706: I.A + (B.1.38) + (A.3.28), C1-2707: I.A + (B.1.43) + (A.3.28), C1-2708: I.A + (B.1.46) +
(A.3.28), C1-2709: I.A + (B.2.4) + (A.3.28), C1-2710: I.A + (B.2.5) + (A.3.28), C1-2711: I.A +
(B.2.6) + (A.3.28), C1-2712: I.A + (B.2.8) + (A.3.28), C1-2713: I.A + (C.1.1) + (A.3.28), C1-2714:
I.A + (C.1.2) + (A.3.28), C1-2715: I.A + (C.1.4) + (A.3.28), C1-2716: I.A + (C.1.5) + (A.3.28), C1-
2717: I.A + (C.2.6) + (A.3.28), C1-2718: I.A + (C.2.7) + (A.3.28), C1-2719: I.A + (C.2.8) +
(A.3.28), C1-2720: I.A + (D.1.1) + (A.3.28), C1-2721: I.A + (D.1.2) + (A.3.28), C1-2722: I.A +
(D.1.5) + (A.3.28), C1-2723: I.A + (D.2.4) + (A.3.28), C1-2724: I.A + (D.2.6) + (A.3.28), C1-
2725: I.A + (D.2.7) + (A.3.28), C1-2726: I.A + (E.1.1) + (A.3.28), C1-2727: I.A + (E. 1.3) +
(A.3.28), C1-2728: I.A + (E.2.2) + (A.3.28), C1-2729: I.A + (E.2.3) + (A.3.28), C1-2730: I.A +
(F.1.2) + (A.3.28), C1-2731: I.A + (F. 1.4) + (A.3.28), C1-2732: I.A + (F. 1.5) + (A.3.28), C1-2733:
I.A + (G.3.1) + (A.3.28), C1-2734: I.A + (G.3.3) + (A.3.28), C1-2735: I.A + (G.3.6) + (A.3.28),
C1-2736: I.A + (G.5.1) + (A.3.28), C1-2737: I.A + (G.5.2) + (A.3.28), C1-2738: I.A + (G.5.3) +
(A.3.28), C1-2739: I.A + (G.5.4) + (A.3.28), C1-2740: I.A + (G.5.5) + (A.3.28), C1-2741: I.A +
(G.5.6) + (A.3.28), C1-2742: I.A + (G.5.7) + (A.3.28), C1-2743: I.A + (G.5.8) + (A.3.28), C1-
2744: I.A + (G.5.9) + (A.3.28), C1-2745: I.A + (G.5.10) + (A.3.28), C1-2746: I.A + (G.5.11) +
(A.3.28), C1-2747: I.A + (H.1.5) + (A.3.28), C1-2748: I.A + (H.1.7) + (A.3.28), C1-2749: I.A +
(H.2.2) + (A.3.28), C1-2750: I.A + (H.2.3) + (A.3.28), C1-2751: I.A + (H.2.5) + (A.3.28), C1-
2752: I.A + (H.2.7) + (A.3.28), C1-2753: I.A + (H.2.8) + (A.3.28), C1-2754: I.A + (H.3.2) +
(A.3.28), C1-2755: I.A + (H.3.4) + (A.3.28), C1-2756: I.A + (H.3.5) + (A.3.28), C1-2757: I.A +
(H.4.9) + (A.3.28), C1-2758: I.A + (H.4.10) + (A.3.28), C1-2759: I.A + (I.2.2) + (A.3.28), C1-
2760: I.A + (I.2.5) + (A.3.28), C1-2761: I.A + (J. 1.2) + (A.3.28), C1-2762: I.A + (J. 1.5) + (A.3.28),
C1-2763: I.A + (J.1.8) + (A.3.28), C1-2764: I.A + (J.1.11) + (A.3.28), C1-2765: I.A + (J.1.12) +
(A.3.28), C1-2766: I.A + (K.1.3) + (A.3.28), C1-2767: I.A + (K.1.29) + (A.3.28), C1-2768: I.A +
(K.1.30) + (A.3.28), C1-2769: I.A + (K.1.31) + (A.3.28), C1-2770: I.A + (K.1.32) + (A.3.28), C1-
2771: I.A + (K.1.41) + (A.3.28), C1-2772: I.A + (K.1.42) + (A.3.28), C1-2773: I.A + (K.1.44) +
(A.3.28), C1-2774: I.A + (K.1.45) + (A.3.28), C1-2775: I.A + (K.1.47) + (A.3.28), C1-2776: I.A +
(K.1.49) + (A.3.28), C1-2777: I.A + (K.1.53) + (A.3.28), C1-2778: I.A + (K.1.54) + (A.3.28), C1-
2779: I.A + (A.1.4) + (A.3.29), C1-2780: I.A + (A.1.12) + (A.3.29), C1-2781: I.A + (A.1.21) +
(A.3.29), C1-2782: I.A + (A.1,21a) + (A.3.29), C1-2783: I.A + (A.1.24) + (A.3.29), C1-2784: I.A +
(A.1.26) + (A.3.29), C1-2785: I.A + (A.1.27) + (A.3.29), C1-2786: I.A + (A.1.30) + (A.3.29), C1-
2787: I.A + (A.1.31) + (A.3.29), C1-2788: I.A + (A.1.32) + (A.3.29), C1-2789: I.A + (A.1.35) +
(A.3.29), C1-2790: I.A + (A.2.1) + (A.3.29), C1-2791: I.A + (A.2.3) + (A.3.29), C1-2792: I.A +
(A.2.5) + (A.3.29), C1-2793: I.A + (A.3.11) + (A.3.29), C1-2794: I.A + (A.3.12) + (A.3.29), C1-
2795: I.A + (A.3.15) + (A.3.29), C1-2796: I.A + (A.3.16) + (A.3.29), C1-2797: I.A + (A.3.18) +
(A.3.29), C1-2798: I.A + (A.3.19) + (A.3.29), C1-2799: I.A + (A.3.20) + (A.3.29), C1-2800: I.A +
(A.3.21) + (A.3.29), C1-2801: I.A + (A.3.22) + (A.3.29), C1-2802: I.A + (A.3.23) + (A.3.29), C1-
2803: I.A + (A.3.25) + (A.3.29), C1-2804: I.A + (A.3.26) + (A.3.29), C1-2805: I.A + (A.3.27) +
(A.3.29), C1-2806: I.A + (A.3.31) + (A.3.29), C1-2807: I.A + (A.3.32) + (A.3.29), C1-2808: I.A +
(A.3.33) + (A.3.29), C1-2809: I.A + (A.3.34) + (A.3.29), C1-2810: I.A + (A.3.35) + (A.3.29), C1-
2811: I.A + (A.3.36) + (A.3.29), C1-2812: I.A + (A.3.37) + (A.3.29), C1-2813: I.A + (A.3.38) +
(A.3.29), C1-2814: I.A + (A.3.39) + (A.3.29), C1-2815: I.A + (A.4.5) + (A.3.29), C1-2816: I.A +
(A.4.11) + (A.3.29), C1-2817: I.A + (B.1.4) + (A.3.29), C1-2818: I.A + (B.1.5) + (A.3.29), C1-
2819: I.A + (B.1.8.) + (A.3.29), C1-2820: I.A + (B.1.10) + (A.3.29), C1-2821: I.A + (B.1.11) +
(A.3.29), C1-2822: I.A + (B.1.12) + (A.3.29), C1-2823: I.A + (B.1.13) + (A.3.29), C1-2824: I.A +
(B.1.17) + (A.3.29), C1-2825: I.A + (B.1.18) + (A.3.29), C1-2826: I.A + (B.1.21) + (A.3.29), C1-
2827: I.A + (B.1.22) + (A.3.29), C1-2828: I.A + (B.1.23) + (A.3.29), C1-2829: I.A + (B.1.25) +
(A.3.29), C1-2830: I.A + (B.1.26) + (A.3.29), C1-2831: I.A + (B.1.29) + (A.3.29), C1-2832: I.A +
(B.1.33) + (A.3.29), C1-2833: I.A + (B.1.34) + (A.3.29), C1-2834: I.A + (B.1.37) + (A.3.29), C1-
2835: I.A + (B.1.38) + (A.3.29), C1-2836: I.A + (B.1.43) + (A.3.29), C1-2837: I.A + (B.1.46) +
(A.3.29), C1-2838: I.A + (B.2.4) + (A.3.29), C1-2839: I.A + (B.2.5) + (A.3.29), C1-2840: I.A +
(B.2.6) + (A.3.29), C1-2841: I.A + (B.2.8) + (A.3.29), C1-2842: I.A + (C.1.1) + (A.3.29), C1-2843:
I.A + (C.1.2) + (A.3.29), C1-2844: I.A + (C.1.4) + (A.3.29), C1-2845: I.A + (C.1.5) + (A.3.29), C1-
2846: I.A + (C.2.6) + (A.3.29), C1-2847: I.A + (C.2.7) + (A.3.29), C1-2848: I.A + (C.2.8) +
(A.3.29), C1-2849: I.A + (D.1.1) + (A.3.29), C1-2850: I.A + (D.1.2) + (A.3.29), C1-2851: I.A +
(D.1.5) + (A.3.29), C1-2852: I.A + (D.2.4) + (A.3.29), C1-2853: I.A + (D.2.6) + (A.3.29), C1-
2854: I.A + (D.2.7) + (A.3.29), C1-2855: I.A + (E.1.1) + (A.3.29), C1-2856: I.A + (E.1.3) +
(A.3.29), C1-2857: I.A + (E.2.2) + (A.3.29), C1-2858: I.A + (E.2.3) + (A.3.29), C1-2859: I.A +
(F.1.2) + (A.3.29), C1-2860: I.A + (F.1.4) + (A.3.29), C1-2861: I.A + (F.1.5) + (A.3.29), C1-2862:
I.A + (G.3.1) + (A.3.29), C1-2863: I.A + (G.3.3) + (A.3.29), C1-2864: I.A + (G.3.6) + (A.3.29),
C1-2865: I.A + (G.5.1) + (A.3.29), C1-2866: I.A + (G.5.2) + (A.3.29), C1-2867: I.A + (G.5.3) +
(A.3.29), C1-2868: I.A + (G.5.4) + (A.3.29), C1-2869: I.A + (G.5.5) + (A.3.29), C1-2870: I.A +
(G.5.6) + (A.3.29), C1-2871: I.A + (G.5.7) + (A.3.29), C1-2872: I.A + (G.5.8) + (A.3.29), C1-
2873: I.A + (G.5.9) + (A.3.29), C1-2874: I.A + (G.5.10) + (A.3.29), C1-2875: I.A + (G.5.11) +
(A.3.29), C1-2876: I.A + (H.1.5) + (A.3.29), C1-2877: I.A + (H.1.7) + (A.3.29), C1-2878: I.A +
(H.2.2) + (A.3.29), C1-2879: I.A + (H.2.3) + (A.3.29), C1-2880: I.A + (H.2.5) + (A.3.29), C1-
2881: I.A + (H.2.7) + (A.3.29), C1-2882: I.A + (H.2.8) + (A.3.29), C1-2883: I.A + (H.3.2) +
(A.3.29), C1-2884: I.A + (H.3.4) + (A.3.29), C1-2885: I.A + (H.3.5) + (A.3.29), C1-2886: I.A +
(H.4.9) + (A.3.29), C1-2887: I.A + (H.4.10) + (A.3.29), C1-2888: I.A + (I.2.2) + (A.3.29), C1-
2889: I.A + (I.2.5) + (A.3.29), C1-2890: I.A + (J. 1.2) + (A.3.29), C1-2891: I.A + (J. 1.5) + (A.3.29),
C1-2892: I.A + (J.1.8) + (A.3.29), C1-2893: I.A + (J.1.11) + (A.3.29), C1-2894: I.A + (J.1.12) +
(A.3.29), C1-2895: I.A + (K.1.3) + (A.3.29), C1-2896: I.A + (K.1.29) + (A.3.29), C1-2897: I.A +
(K.1.30) + (A.3.29), C1-2898: I.A + (K.1.31) + (A.3.29), C1-2899: I.A + (K.1.32) + (A.3.29), C1-
2900: I.A + (K.1.41) + (A.3.29), C1-2901: I.A + (K.1.42) + (A.3.29), C1-2902: I.A + (K.1.44) +
(A.3.29), C1-2903: I.A + (K.1.45) + (A.3.29), C1-2904: I.A + (K.1.47) + (A.3.29), C1-2905: I.A +
(K.1.49) + (A.3.29), C1-2906: I.A + (K.1.53) + (A.3.29), C1-2907: I.A + (K.1.54) + (A.3.29), C1-
2908: I.A + (A.1.4) + (A.3.31), C1-2909: I.A + (A.1.12) + (A.3.31), C1-2910: I.A + (A.1.21) +
(A.3.31), C1-2911: I.A + (A.1,21a) + (A.3.31), C1-2912: I.A + (A.1.24) + (A.3.31), C1-2913: I.A +
(A.1.26) + (A.3.31), C1-2914: I.A + (A.1.27) + (A.3.31), C1-2915: I.A + (A.1.30) + (A.3.31), C1-
2916: I.A + (A.1.31) + (A.3.31), C1-2917: I.A + (A.1.32) + (A.3.31), C1-2918: I.A + (A.1.35) +
(A.3.31), C1-2919: I.A + (A.2.1) + (A.3.31), C1-2920: I.A + (A.2.3) + (A.3.31), C1-2921: I.A +
(A.2.5) + (A.3.31), C1-2922: I.A + (A.3.11) + (A.3.31), C1-2923: I.A + (A.3.12) + (A.3.31), C1-
2924: I.A + (A.3.15) + (A.3.31), C1-2925: I.A + (A.3.16) + (A.3.31), C1-2926: I.A + (A.3.18) +
(A.3.31), C1-2927: I.A + (A.3.19) + (A.3.31), C1-2928: I.A + (A.3.20) + (A.3.31), C1-2929: I.A +
(A.3.21) + (A.3.31), C1-2930: I.A + (A.3.22) + (A.3.31), C1-2931: I.A + (A.3.23) + (A.3.31), C1-
2932: I.A + (A.3.25) + (A.3.31), C1-2933: I.A + (A.3.26) + (A.3.31), C1-2934: I.A + (A.3.27) +
(A.3.31), C1-2935: I.A + (A.3.32) + (A.3.31), C1-2936: I.A + (A.3.33) + (A.3.31), C1-2937: I.A +
(A.3.34) + (A.3.31), C1-2938: I.A + (A.3.35) + (A.3.31), C1-2939: I.A + (A.3.36) + (A.3.31), C1-
2940: I.A + (A.3.37) + (A.3.31), C1-2941: I.A + (A.3.38) + (A.3.31), C1-2942: I.A + (A.3.39) +
(A.3.31), C1-2943: I.A + (A.4.5) + (A.3.31), C1-2944: I.A + (A.4.11) + (A.3.31), C1-2945: I.A +
(B.1.4) + (A.3.31), C1-2946: I.A + (B.1.5) + (A.3.31), C1-2947: I.A + (B.1.8.) + (A.3.31), C1-
2948: I.A + (B.1.10) + (A.3.31), C1-2949: I.A + (B.1.11) + (A.3.31), C1-2950: I.A + (B.1.12) +
(A.3.31), C1-2951: I.A + (B.1.13) + (A.3.31), C1-2952: I.A + (B.1.17) + (A.3.31), C1-2953: I.A +
(B.1.18) + (A.3.31), C1-2954: I.A + (B.1.21) + (A.3.31), C1-2955: I.A + (B.1.22) + (A.3.31), C1-
2956: I.A + (B.1.23) + (A.3.31), C1-2957: I.A + (B.1.25) + (A.3.31), C1-2958: I.A + (B.1.26) +
(A.3.31), C1-2959: I.A + (B.1.29) + (A.3.31), C1-2960: I.A + (B.1.33) + (A.3.31), C1-2961: I.A +
(B.1.34) + (A.3.31), C1-2962: I.A + (B.1.37) + (A.3.31), C1-2963: I.A + (B.1.38) + (A.3.31), C1-
2964: I.A + (B.1.43) + (A.3.31), C1-2965: I.A + (B.1.46) + (A.3.31), C1-2966: I.A + (B.2.4) +
(A.3.31), C1-2967: I.A + (B.2.5) + (A.3.31), C1-2968: I.A + (B.2.6) + (A.3.31), C1-2969: I.A +
(B.2.8) + (A.3.31), C1-2970: I.A + (C.1.1) + (A.3.31), C1-2971: I.A + (C.1.2) + (A.3.31), C1-2972:
I.A + (C.1.4) + (A.3.31), C1-2973: I.A + (C.1.5) + (A.3.31), C1-2974: I.A + (C.2.6) + (A.3.31), C1-
2975: I.A + (C.2.7) + (A.3.31), C1-2976: I.A + (C.2.8) + (A.3.31), C1-2977: I.A + (D.1.1) +
(A.3.31), C1-2978: I.A + (D.1.2) + (A.3.31), C1-2979: I.A + (D.1.5) + (A.3.31), C1-2980: I.A +
(D.2.4) + (A.3.31), C1-2981: I.A + (D.2.6) + (A.3.31), C1-2982: I.A + (D.2.7) + (A.3.31), C1-
2983: I.A + (E.1.1) + (A.3.31), C1-2984: I.A + (E.1.3) + (A.3.31), C1-2985: I.A + (E.2.2) +
(A.3.31), C1-2986: I.A + (E.2.3) + (A.3.31), C1-2987: I.A + (F.1.2) + (A.3.31), C1-2988: I.A +
(F.1.4) + (A.3.31), C1-2989: I.A + (F.1.5) + (A.3.31), C1-2990: I.A + (G.3.1) + (A.3.31), C1-2991:
I.A + (G.3.3) + (A.3.31), C1-2992: I.A + (G.3.6) + (A.3.31), C1-2993: I.A + (G.5.1) + (A.3.31),
C1-2994: I.A + (G.5.2) + (A.3.31), C1-2995: I.A + (G.5.3) + (A.3.31), C1-2996: I.A + (G.5.4) +
(A.3.31), C1-2997: I.A + (G.5.5) + (A.3.31), C1-2998: I.A + (G.5.6) + (A.3.31), C1-2999: I.A +
(G.5.7) + (A.3.31), C1-3000: I.A + (G.5.8) + (A.3.31), C1-3001: I.A + (G.5.9) + (A.3.31), C1-
3002: I.A + (G.5.10) + (A.3.31), C1-3003: I.A + (G.5.11) + (A.3.31), C1-3004: I.A + (H.1.5) +
(A.3.31), C1-3005: I.A + (H.1.7) + (A.3.31), C1-3006: I.A + (H.2.2) + (A.3.31), C1-3007: I.A +
(H.2.3) + (A.3.31), C1-3008: I.A + (H.2.5) + (A.3.31), C1-3009: I.A + (H.2.7) + (A.3.31), C1-
3010: I.A + (H.2.8) + (A.3.31), C1-3011: I.A + (H.3.2) + (A.3.31), C1-3012: I.A + (H.3.4) +
(A.3.31), C1-3013: I.A + (H.3.5) + (A.3.31), C1-3014: I.A + (H.4.9) + (A.3.31), C1-3015: I.A +
(H.4.10) + (A.3.31), C1-3016: I.A + (I.2.2) + (A.3.31), C1-3017: I.A + (I.2.5) + (A.3.31), C1-3018:
I.A + (J.1.2) + (A.3.31), C1-3019: I.A + (J.1.5) + (A.3.31), C1-3020: I.A + (J.1.8) + (A.3.31), C1-
3021: I.A + (J.1.11) + (A.3.31), C1-3022: I.A + (J.1.12) + (A.3.31), C1-3023: I.A + (K.1.3) +
(A.3.31), C1-3024: I.A + (K.1.29) + (A.3.31), C1-3025: I.A + (K.1.30) + (A.3.31), C1-3026: I.A +
(K.1.31) + (A.3.31), C1-3027: I.A + (K.1.32) + (A.3.31), C1-3028: I.A + (K.1.41) + (A.3.31), C1-
3029: I.A + (K.1.42) + (A.3.31), C1-3030: I.A + (K.1.44) + (A.3.31), C1-3031: I.A + (K.1.45) +
(A.3.31), C1-3032: I.A + (K.1.47) + (A.3.31), C1-3033: I.A + (K.1.49) + (A.3.31), C1-3034: I.A +
(K.1.53) + (A.3.31), C1-3035: I.A + (K.1.54) + (A.3.31), C1-3036: I.A + (A.1.4) + (A.3.32), C1-
3037: I.A + (A.1.12) + (A.3.32), C1-3038: I.A + (A.1.21) + (A.3.32), C1-3039: I.A + (A.1,21a) +
(A.3.32), C1-3040: I.A + (A.1.24) + (A.3.32), C1-3041: I.A + (A.1.26) + (A.3.32), C1-3042: I.A +
(A.1.27) + (A.3.32), C1-3043: I.A + (A.1.30) + (A.3.32), C1-3044: I.A + (A.1.31) + (A.3.32), C1-
3045: I.A + (A.1.32) + (A.3.32), C1-3046: I.A + (A.1.35) + (A.3.32), C1-3047: I.A + (A.2.1) +
(A.3.32), C1-3048: I.A + (A.2.3) + (A.3.32), C1-3049: I.A + (A.2.5) + (A.3.32), C1-3050: I.A +
(A.3.11) + (A.3.32), C1-3051: I.A + (A.3.12) + (A.3.32), C1-3052: I.A + (A.3.15) + (A.3.32), C1-
3053: I.A + (A.3.16) + (A.3.32), C1-3054: I.A + (A.3.18) + (A.3.32), C1-3055: I.A + (A.3.19) +
(A.3.32), C1-3056: I.A + (A.3.20) + (A.3.32), C1-3057: I.A + (A.3.21) + (A.3.32), C1-3058: I.A +
(A.3.22) + (A.3.32), C1-3059: I.A + (A.3.23) + (A.3.32), C1-3060: I.A + (A.3.25) + (A.3.32), C1-
3061: I.A + (A.3.26) + (A.3.32), C1-3062: I.A + (A.3.27) + (A.3.32), C1-3063: I.A + (A.3.33) +
(A.3.32), C1-3064: I.A + (A.3.34) + (A.3.32), C1-3065: I.A + (A.3.35) + (A.3.32), C1-3066: I.A +
(A.3.36) + (A.3.32), C1-3067: I.A + (A.3.37) + (A.3.32), C1-3068: I.A + (A.3.38) + (A.3.32), C1-
3069: I.A + (A.3.39) + (A.3.32), C1-3070: I.A + (A.4.5) + (A.3.32), C1-3071: I.A + (A.4.11) +
(A.3.32), C1-3072: I.A + (B.1.4) + (A.3.32), C1-3073: I.A + (B.1.5) + (A.3.32), C1-3074: I.A +
(B.1.8.) + (A.3.32), C1-3075: I.A + (B.1.10) + (A.3.32), C1-3076: I.A + (B.1.11) + (A.3.32), C1-
3077: I.A + (B.1.12) + (A.3.32), C1-3078: I.A + (B.1.13) + (A.3.32), C1-3079: I.A + (B.1.17) +
(A.3.32), C1-3080: I.A + (B.1.18) + (A.3.32), C1-3081: I.A + (B.1.21) + (A.3.32), C1-3082: I.A +
(B.1.22) + (A.3.32), C1-3083: I.A + (B.1.23) + (A.3.32), C1-3084: I.A + (B.1.25) + (A.3.32), C1-
3085: I.A + (B.1.26) + (A.3.32), C1-3086: I.A + (B.1.29) + (A.3.32), C1-3087: I.A + (B.1.33) +
(A.3.32), C1-3088: I.A + (B.1.34) + (A.3.32), C1-3089: I.A + (B.1.37) + (A.3.32), C1-3090: I.A +
(B.1.38) + (A.3.32), C1-3091: I.A + (B.1.43) + (A.3.32), C1-3092: I.A + (B.1.46) + (A.3.32), C1-
3093: I.A + (B.2.4) + (A.3.32), C1-3094: I.A + (B.2.5) + (A.3.32), C1-3095: I.A + (B.2.6) +
(A.3.32), C1-3096: I.A + (B.2.8) + (A.3.32), C1-3097: I.A + (C.1.1) + (A.3.32), C1-3098: I.A +
(C.1.2) + (A.3.32), C1-3099: I.A + (C.1.4) + (A.3.32), C1-3100: I.A + (C.1.5) + (A.3.32), C1-
3101: I.A + (C.2.6) + (A.3.32), C1-3102: I.A + (C.2.7) + (A.3.32), C1-3103: I.A + (C.2.8) +
(A.3.32), C1-3104: I.A + (D.1.1) + (A.3.32), C1-3105: I.A + (D.1.2) + (A.3.32), C1-3106: I.A +
(D.1.5) + (A.3.32), C1-3107: I.A + (D.2.4) + (A.3.32), C1-3108: I.A + (D.2.6) + (A.3.32), C1-
3109: I.A + (D.2.7) + (A.3.32), C1-3110: I.A + (E.1.1) + (A.3.32), C1-3111: I.A + (E.1.3) +
(A.3.32), C1-3112: I.A + (E.2.2) + (A.3.32), C1-3113: I.A + (E.2.3) + (A.3.32), C1-3114: I.A +
(F.1.2) + (A.3.32), C1-3115: I.A + (F.1.4) + (A.3.32), C1-3116: I.A + (F.1.5) + (A.3.32), C1-3117:
I.A + (G.3.1) + (A.3.32), C1-3118: I.A + (G.3.3) + (A.3.32), C1-3119: I.A + (G.3.6) + (A.3.32),
C1-3120: I.A + (G.5.1) + (A.3.32), C1-3121: I.A + (G.5.2) + (A.3.32), C1-3122: I.A + (G.5.3) +
(A.3.32), C1-3123: I.A + (G.5.4) + (A.3.32), C1-3124: I.A + (G.5.5) + (A.3.32), C1-3125: I.A +
(G.5.6) + (A.3.32), C1-3126: I.A + (G.5.7) + (A.3.32), C1-3127: I.A + (G.5.8) + (A.3.32), C1-
3128: I.A + (G.5.9) + (A.3.32), C1-3129: I.A + (G.5.10) + (A.3.32), C1-3130: I.A + (G.5.11) +
(A.3.32), C1-3131: I.A + (H.1.5) + (A.3.32), C1-3132: I.A + (H.1.7) + (A.3.32), C1-3133: I.A +
(H.2.2) + (A.3.32), C1-3134: I.A + (H.2.3) + (A.3.32), C1-3135: I.A + (H.2.5) + (A.3.32), C1-
3136: I.A + (H.2.7) + (A.3.32), C1-3137: I.A + (H.2.8) + (A.3.32), C1-3138: I.A + (H.3.2) +
(A.3.32), C1-3139: I.A + (H.3.4) + (A.3.32), C1-3140: I.A + (H.3.5) + (A.3.32), C1-3141: I.A +
(H.4.9) + (A.3.32), C1-3142: I.A + (H.4.10) + (A.3.32), C1-3143: I.A + (I.2.2) + (A.3.32), C1-
3144: I.A + (I.2.5) + (A.3.32), C1-3145: I.A + (J.1.2) + (A.3.32), C1-3146: I.A + (J.1.5) + (A.3.32),
C1-3147: I.A + (J.1.8) + (A.3.32), C1-3148: I.A + (J.1.11) + (A.3.32), C1-3149: I.A + (J.1.12) +
(A.3.32), C1-3150: I.A + (K.1.3) + (A.3.32), C1-3151: I.A + (K.1.29) + (A.3.32), C1-3152: I.A +
(K.1.30) + (A.3.32), C1-3153: I.A + (K.1.31) + (A.3.32), C1-3154: I.A + (K.1.32) + (A.3.32), C1-
3155: I.A + (K.1.41) + (A.3.32), C1-3156: I.A + (K.1.42) + (A.3.32), C1-3157: I.A + (K.1.44) +
(A.3.32), C1-3158: I.A + (K.1.45) + (A.3.32), C1-3159: I.A + (K.1.47) + (A.3.32), C1-3160: I.A +
(K.1.49) + (A.3.32), C1-3161: I.A + (K.1.53) + (A.3.32), C1-3162: I.A + (K.1.54) + (A.3.32), C1-
3163: I.A + (A.1.4) + (A.3.33), C1-3164: I.A + (A.1.12) + (A.3.33), C1-3165: I.A + (A.1.21) +
(A.3.33), C1-3166: I.A + (A.1,21a) + (A.3.33), C1-3167: I.A + (A.1.24) + (A.3.33), C1-3168: I.A +
(A.1.26) + (A.3.33), C1-3169: I.A + (A.1.27) + (A.3.33), C1-3170: I.A + (A.1.30) + (A.3.33), C1-
3171: I.A + (A.1.31) + (A.3.33), C1-3172: I.A + (A.1.32) + (A.3.33), C1-3173: I.A + (A.1.35) +
(A.3.33), C1-3174: I.A + (A.2.1) + (A.3.33), C1-3175: I.A + (A.2.3) + (A.3.33), C1-3176: I.A +
(A.2.5) + (A.3.33), C1-3177: I.A + (A.3.11) + (A.3.33), C1-3178: I.A + (A.3.12) + (A.3.33), C1-
3179: I.A + (A.3.15) + (A.3.33), C1-3180: I.A + (A.3.16) + (A.3.33), C1-3181: I.A + (A.3.18) +
(A.3.33), C1-3182: I.A + (A.3.19) + (A.3.33), C1-3183: I.A + (A.3.20) + (A.3.33), C1-3184: I.A +
(A.3.21) + (A.3.33), C1-3185: I.A + (A.3.22) + (A.3.33), C1-3186: I.A + (A.3.23) + (A.3.33), C1-
3187: I.A + (A.3.25) + (A.3.33), C1-3188: I.A + (A.3.26) + (A.3.33), C1-3189: I.A + (A.3.27) +
(A.3.33), C1-3190: I.A + (A.3.34) + (A.3.33), C1-3191: I.A + (A.3.35) + (A.3.33), C1-3192: I.A +
(A.3.36) + (A.3.33), C1-3193: I.A + (A.3.37) + (A.3.33), C1-3194: I.A + (A.3.38) + (A.3.33), C1-
3195: I.A + (A.3.39) + (A.3.33), C1-3196: I.A + (A.4.5) + (A.3.33), C1-3197: I.A + (A.4.11) +
(A.3.33), C1-3198: I.A + (B.1.4) + (A.3.33), C1-3199: I.A + (B.1.5) + (A.3.33), C1-3200: I.A +
(B.1.8.) + (A.3.33), C1-3201: I.A + (B.1.10) + (A.3.33), C1-3202: I.A + (B.1.11) + (A.3.33), C1-
3203: I.A + (B.1.12) + (A.3.33), C1-3204: I.A + (B.1.13) + (A.3.33), C1-3205: I.A + (B.1.17) +
(A.3.33), C1-3206: I.A + (B.1.18) + (A.3.33), C1-3207: I.A + (B.1.21) + (A.3.33), C1-3208: I.A +
(B.1.22) + (A.3.33), C1-3209: I.A + (B.1.23) + (A.3.33), C1-3210: I.A + (B.1.25) + (A.3.33), C1-
3211: I.A + (B.1.26) + (A.3.33), C1-3212: I.A + (B.1.29) + (A.3.33), C1-3213: I.A + (B.1.33) +
(A.3.33), C1-3214: I.A + (B.1.34) + (A.3.33), C1-3215: I.A + (B.1.37) + (A.3.33), C1-3216: I.A +
(B.1.38) + (A.3.33), C1-3217: I.A + (B.1.43) + (A.3.33), C1-3218: I.A + (B.1.46) + (A.3.33), C1-
3219: I.A + (B.2.4) + (A.3.33), C1-3220: I.A + (B.2.5) + (A.3.33), C1-3221: I.A + (B.2.6) +
(A.3.33), C1-3222: I.A + (B.2.8) + (A.3.33), C1-3223: I.A + (C.1.1) + (A.3.33), C1-3224: I.A +
(C.1.2) + (A.3.33), C1-3225: I.A + (C.1.4) + (A.3.33), C1-3226: I.A + (C.1.5) + (A.3.33), C1-
3227: I.A + (C.2.6) + (A.3.33), C1-3228: I.A + (C.2.7) + (A.3.33), C1-3229: I.A + (C.2.8) +
(A.3.33), C1-3230: I.A + (D.1.1) + (A.3.33), C1-3231: I.A + (D.1.2) + (A.3.33), C1-3232: I.A +
(D.1.5) + (A.3.33), C1-3233: I.A + (D.2.4) + (A.3.33), C1-3234: I.A + (D.2.6) + (A.3.33), C1-
3235: I.A + (D.2.7) + (A.3.33), C1-3236: I.A + (E.1.1) + (A.3.33), C1-3237: I.A + (E.1.3) +
(A.3.33), C1-3238: I.A + (E.2.2) + (A.3.33), C1-3239: I.A + (E.2.3) + (A.3.33), C1-3240: I.A +
(F.1.2) + (A.3.33), C1-3241: I.A + (F.1.4) + (A.3.33), C1-3242: I.A + (F.1.5) + (A.3.33), C1-3243:
I.A + (G.3.1) + (A.3.33), C1-3244: I.A + (G.3.3) + (A.3.33), C1-3245: I.A + (G.3.6) + (A.3.33),
C1-3246: I.A + (G.5.1) + (A.3.33), C1-3247: I.A + (G.5.2) + (A.3.33), C1-3248: I.A + (G.5.3) +
(A.3.33), C1-3249: I.A + (G.5.4) + (A.3.33), C1-3250: I.A + (G.5.5) + (A.3.33), C1-3251: I.A +
(G.5.6) + (A.3.33), C1-3252: I.A + (G.5.7) + (A.3.33), C1-3253: I.A + (G.5.8) + (A.3.33), C1-
3254: I.A + (G.5.9) + (A.3.33), C1-3255: I.A + (G.5.10) + (A.3.33), C1-3256: I.A + (G.5.11) +
(A.3.33), C1-3257: I.A + (H.1.5) + (A.3.33), C1-3258: I.A + (H.1.7) + (A.3.33), C1-3259: I.A +
(H.2.2) + (A.3.33), C1-3260: I.A + (H.2.3) + (A.3.33), C1-3261: I.A + (H.2.5) + (A.3.33), C1-
3262: I.A + (H.2.7) + (A.3.33), C1-3263: I.A + (H.2.8) + (A.3.33), C1-3264: I.A + (H.3.2) +
(A.3.33), C1-3265: I.A + (H.3.4) + (A.3.33), C1-3266: I.A + (H.3.5) + (A.3.33), C1-3267: I.A +
(H.4.9) + (A.3.33), C1-3268: I.A + (H.4.10) + (A.3.33), C1-3269: I.A + (I.2.2) + (A.3.33), C1-
3270: I.A + (I.2.5) + (A.3.33), C1-3271: I.A + (J. 1.2) + (A.3.33), C1-3272: I.A + (J. 1.5) + (A.3.33),
C1-3273: I.A + (J.1.8) + (A.3.33), C1-3274: I.A + (J.1.11) + (A.3.33), C1-3275: I.A + (J.1.12) +
(A.3.33), C1-3276: I.A + (K.1.3) + (A.3.33), C1-3277: I.A + (K.1.29) + (A.3.33), C1-3278: I.A +
(K.1.30) + (A.3.33), C1-3279: I.A + (K.1.31) + (A.3.33), C1-3280: I.A + (K.1.32) + (A.3.33), C1-
3281: I.A + (K.1.41) + (A.3.33), C1-3282: I.A + (K.1.42) + (A.3.33), C1-3283: I.A + (K.1.44) +
(A.3.33), C1-3284: I.A + (K.1.45) + (A.3.33), C1-3285: I.A + (K.1.47) + (A.3.33), C1-3286: I.A +
(K.1.49) + (A.3.33), C1-3287: I.A + (K.1.53) + (A.3.33), C1-3288: I.A + (K.1.54) + (A.3.33), C1-
3289: I.A + (A.1.4) + (A.3.34), C1-3290: I.A + (A.1.12) + (A.3.34), C1-3291: I.A + (A.1.21) +
(A.3.34), C1-3292: I.A + (A.1,21a) + (A.3.34), C1-3293: I.A + (A.1.24) + (A.3.34), C1-3294: I.A +
(A.1.26) + (A.3.34), C1-3295: I.A + (A.1.27) + (A.3.34), C1-3296: I.A + (A.1.30) + (A.3.34), C1-
3297: I.A + (A.1.31) + (A.3.34), C1-3298: I.A + (A.1.32) + (A.3.34), C1-3299: I.A + (A.1.35) +
(A.3.34), C1-3300: I.A + (A.2.1) + (A.3.34), C1-3301: I.A + (A.2.3) + (A.3.34), C1-3302: I.A +
(A.2.5) + (A.3.34), C1-3303: I.A + (A.3.11) + (A.3.34), C1-3304: I.A + (A.3.12) + (A.3.34), C1-
3305: I.A + (A.3.15) + (A.3.34), C1-3306: I.A + (A.3.16) + (A.3.34), C1-3307: I.A + (A.3.18) +
(A.3.34), C1-3308: I.A + (A.3.19) + (A.3.34), C1-3309: I.A + (A.3.20) + (A.3.34), C1-3310: I.A +
(A.3.21) + (A.3.34), C1-3311: I.A + (A.3.22) + (A.3.34), C1-3312: I.A + (A.3.23) + (A.3.34), C1-
3313: I.A + (A.3.25) + (A.3.34), C1-3314: I.A + (A.3.26) + (A.3.34), C1-3315: I.A + (A.3.27) +
(A.3.34), C1-3316: I.A + (A.3.35) + (A.3.34), C1-3317: I.A + (A.3.36) + (A.3.34), C1-3318: I.A +
(A.3.37) + (A.3.34), C1-3319: I.A + (A.3.38) + (A.3.34), C1-3320: I.A + (A.3.39) + (A.3.34), C1-
3321: I.A + (A.4.5) + (A.3.34), C1-3322: I.A + (A.4.11) + (A.3.34), C1-3323: I.A + (B.1.4) +
(A.3.34), C1-3324: I.A + (B.1.5) + (A.3.34), C1-3325: I.A + (B.1.8.) + (A.3.34), C1-3326: I.A +
(B.1.10) + (A.3.34), C1-3327: I.A + (B.1.11) + (A.3.34), C1-3328: I.A + (B.1.12) + (A.3.34), C1-
3329: I.A + (B.1.13) + (A.3.34), C1-3330: I.A + (B.1.17) + (A.3.34), C1-3331: I.A + (B.1.18) +
(A.3.34), C1-3332: I.A + (B.1.21) + (A.3.34), C1-3333: I.A + (B.1.22) + (A.3.34), C1-3334: I.A +
(B.1.23) + (A.3.34), C1-3335: I.A + (B.1.25) + (A.3.34), C1-3336: I.A + (B.1.26) + (A.3.34), C1-
3337: I.A + (B.1.29) + (A.3.34), C1-3338: I.A + (B.1.33) + (A.3.34), C1-3339: I.A + (B.1.34) +
(A.3.34), C1-3340: I.A + (B.1.37) + (A.3.34), C1-3341: I.A + (B.1.38) + (A.3.34), C1-3342: I.A +
(B.1.43) + (A.3.34), C1-3343: I.A + (B.1.46) + (A.3.34), C1-3344: I.A + (B.2.4) + (A.3.34), C1-
3345: I.A + (B.2.5) + (A.3.34), C1-3346: I.A + (B.2.6) + (A.3.34), C1-3347: I.A + (B.2.8) +
(A.3.34), C1-3348: I.A + (C.1.1) + (A.3.34), C1-3349: I.A + (C.1.2) + (A.3.34), C1-3350: I.A +
(C.1.4) + (A.3.34), C1-3351: I.A + (C.1.5) + (A.3.34), C1-3352: I.A + (C.2.6) + (A.3.34), C1-
3353: I.A + (C.2.7) + (A.3.34), C1-3354: I.A + (C.2.8) + (A.3.34), C1-3355: I.A + (D.1.1) +
(A.3.34), C1-3356: I.A + (D.1.2) + (A.3.34), C1-3357: I.A + (D.1.5) + (A.3.34), C1-3358: I.A +
(D.2.4) + (A.3.34), C1-3359: I.A + (D.2.6) + (A.3.34), C1-3360: I.A + (D.2.7) + (A.3.34), C1-
3361: I.A + (E.1.1) + (A.3.34), C1-3362: I.A + (E.1.3) + (A.3.34), C1-3363: I.A + (E.2.2) +
(A.3.34), C1-3364: I.A + (E.2.3) + (A.3.34), C1-3365: I.A + (F.1.2) + (A.3.34), C1-3366: I.A +
(F.1.4) + (A.3.34), C1-3367: I.A + (F.1.5) + (A.3.34), C1-3368: I.A + (G.3.1) + (A.3.34), C1-3369:
I.A + (G.3.3) + (A.3.34), C1-3370: I.A + (G.3.6) + (A.3.34), C1-3371: I.A + (G.5.1) + (A.3.34),
C1-3372: I.A + (G.5.2) + (A.3.34), C1-3373: I.A + (G.5.3) + (A.3.34), C1-3374: I.A + (G.5.4) +
(A.3.34), C1-3375: I.A + (G.5.5) + (A.3.34), C1-3376: I.A + (G.5.6) + (A.3.34), C1-3377: I.A +
(G.5.7) + (A.3.34), C1-3378: I.A + (G.5.8) + (A.3.34), C1-3379: I.A + (G.5.9) + (A.3.34), C1-
3380: I.A + (G.5.10) + (A.3.34), C1-3381: I.A + (G.5.11) + (A.3.34), C1-3382: I.A + (H.1.5) +
(A.3.34), C1-3383: I.A + (H.1.7) + (A.3.34), C1-3384: I.A + (H.2.2) + (A.3.34), C1-3385: I.A +
(H.2.3) + (A.3.34), C1-3386: I.A + (H.2.5) + (A.3.34), C1-3387: I.A + (H.2.7) + (A.3.34), C1-
3388: I.A + (H.2.8) + (A.3.34), C1-3389: I.A + (H.3.2) + (A.3.34), C1-3390: I.A + (H.3.4) +
(A.3.34), C1-3391: I.A + (H.3.5) + (A.3.34), C1-3392: I.A + (H.4.9) + (A.3.34), C1-3393: I.A +
(H.4.10) + (A.3.34), C1-3394: I.A + (I.2.2) + (A.3.34), C1-3395: I.A + (I.2.5) + (A.3.34), C1-3396:
I.A + (J.1.2) + (A.3.34), C1-3397: I.A + (J.1.5) + (A.3.34), C1-3398: I.A + (J.1.8) + (A.3.34), C1-
3399: I.A + (J.1.11) + (A.3.34), C1-3400: I.A + (J.1.12) + (A.3.34), C1-3401: I.A + (K.1.3) +
(A.3.34), C1-3402: I.A + (K.1.29) + (A.3.34), C1-3403: I.A + (K.1.30) + (A.3.34), C1-3404: I.A +
(K.1.31) + (A.3.34), C1-3405: I.A + (K.1.32) + (A.3.34), C1-3406: I.A + (K.1.41) + (A.3.34), C1-
3407: I.A + (K.1.42) + (A.3.34), C1-3408: I.A + (K.1.44) + (A.3.34), C1-3409: I.A + (K.1.45) +
(A.3.34), C1-3410: I.A + (K.1.47) + (A.3.34), C1-3411: I.A + (K.1.49) + (A.3.34), C1-3412: I.A +
(K.1.53) + (A.3.34), C1-3413: I.A + (K.1.54) + (A.3.34), C1-3414: I.A + (A.1.4) + (A.3.35), C1-
3415: I.A + (A.1.12) + (A.3.35), C1-3416: I.A + (A.1.21) + (A.3.35), C1-3417: I.A + (A.1,21a) +
(A.3.35), C1-3418: I.A + (A.1.24) + (A.3.35), C1-3419: I.A + (A.1.26) + (A.3.35), C1-3420: I.A +
(A.1.27) + (A.3.35), C1-3421: I.A + (A.1.30) + (A.3.35), C1-3422: I.A + (A.1.31) + (A.3.35), C1-
3423: I.A + (A.1.32) + (A.3.35), C1-3424: I.A + (A.1.35) + (A.3.35), C1-3425: I.A + (A.2.1) +
(A.3.35), C1-3426: I.A + (A.2.3) + (A.3.35), C1-3427: I.A + (A.2.5) + (A.3.35), C1-3428: I.A +
(A.3.11) + (A.3.35), C1-3429: I.A + (A.3.12) + (A.3.35), C1-3430: I.A + (A.3.15) + (A.3.35), C1-
3431: I.A + (A.3.16) + (A.3.35), C1-3432: I.A + (A.3.18) + (A.3.35), C1-3433: I.A + (A.3.19) +
(A.3.35), C1-3434: I.A + (A.3.20) + (A.3.35), C1-3435: I.A + (A.3.21) + (A.3.35), C1-3436: I.A +
(A.3.22) + (A.3.35), C1-3437: I.A + (A.3.23) + (A.3.35), C1-3438: I.A + (A.3.25) + (A.3.35), C1-
3439: I.A + (A.3.26) + (A.3.35), C1-3440: I.A + (A.3.27) + (A.3.35), C1-3441: I.A + (A.3.36) +
(A.3.35), C1-3442: I.A + (A.3.37) + (A.3.35), C1-3443: I.A + (A.3.38) + (A.3.35), C1-3444: I.A +
(A.3.39) + (A.3.35), C1-3445: I.A + (A.4.5) + (A.3.35), C1-3446: I.A + (A.4.11) + (A.3.35), C1-
3447: I.A + (B.1.4) + (A.3.35), C1-3448: I.A + (B.1.5) + (A.3.35), C1-3449: I.A + (B.1.8.) +
(A.3.35), C1-3450: I.A + (B.1.10) + (A.3.35), C1-3451: I.A + (B.1.11) + (A.3.35), C1-3452: I.A +
(B.1.12) + (A.3.35), C1-3453: I.A + (B.1.13) + (A.3.35), C1-3454: I.A + (B.1.17) + (A.3.35), C1-
3455: I.A + (B.1.18) + (A.3.35), C1-3456: I.A + (B.1.21) + (A.3.35), C1-3457: I.A + (B.1.22) +
(A.3.35), C1-3458: I.A + (B.1.23) + (A.3.35), C1-3459: I.A + (B.1.25) + (A.3.35), C1-3460: I.A +
(B.1.26) + (A.3.35), C1-3461: I.A + (B.1.29) + (A.3.35), C1-3462: I.A + (B.1.33) + (A.3.35), C1-
3463: I.A + (B.1.34) + (A.3.35), C1-3464: I.A + (B.1.37) + (A.3.35), C1-3465: I.A + (B.1.38) +
(A.3.35), C1-3466: I.A + (B.1.43) + (A.3.35), C1-3467: I.A + (B.1.46) + (A.3.35), C1-3468: I.A +
(B.2.4) + (A.3.35), C1-3469: I.A + (B.2.5) + (A.3.35), C1-3470: I.A + (B.2.6) + (A.3.35), C1-3471:
I.A + (B.2.8) + (A.3.35), C1-3472: I.A + (C.1.1) + (A.3.35), C1-3473: I.A + (C.1.2) + (A.3.35), C1-
3474: I.A + (C.1.4) + (A.3.35), C1-3475: I.A + (C.1.5) + (A.3.35), C1-3476: I.A + (C.2.6) +
(A.3.35), C1-3477: I.A + (C.2.7) + (A.3.35), C1-3478: I.A + (C.2.8) + (A.3.35), C1-3479: I.A +
(D.1.1) + (A.3.35), C1-3480: I.A + (D.1.2) + (A.3.35), C1-3481: I.A + (D.1.5) + (A.3.35), C1-
3482: I.A + (D.2.4) + (A.3.35), C1-3483: I.A + (D.2.6) + (A.3.35), C1-3484: I.A + (D.2.7) +
(A.3.35), C1-3485: I.A + (E.1.1) + (A.3.35), C1-3486: I.A + (E.1.3) + (A.3.35), C1-3487: I.A +
(E.2.2) + (A.3.35), C1-3488: I.A + (E.2.3) + (A.3.35), C1-3489: I.A + (F.1.2) + (A.3.35), C1-3490:
I.A + (F.1.4) + (A.3.35), C1-3491: I.A + (F.1.5) + (A.3.35), C1-3492: I.A + (G.3.1) + (A.3.35), C1-
3493: I.A + (G.3.3) + (A.3.35), C1-3494: I.A + (G.3.6) + (A.3.35), C1-3495: I.A + (G.5.1) +
(A.3.35), C1-3496: I.A + (G.5.2) + (A.3.35), C1-3497: I.A + (G.5.3) + (A.3.35), C1-3498: I.A +
(G.5.4) + (A.3.35), C1-3499: I.A + (G.5.5) + (A.3.35), C1-3500: I.A + (G.5.6) + (A.3.35), C1-
3501: I.A + (G.5.7) + (A.3.35), C1-3502: I.A + (G.5.8) + (A.3.35), C1-3503: I.A + (G.5.9) +
(A.3.35), C1-3504: I.A + (G.5.10) + (A.3.35), C1-3505: I.A + (G.5.11) + (A.3.35), C1-3506: I.A +
(H.1.5) + (A.3.35), C1-3507: I.A + (H.1.7) + (A.3.35), C1-3508: I.A + (H.2.2) + (A.3.35), C1-
3509: I.A + (H.2.3) + (A.3.35), C1-3510: I.A + (H.2.5) + (A.3.35), C1-3511: I.A + (H.2.7) +
(A.3.35), C1-3512: I.A + (H.2.8) + (A.3.35), C1-3513: I.A + (H.3.2) + (A.3.35), C1-3514: I.A +
(H.3.4) + (A.3.35), C1-3515: I.A + (H.3.5) + (A.3.35), C1-3516: I.A + (H.4.9) + (A.3.35), C1-
3517: I.A + (H.4.10) + (A.3.35), C1-3518: I.A + (I.2.2) + (A.3.35), C1-3519: I.A + (I.2.5) +
(A.3.35), C1-3520: I.A + (J.1.2) + (A.3.35), C1-3521: I.A + (J.1.5) + (A.3.35), C1-3522: I.A +
(J.1.8) + (A.3.35), C1-3523: I.A + (J.1.11) + (A.3.35), C1-3524: I.A + (J.1.12) + (A.3.35), C1-
3525: I.A + (K.1.3) + (A.3.35), C1-3526: I.A + (K.1.29) + (A.3.35), C1-3527: I.A + (K.1.30) +
(A.3.35), C1-3528: I.A + (K.1.31) + (A.3.35), C1-3529: I.A + (K.1.32) + (A.3.35), C1-3530: I.A +
(K.1.41) + (A.3.35), C1-3531: I.A + (K.1.42) + (A.3.35), C1-3532: I.A + (K.1.44) + (A.3.35), C1-
3533: I.A + (K.1.45) + (A.3.35), C1-3534: I.A + (K.1.47) + (A.3.35), C1-3535: I.A + (K.1.49) +
(A.3.35), C1-3536: I.A + (K.1.53) + (A.3.35), C1-3537: I.A + (K.1.54) + (A.3.35), C1-3538: I.A +
(A.1.4) + (A.3.36), C1-3539: I.A + (A.1.12) + (A.3.36), C1-3540: I.A + (A.1.21) + (A.3.36), C1-
3541: I.A + (A.1.21a) + (A.3.36), C1-3542: I.A + (A.1.24) + (A.3.36), C1-3543: I.A + (A.1.26) +
(A.3.36), C1-3544: I.A + (A.1.27) + (A.3.36), C1-3545: I.A + (A.1.30) + (A.3.36), C1-3546: I.A +
(A.1.31) + (A.3.36), C1-3547: I.A + (A.1.32) + (A.3.36), C1-3548: I.A + (A.1.35) + (A.3.36), C1-
3549: I.A + (A.2.1) + (A.3.36), C1-3550: I.A + (A.2.3) + (A.3.36), C1-3551: I.A + (A.2.5) +
(A.3.36), C1-3552: I.A + (A.3.11) + (A.3.36), C1-3553: I.A + (A.3.12) + (A.3.36), C1-3554: I.A +
(A.3.15) + (A.3.36), C1-3555: I.A + (A.3.16) + (A.3.36), C1-3556: I.A + (A.3.18) + (A.3.36), C1-
3557: I.A + (A.3.19) + (A.3.36), C1-3558: I.A + (A.3.20) + (A.3.36), C1-3559: I.A + (A.3.21) +
(A.3.36), C1-3560: I.A + (A.3.22) + (A.3.36), C1-3561: I.A + (A.3.23) + (A.3.36), C1-3562: I.A +
(A.3.25) + (A.3.36), C1-3563: I.A + (A.3.26) + (A.3.36), C1-3564: I.A + (A.3.27) + (A.3.36), C1-
3565: I.A + (A.3.37) + (A.3.36), C1-3566: I.A + (A.3.38) + (A.3.36), C1-3567: I.A + (A.3.39) +
(A.3.36), C1-3568: I.A + (A.4.5) + (A.3.36), C1-3569: I.A + (A.4.11) + (A.3.36), C1-3570: I.A +
(B.1.4) + (A.3.36), C1-3571: I.A + (B.1.5) + (A.3.36), C1-3572: I.A + (B.1.8.) + (A.3.36), C1-
3573: I.A + (B.1.10) + (A.3.36), C1-3574: I.A + (B.1.11) + (A.3.36), C1-3575: I.A + (B.1.12) +
(A.3.36), C1-3576: I.A + (B.1.13) + (A.3.36), C1-3577: I.A + (B.1.17) + (A.3.36), C1-3578: I.A +
(B.1.18) + (A.3.36), C1-3579: I.A + (B.1.21) + (A.3.36), C1-3580: I.A + (B.1.22) + (A.3.36), C1-
3581: I.A + (B.1.23) + (A.3.36), C1-3582: I.A + (B.1.25) + (A.3.36), C1-3583: I.A + (B.1.26) +
(A.3.36), C1-3584: I.A + (B.1.29) + (A.3.36), C1-3585: I.A + (B.1.33) + (A.3.36), C1-3586: I.A +
(B.1.34) + (A.3.36), C1-3587: I.A + (B.1.37) + (A.3.36), C1-3588: I.A + (B.1.38) + (A.3.36), C1-
3589: I.A + (B.1.43) + (A.3.36), C1-3590: I.A + (B.1.46) + (A.3.36), C1-3591: I.A + (B.2.4) +
(A.3.36), C1-3592: I.A + (B.2.5) + (A.3.36), C1-3593: I.A + (B.2.6) + (A.3.36), C1-3594: I.A +
(B.2.8) + (A.3.36), C1-3595: I.A + (C.1.1) + (A.3.36), C1-3596: I.A + (C.1.2) + (A.3.36), C1-3597:
I.A + (C.1.4) + (A.3.36), C1-3598: I.A + (C.1.5) + (A.3.36), C1-3599: I.A + (C.2.6) + (A.3.36), C1-
3600: I.A + (C.2.7) + (A.3.36), C1-3601: I.A + (C.2.8) + (A.3.36), C1-3602: I.A + (D.1.1) +
(A.3.36), C1-3603: I.A + (D.1.2) + (A.3.36), C1-3604: I.A + (D.1.5) + (A.3.36), C1-3605: I.A +
(D.2.4) + (A.3.36), C1-3606: I.A + (D.2.6) + (A.3.36), C1-3607: I.A + (D.2.7) + (A.3.36), C1-
3608: I.A + (E.1.1) + (A.3.36), C1-3609: I.A + (E.1.3) + (A.3.36), C1-3610: I.A + (E.2.2) +
(A.3.36), C1-3611: I.A + (E.2.3) + (A.3.36), C1-3612: I.A + (F.1.2) + (A.3.36), C1-3613: I.A +
(F.1.4) + (A.3.36), C1-3614: I.A + (F.1.5) + (A.3.36), C1-3615: I.A + (G.3.1) + (A.3.36), C1-3616:
I.A + (G.3.3) + (A.3.36), C1-3617: I.A + (G.3.6) + (A.3.36), C1-3618: I.A + (G.5.1) + (A.3.36),
C1-3619: I.A + (G.5.2) + (A.3.36), C1-3620: I.A + (G.5.3) + (A.3.36), C1-3621: I.A + (G.5.4) +
(A.3.36), C1-3622: I.A + (G.5.5) + (A.3.36), C1-3623: I.A + (G.5.6) + (A.3.36), C1-3624: I.A +
(G.5.7) + (A.3.36), C1-3625: I.A + (G.5.8) + (A.3.36), C1-3626: I.A + (G.5.9) + (A.3.36), C1-
3627: I.A + (G.5.10) + (A.3.36), C1-3628: I.A + (G.5.11) + (A.3.36), C1-3629: I.A + (H.1.5) +
(A.3.36), C1-3630: I.A + (H.1.7) + (A.3.36), C1-3631: I.A + (H.2.2) + (A.3.36), C1-3632: I.A +
(H.2.3) + (A.3.36), C1-3633: I.A + (H.2.5) + (A.3.36), C1-3634: I.A + (H.2.7) + (A.3.36), C1-
3635: I.A + (H.2.8) + (A.3.36), C1-3636: I.A + (H.3.2) + (A.3.36), C1-3637: I.A + (H.3.4) +
(A.3.36), C1-3638: I.A + (H.3.5) + (A.3.36), C1-3639: I.A + (H.4.9) + (A.3.36), C1-3640: I.A +
(H.4.10) + (A.3.36), C1-3641: I.A + (I.2.2) + (A.3.36), C1-3642: I.A + (I.2.5) + (A.3.36), C1-3643:
I.A + (J.1.2) + (A.3.36), C1-3644: I.A + (J.1.5) + (A.3.36), C1-3645: I.A + (J.1.8) + (A.3.36), C1-
3646: I.A + (J.1.11) + (A.3.36), C1-3647: I.A + (J.1.12) + (A.3.36), C1-3648: I.A + (K.1.3) +
(A.3.36), C1-3649: I.A + (K.1.29) + (A.3.36), C1-3650: I.A + (K.1.30) + (A.3.36), C1-3651: I.A +
(K.1.31) + (A.3.36), C1-3652: I.A + (K.1.32) + (A.3.36), C1-3653: I.A + (K.1.41) + (A.3.36), C1-
3654: I.A + (K.1.42) + (A.3.36), C1-3655: I.A + (K.1.44) + (A.3.36), C1-3656: I.A + (K.1.45) +
(A.3.36), C1-3657: I.A + (K.1.47) + (A.3.36), C1-3658: I.A + (K.1.49) + (A.3.36), C1-3659: I.A +
(K.1.53) + (A.3.36), C1-3660: I.A + (K.1.54) + (A.3.36), C1-3661: I.A + (A.1.4) + (A.3.37), C1-
3662: I.A + (A.1.12) + (A.3.37), C1-3663: I.A + (A.1.21) + (A.3.37), C1-3664: I.A + (A.1,21a) +
(A.3.37), C1-3665: I.A + (A.1.24) + (A.3.37), C1-3666: I.A + (A.1.26) + (A.3.37), C1-3667: I.A +
(A.1.27) + (A.3.37), C1-3668: I.A + (A.1.30) + (A.3.37), C1-3669: I.A + (A.1.31) + (A.3.37), C1-
3670: I.A + (A.1.32) + (A.3.37), C1-3671: I.A + (A.1.35) + (A.3.37), C1-3672: I.A + (A.2.1) +
(A.3.37), C1-3673: I.A + (A.2.3) + (A.3.37), C1-3674: I.A + (A.2.5) + (A.3.37), C1-3675: I.A +
(A.3.11) + (A.3.37), C1-3676: I.A + (A.3.12) + (A.3.37), C1-3677: I.A + (A.3.15) + (A.3.37), C1-
3678: I.A + (A.3.16) + (A.3.37), C1-3679: I.A + (A.3.18) + (A.3.37), C1-3680: I.A + (A.3.19) +
(A.3.37), C1-3681: I.A + (A.3.20) + (A.3.37), C1-3682: I.A + (A.3.21) + (A.3.37), C1-3683: I.A +
(A.3.22) + (A.3.37), C1-3684: I.A + (A.3.23) + (A.3.37), C1-3685: I.A + (A.3.25) + (A.3.37), C1-
3686: I.A + (A.3.26) + (A.3.37), C1-3687: I.A + (A.3.27) + (A.3.37), C1-3688: I.A + (A.3.38) +
(A.3.37), C1-3689: I.A + (A.3.39) + (A.3.37), C1-3690: I.A + (A.4.5) + (A.3.37), C1-3691: I.A +
(A.4.11) + (A.3.37), C1-3692: I.A + (B.1.4) + (A.3.37), C1-3693: I.A + (B.1.5) + (A.3.37), C1-
3694: I.A + (B.1.8.) + (A.3.37), C1-3695: I.A + (B.1.10) + (A.3.37), C1-3696: I.A + (B.1.11) +
(A.3.37), C1-3697: I.A + (B.1.12) + (A.3.37), C1-3698: I.A + (B.1.13) + (A.3.37), C1-3699: I.A +
(B.1.17) + (A.3.37), C1-3700: I.A + (B.1.18) + (A.3.37), C1-3701: I.A + (B.1.21) + (A.3.37), C1-
3702: I.A + (B.1.22) + (A.3.37), C1-3703: I.A + (B.1.23) + (A.3.37), C1-3704: I.A + (B.1.25) +
(A.3.37), C1-3705: I.A + (B.1.26) + (A.3.37), C1-3706: I.A + (B.1.29) + (A.3.37), C1-3707: I.A +
(B.1.33) + (A.3.37), C1-3708: I.A + (B.1.34) + (A.3.37), C1-3709: I.A + (B.1.37) + (A.3.37), C1-
3710: I.A + (B.1.38) + (A.3.37), C1-3711: I.A + (B.1.43) + (A.3.37), C1-3712: I.A + (B.1.46) +
(A.3.37), C1-3713: I.A + (B.2.4) + (A.3.37), C1-3714: I.A + (B.2.5) + (A.3.37), C1-3715: I.A +
(B.2.6) + (A.3.37), C1-3716: I.A + (B.2.8) + (A.3.37), C1-3717: I.A + (C.1.1) + (A.3.37), C1-3718:
I.A + (C.1.2) + (A.3.37), C1-3719: I.A + (C.1.4) + (A.3.37), C1-3720: I.A + (C.1.5) + (A.3.37), C1-
3721: I.A + (C.2.6) + (A.3.37), C1-3722: I.A + (C.2.7) + (A.3.37), C1-3723: I.A + (C.2.8) +
(A.3.37), C1-3724: I.A + (D.1.1) + (A.3.37), C1-3725: I.A + (D.1.2) + (A.3.37), C1-3726: I.A +
(D.1.5) + (A.3.37), C1-3727: I.A + (D.2.4) + (A.3.37), C1-3728: I.A + (D.2.6) + (A.3.37), C1-
3729: I.A + (D.2.7) + (A.3.37), C1-3730: I.A + (E.1.1) + (A.3.37), C1-3731: I.A + (E.1.3) +
(A.3.37), C1-3732: I.A + (E.2.2) + (A.3.37), C1-3733: I.A + (E.2.3) + (A.3.37), C1-3734: I.A +
(F.1.2) + (A.3.37), C1-3735: I.A + (F.1.4) + (A.3.37), C1-3736: I.A + (F.1.5) + (A.3.37), C1-3737:
I.A + (G.3.1) + (A.3.37), C1-3738: I.A + (G.3.3) + (A.3.37), C1-3739: I.A + (G.3.6) + (A.3.37),
C1-3740: I.A + (G.5.1) + (A.3.37), C1-3741: I.A + (G.5.2) + (A.3.37), C1-3742: I.A + (G.5.3) +
(A.3.37), C1-3743: I.A + (G.5.4) + (A.3.37), C1-3744: I.A + (G.5.5) + (A.3.37), C1-3745: I.A +
(G.5.6) + (A.3.37), C1-3746: I.A + (G.5.7) + (A.3.37), C1-3747: I.A + (G.5.8) + (A.3.37), C1-
3748: I.A + (G.5.9) + (A.3.37), C1-3749: I.A + (G.5.10) + (A.3.37), C1-3750: I.A + (G.5.11) +
(A.3.37), C1-3751: I.A + (H.1.5) + (A.3.37), C1-3752: I.A + (H.1.7) + (A.3.37), C1-3753: I.A +
(H.2.2) + (A.3.37), C1-3754: I.A + (H.2.3) + (A.3.37), C1-3755: I.A + (H.2.5) + (A.3.37), C1-
3756: I.A + (H.2.7) + (A.3.37), C1-3757: I.A + (H.2.8) + (A.3.37), C1-3758: I.A + (H.3.2) +
(A.3.37), C1-3759: I.A + (H.3.4) + (A.3.37), C1-3760: I.A + (H.3.5) + (A.3.37), C1-3761: I.A +
(H.4.9) + (A.3.37), C1-3762: I.A + (H.4.10) + (A.3.37), C1-3763: I.A + (I.2.2) + (A.3.37), C1-
3764: I.A + (I.2.5) + (A.3.37), C1-3765: I.A + (J.1.2) + (A.3.37), C1-3766: I.A + (J.1.5) + (A.3.37),
C1-3767: I.A + (J.1.8) + (A.3.37), C1-3768: I.A + (J.1.11) + (A.3.37), C1-3769: I.A + (J.1.12) +
(A.3.37), C1-3770: I.A + (K.1.3) + (A.3.37), C1-3771: I.A + (K.1.29) + (A.3.37), C1-3772: I.A +
(K.1.30) + (A.3.37), C1-3773: I.A + (K.1.31) + (A.3.37), C1-3774: I.A + (K.1.32) + (A.3.37), C1-
3775: I.A + (K.1.41) + (A.3.37), C1-3776: I.A + (K.1.42) + (A.3.37), C1-3777: I.A + (K.1.44) +
(A.3.37), C1-3778: I.A + (K.1.45) + (A.3.37), C1-3779: I.A + (K.1.47) + (A.3.37), C1-3780: I.A +
(K.1.49) + (A.3.37), C1-3781: I.A + (K.1.53) + (A.3.37), C1-3782: I.A + (K.1.54) + (A.3.37), C1-
3783: I.A + (A.1.4) + (A.3.38), C1-3784: I.A + (A.1.12) + (A.3.38), C1-3785: I.A + (A.1.21) +
(A.3.38), C1-3786: I.A + (A.1,21a) + (A.3.38), C1-3787: I.A + (A.1.24) + (A.3.38), C1-3788: I.A +
(A.1.26) + (A.3.38), C1-3789: I.A + (A.1.27) + (A.3.38), C1-3790: I.A + (A.1.30) + (A.3.38), C1-
3791: I.A + (A.1.31) + (A.3.38), C1-3792: I.A + (A.1.32) + (A.3.38), C1-3793: I.A + (A.1.35) +
(A.3.38), C1-3794: I.A + (A.2.1) + (A.3.38), C1-3795: I.A + (A.2.3) + (A.3.38), C1-3796: I.A +
(A.2.5) + (A.3.38), C1-3797: I.A + (A.3.11) + (A.3.38), C1-3798: I.A + (A.3.12) + (A.3.38), C1-
3799: I.A + (A.3.15) + (A.3.38), C1-3800: I.A + (A.3.16) + (A.3.38), C1-3801: I.A + (A.3.18) +
(A.3.38), C1-3802: I.A + (A.3.19) + (A.3.38), C1-3803: I.A + (A.3.20) + (A.3.38), C1-3804: I.A +
(A.3.21) + (A.3.38), C1-3805: I.A + (A.3.22) + (A.3.38), C1-3806: I.A + (A.3.23) + (A.3.38), C1-
3807: I.A + (A.3.25) + (A.3.38), C1-3808: I.A + (A.3.26) + (A.3.38), C1-3809: I.A + (A.3.27) +
(A.3.38), C1-3810: I.A + (A.3.39) + (A.3.38), C1-3811: I.A + (A.4.5) + (A.3.38), C1-3812: I.A +
(A.4.11) + (A.3.38), C1-3813: I.A + (B.1.4) + (A.3.38), C1-3814: I.A + (B.1.5) + (A.3.38), C1-
3815: I.A + (B.1.8.) + (A.3.38), C1-3816: I.A + (B.1.10) + (A.3.38), C1-3817: I.A + (B.1.11) +
(A.3.38), C1-3818: I.A + (B.1.12) + (A.3.38), C1-3819: I.A + (B.1.13) + (A.3.38), C1-3820: I.A +
(B.1.17) + (A.3.38), C1-3821: I.A + (B.1.18) + (A.3.38), C1-3822: I.A + (B.1.21) + (A.3.38), C1-
3823: I.A + (B.1.22) + (A.3.38), C1-3824: I.A + (B.1.23) + (A.3.38), C1-3825: I.A + (B.1.25) +
(A.3.38), C1-3826: I.A + (B.1.26) + (A.3.38), C1-3827: I.A + (B.1.29) + (A.3.38), C1-3828: I.A +
(B.1.33) + (A.3.38), C1-3829: I.A + (B.1.34) + (A.3.38), C1-3830: I.A + (B.1.37) + (A.3.38), C1-
3831: I.A + (B.1.38) + (A.3.38), C1-3832: I.A + (B.1.43) + (A.3.38), C1-3833: I.A + (B.1.46) +
(A.3.38), C1-3834: I.A + (B.2.4) + (A.3.38), C1-3835: I.A + (B.2.5) + (A.3.38), C1-3836: I.A +
(B.2.6) + (A.3.38), C1-3837: I.A + (B.2.8) + (A.3.38), C1-3838: I.A + (C.1.1) + (A.3.38), C1-3839:
I.A + (C.1.2) + (A.3.38), C1-3840: I.A + (C.1.4) + (A.3.38), C1-3841: I.A + (C.1.5) + (A.3.38), C1-
3842: I.A + (C.2.6) + (A.3.38), C1-3843: I.A + (C.2.7) + (A.3.38), C1-3844: I.A + (C.2.8) +
(A.3.38), C1-3845: I.A + (D.1.1) + (A.3.38), C1-3846: I.A + (D.1.2) + (A.3.38), C1-3847: I.A +
(D.1.5) + (A.3.38), C1-3848: I.A + (D.2.4) + (A.3.38), C1-3849: I.A + (D.2.6) + (A.3.38), C1-
3850: I.A + (D.2.7) + (A.3.38), C1-3851: I.A + (E.1.1) + (A.3.38), C1-3852: I.A + (E.1.3) +
(A.3.38), C1-3853: I.A + (E.2.2) + (A.3.38), C1-3854: I.A + (E.2.3) + (A.3.38), C1-3855: I.A +
(F.1.2) + (A.3.38), C1-3856: I.A + (F.1.4) + (A.3.38), C1-3857: I.A + (F.1.5) + (A.3.38), C1-3858:
I.A + (G.3.1) + (A.3.38), C1-3859: I.A + (G.3.3) + (A.3.38), C1-3860: I.A + (G.3.6) + (A.3.38),
C1-3861: I.A + (G.5.1) + (A.3.38), C1-3862: I.A + (G.5.2) + (A.3.38), C1-3863: I.A + (G.5.3) +
(A.3.38), C1-3864: I.A + (G.5.4) + (A.3.38), C1-3865: I.A + (G.5.5) + (A.3.38), C1-3866: I.A +
(G.5.6) + (A.3.38), C1-3867: I.A + (G.5.7) + (A.3.38), C1-3868: I.A + (G.5.8) + (A.3.38), C1-
3869: I.A + (G.5.9) + (A.3.38), C1-3870: I.A + (G.5.10) + (A.3.38), C1-3871: I.A + (G.5.11) +
(A.3.38), C1-3872: I.A + (H.1.5) + (A.3.38), C1-3873: I.A + (H.1.7) + (A.3.38), C1-3874: I.A +
(H.2.2) + (A.3.38), C1-3875: I.A + (H.2.3) + (A.3.38), C1-3876: I.A + (H.2.5) + (A.3.38), C1-
3877: I.A + (H.2.7) + (A.3.38), C1-3878: I.A + (H.2.8) + (A.3.38), C1-3879: I.A + (H.3.2) +
(A.3.38), C1-3880: I.A + (H.3.4) + (A.3.38), C1-3881: I.A + (H.3.5) + (A.3.38), C1-3882: I.A +
(H.4.9) + (A.3.38), C1-3883: I.A + (H.4.10) + (A.3.38), C1-3884: I.A + (I.2.2) + (A.3.38), C1-
3885: I.A + (I.2.5) + (A.3.38), C1-3886: I.A + (J.1.2) + (A.3.38), C1-3887: I.A + (J.1.5) + (A.3.38),
C1-3888: I.A + (J.1.8) + (A.3.38), C1-3889: I.A + (J.1.11) + (A.3.38), C1-3890: I.A + (J.1.12) +
(A.3.38), C1-3891: I.A + (K.1.3) + (A.3.38), C1-3892: I.A + (K.1.29) + (A.3.38), C1-3893: I.A +
(K.1.30) + (A.3.38), C1-3894: I.A + (K.1.31) + (A.3.38), C1-3895: I.A + (K.1.32) + (A.3.38), C1-
3896: I.A + (K.1.41) + (A.3.38), C1-3897: I.A + (K.1.42) + (A.3.38), C1-3898: I.A + (K.1.44) +
(A.3.38), C1-3899: I.A + (K.1.45) + (A.3.38), C1-3900: I.A + (K.1.47) + (A.3.38), C1-3901: I.A +
(K.1.49) + (A.3.38), C1-3902: I.A + (K.1.53) + (A.3.38), C1-3903: I.A + (K.1.54) + (A.3.38), C1-
3904: I.A + (A.1.4) + (A.3.39), C1-3905: I.A + (A.1.12) + (A.3.39), C1-3906: I.A + (A.1.21) +
(A.3.39), C1-3907: I.A + (A.1,21a) + (A.3.39), C1-3908: I.A + (A.1.24) + (A.3.39), C1-3909: I.A +
(A.1.26) + (A.3.39), C1-3910: I.A + (A.1.27) + (A.3.39), C1-3911: I.A + (A.1.30) + (A.3.39), C1-
3912: I.A + (A.1.31) + (A.3.39), C1-3913: I.A + (A.1.32) + (A.3.39), C1-3914: I.A + (A.1.35) +
(A.3.39), C1-3915: I.A + (A.2.1) + (A.3.39), C1-3916: I.A + (A.2.3) + (A.3.39), C1-3917: I.A +
(A.2.5) + (A.3.39), C1-3918: I.A + (A.3.11) + (A.3.39), C1-3919: I.A + (A.3.12) + (A.3.39), C1-
3920: I.A + (A.3.15) + (A.3.39), C1-3921: I.A + (A.3.16) + (A.3.39), C1-3922: I.A + (A.3.18) +
(A.3.39), C1-3923: I.A + (A.3.19) + (A.3.39), C1-3924: I.A + (A.3.20) + (A.3.39), C1-3925: I.A +
(A.3.21) + (A.3.39), C1-3926: I.A + (A.3.22) + (A.3.39), C1-3927: I.A + (A.3.23) + (A.3.39), C1-
3928: I.A + (A.3.25) + (A.3.39), C1-3929: I.A + (A.3.26) + (A.3.39), C1-3930: I.A + (A.3.27) +
(A.3.39), C1-3931: I.A + (A.4.5) + (A.3.39), C1-3932: I.A + (A.4.11) + (A.3.39), C1-3933: I.A +
(B.1.4) + (A.3.39), C1-3934: I.A + (B.1.5) + (A.3.39), C1-3935: I.A + (B.1.8.) + (A.3.39), C1-
3936: I.A + (B.1.10) + (A.3.39), C1-3937: I.A + (B.1.11) + (A.3.39), C1-3938: I.A + (B.1.12) +
(A.3.39), C1-3939: I.A + (B.1.13) + (A.3.39), C1-3940: I.A + (B.1.17) + (A.3.39), C1-3941: I.A +
(B.1.18) + (A.3.39), C1-3942: I.A + (B.1.21) + (A.3.39), C1-3943: I.A + (B.1.22) + (A.3.39), C1-
3944: I.A + (B.1.23) + (A.3.39), C1-3945: I.A + (B.1.25) + (A.3.39), C1-3946: I.A + (B.1.26) +
(A.3.39), C1-3947: I.A + (B.1.29) + (A.3.39), C1-3948: I.A + (B.1.33) + (A.3.39), C1-3949: I.A +
(B.1.34) + (A.3.39), C1-3950: I.A + (B.1.37) + (A.3.39), C1-3951: I.A + (B.1.38) + (A.3.39), C1-
3952: I.A + (B.1.43) + (A.3.39), C1-3953: I.A + (B.1.46) + (A.3.39), C1-3954: I.A + (B.2.4) +
(A.3.39), C1-3955: I.A + (B.2.5) + (A.3.39), C1-3956: I.A + (B.2.6) + (A.3.39), C1-3957: I.A +
(B.2.8) + (A.3.39), C1-3958: I.A + (C.1.1) + (A.3.39), C1-3959: I.A + (C.1.2) + (A.3.39), C1-3960:
I.A + (C.1.4) + (A.3.39), C1-3961: I.A + (C.1.5) + (A.3.39), C1-3962: I.A + (C.2.6) + (A.3.39), C1-
3963: I.A + (C.2.7) + (A.3.39), C1-3964: I.A + (C.2.8) + (A.3.39), C1-3965: I.A + (D.1.1) +
(A.3.39), C1-3966: I.A + (D.1.2) + (A.3.39), C1-3967: I.A + (D.1.5) + (A.3.39), C1-3968: I.A +
(D.2.4) + (A.3.39), C1-3969: I.A + (D.2.6) + (A.3.39), C1-3970: I.A + (D.2.7) + (A.3.39), C1-
3971: I.A + (E.1.1) + (A.3.39), C1-3972: I.A + (E.1.3) + (A.3.39), C1-3973: I.A + (E.2.2) +
(A.3.39), C1-3974: I.A + (E.2.3) + (A.3.39), C1-3975: I.A + (F.1.2) + (A.3.39), C1-3976: I.A +
(F.1.4) + (A.3.39), C1-3977: I.A + (F.1.5) + (A.3.39), C1-3978: I.A + (G.3.1) + (A.3.39), C1-3979:
I.A + (G.3.3) + (A.3.39), C1-3980: I.A + (G.3.6) + (A.3.39), C1-3981: I.A + (G.5.1) + (A.3.39),
C1-3982: I.A + (G.5.2) + (A.3.39), C1-3983: I.A + (G.5.3) + (A.3.39), C1-3984: I.A + (G.5.4) +
(A.3.39), C1-3985: I.A + (G.5.5) + (A.3.39), C1-3986: I.A + (G.5.6) + (A.3.39), C1-3987: I.A +
(G.5.7) + (A.3.39), C1-3988: I.A + (G.5.8) + (A.3.39), C1-3989: I.A + (G.5.9) + (A.3.39), C1-
3990: I.A + (G.5.10) + (A.3.39), C1-3991: I.A + (G.5.11) + (A.3.39), C1-3992: I.A + (H.1.5) +
(A.3.39), C1-3993: I.A + (H.1.7) + (A.3.39), C1-3994: I.A + (H.2.2) + (A.3.39), C1-3995: I.A +
(H.2.3) + (A.3.39), C1-3996: I.A + (H.2.5) + (A.3.39), C1-3997: I.A + (H.2.7) + (A.3.39), C1-
3998: I.A + (H.2.8) + (A.3.39), C1-3999: I.A + (H.3.2) + (A.3.39), C1-4000: I.A + (H.3.4) +
(A.3.39), C1-4001: I.A + (H.3.5) + (A.3.39), C1-4002: I.A + (H.4.9) + (A.3.39), C1-4003: I.A +
(H.4.10) + (A.3.39), C1-4004: I.A + (I.2.2) + (A.3.39), C1-4005: I.A + (I.2.5) + (A.3.39), C1-4006:
I.A + (J.1.2) + (A.3.39), C1-4007: I.A + (J.1.5) + (A.3.39), C1-4008: I.A + (J.1.8) + (A.3.39), C1-
4009: I.A + (J.1.11) + (A.3.39), C1-4010: I.A + (J.1.12) + (A.3.39), C1-4011: I.A + (K.1.3) +
(A.3.39), C1-4012: I.A + (K.1.29) + (A.3.39), C1-4013: I.A + (K.1.30) + (A.3.39), C1-4014: I.A +
(K.1.31) + (A.3.39), C1-4015: I.A + (K.1.32) + (A.3.39), C1-4016: I.A + (K.1.41) + (A.3.39), C1-
4017: I.A + (K.1.42) + (A.3.39), C1-4018: I.A + (K.1.44) + (A.3.39), C1-4019: I.A + (K.1.45) +
(A.3.39), C1-4020: I.A + (K.1.47) + (A.3.39), C1-4021: I.A + (K.1.49) + (A.3.39), C1-4022: I.A +
(K.1.53) + (A.3.39), C1-4023: I.A + (K.1.54) + (A.3.39), C1-4024: I.A + (A.1.4) + (B.1.4), C1-
4025: I.A + (A.1.12) + (B.1.4), C1-4026: I.A + (A.1.21) + (B.1.4), C1-4027: I.A + (A.1,21a) +
(B.1.4), C1-4028: I.A + (A.1.24) + (B.1.4), C1-4029: I.A + (A.1.26) + (B.1.4), C1-4030: I.A +
(A.1.27) + (B.1.4), C1-4031: I.A + (A.1.30) + (B.1.4), C1-4032: I.A + (A.1.31) + (B.1.4), C1-4033:
I.A + (A.1.32) + (B.1.4), C1-4034: I.A + (A.1.35) + (B.1.4), C1-4035: I.A + (A.2.1) + (B.1.4), C1-
4036: I.A + (A.2.3) + (B.1.4), C1-4037: I.A + (A.2.5) + (B.1.4), C1-4038: I.A + (A.3.11) + (B.1.4),
C1-4039: I.A + (A.3.12) + (B.1.4), C1-4040: I.A + (A.3.15) + (B.1.4), C1-4041: I.A + (A.3.16) +
(B.1.4), C1-4042: I.A + (A.3.18) + (B.1.4), C1-4043: I.A + (A.3.19) + (B.1.4), C1-4044: I.A +
(A.3.20) + (B.1.4), C1-4045: I.A + (A.3.21) + (B.1.4), C1-4046: I.A + (A.3.22) + (B.1.4), C1-4047:
I.A + (A.3.23) + (B.1.4), C1-4048: I.A + (A.3.25) + (B.1.4), C1-4049: I.A + (A.3.26) + (B.1.4), C1-
4050: I.A + (A.3.27) + (B.1.4), C1-4051: I.A + (A.4.5) + (B.1.4), C1-4052: I.A + (A.4.11) +
(B.1.4), C1-4053: I.A + (B.1.5) + (B.1.4), C1-4054: I.A + (B.1.8.) + (B.1.4), C1-4055: I.A +
(B.1.10) + (B.1.4), C1-4056: I.A + (B.1.11) + (B.1.4), C1-4057: I.A + (B.1.12) + (B.1.4), C1-4058:
I.A + (B.1.13) + (B.1.4), C1-4059: I.A + (B.1.17) + (B.1.4), C1-4060: I.A + (B.1.18) + (B.1.4), C1-
4061: I.A + (B.1.21) + (B.1.4), C1-4062: I.A + (B.1.22) + (B.1.4), C1-4063: I.A + (B.1.23) +
(B.1.4), C1-4064: I.A + (B.1.25) + (B.1.4), C1-4065: I.A + (B.1.26) + (B.1.4), C1-4066: I.A +
(B.1.29) + (B.1.4), C1-4067: I.A + (B.1.33) + (B.1.4), C1-4068: I.A + (B.1.34) + (B.1.4), C1-4069:
I.A + (B.1.37) + (B.1.4), C1-4070: I.A + (B.1.38) + (B.1.4), C1-4071: I.A + (B.1.43) + (B.1.4), C1-
4072: I.A + (B.1.46) + (B.1.4), C1-4073: I.A + (B.2.4) + (B.1.4), C1-4074: I.A + (B.2.5) + (B.1.4),
C1-4075: I.A + (B.2.6) + (B.1.4), C1-4076: I.A + (B.2.8) + (B.1.4), C1-4077: I.A + (C.1.1) +
(B.1.4), C1-4078: I.A + (C.1.2) + (B.1.4), C1-4079: I.A + (C.1.4) + (B.1.4), C1-4080: I.A + (C.1.5) +
(B.1.4), C1-4081: I.A + (C.2.6) + (B.1.4), C1-4082: I.A + (C.2.7) + (B.1.4), C1-4083: I.A +
(C.2.8) + (B.1.4), C1-4084: I.A + (D.1.1) + (B.1.4), C1-4085: I.A + (D.1.2) + (B.1.4), C1-4086: I.A +
(D.1.5) + (B.1.4), C1-4087: I.A + (D.2.4) + (B.1.4), C1-4088: I.A + (D.2.6) + (B.1.4), C1-4089:
I.A + (D.2.7) + (B.1.4), C1-4090: I.A + (E.1.1) + (B.1.4), C1-4091: I.A + (E.1.3) + (B.1.4), C1-
4092: I.A + (E.2.2) + (B.1.4), C1-4093: I.A + (E.2.3) + (B.1.4), C1-4094: I.A + (F.1.2) + (B.1.4),
C1-4095: I.A + (F.1.4) + (B.1.4), C1-4096: I.A + (F.1.5) + (B.1.4), C1-4097: I.A + (G.3.1) +
(B.1.4), C1-4098: I.A + (G.3.3) + (B.1.4), C1-4099: I.A + (G.3.6) + (B.1.4), C1-4100: I.A +
(G.5.1) + (B.1.4), C1-4101: I.A + (G.5.2) + (B.1.4), C1-4102: I.A + (G.5.3) + (B.1.4), C1-4103:
I.A + (G.5.4) + (B.1.4), C1-4104: I.A + (G.5.5) + (B.1.4), C1-4105: I.A + (G.5.6) + (B.1.4), C1-
4106: I.A + (G.5.7) + (B.1.4), C1-4107: I.A + (G.5.8) + (B.1.4), C1-4108: I.A + (G.5.9) + (B.1.4),
C1-4109: I.A + (G.5.10) + (B.1.4), C1-4110: I.A + (G.5.11) + (B.1.4), C1-4111: I.A + (H.1.5) +
(B.1.4), C1-4112: I.A + (H.1.7) + (B.1.4), C1-4113: I.A + (H.2.2) + (B.1.4), C1-4114: I.A + (H.2.3) +
(B.1.4), C1-4115: I.A + (H.2.5) + (B.1.4), C1-4116: I.A + (H.2.7) + (B.1.4), C1-4117: I.A +
(H.2.8) + (B.1.4), C1-4118: I.A + (H.3.2) + (B.1.4), C1-4119: I.A + (H.3.4) + (B.1.4), C1-4120: I.A +
(H.3.5) + (B.1.4), C1-4121: I.A + (H.4.9) + (B.1.4), C1-4122: I.A + (H.4.10) + (B.1.4), C1-4123:
I.A + (I.2.2) + (B.1.4), C1-4124: I.A + (I.2.5) + (B.1.4), C1-4125: I.A + (J.1.2) + (B.1.4), C1-4126:
I.A + (J.1.5) + (B.1.4), C1-4127: I.A + (J.1.8) + (B.1.4), C1-4128: I.A + (J.1.11) + (B.1.4), C1-
4129: I.A + (J.1.12) + (B.1.4), C1-4130: I.A + (K.1.3) + (B.1.4), C1-4131: I.A + (K.1.29) + (B.1.4),
C1-4132: I.A + (K.1.30) + (B.1.4), C1-4133: I.A + (K.1.31) + (B.1.4), C1-4134: I.A + (K.1.32) +
(B.1.4), C1-4135: I.A + (K.1.41) + (B.1.4), C1-4136: I.A + (K.1.42) + (B.1.4), C1-4137: I.A +
(K.1.44) + (B.1.4), C1-4138: I.A + (K.1.45) + (B.1.4), C1-4139: I.A + (K.1.47) + (B.1.4), C1-4140:
I.A + (K.1.49) + (B.1.4), C1-4141: I.A + (K.1.53) + (B.1.4), C1-4142: I.A + (K.1.54) + (B.1.4), C1-
4143: I.A + (A.1.4) + (B.1.5), C1-4144: I.A + (A.1.12) + (B.1.5), C1-4145: I.A + (A.1.21) +
(B.1.5), C1-4146: I.A + (A.1,21a) + (B.1.5), C1-4147: I.A + (A.1.24) + (B.1.5), C1-4148: I.A +
(A.1.26) + (B.1.5), C1-4149: I.A + (A.1.27) + (B.1.5), C1-4150: I.A + (A.1.30) + (B.1.5), C1-4151:
I.A + (A.1.31) + (B.1.5), C1-4152: I.A + (A.1.32) + (B.1.5), C1-4153: I.A + (A.1.35) + (B.1.5), C1-
4154: I.A + (A.2.1) + (B.1.5), C1-4155: I.A + (A.2.3) + (B.1.5), C1-4156: I.A + (A.2.5) + (B.1.5),
C1-4157: I.A + (A.3.11) + (B.1.5), C1-4158: I.A + (A.3.12) + (B.1.5), C1-4159: I.A + (A.3.15) +
(B.1.5), C1-4160: I.A + (A.3.16) + (B.1.5), C1-4161: I.A + (A.3.18) + (B.1.5), C1-4162: I.A +
(A.3.19) + (B.1.5), C1-4163: I.A + (A.3.20) + (B.1.5), C1-4164: I.A + (A.3.21) + (B.1.5), C1-4165:
I.A + (A.3.22) + (B.1.5), C1-4166: I.A + (A.3.23) + (B.1.5), C1-4167: I.A + (A.3.25) + (B.1.5), C1-
4168: I.A + (A.3.26) + (B.1.5), C1-4169: I.A + (A.3.27) + (B.1.5), C1-4170: I.A + (A.4.5) +
(B.1.5), C1-4171: I.A + (A.4.11) + (B.1.5), C1-4172: I.A + (B.1.8.) + (B.1.5), C1-4173: I.A +
(B.1.10) + (B.1.5), C1-4174: I.A + (B.1.11) + (B.1.5), C1-4175: I.A + (B.1.12) + (B.1.5), C1-4176:
I.A + (B.1.13) + (B.1.5), C1-4177: I.A + (B.1.17) + (B.1.5), C1-4178: I.A + (B.1.18) + (B.1.5), C1-
4179: I.A + (B.1.21) + (B.1.5), C1-4180: I.A + (B.1.22) + (B.1.5), C1-4181: I.A + (B.1.23) +
(B.1.5), C1-4182: I.A + (B.1.25) + (B.1.5), C1-4183: I.A + (B.1.26) + (B.1.5), C1-4184: I.A +
(B.1.29) + (B.1.5), C1-4185: I.A + (B.1.33) + (B.1.5), C1-4186: I.A + (B.1.34) + (B.1.5), C1-4187:
I.A + (B.1.37) + (B.1.5), C1-4188: I.A + (B.1.38) + (B.1.5), C1-4189: I.A + (B.1.43) + (B.1.5), C1-
4190: I.A + (B.1.46) + (B.1.5), C1-4191: I.A + (B.2.4) + (B.1.5), C1-4192: I.A + (B.2.5) + (B.1.5),
C1-4193: I.A + (B.2.6) + (B.1.5), C1-4194: I.A + (B.2.8) + (B.1.5), C1-4195: I.A + (C.1.1) +
(B.1.5), C1-4196: I.A + (C.1.2) + (B.1.5), C1-4197: I.A + (C.1.4) + (B.1.5), C1-4198: I.A + (C.1.5) +
(B.1.5), C1-4199: I.A + (C.2.6) + (B.1.5), C1-4200: I.A + (C.2.7) + (B.1.5), C1-4201: I.A +
(C.2.8) + (B.1.5), C1-4202: I.A + (D.1.1) + (B.1.5), C1-4203: I.A + (D.1.2) + (B.1.5), C1-4204: I.A +
(D.1.5) + (B.1.5), C1-4205: I.A + (D.2.4) + (B.1.5), C1-4206: I.A + (D.2.6) + (B.1.5), C1-4207:
I.A + (D.2.7) + (B.1.5), C1-4208: I.A + (E.1.1) + (B.1.5), C1-4209: I.A + (E.1.3) + (B.1.5), C1-
4210: I.A + (E.2.2) + (B.1.5), C1-4211: I.A + (E.2.3) + (B.1.5), C1-4212: I.A + (F.1.2) + (B.1.5),
C1-4213: I.A + (F.1.4) + (B.1.5), C1-4214: I.A + (F.1.5) + (B.1.5), C1-4215: I.A + (G.3.1) +
(B.1.5), C1-4216: I.A + (G.3.3) + (B.1.5), C1-4217: I.A + (G.3.6) + (B.1.5), C1-4218: I.A +
(G.5.1) + (B.1.5), C1-4219: I.A + (G.5.2) + (B.1.5), C1-4220: I.A + (G.5.3) + (B.1.5), C1-4221:
I.A + (G.5.4) + (B.1.5), C1-4222: I.A + (G.5.5) + (B.1.5), C1-4223: I.A + (G.5.6) + (B.1.5), C1-
4224: I.A + (G.5.7) + (B.1.5), C1-4225: I.A + (G.5.8) + (B.1.5), C1-4226: I.A + (G.5.9) + (B.1.5),
C1-4227: I.A + (G.5.10) + (B.1.5), C1-4228: I.A + (G.5.11) + (B.1.5), C1-4229: I.A + (H.1.5) +
(B.1.5), C1-4230: I.A + (H.1.7) + (B.1.5), C1-4231: I.A + (H.2.2) + (B.1.5), C1-4232: I.A + (H.2.3) +
(B.1.5), C1-4233: I.A + (H.2.5) + (B.1.5), C1-4234: I.A + (H.2.7) + (B.1.5), C1-4235: I.A +
(H.2.8) + (B.1.5), C1-4236: I.A + (H.3.2) + (B.1.5), C1-4237: I.A + (H.3.4) + (B.1.5), C1-4238: I.A +
(H.3.5) + (B.1.5), C1-4239: I.A + (H.4.9) + (B.1.5), C1-4240: I.A + (H.4.10) + (B.1.5), C1-4241:
I.A + (I.2.2) + (B.1.5), C1-4242: I.A + (I.2.5) + (B.1.5), C1-4243: I.A + (J.1.2) + (B.1.5), C1-4244:
I.A + (J.1.5) + (B.1.5), C1-4245: I.A + (J.1.8) + (B.1.5), C1-4246: I.A + (J.1.11) + (B.1.5), C1-
4247: I.A + (J.1.12) + (B.1.5), C1-4248: I.A + (K.1.3) + (B.1.5), C1-4249: I.A + (K.1.29) + (B.1.5),
C1-4250: I.A + (K.1.30) + (B.1.5), C1-4251: I.A + (K.1.31) + (B.1.5), C1-4252: I.A + (K.1.32) +
(B.1.5), C1-4253: I.A + (K.1.41) + (B.1.5), C1-4254: I.A + (K.1.42) + (B.1.5), C1-4255: I.A +
(K.1.44) + (B.1.5), C1-4256: I.A + (K.1.45) + (B.1.5), C1-4257: I.A + (K.1.47) + (B.1.5), C1-4258:
I.A + (K.1.49) + (B.1.5), C1-4259: I.A + (K.1.53) + (B.1.5), C1-4260: I.A + (K.1.54) + (B.1.5), C1-
4261: I.A + (A.1.4) + (B.1.8), C1-4262: I.A + (A.1.12) + (B.1.8), C1-4263: I.A + (A.1.21) +
(B.1.8), C1-4264: I.A + (A.1,21a) + (B.1.8), C1-4265: I.A + (A.1.24) + (B.1.8), C1-4266: I.A +
(A.1.26) + (B.1.8), C1-4267: I.A + (A.1.27) + (B.1.8), C1-4268: I.A + (A.1.30) + (B.1.8), C1-4269:
I.A + (A.1.31) + (B.1.8), C1-4270: I.A + (A.1.32) + (B.1.8), C1-4271: I.A + (A.1.35) + (B.1.8), C1-
4272: I.A + (A.2.1) + (B.1.8), C1-4273: I.A + (A.2.3) + (B.1.8), C1-4274: I.A + (A.2.5) + (B.1.8),
C1-4275: I.A + (A.3.11) + (B.1.8), C1-4276: I.A + (A.3.12) + (B.1.8), C1-4277: I.A + (A.3.15) +
(B.1.8), C1-4278: I.A + (A.3.16) + (B.1.8), C1-4279: I.A + (A.3.18) + (B.1.8), C1-4280: I.A +
(A.3.19) + (B.1.8), C1-4281: I.A + (A.3.20) + (B.1.8), C1-4282: I.A + (A.3.21) + (B.1.8), C1-4283:
I.A + (A.3.22) + (B.1.8), C1-4284: I.A + (A.3.23) + (B.1.8), C1-4285: I.A + (A.3.25) + (B.1.8), C1-
4286: I.A + (A.3.26) + (B.1.8), C1-4287: I.A + (A.3.27) + (B.1.8), C1-4288: I.A + (A.4.5) +
(B.1.8), C1-4289: I.A + (A.4.11) + (B.1.8), C1-4290: I.A + (B.1.10) + (B.1.8), C1-4291: I.A +
(B.1.11) + (B.1.8), C1-4292: I.A + (B.1.12) + (B.1.8), C1-4293: I.A + (B.1.13) + (B.1.8), C1-4294:
I.A + (B.1.17) + (B.1.8), C1-4295: I.A + (B.1.18) + (B.1.8), C1-4296: I.A + (B.1.21) + (B.1.8), C1-
4297: I.A + (B.1.22) + (B.1.8), C1-4298: I.A + (B.1.23) + (B.1.8), C1-4299: I.A + (B.1.25) +
(B.1.8), C1-4300: I.A + (B.1.26) + (B.1.8), C1-4301: I.A + (B.1.29) + (B.1.8), C1-4302: I.A +
(B.1.33) + (B.1.8), C1-4303: I.A + (B.1.34) + (B.1.8), C1-4304: I.A + (B.1.37) + (B.1.8), C1-4305:
I.A + (B.1.38) + (B.1.8), C1-4306: I.A + (B.1.43) + (B.1.8), C1-4307: I.A + (B.1.46) + (B.1.8), C1-
4308: I.A + (B.2.4) + (B.1.8), C1-4309: I.A + (B.2.5) + (B.1.8), C1-4310: I.A + (B.2.6) + (B.1.8),
C1-4311: I.A + (B.2.8) + (B.1.8), C1-4312: I.A + (C.1.1) + (B.1.8), C1-4313: I.A + (C.1.2) +
(B.1.8), C1-4314: I.A + (C.1.4) + (B.1.8), C1-4315: I.A + (C.1.5) + (B.1.8), C1-4316: I.A + (C.2.6) +
(B.1.8), C1-4317: I.A + (C.2.7) + (B.1.8), C1-4318: I.A + (C.2.8) + (B.1.8), C1-4319: I.A +
(D.1.1) + (B.1.8), C1-4320: I.A + (D.1.2) + (B.1.8), C1-4321: I.A + (D.1.5) + (B.1.8), C1-4322: I.A +
(D.2.4) + (B.1.8), C1-4323: I.A + (D.2.6) + (B.1.8), C1-4324: I.A + (D.2.7) + (B.1.8), C1-4325:
I.A + (E.1.1) + (B.1.8), C1-4326: I.A + (E.1.3) + (B.1.8), C1-4327: I.A + (E.2.2) + (B.1.8), C1-
4328: I.A + (E.2.3) + (B.1.8), C1-4329: I.A + (F.1.2) + (B.1.8), C1-4330: I.A + (F.1.4) + (B.1.8),
C1-4331: I.A + (F.1.5) + (B.1.8), C1-4332: I.A + (G.3.1) + (B.1.8), C1-4333: I.A + (G.3.3) +
(B.1.8), C1-4334: I.A + (G.3.6) + (B.1.8), C1-4335: I.A + (G.5.1) + (B.1.8), C1-4336: I.A +
(G.5.2) + (B.1.8), C1-4337: I.A + (G.5.3) + (B.1.8), C1-4338: I.A + (G.5.4) + (B.1.8), C1-4339:
I.A + (G.5.5) + (B.1.8), C1-4340: I.A + (G.5.6) + (B.1.8), C1-4341: I.A + (G.5.7) + (B.1.8), C1-
4342: I.A + (G.5.8) + (B.1.8), C1-4343: I.A + (G.5.9) + (B.1.8), C1-4344: I.A + (G.5.10) + (B.1.8),
C1-4345: I.A + (G.5.11) + (B.1.8), C1-4346: I.A + (H.1.5) + (B.1.8), C1-4347: I.A + (H.1.7) +
(B.1.8), C1-4348: I.A + (H.2.2) + (B.1.8), C1-4349: I.A + (H.2.3) + (B.1.8), C1-4350: I.A + (H.2.5) +
(B.1.8), C1-4351: I.A + (H.2.7) + (B.1.8), C1-4352: I.A + (H.2.8) + (B.1.8), C1-4353: I.A +
(H.3.2) + (B.1.8), C1-4354: I.A + (H.3.4) + (B.1.8), C1-4355: I.A + (H.3.5) + (B.1.8), C1-4356: I.A +
(H.4.9) + (B.1.8), C1-4357: I.A + (H.4.10) + (B.1.8), C1-4358: I.A + (I.2.2) + (B.1.8), C1-4359:
I.A + (I.2.5) + (B.1.8), C1-4360: I.A + (J.1.2) + (B.1.8), C1-4361: I.A + (J.1.5) + (B.1.8), C1-4362:
I.A + (J.1.8) + (B.1.8), C1-4363: I.A + (J.1.11) + (B.1.8), C1-4364: I.A + (J.1.12) + (B.1.8), C1-
4365: I.A + (K.1.3) + (B.1.8), C1-4366: I.A + (K.1.29) + (B.1.8), C1-4367: I.A + (K.1.30) +
(B.1.8), C1-4368: I.A + (K.1.31) + (B.1.8), C1-4369: I.A + (K.1.32) + (B.1.8), C1-4370: I.A +
(K.1.41) + (B.1.8), C1-4371: I.A + (K.1.42) + (B.1.8), C1-4372: I.A + (K.1.44) + (B.1.8), C1-4373:
I.A + (K.1.45) + (B.1.8), C1-4374: I.A + (K.1.47) + (B.1.8), C1-4375: I.A + (K.1.49) + (B.1.8), C1-
4376: I.A + (K.1.53) + (B.1.8), C1-4377: I.A + (K.1.54) + (B.1.8), C1-4378: I.A + (A.1.4) +
(B.1.17), C1-4379: I.A + (A.1.12) + (B.1.17), C1-4380: I.A + (A.1.21) + (B.1.17), C1-4381: I.A +
(A.1.21a) + (B.1.17), C1-4382: I.A + (A.1.24) + (B.1.17), C1-4383: I.A + (A.1.26) + (B.1.17), C1-
4384: I.A + (A.1.27) + (B.1.17), C1-4385: I.A + (A.1.30) + (B.1.17), C1-4386: I.A + (A.1.31) +
(B.1.17), C1-4387: I.A + (A.1.32) + (B.1.17), C1-4388: I.A + (A.1.35) + (B.1.17), C1-4389: I.A +
(A.2.1) + (B.1.17), C1-4390: I.A + (A.2.3) + (B.1.17), C1-4391: I.A + (A.2.5) + (B.1.17), C1-4392:
I.A + (A.3.11) + (B.1.17), C1-4393: I.A + (A.3.12) + (B.1.17), C1-4394: I.A + (A.3.15) + (B.1.17),
C1-4395: I.A + (A.3.16) + (B.1.17), C1-4396: I.A + (A.3.18) + (B.1.17), C1-4397: I.A + (A.3.19) +
(B.1.17), C1-4398: I.A + (A.3.20) + (B.1.17), C1-4399: I.A + (A.3.21) + (B.1.17), C1-4400: I.A +
(A.3.22) + (B.1.17), C1-4401: I.A + (A.3.23) + (B.1.17), C1-4402: I.A + (A.3.25) + (B.1.17), C1-
4403: I.A + (A.3.26) + (B.1.17), C1-4404: I.A + (A.3.27) + (B.1.17), C1-4405: I.A + (A.4.5) +
(B.1.17), C1-4406: I.A + (A.4.11) + (B.1.17), C1-4407: I.A + (B.1.10) + (B.1.17), C1-4408: I.A +
(B.1.11) + (B.1.17), C1-4409: I.A + (B.1.12) + (B.1.17), C1-4410: I.A + (B.1.13) + (B.1.17), C1-
4411: I.A + (B.1.18) + (B.1.17), C1-4412: I.A + (B.1.21) + (B.1.17), C1-4413: I.A + (B.1.22) +
(B.1.17), C1-4414: I.A + (B.1.23) + (B.1.17), C1-4415: I.A + (B.1.25) + (B.1.17), C1-4416: I.A +
(B.1.26) + (B.1.17), C1-4417: I.A + (B.1.29) + (B.1.17), C1-4418: I.A + (B.1.33) + (B.1.17), C1-
4419: I.A + (B.1.34) + (B.1.17), C1-4420: I.A + (B.1.37) + (B.1.17), C1-4421: I.A + (B.1.38) +
(B.1.17), C1-4422: I.A + (B.1.43) + (B.1.17), C1-4423: I.A + (B.1.46) + (B.1.17), C1-4424: I.A +
(B.2.4) + (B.1.17), C1-4425: I.A + (B.2.5) + (B.1.17), C1-4426: I.A + (B.2.6) + (B.1.17), C1-4427:
I.A + (B.2.8) + (B.1.17), C1-4428: I.A + (C.1.1) + (B.1.17), C1-4429: I.A + (C.1.2) + (B.1.17), C1-
4430: I.A + (C.1.4) + (B.1.17), C1-4431: I.A + (C.1.5) + (B.1.17), C1-4432: I.A + (C.2.6) +
(B.1.17), C1-4433: I.A + (C.2.7) + (B.1.17), C1-4434: I.A + (C.2.8) + (B.1.17), C1-4435: I.A +
(D.1.1) + (B.1.17), C1-4436: I.A + (D.1.2) + (B.1.17), C1-4437: I.A + (D.1.5) + (B.1.17), C1-
4438: I.A + (D.2.4) + (B.1.17), C1-4439: I.A + (D.2.6) + (B.1.17), C1-4440: I.A + (D.2.7) +
(B.1.17), C1-4441: I.A + (E.1.1) + (B.1.17), C1-4442: I.A + (E.1.3) + (B.1.17), C1-4443: I.A +
(E.2.2) + (B.1.17), C1-4444: I.A + (E.2.3) + (B.1.17), C1-4445: I.A + (F.1.2) + (B.1.17), C1-4446:
I.A + (F.1.4) + (B.1.17), C1-4447: I.A + (F.1.5) + (B.1.17), C1-4448: I.A + (G.3.1) + (B.1.17), C1-
4449: I.A + (G.3.3) + (B.1.17), C1-4450: I.A + (G.3.6) + (B.1.17), C1-4451: I.A + (G.5.1) +
(B.1.17), C1-4452: I.A + (G.5.2) + (B.1.17), C1-4453: I.A + (G.5.3) + (B.1.17), C1-4454: I.A +
(G.5.4) + (B.1.17), C1-4455: I.A + (G.5.5) + (B.1.17), C1-4456: I.A + (G.5.6) + (B.1.17), C1-
4457: I.A + (G.5.7) + (B.1.17), C1-4458: I.A + (G.5.8) + (B.1.17), C1-4459: I.A + (G.5.9) +
(B.1.17), C1-4460: I.A + (G.5.10) + (B.1.17), C1-4461: I.A + (G.5.11) + (B.1.17), C1-4462: I.A +
(H.1.5) + (B.1.17), C1-4463: I.A + (H.1.7) + (B.1.17), C1-4464: I.A + (H.2.2) + (B.1.17), C1-
4465: I.A + (H.2.3) + (B.1.17), C1-4466: I.A + (H.2.5) + (B.1.17), C1-4467: I.A + (H.2.7) +
(B.1.17), C1-4468: I.A + (H.2.8) + (B.1.17), C1-4469: I.A + (H.3.2) + (B.1.17), C1-4470: I.A +
(H.3.4) + (B.1.17), C1-4471: I.A + (H.3.5) + (B.1.17), C1-4472: I.A + (H.4.9) + (B.1.17), C1-
4473: I.A + (H.4.10) + (B.1.17), C1-4474: I.A + (I.2.2) + (B.1.17), C1-4475: I.A + (I.2.5) +
(B.1.17), C1-4476: I.A + (J.1.2) + (B.1.17), C1-4477: I.A + (J.1.5) + (B.1.17), C1-4478: I.A +
(J.1.8) + (B.1.17), C1-4479: I.A + (J.1.11) + (B.1.17), C1-4480: I.A + (J.1.12) + (B.1.17), C1-
4481: I.A + (K.1.3) + (B.1.17), C1-4482: I.A + (K.1.29) + (B.1.17), C1-4483: I.A + (K.1.30) +
(B.1.17), C1-4484: I.A + (K.1.31) + (B.1.17), C1-4485: I.A + (K.1.32) + (B.1.17), C1-4486: I.A +
(K.1.41) + (B.1.17), C1-4487: I.A + (K.1.42) + (B.1.17), C1-4488: I.A + (K.1.44) + (B.1.17), C1-
4489: I.A + (K.1.45) + (B.1.17), C1-4490: I.A + (K.1.47) + (B.1.17), C1-4491: I.A + (K.1.49) +
(B.1.17), C1-4492: I.A + (K.1.53) + (B.1.17), C1-4493: I.A + (K.1.54) + (B.1.17), C1-4494: I.A +
(A.1.4) + (B.1.22), C1-4495: I.A + (A.1.12) + (B.1.22), C1-4496: I.A + (A.1.21) + (B.1.22), C1-
4497: I.A + (A.1.21a) + (B.1.22), C1-4498: I.A + (A.1.24) + (B.1.22), C1-4499: I.A + (A.1.26) +
(B.1.22), C1-4500: I.A + (A.1.27) + (B.1.22), C1-4501: I.A + (A.1.30) + (B.1.22), C1-4502: I.A +
(A.1.31) + (B.1.22), C1-4503: I.A + (A.1.32) + (B.1.22), C1-4504: I.A + (A.1.35) + (B.1.22), C1-
4505: I.A + (A.2.1) + (B.1.22), C1-4506: I.A + (A.2.3) + (B.1.22), C1-4507: I.A + (A.2.5) +
(B.1.22), C1-4508: I.A + (A.3.11) + (B.1.22), C1-4509: I.A + (A.3.12) + (B.1.22), C1-4510: I.A +
(A.3.15) + (B.1.22), C1-4511: I.A + (A.3.16) + (B.1.22), C1-4512: I.A + (A.3.18) + (B.1.22), C1-
4513: I.A + (A.3.19) + (B.1.22), C1-4514: I.A + (A.3.20) + (B.1.22), C1-4515: I.A + (A.3.21) +
(B.1.22), C1-4516: I.A + (A.3.22) + (B.1.22), C1-4517: I.A + (A.3.23) + (B.1.22), C1-4518: I.A +
(A.3.25) + (B.1.22), C1-4519: I.A + (A.3.26) + (B.1.22), C1-4520: I.A + (A.3.27) + (B.1.22), C1-
4521: I.A + (A.4.5) + (B.1.22), C1-4522: I.A + (A.4.11) + (B.1.22), C1-4523: I.A + (B.1.10) +
(B.1.22), C1-4524: I.A + (B.1.11) + (B.1.22), C1-4525: I.A + (B.1.12) + (B.1.22), C1-4526: I.A +
(B.1.13) + (B.1.22), C1-4527: I.A + (B.1.18) + (B.1.22), C1-4528: I.A + (B.1.21) + (B.1.22), C1-
4529: I.A + (B.1.23) + (B.1.22), C1-4530: I.A + (B.1.25) + (B.1.22), C1-4531: I.A + (B.1.26) +
(B.1.22), C1-4532: I.A + (B.1.29) + (B.1.22), C1-4533: I.A + (B.1.33) + (B.1.22), C1-4534: I.A +
(B.1.34) + (B.1.22), C1-4535: I.A + (B.1.37) + (B.1.22), C1-4536: I.A + (B.1.38) + (B.1.22), C1-
4537: I.A + (B.1.43) + (B.1.22), C1-4538: I.A + (B.1.46) + (B.1.22), C1-4539: I.A + (B.2.4) +
(B.1.22), C1-4540: I.A + (B.2.5) + (B.1.22), C1-4541: I.A + (B.2.6) + (B.1.22), C1-4542: I.A +
(B.2.8) + (B.1.22), C1-4543: I.A + (C.1.1) + (B.1.22), C1-4544: I.A + (C.1.2) + (B.1.22), C1-4545:
I.A + (C.1.4) + (B.1.22), C1-4546: I.A + (C.1.5) + (B.1.22), C1-4547: I.A + (C.2.6) + (B.1.22), C1-
4548: I.A + (C.2.7) + (B.1.22), C1-4549: I.A + (C.2.8) + (B.1.22), C1-4550: I.A + (D.1.1) +
(B.1.22), C1-4551: I.A + (D.1.2) + (B.1.22), C1-4552: I.A + (D.1.5) + (B.1.22), C1-4553: I.A +
(D.2.4) + (B.1.22), C1-4554: I.A + (D.2.6) + (B.1.22), C1-4555: I.A + (D.2.7) + (B.1.22), C1-
4556: I.A + (E.1.1) + (B.1.22), C1-4557: I.A + (E.1.3) + (B.1.22), C1-4558: I.A + (E.2.2) +
(B.1.22), C1-4559: I.A + (E.2.3) + (B.1.22), C1-4560: I.A + (F.1.2) + (B.1.22), C1-4561: I.A +
(F.1.4) + (B.1.22), C1-4562: I.A + (F.1.5) + (B.1.22), C1-4563: I.A + (G.3.1) + (B.1.22), C1-4564:
I.A + (G.3.3) + (B.1.22), C1-4565: I.A + (G.3.6) + (B.1.22), C1-4566: I.A + (G.5.1) + (B.1.22),
C1-4567: I.A + (G.5.2) + (B.1.22), C1-4568: I.A + (G.5.3) + (B.1.22), C1-4569: I.A + (G.5.4) +
(B.1.22), C1-4570: I.A + (G.5.5) + (B.1.22), C1-4571: I.A + (G.5.6) + (B.1.22), C1-4572: I.A +
(G.5.7) + (B.1.22), C1-4573: I.A + (G.5.8) + (B.1.22), C1-4574: I.A + (G.5.9) + (B.1.22), C1-
4575: I.A + (G.5.10) + (B.1.22), C1-4576: I.A + (G.5.11) + (B.1.22), C1-4577: I.A + (H.1.5) +
(B.1.22), C1-4578: I.A + (H.1.7) + (B.1.22), C1-4579: I.A + (H.2.2) + (B.1.22), C1-4580: I.A +
(H.2.3) + (B.1.22), C1-4581: I.A + (H.2.5) + (B.1.22), C1-4582: I.A + (H.2.7) + (B.1.22), C1-
4583: I.A + (H.2.8) + (B.1.22), C1-4584: I.A + (H.3.2) + (B.1.22), C1-4585: I.A + (H.3.4) +
(B.1.22), C1-4586: I.A + (H.3.5) + (B.1.22), C1-4587: I.A + (H.4.9) + (B.1.22), C1-4588: I.A +
(H.4.10) + (B.1.22), C1-4589: I.A + (I.2.2) + (B.1.22), C1-4590: I.A + (I.2.5) + (B.1.22), C1-4591:
I.A + (J.1.2) + (B.1.22), C1-4592: I.A + (J.1.5) + (B.1.22), C1-4593: I.A + (J.1.8) + (B.1.22), C1-
4594: I.A + (J.1.11) + (B.1.22), C1-4595: I.A + (J.1.12) + (B.1.22), C1-4596: I.A + (K.1.3) +
(B.1.22), C1-4597: I.A + (K.1.29) + (B.1.22), C1-4598: I.A + (K.1.30) + (B.1.22), C1-4599: I.A +
(K.1.31) + (B.1.22), C1-4600: I.A + (K.1.32) + (B.1.22), C1-4601: I.A + (K.1.41) + (B.1.22), C1-
4602: I.A + (K.1.42) + (B.1.22), C1-4603: I.A + (K.1.44) + (B.1.22), C1-4604: I.A + (K.1.45) +
(B.1.22), C1-4605: I.A + (K.1.47) + (B.1.22), C1-4606: I.A + (K.1.49) + (B.1.22), C1-4607: I.A +
(K.1.53) + (B.1.22), C1-4608: I.A + (K.1.54) + (B.1.22), C1-4609: I.A + (A.1.4) + (B.1.23), C1-
4610: I.A + (A.1.12) + (B.1.23), C1-4611: I.A + (A.1.21) + (B.1.23), C1-4612: I.A + (A.1,21a) +
(B.1.23), C1-4613: I.A + (A.1.24) + (B.1.23), C1-4614: I.A + (A.1.26) + (B.1.23), C1-4615: I.A +
(A.1.27) + (B.1.23), C1-4616: I.A + (A.1.30) + (B.1.23), C1-4617: I.A + (A.1.31) + (B.1.23), C1-
4618: I.A + (A.1.32) + (B.1.23), C1-4619: I.A + (A.1.35) + (B.1.23), C1-4620: I.A + (A.2.1) +
(B.1.23), C1-4621: I.A + (A.2.3) + (B.1.23), C1-4622: I.A + (A.2.5) + (B.1.23), C1-4623: I.A +
(A.3.11) + (B.1.23), C1-4624: I.A + (A.3.12) + (B.1.23), C1-4625: I.A + (A.3.15) + (B.1.23), C1-
4626: I.A + (A.3.16) + (B.1.23), C1-4627: I.A + (A.3.18) + (B.1.23), C1-4628: I.A + (A.3.19) +
(B.1.23), C1-4629: I.A + (A.3.20) + (B.1.23), C1-4630: I.A + (A.3.21) + (B.1.23), C1-4631: I.A +
(A.3.22) + (B.1.23), C1-4632: I.A + (A.3.23) + (B.1.23), C1-4633: I.A + (A.3.25) + (B.1.23), C1-
4634: I.A + (A.3.26) + (B.1.23), C1-4635: I.A + (A.3.27) + (B.1.23), C1-4636: I.A + (A.4.5) +
(B.1.23), C1-4637: I.A + (A.4.11) + (B.1.23), C1-4638: I.A + (B.1.10) + (B.1.23), C1-4639: I.A +
(B.1.11) + (B.1.23), C1-4640: I.A + (B.1.12) + (B.1.23), C1-4641: I.A + (B.1.13) + (B.1.23), C1-
4642: I.A + (B.1.18) + (B.1.23), C1-4643: I.A + (B.1.21) + (B.1.23), C1-4644: I.A + (B.1.25) +
(B.1.23), C1-4645: I.A + (B.1.26) + (B.1.23), C1-4646: I.A + (B.1.29) + (B.1.23), C1-4647: I.A +
(B.1.33) + (B.1.23), C1-4648: I.A + (B.1.34) + (B.1.23), C1-4649: I.A + (B.1.37) + (B.1.23), C1-
4650: I.A + (B.1.38) + (B.1.23), C1-4651: I.A + (B.1.43) + (B.1.23), C1-4652: I.A + (B.1.46) +
(B.1.23), C1-4653: I.A + (B.2.4) + (B.1.23), C1-4654: I.A + (B.2.5) + (B.1.23), C1-4655: I.A +
(B.2.6) + (B.1.23), C1-4656: I.A + (B.2.8) + (B.1.23), C1-4657: I.A + (C.1.1) + (B.1.23), C1-4658:
I.A + (C.1.2) + (B.1.23), C1-4659: I.A + (C.1.4) + (B.1.23), C1-4660: I.A + (C.1.5) + (B.1.23), C1-
4661: I.A + (C.2.6) + (B.1.23), C1-4662: I.A + (C.2.7) + (B.1.23), C1-4663: I.A + (C.2.8) +
(B.1.23), C1-4664: I.A + (D.1.1) + (B.1.23), C1-4665: I.A + (D.1.2) + (B.1.23), C1-4666: I.A +
(D.1.5) + (B.1.23), C1-4667: I.A + (D.2.4) + (B.1.23), C1-4668: I.A + (D.2.6) + (B.1.23), C1-
4669: I.A + (D.2.7) + (B.1.23), C1-4670: I.A + (E.1.1) + (B.1.23), C1-4671: I.A + (E.1.3) +
(B.1.23), C1-4672: I.A + (E.2.2) + (B.1.23), C1-4673: I.A + (E.2.3) + (B.1.23), C1-4674: I.A +
(F.1.2) + (B.1.23), C1-4675: I.A + (F.1.4) + (B.1.23), C1-4676: I.A + (F.1.5) + (B.1.23), C1-4677:
I.A + (G.3.1) + (B.1.23), C1-4678: I.A + (G.3.3) + (B.1.23), C1-4679: I.A + (G.3.6) + (B.1.23),
C1-4680: I.A + (G.5.1) + (B.1.23), C1-4681: I.A + (G.5.2) + (B.1.23), C1-4682: I.A + (G.5.3) +
(B.1.23), C1-4683: I.A + (G.5.4) + (B.1.23), C1-4684: I.A + (G.5.5) + (B.1.23), C1-4685: I.A +
(G.5.6) + (B.1.23), C1-4686: I.A + (G.5.7) + (B.1.23), C1-4687: I.A + (G.5.8) + (B.1.23), C1-
4688: I.A + (G.5.9) + (B.1.23), C1-4689: I.A + (G.5.10) + (B.1.23), C1-4690: I.A + (G.5.11) +
(B.1.23), C1-4691: I.A + (H.1.5) + (B.1.23), C1-4692: I.A + (H.1.7) + (B.1.23), C1-4693: I.A +
(H.2.2) + (B.1.23), C1-4694: I.A + (H.2.3) + (B.1.23), C1-4695: I.A + (H.2.5) + (B.1.23), C1-
4696: I.A + (H.2.7) + (B.1.23), C1-4697: I.A + (H.2.8) + (B.1.23), C1-4698: I.A + (H.3.2) +
(B.1.23), C1-4699: I.A + (H.3.4) + (B.1.23), C1-4700: I.A + (H.3.5) + (B.1.23), C1-4701: I.A +
(H.4.9) + (B.1.23), C1-4702: I.A + (H.4.10) + (B.1.23), C1-4703: I.A + (I.2.2) + (B.1.23), C1-
4704: I.A + (I.2.5) + (B.1.23), C1-4705: I.A + (J.1.2) + (B.1.23), C1-4706: I.A + (J.1.5) + (B.1.23),
C1-4707: I.A + (J.1.8) + (B.1.23), C1-4708: I.A + (J.1.11) + (B.1.23), C1-4709: I.A + (J.1.12) +
(B.1.23), C1-4710: I.A + (K.1.3) + (B.1.23), C1-4711: I.A + (K.1.29) + (B.1.23), C1-4712: I.A +
(K.1.30) + (B.1.23), C1-4713: I.A + (K.1.31) + (B.1.23), C1-4714: I.A + (K.1.32) + (B.1.23), C1-
4715: I.A + (K.1.41) + (B.1.23), C1-4716: I.A + (K.1.42) + (B.1.23), C1-4717: I.A + (K.1.44) +
(B.1.23), C1-4718: I.A + (K.1.45) + (B.1.23), C1-4719: I.A + (K.1.47) + (B.1.23), C1-4720: I.A +
(K.1.49) + (B.1.23), C1-4721: I.A + (K.1.53) + (B.1.23), C1-4722: I.A + (K.1.54) + (B.1.23), C1-
4723: I.A + (A.1.4) + (B.1.25), C1-4724: I.A + (A.1.12) + (B.1.25), C1-4725: I.A + (A.1.21) +
(B.1.25), C1-4726: I.A + (A.1,21a) + (B.1.25), C1-4727: I.A + (A.1.24) + (B.1.25), C1-4728: I.A +
(A.1.26) + (B.1.25), C1-4729: I.A + (A.1.27) + (B.1.25), C1-4730: I.A + (A.1.30) + (B.1.25), C1-
4731: I.A + (A.1.31) + (B.1.25), C1-4732: I.A + (A.1.32) + (B.1.25), C1-4733: I.A + (A.1.35) +
(B.1.25), C1-4734: I.A + (A.2.1) + (B.1.25), C1-4735: I.A + (A.2.3) + (B.1.25), C1-4736: I.A +
(A.2.5) + (B.1.25), C1-4737: I.A + (A.3.11) + (B.1.25), C1-4738: I.A + (A.3.12) + (B.1.25), C1-
4739: I.A + (A.3.15) + (B.1.25), C1-4740: I.A + (A.3.16) + (B.1.25), C1-4741: I.A + (A.3.18) +
(B.1.25), C1-4742: I.A + (A.3.19) + (B.1.25), C1-4743: I.A + (A.3.20) + (B.1.25), C1-4744: I.A +
(A.3.21) + (B.1.25), C1-4745: I.A + (A.3.22) + (B.1.25), C1-4746: I.A + (A.3.23) + (B.1.25), C1-
4747: I.A + (A.3.25) + (B.1.25), C1-4748: I.A + (A.3.26) + (B.1.25), C1-4749: I.A + (A.3.27) +
(B.1.25), C1-4750: I.A + (A.4.5) + (B.1.25), C1-4751: I.A + (A.4.11) + (B.1.25), C1-4752: I.A +
(B.1.10) + (B.1.25), C1-4753: I.A + (B.1.11) + (B.1.25), C1-4754: I.A + (B.1.12) + (B.1.25), C1-
4755: I.A + (B.1.13) + (B.1.25), C1-4756: I.A + (B.1.18) + (B.1.25), C1-4757: I.A + (B.1.21) +
(B.1.25), C1-4758: I.A + (B.1.26) + (B.1.25), C1-4759: I.A + (B.1.29) + (B.1.25), C1-4760: I.A +
(B.1.33) + (B.1.25), C1-4761: I.A + (B.1.34) + (B.1.25), C1-4762: I.A + (B.1.37) + (B.1.25), C1-
4763: I.A + (B.1.38) + (B.1.25), C1-4764: I.A + (B.1.43) + (B.1.25), C1-4765: I.A + (B.1.46) +
(B.1.25), C1-4766: I.A + (B.2.4) + (B.1.25), C1-4767: I.A + (B.2.5) + (B.1.25), C1-4768: I.A +
(B.2.6) + (B.1.25), C1-4769: I.A + (B.2.8) + (B.1.25), C1-4770: I.A + (C.1.1) + (B.1.25), C1-4771:
I.A + (C.1.2) + (B.1.25), C1-4772: I.A + (C.1.4) + (B.1.25), C1-4773: I.A + (C.1.5) + (B.1.25), C1-
4774: I.A + (C.2.6) + (B.1.25), C1-4775: I.A + (C.2.7) + (B.1.25), C1-4776: I.A + (C.2.8) +
(B.1.25), C1-4777: I.A + (D.1.1) + (B.1.25), C1-4778: I.A + (D.1.2) + (B.1.25), C1-4779: I.A +
(D.1.5) + (B.1.25), C1-4780: I.A + (D.2.4) + (B.1.25), C1-4781: I.A + (D.2.6) + (B.1.25), C1-
4782: I.A + (D.2.7) + (B.1.25), C1-4783: I.A + (E.1.1) + (B.1.25), C1-4784: I.A + (E.1.3) +
(B.1.25), C1-4785: I.A + (E.2.2) + (B.1.25), C1-4786: I.A + (E.2.3) + (B.1.25), C1-4787: I.A +
(F.1.2) + (B.1.25), C1-4788: I.A + (F.1.4) + (B.1.25), C1-4789: I.A + (F.1.5) + (B.1.25), C1-4790:
I.A + (G.3.1) + (B.1.25), C1-4791: I.A + (G.3.3) + (B.1.25), C1-4792: I.A + (G.3.6) + (B.1.25),
C1-4793: I.A + (G.5.1) + (B.1.25), C1-4794: I.A + (G.5.2) + (B.1.25), C1-4795: I.A + (G.5.3) +
(B.1.25), C1-4796: I.A + (G.5.4) + (B.1.25), C1-4797: I.A + (G.5.5) + (B.1.25), C1-4798: I.A +
(G.5.6) + (B.1.25), C1-4799: I.A + (G.5.7) + (B.1.25), C1-4800: I.A + (G.5.8) + (B.1.25), C1-
4801: I.A + (G.5.9) + (B.1.25), C1-4802: I.A + (G.5.10) + (B.1.25), C1-4803: I.A + (G.5.11) +
(B.1.25), C1-4804: I.A + (H.1.5) + (B.1.25), C1-4805: I.A + (H.1.7) + (B.1.25), C1-4806: I.A +
(H.2.2) + (B.1.25), C1-4807: I.A + (H.2.3) + (B.1.25), C1-4808: I.A + (H.2.5) + (B.1.25), C1-
4809: I.A + (H.2.7) + (B.1.25), C1-4810: I.A + (H.2.8) + (B.1.25), C1-4811: I.A + (H.3.2) +
(B.1.25), C1-4812: I.A + (H.3.4) + (B.1.25), C1-4813: I.A + (H.3.5) + (B.1.25), C1-4814: I.A +
(H.4.9) + (B.1.25), C1-4815: I.A + (H.4.10) + (B.1.25), C1-4816: I.A + (I.2.2) + (B.1.25), C1-
4817: I.A + (I.2.5) + (B.1.25), C1-4818: I.A + (J.1.2) + (B.1.25), C1-4819: I.A + (J.1.5) + (B.1.25),
C1-4820: I.A + (J.1.8) + (B.1.25), C1-4821: I.A + (J.1.11) + (B.1.25), C1-4822: I.A + (J.1.12) +
(B.1.25), C1-4823: I.A + (K.1.3) + (B.1.25), C1-4824: I.A + (K.1.29) + (B.1.25), C1-4825: I.A +
(K.1.30) + (B.1.25), C1-4826: I.A + (K.1.31) + (B.1.25), C1-4827: I.A + (K.1.32) + (B.1.25), C1-
4828: I.A + (K.1.41) + (B.1.25), C1-4829: I.A + (K.1.42) + (B.1.25), C1-4830: I.A + (K.1.44) +
(B.1.25), C1-4831: I.A + (K.1.45) + (B.1.25), C1-4832: I.A + (K.1.47) + (B.1.25), C1-4833: I.A +
(K.1.49) + (B.1.25), C1-4834: I.A + (K.1.53) + (B.1.25), C1-4835: I.A + (K.1.54) + (B.1.25), C1-
4836: I.A + (A.1.4) + (B.1.29), C1-4837: I.A + (A.1.12) + (B.1.29), C1-4838: I.A + (A.1.21) +
(B.1.29), C1-4839: I.A + (A.1,21a) + (B.1.29), C1-4840: I.A + (A.1.24) + (B.1.29), C1-4841: I.A +
(A.1.26) + (B.1.29), C1-4842: I.A + (A.1.27) + (B.1.29), C1-4843: I.A + (A.1.30) + (B.1.29), C1-
4844: I.A + (A.1.31) + (B.1.29), C1-4845: I.A + (A.1.32) + (B.1.29), C1-4846: I.A + (A.1.35) +
(B.1.29), C1-4847: I.A + (A.2.1) + (B.1.29), C1-4848: I.A + (A.2.3) + (B.1.29), C1-4849: I.A +
(A.2.5) + (B.1.29), C1-4850: I.A + (A.3.11) + (B.1.29), C1-4851: I.A + (A.3.12) + (B.1.29), C1-
4852: I.A + (A.3.15) + (B.1.29), C1-4853: I.A + (A.3.16) + (B.1.29), C1-4854: I.A + (A.3.18) +
(B.1.29), C1-4855: I.A + (A.3.19) + (B.1.29), C1-4856: I.A + (A.3.20) + (B.1.29), C1-4857: I.A +
(A.3.21) + (B.1.29), C1-4858: I.A + (A.3.22) + (B.1.29), C1-4859: I.A + (A.3.23) + (B.1.29), C1-
4860: I.A + (A.3.25) + (B.1.29), C1-4861: I.A + (A.3.26) + (B.1.29), C1-4862: I.A + (A.3.27) +
(B.1.29), C1-4863: I.A + (A.4.5) + (B.1.29), C1-4864: I.A + (A.4.11) + (B.1.29), C1-4865: I.A +
(B.1.10) + (B.1.29), C1-4866: I.A + (B.1.11) + (B.1.29), C1-4867: I.A + (B.1.12) + (B.1.29), C1-
4868: I.A + (B.1.13) + (B.1.29), C1-4869: I.A + (B.1.18) + (B.1.29), C1-4870: I.A + (B.1.21) +
(B.1.29), C1-4871: I.A + (B.1.26) + (B.1.29), C1-4872: I.A + (B.1.33) + (B.1.29), C1-4873: I.A +
(B.1.34) + (B.1.29), C1-4874: I.A + (B.1.37) + (B.1.29), C1-4875: I.A + (B.1.38) + (B.1.29), C1-
4876: I.A + (B.1.43) + (B.1.29), C1-4877: I.A + (B.1.46) + (B.1.29), C1-4878: I.A + (B.2.4) +
(B.1.29), C1-4879: I.A + (B.2.5) + (B.1.29), C1-4880: I.A + (B.2.6) + (B.1.29), C1-4881: I.A +
(B.2.8) + (B.1.29), C1-4882: I.A + (C.1.1) + (B.1.29), C1-4883: I.A + (C.1.2) + (B.1.29), C1-4884:
I.A + (C.1.4) + (B.1.29), C1-4885: I.A + (C.1.5) + (B.1.29), C1-4886: I.A + (C.2.6) + (B.1.29), C1-
4887: I.A + (C.2.7) + (B.1.29), C1-4888: I.A + (C.2.8) + (B.1.29), C1-4889: I.A + (D.1.1) +
(B.1.29), C1-4890: I.A + (D.1.2) + (B.1.29), C1-4891: I.A + (D.1.5) + (B.1.29), C1-4892: I.A +
(D.2.4) + (B.1.29), C1-4893: I.A + (D.2.6) + (B.1.29), C1-4894: I.A + (D.2.7) + (B.1.29), C1-
4895: I.A + (E.1.1) + (B.1.29), C1-4896: I.A + (E.1.3) + (B.1.29), C1-4897: I.A + (E.2.2) +
(B.1.29), C1-4898: I.A + (E.2.3) + (B.1.29), C1-4899: I.A + (F.1.2) + (B.1.29), C1-4900: I.A +
(F.1.4) + (B.1.29), C1-4901: I.A + (F.1.5) + (B.1.29), C1-4902: I.A + (G.3.1) + (B.1.29), C1-4903:
I.A + (G.3.3) + (B.1.29), C1-4904: I.A + (G.3.6) + (B.1.29), C1-4905: I.A + (G.5.1) + (B.1.29),
C1-4906: I.A + (G.5.2) + (B.1.29), C1-4907: I.A + (G.5.3) + (B.1.29), C1-4908: I.A + (G.5.4) +
(B.1.29), C1-4909: I.A + (G.5.5) + (B.1.29), C1-4910: I.A + (G.5.6) + (B.1.29), C1-4911: I.A +
(G.5.7) + (B.1.29), C1-4912: I.A + (G.5.8) + (B.1.29), C1-4913: I.A + (G.5.9) + (B.1.29), C1-
4914: I.A + (G.5.10) + (B.1.29), C1-4915: I.A + (G.5.11) + (B.1.29), C1-4916: I.A + (H.1.5) +
(B.1.29), C1-4917: I.A + (H.1.7) + (B.1.29), C1-4918: I.A + (H.2.2) + (B.1.29), C1-4919: I.A +
(H.2.3) + (B.1.29), C1-4920: I.A + (H.2.5) + (B.1.29), C1-4921: I.A + (H.2.7) + (B.1.29), C1-
4922: I.A + (H.2.8) + (B.1.29), C1-4923: I.A + (H.3.2) + (B.1.29), C1-4924: I.A + (H.3.4) +
(B.1.29), C1-4925: I.A + (H.3.5) + (B.1.29), C1-4926: I.A + (H.4.9) + (B.1.29), C1-4927: I.A +
(H.4.10) + (B.1.29), C1-4928: I.A + (I.2.2) + (B.1.29), C1-4929: I.A + (I.2.5) + (B.1.29), C1-4930:
I.A + (J.1.2) + (B.1.29), C1-4931: I.A + (J.1.5) + (B.1.29), C1-4932: I.A + (J.1.8) + (B.1.29), C1-
4933: I.A + (J.1.11) + (B.1.29), C1-4934: I.A + (J.1.12) + (B.1.29), C1-4935: I.A + (K.1.3) +
(B.1.29), C1-4936: I.A + (K.1.29) + (B.1.29), C1-4937: I.A + (K.1.30) + (B.1.29), C1-4938: I.A +
(K.1.31) + (B.1.29), C1-4939: I.A + (K.1.32) + (B.1.29), C1-4940: I.A + (K.1.41) + (B.1.29), C1-
4941: I.A + (K.1.42) + (B.1.29), C1-4942: I.A + (K.1.44) + (B.1.29), C1-4943: I.A + (K.1.45) +
(B.1.29), C1-4944: I.A + (K.1.47) + (B.1.29), C1-4945: I.A + (K.1.49) + (B.1.29), C1-4946: I.A +
(K.1.53) + (B.1.29), C1-4947: I.A + (K.1.54) + (B.1.29), C1-4948: I.A + (A.1.4) + (B.1.38), C1-
4949: I.A + (A.1.12) + (B.1.38), C1-4950: I.A + (A.1.21) + (B.1.38), C1-4951: I.A + (A.1,21a) +
(B.1.38), C1-4952: I.A + (A.1.24) + (B.1.38), C1-4953: I.A + (A.1.26) + (B.1.38), C1-4954: I.A +
(A.1.27) + (B.1.38), C1-4955: I.A + (A.1.30) + (B.1.38), C1-4956: I.A + (A.1.31) + (B.1.38), C1-
4957: I.A + (A.1.32) + (B.1.38), C1-4958: I.A + (A.1.35) + (B.1.38), C1-4959: I.A + (A.2.1) +
(B.1.38), C1-4960: I.A + (A.2.3) + (B.1.38), C1-4961: I.A + (A.2.5) + (B.1.38), C1-4962: I.A +
(A.3.11) + (B.1.38), C1-4963: I.A + (A.3.12) + (B.1.38), C1-4964: I.A + (A.3.15) + (B.1.38), C1-
4965: I.A + (A.3.16) + (B.1.38), C1-4966: I.A + (A.3.18) + (B.1.38), C1-4967: I.A + (A.3.19) +
(B.1.38), C1-4968: I.A + (A.3.20) + (B.1.38), C1-4969: I.A + (A.3.21) + (B.1.38), C1-4970: I.A +
(A.3.22) + (B.1.38), C1-4971: I.A + (A.3.23) + (B.1.38), C1-4972: I.A + (A.3.25) + (B.1.38), C1-
4973: I.A + (A.3.26) + (B.1.38), C1-4974: I.A + (A.3.27) + (B.1.38), C1-4975: I.A + (A.4.5) +
(B.1.38), C1-4976: I.A + (A.4.11) + (B.1.38), C1-4977: I.A + (B.1.10) + (B.1.38), C1-4978: I.A +
(B.1.11) + (B.1.38), C1-4979: I.A + (B.1.12) + (B.1.38), C1-4980: I.A + (B.1.13) + (B.1.38), C1-
4981: I.A + (B.1.18) + (B.1.38), C1-4982: I.A + (B.1.21) + (B.1.38), C1-4983: I.A + (B.1.26) +
(B.1.38), C1-4984: I.A + (B.1.33) + (B.1.38), C1-4985: I.A + (B.1.34) + (B.1.38), C1-4986: I.A +
(B.1.37) + (B.1.38), C1-4987: I.A + (B.1.43) + (B.1.38), C1-4988: I.A + (B.1.46) + (B.1.38), C1-
4989: I.A + (B.2.4) + (B.1.38), C1-4990: I.A + (B.2.5) + (B.1.38), C1-4991: I.A + (B.2.6) +
(B.1.38), C1-4992: I.A + (B.2.8) + (B.1.38), C1-4993: I.A + (C.1.1) + (B.1.38), C1-4994: I.A +
(C.1.2) + (B.1.38), C1-4995: I.A + (C.1.4) + (B.1.38), C1-4996: I.A + (C.1.5) + (B.1.38), C1-
4997: I.A + (C.2.6) + (B.1.38), C1-4998: I.A + (C.2.7) + (B.1.38), C1-4999: I.A + (C.2.8) +
(B.1.38), C1-5000: I.A + (D.1.1) + (B.1.38), C1-5001: I.A + (D.1.2) + (B.1.38), C1-5002: I.A +
(D.1.5) + (B.1.38), C1-5003: I.A + (D.2.4) + (B.1.38), C1-5004: I.A + (D.2.6) + (B.1.38), C1-
5005: I.A + (D.2.7) + (B.1.38), C1-5006: I.A + (E.1.1) + (B.1.38), C1-5007: I.A + (E.1.3) +
(B.1.38), C1-5008: I.A + (E.2.2) + (B.1.38), C1-5009: I.A + (E.2.3) + (B.1.38), C1-5010: I.A +
(F.1.2) + (B.1.38), C1-5011: I.A + (F.1.4) + (B.1.38), C1-5012: I.A + (F.1.5) + (B.1.38), C1-5013:
I.A + (G.3.1) + (B.1.38), C1-5014: I.A + (G.3.3) + (B.1.38), C1-5015: I.A + (G.3.6) + (B.1.38),
C1-5016: I.A + (G.5.1) + (B.1.38), C1-5017: I.A + (G.5.2) + (B.1.38), C1-5018: I.A + (G.5.3) +
(B.1.38), C1-5019: I.A + (G.5.4) + (B.1.38), C1-5020: I.A + (G.5.5) + (B.1.38), C1-5021: I.A +
(G.5.6) + (B.1.38), C1-5022: I.A + (G.5.7) + (B.1.38), C1-5023: I.A + (G.5.8) + (B.1.38), C1-
5024: I.A + (G.5.9) + (B.1.38), C1-5025: I.A + (G.5.10) + (B.1.38), C1-5026: I.A + (G.5.11) +
(B.1.38), C1-5027: I.A + (H.1.5) + (B.1.38), C1-5028: I.A + (H.1.7) + (B.1.38), C1-5029: I.A +
(H.2.2) + (B.1.38), C1-5030: I.A + (H.2.3) + (B.1.38), C1-5031: I.A + (H.2.5) + (B.1.38), C1-
5032: I.A + (H.2.7) + (B.1.38), C1-5033: I.A + (H.2.8) + (B.1.38), C1-5034: I.A + (H.3.2) +
(B.1.38), C1-5035: I.A + (H.3.4) + (B.1.38), C1-5036: I.A + (H.3.5) + (B.1.38), C1-5037: I.A +
(H.4.9) + (B.1.38), C1-5038: I.A + (H.4.10) + (B.1.38), C1-5039: I.A + (I.2.2) + (B.1.38), C1-
5040: I.A + (I.2.5) + (B.1.38), C1-5041: I.A + (J.1.2) + (B.1.38), C1-5042: I.A + (J.1.5) + (B.1.38),
C1-5043: I.A + (J.1.8) + (B.1.38), C1-5044: I.A + (J.1.11) + (B.1.38), C1-5045: I.A + (J.1.12) +
(B.1.38), C1-5046: I.A + (K.1.3) + (B.1.38), C1-5047: I.A + (K.1.29) + (B.1.38), C1-5048: I.A +
(K.1.30) + (B.1.38), C1-5049: I.A + (K.1.31) + (B.1.38), C1-5050: I.A + (K.1.32) + (B.1.38), C1-
5051: I.A + (K.1.41) + (B.1.38), C1-5052: I.A + (K.1.42) + (B.1.38), C1-5053: I.A + (K.1.44) +
(B.1.38), C1-5054: I.A + (K.1.45) + (B.1.38), C1-5055: I.A + (K.1.47) + (B.1.38), C1-5056: I.A +
(K.1.49) + (B.1.38), C1-5057: I.A + (K.1.53) + (B.1.38), C1-5058: I.A + (K.1.54) + (B.1.38), C1-
5059: I.A + (A.1.4) + (B.2.4), C1-5060: I.A + (A.1.12) + (B.2.4), C1-5061: I.A + (A.1.21) +
(B.2.4), C1-5062: I.A + (A.1,21a) + (B.2.4), C1-5063: I.A + (A.1.24) + (B.2.4), C1-5064: I.A +
(A.1.26) + (B.2.4), C1-5065: I.A + (A.1.27) + (B.2.4), C1-5066: I.A + (A.1.30) + (B.2.4), C1-5067:
I.A + (A.1.31) + (B.2.4), C1-5068: I.A + (A.1.32) + (B.2.4), C1-5069: I.A + (A.1.35) + (B.2.4), C1-
5070: I.A + (A.2.1) + (B.2.4), C1-5071: I.A + (A.2.3) + (B.2.4), C1-5072: I.A + (A.2.5) + (B.2.4),
C1-5073: I.A + (A.3.11) + (B.2.4), C1-5074: I.A + (A.3.12) + (B.2.4), C1-5075: I.A + (A.3.15) +
(B.2.4), C1-5076: I.A + (A.3.16) + (B.2.4), C1-5077: I.A + (A.3.18) + (B.2.4), C1-5078: I.A +
(A.3.19) + (B.2.4), C1-5079: I.A + (A.3.20) + (B.2.4), C1-5080: I.A + (A.3.21) + (B.2.4), C1-5081:
I.A + (A.3.22) + (B.2.4), C1-5082: I.A + (A.3.23) + (B.2.4), C1-5083: I.A + (A.3.25) + (B.2.4), C1-
5084: I.A + (A.3.26) + (B.2.4), C1-5085: I.A + (A.3.27) + (B.2.4), C1-5086: I.A + (A.4.5) +
(B.2.4), C1-5087: I.A + (A.4.11) + (B.2.4), C1-5088: I.A + (B.1.10) + (B.2.4), C1-5089: I.A +
(B.1.11) + (B.2.4), C1-5090: I.A + (B.1.12) + (B.2.4), C1-5091: I.A + (B.1.13) + (B.2.4), C1-5092:
I.A + (B.1.18) + (B.2.4), C1-5093: I.A + (B.1.21) + (B.2.4), C1-5094: I.A + (B.1.26) + (B.2.4), C1-
5095: I.A + (B.1.33) + (B.2.4), C1-5096: I.A + (B.1.34) + (B.2.4), C1-5097: I.A + (B.1.37) +
(B.2.4), C1-5098: I.A + (B.1.43) + (B.2.4), C1-5099: I.A + (B.1.46) + (B.2.4), C1-5100: I.A +
(B.2.5) + (B.2.4), C1-5101: I.A + (B.2.6) + (B.2.4), C1-5102: I.A + (B.2.8) + (B.2.4), C1-5103: I.A +
(C.1.1) + (B.2.4), C1-5104: I.A + (C.1.2) + (B.2.4), C1-5105: I.A + (C.1.4) + (B.2.4), C1-5106:
I.A + (C.1.5) + (B.2.4), C1-5107: I.A + (C.2.6) + (B.2.4), C1-5108: I.A + (C.2.7) + (B.2.4), C1-
5109: I.A + (C.2.8) + (B.2.4), C1-5110: I.A + (D.1.1) + (B.2.4), C1-5111: I.A + (D.1.2) + (B.2.4),
C1-5112: I.A + (D.1.5) + (B.2.4), C1-5113: I.A + (D.2.4) + (B.2.4), C1-5114: I.A + (D.2.6) +
(B.2.4), C1-5115: I.A + (D.2.7) + (B.2.4), C1-5116: I.A + (E.1.1) + (B.2.4), C1-5117: I.A + (E.1.3) +
(B.2.4), C1-5118: I.A + (E.2.2) + (B.2.4), C1-5119: I.A + (E.2.3) + (B.2.4), C1-5120: I.A +
(F.1.2) + (B.2.4), C1-5121: I.A + (F.1.4) + (B.2.4), C1-5122: I.A + (F.1.5) + (B.2.4), C1-5123: I.A +
(G.3.1) + (B.2.4), C1-5124: I.A + (G.3.3) + (B.2.4), C1-5125: I.A + (G.3.6) + (B.2.4), C1-5126:
I.A + (G.5.1) + (B.2.4), C1-5127: I.A + (G.5.2) + (B.2.4), C1-5128: I.A + (G.5.3) + (B.2.4), C1-
5129: I.A + (G.5.4) + (B.2.4), C1-5130: I.A + (G.5.5) + (B.2.4), C1-5131: I.A + (G.5.6) + (B.2.4),
C1-5132: I.A + (G.5.7) + (B.2.4), C1-5133: I.A + (G.5.8) + (B.2.4), C1-5134: I.A + (G.5.9) +
(B.2.4), C1-5135: I.A + (G.5.10) + (B.2.4), C1-5136: I.A + (G.5.11) + (B.2.4), C1-5137: I.A +
(H.1.5) + (B.2.4), C1-5138: I.A + (H.1.7) + (B.2.4), C1-5139: I.A + (H.2.2) + (B.2.4), C1-5140: I.A +
(H.2.3) + (B.2.4), C1-5141: I.A + (H.2.5) + (B.2.4), C1-5142: I.A + (H.2.7) + (B.2.4), C1-5143:
I.A + (H.2.8) + (B.2.4), C1-5144: I.A + (H.3.2) + (B.2.4), C1-5145: I.A + (H.3.4) + (B.2.4), C1-
5146: I.A + (H.3.5) + (B.2.4), C1-5147: I.A + (H.4.9) + (B.2.4), C1-5148: I.A + (H.4.10) + (B.2.4),
C1-5149: I.A + (I.2.2) + (B.2.4), C1-5150: I.A + (I.2.5) + (B.2.4), C1-5151: I.A + (J.1.2) + (B.2.4),
C1-5152: I.A + (J.1.5) + (B.2.4), C1-5153: I.A + (J.1.8) + (B.2.4), C1-5154: I.A + (J.1.11) +
(B.2.4), C1-5155: I.A + (J.1.12) + (B.2.4), C1-5156: I.A + (K.1.3) + (B.2.4), C1-5157: I.A +
(K.1.29) + (B.2.4), C1-5158: I.A + (K.1.30) + (B.2.4), C1-5159: I.A + (K.1.31) + (B.2.4), C1-5160:
I.A + (K.1.32) + (B.2.4), C1-5161: I.A + (K.1.41) + (B.2.4), C1-5162: I.A + (K.1.42) + (B.2.4), C1-
5163: I.A + (K.1.44) + (B.2.4), C1-5164: I.A + (K.1.45) + (B.2.4), C1-5165: I.A + (K.1.47) +
(B.2.4), C1-5166: I.A + (K.1.49) + (B.2.4), C1-5167: I.A + (K.1.53) + (B.2.4), C1-5168: I.A +
(K.1.54) + (B.2.4), C1-5169: I.A + (A.1.4) + (B.2.6), C1-5170: I.A + (A.1.12) + (B.2.6), C1-5171:
I.A + (A.1.21) + (B.2.6), C1-5172: I.A + (A.1,21a) + (B.2.6), C1-5173: I.A + (A.1.24) + (B.2.6),
C1-5174: I.A + (A.1.26) + (B.2.6), C1-5175: I.A + (A.1.27) + (B.2.6), C1-5176: I.A + (A.1.30) +
(B.2.6), C1-5177: I.A + (A.1.31) + (B.2.6), C1-5178: I.A + (A.1.32) + (B.2.6), C1-5179: I.A +
(A.1.35) + (B.2.6), C1-5180: I.A + (A.2.1) + (B.2.6), C1-5181: I.A + (A.2.3) + (B.2.6), C1-5182:
I.A + (A.2.5) + (B.2.6), C1-5183: I.A + (A.3.11) + (B.2.6), C1-5184: I.A + (A.3.12) + (B.2.6), C1-
5185: I.A + (A.3.15) + (B.2.6), C1-5186: I.A + (A.3.16) + (B.2.6), C1-5187: I.A + (A.3.18) +
(B.2.6), C1-5188: I.A + (A.3.19) + (B.2.6), C1-5189: I.A + (A.3.20) + (B.2.6), C1-5190: I.A +
(A.3.21) + (B.2.6), C1-5191: I.A + (A.3.22) + (B.2.6), C1-5192: I.A + (A.3.23) + (B.2.6), C1-5193:
I.A + (A.3.25) + (B.2.6), C1-5194: I.A + (A.3.26) + (B.2.6), C1-5195: I.A + (A.3.27) + (B.2.6), C1-
5196: I.A + (A.4.5) + (B.2.6), C1-5197: I.A + (A.4.11) + (B.2.6), C1-5198: I.A + (B.1.10) +
(B.2.6), C1-5199: I.A + (B.1.11) + (B.2.6), C1-5200: I.A + (B.1.12) + (B.2.6), C1-5201: I.A +
(B.1.13) + (B.2.6), C1-5202: I.A + (B.1.18) + (B.2.6), C1-5203: I.A + (B.1.21) + (B.2.6), C1-5204:
I.A + (B.1.26) + (B.2.6), C1-5205: I.A + (B.1.33) + (B.2.6), C1-5206: I.A + (B.1.34) + (B.2.6), C1-
5207: I.A + (B.1.37) + (B.2.6), C1-5208: I.A + (B.1.43) + (B.2.6), C1-5209: I.A + (B.1.46) +
(B.2.6), C1-5210: I.A + (B.2.5) + (B.2.6), C1-5211: I.A + (B.2.8) + (B.2.6), C1-5212: I.A + (C.1.1) +
(B.2.6), C1-5213: I.A + (C.1.2) + (B.2.6), C1-5214: I.A + (C.1.4) + (B.2.6), C1-5215: I.A +
(C.1.5) + (B.2.6), C1-5216: I.A + (C.2.6) + (B.2.6), C1-5217: I.A + (C.2.7) + (B.2.6), C1-5218: I.A +
(C.2.8) + (B.2.6), C1-5219: I.A + (D.1.1) + (B.2.6), C1-5220: I.A + (D.1.2) + (B.2.6), C1-5221:
I.A + (D.1.5) + (B.2.6), C1-5222: I.A + (D.2.4) + (B.2.6), C1-5223: I.A + (D.2.6) + (B.2.6), C1-
5224: I.A + (D.2.7) + (B.2.6), C1-5225: I.A + (E.1.1) + (B.2.6), C1-5226: I.A + (E.1.3) + (B.2.6),
C1-5227: I.A + (E.2.2) + (B.2.6), C1-5228: I.A + (E.2.3) + (B.2.6), C1-5229: I.A + (F.1.2) +
(B.2.6), C1-5230: I.A + (F.1.4) + (B.2.6), C1-5231: I.A + (F.1.5) + (B.2.6), C1-5232: I.A + (G.3.1) +
(B.2.6), C1-5233: I.A + (G.3.3) + (B.2.6), C1-5234: I.A + (G.3.6) + (B.2.6), C1-5235: I.A +
(G.5.1) + (B.2.6), C1-5236: I.A + (G.5.2) + (B.2.6), C1-5237: I.A + (G.5.3) + (B.2.6), C1-5238:
I.A + (G.5.4) + (B.2.6), C1-5239: I.A + (G.5.5) + (B.2.6), C1-5240: I.A + (G.5.6) + (B.2.6), C1-
5241: I.A + (G.5.7) + (B.2.6), C1-5242: I.A + (G.5.8) + (B.2.6), C1-5243: I.A + (G.5.9) + (B.2.6),
C1-5244: I.A + (G.5.10) + (B.2.6), C1-5245: I.A + (G.5.11) + (B.2.6), C1-5246: I.A + (H.1.5) +
(B.2.6), C1-5247: I.A + (H.1.7) + (B.2.6), C1-5248: I.A + (H.2.2) + (B.2.6), C1-5249: I.A + (H.2.3) +
(B.2.6), C1-5250: I.A + (H.2.5) + (B.2.6), C1-5251: I.A + (H.2.7) + (B.2.6), C1-5252: I.A +
(H.2.8) + (B.2.6), C1-5253: I.A + (H.3.2) + (B.2.6), C1-5254: I.A + (H.3.4) + (B.2.6), C1-5255: I.A +
(H.3.5) + (B.2.6), C1-5256: I.A + (H.4.9) + (B.2.6), C1-5257: I.A + (H.4.10) + (B.2.6), C1-5258:
I.A + (I.2.2) + (B.2.6), C1-5259: I.A + (I.2.5) + (B.2.6), C1-5260: I.A + (J.1.2) + (B.2.6), C1-5261:
I.A + (J.1.5) + (B.2.6), C1-5262: I.A + (J.1.8) + (B.2.6), C1-5263: I.A + (J.1.11) + (B.2.6), C1-
5264: I.A + (J.1.12) + (B.2.6), C1-5265: I.A + (K.1.3) + (B.2.6), C1-5266: I.A + (K.1.29) + (B.2.6),
C1-5267: I.A + (K.1.30) + (B.2.6), C1-5268: I.A + (K.1.31) + (B.2.6), C1-5269: I.A + (K.1.32) +
(B.2.6), C1-5270: I.A + (K.1.41) + (B.2.6), C1-5271: I.A + (K.1.42) + (B.2.6), C1-5272: I.A +
(K.1.44) + (B.2.6), C1-5273: I.A + (K.1.45) + (B.2.6), C1-5274: I.A + (K.1.47) + (B.2.6), C1-5275:
I.A + (K.1.49) + (B.2.6), C1-5276: I.A + (K.1.53) + (B.2.6), C1-5277: I.A + (K.1.54) + (B.2.6), C1-
5278: I.A + (A.1.4) + (D.2.6), C1-5279: I.A + (A.1.12) + (D.2.6), C1-5280: I.A + (A.1.21) +
(D.2.6), C1-5281: I.A + (A.1,21a) + (D.2.6), C1-5282: I.A + (A.1.24) + (D.2.6), C1-5283: I.A +
(A.1.26) + (D.2.6), C1-5284: I.A + (A.1.27) + (D.2.6), C1-5285: I.A + (A.1.30) + (D.2.6), C1-
5286: I.A + (A.1.31) + (D.2.6), C1-5287: I.A + (A.1.32) + (D.2.6), C1-5288: I.A + (A.1.35) +
(D.2.6), C1-5289: I.A + (A.2.1) + (D.2.6), C1-5290: I.A + (A.2.3) + (D.2.6), C1-5291: I.A + (A.2.5) +
(D.2.6), C1-5292: I.A + (A.3.11) + (D.2.6), C1-5293: I.A + (A.3.12) + (D.2.6), C1-5294: I.A +
(A.3.15) + (D.2.6), C1-5295: I.A + (A.3.16) + (D.2.6), C1-5296: I.A + (A.3.18) + (D.2.6), C1-
5297: I.A + (A.3.19) + (D.2.6), C1-5298: I.A + (A.3.20) + (D.2.6), C1-5299: I.A + (A.3.21) +
(D.2.6), C1-5300: I.A + (A.3.22) + (D.2.6), C1-5301: I.A + (A.3.23) + (D.2.6), C1-5302: I.A +
(A.3.25) + (D.2.6), C1-5303: I.A + (A.3.26) + (D.2.6), C1-5304: I.A + (A.3.27) + (D.2.6), C1-
5305: I.A + (A.4.5) + (D.2.6), C1-5306: I.A + (A.4.11) + (D.2.6), C1-5307: I.A + (B.1.10) +
(D.2.6), C1-5308: I.A + (B.1.11) + (D.2.6), C1-5309: I.A + (B.1.12) + (D.2.6), C1-5310: I.A +
(B.1.13) + (D.2.6), C1-5311: I.A + (B.1.18) + (D.2.6), C1-5312: I.A + (B.1.21) + (D.2.6), C1-
5313: I.A + (B.1.26) + (D.2.6), C1-5314: I.A + (B.1.33) + (D.2.6), C1-5315: I.A + (B.1.34) +
(D.2.6), C1-5316: I.A + (B.1.37) + (D.2.6), C1-5317: I.A + (B.1.43) + (D.2.6), C1-5318: I.A +
(B.1.46) + (D.2.6), C1-5319: I.A + (B.2.5) + (D.2.6), C1-5320: I.A + (B.2.8) + (D.2.6), C1-5321:
I.A + (C.1.1) + (D.2.6), C1-5322: I.A + (C.1.2) + (D.2.6), C1-5323: I.A + (C.1.4) + (D.2.6), C1-
5324: I.A + (C.1.5) + (D.2.6), C1-5325: I.A + (C.2.6) + (D.2.6), C1-5326: I.A + (C.2.7) + (D.2.6),
C1-5327: I.A + (C.2.8) + (D.2.6), C1-5328: I.A + (D.1.1) + (D.2.6), C1-5329: I.A + (D.1.2) +
(D.2.6), C1-5330: I.A + (D.1.5) + (D.2.6), C1-5331: I.A + (D.2.4) + (D.2.6), C1-5332: I.A +
(D.2.7) + (D.2.6), C1-5333: I.A + (E.1.1) + (D.2.6), C1-5334: I.A + (E.1.3) + (D.2.6), C1-5335: I.A +
(E.2.2) + (D.2.6), C1-5336: I.A + (E.2.3) + (D.2.6), C1-5337: I.A + (F.1.2) + (D.2.6), C1-5338:
I.A + (F.1.4) + (D.2.6), C1-5339: I.A + (F.1.5) + (D.2.6), C1-5340: I.A + (G.3.1) + (D.2.6), C1-
5341: I.A + (G.3.3) + (D.2.6), C1-5342: I.A + (G.3.6) + (D.2.6), C1-5343: I.A + (G.5.1) + (D.2.6),
C1-5344: I.A + (G.5.2) + (D.2.6), C1-5345: I.A + (G.5.3) + (D.2.6), C1-5346: I.A + (G.5.4) +
(D.2.6), C1-5347: I.A + (G.5.5) + (D.2.6), C1-5348: I.A + (G.5.6) + (D.2.6), C1-5349: I.A +
(G.5.7) + (D.2.6), C1-5350: I.A + (G.5.8) + (D.2.6), C1-5351: I.A + (G.5.9) + (D.2.6), C1-5352:
I.A + (G.5.10) + (D.2.6), C1-5353: I.A + (G.5.11) + (D.2.6), C1-5354: I.A + (H.1.5) + (D.2.6), C1-
5355: I.A + (H.1.7) + (D.2.6), C1-5356: I.A + (H.2.2) + (D.2.6), C1-5357: I.A + (H.2.3) + (D.2.6),
C1-5358: I.A + (H.2.5) + (D.2.6), C1-5359: I.A + (H.2.7) + (D.2.6), C1-5360: I.A + (H.2.8) +
(D.2.6), C1-5361: I.A + (H.3.2) + (D.2.6), C1-5362: I.A + (H.3.4) + (D.2.6), C1-5363: I.A +
(H.3.5) + (D.2.6), C1-5364: I.A + (H.4.9) + (D.2.6), C1-5365: I.A + (H.4.10) + (D.2.6), C1-5366:
I.A + (I.2.2) + (D.2.6), C1-5367: I.A + (I.2.5) + (D.2.6), C1-5368: I.A + (J.1.2) + (D.2.6), C1-5369:
I.A + (J.1.5) + (D.2.6), C1-5370: I.A + (J.1.8) + (D.2.6), C1-5371: I.A + (J.1.11) + (D.2.6), C1-
5372: I.A + (J.1.12) + (D.2.6), C1-5373: I.A + (K.1.3) + (D.2.6), C1-5374: I.A + (K.1.29) +
(D.2.6), C1-5375: I.A + (K.1.30) + (D.2.6), C1-5376: I.A + (K.1.31) + (D.2.6), C1-5377: I.A +
(K.1.32) + (D.2.6), C1-5378: I.A + (K.1.41) + (D.2.6), C1-5379: I.A + (K.1.42) + (D.2.6), C1-
5380: I.A + (K.1.44) + (D.2.6), C1-5381: I.A + (K.1.45) + (D.2.6), C1-5382: I.A + (K.1.47) +
(D.2.6), C1-5383: I.A + (K.1.49) + (D.2.6), C1-5384: I.A + (K.1.53) + (D.2.6), C1-5385: I.A +
(K.1.54) + (D.2.6), C1-5386: I.A + (A.1.4) + (D.2.7), C1-5387: I.A + (A.1.12) + (D.2.7), C1-5388:
I.A + (A.1.21) + (D.2.7), C1-5389: I.A + (A.1,21a) + (D.2.7), C1-5390: I.A + (A.1.24) + (D.2.7),
C1-5391: I.A + (A.1.26) + (D.2.7), C1-5392: I.A + (A.1.27) + (D.2.7), C1-5393: I.A + (A.1.30) +
(D.2.7), C1-5394: I.A + (A.1.31) + (D.2.7), C1-5395: I.A + (A.1.32) + (D.2.7), C1-5396: I.A +
(A.1.35) + (D.2.7), C1-5397: I.A + (A.2.1) + (D.2.7), C1-5398: I.A + (A.2.3) + (D.2.7), C1-5399:
I.A + (A.2.5) + (D.2.7), C1-5400: I.A + (A.3.11) + (D.2.7), C1-5401: I.A + (A.3.12) + (D.2.7), C1-
5402: I.A + (A.3.15) + (D.2.7), C1-5403: I.A + (A.3.16) + (D.2.7), C1-5404: I.A + (A.3.18) +
(D.2.7), C1-5405: I.A + (A.3.19) + (D.2.7), C1-5406: I.A + (A.3.20) + (D.2.7), C1-5407: I.A +
(A.3.21) + (D.2.7), C1-5408: I.A + (A.3.22) + (D.2.7), C1-5409: I.A + (A.3.23) + (D.2.7), C1-
5410: I.A + (A.3.25) + (D.2.7), C1-5411: I.A + (A.3.26) + (D.2.7), C1-5412: I.A + (A.3.27) +
(D.2.7), C1-5413: I.A + (A.4.5) + (D.2.7), C1-5414: I.A + (A.4.11) + (D.2.7), C1-5415: I.A +
(B.1.10) + (D.2.7), C1-5416: I.A + (B.1.11) + (D.2.7), C1-5417: I.A + (B.1.12) + (D.2.7), C1-
5418: I.A + (B.1.13) + (D.2.7), C1-5419: I.A + (B.1.18) + (D.2.7), C1-5420: I.A + (B.1.21) +
(D.2.7), C1-5421: I.A + (B.1.26) + (D.2.7), C1-5422: I.A + (B.1.33) + (D.2.7), C1-5423: I.A +
(B.1.34) + (D.2.7), C1-5424: I.A + (B.1.37) + (D.2.7), C1-5425: I.A + (B.1.43) + (D.2.7), C1-
5426: I.A + (B.1.46) + (D.2.7), C1-5427: I.A + (B.2.5) + (D.2.7), C1-5428: I.A + (B.2.8) + (D.2.7),
C1-5429: I.A + (C.1.1) + (D.2.7), C1-5430: I.A + (C.1.2) + (D.2.7), C1-5431: I.A + (C.1.4) +
(D.2.7), C1-5432: I.A + (C.1.5) + (D.2.7), C1-5433: I.A + (C.2.6) + (D.2.7), C1-5434: I.A +
(C.2.7) + (D.2.7), C1-5435: I.A + (C.2.8) + (D.2.7), C1-5436: I.A + (D.1.1) + (D.2.7), C1-5437:
I.A + (D.1.2) + (D.2.7), C1-5438: I.A + (D.1.5) + (D.2.7), C1-5439: I.A + (D.2.4) + (D.2.7), C1-
5440: I.A + (E.1.1) + (D.2.7), C1-5441: I.A + (E.1.3) + (D.2.7), C1-5442: I.A + (E.2.2) + (D.2.7),
C1-5443: I.A + (E.2.3) + (D.2.7), C1-5444: I.A + (F.1.2) + (D.2.7), C1-5445: I.A + (F.1.4) +
(D.2.7), C1-5446: I.A + (F.1.5) + (D.2.7), C1-5447: I.A + (G.3.1) + (D.2.7), C1-5448: I.A +
(G.3.3) + (D.2.7), C1-5449: I.A + (G.3.6) + (D.2.7), C1-5450: I.A + (G.5.1) + (D.2.7), C1-5451:
I.A + (G.5.2) + (D.2.7), C1-5452: I.A + (G.5.3) + (D.2.7), C1-5453: I.A + (G.5.4) + (D.2.7), C1-
5454: I.A + (G.5.5) + (D.2.7), C1-5455: I.A + (G.5.6) + (D.2.7), C1-5456: I.A + (G.5.7) + (D.2.7),
C1-5457: I.A + (G.5.8) + (D.2.7), C1-5458: I.A + (G.5.9) + (D.2.7), C1-5459: I.A + (G.5.10) +
(D.2.7), C1-5460: I.A + (G.5.11) + (D.2.7), C1-5461: I.A + (H.1.5) + (D.2.7), C1-5462: I.A +
(H.1.7) + (D.2.7), C1-5463: I.A + (H.2.2) + (D.2.7), C1-5464: I.A + (H.2.3) + (D.2.7), C1-5465:
I.A + (H.2.5) + (D.2.7), C1-5466: I.A + (H.2.7) + (D.2.7), C1-5467: I.A + (H.2.8) + (D.2.7), C1-
5468: I.A + (H.3.2) + (D.2.7), C1-5469: I.A + (H.3.4) + (D.2.7), C1-5470: I.A + (H.3.5) + (D.2.7),
C1-5471: I.A + (H.4.9) + (D.2.7), C1-5472: I.A + (H.4.10) + (D.2.7), C1-5473: I.A + (I.2.2) +
(D.2.7), C1-5474: I.A + (I.2.5) + (D.2.7), C1-5475: I.A + (J.1.2) + (D.2.7), C1-5476: I.A + (J.1.5) +
(D.2.7), C1-5477: I.A + (J.1.8) + (D.2.7), C1-5478: I.A + (J.1.11) + (D.2.7), C1-5479: I.A +
(J.1.12) + (D.2.7), C1-5480: I.A + (K.1.3) + (D.2.7), C1-5481: I.A + (K.1.29) + (D.2.7), C1-5482:
I.A + (K.1.30) + (D.2.7), C1-5483: I.A + (K.1.31) + (D.2.7), C1-5484: I.A + (K.1.32) + (D.2.7), C1-
5485: I.A + (K.1.41) + (D.2.7), C1-5486: I.A + (K.1.42) + (D.2.7), C1-5487: I.A + (K.1.44) +
(D.2.7), C1-5488: I.A + (K.1.45) + (D.2.7), C1-5489: I.A + (K.1.47) + (D.2.7), C1-5490: I.A +
(K.1.49) + (D.2.7), C1-5491: I.A + (K.1.53) + (D.2.7), C1-5492: I.A + (K.1.54) + (D.2.7), C1-
5493: I.A + (A.1.4) + (E.1.1), C1-5494: I.A + (A.1.12) + (E.1.1), C1-5495: I.A + (A.1.21) +
(E.1.1), C1-5496: I.A + (A.1,21a) + (E.1.1), C1-5497: I.A + (A.1.24) + (E.1.1), C1-5498: I.A +
(A.1.26) + (E.1.1), C1-5499: I.A + (A.1.27) + (E.1.1), C1-5500: I.A + (A.1.30) + (E.1.1), C1-5501:
I.A + (A.1.31) + (E.1.1), C1-5502: I.A + (A.1.32) + (E.1.1), C1-5503: I.A + (A.1.35) + (E.1.1), C1-
5504: I.A + (A.2.1) + (E.1.1), C1-5505: I.A + (A.2.3) + (E.1.1), C1-5506: I.A + (A.2.5) + (E.1.1),
C1-5507: I.A + (A.3.11) + (E.1.1), C1-5508: I.A + (A.3.12) + (E.1.1), C1-5509: I.A + (A.3.15) +
(E.1.1), C1-5510: I.A + (A.3.16) + (E.1.1), C1-5511: I.A + (A.3.18) + (E.1.1), C1-5512: I.A +
(A.3.19) + (E.1.1), C1-5513: I.A + (A.3.20) + (E.1.1), C1-5514: I.A + (A.3.21) + (E.1.1), C1-5515:
I.A + (A.3.22) + (E.1.1), C1-5516: I.A + (A.3.23) + (E.1.1), C1-5517: I.A + (A.3.25) + (E.1.1), C1-
5518: I.A + (A.3.26) + (E.1.1), C1-5519: I.A + (A.3.27) + (E.1.1), C1-5520: I.A + (A.4.5) +
(E.1.1), C1-5521: I.A + (A.4.11) + (E.1.1), C1-5522: I.A + (B.1.10) + (E.1.1), C1-5523: I.A +
(B.1.11) + (E.1.1), C1-5524: I.A + (B.1.12) + (E.1.1), C1-5525: I.A + (B.1.13) + (E.1.1), C1-5526:
I.A + (B.1.18) + (E.1.1), C1-5527: I.A + (B.1.21) + (E.1.1), C1-5528: I.A + (B.1.26) + (E.1.1), C1-
5529: I.A + (B.1.33) + (E.1.1), C1-5530: I.A + (B.1.34) + (E.1.1), C1-5531: I.A + (B.1.37) +
(E.1.1), C1-5532: I.A + (B.1.43) + (E.1.1), C1-5533: I.A + (B.1.46) + (E.1.1), C1-5534: I.A +
(B.2.5) + (E.1.1), C1-5535: I.A + (B.2.8) + (E.1.1), C1-5536: I.A + (C.1.1) + (E.1.1), C1-5537: I.A +
(C.1.2) + (E.1.1), C1-5538: I.A + (C.1.4) + (E.1.1), C1-5539: I.A + (C.1.5) + (E.1.1), C1-5540:
I.A + (C.2.6) + (E.1.1), C1-5541: I.A + (C.2.7) + (E.1.1), C1-5542: I.A + (C.2.8) + (E.1.1), C1-
5543: I.A + (D.1.1) + (E.1.1), C1-5544: I.A + (D.1.2) + (E.1.1), C1-5545: I.A + (D.1.5) + (E.1.1),
C1-5546: I.A + (D.2.4) + (E.1.1), C1-5547: I.A + (E.1.3) + (E.1.1), C1-5548: I.A + (E.2.2) +
(E.1.1), C1-5549: I.A + (E.2.3) + (E.1.1), C1-5550: I.A + (F.1.2) + (E.1.1), C1-5551: I.A + (F.1.4) +
(E.1.1), C1-5552: I.A + (F.1.5) + (E.1.1), C1-5553: I.A + (G.3.1) + (E.1.1), C1-5554: I.A +
(G.3.3) + (E.1.1), C1-5555: I.A + (G.3.6) + (E.1.1), C1-5556: I.A + (G.5.1) + (E.1.1), C1-5557:
I.A + (G.5.2) + (E.1.1), C1-5558: I.A + (G.5.3) + (E.1.1), C1-5559: I.A + (G.5.4) + (E.1.1), C1-
5560: I.A + (G.5.5) + (E.1.1), C1-5561: I.A + (G.5.6) + (E.1.1), C1-5562: I.A + (G.5.7) + (E.1.1),
C1-5563: I.A + (G.5.8) + (E.1.1), C1-5564: I.A + (G.5.9) + (E.1.1), C1-5565: I.A + (G.5.10) +
(E.1.1), C1-5566: I.A + (G.5.11) + (E.1.1), C1-5567: I.A + (H.1.5) + (E.1.1), C1-5568: I.A +
(H.1.7) + (E.1.1), C1-5569: I.A + (H.2.2) + (E.1.1), C1-5570: I.A + (H.2.3) + (E.1.1), C1-5571: I.A +
(H.2.5) + (E.1.1), C1-5572: I.A + (H.2.7) + (E.1.1), C1-5573: I.A + (H.2.8) + (E.1.1), C1-5574:
I.A + (H.3.2) + (E.1.1), C1-5575: I.A + (H.3.4) + (E.1.1), C1-5576: I.A + (H.3.5) + (E.1.1), C1-
5577: I.A + (H.4.9) + (E.1.1), C1-5578: I.A + (H.4.10) + (E.1.1), C1-5579: I.A + (I.2.2) + (E.1.1),
C1-5580: I.A + (I.2.5) + (E.1.1), C1-5581: I.A + (J.1.2) + (E.1.1), C1-5582: I.A + (J.1.5) + (E.1.1),
C1-5583: I.A + (J.1.8) + (E.1.1), C1-5584: I.A + (J.1.11) + (E.1.1), C1-5585: I.A + (J.1.12) +
(E.1.1), C1-5586: I.A + (K.1.3) + (E.1.1), C1-5587: I.A + (K.1.29) + (E.1.1), C1-5588: I.A +
(K.1.30) + (E.1.1), C1-5589: I.A + (K.1.31) + (E.1.1), C1-5590: I.A + (K.1.32) + (E.1.1), C1-5591:
I.A + (K.1.41) + (E.1.1), C1-5592: I.A + (K.1.42) + (E.1.1), C1-5593: I.A + (K.1.44) + (E.1.1), C1-
5594: I.A + (K.1.45) + (E.1.1), C1-5595: I.A + (K.1.47) + (E.1.1), C1-5596: I.A + (K.1.49) +
(E.1.1), C1-5597: I.A + (K.1.53) + (E.1.1), C1-5598: I.A + (K.1.54) + (E.1.1), C1-5599: I.A +
(A.1.4) + (H.1.5), C1-5600: I.A + (A.1.12) + (H.1.5), C1-5601: I.A + (A.1.21) + (H.1.5), C1-5602:
I.A + (A.1.21a) + (H.1.5), C1-5603: I.A + (A.1.24) + (H.1.5), C1-5604: I.A + (A.1.26) + (H.1.5),
C1-5605: I.A + (A.1.27) + (H.1.5), C1-5606: I.A + (A.1.30) + (H.1.5), C1-5607: I.A + (A.1.31) +
(H.1.5), C1-5608: I.A + (A.1.32) + (H.1.5), C1-5609: I.A + (A.1.35) + (H.1.5), C1-5610: I.A +
(A.2.1) + (H.1.5), C1-5611: I.A + (A.2.3) + (H.1.5), C1-5612: I.A + (A.2.5) + (H.1.5), C1-5613: I.A +
(A.3.11) + (H.1.5), C1-5614: I.A + (A.3.12) + (H.1.5), C1-5615: I.A + (A.3.15) + (H.1.5), C1-
5616: I.A + (A.3.16) + (H.1.5), C1-5617: I.A + (A.3.18) + (H.1.5), C1-5618: I.A + (A.3.19) +
(H.1.5), C1-5619: I.A + (A.3.20) + (H.1.5), C1-5620: I.A + (A.3.21) + (H.1.5), C1-5621: I.A +
(A.3.22) + (H.1.5), C1-5622: I.A + (A.3.23) + (H.1.5), C1-5623: I.A + (A.3.25) + (H.1.5), C1-
5624: I.A + (A.3.26) + (H.1.5), C1-5625: I.A + (A.3.27) + (H.1.5), C1-5626: I.A + (A.4.5) +
(H.1.5), C1-5627: I.A + (A.4.11) + (H.1.5), C1-5628: I.A + (B.1.10) + (H.1.5), C1-5629: I.A +
(B.1.11) + (H.1.5), C1-5630: I.A + (B.1.12) + (H.1.5), C1-5631: I.A + (B.1.13) + (H.1.5), C1-
5632: I.A + (B.1.18) + (H.1.5), C1-5633: I.A + (B.1.21) + (H.1.5), C1-5634: I.A + (B.1.26) +
(H.1.5), C1-5635: I.A + (B.1.33) + (H.1.5), C1-5636: I.A + (B.1.34) + (H.1.5), C1-5637: I.A +
(B.1.37) + (H.1.5), C1-5638: I.A + (B.1.43) + (H.1.5), C1-5639: I.A + (B.1.46) + (H.1.5), C1-
5640: I.A + (B.2.5) + (H.1.5), C1-5641: I.A + (B.2.8) + (H.1.5), C1-5642: I.A + (C.1.1) + (H.1.5),
C1-5643: I.A + (C.1.2) + (H.1.5), C1-5644: I.A + (C.1.4) + (H.1.5), C1-5645: I.A + (C.1.5) +
(H.1.5), C1-5646: I.A + (C.2.6) + (H.1.5), C1-5647: I.A + (C.2.7) + (H.1.5), C1-5648: I.A +
(C.2.8) + (H.1.5), C1-5649: I.A + (D.1.1) + (H.1.5), C1-5650: I.A + (D.1.2) + (H.1.5), C1-5651:
I.A + (D.1.5) + (H.1.5), C1-5652: I.A + (D.2.4) + (H.1.5), C1-5653: I.A + (E.1.3) + (H.1.5), C1-
5654: I.A + (E.2.2) + (H.1.5), C1-5655: I.A + (E.2.3) + (H.1.5), C1-5656: I.A + (F.1.2) + (H.1.5),
C1-5657: I.A + (F.1.4) + (H.1.5), C1-5658: I.A + (F.1.5) + (H.1.5), C1-5659: I.A + (G.3.1) +
(H.1.5), C1-5660: I.A + (G.3.3) + (H.1.5), C1-5661: I.A + (G.3.6) + (H.1.5), C1-5662: I.A +
(G.5.1) + (H.1.5), C1-5663: I.A + (G.5.2) + (H.1.5), C1-5664: I.A + (G.5.3) + (H.1.5), C1-5665:
I.A + (G.5.4) + (H.1.5), C1-5666: I.A + (G.5.5) + (H.1.5), C1-5667: I.A + (G.5.6) + (H.1.5), C1-
5668: I.A + (G.5.7) + (H.1.5), C1-5669: I.A + (G.5.8) + (H.1.5), C1-5670: I.A + (G.5.9) + (H.1.5),
C1-5671: I.A + (G.5.10) + (H.1.5), C1-5672: I.A + (G.5.11) + (H.1.5), C1-5673: I.A + (H.1.7) +
(H.1.5), C1-5674: I.A + (H.2.2) + (H.1.5), C1-5675: I.A + (H.2.3) + (H.1.5), C1-5676: I.A +
(H.2.5) + (H.1.5), C1-5677: I.A + (H.2.7) + (H.1.5), C1-5678: I.A + (H.2.8) + (H.1.5), C1-5679:
I.A + (H.3.2) + (H.1.5), C1-5680: I.A + (H.3.4) + (H.1.5), C1-5681: I.A + (H.3.5) + (H.1.5), C1-
5682: I.A + (H.4.9) + (H.1.5), C1-5683: I.A + (H.4.10) + (H.1.5), C1-5684: I.A + (I.2.2) + (H.1.5),
C1-5685: I.A + (I.2.5) + (H.1.5), C1-5686: I.A + (J.1.2) + (H.1.5), C1-5687: I.A + (J.1.5) + (H.1.5),
C1-5688: I.A + (J.1.8) + (H.1.5), C1-5689: I.A + (J.1.11) + (H.1.5), C1-5690: I.A + (J.1.12) +
(H.1.5), C1-5691: I.A + (K.1.3) + (H.1.5), C1-5692: I.A + (K.1.29) + (H.1.5), C1-5693: I.A +
(K.1.30) + (H.1.5), C1-5694: I.A + (K.1.31) + (H.1.5), C1-5695: I.A + (K.1.32) + (H.1.5), C1-
5696: I.A + (K.1.41) + (H.1.5), C1-5697: I.A + (K.1.42) + (H.1.5), C1-5698: I.A + (K.1.44) +
(H.1.5), C1-5699: I.A + (K.1.45) + (H.1.5), C1-5700: I.A + (K.1.47) + (H.1.5), C1-5701: I.A +
(K.1.49) + (H.1.5), C1-5702: I.A + (K.1.53) + (H.1.5), C1-5703: I.A + (K.1.54) + (H.1.5), C1-
5704: I.A + (A.1.4) + (H.1.7), C1-5705: I.A + (A.1.12) + (H.1.7), C1-5706: I.A + (A.1.21) +
(H.1.7), C1-5707: I.A + (A.1,21a) + (H.1.7), C1-5708: I.A + (A.1.24) + (H.1.7), C1-5709: I.A +
(A.1.26) + (H.1.7), C1-5710: I.A + (A.1.27) + (H.1.7), C1-5711: I.A + (A.1.30) + (H.1.7), C1-
5712: I.A + (A.1.31) + (H.1.7), C1-5713: I.A + (A.1.32) + (H.1.7), C1-5714: I.A + (A.1.35) +
(H.1.7), C1-5715: I.A + (A.2.1) + (H.1.7), C1-5716: I.A + (A.2.3) + (H.1.7), C1-5717: I.A + (A.2.5) +
(H.1.7), C1-5718: I.A + (A.3.11) + (H.1.7), C1-5719: I.A + (A.3.12) + (H.1.7), C1-5720: I.A +
(A.3.15) + (H.1.7), C1-5721: I.A + (A.3.16) + (H.1.7), C1-5722: I.A + (A.3.18) + (H.1.7), C1-
5723: I.A + (A.3.19) + (H.1.7), C1-5724: I.A + (A.3.20) + (H.1.7), C1-5725: I.A + (A.3.21) +
(H.1.7), C1-5726: I.A + (A.3.22) + (H.1.7), C1-5727: I.A + (A.3.23) + (H.1.7), C1-5728: I.A +
(A.3.25) + (H.1.7), C1-5729: I.A + (A.3.26) + (H.1.7), C1-5730: I.A + (A.3.27) + (H.1.7), C1-
5731: I.A + (A.4.5) + (H.1.7), C1-5732: I.A + (A.4.11) + (H.1.7), C1-5733: I.A + (B.1.10) +
(H.1.7), C1-5734: I.A + (B.1.11) + (H.1.7), C1-5735: I.A + (B.1.12) + (H.1.7), C1-5736: I.A +
(B.1.13) + (H.1.7), C1-5737: I.A + (B.1.18) + (H.1.7), C1-5738: I.A + (B.1.21) + (H.1.7), C1-
5739: I.A + (B.1.26) + (H.1.7), C1-5740: I.A + (B.1.33) + (H.1.7), C1-5741: I.A + (B.1.34) +
(H.1.7), C1-5742: I.A + (B.1.37) + (H.1.7), C1-5743: I.A + (B.1.43) + (H.1.7), C1-5744: I.A +
(B.1.46) + (H.1.7), C1-5745: I.A + (B.2.5) + (H.1.7), C1-5746: I.A + (B.2.8) + (H.1.7), C1-5747:
I.A + (C.1.1) + (H.1.7), C1-5748: I.A + (C.1.2) + (H.1.7), C1-5749: I.A + (C.1.4) + (H.1.7), C1-
5750: I.A + (C.1.5) + (H.1.7), C1-5751: I.A + (C.2.6) + (H.1.7), C1-5752: I.A + (C.2.7) + (H.1.7),
C1-5753: I.A + (C.2.8) + (H.1.7), C1-5754: I.A + (D.1.1) + (H.1.7), C1-5755: I.A + (D.1.2) +
(H.1.7), C1-5756: I.A + (D.1.5) + (H.1.7), C1-5757: I.A + (D.2.4) + (H.1.7), C1-5758: I.A + (E.1.3) +
(H.1.7), C1-5759: I.A + (E.2.2) + (H.1.7), C1-5760: I.A + (E.2.3) + (H.1.7), C1-5761: I.A +
(F.1.2) + (H.1.7), C1-5762: I.A + (F.1.4) + (H.1.7), C1-5763: I.A + (F.1.5) + (H.1.7), C1-5764: I.A +
(G.3.1) + (H.1.7), C1-5765: I.A + (G.3.3) + (H.1.7), C1-5766: I.A + (G.3.6) + (H.1.7), C1-5767:
I.A + (G.5.1) + (H.1.7), C1-5768: I.A + (G.5.2) + (H.1.7), C1-5769: I.A + (G.5.3) + (H.1.7), C1-
5770: I.A + (G.5.4) + (H.1.7), C1-5771: I.A + (G.5.5) + (H.1.7), C1-5772: I.A + (G.5.6) + (H.1.7),
C1-5773: I.A + (G.5.7) + (H.1.7), C1-5774: I.A + (G.5.8) + (H.1.7), C1-5775: I.A + (G.5.9) +
(H.1.7), C1-5776: I.A + (G.5.10) + (H.1.7), C1-5777: I.A + (G.5.11) + (H.1.7), C1-5778: I.A +
(H.2.2) + (H.1.7), C1-5779: I.A + (H.2.3) + (H.1.7), C1-5780: I.A + (H.2.5) + (H.1.7), C1-5781:
I.A + (H.2.7) + (H.1.7), C1-5782: I.A + (H.2.8) + (H.1.7), C1-5783: I.A + (H.3.2) + (H.1.7), C1-
5784: I.A + (H.3.4) + (H.1.7), C1-5785: I.A + (H.3.5) + (H.1.7), C1-5786: I.A + (H.4.9) + (H.1.7),
C1-5787: I.A + (H.4.10) + (H.1.7), C1-5788: I.A + (I.2.2) + (H.1.7), C1-5789: I.A + (I.2.5) +
(H.1.7), C1-5790: I.A + (J.1.2) + (H.1.7), C1-5791: I.A + (J.1.5) + (H.1.7), C1-5792: I.A + (J.1.8) +
(H.1.7), C1-5793: I.A + (J.1.11) + (H.1.7), C1-5794: I.A + (J.1.12) + (H.1.7), C1-5795: I.A +
(K.1.3) + (H.1.7), C1-5796: I.A + (K.1.29) + (H.1.7), C1-5797: I.A + (K.1.30) + (H.1.7), C1-5798:
I.A + (K.1.31) + (H.1.7), C1-5799: I.A + (K.1.32) + (H.1.7), C1-5800: I.A + (K.1.41) + (H.1.7), C1-
5801: I.A + (K.1.42) + (H.1.7), C1-5802: I.A + (K.1.44) + (H.1.7), C1-5803: I.A + (K.1.45) +
(H.1.7), C1-5804: I.A + (K.1.47) + (H.1.7), C1-5805: I.A + (K.1.49) + (H.1.7), C1-5806: I.A +
(K.1.53) + (H.1.7), C1-5807: I.A + (K.1.54) + (H.1.7), C1-5808: I.A + (A.1.4) + (H.2.2), C1-5809:
I.A + (A.1.12) + (H.2.2), C1-5810: I.A + (A.1.21) + (H.2.2), C1-5811: I.A + (A.1.21a) + (H.2.2),
C1-5812: I.A + (A.1.24) + (H.2.2), C1-5813: I.A + (A.1.26) + (H.2.2), C1-5814: I.A + (A.1.27) +
(H.2.2), C1-5815: I.A + (A.1.30) + (H.2.2), C1-5816: I.A + (A.1.31) + (H.2.2), C1-5817: I.A +
(A.1.32) + (H.2.2), C1-5818: I.A + (A.1.35) + (H.2.2), C1-5819: I.A + (A.2.1) + (H.2.2), C1-5820:
I.A + (A.2.3) + (H.2.2), C1-5821: I.A + (A.2.5) + (H.2.2), C1-5822: I.A + (A.3.11) + (H.2.2), C1-
5823: I.A + (A.3.12) + (H.2.2), C1-5824: I.A + (A.3.15) + (H.2.2), C1-5825: I.A + (A.3.16) +
(H.2.2), C1-5826: I.A + (A.3.18) + (H.2.2), C1-5827: I.A + (A.3.19) + (H.2.2), C1-5828: I.A +
(A.3.20) + (H.2.2), C1-5829: I.A + (A.3.21) + (H.2.2), C1-5830: I.A + (A.3.22) + (H.2.2), C1-
5831: I.A + (A.3.23) + (H.2.2), C1-5832: I.A + (A.3.25) + (H.2.2), C1-5833: I.A + (A.3.26) +
(H.2.2), C1-5834: I.A + (A.3.27) + (H.2.2), C1-5835: I.A + (A.4.5) + (H.2.2), C1-5836: I.A +
(A.4.11) + (H.2.2), C1-5837: I.A + (B.1.10) + (H.2.2), C1-5838: I.A + (B.1.11) + (H.2.2), C1-
5839: I.A + (B.1.12) + (H.2.2), C1-5840: I.A + (B.1.13) + (H.2.2), C1-5841: I.A + (B.1.18) +
(H.2.2), C1-5842: I.A + (B.1.21) + (H.2.2), C1-5843: I.A + (B.1.26) + (H.2.2), C1-5844: I.A +
(B.1.33) + (H.2.2), C1-5845: I.A + (B.1.34) + (H.2.2), C1-5846: I.A + (B.1.37) + (H.2.2), C1-
5847: I.A + (B.1.43) + (H.2.2), C1-5848: I.A + (B.1.46) + (H.2.2), C1-5849: I.A + (B.2.5) +
(H.2.2), C1-5850: I.A + (B.2.8) + (H.2.2), C1-5851: I.A + (C.1.1) + (H.2.2), C1-5852: I.A + (C.1.2) +
(H.2.2), C1-5853: I.A + (C.1.4) + (H.2.2), C1-5854: I.A + (C.1.5) + (H.2.2), C1-5855: I.A +
(C.2.6) + (H.2.2), C1-5856: I.A + (C.2.7) + (H.2.2), C1-5857: I.A + (C.2.8) + (H.2.2), C1-5858:
I.A + (D.1.1) + (H.2.2), C1-5859: I.A + (D.1.2) + (H.2.2), C1-5860: I.A + (D.1.5) + (H.2.2), C1-
5861: I.A + (D.2.4) + (H.2.2), C1-5862: I.A + (E.1.3) + (H.2.2), C1-5863: I.A + (E.2.2) + (H.2.2),
C1-5864: I.A + (E.2.3) + (H.2.2), C1-5865: I.A + (F.1.2) + (H.2.2), C1-5866: I.A + (F.1.4) +
(H.2.2), C1-5867: I.A + (F.1.5) + (H.2.2), C1-5868: I.A + (G.3.1) + (H.2.2), C1-5869: I.A +
(G.3.3) + (H.2.2), C1-5870: I.A + (G.3.6) + (H.2.2), C1-5871: I.A + (G.5.1) + (H.2.2), C1-5872:
I.A + (G.5.2) + (H.2.2), C1-5873: I.A + (G.5.3) + (H.2.2), C1-5874: I.A + (G.5.4) + (H.2.2), C1-
5875: I.A + (G.5.5) + (H.2.2), C1-5876: I.A + (G.5.6) + (H.2.2), C1-5877: I.A + (G.5.7) + (H.2.2),
C1-5878: I.A + (G.5.8) + (H.2.2), C1-5879: I.A + (G.5.9) + (H.2.2), C1-5880: I.A + (G.5.10) +
(H.2.2), C1-5881: I.A + (G.5.11) + (H.2.2), C1-5882: I.A + (H.2.3) + (H.2.2), C1-5883: I.A +
(H.2.5) + (H.2.2), C1-5884: I.A + (H.2.7) + (H.2.2), C1-5885: I.A + (H.2.8) + (H.2.2), C1-5886:
I.A + (H.3.2) + (H.2.2), C1-5887: I.A + (H.3.4) + (H.2.2), C1-5888: I.A + (H.3.5) + (H.2.2), C1-
5889: I.A + (H.4.9) + (H.2.2), C1-5890: I.A + (H.4.10) + (H.2.2), C1-5891: I.A + (I.2.2) + (H.2.2),
C1-5892: I.A + (I.2.5) + (H.2.2), C1-5893: I.A + (J.1.2) + (H.2.2), C1-5894: I.A + (J.1.5) + (H.2.2),
C1-5895: I.A + (J.1.8) + (H.2.2), C1-5896: I.A + (J.1.11) + (H.2.2), C1-5897: I.A + (J.1.12) +
(H.2.2), C1-5898: I.A + (K.1.3) + (H.2.2), C1-5899: I.A + (K.1.29) + (H.2.2), C1-5900: I.A +
(K.1.30) + (H.2.2), C1-5901: I.A + (K.1.31) + (H.2.2), C1-5902: I.A + (K.1.32) + (H.2.2), C1-
5903: I.A + (K.1.41) + (H.2.2), C1-5904: I.A + (K.1.42) + (H.2.2), C1-5905: I.A + (K.1.44) +
(H.2.2), C1-5906: I.A + (K.1.45) + (H.2.2), C1-5907: I.A + (K.1.47) + (H.2.2), C1-5908: I.A +
(K.1.49) + (H.2.2), C1-5909: I.A + (K.1.53) + (H.2.2), C1-5910: I.A + (K.1.54) + (H.2.2), C1-
5911: I.A + (A.1.4) + (H.3.2), C1-5912: I.A + (A.1.12) + (H.3.2), C1-5913: I.A + (A.1.21) +
(H.3.2), C1-5914: I.A + (A.1,21a) + (H.3.2), C1-5915: I.A + (A.1.24) + (H.3.2), C1-5916: I.A +
(A.1.26) + (H.3.2), C1-5917: I.A + (A.1.27) + (H.3.2), C1-5918: I.A + (A.1.30) + (H.3.2), C1-
5919: I.A + (A.1.31) + (H.3.2), C1-5920: I.A + (A.1.32) + (H.3.2), C1-5921: I.A + (A.1.35) +
(H.3.2), C1-5922: I.A + (A.2.1) + (H.3.2), C1-5923: I.A + (A.2.3) + (H.3.2), C1-5924: I.A + (A.2.5) +
(H.3.2), C1-5925: I.A + (A.3.11) + (H.3.2), C1-5926: I.A + (A.3.12) + (H.3.2), C1-5927: I.A +
(A.3.15) + (H.3.2), C1-5928: I.A + (A.3.16) + (H.3.2), C1-5929: I.A + (A.3.18) + (H.3.2), C1-
5930: I.A + (A.3.19) + (H.3.2), C1-5931: I.A + (A.3.20) + (H.3.2), C1-5932: I.A + (A.3.21) +
(H.3.2), C1-5933: I.A + (A.3.22) + (H.3.2), C1-5934: I.A + (A.3.23) + (H.3.2), C1-5935: I.A +
(A.3.25) + (H.3.2), C1-5936: I.A + (A.3.26) + (H.3.2), C1-5937: I.A + (A.3.27) + (H.3.2), C1-
5938: I.A + (A.4.5) + (H.3.2), C1-5939: I.A + (A.4.11) + (H.3.2), C1-5940: I.A + (B.1.10) +
(H.3.2), C1-5941: I.A + (B.1.11) + (H.3.2), C1-5942: I.A + (B.1.12) + (H.3.2), C1-5943: I.A +
(B.1.13) + (H.3.2), C1-5944: I.A + (B.1.18) + (H.3.2), C1-5945: I.A + (B.1.21) + (H.3.2), C1-
5946: I.A + (B.1.26) + (H.3.2), C1-5947: I.A + (B.1.33) + (H.3.2), C1-5948: I.A + (B.1.34) +
(H.3.2), C1-5949: I.A + (B.1.37) + (H.3.2), C1-5950: I.A + (B.1.43) + (H.3.2), C1-5951: I.A +
(B.1.46) + (H.3.2), C1-5952: I.A + (B.2.5) + (H.3.2), C1-5953: I.A + (B.2.8) + (H.3.2), C1-5954:
I.A + (C.1.1) + (H.3.2), C1-5955: I.A + (C.1.2) + (H.3.2), C1-5956: I.A + (C.1.4) + (H.3.2), C1-
5957: I.A + (C.1.5) + (H.3.2), C1-5958: I.A + (C.2.6) + (H.3.2), C1-5959: I.A + (C.2.7) + (H.3.2),
C1-5960: I.A + (C.2.8) + (H.3.2), C1-5961: I.A + (D.1.1) + (H.3.2), C1-5962: I.A + (D.1.2) +
(H.3.2), C1-5963: I.A + (D.1.5) + (H.3.2), C1-5964: I.A + (D.2.4) + (H.3.2), C1-5965: I.A + (E.1.3) +
(H.3.2), C1-5966: I.A + (E.2.2) + (H.3.2), C1-5967: I.A + (E.2.3) + (H.3.2), C1-5968: I.A +
(F.1.2) + (H.3.2), C1-5969: I.A + (F.1.4) + (H.3.2), C1-5970: I.A + (F.1.5) + (H.3.2), C1-5971: I.A +
(G.3.1) + (H.3.2), C1-5972: I.A + (G.3.3) + (H.3.2), C1-5973: I.A + (G.3.6) + (H.3.2), C1-5974:
I.A + (G.5.1) + (H.3.2), C1-5975: I.A + (G.5.2) + (H.3.2), C1-5976: I.A + (G.5.3) + (H.3.2), C1-
5977: I.A + (G.5.4) + (H.3.2), C1-5978: I.A + (G.5.5) + (H.3.2), C1-5979: I.A + (G.5.6) + (H.3.2),
C1-5980: I.A + (G.5.7) + (H.3.2), C1-5981: I.A + (G.5.8) + (H.3.2), C1-5982: I.A + (G.5.9) +
(H.3.2), C1-5983: I.A + (G.5.10) + (H.3.2), C1-5984: I.A + (G.5.11) + (H.3.2), C1-5985: I.A +
(H.2.3) + (H.3.2), C1-5986: I.A + (H.2.5) + (H.3.2), C1-5987: I.A + (H.2.7) + (H.3.2), C1-5988:
I.A + (H.2.8) + (H.3.2), C1-5989: I.A + (H.3.4) + (H.3.2), C1-5990: I.A + (H.3.5) + (H.3.2), C1-
5991: I.A + (H.4.9) + (H.3.2), C1-5992: I.A + (H.4.10) + (H.3.2), C1-5993: I.A + (I.2.2) + (H.3.2),
C1-5994: I.A + (I.2.5) + (H.3.2), C1-5995: I.A + (J.1.2) + (H.3.2), C1-5996: I.A + (J.1.5) + (H.3.2),
C1-5997: I.A + (J.1.8) + (H.3.2), C1-5998: I.A + (J.1.11) + (H.3.2), C1-5999: I.A + (J.1.12) +
(H.3.2), C1-6000: I.A + (K.1.3) + (H.3.2), C1-6001: I.A + (K.1.29) + (H.3.2), C1-6002: I.A +
(K.1.30) + (H.3.2), C1-6003: I.A + (K.1.31) + (H.3.2), C1-6004: I.A + (K.1.32) + (H.3.2), C1-
6005: I.A + (K.1.41) + (H.3.2), C1-6006: I.A + (K.1.42) + (H.3.2), C1-6007: I.A + (K.1.44) +
(H.3.2), C1-6008: I.A + (K.1.45) + (H.3.2), C1-6009: I.A + (K.1.47) + (H.3.2), C1-6010: I.A +
(K.1.49) + (H.3.2), C1-6011: I.A + (K.1.53) + (H.3.2), C1-6012: I.A + (K.1.54) + (H.3.2), C1-
6013: I.A + (A.1.4) + (H.3.5), C1-6014: I.A + (A.1.12) + (H.3.5), C1-6015: I.A + (A.1.21) +
(H.3.5), C1-6016: I.A + (A.1,21a) + (H.3.5), C1-6017: I.A + (A.1.24) + (H.3.5), C1-6018: I.A +
(A.1.26) + (H.3.5), C1-6019: I.A + (A.1.27) + (H.3.5), C1-6020: I.A + (A.1.30) + (H.3.5), C1-
6021: I.A + (A.1.31) + (H.3.5), C1-6022: I.A + (A.1.32) + (H.3.5), C1-6023: I.A + (A.1.35) +
(H.3.5), C1-6024: I.A + (A.2.1) + (H.3.5), C1-6025: I.A + (A.2.3) + (H.3.5), C1-6026: I.A + (A.2.5) +
(H.3.5), C1-6027: I.A + (A.3.11) + (H.3.5), C1-6028: I.A + (A.3.12) + (H.3.5), C1-6029: I.A +
(A.3.15) + (H.3.5), C1-6030: I.A + (A.3.16) + (H.3.5), C1-6031: I.A + (A.3.18) + (H.3.5), C1-
6032: I.A + (A.3.19) + (H.3.5), C1-6033: I.A + (A.3.20) + (H.3.5), C1-6034: I.A + (A.3.21) +
(H.3.5), C1-6035: I.A + (A.3.22) + (H.3.5), C1-6036: I.A + (A.3.23) + (H.3.5), C1-6037: I.A +
(A.3.25) + (H.3.5), C1-6038: I.A + (A.3.26) + (H.3.5), C1-6039: I.A + (A.3.27) + (H.3.5), C1-
6040: I.A + (A.4.5) + (H.3.5), C1-6041: I.A + (A.4.11) + (H.3.5), C1-6042: I.A + (B.1.10) +
(H.3.5), C1-6043: I.A + (B.1.11) + (H.3.5), C1-6044: I.A + (B.1.12) + (H.3.5), C1-6045: I.A +
(B.1.13) + (H.3.5), C1-6046: I.A + (B.1.18) + (H.3.5), C1-6047: I.A + (B.1.21) + (H.3.5), C1-
6048: I.A + (B.1.26) + (H.3.5), C1-6049: I.A + (B.1.33) + (H.3.5), C1-6050: I.A + (B.1.34) +
(H.3.5), C1-6051: I.A + (B.1.37) + (H.3.5), C1-6052: I.A + (B.1.43) + (H.3.5), C1-6053: I.A +
(B.1.46) + (H.3.5), C1-6054: I.A + (B.2.5) + (H.3.5), C1-6055: I.A + (B.2.8) + (H.3.5), C1-6056:
I.A + (C.1.1) + (H.3.5), C1-6057: I.A + (C.1.2) + (H.3.5), C1-6058: I.A + (C.1.4) + (H.3.5), C1-
6059: I.A + (C.1.5) + (H.3.5), C1-6060: I.A + (C.2.6) + (H.3.5), C1-6061: I.A + (C.2.7) + (H.3.5),
C1-6062: I.A + (C.2.8) + (H.3.5), C1-6063: I.A + (D.1.1) + (H.3.5), C1-6064: I.A + (D.1.2) +
(H.3.5), C1-6065: I.A + (D.1.5) + (H.3.5), C1-6066: I.A + (D.2.4) + (H.3.5), C1-6067: I.A + (E.1.3) +
(H.3.5), C1-6068: I.A + (E.2.2) + (H.3.5), C1-6069: I.A + (E.2.3) + (H.3.5), C1-6070: I.A +
(F.1.2) + (H.3.5), C1-6071: I.A + (F.1.4) + (H.3.5), C1-6072: I.A + (F.1.5) + (H.3.5), C1-6073: I.A +
(G.3.1) + (H.3.5), C1-6074: I.A + (G.3.3) + (H.3.5), C1-6075: I.A + (G.3.6) + (H.3.5), C1-6076:
I.A + (G.5.1) + (H.3.5), C1-6077: I.A + (G.5.2) + (H.3.5), C1-6078: I.A + (G.5.3) + (H.3.5), C1-
6079: I.A + (G.5.4) + (H.3.5), C1-6080: I.A + (G.5.5) + (H.3.5), C1-6081: I.A + (G.5.6) + (H.3.5),
C1-6082: I.A + (G.5.7) + (H.3.5), C1-6083: I.A + (G.5.8) + (H.3.5), C1-6084: I.A + (G.5.9) +
(H.3.5), C1-6085: I.A + (G.5.10) + (H.3.5), C1-6086: I.A + (G.5.11) + (H.3.5), C1-6087: I.A +
(H.2.3) + (H.3.5), C1-6088: I.A + (H.2.5) + (H.3.5), C1-6089: I.A + (H.2.7) + (H.3.5), C1-6090:
I.A + (H.2.8) + (H.3.5), C1-6091: I.A + (H.3.4) + (H.3.5), C1-6092: I.A + (H.4.9) + (H.3.5), C1-
6093: I.A + (H.4.10) + (H.3.5), C1-6094: I.A + (I.2.2) + (H.3.5), C1-6095: I.A + (I.2.5) + (H.3.5),
C1-6096: I.A + (J.1.2) + (H.3.5), C1-6097: I.A + (J.1.5) + (H.3.5), C1-6098: I.A + (J.1.8) +
(H.3.5), C1-6099: I.A + (J.1.11) + (H.3.5), C1-6100: I.A + (J.1.12) + (H.3.5), C1-6101: I.A +
(K.1.3) + (H.3.5), C1-6102: I.A + (K.1.29) + (H.3.5), C1-6103: I.A + (K.1.30) + (H.3.5), C1-6104:
I.A + (K.1.31) + (H.3.5), C1-6105: I.A + (K.1.32) + (H.3.5), C1-6106: I.A + (K.1.41) + (H.3.5), C1-
6107: I.A + (K.1.42) + (H.3.5), C1-6108: I.A + (K.1.44) + (H.3.5), C1-6109: I.A + (K.1.45) +
(H.3.5), C1-6110: I.A + (K.1.47) + (H.3.5), C1-6111: I.A + (K.1.49) + (H.3.5), C1-6112: I.A +
(K.1.53) + (H.3.5), C1-6113: I.A + (K.1.54) + (H.3.5), C1-6114: I.A + (A.1.4) + (K.1.3), C1-6115:
I.A + (A.1.12) + (K.1.3), C1-6116: I.A + (A.1.21) + (K.1.3), C1-6117: I.A + (A.1.21a) + (K.1.3),
C1-6118: I.A + (A.1.24) + (K.1.3), C1-6119: I.A + (A.1.26) + (K.1.3), C1-6120: I.A + (A.1.27) +
(K.1.3), C1-6121: I.A + (A.1.30) + (K.1.3), C1-6122: I.A + (A.1.31) + (K.1.3), C1-6123: I.A +
(A.1.32) + (K.1.3), C1-6124: I.A + (A.1.35) + (K.1.3), C1-6125: I.A + (A.2.1) + (K.1.3), C1-6126:
I.A + (A.2.3) + (K.1.3), C1-6127: I.A + (A.2.5) + (K.1.3), C1-6128: I.A + (A.3.11) + (K.1.3), C1-
6129: I.A + (A.3.12) + (K.1.3), C1-6130: I.A + (A.3.15) + (K.1.3), C1-6131: I.A + (A.3.16) +
(K.1.3), C1-6132: I.A + (A.3.18) + (K.1.3), C1-6133: I.A + (A.3.19) + (K.1.3), C1-6134: I.A +
(A.3.20) + (K.1.3), C1-6135: I.A + (A.3.21) + (K.1.3), C1-6136: I.A + (A.3.22) + (K.1.3), C1-6137:
I.A + (A.3.23) + (K.1.3), C1-6138: I.A + (A.3.25) + (K.1.3), C1-6139: I.A + (A.3.26) + (K.1.3), C1-
6140: I.A + (A.3.27) + (K.1.3), C1-6141: I.A + (A.4.5) + (K.1.3), C1-6142: I.A + (A.4.11) +
(K.1.3), C1-6143: I.A + (B.1.10) + (K.1.3), C1-6144: I.A + (B.1.11) + (K.1.3), C1-6145: I.A +
(B.1.12) + (K.1.3), C1-6146: I.A + (B.1.13) + (K.1.3), C1-6147: I.A + (B.1.18) + (K.1.3), C1-6148:
I.A + (B.1.21) + (K.1.3), C1-6149: I.A + (B.1.26) + (K.1.3), C1-6150: I.A + (B.1.33) + (K.1.3), C1-
6151: I.A + (B.1.34) + (K.1.3), C1-6152: I.A + (B.1.37) + (K.1.3), C1-6153: I.A + (B.1.43) +
(K.1.3), C1-6154: I.A + (B.1.46) + (K.1.3), C1-6155: I.A + (B.2.5) + (K.1.3), C1-6156: I.A +
(B.2.8) + (K.1.3), C1-6157: I.A + (C.1.1) + (K.1.3), C1-6158: I.A + (C.1.2) + (K.1.3), C1-6159: I.A +
(C.1.4) + (K.1.3), C1-6160: I.A + (C.1.5) + (K.1.3), C1-6161: I.A + (C.2.6) + (K.1.3), C1-6162:
I.A + (C.2.7) + (K.1.3), C1-6163: I.A + (C.2.8) + (K.1.3), C1-6164: I.A + (D.1.1) + (K.1.3), C1-
6165: I.A + (D.1.2) + (K.1.3), C1-6166: I.A + (D.1.5) + (K.1.3), C1-6167: I.A + (D.2.4) + (K.1.3),
C1-6168: I.A + (E.1.3) + (K.1.3), C1-6169: I.A + (E.2.2) + (K.1.3), C1-6170: I.A + (E.2.3) +
(K.1.3), C1-6171: I.A + (F.1.2) + (K.1.3), C1-6172: I.A + (F.1.4) + (K.1.3), C1-6173: I.A + (F.1.5) +
(K.1.3), C1-6174: I.A + (G.3.1) + (K.1.3), C1-6175: I.A + (G.3.3) + (K.1.3), C1-6176: I.A +
(G.3.6) + (K.1.3), C1-6177: I.A + (G.5.1) + (K.1.3), C1-6178: I.A + (G.5.2) + (K.1.3), C1-6179:
I.A + (G.5.3) + (K.1.3), C1-6180: I.A + (G.5.4) + (K.1.3), C1-6181: I.A + (G.5.5) + (K.1.3), C1-
6182: I.A + (G.5.6) + (K.1.3), C1-6183: I.A + (G.5.7) + (K.1.3), C1-6184: I.A + (G.5.8) + (K.1.3),
C1-6185: I.A + (G.5.9) + (K.1.3), C1-6186: I.A + (G.5.10) + (K.1.3), C1-6187: I.A + (G.5.11) +
(K.1.3), C1-6188: I.A + (H.2.3) + (K.1.3), C1-6189: I.A + (H.2.5) + (K.1.3), C1-6190: I.A + (H.2.7) +
(K.1.3), C1-6191: I.A + (H.2.8) + (K.1.3), C1-6192: I.A + (H.3.4) + (K.1.3), C1-6193: I.A +
(H.4.9) + (K.1.3), C1-6194: I.A + (H.4.10) + (K.1.3), C1-6195: I.A + (I.2.2) + (K.1.3), C1-6196: I.A +
(I.2.5) + (K.1.3), C1-6197: I.A + (J.1.2) + (K.1.3), C1-6198: I.A + (J.1.5) + (K.1.3), C1-6199: I.A +
(J.1.8) + (K.1.3), C1-6200: I.A + (J.1.11) + (K.1.3), C1-6201: I.A + (J.1.12) + (K.1.3), C1-6202:
I.A + (K.1.3) + (K.1.3), C1-6203: I.A + (K.1.29) + (K.1.3), C1-6204: I.A + (K.1.30) + (K.1.3), C1-
6205: I.A + (K.1.31) + (K.1.3), C1-6206: I.A + (K.1.32) + (K.1.3), C1-6207: I.A + (K.1.41) +
(K.1.3), C1-6208: I.A + (K.1.42) + (K.1.3), C1-6209: I.A + (K.1.44) + (K.1.3), C1-6210: I.A +
(K.1.45) + (K.1.3), C1-6211: I.A + (K.1.47) + (K.1.3), C1-6212: I.A + (K.1.49) + (K.1.3), C1-6213:
I.A + (K.1.53) + (K.1.3), C1-6214: I.A + (K.1.54) + (K.1.3), C1-6215: I.A + (A.1.4) + (K.1.29), C1-
6216: I.A + (A.1.12) + (K.1.29), C1-6217: I.A + (A.1.21) + (K.1.29), C1-6218: I.A + (A.1,21a) +
(K.1.29), C1-6219: I.A + (A.1.24) + (K.1.29), C1-6220: I.A + (A.1.26) + (K.1.29), C1-6221: I.A +
(A.1.27) + (K.1.29), C1-6222: I.A + (A.1.30) + (K.1.29), C1-6223: I.A + (A.1.31) + (K.1.29), C1-
6224: I.A + (A.1.32) + (K.1.29), C1-6225: I.A + (A.1.35) + (K.1.29), C1-6226: I.A + (A.2.1) +
(K.1.29), C1-6227: I.A + (A.2.3) + (K.1.29), C1-6228: I.A + (A.2.5) + (K.1.29), C1-6229: I.A +
(A.3.11) + (K.1.29), C1-6230: I.A + (A.3.12) + (K.1.29), C1-6231: I.A + (A.3.15) + (K.1.29), C1-
6232: I.A + (A.3.16) + (K.1.29), C1-6233: I.A + (A.3.18) + (K.1.29), C1-6234: I.A + (A.3.19) +
(K.1.29), C1-6235: I.A + (A.3.20) + (K.1.29), C1-6236: I.A + (A.3.21) + (K.1.29), C1-6237: I.A +
(A.3.22) + (K.1.29), C1-6238: I.A + (A.3.23) + (K.1.29), C1-6239: I.A + (A.3.25) + (K.1.29), C1-
6240: I.A + (A.3.26) + (K.1.29), C1-6241: I.A + (A.3.27) + (K.1.29), C1-6242: I.A + (A.4.5) +
(K.1.29), C1-6243: I.A + (A.4.11) + (K.1.29), C1-6244: I.A + (B.1.10) + (K.1.29), C1-6245: I.A +
(B.1.11) + (K.1.29), C1-6246: I.A + (B.1.12) + (K.1.29), C1-6247: I.A + (B.1.13) + (K.1.29), C1-
6248: I.A + (B.1.18) + (K.1.29), C1-6249: I.A + (B.1.21) + (K.1.29), C1-6250: I.A + (B.1.26) +
(K.1.29), C1-6251: I.A + (B.1.33) + (K.1.29), C1-6252: I.A + (B.1.34) + (K.1.29), C1-6253: I.A +
(B.1.37) + (K.1.29), C1-6254: I.A + (B.1.43) + (K.1.29), C1-6255: I.A + (B.1.46) + (K.1.29), C1-
6256: I.A + (B.2.5) + (K.1.29), C1-6257: I.A + (B.2.8) + (K.1.29), C1-6258: I.A + (C.1.1) +
(K.1.29), C1-6259: I.A + (C.1.2) + (K.1.29), C1-6260: I.A + (C.1.4) + (K.1.29), C1-6261: I.A +
(C.1.5) + (K.1.29), C1-6262: I.A + (C.2.6) + (K.1.29), C1-6263: I.A + (C.2.7) + (K.1.29), C1-
6264: I.A + (C.2.8) + (K.1.29), C1-6265: I.A + (D.1.1) + (K.1.29), C1-6266: I.A + (D.1.2) +
(K.1.29), C1-6267: I.A + (D.1.5) + (K.1.29), C1-6268: I.A + (D.2.4) + (K.1.29), C1-6269: I.A +
(E.1.3) + (K.1.29), C1-6270: I.A + (E.2.2) + (K.1.29), C1-6271: I.A + (E.2.3) + (K.1.29), C1-6272:
I.A + (F.1.2) + (K.1.29), C1-6273: I.A + (F.1.4) + (K.1.29), C1-6274: I.A + (F.1.5) + (K.1.29), C1-
6275: I.A + (G.3.1) + (K.1.29), C1-6276: I.A + (G.3.3) + (K.1.29), C1-6277: I.A + (G.3.6) +
(K.1.29), C1-6278: I.A + (G.5.1) + (K.1.29), C1-6279: I.A + (G.5.2) + (K.1.29), C1-6280: I.A +
(G.5.3) + (K.1.29), C1-6281: I.A + (G.5.4) + (K.1.29), C1-6282: I.A + (G.5.5) + (K.1.29), C1-
6283: I.A + (G.5.6) + (K.1.29), C1-6284: I.A + (G.5.7) + (K.1.29), C1-6285: I.A + (G.5.8) +
(K.1.29), C1-6286: I.A + (G.5.9) + (K.1.29), C1-6287: I.A + (G.5.10) + (K.1.29), C1-6288: I.A +
(G.5.11) + (K.1.29), C1-6289: I.A + (H.2.3) + (K.1.29), C1-6290: I.A + (H.2.5) + (K.1.29), C1-
6291: I.A + (H.2.7) + (K.1.29), C1-6292: I.A + (H.2.8) + (K.1.29), C1-6293: I.A + (H.3.4) +
(K.1.29), C1-6294: I.A + (H.4.9) + (K.1.29), C1-6295: I.A + (H.4.10) + (K.1.29), C1-6296: I.A +
(I.2.2) + (K.1.29), C1-6297: I.A + (I.2.5) + (K.1.29), C1-6298: I.A + (J.1.2) + (K.1.29), C1-6299:
I.A + (J.1.5) + (K.1.29), C1-6300: I.A + (J.1.8) + (K.1.29), C1-6301: I.A + (J.1.11) + (K.1.29), C1-
6302: I.A + (J.1.12) + (K.1.29), C1-6303: I.A + (K.1.41) + (K.1.29), C1-6304: I.A + (K.1.44) +
(K.1.29), C1-6305: I.A + (K.1.45) + (K.1.29), C1-6306: I.A + (K.1.47) + (K.1.29), C1-6307: I.A +
(K.1.49) + (K.1.29), C1-6308: I.A + (A.1.4) + (K.1.30), C1-6309: I.A + (A.1.12) + (K.1.30), C1-
6310: I.A + (A.1.21) + (K.1.30), C1-6311: I.A + (A.1.21a) + (K.1.30), C1-6312: I.A + (A.1.24) +
(K.1.30), C1-6313: I.A + (A.1.26) + (K.1.30), C1-6314: I.A + (A.1.27) + (K.1.30), C1-6315: I.A +
(A.1.30) + (K.1.30), C1-6316: I.A + (A.1.31) + (K.1.30), C1-6317: I.A + (A.1.32) + (K.1.30), C1-
6318: I.A + (A.1.35) + (K.1.30), C1-6319: I.A + (A.2.1) + (K.1.30), C1-6320: I.A + (A.2.3) +
(K.1.30), C1-6321: I.A + (A.2.5) + (K.1.30), C1-6322: I.A + (A.3.11) + (K.1.30), C1-6323: I.A +
(A.3.12) + (K.1.30), C1-6324: I.A + (A.3.15) + (K.1.30), C1-6325: I.A + (A.3.16) + (K.1.30), C1-
6326: I.A + (A.3.18) + (K.1.30), C1-6327: I.A + (A.3.19) + (K.1.30), C1-6328: I.A + (A.3.20) +
(K.1.30), C1-6329: I.A + (A.3.21) + (K.1.30), C1-6330: I.A + (A.3.22) + (K.1.30), C1-6331: I.A +
(A.3.23) + (K.1.30), C1-6332: I.A + (A.3.25) + (K.1.30), C1-6333: I.A + (A.3.26) + (K.1.30), C1-
6334: I.A + (A.3.27) + (K.1.30), C1-6335: I.A + (A.4.5) + (K.1.30), C1-6336: I.A + (A.4.11) +
(K.1.30), C1-6337: I.A + (B.1.10) + (K.1.30), C1-6338: I.A + (B.1.11) + (K.1.30), C1-6339: I.A +
(B.1.12) + (K.1.30), C1-6340: I.A + (B.1.13) + (K.1.30), C1-6341: I.A + (B.1.18) + (K.1.30), C1-
6342: I.A + (B.1.21) + (K.1.30), C1-6343: I.A + (B.1.26) + (K.1.30), C1-6344: I.A + (B.1.33) +
(K.1.30), C1-6345: I.A + (B.1.34) + (K.1.30), C1-6346: I.A + (B.1.37) + (K.1.30), C1-6347: I.A +
(B.1.43) + (K.1.30), C1-6348: I.A + (B.1.46) + (K.1.30), C1-6349: I.A + (B.2.5) + (K.1.30), C1-
6350: I.A + (B.2.8) + (K.1.30), C1-6351: I.A + (C.1.1) + (K.1.30), C1-6352: I.A + (C.1.2) +
(K.1.30), C1-6353: I.A + (C.1.4) + (K.1.30), C1-6354: I.A + (C.1.5) + (K.1.30), C1-6355: I.A +
(C.2.6) + (K.1.30), C1-6356: I.A + (C.2.7) + (K.1.30), C1-6357: I.A + (C.2.8) + (K.1.30), C1-
6358: I.A + (D.1.1) + (K.1.30), C1-6359: I.A + (D.1.2) + (K.1.30), C1-6360: I.A + (D.1.5) +
(K.1.30), C1-6361: I.A + (D.2.4) + (K.1.30), C1-6362: I.A + (E.1.3) + (K.1.30), C1-6363: I.A +
(E.2.2) + (K.1.30), C1-6364: I.A + (E.2.3) + (K.1.30), C1-6365: I.A + (F.1.2) + (K.1.30), C1-6366:
I.A + (F.1.4) + (K.1.30), C1-6367: I.A + (F.1.5) + (K.1.30), C1-6368: I.A + (G.3.1) + (K.1.30), C1-
6369: I.A + (G.3.3) + (K.1.30), C1-6370: I.A + (G.3.6) + (K.1.30), C1-6371: I.A + (G.5.1) +
(K.1.30), C1-6372: I.A + (G.5.2) + (K.1.30), C1-6373: I.A + (G.5.3) + (K.1.30), C1-6374: I.A +
(G.5.4) + (K.1.30), C1-6375: I.A + (G.5.5) + (K.1.30), C1-6376: I.A + (G.5.6) + (K.1.30), C1-
6377: I.A + (G.5.7) + (K.1.30), C1-6378: I.A + (G.5.8) + (K.1.30), C1-6379: I.A + (G.5.9) +
(K.1.30), C1-6380: I.A + (G.5.10) + (K.1.30), C1-6381: I.A + (G.5.11) + (K.1.30), C1-6382: I.A +
(H.2.3) + (K.1.30), C1-6383: I.A + (H.2.5) + (K.1.30), C1-6384: I.A + (H.2.7) + (K.1.30), C1-
6385: I.A + (H.2.8) + (K.1.30), C1-6386: I.A + (H.3.4) + (K.1.30), C1-6387: I.A + (H.4.9) +
(K.1.30), C1-6388: I.A + (H.4.10) + (K.1.30), C1-6389: I.A + (I.2.2) + (K.1.30), C1-6390: I.A +
(I.2.5) + (K.1.30), C1-6391: I.A + (J.1.2) + (K.1.30), C1-6392: I.A + (J.1.5) + (K.1.30), C1-6393:
I.A + (J.1.8) + (K.1.30), C1-6394: I.A + (J.1.11) + (K.1.30), C1-6395: I.A + (J.1.12) + (K.1.30),
C1-6396: I.A + (K.1.41) + (K.1.30), C1-6397: I.A + (K.1.44) + (K.1.30), C1-6398: I.A + (K.1.45) +
(K.1.30), C1-6399: I.A + (K.1.47) + (K.1.30), C1-6400: I.A + (K.1.49) + (K.1.30), C1-6401: I.A +
(A.1.4) + (K.1.31), C1-6402: I.A + (A.1.12) + (K.1.31), C1-6403: I.A + (A.1.21) + (K.1.31), C1-
6404: I.A + (A.1.21a) + (K.1.31), C1-6405: I.A + (A.1.24) + (K.1.31), C1-6406: I.A + (A.1.26) +
(K.1.31), C1-6407: I.A + (A.1.27) + (K.1.31), C1-6408: I.A + (A.1.30) + (K.1.31), C1-6409: I.A +
(A.1.31) + (K.1.31), C1-6410: I.A + (A.1.32) + (K.1.31), C1-6411: I.A + (A.1.35) + (K.1.31), C1-
6412: I.A + (A.2.1) + (K.1.31), C1-6413: I.A + (A.2.3) + (K.1.31), C1-6414: I.A + (A.2.5) +
(K.1.31), C1-6415: I.A + (A.3.11) + (K.1.31), C1-6416: I.A + (A.3.12) + (K.1.31), C1-6417: I.A +
(A.3.15) + (K.1.31), C1-6418: I.A + (A.3.16) + (K.1.31), C1-6419: I.A + (A.3.18) + (K.1.31), C1-
6420: I.A + (A.3.19) + (K.1.31), C1-6421: I.A + (A.3.20) + (K.1.31), C1-6422: I.A + (A.3.21) +
(K.1.31), C1-6423: I.A + (A.3.22) + (K.1.31), C1-6424: I.A + (A.3.23) + (K.1.31), C1-6425: I.A +
(A.3.25) + (K.1.31), C1-6426: I.A + (A.3.26) + (K.1.31), C1-6427: I.A + (A.3.27) + (K.1.31), C1-
6428: I.A + (A.4.5) + (K.1.31), C1-6429: I.A + (A.4.11) + (K.1.31), C1-6430: I.A + (B.1.10) +
(K.1.31), C1-6431: I.A + (B.1.11) + (K.1.31), C1-6432: I.A + (B.1.12) + (K.1.31), C1-6433: I.A +
(B.1.13) + (K.1.31), C1-6434: I.A + (B.1.18) + (K.1.31), C1-6435: I.A + (B.1.21) + (K.1.31), C1-
6436: I.A + (B.1.26) + (K.1.31), C1-6437: I.A + (B.1.33) + (K.1.31), C1-6438: I.A + (B.1.34) +
(K.1.31), C1-6439: I.A + (B.1.37) + (K.1.31), C1-6440: I.A + (B.1.43) + (K.1.31), C1-6441: I.A +
(B.1.46) + (K.1.31), C1-6442: I.A + (B.2.5) + (K.1.31), C1-6443: I.A + (B.2.8) + (K.1.31), C1-
6444: I.A + (C.1.1) + (K.1.31), C1-6445: I.A + (C.1.2) + (K.1.31), C1-6446: I.A + (C.1.4) +
(K.1.31), C1-6447: I.A + (C.1.5) + (K.1.31), C1-6448: I.A + (C.2.6) + (K.1.31), C1-6449: I.A +
(C.2.7) + (K.1.31), C1-6450: I.A + (C.2.8) + (K.1.31), C1-6451: I.A + (D.1.1) + (K.1.31), C1-
6452: I.A + (D.1.2) + (K.1.31), C1-6453: I.A + (D.1.5) + (K.1.31), C1-6454: I.A + (D.2.4) +
(K.1.31), C1-6455: I.A + (E.1.3) + (K.1.31), C1-6456: I.A + (E.2.2) + (K.1.31), C1-6457: I.A +
(E.2.3) + (K.1.31), C1-6458: I.A + (F.1.2) + (K.1.31), C1-6459: I.A + (F.1.4) + (K.1.31), C1-6460:
I.A + (F.1.5) + (K.1.31), C1-6461: I.A + (G.3.1) + (K.1.31), C1-6462: I.A + (G.3.3) + (K.1.31), C1-
6463: I.A + (G.3.6) + (K.1.31), C1-6464: I.A + (G.5.1) + (K.1.31), C1-6465: I.A + (G.5.2) +
(K.1.31), C1-6466: I.A + (G.5.3) + (K.1.31), C1-6467: I.A + (G.5.4) + (K.1.31), C1-6468: I.A +
(G.5.5) + (K.1.31), C1-6469: I.A + (G.5.6) + (K.1.31), C1-6470: I.A + (G.5.7) + (K.1.31), C1-
6471: I.A + (G.5.8) + (K.1.31), C1-6472: I.A + (G.5.9) + (K.1.31), C1-6473: I.A + (G.5.10) +
(K.1.31), C1-6474: I.A + (G.5.11) + (K.1.31), C1-6475: I.A + (H.2.3) + (K.1.31), C1-6476: I.A +
(H.2.5) + (K.1.31), C1-6477: I.A + (H.2.7) + (K.1.31), C1-6478: I.A + (H.2.8) + (K.1.31), C1-
6479: I.A + (H.3.4) + (K.1.31), C1-6480: I.A + (H.4.9) + (K.1.31), C1-6481: I.A + (H.4.10) +
(K.1.31), C1-6482: I.A + (I.2.2) + (K.1.31), C1-6483: I.A + (I.2.5) + (K.1.31), C1-6484: I.A +
(J.1.2) + (K.1.31), C1-6485: I.A + (J.1.5) + (K.1.31), C1-6486: I.A + (J.1.8) + (K.1.31), C1-6487:
I.A + (J.1.11) + (K.1.31), C1-6488: I.A + (J.1.12) + (K.1.31), C1-6489: I.A + (K.1.41) + (K.1.31),
C1-6490: I.A + (K.1.44) + (K.1.31), C1-6491: I.A + (K.1.45) + (K.1.31), C1-6492: I.A + (K.1.47) +
(K.1.31), C1-6493: I.A + (K.1.49) + (K.1.31), C1-6494: I.A + (A.1.4) + (K.1.32), C1-6495: I.A +
(A.1.12) + (K.1.32), C1-6496: I.A + (A.1.21) + (K.1.32), C1-6497: I.A + (A.1.21a) + (K.1.32), C1-
6498: I.A + (A.1.24) + (K.1.32), C1-6499: I.A + (A.1.26) + (K.1.32), C1-6500: I.A + (A.1.27) +
(K.1.32), C1-6501: I.A + (A.1.30) + (K.1.32), C1-6502: I.A + (A.1.31) + (K.1.32), C1-6503: I.A +
(A.1.32) + (K.1.32), C1-6504: I.A + (A.1.35) + (K.1.32), C1-6505: I.A + (A.2.1) + (K.1.32), C1-
6506: I.A + (A.2.3) + (K.1.32), C1-6507: I.A + (A.2.5) + (K.1.32), C1-6508: I.A + (A.3.11) +
(K.1.32), C1-6509: I.A + (A.3.12) + (K.1.32), C1-6510: I.A + (A.3.15) + (K.1.32), C1-6511: I.A +
(A.3.16) + (K.1.32), C1-6512: I.A + (A.3.18) + (K.1.32), C1-6513: I.A + (A.3.19) + (K.1.32), C1-
6514: I.A + (A.3.20) + (K.1.32), C1-6515: I.A + (A.3.21) + (K.1.32), C1-6516: I.A + (A.3.22) +
(K.1.32), C1-6517: I.A + (A.3.23) + (K.1.32), C1-6518: I.A + (A.3.25) + (K.1.32), C1-6519: I.A +
(A.3.26) + (K.1.32), C1-6520: I.A + (A.3.27) + (K.1.32), C1-6521: I.A + (A.4.5) + (K.1.32), C1-
6522: I.A + (A.4.11) + (K.1.32), C1-6523: I.A + (B.1.10) + (K.1.32), C1-6524: I.A + (B.1.11) +
(K.1.32), C1-6525: I.A + (B.1.12) + (K.1.32), C1-6526: I.A + (B.1.13) + (K.1.32), C1-6527: I.A +
(B.1.18) + (K.1.32), C1-6528: I.A + (B.1.21) + (K.1.32), C1-6529: I.A + (B.1.26) + (K.1.32), C1-
6530: I.A + (B.1.33) + (K.1.32), C1-6531: I.A + (B.1.34) + (K.1.32), C1-6532: I.A + (B.1.37) +
(K.1.32), C1-6533: I.A + (B.1.43) + (K.1.32), C1-6534: I.A + (B.1.46) + (K.1.32), C1-6535: I.A +
(B.2.5) + (K.1.32), C1-6536: I.A + (B.2.8) + (K.1.32), C1-6537: I.A + (C.1.1) + (K.1.32), C1-6538:
I.A + (C.1.2) + (K.1.32), C1-6539: I.A + (C.1.4) + (K.1.32), C1-6540: I.A + (C.1.5) + (K.1.32), C1-
6541: I.A + (C.2.6) + (K.1.32), C1-6542: I.A + (C.2.7) + (K.1.32), C1-6543: I.A + (C.2.8) +
(K.1.32), C1-6544: I.A + (D.1.1) + (K.1.32), C1-6545: I.A + (D.1.2) + (K.1.32), C1-6546: I.A +
(D.1.5) + (K.1.32), C1-6547: I.A + (D.2.4) + (K.1.32), C1-6548: I.A + (E.1.3) + (K.1.32), C1-6549:
I.A + (E.2.2) + (K.1.32), C1-6550: I.A + (E.2.3) + (K.1.32), C1-6551: I.A + (F.1.2) + (K.1.32), C1-
6552: I.A + (F.1.4) + (K.1.32), C1-6553: I.A + (F.1.5) + (K.1.32), C1-6554: I.A + (G.3.1) +
(K.1.32), C1-6555: I.A + (G.3.3) + (K.1.32), C1-6556: I.A + (G.3.6) + (K.1.32), C1-6557: I.A +
(G.5.1) + (K.1.32), C1-6558: I.A + (G.5.2) + (K.1.32), C1-6559: I.A + (G.5.3) + (K.1.32), C1-
6560: I.A + (G.5.4) + (K.1.32), C1-6561: I.A + (G.5.5) + (K.1.32), C1-6562: I.A + (G.5.6) +
(K.1.32), C1-6563: I.A + (G.5.7) + (K.1.32), C1-6564: I.A + (G.5.8) + (K.1.32), C1-6565: I.A +
(G.5.9) + (K.1.32), C1-6566: I.A + (G.5.10) + (K.1.32), C1-6567: I.A + (G.5.11) + (K.1.32), C1-
6568: I.A + (H.2.3) + (K.1.32), C1-6569: I.A + (H.2.5) + (K.1.32), C1-6570: I.A + (H.2.7) +
(K.1.32), C1-6571: I.A + (H.2.8) + (K.1.32), C1-6572: I.A + (H.3.4) + (K.1.32), C1-6573: I.A +
(H.4.9) + (K.1.32), C1-6574: I.A + (H.4.10) + (K.1.32), C1-6575: I.A + (I.2.2) + (K.1.32), C1-
6576: I.A + (I.2.5) + (K.1.32), C1-6577: I.A + (J.1.2) + (K.1.32), C1-6578: I.A + (J.1.5) + (K.1.32),
C1-6579: I.A + (J.1.8) + (K.1.32), C1-6580: I.A + (J.1.11) + (K.1.32), C1-6581: I.A + (J.1.12) +
(K.1.32), C1-6582: I.A + (K.1.41) + (K.1.32), C1-6583: I.A + (K.1.44) + (K.1.32), C1-6584: I.A +
(K.1.45) + (K.1.32), C1-6585: I.A + (K.1.47) + (K.1.32), C1-6586: I.A + (K.1.49) + (K.1.32), C1-
6587: I.A + (A.1.4) + (K.1.42), C1-6588: I.A + (A.1.12) + (K.1.42), C1-6589: I.A + (A.1.21) +
(K.1.42), C1-6590: I.A + (A.1,21a) + (K.1.42), C1-6591: I.A + (A.1.24) + (K.1.42), C1-6592: I.A +
(A.1.26) + (K.1.42), C1-6593: I.A + (A.1.27) + (K.1.42), C1-6594: I.A + (A.1.30) + (K.1.42), C1-
6595: I.A + (A.1.31) + (K.1.42), C1-6596: I.A + (A.1.32) + (K.1.42), C1-6597: I.A + (A.1.35) +
(K.1.42), C1-6598: I.A + (A.2.1) + (K.1.42), C1-6599: I.A + (A.2.3) + (K.1.42), C1-6600: I.A +
(A.2.5) + (K.1.42), C1-6601: I.A + (A.3.11) + (K.1.42), C1-6602: I.A + (A.3.12) + (K.1.42), C1-
6603: I.A + (A.3.15) + (K.1.42), C1-6604: I.A + (A.3.16) + (K.1.42), C1-6605: I.A + (A.3.18) +
(K.1.42), C1-6606: I.A + (A.3.19) + (K.1.42), C1-6607: I.A + (A.3.20) + (K.1.42), C1-6608: I.A +
(A.3.21) + (K.1.42), C1-6609: I.A + (A.3.22) + (K.1.42), C1-6610: I.A + (A.3.23) + (K.1.42), C1-
6611: I.A + (A.3.25) + (K.1.42), C1-6612: I.A + (A.3.26) + (K.1.42), C1-6613: I.A + (A.3.27) +
(K.1.42), C1-6614: I.A + (A.4.5) + (K.1.42), C1-6615: I.A + (A.4.11) + (K.1.42), C1-6616: I.A +
(B.1.10) + (K.1.42), C1-6617: I.A + (B.1.11) + (K.1.42), C1-6618: I.A + (B.1.12) + (K.1.42), C1-
6619: I.A + (B.1.13) + (K.1.42), C1-6620: I.A + (B.1.18) + (K.1.42), C1-6621: I.A + (B.1.21) +
(K.1.42), C1-6622: I.A + (B.1.26) + (K.1.42), C1-6623: I.A + (B.1.33) + (K.1.42), C1-6624: I.A +
(B.1.34) + (K.1.42), C1-6625: I.A + (B.1.37) + (K.1.42), C1-6626: I.A + (B.1.43) + (K.1.42), C1-
6627: I.A + (B.1.46) + (K.1.42), C1-6628: I.A + (B.2.5) + (K.1.42), C1-6629: I.A + (B.2.8) +
(K.1.42), C1-6630: I.A + (C.1.1) + (K.1.42), C1-6631: I.A + (C.1.2) + (K.1.42), C1-6632: I.A +
(C.1.4) + (K.1.42), C1-6633: I.A + (C.1.5) + (K.1.42), C1-6634: I.A + (C.2.6) + (K.1.42), C1-
6635: I.A + (C.2.7) + (K.1.42), C1-6636: I.A + (C.2.8) + (K.1.42), C1-6637: I.A + (D.1.1) +
(K.1.42), C1-6638: I.A + (D.1.2) + (K.1.42), C1-6639: I.A + (D.1.5) + (K.1.42), C1-6640: I.A +
(D.2.4) + (K.1.42), C1-6641: I.A + (E.1.3) + (K.1.42), C1-6642: I.A + (E.2.2) + (K.1.42), C1-6643:
I.A + (E.2.3) + (K.1.42), C1-6644: I.A + (F.1.2) + (K.1.42), C1-6645: I.A + (F.1.4) + (K.1.42), C1-
6646: I.A + (F.1.5) + (K.1.42), C1-6647: I.A + (G.3.1) + (K.1.42), C1-6648: I.A + (G.3.3) +
(K.1.42), C1-6649: I.A + (G.3.6) + (K.1.42), C1-6650: I.A + (G.5.1) + (K.1.42), C1-6651: I.A +
(G.5.2) + (K.1.42), C1-6652: I.A + (G.5.3) + (K.1.42), C1-6653: I.A + (G.5.4) + (K.1.42), C1-
6654: I.A + (G.5.5) + (K.1.42), C1-6655: I.A + (G.5.6) + (K.1.42), C1-6656: I.A + (G.5.7) +
(K.1.42), C1-6657: I.A + (G.5.8) + (K.1.42), C1-6658: I.A + (G.5.9) + (K.1.42), C1-6659: I.A +
(G.5.10) + (K.1.42), C1-6660: I.A + (G.5.11) + (K.1.42), C1-6661: I.A + (H.2.3) + (K.1.42), C1-
6662: I.A + (H.2.5) + (K.1.42), C1-6663: I.A + (H.2.7) + (K.1.42), C1-6664: I.A + (H.2.8) +
(K.1.42), C1-6665: I.A + (H.3.4) + (K.1.42), C1-6666: I.A + (H.4.9) + (K.1.42), C1-6667: I.A +
(H.4.10) + (K.1.42), C1-6668: I.A + (I.2.2) + (K.1.42), C1-6669: I.A + (I.2.5) + (K.1.42), C1-6670:
I.A + (J.1.2) + (K.1.42), C1-6671: I.A + (J.1.5) + (K.1.42), C1-6672: I.A + (J.1.8) + (K.1.42), C1-
6673: I.A + (J.1.11) + (K.1.42), C1-6674: I.A + (J.1.12) + (K.1.42), C1-6675: I.A + (K.1.41) +
(K.1.42), C1-6676: I.A + (K.1.44) + (K.1.42), C1-6677: I.A + (K.1.45) + (K.1.42), C1-6678: I.A +
(K.1.47) + (K.1.42), C1-6679: I.A + (K.1.49) + (K.1.42), C1-6680: I.A + (A.1.4) + (K.1.53), C1-
6681: I.A + (A.1.12) + (K.1.53), C1-6682: I.A + (A.1.21) + (K.1.53), C1-6683: I.A + (A.1,21a) +
(K.1.53), C1-6684: I.A + (A.1.24) + (K.1.53), C1-6685: I.A + (A.1.26) + (K.1.53), C1-6686: I.A +
(A.1.27) + (K.1.53), C1-6687: I.A + (A.1.30) + (K.1.53), C1-6688: I.A + (A.1.31) + (K.1.53), C1-
6689: I.A + (A.1.32) + (K.1.53), C1-6690: I.A + (A.1.35) + (K.1.53), C1-6691: I.A + (A.2.1) +
(K.1.53), C1-6692: I.A + (A.2.3) + (K.1.53), C1-6693: I.A + (A.2.5) + (K.1.53), C1-6694: I.A +
(A.3.11) + (K.1.53), C1-6695: I.A + (A.3.12) + (K.1.53), C1-6696: I.A + (A.3.15) + (K.1.53), C1-
6697: I.A + (A.3.16) + (K.1.53), C1-6698: I.A + (A.3.18) + (K.1.53), C1-6699: I.A + (A.3.19) +
(K.1.53), C1-6700: I.A + (A.3.20) + (K.1.53), C1-6701: I.A + (A.3.21) + (K.1.53), C1-6702: I.A +
(A.3.22) + (K.1.53), C1-6703: I.A + (A.3.23) + (K.1.53), C1-6704: I.A + (A.3.25) + (K.1.53), C1-
6705: I.A + (A.3.26) + (K.1.53), C1-6706: I.A + (A.3.27) + (K.1.53), C1-6707: I.A + (A.4.5) +
(K.1.53), C1-6708: I.A + (A.4.11) + (K.1.53), C1-6709: I.A + (B.1.10) + (K.1.53), C1-6710: I.A +
(B.1.11) + (K.1.53), C1-6711: I.A + (B.1.12) + (K.1.53), C1-6712: I.A + (B.1.13) + (K.1.53), C1-
6713: I.A + (B.1.18) + (K.1.53), C1-6714: I.A + (B.1.21) + (K.1.53), C1-6715: I.A + (B.1.26) +
(K.1.53), C1-6716: I.A + (B.1.33) + (K.1.53), C1-6717: I.A + (B.1.34) + (K.1.53), C1-6718: I.A +
(B.1.37) + (K.1.53), C1-6719: I.A + (B.1.43) + (K.1.53), C1-6720: I.A + (B.1.46) + (K.1.53), C1-
6721: I.A + (B.2.5) + (K.1.53), C1-6722: I.A + (B.2.8) + (K.1.53), C1-6723: I.A + (C.1.1) +
(K.1.53), C1-6724: I.A + (C.1.2) + (K.1.53), C1-6725: I.A + (C.1.4) + (K.1.53), C1-6726: I.A +
(C.1.5) + (K.1.53), C1-6727: I.A + (C.2.6) + (K.1.53), C1-6728: I.A + (C.2.7) + (K.1.53), C1-
6729: I.A + (C.2.8) + (K.1.53), C1-6730: I.A + (D.1.1) + (K.1.53), C1-6731: I.A + (D.1.2) +
(K.1.53), C1-6732: I.A + (D.1.5) + (K.1.53), C1-6733: I.A + (D.2.4) + (K.1.53), C1-6734: I.A +
(E.1.3) + (K.1.53), C1-6735: I.A + (E.2.2) + (K.1.53), C1-6736: I.A + (E.2.3) + (K.1.53), C1-6737:
I.A + (F.1.2) + (K.1.53), C1-6738: I.A + (F.1.4) + (K.1.53), C1-6739: I.A + (F.1.5) + (K.1.53), C1-
6740: I.A + (G.3.1) + (K.1.53), C1-6741: I.A + (G.3.3) + (K.1.53), C1-6742: I.A + (G.3.6) +
(K.1.53), C1-6743: I.A + (G.5.1) + (K.1.53), C1-6744: I.A + (G.5.2) + (K.1.53), C1-6745: I.A +
(G.5.3) + (K.1.53), C1-6746: I.A + (G.5.4) + (K.1.53), C1-6747: I.A + (G.5.5) + (K.1.53), C1-
6748: I.A + (G.5.6) + (K.1.53), C1-6749: I.A + (G.5.7) + (K.1.53), C1-6750: I.A + (G.5.8) +
(K.1.53), C1-6751: I.A + (G.5.9) + (K.1.53), C1-6752: I.A + (G.5.10) + (K.1.53), C1-6753: I.A +
(G.5.11) + (K.1.53), C1-6754: I.A + (H.2.3) + (K.1.53), C1-6755: I.A + (H.2.5) + (K.1.53), C1-
6756: I.A + (H.2.7) + (K.1.53), C1-6757: I.A + (H.2.8) + (K.1.53), C1-6758: I.A + (H.3.4) +
(K.1.53), C1-6759: I.A + (H.4.9) + (K.1.53), C1-6760: I.A + (H.4.10) + (K.1.53), C1-6761: I.A +
(I.2.2) + (K.1.53), C1-6762: I.A + (I.2.5) + (K.1.53), C1-6763: I.A + (J.1.2) + (K.1.53), C1-6764:
I.A + (J.1.5) + (K.1.53), C1-6765: I.A + (J.1.8) + (K.1.53), C1-6766: I.A + (J.1.11) + (K.1.53), C1-
6767: I.A + (J.1.12) + (K.1.53), C1-6768: I.A + (K.1.41) + (K.1.53), C1-6769: I.A + (K.1.44) +
(K.1.53), C1-6770: I.A + (K.1.45) + (K.1.53), C1-6771: I.A + (K.1.47) + (K.1.53), C1-6772: I.A +
(K.1.49) + (K.1.53), C1-6773: I.A + (A.1.4) + (K.1.54), C1-6774: I.A + (A.1.12) + (K.1.54), C1-
6775: I.A + (A.1.21) + (K.1.54), C1-6776: I.A + (A.1.21a) + (K.1.54), C1-6777: I.A + (A.1.24) +
(K.1.54), C1-6778: I.A + (A.1.26) + (K.1.54), C1-6779: I.A + (A.1.27) + (K.1.54), C1-6780: I.A +
(A.1.30) + (K.1.54), C1-6781: I.A + (A.1.31) + (K.1.54), C1-6782: I.A + (A.1.32) + (K.1.54), C1-
6783: I.A + (A.1.35) + (K.1.54), C1-6784: I.A + (A.2.1) + (K.1.54), C1-6785: I.A + (A.2.3) +
(K.1.54), C1-6786: I.A + (A.2.5) + (K.1.54), C1-6787: I.A + (A.3.11) + (K.1.54), C1-6788: I.A +
(A.3.12) + (K.1.54), C1-6789: I.A + (A.3.15) + (K.1.54), C1-6790: I.A + (A.3.16) + (K.1.54), C1-
6791: I.A + (A.3.18) + (K.1.54), C1-6792: I.A + (A.3.19) + (K.1.54), C1-6793: I.A + (A.3.20) +
(K.1.54), C1-6794: I.A + (A.3.21) + (K.1.54), C1-6795: I.A + (A.3.22) + (K.1.54), C1-6796: I.A +
(A.3.23) + (K.1.54), C1-6797: I.A + (A.3.25) + (K.1.54), C1-6798: I.A + (A.3.26) + (K.1.54), C1-
6799: I.A + (A.3.27) + (K.1.54), C1-6800: I.A + (A.4.5) + (K.1.54), C1-6801: I.A + (A.4.11) +
(K.1.54), C1-6802: I.A + (B.1.10) + (K.1.54), C1-6803: I.A + (B.1.11) + (K.1.54), C1-6804: I.A +
(B.1.12) + (K.1.54), C1-6805: I.A + (B.1.13) + (K.1.54), C1-6806: I.A + (B.1.18) + (K.1.54), C1-
6807: I.A + (B.1.21) + (K.1.54), C1-6808: I.A + (B.1.26) + (K.1.54), C1-6809: I.A + (B.1.33) +
(K.1.54), C1-6810: I.A + (B.1.34) + (K.1.54), C1-6811: I.A + (B.1.37) + (K.1.54), C1-6812: I.A +
(B.1.43) + (K.1.54), C1-6813: I.A + (B.1.46) + (K.1.54), C1-6814: I.A + (B.2.5) + (K.1.54), C1-
6815: I.A + (B.2.8) + (K.1.54), C1-6816: I.A + (C.1.1) + (K.1.54), C1-6817: I.A + (C.1.2) +
(K.1.54), C1-6818: I.A + (C.1.4) + (K.1.54), C1-6819: I.A + (C.1.5) + (K.1.54), C1-6820: I.A +
(C.2.6) + (K.1.54), C1-6821: I.A + (C.2.7) + (K.1.54), C1-6822: I.A + (C.2.8) + (K.1.54), C1-
6823: I.A + (D.1.1) + (K.1.54), C1-6824: I.A + (D.1.2) + (K.1.54), C1-6825: I.A + (D.1.5) +
(K.1.54), C1-6826: I.A + (D.2.4) + (K.1.54), C1-6827: I.A + (E.1.3) + (K.1.54), C1-6828: I.A +
(E.2.2) + (K.1.54), C1-6829: I.A + (E.2.3) + (K.1.54), C1-6830: I.A + (F.1.2) + (K.1.54), C1-6831:
I.A + (F.1.4) + (K.1.54), C1-6832: I.A + (F.1.5) + (K.1.54), C1-6833: I.A + (G.3.1) + (K.1.54), C1-
6834: I.A + (G.3.3) + (K.1.54), C1-6835: I.A + (G.3.6) + (K.1.54), C1-6836: I.A + (G.5.1) +
(K.1.54), C1-6837: I.A + (G.5.2) + (K.1.54), C1-6838: I.A + (G.5.3) + (K.1.54), C1-6839: I.A +
(G.5.4) + (K.1.54), C1-6840: I.A + (G.5.5) + (K.1.54), C1-6841: I.A + (G.5.6) + (K.1.54), C1-
6842: I.A + (G.5.7) + (K.1.54), C1-6843: I.A + (G.5.8) + (K.1.54), C1-6844: I.A + (G.5.9) +
(K.1.54), C1-6845: I.A + (G.5.10) + (K.1.54), C1-6846: I.A + (G.5.11) + (K.1.54), C1-6847: I.A +
(H.2.3) + (K.1.54), C1-6848: I.A + (H.2.5) + (K.1.54), C1-6849: I.A + (H.2.7) + (K.1.54), C1-
6850: I.A + (H.2.8) + (K.1.54), C1-6851: I.A + (H.3.4) + (K.1.54), C1-6852: I.A + (H.4.9) +
(K.1.54), C1-6853: I.A + (H.4.10) + (K.1.54), C1-6854: I.A + (I.2.2) + (K.1.54), C1-6855: I.A +
(I.2.5) + (K.1.54), C1-6856: I.A + (J.1.2) + (K.1.54), C1-6857: I.A + (J.1.5) + (K.1.54), C1-6858:
I.A + (J.1.8) + (K.1.54), C1-6859: I.A + (J.1.11) + (K.1.54), C1-6860: I.A + (J.1.12) + (K.1.54),
C1-6861: I.A + (K.1.41) + (K.1.54), C1-6862: I.A + (K.1.44) + (K.1.54), C1-6863: I.A + (K.1.45) +
(K.1.54), C1-6864: I.A + (K.1.47) + (K.1.54), C1-6865: I.A + (K.1.49) + (K.1.54).

Table C2: Mixtures C2-1 to C2-6865 are defined as mixtures C1-1 to C1-6865 of Table C1, wherein compound I.A in each mixture is replaced with compound I.B.

Table C3: Mixtures C3-1 to C3-6865 are defined as mixtures C1-1 to C1-6865 of Table C1, wherein compound I.A in each mixture is replaced with compound I.C.

Table C4: Mixtures C4-1 to C4-6865 are defined as mixtures C1-1 to C1-6865 of Table C1, wherein compound I.A in each mixture is replaced with compound I.D.

Table C5: Mixtures C5-1 to C5-6865 are defined as mixtures C1-1 to C1-6865 of Table C1, wherein compound I.A in each mixture is replaced with compound I.E.

Table C6: Mixtures C6-1 to C6-6865 are defined as mixtures C1-1 to C1-6865 of Table C1, wherein compound I.A in each mixture is replaced with compound I.F.

In one embodiment the weight ratio of compound I to compound II is between 100:1 and 1:100; and the weight ratio of compound I to compound III, is between 100:1 and 1:100 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In one embodiment the weight ratio of compound I to compound II is between 100:1 and 1:100; and the weight ratio of compound I to compound III, is between 50:1 and 1:50 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In one embodiment the weight ratio of compound I to compound II is between 100:1 and 1:100; and the weight ratio of compound I to compound III, is between 20:1 and 1:20 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In one embodiment the weight ratio of compound I to compound II is between 100:1 and 1:100; and the weight ratio of compound I to compound III, is between 5:1 and 1:5 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In one embodiment the weight ratio of compound I to compound II is between 100:1 and 1:100; and the weight ratio of compound I to compound III is, for example, 3:1, 1:1 or 1:3 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In one embodiment the weight ratio of compound I to compound II is between 50:1 and 1:50; and the weight ratio of compound I to compound III, is between 100:1 and 1:100 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In one embodiment the weight ratio of compound I to compound II is between 50:1 and 1:50; and the weight ratio of compound I to compound III, is between 50:1 and 1:50 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In one embodiment the weight ratio of compound I to compound II is between 50:1 and 1:50; and the weight ratio of compound I to compound III, is between 20:1 and 1:20 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In one embodiment the weight ratio of compound I to compound II is between 50:1 and 1:50; and the weight ratio of compound I to compound III, is between 5:1 and 1:5 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In one embodiment the weight ratio of compound I to compound II is between 50:1 and 1:50; and the weight ratio of compound I to compound III is, for example, 3:1, 1:1 or 1:3 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In one embodiment the weight ratio of compound I to compound II is between 20:1 and 1:20; and the weight ratio of compound I to compound III, is between 100:1 and 1:100 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In one embodiment the weight ratio of compound I to compound II is between 20:1 and 1:20; and the weight ratio of compound I to compound III, is between 50:1 and 1:50 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In one embodiment the weight ratio of compound I to compound II is between 20:1 and 1:20; and the weight ratio of compound I to compound III, is between 20:1 and 1:20 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In one embodiment the weight ratio of compound I to compound II is between 20:1 and 1:20; and the weight ratio of compound I to compound III, is between 5:1 and 1:5 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In one embodiment the weight ratio of compound I to compound II is between 20:1 and 1:20; and the weight ratio of compound I to compound III is, for example, 3:1, 1:1 or 1:3 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In one embodiment the weight ratio of compound I to compound II is between 5:1 and 1:5; and the weight ratio of compound I to compound III, is between 50:1 and 1:50 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In one embodiment the weight ratio of compound I to compound II is between 5:1 and 1:5; and the weight ratio of compound I to compound III, is between 20:1 and 1:20 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In one embodiment the weight ratio of compound I to compound II is between 5:1 and 1:5; and the weight ratio of compound I to compound III, is between 5:1 and 1:5 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In embodiment to one embodiment the weight ratio of compound I to compound II is between 5:1 and 1:5; and the weight ratio of compound I to compound III is, for example, 3:1, 1:1 or 1:3 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In embodiment the weight ratio of compound I to compound II is 3:1; and the weight ratio of compound I to compound III is 3:1 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In embodiment the weight ratio of compound I to compound II is 3:1; and the weight ratio of compound I to compound III is 1:1 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In embodiment the weight ratio of compound I to compound II is 3:1; and the weight ratio of compound I to compound III is 1:3 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In embodiment the weight ratio of compound I to compound II is 1:1; and the weight ratio of compound I to compound III is 3:1 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

According one embodiment the weight ratio of compound I to compound II is 1:1; and the weight ratio of compound I to compound III is 1:1 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In embodiment the weight ratio of compound I to compound II is 1:1; and the weight ratio of compound I to compound III is 1:3 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In embodiment the weight ratio of compound I to compound II is 1:3; and the weight ratio of compound I to compound III is 3:1 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In embodiment the weight ratio of compound I to compound II is 1:3; and the weight ratio of compound I to compound III is 1:1 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In embodiment the weight ratio of compound I to compound II is 1:3; and the weight ratio of compound I to compound III is 1:3 in mixtures C1-1 to C1-6865, C2-1 to C2-6865, C3-1 to C3-6865, C4-1 to C4-6865, C5-1 to C5-6865, and C6-1 to C6-6865 of Tables C1 to C6.

In a preferred embodiment the mixtures C1-1 to C1-6865, C2-1 to C2-6865, C3-1 to C3-6865, C4-1 to C4-6865, C5-1 to C5-6865, and C6-1 to C6-6865 of Tables C1 to C6 and in particular when the weight ratios of compound I to compound and of compound I and compound III are as disclosed for these mixtures as herein before mentioned, are used against Phakopsora pachyrhizi and P. meibomiae on soybeans and/or against Puccinia triticina, P. striiformis, P. hordei, P. graminis or P. recondita on wheat, barley or rye.

Other preferred ternary mixtures, preferably for use against Phakopsora pachyrhizi and P. meibomiae on soybeans and/or against Puccinia triticina, P. striiformis, P. hordei, P. graminis or P. recondita on wheat, barley or rye, are listed in Table D below comprising as component 1) one of the compounds I.A, I.B, I.C, I.D, I.E, I.F, I.G, I.H, I.J, I.K, I.L, I.M or I.N as defined above, and as component 2) one of the compounds II as defined and numbered above, and as component 3) a compound III from groups A) to K) as defined herein. Preferably, the compositions comprise the active substances in synergistically effective amounts. In the ternary mixtures D-1 to D-156 the components 1), 2) and 3) are written down in this order (comp. 1)+comp. 2)+comp. 3)), so that one mixture, for example mixture D1-1, comprises as component 1) compound I.A, as component 2) compound (A.1.1) and as component 3) compound (A.3.2).

TABLE D
D-1: I.A + (B.1.38) + (B.2.4), D-2: I.A + (B.1.38) + (A.1.25), D-3: I.A + (B.1.38) + (A.1.14), D-4: I.A +
(B.1.38) + (A.3.9), D-5: I.A + (B.1.38) + (B.1.23), D-6: I.A + (B.1.38) + (A.1.1), D-7: I.A + (B.1.38) +
(A.1.34), D-8: I.A + (B.1.38) + (D.2.6), D-9: I.A + (A.3.9) + (A.1.14), D-10: I.A + (A.3.9) + (A.1.1), D-11:
I.A + (A.3.9) + (A.1.25), D-12: I.A + (A.3.9) + (A.1.34), D-13: I.A + (A.3.9) + (D.2.6), D-14: I.A + (A.3.9) +
(B.2.4), D-15: I.A + (A.3.9) + (H.2.2), D-16: I.A + (A.3.9) + (H.3.2), D-17: I.A + (A.3.9) + (H.1.5), D-18:
I.A + (A.1.25) + (A.1.1), D-19: I.A + (A.1.25) + (A.1.14), D-20: I.A + (A.1.25) + (A.1.34), D-21: I.A + (A.1.25) +
(D2.6), D-22: I.A + (A.3.9) + (B.1.23), D-23: I.A + (B.1.23) + (B.2.4), D-24: I.A + (B.1.23) + (H.2.2), D-25:
I.A + (B.1.23) + (H.3.2), D-26: I.A + (B.1.23) + (H.1.5), D-27: I.B + (B.1.38) + (B.2.4), D-28: I.B +
(B.1.38) + (A.1.25), D-29: I.B + (B.1.38) + (A.1.14), D-30: I.B + (B.1.38) + (A.3.9), D-31: I.B + (B.1.38) +
(B.1.23), D-32: I.B + (B.1.38) + (A.1.1), D-33: I.B + (B.1.38) + (A.1.34), D-34: I.B + (B.1.38) + (D.2.6), D-35:
I.B + (A.3.9) + (A.1.14), D-36: I.B + (A.3.9) + (A.1.1), D-37: I.B + (A.3.9) + (A.1.25), D-38: I.B + (A.3.9) +
(A.1.34), D-39: I.B + (A.3.9) + (D.2.6), D-40: I.B + (A.3.9) + (B.2.4), D-41: I.B + (A.3.9) + (H.2.2), D-42:
I.B + (A.3.9) + (H.3.2), D-43: I.B + (A.3.9) + (H.1.5), D-44: I.B + (A.1.25) + (A.1.1), D-45: I.B + (A.1.25) +
(A.1.14), D-46: I.B + (A.1.25) + (A.1.34), D-47: I.B + (A.1.25) + (D2.6), D-48: I.B + (A.3.9) + (B.1.23),
D-49: I.B + (B.1.23) + (B.2.4), D-50: I.B + (B.1.23) + (H.2.2), D-51: I.B + (B.1.23) + (H.3.2), D-52: I.B +
(B.1.23) + (H.1.5), D-53: I.C + (B.1.38) + (B.2.4), D-54: I.C + (B.1.38) + (A.1.25), D-55: I.C + (B.1.38) +
(A.1.14), D-56: I.C + (B.1.38) + (A.3.9), D-57: I.C + (B.1.38) + (B.1.23), D-58: I.C + (B.1.38) + (A.1.1),
D-59: I.C + (B.1.38) + (A.1.34), D-60: I.C + (B.1.38) + (D.2.6), D-61: I.C + (A.3.9) + (A.1.14), D-62: I.C +
(A.3.9) + (A.1.1), D-63: I.C + (A.3.9) + (A.1.25), D-64: I.C + (A.3.9) + (A.1.34), D-65: I.C + (A.3.9) +
(D.2.6), D-66: I.C + (A.3.9) + (B.2.4), D-67: I.C + (A.3.9) + (H.2.2), D-68: I.C + (A.3.9) + (H.3.2), D-69:
I.C + (A.3.9) + (H.1.5), D-70: I.C + (A.1.25) + (A.1.1), D-71: I.C + (A.1.25) + (A.1.14), D-72: I.C + (A.1.25) +
(A.1.34), D-73: I.C + (A.1.25) + (D2.6), D-74: I.C + (A.3.9) + (B.1.23), D-75: I.C + (B.1.23) + (B.2.4), D-76:
I.C + (B.1.23) + (H.2.2), D-77: I.C + (B.1.23) + (H.3.2), D-78: I.C + (B.1.23) + (H.1.5), D-79: I.D +
(B.1.38) + (B.2.4), D-80: I.D + (B.1.38) + (A.1.25), D-81: I.D + (B.1.38) + (A.1.14), D-82: I.D + (B.1.38) +
(A.3.9), D-83: I.D + (B.1.38) + (B.1.23), D-84: I.D + (B.1.38) + (A.1.1), D-85: I.D + (B.1.38) + (A.1.34), D-86:
I.D + (B.1.38) + (D.2.6), D-87: I.D + (A.3.9) + (A.1.14), D-88: I.D + (A.3.9) + (A.1.1), D-89: I.D + (A.3.9) +
(A.1.25), D-90: I.D + (A.3.9) + (A.1.34), D-91: I.D + (A.3.9) + (D.2.6), D-92: I.D + (A.3.9) + (B.2.4), D-93:
I.D + (A.3.9) + (H.2.2), D-94: I.D + (A.3.9) + (H.3.2), D-95: I.D + (A.3.9) + (H.1.5), D-96: I.D + (A.1.25) +
(A.1.1), D-97: I.D + (A.1.25) + (A.1.14), D-98: I.D + (A.1.25) + (A.1.34), D-99: I.D + (A.1.25) + (D2.6),
D-100: I.D + (A.3.9) + (B.1.23), D-101: I.D + (B.1.23) + (B.2.4), D-102: I.D + (B.1.23) + (H.2.2), D-103: I.D +
(B.1.23) + (H.3.2), D-104: I.D + (B.1.23) + (H.1.5), D-105: I.E + (B.1.38) + (B.2.4), D-106: I.E + (B.1.38) +
(A.1.25), D-107: I.E + (B.1.38) + (A.1.14), D-108: I.E + (B.1.38) + (A.3.9), D-109: I.E + (B.1.38) + (B.1.23),
D-110: I.E + (B.1.38) + (A.1.1), D-111: I.E + (B.1.38) + (A.1.34), D-112: I.E + (B.1.38) + (D.2.6), D-113:
I.E + (A.3.9) + (A.1.14), D-114: I.E + (A.3.9) + (A.1.1), D-115: I.E + (A.3.9) + (A.1.25), D-116: I.E +
(A.3.9) + (A.1.34), D-117: I.E + (A.3.9) + (D.2.6), D-118: I.E + (A.3.9) + (B.2.4), D-119: I.E + (A.3.9) + (H.2.2),
D-120: I.E + (A.3.9) + (H.3.2), D-121: I.E + (A.3.9) + (H.1.5), D-122: I.E + (A.1.25) + (A.1.1), D-123: I.E +
(A.1.25) + (A.1.14), D-124: I.E + (A.1.25) + (A.1.34), D-125: I.E + (A.1.25) + (D2.6), D-126: I.E + (A.3.9) +
(B.1.23), D-127: I.E + (B.1.23) + (B.2.4), D-128: I.E + (B.1.23) + (H.2.2), D-129: I.E + (B.1.23) + (H.3.2),
D-130: I.E + (B.1.23) + (H.1.5), D-131: I.F + (B.1.38) + (B.2.4), D-132: I.F + (B.1.38) + (A.1.25), D-133:
I.F + (B.1.38) + (A.1.14), D-134: I.F + (B.1.38) + (A.3.9), D-135: I.F + (B.1.38) + (B.1.23), D-136: I.F +
(B.1.38) + (A.1.1), D-137: I.F + (B.1.38) + (A.1.34), D-138: I.F + (B.1.38) + (D.2.6), D-139: I.F + (A.3.9) +
(A.1.14), D-140: I.F + (A.3.9) + (A.1.1), D-141: I.F + (A.3.9) + (A.1.25), D-142: I.F + (A.3.9) + (A.1.34),
D-143: I.F + (A.3.9) + (D.2.6), D-144: I.F + (A.3.9) + (B.2.4), D-145: I.F + (A.3.9) + (H.2.2), D-146: I.F +
(A.3.9) + (H.3.2), D-147: I.F + (A.3.9) + (H.1.5), D-148: I.F + (A.1.25) + (A.1.1), D-149: I.F + (A.1.25) +
(A.1.14), D-150: I.F + (A.1.25) + (A.1.34), D-151: I.F + (A.1.25) + (D2.6), D-152: I.F + (A.3.9) + (B.1.23),
D-153: I.F + (B.1.23) + (B.2.4), D-154: I.F + (B.1.23) + (H.2.2), D-155: I.F + (B.1.23) + (H.3.2), D-156:
I.F + (B.1.23) + (H.1.5).

In one embodiment the weight ratio of compound I to compound II is between 100:1 and 1:100; and the weight ratio of compound I to compound III, is between 100:1 and 1:100 in mixtures D-1 to D-156 of Table D.

In one embodiment the weight ratio of compound I to compound II is between 100:1 and 1:100; and the weight ratio of compound I to compound III, is between 50:1 and 1:50 in mixtures D-1 to D-156 of Table D.

In one embodiment the weight ratio of compound I to compound II is between 100:1 and 1:100; and the weight ratio of compound I to compound III, is between 20:1 and 1:20 in mixtures D-1 to D-156 of Table D.

In one embodiment the weight ratio of compound I to compound II is between 100:1 and 1:100; and the weight ratio of compound I to compound III, is between 5:1 and 1:5 in mixtures D-1 to D-156 of Table D.

In one embodiment the weight ratio of compound I to compound II is between 100:1 and 1:100; and the weight ratio of compound I to compound III is, for example, 3:1, 1:1 or 1:3 in mixtures D-1 to D-156 of Table D.

In one embodiment the weight ratio of compound I to compound II is between 50:1 and 1:50; and the weight ratio of compound I to compound III, is between 100:1 and 1:100 in mixtures D-1 to D-156 of Table D.

In one embodiment the weight ratio of compound I to compound II is between 50:1 and 1:50; and the weight ratio of compound I to compound III, is between 50:1 and 1:50 in mixtures D-1 to D-156 of Table D.

In one embodiment the weight ratio of compound I to compound II is between 50:1 and 1:50; and the weight ratio of compound I to compound III, is between 20:1 and 1:20 in mixtures D-1 to D-156 of Table D.

In one embodiment the weight ratio of compound I to compound II is between 50:1 and 1:50; and the weight ratio of compound I to compound III, is between 5:1 and 1:5 in mixtures D-1 to D-156 of Table D.

In one embodiment the weight ratio of compound I to compound II is between 50:1 and 1:50; and the weight ratio of compound I to compound III is 3:1 in mixtures D-1 to D-156 of Table D.

In one embodiment the weight ratio of compound I to compound II is between 100:1 and 1:100; and the weight ratio of compound I to compound III is 1:1 in mixtures D-1 to D-156 of Table D.

In one embodiment the weight ratio of compound I to compound II is between 100:1 and 1:100; and the weight ratio of compound I to compound III is 1:3 in mixtures D-1 to D-156 of Table D.

In one embodiment the weight ratio of compound I to compound II is between 50:1 and 1:50; and the weight ratio of compound I to compound III, is between 100:1 and 1:100 in mixtures D-1 to D-156 of Table D.

In one embodiment the weight ratio of compound I to compound II is between 20:1 and 1:20; and the weight ratio of compound I to compound III, is between 50:1 and 1:50 in mixtures D-1 to D-156 of Table D.

In one embodiment the weight ratio of compound I to compound II is between 20:1 and 1:20; and the weight ratio of compound I to compound III, is between 20:1 and 1:20 in mixtures D-1 to D-156 of Table D.

In one embodiment the weight ratio of compound I to compound II is between 20:1 and 1:20; and the weight ratio of compound I to compound III, is between 5:1 and 1:5 in mixtures D-1 to D-156 of Table D.

In one embodiment the weight ratio of compound I to compound II is between 20:1 and 1:20; and the weight ratio of compound I to compound III is 3:1 in mixtures D-1 to D-156 of Table D.

In one embodiment the weight ratio of compound I to compound II is between 50:1 and 1:50; and the weight ratio of compound I to compound III is 1:1 in mixtures D-1 to D-156 of Table D.

In one embodiment the weight ratio of compound I to compound II is between 50:1 and 1:50; and the weight ratio of compound I to compound III is 1:3 in mixtures D-1 to D-156 of Table D.

In one embodiment the weight ratio of compound I to compound II is between 5:1 and 1:5; and the weight ratio of compound I to compound III, is between 50:1 and 1:50 in mixtures D-1 to D-156 of Table D.

In one embodiment the weight ratio of compound I to compound II is between 5:1 and 1:5; and the weight ratio of compound I to compound III, is between 20:1 and 1:20 in mixtures D-1 to D-156 of Table D.

In one embodiment the weight ratio of compound I to compound II is between 5:1 and 1:5; and the weight ratio of compound I to compound III, is between 5:1 and 1:5 in mixtures D-1 to D-156 of Table D.

In one embodiment the weight ratio of compound I to compound II is between 20:1 and 1:20; and the weight ratio of compound I to compound III is 3:1 in mixtures D-1 to D-156 of Table D.

In one embodiment the weight ratio of compound I to compound II is between 20:1 and 1:20; and the weight ratio of compound I to compound III is 1:1 in mixtures D-1 to D-156 of Table D.

In one embodiment the weight ratio of compound I to compound II is between 20:1 and 1:20; and the weight ratio of compound I to compound III is 1:3 in mixtures D-1 to D-156 of Table D.

In one embodiment the weight ratio of compound I to compound II is between 5:1 and 1:5; and the weight ratio of compound I to compound III is 3:1 in mixtures D-1 to D-156 of Table D.

In one embodiment the weight ratio of compound I to compound II is between 5:1 and 1:5; and the weight ratio of compound I to compound III is 1:1 in mixtures D-1 to D-156 of Table D.

In one embodiment the weight ratio of compound I to compound II is between 5:1 and 1:5; and the weight ratio of compound I to compound III is 1:3 in mixtures D-1 to D-156 of Table D.

In one embodiment the weight ratio of compound I to compound II is 3:1; and the weight ratio of compound I to compound III is 3:1 in mixtures D-1 to D-156 of Table D.

In one embodiment the weight ratio of compound I to compound II is 3:1; and the weight ratio of compound I to compound III is 1:1 in mixtures D-1 to D-156 of Table D.

In one embodiment the weight ratio of compound I to compound II is 3:1; and the weight ratio of compound I to compound III is 1:3 in mixtures D-1 to D-156 of Table D.

In one embodiment the weight ratio of compound I to compound II is 1:1; and the weight ratio of compound I to compound III is 3:1 in mixtures D-1 to D-156 of Table D.

In one embodiment the weight ratio of compound I to compound II is 1:1; and the weight ratio of compound I to compound III is 1:1 in mixtures D-1 to D-156 of Table D.

In one embodiment the weight ratio of compound I to compound II is 1:1; and the weight ratio of compound I to compound III is 1:3 in mixtures D-1 to D-156 of Table D.

In a preferred embodiment the mixtures D-1 to D-156 of Table D and in particular when the weight ratios of compound I to compound II and of compound I and compound III are as disclosed for these mixtures as herein before mentioned, are used against Phakopsora pachyrhizi and P. meibomiae on soybeans and/or against Puccinia triticina, P. striiformis, P. hordei P. graminis or P. recondita on wheat, barley or rye.

The binary and ternary mixtures and agrochemical compositions thereof according to the invention can, in the use form as fungicides, also be present together with further active substances, e. g. with herbicides, insecticides, growth regulators, fungicides or else with fertilizers, as pre-mix or, if appropriate, not until immediately prior to use (tank mix). Mixing the active compounds I, and III and the compositions comprising the mixtures, respectively, in the use form as fungicides with other fungicides results in many cases in an expansion of the fungicidal spectrum of activity being obtained or in a prevention of fungicide resistance development. Furthermore, in many cases, synergistic effects are obtained.

The compounds of the formula I or compositions comprising said compounds according to the invention and the mixtures comprising said compounds and compositions, respectively, are suitable as fungicides. They are distinguished by an outstanding effectiveness against a broad spectrum of phytopathogenic fungi, including soil-borne fungi, which derive especially from the following classes or are closely related to any of them: Ascomycota (Ascomycetes), for example, but not limited to the genus Cocholiobolus, Colletotrichum, Fusarium, Microdochium, Penicillium, Phoma, Magnaporte, Zymoseptoria, and Pseudocercosporella; Basdiomycota (Basidiomycetes), for example, but not limited to the genus Phakospora, Puccinia, Rhizoctonia, Sphacelotheca, Tilletia, Typhula, and Ustilago; Chytridiomycota (Chytridiomycetes), for example, but not limited to the genus Chytridiales, and Synchytrium; Deuteromycetes (syn. Fungi imperfecti), for example, but not limited to the genus Ascochyta, Dlodia, Erysiphe, Fusarium, Phomopsis, and Pyrenophora; Peronosporomycetes (syn. Oomycetes), for example but not limited to the genus Peronospora, Pythium, Phytophthora; Plasmodiophoromycetes, for example but not limited to the genus Plasmodiophora; Zygomycetes, for example, but not limited to the genus Rhizopus.

Some of the compounds of the formula I and the compositions according to the invention are systemically effective and they can be used in crop protection as foliar fungicides, fungicides for seed dressing and soil fungicides. Moreover, they are suitable for controlling harmful fungi, which inter alia occur in wood or roots of plants.

The compounds I and the compositions according to the invention are particularly important in the control of a multitude of phytopathogenic fungi on various cultivated plants, such as cereals, e. g. wheat, rye, barley, triticale, oats or rice; beet, e. g. sugar beet or fodder beet; fruits, such as pomes, stone fruits or soft fruits, e. g. apples, pears, plums, peaches, almonds, cherries, strawberries, raspberries, blackberries or gooseberries; leguminous plants, such as lentils, peas, alfalfa or soybeans; oil plants, such as rape, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, sugar cane or oil palm; corn; tobacco; nuts; coffee; tea; bananas; vines (table grapes and grape juice grape vines); hop; turf; sweet leaf (also called Stevia); natural rubber plants or ornamental and forestry plants, such as flowers, shrubs, broad-leaved trees or evergreens, e. g. conifers; and on the plant propagation material, such as seeds, and the crop material of these plants. Preferably, compounds I and compositions thereof, respectively are used for controlling a multitude of fungi on field crops, such as potatoes sugar beets, tobacco, wheat, rye, barley, oats, rice, corn, cotton, soybeans, rape, legumes, sunflowers, coffee or sugar cane; fruits; vines; ornamentals; or vegetables, such as cucumbers, tomatoes, beans or squashes.

The term “plant propagation material” is to be understood to denote all the generative parts of the plant such as seeds and vegetative plant material such as cuttings and tubers (e. g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil.

These young plants may also be protected before transplantation by a total or partial treatment by immersion or pouring.

Preferably, treatment of plant propagation materials with compounds I and compositions thereof, respectively, is used for controlling a multitude of fungi on cereals, such as wheat, rye, barley and oats; rice, corn, cotton and soybeans.

The term “cultivated plants” is to be understood as including plants which have been modified by breeding, mutagenesis or genetic engineering including but not limiting to agricultural biotech products on the market or in development (cf. http://cera-gmc.org/, see GM crop database therein).

The compounds I, and mixtures comprising them, and agrochemical compositions thereof, respectively, are particularly suitable for controlling the following plant diseases: Albugo spp. (white rust) on ornamentals, vegetables (e. g. A. candida) and sunflowers (e. g. A. tragopogonis); Alternaria spp. (Alternaria leaf spot) on vegetables, rape (A. brassicola or brassicae), sugar beets (A. tenuis), fruits, rice, soybeans, potatoes (e. g. A. solani or A. alternata), tomatoes (e. g. A. solani or A. alternata) and wheat; Aphanomyces spp. on sugar beets and vegetables; Ascochyta spp. on cereals and vegetables, e. g. A. tritici (anthracnose) on wheat and A. hordei on barley; Bipolaris and Drechslera spp. (teleomorph: Cochliobolus spp.), e. g. Southern leaf blight (D. maydis) or Northern leaf blight (B. zeicola) on corn, e. g. spot blotch (B. sorokiniana) on cereals and e. g. B. oryzae on rice and turfs; Blumeria (formerly Erysiphe) graminis (powdery mildew) on cereals (e. g. on wheat or barley); Botrytis cinerea (teleomorph: Botryotinia fuckeliana: grey mold) on fruits and berries (e. g. strawberries), vegetables (e. g. lettuce, carrots, celery and cabbages), rape, flowers, vines, forestry plants and wheat; Bremia lactucae (downy mildew) on lettuce; Ceratocystis (syn. Ophiostoma) spp. (rot or wilt) on broad-leaved trees and evergreens, e. g. C. ulmi (Dutch elm disease) on elms; Cercospora spp. (Cercospora leaf spots) on corn (e. g. Gray leaf spot: C. zeae-maydis), rice, sugar beets (e. g. C. beticola), sugar cane, vegetables, coffee, soybeans (e. g. C. sojina or C. kikuchii) and rice; Cladosporium spp. on tomatoes (e. g. C. fulvum: leaf mold) and cereals, e. g. C. herbarum (black ear) on wheat; Claviceps purpurea (ergot) on cereals; Cochliobolus (anamorph: Helminthosporium of Bipolaris) spp. (leaf spots) on corn (C. carbonum), cereals (e. g. C. sativus, anamorph: B. sorokiniana) and rice (e. g. C. miyabeanus, anamorph: H. oryzae); Colletotrichum (teleomorph: Glomerella) spp. (anthracnose) on cotton (e. g. C. gossypii), corn (e. g. C. graminicola: Anthracnose stalk rot), soft fruits, potatoes (e. g. C. coccodes: black dot), beans (e. g. C. lindemuthianum) and soybeans (e. g. C. truncatum or C. gloeosporioides); Corticium spp., e. g. C. sasakii (sheath blight) on rice; Corynespora cassiicola (leaf spots) on soybeans and ornamentals; Cycloconium spp., e. g. C. oleaginum on olive trees; Cylindrocarpon spp. (e. g. fruit tree canker or young vine decline, teleomorph: Nectria or Neonectria spp.) on fruit trees, vines (e. g. C. lriodendri, teleomorph: Neonectria liriodendri: Black Foot Disease) and ornamentals; Dematophora (teleomorph: Rosellinia) necatrix (root and stem rot) on soybeans; Diaporthe spp., e. g. D. phaseolorum (damping off) on soybeans; Drechslera (syn. Helminthosporium, teleomorph: Pyrenophora) spp. on corn, cereals, such as barley (e. g. D. teres, net blotch) and wheat (e. g. D. tritici-repentis: tan spot), rice and turf; Esca (dieback, apoplexy) on vines, caused by Formitiporia (syn. Phellinus) punctata, F. mediterranea, Phaeomoniella chlamydospora (earlier Phaeoacremonium chlamydosporum), Phaeoacremonium aleophilum and/or Botryosphaeria obtusa; Elsinoe spp. on pome fruits (E. pyri), soft fruits (E. veneta: anthracnose) and vines (E. ampelina: anthracnose); Entyloma oryzae (leaf smut) on rice; Epicoccum spp. (black mold) on wheat; Erysiphe spp. (powdery mildew) on sugar beets (E. betae), vegetables (e. g. E. pisi), such as cucurbits (e. g. E. cichoracearum), cabbages, rape (e. g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e. g. E. turcicum); Fusarium (teleomorph: Gibberella) spp. (wilt, root or stem rot) on various plants, such as F. graminearum or F. culmorum (root rot, scab or head blight) on cereals (e. g. wheat or barley), F. oxysporum on tomatoes, F. solani(f. sp. glycines now syn. F. virguliforme) and F. tucumaniae and F. brasiliense each causing sudden death syndrome on soybeans, and F. verticillioides on corn; Gaeumannomyces graminis (take-all) on cereals (e. g. wheat or barley) and corn; Gibberella spp. on cereals (e. g. G. zeae) and rice (e. g. G. fujikuroi Bakanae disease); Glomerella cingulata on vines, pome fruits and other plants and G. gossypii on cotton; Grainstaining complex on rice; Gugnardia bidwellii (black rot) on vines; Gymnosporangium spp. on rosaceous plants and junipers, e. g. G. sabinae (rust) on pears; Helminthosporium spp. (syn. Drechslera, teleomorph: Cochliobolus) on corn, cereals and rice; Hemileia spp., e. g. H. vastatri (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e. g. wheat or barley); Microsphaera diffusa (powdery mildew) on soybeans; Monilinia spp., e. g. M. laxa, M. fructicola and M. fructigena (bloom and twig blight, brown rot) on stone fruits and other rosaceous plants; Mycosphaerella spp. on cereals, bananas, soft fruits and ground nuts, such as e. g. M. graminicola (anamorph: Septoria tritici, Septoria blotch) on wheat or M. fijiensis (black Sigatoka disease) on bananas; Peronospora spp. (downy mildew) on cabbage (e. g. P. brassicae), rape (e. g. P. parasitica), onions (e. g. P. destructor), tobacco (P. tabacina) and soybeans (e. g. P. manshurica); Phakopsora pachyrhizi and P. meibomiae (soybean rust) on soybeans; Phialophora spp. e. g. on vines (e. g. P. tracheiphila and P. tetraspora) and soybeans (e. g. P. gregata: stem rot); Phoma lingam (root and stem rot) on rape and cabbage and P. betae (root rot, leaf spot and damping-off) on sugar beets; Phomopsis spp. on sunflowers, vines (e. g. P. viticola: can and leaf spot) and soybeans (e. g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e. g. P. capsici), soybeans (e. g. P. megasperma, syn. P. sojae), potatoes and tomatoes (e. g. P. infestans: late blight) and broad-leaved trees (e. g. P. ramorum: sudden oak death); Plasmodiophora brassicae (club root) on cabbage, rape, radish and other plants; Plasmopara spp., e. g. P. v//cola (grapevine downy mildew) on vines and P. halstedii on sunflowers; Podosphaera spp. (powdery mildew) on rosa-ceous plants, hop, pome and soft fruits, e. g. P. leucotricha on apples; Polymyxa spp., e. g. on cereals, such as barley and wheat (P. graminis) and sugar beets (P. betae) and thereby transmitted viral diseases; Pseudocercosporella herpotrichoides (eyespot, teleomorph: Tapesia yallundae) on cereals, e. g. wheat or barley; Pseudoperonospora (downy mildew) on various plants, e. g. P. cubensis on cucurbits or P. humili on hop; Pseudopezicula tracheiphila (red fire disease or ‘rotbrenner’, anamorph: Phialophora) on vines; Puccinia spp. (rusts) on various plants, e. g. P. triticina (brown or leaf rust), P. striformis (stripe or yellow rust), P. hordei (dwarf rust), P. graminis (stem or black rust) or P. recondita (brown or leaf rust) on cereals, such as e. g. wheat, barley or rye, P. kuehnii (orange rust) on sugar cane and P. asparagion asparagus; Pyrenophora (anamorph: Drechslera) tritici-repentis (tan spot) on wheat or P. teres (net blotch) on barley; Pyricularia spp., e. g. P. oryzae (teleomorph: Magnaporthe gr/sea, rice blast) on rice and P. grisea on turf and cereals; Pythium spp. (damping-off) on turf, rice, corn, wheat, cotton, rape, sunflowers, soybeans, sugar beets, vegetables and various other plants (e. g. P. ultimum or P. aphanidermatum); Ramularia spp., e. g. R. collo-cygni(Ramularia leaf spots, Physiological leaf spots) on barley and R. beticola on sugar beets; Rhizoctonia spp. on cotton, rice, potatoes, turf, corn, rape, potatoes, sugar beets, vegetables and various other plants, e. g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis (scald) on barley, rye and triticale; Sarocladium oryzae and S. attenuatum (sheath rot) on rice; Sclerotinia spp. (stem rot or white mold) on vegetables and field crops, such as rape, sunflowers (e. g. S. sclerotiorum) and soybeans (e. g. S. rolfsii or S. sclerotiorum); Septoria spp. on various plants, e. g. S. glycines (brown spot) on soybeans, S. tritici (Septoria blotch) on wheat and S. (syn. Stagonospora) nodorum (Stagonospora blotch) on cereals; Uncinula (syn. Erysiphe) necator (powdery mildew, anamorph: Oidium tuckeri) on vines; Setospaeria spp. (leaf blight) on corn (e. g. S. turcicum, syn. Helminthosporium turcicum) and turf; Sphacelotheca spp. (smut) on corn, (e. g. S. reiliana: head smut), sorghum und sugar cane; Sphaerotheca fuliginea (powdery mildew) on cucurbits; Spongospora subterranea (powdery scab) on potatoes and thereby transmitted viral diseases; Stagonospora spp. on cereals, e. g. S. nodorum (Stagonospora blotch, teleomorph: Leptosphaeria [syn. Phaeosphaeria] nodorum) on wheat; Synchytrium endobioticum on potatoes (potato wart disease); Taphrina spp., e. g. T. deformans (leaf curl disease) on peaches and T. pruni (plum pocket) on plums; Thielaviopsis spp. (black root rot) on tobacco, pome fruits, vegetables, soybeans and cotton, e. g. T. basicola (syn. Chalara elegans); Tilletia spp. (common bunt or stinking smut) on cereals, such as e. g. T. tritici (syn. T. caries, wheat bunt) and T. controversa (dwarf bunt) on wheat; Typhula incarnata (grey snow mold) on barley or wheat; Urocystis spp., e. g. U. occulta (stem smut) on rye; Uromyces spp. (rust) on vegetables, such as beans (e. g. U. appendiculatus, syn. U. phaseoli) and sugar beets (e. g. U. betae); Ustilago spp. (loose smut) on cereals (e. g. U. nuda and U. avaenae), corn (e. g. U. maydis: corn smut) and sugar cane; Venturia spp. (scab) on apples (e. g. V. inaequalis) and pears; and Verticilium spp. (wilt) on various plants, such as fruits and ornamentals, vines, soft fruits, vegetables and field crops, e. g. V. dahilae on strawberries, rape, potatoes and tomatoes. In a preferred embodiment the compounds I, their mixtures with other active compounds as defined herein and compositions thereof, respectively, are particularly suitable for controlling the following plant diseases: Puccinia spp. (rusts) on various plants, for example, but not limited to P. triticina (brown or leaf rust), P. striformis (stripe or yellow rust), P. hordei (dwarf rust), P. graminis (stem or black rust) or P. recondita (brown or leaf rust) on cereals, such as e. g. wheat, barley or rye and Phakopsoraceae spp. on various plants, in particular Phakopsorapachyrhizi and P. meibomiae (soybean rust) on soybeans.

The compounds I and compositions thereof, respectively, are also suitable for controlling harmful fungi in the protection of stored products or harvest and in the protection of materials. The term “protection of materials” is to be understood to denote the protection of technical and non-living materials, such as adhesives, glues, wood, paper and paperboard, textiles, leather, paint dispersions, plastics, cooling lubricants, fiber or fabrics, against the infestation and destruction by harmful microorganisms, such as fungi and bacteria. As to the protection of wood and other materials, the particular attention is paid to the following harmful fungi: Ascomycetes such as Ophiostoma spp., Ceratocystis spp., Aureobasidium pullulans, Sclerophoma spp., Chaetomium spp., Humicola spp., Petriella spp., Trichurus spp.; Basidiomycetes such as Coniophora spp., Coriolus spp., Gloeophyllum spp., Lentinus spp., Pleurotus spp., Poria spp., Serpula spp. and Tyromyces spp., Deuteromycetes such as Aspergillus spp., Cladosporium spp., Penicllium spp., Trichoderma spp., Alternaria spp., Paeciomyces spp. and Zygomycetes such as Mucor spp., and in addition in the protection of stored products and harvest the following yeast fungi are worthy of note: Candida spp. and Saccharomyces cerevisae.

The method of treatment according to the invention can also be used in the field of protecting stored products or harvest against attack of fungi and microorganisms. According to the present invention, the term “stored products” is understood to denote natural substances of plant or animal origin and their processed forms, which have been taken from the natural life cycle and for which long-term protection is desired. Stored products of crop plant origin, such as plants or parts thereof, for example stalks, leafs, tubers, seeds, fruits or grains, can be protected in the freshly harvested state or in processed form, such as pre-dried, moistened, comminuted, ground, pressed or roasted, which process is also known as post-harvest treatment. Also falling under the definition of stored products is timber, whether in the form of crude timber, such as construction timber, electricity pylons and barriers, or in the form of finished articles, such as furniture or objects made from wood. Stored products of animal origin are hides, leather, furs, hairs and the like. The combinations according the present invention can prevent disadvantageous effects such as decay, discoloration or mold. Preferably “stored products” is understood to denote natural substances of plant origin and their processed forms, more preferably fruits and their processed forms, such as pomes, stone fruits, soft fruits and citrus fruits and their processed forms.

The compounds I and compositions thereof, respectively, may be used for improving the health of a plant. The invention also relates to a method for improving plant health by treating a plant, its propagation material and/or the locus where the plant is growing or is to grow with an effective amount of compounds I and compositions thereof, respectively.

The term “plant health” is to be understood to denote a condition of the plant and/or its products which is determined by several indicators alone or in combination with each other such as yield (e. g. increased biomass and/or increased content of valuable ingredients), plant vigor (e. g. improved plant growth and/or greener leaves (“greening effect”)), quality (e. g. improved content or composition of certain ingredients) and tolerance to abiotic and/or biotic stress. The above identified indicators for the health condition of a plant may be interdependent or may result from each other.

The compounds of formula I can be present in different crystal modifications whose biological activity may differ. They are likewise subject matter of the present invention.

The compounds I are employed as such or in form of compositions by treating the fungi or the plants, plant propagation materials, such as seeds, soil, surfaces, materials or rooms to be protected from fungal attack with a fungicidally effective amount of the active substances. The application can be carried out both before and after the infection of the plants, plant propagation materials, such as seeds, soil, surfaces, materials or rooms by the fungi.

Plant propagation materials may be treated with compounds I as such or a composition comprising at least one compound I prophylactically either at or before planting or transplanting. The invention also relates to agrochemical compositions comprising an auxiliary and at least one compound I according to the invention.

An agrochemical composition comprises a fungicidally effective amount of a compound I. The term “effective amount” denotes an amount of the composition or of the compounds I, which is sufficient for controlling harmful fungi on cultivated plants or in the protection of materials and which does not result in a substantial damage to the treated plants. Such an amount can vary in a broad range and is dependent on various factors, such as the fungal species to be controlled, the treated cultivated plant or material, the climatic conditions and the specific compound I used.

The compounds I, their N-oxides and salts can be converted into customary types of agrochemical compositions, e. g. solutions, emulsions, suspensions, dusts, powders, pastes, granules, pressings, capsules, and mixtures thereof. Examples for composition types are suspensions (e. g. SC, OD, FS), emulsifiable concentrates (e. g. EC), emulsions (e. g. EW, EO, ES, ME), capsules (e. g. CS, ZC), pastes, pastilles, wettable powders or dusts (e. g. WP, SP, WS, DP, DS), pressings (e. g. BR, TB, DT), granules (e. g. WG, SG, GR, FG, GG, MG), insecticidal articles (e. g. LN), as well as gel formulations for the treatment of plant propagation materials such as seeds (e. g. GF). These and further compositions types are defined in the “Catalogue of pesticide formulation types and international coding system”, Technical Monograph No. 2, 6^{th} Ed. May 2008, CropLife International.

The compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

The binary or ternary mixtures of active compounds described herein can be prepared as compositions comprising besides the active ingredients at least one inert ingredient (auxiliary) by usual means, e. g. by the means given herein for the compositions of compounds I.

Concerning usual ingredients of such compositions reference is made to the explanations given for the compositions containing compounds I.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e. g. kerosene, diesel oil; oils of vegetable or animal origin; aliphatic, cyclic and aromatic hydrocarbons, e. g. toluene, paraffin, tetrahydronaphthalene, alkylated naphthalenes; alcohols, e. g. ethanol, propanol, butanol, benzyl alcohol, cyclohexanol; glycols; DMSO; ketones, e. g. cyclohexanone; esters, e. g. lactates, carbonates, fatty acid esters, gamma-butyrolactone; fatty acids; phosphonates; amines; amides, e. g. N-methyl pyrrolidone, fatty acid dimethyl amides; and mixtures thereof.

Suitable solid carriers or fillers are mineral earths, e. g. silicates, silica gels, talc, kaolins, limestone, lime, chalk, clays, dolomite, diatomaceous earth, bentonite, calcium sulfate, magnesium sulfate, magnesium oxide; polysaccharides, e. g. cellulose, starch; fertilizers, e. g. ammonium sulfate, ammonium phosphate, ammonium nitrate, ureas; products of vegetable origin, e. g. cereal meal, tree bark meal, wood meal, nutshell meal, and mixtures thereof.

Suitable surfactants are surface-active compounds, such as anionic, cationic, nonionic and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylaryl sulfonates, diphenyl sulfonates, alpha-olefin sulfonates, lignin sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkyl naphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids or fatty acid esters which have been alkoxylated with 1 to 50 equivalents. Ethylene oxide and/or propylene oxide may be employed for the alkoxylation, preferably ethylene oxide.

Examples of N-substituted fatty acid amides are fatty acid glucamides or fatty acid alkanolamides. Examples of esters are fatty acid esters, glycerol esters or monoglycerides.

Examples of sugar-based surfactants are sorbitans, ethoxylated sorbitans, sucrose and glucose esters or alkylpolyglucosides. Examples of polymeric surfactants are home- or copolymers of vinyl pyrrolidone, vinyl alcohols, or vinyl acetate.

Suitable cationic surfactants are quaternary surfactants, for example quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B—C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinyl amines or polyethylene amines.

Suitable adjuvants are compounds, which have a negligible or even no pesticidal activity themselves, and which improve the biological performance of the compound I on the target. Examples are surfactants, mineral or vegetable oils, and other auxiliaries. Further examples are listed by Knowles, Adjuvants and additives, Agrow Reports DS256, T&F Informa UK, 2006, chapter 5.

Suitable thickeners are polysaccharides (e. g. xanthan gum, carboxymethyl cellulose), inorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea and glycerin.

Suitable anti-foaming agents are silicones, long chain alcohols, and salts of fatty acids.

Suitable colorants (e. g. in red, blue, or green) are pigments of low water solubility and water-soluble dyes. Examples are inorganic colorants (e. g. iron oxide, titan oxide, iron hexacyanoferrate) and organic colorants (e. g. alizarin-, azo- and phthalocyanine colorants).

Suitable tackifiers or binders are polyvinyl pyrrolidones, polyvinyl acetates, polyvinyl alcohols, polyacrylates, biological or synthetic waxes, and cellulose ethers.

Examples for composition types and their preparation are:

### i) Water-Soluble Concentrates (SL, LS)

10-60 wt % of a compound I and 5-15 wt % wetting agent (e. g. alcohol alkoxylates) are dissolved in water and/or in a water-soluble solvent (e. g. alcohols) ad 100 wt %. The active compound dissolves upon dilution with water.

### ii) Dispersible Concentrates (DC)

5-25 wt % of a compound I and 1-10 wt % dispersant (e. g. polyvinyl pyrrolidone) are dissolved in organic solvent (e. g. cyclohexanone) ad 100 wt %. Dilution with water gives a dispersion.

iii) Emulsifiable Concentrates (EC)

15-70 wt % of a compound I and 5-10 wt % emulsifiers (e. g. calcium dodecylbenzenesulfonate and castor oil ethoxylate) are dissolved in water-insoluble organic solvent (e. g. aromatic hydrocarbon) ad 100 wt %. Dilution with water gives an emulsion.

### iv) Emulsions (EW, EO, ES)

5-40 wt % of a compound I and 1-10 wt % emulsifiers (e. g. calcium dodecylbenzenesulfonate and castor oil ethoxylate) are dissolved in 20-40 wt % water-insoluble organic solvent (e. g. aromatic hydrocarbon). This mixture is introduced into water ad 100 wt % by means of an emulsifying machine and made into a homogeneous emulsion. Dilution with water gives an emulsion.

### v) Suspensions (SC, OD, FS)

In an agitated ball mill, 20-60 wt % of a compound I are comminuted with addition of 2-10 wt % dispersants and wetting agents (e. g. sodium lignosulfonate and alcohol ethoxylate), 0.1-2 wt % thickener (e. g. xanthan gum) and water ad 100 wt % to give a fine active compound suspension. Dilution with water gives a stable suspension of the active substance. For FS type composition up to 40 wt % binder (e. g. polyvinyl alcohol) is added.

### vi) Water-Dispersible Granules and Water-Soluble Granules (WG, SG)

50-80 wt % of a compound I are ground finely with addition of dispersants and wetting agents (e. g. sodium lignosulfonate and alcohol ethoxylate) ad 100 wt % and prepared as water-dispersible or water-soluble granules by means of technical appliances (e. g. extrusion, spray tower, fluidized bed). Dilution with water gives a stable dispersion or solution of the active substance.

vii) Water-Dispersible Powders and Water-Soluble Powders (WP, SP, WS)

50-80 wt % of a compound I are ground in a rotor-stator mill with addition of 1-5 wt % dispersants (e. g. sodium lignosulfonate), 1-3 wt % wetting agents (e. g. alcohol ethoxylate) and solid carrier (e. g. silica gel) ad 100 wt %. Dilution with water gives a stable dispersion or solution of the active substance.

viii) Gel (GW, GF)

In an agitated ball mill, 5-25 wt % of a compound I are comminuted with addition of 3-10 wt % dispersants (e. g. sodium lignosulfonate), 1-5 wt % thickener (e. g. carboxymethyl cellulose) and water ad 100 wt % to give a fine suspension of the active substance. Dilution with water gives a stable suspension of the active substance.

### ix) Microemulsion (ME)

5-20 wt % of a compound I are added to 5-30 wt % organic solvent blend (e. g. fatty acid dimethyl amide and cyclohexanone), 10-25 wt % surfactant blend (e. g. alcohol ethoxylate and arylphenol ethoxylate), and water ad 100%. This mixture is stirred for 1 h to produce spontaneously a thermodynamically stable microemulsion.

### x) Microcapsules (CS)

An oil phase comprising 5-50 wt % of a compound I, 0-40 wt % water insoluble organic solvent (e. g. aromatic hydrocarbon), 2-15 wt % acrylic monomers (e. g. methylmethacrylate, methacrylic acid and a di- or triacrylate) are dispersed into an aqueous solution of a protective colloid (e. g. polyvinyl alcohol). Radical polymerization results in the formation of poly(meth)acrylate microcapsules. Alternatively, an oil phase comprising 5-50 wt % of a compound I according to the invention, 0-40 wt % water insoluble organic solvent (e. g. aromatic hydrocarbon), and an isocyanate monomer (e. g. diphenylmethene-4,4′-diisocyanatae) are dispersed into an aqueous solution of a protective colloid (e. g. polyvinyl alcohol). The addition of a polyamine (e. g. hexamethylenediamine) results in the formation of polyurea microcapsules. The monomers amount to 1-10 wt %. The wt % relate to the total CS composition.

### xi) Dustable Powders (DP, DS)

1-10 wt % of a compound I are ground finely and mixed intimately with solid carrier (e. g. finely divided kaolin) ad 100 wt %.

xii) Granules (GR, FG)

0.5-30 wt % of a compound I is ground finely and associated with solid carrier (e. g. silicate) ad 100 wt %. Granulation is achieved by extrusion, spray-drying or fluidized bed.

xiii) Ultra-Low Volume Liquids (UL)

1-50 wt % of a compound I are dissolved in organic solvent (e. g. aromatic hydrocarbon) ad 100 wt %.

The compositions types i) to xiii) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions generally comprise between 0.01 and 95%, preferably between 0.1 and 90%, more preferably between 1 and 70%, and in particular between 10 and 60%, by weight of active substance. The active substances are employed in a purity of from 90% to 100%, preferably from 95% to 100% (according to NMR spectrum).

For the purposes of treatment of plant propagation materials, particularly seeds, solutions for seed treatment (LS), Suspoemulsions (SE), flowable concentrates (FS), powders for dry treatment (DS), water-dispersible powders for slurry treatment (WS), water-soluble powders (SS), emulsions (ES), emulsifiable concentrates (EC), and gels (GF) are usually employed. The compositions in question give, after two-to-tenfold dilution, active compound concentrations of from 0.01 to 60% by weight, preferably from 0.1 to 40%, in the ready-to-use preparations.

Application can be carried out before or during sowing. Methods for applying compound I and compositions thereof, respectively, onto plant propagation material, especially seeds, include dressing, coating, pelleting, dusting, and soaking as well as in-furrow application methods. Preferably, compound I or the compositions thereof, respectively, are applied on to the plant propagation material by a method such that germination is not induced, e. g. by seed dressing, pelleting, coating and dusting.

When employed in plant protection, the amounts of active substances applied are, depending on the kind of effect desired, from 0.001 to 2 kg per ha, preferably from 0.005 to 2 kg per ha, more preferably from 0.05 to 0.9 kg per ha, and in particular from 0.1 to 0.75 kg per ha.

In treatment of plant propagation materials such as seeds, e. g. by dusting, coating or drenching seed, amounts of active compound of from 0.1 to 1000 g, preferably from 1 to 1000 g, more preferably from 1 to 100 g and most preferably from 5 to 100 g, per 100 kilogram of plant propagation material (preferably seeds) are generally required.

When used in the protection of materials or stored products, the amount of active compound applied depends on the kind of application area and on the desired effect. Amounts customarily applied in the protection of materials are 0.001 g to 2 kg, preferably 0.005 g to 1 kg, of active compound per cubic meter of treated material.

Various types of oils, wetters, adjuvants, fertilizer, or micronutrients, and further pesticides (e. g. herbicides, insecticides, fungicides, growth regulators, safeners, biopesticides) may be added to the active substances or the compositions comprising them as premix or, if appropriate not until immediately prior to use (tank mix). These agents can be admixed with the compositions according to the invention in a weight ratio of 1:100 to 100:1, preferably 1:10 to 10:1.

A pesticide is generally a chemical or biological agent (such as pestidal active ingredient, compound, composition, virus, bacterium, antimicrobial or disinfectant) that through its effect deters, incapacitates, kills or otherwise discourages pests. Target pests can include insects, plant pathogens, weeds, mollusks, birds, mammals, fish, nematodes (roundworms), and microbes that destroy property, cause nuisance, spread disease or are vectors for disease. The term “pesticide” includes also plant growth regulators that alter the expected growth, flowering, or reproduction rate of plants; defoliants that cause leaves or other foliage to drop from a plant, usually to facilitate harvest; desiccants that promote drying of living tissues, such as unwanted plant tops; plant activators that activate plant physiology for defense of against certain pests; safeners that reduce unwanted herbicidal action of pesticides on crop plants; and plant growth promoters that affect plant physiology e.g. to increase plant growth, biomass, yield or any other quality parameter of the harvestable goods of a crop plant.

The user applies the composition according to the invention usually from a predosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, individual components of the composition according to the invention such as parts of a kit or parts of a binary or ternary mixture may be mixed by the user himself in a spray tank or any other kind of vessel used for applications (e. g. seed treater drums, seed pelleting machinery, knapsack sprayer) and further auxiliaries may be added, if appropriate.

Consequently, one embodiment of the invention is a kit for preparing a usable pesticidal composition, the kit comprising a) a composition comprising component 1) as defined herein and at least one auxiliary; and b) a composition comprising component 2) as defined herein and at least one auxiliary; and optionally c) a composition comprising at least one auxiliary and optionally a further active component 3) as defined herein.

Mixing the compounds I or the compositions comprising them in the use form as fungicides with other fungicides results in many cases in an expansion of the fungicidal spectrum of activity being obtained or in a prevention of fungicide resistance development. Furthermore, in many cases, synergistic effects are obtained.

The present invention furthermore relates to agrochemical compositions comprising a mixture of at least one compound I (component 1) and at least one further active compound useful for plant protection, e. g. selected from the groups A) to O) (component 2), in particular one further fungicide, e. g. one or more fungicide from the groups A) to K), as described above, and if desired one suitable solvent or solid carrier. Those mixtures are of particular interest, since many of them at the same application rate show higher efficiencies against harmful fungi. Furthermore, combating harmful fungi with a mixture of compounds I and at least one fungicide from groups A) to K), as described above, is more efficient than combating those fungi with individual compounds I or individual fungicides from groups A) to K).

By applying compounds I together with at least one active compound from groups A) to O) a synergistic effect can be obtained, i.e. more than simple addition of the individual effects is obtained (synergistic mixtures).

This can be obtained by applying the compounds I and at least one further active compound simultaneously, either jointly (e. g. as tank-mix) or separately, or in succession, wherein the time interval between the individual applications is selected to ensure that the active compound applied first still occurs at the site of action in a sufficient amount at the time of application of the further active substance(s). The order of application is not essential for working of the present invention.

When applying compound I and a pesticide II sequentially the time between both applications may vary e. g. between 2 hours to 7 days. Also a broader range is possible ranging from 0.25 hour to 30 days, preferably from 0.5 hour to 14 days, particularly from 1 hour to 7 days or from 1.5 hours to 5 days, even more preferred from 2 hours to 1 day.

In the binary mixtures and compositions according to the invention the weight ratio of the component 1) and the component 2) generally depends from the properties of the active components used, usually it is in the range of from 1:10000 to 10000:1, often it is in the range of from 1:100 to 100:1, regularly in the range of from 1:50 to 50:1, preferably in the range of from 1:20 to 20:1, more preferably in the range of from 1:5 to 5:1 and in particular in the range of from 1:2 to 2:1.

According to a further embodiment of the binary mixtures and compositions, the weight ratio of the component 1) and the component 2) usually is in the range of from 1000:1 to 1:1, often in the range of from 100:1 to 1:1, regularly in the range of from 50:1 to 1:1, preferably in the range of from 20:1 to 1:1, more preferably in the range of from 5:1 to 1:1 and in particular in the range of from 2:1 to 1:1.

According to a further embodiments of the binary mixtures and compositions, the weight ratio of the component 1) and the component 2) usually is in the range of from 1:1 to 1:1000, often in the range of from 1:1 to 1:100, regularly in the range of from 1:1 to 1:50, preferably in the range of from 1:1 to 1:20, more preferably in the range of from 1:1 to 1:5 and in particular in the range of from 1:1 to 1:2.

In the ternary mixtures, i.e. compositions according to the invention comprising the component 1) and component 2) and a compound III (component 3), the weight ratio of component 1) and component 2) is in the range of from 1:100 to 100:1, regularly in the range of from 1:50 to 50:1, preferably in the range of from 1:20 to 20:1, more preferably in the range of from 1:5 to 5:1 and in particular in the range of from 1:2 to 2:1, and the weight ratio of component 1) and component 3) is in the range of from 1:100 to 100:1, regularly in the range of from 1:50 to 50:1, preferably in the range of from 1:20 to 20:1, more preferably in the range of from 1:5 to 5:1 and in particular in the range of from 1:2 to 2:1.

Any further active components are, if desired, added in a ratio of from 20:1 to 1:20 to the component 1).

These ratios are also suitable for inventive mixtures applied by seed treatment.

### Biological Activity

The fungicidal action of the mixtures according to the invention can be shown by the tests described below. The efficacy (E) is calculated as follows using Abbot's formula:

E=(1−α/β)·100
α corresponds to the fungicidal infection of the treated plants in % and
β corresponds to the fungicidal infection of the untreated (control) plants in %

An efficacy of 0 means that the infection level of the treated plants corresponds to that of the untreated control plants; an efficacy of 100 means that the treated plants were not infected. The expected efficacies of active compound combinations may be determined using Colby's formula (Colby, S.R. “Calculating synergistic and antagonistic responses of herbicide combinations”, Weeds, 15, pp. 20-22, 1967) and compared with the observed efficacies. Colby's formula:

E=x+y−x·y/100
E expected efficacy, expressed in % of the untreated control, when using the mixture of the active compounds A and B at the concentrations a and b
x efficacy, expressed in % of the untreated control, when using the active compound A at the concentration a
y efficacy, expressed in % of the untreated control, when using the active compound B at the concentration b.

The expression “I.B/I.C” relates to a mixture of stereoisomers 1.B and I.C.

The expression “I.H/I.J” relates to a mixture of stereoisomers I.H and I.J.

I) Fungicidal Activity Against Botrytis cinerea

The active compounds were formulated separately as a stock solution having a concentration of 10000 ppm in dimethyl sulfoxide. The stock solutions were mixed according to the ratio, pipetted onto a micro titer plate (MTP) and diluted with water to the stated concentrations. A spore suspension of Botrytis cinerea in an aqueous biomalt or yeast-bactopeptone-sodiumacetate solution was then added. The plates were placed in a water vapor-saturated chamber at a temperature of 18° C. Using an absorption photometer, the MTPs were measured at 405 nm 7 days after the inoculation. The measured parameters were compared to the growth of the active compound-free control variant (100%) and the fungus-free and active compound-free blank value to determine the relative growth in % of the pathogens in the respective active compounds. These percentages were converted into efficacies.

TABLE I.1
Use examples of fungicidal activity against Botrytis cinerea
of I.H/I.J as component A and components B

| Active mixture | Code of | Concentration | Efficacy |
|---|---|---|---|
| (component A + B) | component B | A + B (ppm) | (ppm) |
| I.H/I.J + component B | A.3.29 | 16 + 1 | 86 |
| I.H/I.J + component B | K.1.29 | 16 + 16 | 99 |
| I.H/I.J + component B | A.2.5 | 16 + 1 | 81 |
| I.H/I.J + component B | A.3.32 | 16 + 1 | 81 |
| I.H/I.J + component B | K.1.30 | 16 + 4 | 99 |
| I.H/I.J + component B | K.1.32 | 16 + 1 | 89 |
| I.H/I.J + component B | K.1.31 | 16 + 4 | 99 |
| I.H/I.J + Benomyl | D.1.1 | 16 + 4 | 100 |
| I.H/I.J + Bixafen | A.3.3 | 16 + 0.25 | 59 |
| I.H/I.J + Boscalid | A.3.4 | 16 + 4 | 85 |
| I.H/I.J + Captan | H.3.4 | 16 + 4 | 100 |
| I.H/I.J + Carbendazim | D.1.2 | 16 + 0.25 | 100 |
| I.H/I.J + Cyproconazol | B.1.4 | 16 + 16 | 100 |
| I.H/I.J + Cyprodinil | E.1.1 | 16 + 4 | 72 |
| I.H/I.J + Dithianon | H.4.9 | 16 + 1 | 100 |
| I.H/I.J + Epoxiconazol | B.1.8. | 16 + 0.25 | 72 |
| I.H/I.J + Fenpropimorph | B.2.4 | 16 + 1 | 91 |
| I.H/I.J + Fludioxonil | F.1.5 | 16 + 0.25 | 98 |
| I.H/I.J + Fluindapyr | A.3.28 | 16 + 1 | 78 |
| I.H/I.J + Fluquinconazol | B.1.10 | 16 + 1 | 100 |
| I.H/I.J + Penthiopyrad | A.3.16 | 16 + 0.25 | 51 |
| I.H/I.J + Pydiflumetofen | A.3.17 | 16 + 16 | 96 |
| I.H/I.J + Tebuconazol | B.1.25 | 16 + 0.25 | 54 |

TABLE I.2
Use examples of fungicidal activity against Botrytis
cinerea of I.F as component A and components B

| Active mixture | Code of | Concentration | Efficacy |
|---|---|---|---|
| (component A + B) | component B | (ppm) | (ppm) |
| I.F + component B | A.3.29 | 4 + 1 | 87 |
| I.F + component B | A.2.5 | 4 + 1 | 51 |
| I.F + component B | A.3.32 | 4 + 1 | 80 |
| I.F + Benomyl | D.1.1 | 4 + 4 | 100 |
| I.F + Bixafen | A.3.3 | 4 + 0.25 | 58 |
| I.F + Boscalid | A.3.4 | 4 + 4 | 84 |
| I.F + Captan | H.3.4 | 4 + 4 | 100 |
| I.F + Carbendazim | D.1.2 | 4 + 0.25 | 100 |
| I.F + Cyproconazol | B.1.4 | 4 + 16 | 100 |
| I.F + Cyprodinil | E.1.1 | 4 + 4 | 100 |
| I.F + Dithianon | H.4.9 | 4 + 1 | 100 |
| I.F + Epoxiconazol | B.1.8. | 4 + 0.25 | 79 |
| I.F + Fenpropimorph | B.2.4 | 4 + 1 | 98 |
| I.F + Fludioxonil | F.1.5 | 4 + 0.25 | 100 |
| I.F + Fluindapyr | A.3.28 | 4 + 1 | 74 |
| I.F + Fluquinconazol | B.1.10 | 4 + 1 | 100 |
| I.F + component B | A.3.24 | 4 + 1 | 82 |

TABLE I.3
Use examples of fungicidal activity against Botrytis
cinerea of I.B/I.C as component A and components B

| Active mixture | Code of | Concentration | Efficacy |
|---|---|---|---|
| (component A + B) | component B | (ppm) | (ppm) |
| I.B/I.C + component B | A.3.29 | 1 + 1 | 86 |
| I.B/I.C + component B | K.1.29 | 1 + 16 | 100 |
| I.B/I.C + component B | A.2.5 | 1 + 1 | 62 |
| I.B/I.C + component B | A.3.32 | 1 + 1 | 79 |
| I.B/I.C + component B | K.1.30 | 1 + 4 | 100 |
| I.B/I.C + component B | K.1.32 | 1 + 1 | 94 |
| I.B/I.C + component B | K.1.31 | 1 + 4 | 100 |
| I.B/I.C + Benomyl | D.1.1 | 1 + 4 | 100 |
| I.B/I.C + Bixafen | A.3.3 | 1 + 0.25 | 58 |
| I.B/I.C + Boscalid | A.3.4 | 1 + 4 | 84 |
| I.B/I.C + Captan | H.3.4 | 1 + 4 | 100 |
| I.B/I.C + Carbendazim | D.1.2 | 1 + 0.25 | 100 |
| I.B/I.C + Cyproconazol | B.1.4 | 1 + 16 | 100 |
| I.B/I.C + Cyprodinil | E.1.1 | 1 + 4 | 100 |
| I.B/I.C + Dithianon | H.4.9 | 1 + 1 | 100 |
| I.B/I.C + Epoxiconazol | B.1.8. | 1 + 0.25 | 77 |
| I.B/I.C + Fenpropimorph | B.2.4 | 1 + 1 | 98 |
| I.B/I.C + Fludioxonil | F.1.5 | 1 + 0.25 | 99 |
| I.B/I.C + Fluindapyr | A.3.28 | 1 + 1 | 73 |
| I.B/I.C + Pydiflumetofen | A.3.17 | 1 + 16 | 96 |
| I.B/I.C + Tebuconazol | B.1.25 | 1 + 0.25 | 57 |

TABLE I.4
Use examples fungicidal activity against
Botrytis cinerea of selected components B

| Code of | Common name | Concentration | Observed |
|---|---|---|---|
| component B | component B | (ppm) | efficacy |
| A.3.29 | | 0.25 | 55 |
| A.2.3 | | 1 | 11 |
| | | 0.25 | 0 |
| A.2.5 | | 1 | 6 |
| A.3.34 | | 1 | 51 |
| | | 0.25 | 1 |
| A.3.32 | | 0.25 | 32 |
| K.1.31 | | 1 | 53 |
| A.1.34 | | 63 | 18 |
| | | 16 | 2 |
| C.1.2 | Benalaxyl-M | 4 | 7 |
| D.1.1 | Benomyl | 0.016 | 8 |
| A.3.2 | Benzovindiflupyr | 0.063 | 27 |
| A.3.3 | Bixafen | 0.25 | 53 |
| | | 0.063 | 10 |
| H.3.4 | Captan | 1 | 73 |
| | | 0.25 | 20 |
| H.3.2 | Chlorothalonil | 1 | 22 |
| E.1.1 | Cyprodinil | 1 | 66 |
| A.1.4 | Dimoxystrobin | 1 | 14 |
| | | 0.25 | 2 |
| B.2.6 | Fenpropidin | 4 | 28 |
| B.2.4 | Fenpropimorph | 0.25 | 29 |
| F.1.5 | Fludioxonil | 0.016 | 77 |
| A.3.28 | Fluindapyr | 0.25 | 21 |
| H.3.5 | Folpet | 1 | 13 |
| A.3.24 | | 0.25 | 41 |
| A.3.29 | Isoflucypram | 0.25 | 53 |
| B.1.38 | Mefentrifluconazol | 0.016 | 50 |
| A.1.13 | Picoxystrobin | 0.25 | 9 |
| A.1.14 | Pyraclostrobin | 0.016 | 14 |
| | | 0.004 | 0 |
| A.3.17 | Pyriofenon | 1 | 2 |
| A.1.17 | Trifloxystrobin | 0.063 | 3 |

TABLE I.5
Use examples of synergistic fungicidal activity against Botrytis
cinerea of compound I.M as component A and components B

| | | | | Calculated | |
|---|---|---|---|---|---|
| Active | | | | efficacy | |
| compound/ | Concen- | | | according | Syner- |
| active mixture | tration | Ratio | Observed | to Colby | gism |
| A + B | (ppm) | A:B | efficacy | (%) | (%) |
| I.M | 4 | — | 15 | | |
| | 1 | — | 0 | | |
| | 0.25 | — | 2 | | |
| | 0.063 | — | 1 | | |
| I.M | 1 | 1:1 | 33 | 11 | 22 |
| A.2.3 | 1 | | | | |
| I.M | 4 | 4:1 | 91 | 24 | 67 |
| A.2.3 | 1 | | | | |
| I.M | 4 | 16:1 | 54 | 15 | 39 |
| A.2.3 | 0.25 | | | | |
| I.M | 4 | 4:1 | 49 | 20 | 29 |
| A.2.5 | 1 | | | | |
| I.M | 4 | 4:1 | 77 | 58 | 19 |
| A.3.34 | 1 | | | | |
| I.M | 4 | 16:1 | 41 | 15 | 26 |
| A.3.34 | 0.25 | | | | |
| I.M | 1 | 1:63 | 55 | 19 | 36 |
| A.1.34 | 63 | | | | |
| I.M | 4 | 1:4 | 99 | 16 | 83 |
| A.1.34 | 16 | | | | |
| I.M | 1 | 1:4 | 99 | 7 | 92 |
| Benalaxyl-M | 4 | | | | |
| I.M | 4 | 1:63 | 59 | 37 | 22 |
| Benzovindiflupyr | 0.063 | | | | |
| I.M | 4 | 16:1 | 84 | 60 | 24 |
| Bixafen | 0.25 | | | | |
| I.M | 0.25 | 1:1 | 72 | 21 | 51 |
| Captan | 0.25 | | | | |
| I.M | 0.25 | 1:4 | 100 | 74 | 26 |
| Captan | 1 | | | | |
| I.M | 4 | 4:1 | 100 | 77 | 23 |
| Captan | 1 | | | | |
| I.M | 4 | 4:1 | 92 | 33 | 59 |
| Chlorothalonil | 1 | | | | |
| I.M | 1 | 1:1 | 57 | 22 | 35 |
| Chlorothalonil | 1 | | | | |
| I.M | 4 | 4:1 | 88 | 27 | 61 |
| Dimoxystrobin | 1 | | | | |
| I.M | 1 | 1:1 | 51 | 15 | 36 |
| Dimoxystrobin | 1 | | | | |
| I.M | 4 | 16:1 | 50 | 16 | 34 |
| Dimoxystrobin | 0.25 | | | | |
| I.M | 1 | 1:4 | 67 | 29 | 38 |
| Fenpropidin | 4 | | | | |
| I.M | 4 | 1:1 | 95 | 39 | 56 |
| Fenpropidin | 4 | | | | |
| I.M | 1 | 4:1 | 66 | 29 | 37 |
| Fenpropimorph | 0.25 | | | | |
| I.M | 0.063 | 4:1 | 96 | 77 | 19 |
| Fludioxonil | 0.016 | | | | |
| I.M | 4 | 16:1 | 54 | 33 | 21 |
| Fluindapyr | 0.25 | | | | |
| I.M | 4 | 16:1 | 79 | 60 | 19 |
| Isoflucypram | 0.25 | | | | |
| I.M | 4 | 250:1 | 100 | 58 | 42 |
| Mefentrifluconazol | 0.016 | | | | |
| I.M | 1 | 63:1 | 100 | 51 | 49 |
| Mefentrifluconazol | 0.016 | | | | |
| I.M | 4 | 16:1 | 70 | 22 | 48 |
| Picoxystrobin | 0.25 | | | | |
| I.M | 1 | 4:1 | 37 | 9 | 28 |
| Picoxystrobin | 0.25 | | | | |
| I.M | 4 | 63:1 | 40 | 15 | 25 |
| Picoxystrobin | 0.063 | | | | |
| I.M | 4 | 1000:1 | 57 | 15 | 42 |
| Pyraclostrobin | 0.004 | | | | |
| I.M | 1 | 63:1 | 57 | 14 | 43 |
| Pyraclostrobin | 0.016 | | | | |
| I.M | 4 | 250:1 | 91 | 26 | 65 |
| Pyraclostrobin | 0.016 | | | | |
| I.M | 4 | 63:1 | 36 | 17 | 19 |
| Trifloxystrobin | 0.063 | | | | |

TABLE I.6
Use examples of synergistic fungicidal activity against Botrytis
cinerea of compound I.E as component A and components B

| | | | | Calculated | |
|---|---|---|---|---|---|
| Active | | | | efficacy | |
| compound/ | Concen- | | | according | Syner- |
| active mixture | tration | Ratio | Observed | to Colby | gism |
| A + B | (ppm) | A:B | efficacy | (%) | (%) |
| I.E | 63 | — | 16 | | |
| | 16 | — | 3 | | |
| | 4 | — | 2 | | |
| | 1 | — | 2 | | |
| | 0.063 | — | 2 | | |
| I.E | 0.063 | 4:1 | 33 | 10 | 23 |
| Benomyl | 0.016 | | | | |
| I.E | 16 | 250:1 | 61 | 35 | 26 |
| Benzovindiflupyr | 0.063 | | | | |
| I.E | 16 | 63:1 | 81 | 58 | 23 |
| Bixafen | 0.25 | | | | |
| I.E | 4 | 16:1 | 49 | 22 | 27 |
| Captan | 0.25 | | | | |
| I.E | 16 | 63:1 | 53 | 23 | 30 |
| Captan | 0.25 | | | | |
| I.E | 63 | 63:1 | 99 | 71 | 28 |
| Cyprodinil | 1 | | | | |
| I.E | 16 | 4:1 | 63 | 37 | 26 |
| Fenpropidin | 4 | | | | |
| I.E | 16 | 63:1 | 83 | 31 | 52 |
| Fenpropimorph | 0.25 | | | | |
| I.E | 4 | 16:1 | 56 | 30 | 26 |
| Fenpropimorph | 0.25 | | | | |
| I.E | 16 | 16:1 | 88 | 16 | 72 |
| Folpet | 1 | | | | |
| I.E | 63 | 4000:1 | 92 | 58 | 34 |
| Mefentrifluconazol | 0.016 | | | | |
| I.E | 16 | 1000:1 | 95 | 52 | 43 |
| Mefentrifluconazol | 0.016 | | | | |

TABLE I.7
Use examples of synergistic fungicidal activity against Botrytis
cinerea of compound I.A as component A and components B

| | | | | Calculated | |
|---|---|---|---|---|---|
| Active | | | | efficacy | |
| compound/ | Concen- | | | according | Syner- |
| active mixture | tration | Ratio | Observed | to Colby | gism |
| A + B | (ppm) | A:B | efficacy | (%) | (%) |
| I.A | 16 | — | 12 | | |
| | 4 | — | 1 | | |
| | 0.25 | — | 3 | | |
| I.A | 16 | 63:1 | 84 | 60 | 24 |
| A.3.29 | 0.25 | | | | |
| I.A | 16 | 63:1 | 49 | 13 | 36 |
| A.3.34 | 0.25 | | | | |
| I.A | 16 | 16:1 | 79 | 57 | 22 |
| A.3.34 | 1 | | | | |
| I.A | 16 | 63:1 | 72 | 41 | 31 |
| A.3.32 | 0.25 | | | | |
| I.A | 16 | 16:1 | 91 | 61 | 30 |
| K.1.31 | 1 | | | | |
| I.A | 16 | 1:1 | 45 | 14 | 31 |
| A.1.34 | 16 | | | | |
| I.A | 16 | 250:1 | 61 | 35 | 26 |
| Benzovindiflupyr | 0.063 | | | | |
| I.A | 16 | 63:1 | 81 | 58 | 23 |
| Bixafen | 0.25 | | | | |
| I.A | 16 | 250:1 | 54 | 21 | 33 |
| Bixafen | 0.063 | | | | |
| I.A | 4 | 4:1 | 97 | 74 | 23 |
| Captan | 1 | | | | |
| I.A | 0.25 | 1:1 | 50 | 23 | 27 |
| Captan | 0.25 | | | | |
| I.A | 16 | 16:1 | 100 | 77 | 23 |
| Captan | 1 | | | | |
| I.A | 16 | 16:1 | 90 | 70 | 20 |
| Cyprodinil | 1 | | | | |
| I.A | 16 | 4:1 | 63 | 37 | 26 |
| Fenpropidin | 4 | | | | |
| I.A | 16 | 63:1 | 66 | 31 | 35 |
| Fluindapyr | 0.25 | | | | |
| I.A | 16 | 63:1 | 79 | 49 | 30 |
| A.3.24 | 0.25 | | | | |
| I.A | 16 | 63:1 | 82 | 59 | 23 |
| Isoflucypram | 0.25 | | | | |
| I.A | 4 | 250:1 | 93 | 51 | 42 |
| Mefentrifluconazol | 0.016 | | | | |
| I.A | 16 | 250:1 | 36 | 16 | 20 |
| Penthiopyrad | 0.063 | | | | |
| I.A | 16 | 1000:1 | 59 | 24 | 35 |
| Pyraclostrobin | 0.016 | | | | |
| I.A | 4 | 4:1 | 23 | 3 | 20 |
| Pyriofenon | 1 | | | | |

1) Fungicidal Activity Against Colletaichum orbiculae in the Microtiterplate Test

The active compounds were formulated separately as a stock solution having a concentration of 10000 ppm in dimethyl sulfoxide. The stock solutions were mixed according to the ratio, pipetted onto a micro titer plate (MTP) and diluted with water to the stated concentrations. spore suspension of Colletotrichum orbiculare in an aqueous biomalt or yeast-bactopeptone-sodiumacetate solution was then added. The plates were placed in a water vapor-saturated chamber at a temperature of 18° C. Using an absorption photometer, the MTPs were measured at 405 nm days after the inoculation. The measured parameters were compared to the growth of the active compound-free control variant (100%) and the fungus-free and active compound-free blank value to determine the relative growth in % of the pathogens in the respective active compounds. These percentages were converted into efficacies.

TABLE II.1
Use examples of fungicidal activity against Colletotrichum
orbiculare of I.H/I.J as component A and components B

| | | Concentration | |
|---|---|---|---|
| Active mixture | Code of | A + B | Efficacy |
| (component A + B) | component B | (ppm) | (ppm) |
| I.H/I.J + component B | A.1.34 | 16 + 63 | 82 |
| I.H/I.J + component B | K.1.42 | 16 + 1 | 55 |
| I.H/I.J + component B | A.1.25 | 16 + 0.016 | 53 |
| I.H/I.J + component B | K.1.53 | 16 + 1 | 81 |
| I.H/I.J + component B | A.2.3 | 16 + 1 | 80 |
| I.H/I.J + component B | A.2.5 | 16 + 1 | 77 |
| I.H/I.J + component B | K.1.29 | 16 + 16 | 100 |
| I.H/I.J + component B | A.3.32 | 16 + 1 | 66 |
| I.H/I.J + component B | K.1.30 | 16 + 4 | 100 |
| I.H/I.J + component B | K.1.32 | 16 + 1 | 100 |
| I.H/I.J + component B | K.1.31 | 16 + 4 | 100 |
| I.H/I.J + Ametoctradin | A.4.11 | 16 + 63 | 54 |
| I.H/I.J + Azoxystrobin | A.1.1 | 16 + 0.063 | 58 |
| I.H/I.J + Benalaxyl | C.1.1 | 16 + 4 | 59 |
| I.H/I.J + Benomyl | D.1.1 | 16 + 4 | 100 |
| I.H/I.J + Benzovindiflupyr | A.3.2 | 16 + 0.063 | 88 |
| I.H/I.J + Bixafen | A.3.3 | 16 + 0.25 | 62 |
| I.H/I.J + Boscalid | A.3.4 | 16 + 4 | 58 |
| I.H/I.J + Captan | H.3.4 | 16 + 4 | 99 |
| I.H/I.J + Carbendazim | D.1.2 | 16 + 0.25 | 99 |
| I.H/I.J + Chlorothalonil | H.3.2 | 16 + 1 | 99 |
| I.H/I.J + Cyazofamid | A.2.1 | 16 + 16 | 83 |
| I.H/I.J + Cyflufenamid | K.1.3 | 16 + 63 | 57 |
| I.H/I.J + Dimethomorph | G.3.1 | 16 + 4 | 66 |
| I.H/I.J + Dimoxystrobin | A.1.4 | 16 + 1 | 67 |
| I.H/I.J + Dithianon | H.4.9 | 16 + 1 | 100 |
| I.H/I.J + Fenpropidin | B.2.6 | 16 + 4 | 71 |
| I.H/I.J + Fenpropimorph | B.2.4 | 16 + 1 | 60 |
| I.H/I.J + Florylpicoxamid | A.2.6 | 16 + 1 | 55 |
| I.H/I.J + Fludioxonil | F.1.5 | 16 + 97 | 97 |
| I.H/I.J + Fluopyram | A.3.7 | 16 + 0.25 | 59 |
| I.H/I.J + Kresoxim-methyl | A.1.9 | 16 + 1 | 61 |
| I.H/I.J + Mefentrifluconazol | B.1.38 | 16 + 0.016 | 67 |
| I.H/I.J + Metconazol | B.1.17 | 16 + 0.063 | 53 |
| I.H/I.J + Metominostrobin | A.1.11 | 16 + 4 | 71 |
| I.H/I.J + Metrafenone | D.2.6 | 16 + 16 | 54 |
| I.H/I.J + Penthiopyrad | A.3.16 | 16 + 0.25 | 52 |
| I.H/I.J + Picoxystrobin | A.1.13 | 16 + 0.25 | 72 |
| I.H/I.J + Pydiflumetofen | A.3.17 | 16 + 16 | 85 |
| I.H/I.J + Pyraclostrobin | A.1.14 | 16 + 0.016 | 54 |
| I.H/I.J + Pyriofenone | A.3.17 | 16 + 1 | 66 |
| I.H/I.J + Sulfur | H.1.7 | 16 + 63 | 100 |
| I.H/I.J + Trifloxystrobin | A.1.17 | 16 + 0.063 | 57 |

TABLE II.2
Use examples of fungicidal activity against Colletotrichum
orbiculare of I.F as component A and components B

| | | Concentration | |
|---|---|---|---|
| Active mixture | Code of | A + B | Efficacy |
| (component A + B) | component B | (ppm) | (ppm) |
| I.F + component B | A.1.34 | 0.016 + 63 | 83 |
| I.F + component B | K.1.53 | 0.016 + 1 | 76 |
| I.F + component B | A.2.3 | 0.016 + 1 | 91 |
| I.F + component B | A.2.5 | 0.016 + 1 | 82 |
| I.F + component B | K.1.29 | 0.016 + 16 | 100 |
| I.F + component B | K.1.30 | 0.016 + 4 | 100 |
| I.F + component B | K.1.32 | 0.016 + 1 | 100 |
| I.F + component B | K.1.31 | 0.016 + 4 | 100 |
| I.F + Benomyl | D.1.1 | 0.016 + 4 | 100 |
| I.F + Benzovindiflupyr | A.3.2 | 0.016 + 0.063 | 57 |
| I.F + Captan | H.3.4 | 0.016 + 4 | 100 |
| I.F + Carbendazim | D.1.2 | 0.016 + 0.25 | 100 |
| I.F + Chlorothalonil | H.3.2 | 0.016 + 1 | 89 |
| I.F + Cyazofamid | A.2.1 | 0.016 + 16 | 100 |
| I.F + Dimoxystrobin | A.1.4 | 0.016 + 1 | 71 |
| I.F + Dithianon | H.4.9 | 0.016 + 1 | 100 |
| I.F + Fenpropidin | B.2.6 | 0.016 + 4 | 61 |
| I.F + Florylpicoxamid | A.2.6 | 0.016 + 1 | 83 |
| I.F + Fludioxonil | F.1.5 | 0.016 + 0.25 | 100 |
| I.F + Fluoxastrobin | A.1.8 | 0.016 + 0.25 | 62 |
| I.F + Kresoxim-methyl | A.1.9 | 0.016 + 1 | 80 |
| I.F + Mancozeb | H.2.2 | 0.016 + 1 | 54 |
| I.F + Metominostrobin | A.1.11 | 0.016 + 4 | 55 |
| I.F + Picoxystrobin | A.1.13 | 0.016 + 0.25 | 74 |
| I.F + Pydiflumetofen | A.3.17 | 0.016 + 16 | 92 |
| I.F + Sulfur | H.1.7 | 0.016 + 63 | 85 |
| I.F + Trifloxystrobin | A.1.17 | 0.016 + 0.063 | 59 |

TABLE II.3
Use examples of fungicidal activity against Colletotrichum
orbiculare of I.B/I.C as component A and components B

| | | Concentration | |
|---|---|---|---|
| Active mixture | Code of | A + B | Efficacy |
| (component A + B) | component B | (ppm) | (ppm) |
| I.B/I.C + component B | A.1.34 | 1 + 63 | 77 |
| I.B/I.C + component B | K.1.42 | 1 + 1 | 69 |
| I.B/I.C + component B | A.1.25 | 1 + 0.016 | 51 |
| I.B/I.C + component B | A.3.29 | 1 + 1 | 65 |
| I.B/I.C + component B | K.1.53 | 1 + 1 | 92 |
| I.B/I.C + component B | A.2.3 | 1 + 1 | 88 |
| I.B/I.C + component B | A.2.5 | 1 + 1 | 83 |
| I.B/I.C + component B | K.1.29 | 1 + 16 | 100 |
| I.B/I.C + component B | A.3.32 | 1 + 1 | 57 |
| I.B/I.C + component B | K.1.30 | 1 + 4 | 100 |
| I.B/I.C + component B | K.1.32 | 1 + 1 | 100 |
| I.B/I.C + component B | K.1.31 | 1 + 4 | 100 |
| I.B/I.C + component B | K.1.54 | 1 + 1 | 67 |
| I.B/I.C + Ametoctradin | A.4.11 | 1 + 63 | 64 |
| I.B/I.C + Azoxystrobin | A.1.1 | 1 + 0.063 | 65 |
| I.B/I.C + Benalaxyl | C.1.1 | 1 + 4 | 56 |
| I.B/I.C + Benalaxyl-M | C.1.2 | 1 + 4 | 59 |
| I.B/I.C + Benomyl | D.1.1 | 1 + 4 | 96 |
| I.B/I.C + Boscalid | A.3.4 | 1 + 4 | 63 |
| I.B/I.C + Captan | H.3.4 | 1 + 4 | 96 |
| I.B/I.C + Carbendazim | D.1.2 | 1 + 0.25 | 95 |
| I.B/I.C + Chlorothalonil | H.3.2 | 1 + 1 | 96 |
| I.B/I.C + Cyazofamid | A.2.1 | 1 + 16 | 95 |
| I.B/I.C + Cyflufenamid | K.1.3 | 1 + 63 | 79 |
| I.B/I.C + Cyproconazol | B.1.4 | 1 + 16 | 60 |
| I.B/I.C + Difenoconazol | B.1.5 | 1 + 0.25 | 50 |
| I.B/I.C + Dimethomorph | G.3.1 | 1 + 4 | 57 |
| I.B/I.C + Dimoxystrobin | A.1.4 | 1 + 1 | 75 |
| I.B/I.C + Dithianon | H.4.9 | 1 + 1 | 95 |
| I.B/I.C + Epoxiconazol | B.1.8. | 1 + 0.25 | 70 |
| I.B/I.C + Fenpropidin | B.2.6 | 1 + 4 | 78 |
| I.B/I.C + Florylpicoxamid | A.2.6 | 1 + 1 | 82 |
| I.B/I.C + Fludioxonil | F.1.5 | 1 + 0.25 | 95 |
| I.B/I.C + Fluindapyr | A.3.28 | 1 + 1 | 59 |
| I.B/I.C + Fluopyram | A.3.7 | 1 + 0.25 | 72 |
| I.B/I.C + Fluoxastrobin | A.1.8 | 1 + 0.25 | 63 |
| I.B/I.C + Fluquinconazol | B.1.10 | 1 + 1 | 70 |
| I.B/I.C + Fluxapyroxad | A.3.9 | 1 + 0.063 | 65 |
| I.B/I.C + component B | A.3.24 | 1 + 1 | 64 |
| I.B/I.C + Kresoxim-methyl | A.1.9 | 1 + 1 | 69 |
| I.B/I.C + Mancozeb | H.2.2 | 1 + 1 | 76 |
| I.B/I.C + Mefentrifluconazol | B.1.38 | 1 + 0.016 | 67 |
| I.B/I.C + Metconazol | B.1.17 | 1 + 0.063 | 63 |
| I.B/I.C + Metominostrobin | A.1.11 | 1 + 4 | 68 |
| I.B/I.C + Penthiopyrad | A.3.16 | 1 + 0.25 | 70 |
| I.B/I.C + Picoxystrobin | A.1.13 | 1 + 0.25 | 74 |
| I.B/I.C + Propiconazol | B.1.22 | 1 + 0.25 | 53 |
| I.B/I.C + Prothioconazol | B.1.23 | 1 + 0.25 | 52 |
| I.B/I.C + Pydiflumetofen | A.3.17 | 1 + 16 | 80 |
| I.B/I.C + Pyriofenone | A.3.17 | 1 + 1 | 68 |
| I.B/I.C + Sulfur | H.1.7 | 1 + 63 | 100 |
| I.B/I.C + Tebuconazol | B.1.25 | 1 + 0.25 | 67 |
| I.B/I.C + Trifloxystrobin | A.1.17 | 1 + 0.063 | 62 |
| I.B/I.C + Triticonazol | B.1.29 | 1 + 1 | 93 |

TABLE II.4
Use examples fungicidal activity against Colletotrichum
orbiculare of selected components B

| Code of | Common name | Concentration | Observed |
|---|---|---|---|
| component B | component B | (ppm) | efficacy |
| K.1.42 | | 1 | 21 |
| | | 0.25 | 3 |
| A.3.29 | | 1 | 11 |
| A.2.3 | | 0.25 | 77 |
| | | 0.063 | 47 |
| A.2.5 | | 0.25 | 72 |
| A.3.34 | | 1 | 14 |
| A.3.32 | | 1 | 15 |
| K.1.32 | | 0.25 | 26 |
| A.1.34 | | 0.000063 | 37 |
| A.1.25 | | 0.016 | 13 |
| A.4.11 | Ametoctradin | 63 | 14 |
| A.1.1 | Azoxystrobin | 0.016 | 5 |
| | | 0.004 | 1 |
| C.1.1 | Benalaxyl | 4 | 13 |
| C.1.2 | Benalaxyl-M | 1 | 2 |
| D.1.1 | Benomyl | 9 | 38 |
| A.3.2 | Benzovindiflupyr | 0.016 | 3 |
| | | 0.004 | 2 |
| A.3.3 | Bixafen | 0.063 | 3 |
| A.3.4 | Boscalid | 1 | 0 |
| | | 0.25 | 0 |
| H.3.4 | Captan | 0.25 | 21 |
| | | 0.001 | |
| D.1.2 | Carbendazim | 0.063 | 60 |
| K.1.3 | Cyflufenamid | 16 | 11 |
| B.1.4 | Cyproconazol | 4 | 4 |
| E.1.1 | Cyprodinil | 4 | 38 |
| | | 1 | 35 |
| B.1.5 | Difenoconazol | 0.25 | 14 |
| | | 0.016 | 1 |
| G.3.1 | Dimethomorph | 4 | 21 |
| A.1.4 | Dimoxystrobin | 1 | 71 |
| H.4.9 | Dithianon | 0.063 | 21 |
| B.1.8. | Epoxiconazol | 0.25 | 17 |
| | | 0.016 | 2 |
| B.2.6 | Fenpropidin | 4 | 47 |
| | | 1 | 9 |
| B.2.4 | Fenpropimorph | 1 | 19 |
| | | 0.25 | 5 |
| F.1.5 | Fludioxonil | | |
| A.3.28 | Fluindapyr | 1 | 12 |
| A.3.7 | Fluopyram | 0.25 | 15 |
| A.1.8 | Fluoxastrobin | 0.063 | 51 |
| B.1.10 | Fluquinconazol | 1 | 12 |
| | | 0.063 | 3 |
| A.3.9 | Fluxapyroxad | 0.063 | 10 |
| | | 0.004 | 1 |
| H.3.5 | Folpet | 1 | 20 |
| A.3.24 | | 1 | 12 |
| | | 0.25 | 3 |
| A.3.29 | Isoflucypram | 1 | 11 |
| | | 0.25 | 4 |
| A.1.9 | Kresoxim-methyl | 0.25 | 45 |
| H.2.2 | Mancozeb | 1 | 24 |
| | | 0.25 | 1 |
| B.1.38 | Mefentrifluconazol | 0.016 | 14 |
| | | 0.001 | 1 |
| B.1.17 | Metconazol | 0.063 | 11 |
| D.2.6 | Metrafenone | 4 | 14 |
| A.3.16 | Penthiopyrad | 0.25 | 8 |
| A.1.13 | Picoxystrobin | 0.25 | 63 |
| | | 0.016 | 5 |
| B.1.22 | Propiconazol | 0.25 | 15 |
| | | 0.016 | 3 |
| | | 0.063 | 3 |
| B.1.23 | Prothioconazol | 0.25 | 14 |
| A.1.14 | Pyraclostrobin | 0.016 | 60 |
| | | 0.001 | 32 |
| B.1.25 | Tebuconazol | 0.25 | 14 |
| | | 0.063 | 2 |
| A.1.17 | Trifloxystrobin | 0.016 | 9 |
| B.1.29 | Triticonazol | 1 | 11 |

TABLE II.5
Use examples of synergistic fungicidal activity against Colletotrichum
orbiculare of compound I.M as component A and components B

| | | | | Calculated | |
|---|---|---|---|---|---|
| Active | | | | efficacy | |
| compound/ | Concen- | | | according | Syner- |
| active mixture | tration | Ratio | Observed | to Colby | gism |
| A + B | (ppm) | A:B | efficacy | (%) | (%) |
| I.M | 0.016 | — | 7 | | |
| | 0.004 | — | 1 | | |
| | 0.001 | — | 1 | | |
| | 0.00025 | — | 2 | | |
| | 0.000063 | — | 2 | | |
| I.M | 0.016 | 1:16 | 30 | 10 | 20 |
| K.1.42 | 0.25 | | | | |
| I.M | 0.016 | 1:16 | 98 | 79 | 19 |
| A.2.3 | 0.25 | | | | |
| I.M | 0.001 | 1:63 | 75 | 48 | 27 |
| A.2.3 | 0.063 | | | | |
| I.M | 0.016 | 1:16 | 92 | 74 | 18 |
| A.2.5 | 0.25 | | | | |
| I.M | 0.016 | 1:16 | 73 | 31 | 42 |
| K.1.32 | 0.25 | | | | |
| I.M | 0.000063 | 1:1000 | 56 | 38 | 18 |
| A.1.34 | 0.063 | | | | |
| I.M | 0.016 | 1:1 | 39 | 17 | 22 |
| Azoxystrobin | 0.016 | | | | |
| I.M | 0.00025 | 1:250 | 70 | 39 | 31 |
| Benomyl | 0.063 | | | | |
| I.M | 0.000063 | 1:1000 | 57 | 39 | 18 |
| Benomyl | 0.063 | | | | |
| I.M | 0.001 | 1:63 | 70 | 39 | 31 |
| Benomyl | 0.063 | | | | |
| I.M | 0.016 | 1:1 | 29 | 10 | 19 |
| Benzovindiflupyr | 0.016 | | | | |
| I.M | 0.016 | 1:4 | 30 | 10 | 20 |
| Bixafen | 0.063 | | | | |
| I.M | 0.016 | 1:63 | 34 | 8 | 26 |
| Boscalid | 1 | | | | |
| I.M | 0.004 | 1:63 | 57 | 22 | 35 |
| Captan | 0.25 | | | | |
| I.M | 0.001 | 1:250 | 61 | 23 | 38 |
| Captan | 0.25 | | | | |
| I.M | 0.00025 | 1:1000 | 60 | 23 | 37 |
| Captan | 0.25 | | | | |
| I.M | 0.016 | 1:4 | 84 | 63 | 21 |
| Carbendazim | 0.063 | | | | |
| I.M | 0.016 | 1:1000 | 46 | 18 | 28 |
| Cyflufenamid | 16 | | | | |
| I.M | 0.016 | 0.08541667 | 59 | 40 | 19 |
| Cyprodinil | 1 | | | | |
| I.M | 0.016 | 1:250 | 34 | 11 | 23 |
| Cyproconazol | 4 | | | | |
| I.M | 0.004 | 1:4000 | 76 | 22 | 54 |
| Cyproconazol | 16 | | | | |
| I.M | 0.016 | 1:16 | 38 | 20 | 18 |
| Difenoconazol | 0.25 | | | | |
| I.M | 0.016 | 1:4 | 35 | 7 | 28 |
| Difenoconazol | 0.063 | | | | |
| I.M | 0.016 | 1:16 | 67 | 49 | 18 |
| Dimoxystrobin | 0.25 | | | | |
| I.M | 0.001 | 1:63 | 41 | 22 | 19 |
| Dithianon | 0.063 | | | | |
| I.M | 0.016 | 1:4 | 32 | 12 | 20 |
| Epoxiconazol | 0.063 | | | | |
| I.M | 0.016 | 1:250 | 77 | 51 | 26 |
| Fenpropidin | 4 | | | | |
| I.M | 0.016 | 1:63 | 56 | 15 | 41 |
| Fenpropidin | 1 | | | | |
| I.M | 0.004 | 1:1000 | 75 | 48 | 27 |
| Fenpropidin | 4 | | | | |
| I.M | 0.016 | 1:16 | 57 | 21 | 36 |
| Fluopyram | 0.25 | | | | |
| I.M | 0.016 | 1:4 | 47 | 9 | 38 |
| Fluopyram | 0.063 | | | | |
| I.M | 0.016 | 1:4 | 33 | 15 | 18 |
| Fluoxastrobin | 0.063 | | | | |
| I.M | 0.016 | 1:63 | 39 | 19 | 20 |
| Fluquinconazol | 1 | | | | |
| I.M | 0.016 | 1:16 | 42 | 9 | 33 |
| Fluquinconazol | 0.25 | | | | |
| I.M | 0.016 | 1:4 | 36 | 17 | 19 |
| Fluxapyroxad | 0.063 | | | | |
| I.M | 0.016 | 1:1 | 38 | 8 | 30 |
| Fluxapyroxad | 0.016 | | | | |
| I.M | 0.004 | 1:250 | 55 | 21 | 34 |
| Folpet | 1 | | | | |
| I.M | 0.016 | 1:16 | 69 | 49 | 20 |
| Kresoxim-methyl | 0.25 | | | | |
| I.M | 0.001 | 1:250 | 63 | 46 | 17 |
| Kresoxim-methyl | 0.25 | | | | |
| I.M | 0.016 | 1:63 | 56 | 29 | 27 |
| Mancozeb | 1 | | | | |
| I.M | 0.016 | 1:16 | 61 | 8 | 53 |
| Mancozeb | 0.25 | | | | |
| I.M | 0.016 | 4:1 | 43 | 8 | 35 |
| Mefentrifluconazol | 0.004 | | | | |
| I.M | 0.016 | 1:4 | 42 | 17 | 25 |
| Metconazol | 0.063 | | | | |
| I.M | 0.016 | 1:1 | 36 | 10 | 26 |
| Metconazol | 0.016 | | | | |
| I.M | 0.016 | 1:250 | 38 | 20 | 18 |
| Metrafenone | 4 | | | | |
| I.M | 0.016 | 1:4 | 35 | 8 | 27 |
| Penthiopyrad | 0.063 | | | | |
| I.M | 0.016 | 1:4 | 55 | 27 | 28 |
| Picoxystrobin | 0.063 | | | | |
| I.M | 0.016 | 1:4 | 43 | 10 | 33 |
| Propiconazol | 0.063 | | | | |
| I.M | 0.016 | 1:4 | 40 | 10 | 30 |
| Prothioconazol | 0.063 | | | | |
| I.M | 0.004 | 1:4 | 100 | 60 | 40 |
| Pyraclostrobin | 0.016 | | | | |
| I.M | 0.004 | 4:1 | 75 | 33 | 42 |
| Pyraclostrobin | 0.001 | | | | |
| I.M | 0.016 | 1:4 | 30 | 9 | 21 |
| Tebuconazol | 0.063 | | | | |
| I.M | 0.016 | 1:1 | 42 | 15 | 27 |
| Trifloxystrobin | 0.016 | | | | |

TABLE II.6
Use examples of synergistic fungicidal activity against
Colletotrichum orbiculare of compound I.E
as component A and components B

| | | | | Calculated | |
|---|---|---|---|---|---|
| Active | | | | efficacy | |
| compound/ | Concen- | | | according | Syner- |
| active mixture | tration | Ratio | Observed | to Colby | gism |
| A + B | (ppm) | A: B | efficacy | (%) | (%) |
| I.E | 16 | — | 52 | | |
| | 4 | — | 11 | | |
| I.E | 1 | — | 4 | | |
| | 0.25 | — | 4 | | |
| I.E | 16 | 16:1 | 84 | 58 | 26 |
| K.1.42 | 1 | | | | |
| I.E | 16 | 16:1 | 82 | 59 | 23 |
| A.3.32 | 1 | | | | |
| I.E | 16 | 16:1 | 89 | 58 | 31 |
| A.2.5 | 1 | | | | |
| I.E | 16 | 16:1 | 84 | 57 | 27 |
| A.3.29 | 1 | | | | |
| I.E | 16 | 1000:1 | 86 | 58 | 28 |
| A.1.25 | 0.016 | | | | |
| I.E | 16 | 1:4 | 87 | 58 | 29 |
| Ametoctradin | 63 | | | | |
| I.E | 16 | 4:1 | 87 | 62 | 25 |
| Benalaxyl | 4 | | | | |
| I.E | 4 | 63:1 | 90 | 45 | 45 |
| Benomyl | 0.063 | | | | |
| I.E | 0.25 | 4:1 | 86 | 41 | 45 |
| Benomyl | 0.063 | | | | |
| I.E | 1 | 1:16 | 86 | 41 | 45 |
| Benomyl | 0.063 | | | | |
| I.E | 16 | 63:1 | 56 | 25 | 31 |
| Captan | 0.25 | | | | |
| I.E | 16 | 250:1 | 86 | 65 | 21 |
| Carbendazim | 0.063 | | | | |
| I.E | 16 | 4:1 | 95 | 62 | 33 |
| Dimethomorph | 4 | | | | |
| I.E | 16 | 16:1 | 78 | 58 | 20 |
| Fluindapyr | 1 | | | | |
| I.E | 4 | 4:1 | 89 | 29 | 60 |
| Folpet | 1 | | | | |
| I.E | 1 | 1:1 | 65 | 24 | 41 |
| Folpet | 1 | | | | |
| I.E | 16 | 16:1 | 90 | 61 | 29 |
| Folpet | 1 | | | | |
| I.E | 16 | 63:1 | 82 | 53 | 29 |
| A.3.24 | 0.25 | | | | |
| I.E | 16 | 16:1 | 73 | 57 | 16 |
| A.3.24 | 1 | | | | |
| I.E | 16 | 63:1 | 73 | 54 | 19 |
| Isoflucypram | 0.25 | | | | |
| I.E | 16 | 16:1 | 93 | 57 | 36 |
| Isoflucypram | 1 | | | | |
| I.E | 16 | 63:1 | 89 | 59 | 30 |
| Tebuconazol | 0.25 | | | | |
| I.E | 16 | 250:1 | 77 | 53 | 24 |
| Tebuconazol | 0.063 | | | | |
| I.E | 16 | 16:1 | 80 | 57 | 23 |
| Triticonazol | 1 | | | | |

TABLE II.7
Use examples of synergistic fungicidal activity against Colletotrichum
orbiculare of compound I.A as component A and components B

| | | | | Calculated | |
|---|---|---|---|---|---|
| Active | | | | efficacy | |
| compound/ | Concen- | | | according | Syner- |
| active mixture | tration | Ratio | Observed | to Colby | gism |
| A + B | (ppm) | A:B | efficacy | (%) | (%) |
| I.A | 0.063 | — | 71 | | |
| | 0.016 | — | 2 | | |
| | 0.004 | — | 2 | | |
| | 0.001 | — | 2 | | |
| | 0.00025 | — | 2 | | |
| I.A | 0.016 | 1:63 | 100 | 4 | 96 |
| Benalaxyl-M | 1 | | | | |
| I.A | 0.001 | 1:63 | 67 | 39 | 28 |
| Benomyl | 0.063 | | | | |
| I.A | 0.00025 | 1:250 | 70 | 40 | 30 |
| Benomyl | 0.063 | | | | |
| I.A | 0.004 | 1:16 | 70 | 39 | 31 |
| Benomyl | 0.063 | | | | |
| I.A | 0.016 | 1:4 | 85 | 61 | 24 |
| Carbendazim | 0.063 | | | | |
| I.A | 0.016 | 1:250 | 60 | 40 | 20 |
| Cyprodinil | 4 | | | | |
| I.A | 0.016 | 1:63 | 55 | 22 | 33 |
| Folpet | 1 | | | | |
| I.A | 0.016 | 16:1 | 52 | 33 | 19 |
| Pyraclostrobin | 0.001 | | | | |
| I.A | 0.016 | 1:1 | 96 | 61 | 35 |
| Pyraclostrobin | 0.016 | | | | |
| I.A | 0.016 | 1:16 | 34 | 16 | 18 |
| Tebuconazol | 0.25 | | | | |

111) Fungicidal Activity Against Leptosphaeria nodorum

The active compounds were formulated separately as a stock solution having a concentration of 10000 ppm in dimethyl sulfoxide. The product sulfur was used as a commercial finished formulation and diluted with water to the stated concentration of the active compound. The stock solutions were mixed according to the ratio, pipetted onto a microtiter plate (MTP) and diluted with water to the stated concentrations. spore suspension of Letosphaeria nodorum in an aqueous biomalt or yeast-bactopeptone-sodiumacetate solution was then added. The plates were placed in a water vapor-saturated chamber at a temperature of 18° C. Using an absorption photometer, the MTPs were measured at 405 nm 7 days after the inoculation. The measured parameters were compared to the growth of the active compound-free control variant (100%) and the fungus-free and active compound-free blank value to determine the relative growth in % of the pathogens in the respective active compounds. These percentages were converted into efficacies.

TABLE III.1
Use examples of fungicidal activity against Leptosphaeria nodorum
of I.H/I.J as component A and components B

| | | Concentration | |
|---|---|---|---|
| Active mixture | Code of | A + B | Efficacy |
| (component A + B) | comnponent B | (ppm) | (ppm) |
| I.H/I.J + component B | A.1.34 | 16 + 63 | 96 |
| I.H/I.J + component B | A.3.29 | 16 + 1 | 79 |
| I.H/I.J + component B | A.2.3 | 16 + 1 | 98 |
| I.H/I.J + component B | K.1.29 | 16 + 16 | 100 |
| I.H/I.J + component B | K.1.30 | 16 + 4 | 88 |
| I.H/I.J + component B | K.1.32 | 16 + 1 | 85 |
| I.H/I.J + component B | K.1.31 | 16 + 4 | 94 |
| I.H/I.J + component B | K.1.54 | 16 + 1 | 58 |
| I.H/I.J + Benomyl | D.1.1 | 16 + 4 | 100 |
| I.H/I.J + Captan | H.3.4 | 16 + 4 | 100 |
| I.H/I.J + Chlorothalonil | H.3.2 | 16 + 1 | 100 |
| I.H/I.J + Cyazofamid | A.2.1 | 16 + 16 | 88 |
| I.H/I.J + Cyproconazol | B.1.4 | 16 + 16 | 99 |
| I.H/I.J + Cyprodinil | E.1.1 | 16 + 4 | 100 |
| I.H/I.J + Difenoconazol | B.1.5 | 16 + 0.25 | 100 |
| I.H/I.J + Diithianon | H.4.9 | 16 + 1 | 100 |
| I.H/I.J + Dimoxystrobin | A.1.4 | 16 + 1 | 93 |
| I.H/I.J + Epoxiconazol | B.1.8. | 16 + 0.25 | 85 |
| I.H/I.J + Fenpropidin | B.2.6 | 16 + 4 | 64 |
| I.H/I.J + Florylpicoxamid | A.2.6 | 16 + 1 | 99 |
| I.H/I.J + Fluquinconazol | B.1.10 | 16 + 1 | 97 |
| I.H/I.J + Kresoxim-methyl | A.1.9 | 16 + 1 | 96 |
| I.H/I.J + Metconazol | B.1.17 | 16 + 0.063 | 58 |
| I.H/I.J + Metominostrobin | A.1.11 | 16 + 4 | 100 |
| I.H/I.J + Picoxystrobin | A.1.13 | 16 + 0.25 | 95 |
| I.H/I.J + Propiconazol | B.1.22 | 16 + 0.25 | 63 |
| I.H/I.J + Pydiflumetofen | A.3.17 | 16 + 16 | 99 |
| I.H/I.J + Pyraclostrobin | A.1.14 | 16 + 0.016 | 95 |
| I.H/I.J + Sulfur | H.1.7 | 16 + 63 | 93 |

TABLE III.1
Use examples of fungicidal activity against Leptosphaeria nodorum
of I. F as component A and components B

| | | Concentration | |
|---|---|---|---|
| Active mixture | Code of | A + B | Efficacy |
| (component A + B) | component B | (ppm) | (ppm) |
| I.F + component B | A.1.34 | 4 + 63 | 100 |
| I.F + component B | A.3.29 | 4 + 1 | 80 |
| I.F + component B | A.2.3 | 4 + 1 | 100 |
| I.F + component B | A.2.5 | 4 + 1 | 94 |
| I.F + component B | K.1.54 | 4 + 1 | 66 |
| I.F + Azoxystrobin | A.1.1 | 4 + 0.063 | 55 |
| I.F + Benomyl | D.1.1 | 4 + 4 | 99 |
| I.F + Captan | H.3.4 | 4 + 4 | 100 |
| I.F + Chlorothalonil | H.3.2 | 4 + 1 | 94 |
| I.F + Cyazofamid | A.2.1 | 4 + 16 | 63 |
| I.F + Cyproconazol | B.1.4 | 4 + 16 | 100 |
| I.F + Cyprodinil | E.1.1 | 4 + 4 | 100 |
| I.F + Difenoconazol | B.1.5 | 4 + 0.25 | 96 |
| I.F + Dimoxystrobin | A.1.4 | 4 + 1 | 86 |
| I.F + Dithianon | H.4.9 | 4 + 1 | 100 |
| I.F + Epoxiconazol | B.1.8. | 4 + 0.25 | 81 |
| I.F + Fenpropidin | B.2.6 | 4 + 4 | 71 |
| I.F + Fluquinconazol | B.1.10 | 4 + 1 | 98 |
| I.F + Mefentrifluconazol | B.1.38 | 4 + 0.016 | 54 |
| I.F + Metconazol | B.1.17 | 4 + 0.063 | 67 |
| I.F + Picoxystrobin | A.1.13 | 4 + 0.25 | 91 |
| I.F + Propiconazol | B.1.22 | 4 + 0.25 | 69 |
| I.F + Pyraclostrobin | A.1.14 | 4 + 0.016 | 78 |
| I.F + Tebuconazol | B.1.25 | 4 + 0.25 | 56 |

TABLE III.3
Use examples of fungicidal activity against Leptosphaeria nodorum
of I.B/I.C as component A and components B

| | | Concentration | |
|---|---|---|---|
| Active mixture | Code of | A + B | Efficacy |
| (component A + B) | component B | (ppm) | (ppm) |
| I.B/I.C + component B | A.1.34 | 1 + 63 | 100 |
| I.B/I.C + component B | A.3.29 | 1 + 1 | 80 |
| I.B/I.C + component B | A.2.3 | 1 + 1 | 100 |
| I.B/I.C + component B | A.2.5 | 1 + 1 | 99 |
| I.B/I.C + component B | K.1.29 | 1 + 16 | 100 |
| I.B/I.C + component B | K.1.30 | 1 + 4 | 86 |
| I.B/I.C + component B | K.1.32 | 1 + 1 | 82 |
| I.B/I.C + component B | K.1.31 | 1 + 4 | 91 |
| I.B/I.C + component B | K.1.54 | 1 + 1 | 62 |
| I.B/I.C + Benomyl | D.1.1 | 1 + 4 | 99 |
| I.B/I.C + Captan | H.3.4 | 1 + 4 | 100 |
| I.B/I.C + Chlorothalonil | H.3.2 | 1 + 1 | 96 |
| I.B/I.C + Cyazofamid | A.2.1 | 1 + 16 | 65 |
| I.B/I.C + Cyproconazol | B.1.4 | 1 + 16 | 100 |
| I.B/I.C + Cyprodinil | E.1.1 | 1 + 4 | 100 |
| I.B/I.C + Difenoconazol | B.1.5 | 1 + 0.25 | 99 |
| I.B/I.C + Dimoxystrobin | A.1.4 | 1 + 1 | 97 |
| I.B/I.C + Dithianon | H.4.9 | 1 + 1 | 100 |
| I.B/I.C + Epoxiconazol | B.1.8. | 1 + 0.25 | 87 |
| I.B/I.C + Fenpropidin | B.2.6 | 1 + 4 | 69 |
| I.B/I.C + Florylpicoxamid | A.2.6 | 1 + 1 | 100 |
| I.B/I.C + Fludioxonil | F.1.5 | 1 + 0.25 | 59 |
| I.B/I.C + Fluoxastrobin | A.1.8 | 1 + 0.25 | 50 |
| I.B/I.C + Fluquinconazol | B.1.10 | 1 + 1 | 99 |
| I.B/I.C + Kresoxim-methyl | A.1.9 | 1 + 1 | 100 |
| I.B/I.C + Metconazol | B.1.17 | 1 + 0.063 | 63 |
| I.B/I.C + Metominostrobin | A.1.11 | 1 + 4 | 100 |
| I.B/I.C + Picoxystrobin | A.1.13 | 1 + 0.25 | 96 |
| I.B/I.C + Propiconazol | B.1.22 | 1 + 0.25 | 64 |
| I.B/I.C + Pydiflumetofen | A.3.17 | 1 + 16 | 100 |
| I.B/I.C + Pyraclostrobin | A.1.14 | 1 + 0.016 | 81 |
| I.B/I.C + Sulfur | H.1.7 | 1 + 63 | 100 |

TABLE III.4
Use examples fungicidal activity against Leptosphaeria nodorum
of selected components B

| Code of | Common name | Concentration | Observed |
|---|---|---|---|
| component B | component B | (ppm) | efficacy |
| K.1.32 | | 0.25 | 40 |
| A.1.34 | | 0.063 | 45 |
| A.1.1 | Azoxystrobin | 0.063 | 40 |
| D.1.1 | Benomyl | 1 | 15 |
| H.3.4 | Captan | 1 | 44 |
| | | 0.25 | 13 |
| H.3.2 | Chlorothalonil | 1 | 72 |
| A.2.1 | Cyazofamid | 0.25 | 6 |
| E.1.1 | Cyprodinil | 0.004 | 71 |
| B.1.5 | Difenoconazol | 0.063 | 41 |
| G.3.1 | Dimethomorph | 4 | 41 |
| B.2.6 | Fenpropidin | 1 | 38 |
| A.1.8 | Fluoxastrobin | 0.25 | 38 |
| H.3.5 | Folpet | 1 | 40 |
| B.1.38 | Mefentrifluconazol | 0.016 | 73 |
| | | 0.004 | 53 |
| A.1.11 | Metominostrobin | 1 | 43 |
| A.1.13 | Picoxystrobin | 0.063 | 42 |
| A.1.14 | Pyraclostrobin | 0.004 | 59 |
| | | 0.00025 | 22 |
| H.1.7 | Sulfur | 4 | 30 |

TABLE III.5
Use examples of synergistic fungicidal activity against Leptosphaeria
nodorum of compound I.M as component A and components B

| | | | | Calculated | |
|---|---|---|---|---|---|
| Active | | | | efficacy | |
| compound/ | Concen- | | | according | Syner- |
| active mixture | tration | Ratio | Observed | to Colby | gism |
| A + B | (ppm) | A:B | efficacy | (%) | (%) |
| I.M | 4 | — | 52 | | |
| | 1 | — | 18 | | |
| | 0.25 | — | 7 | | |
| | 0.016 | — | 5 | | |
| | 0.004 | — | 6 | | |
| I.M | 0.016 | 1:4 | 82 | 48 | 34 |
| A.1.34 | 0.063 | | | | |
| I.M | 4 | 63:1 | 91 | 71 | 20 |
| Azoxystrobin | 0.063 | | | | |
| I.M | 4 | 4:1 | 86 | 66 | 20 |
| Benomyl | 1 | | | | |
| I.M | 4 | 16:1 | 100 | 73 | 27 |
| Captan | 0.25 | | | | |
| I.M | 1 | 1:1 | 100 | 54 | 46 |
| Captan | 1 | | | | |
| I.M | 0.25 | 1:4 | 100 | 48 | 52 |
| Captan | 1 | | | | |
| I.M | 1 | 1:1 | 98 | 77 | 21 |
| Chlorothalonil | 1 | | | | |
| I.M | 1 | 4:1 | 100 | 77 | 23 |
| Cyazofamid | 0.25 | | | | |
| I.M | 0.004 | 1:1 | 98 | 73 | 25 |
| Cyprodinil | 0.004 | | | | |
| I.M | 4 | 16:1 | 89 | 70 | 19 |
| Fluoxastrobin | 0.25 | | | | |
| I.M | 1 | 1:1 | 78 | 51 | 27 |
| Folpet | 1 | | | | |
| I.M | 4 | 63:1 | 93 | 72 | 21 |
| Picoxystrobin | 0.063 | | | | |
| I.M | 1 | 250:1 | 100 | 66 | 34 |
| Pyraclostrobin | 0.004 | | | | |
| I.M | 1 | 1:4 | 85 | 42 | 43 |
| Sulfur | 4 | | | | |
| I.M | 0.25 | 1:16 | 66 | 35 | 31 |
| Sulfur | 4 | | | | |

TABLE III.6
Use examples of synergistic fungicidal activity against Leptosphaeria nodorum
of compound I.E as component A and components B

| | | | | Calculated | |
|---|---|---|---|---|---|
| Active | | | | efficacy | |
| compound/ | Concen- | | | according | Syner- |
| active mixture | tration | Ratio | Observed | to Colby | gism |
| A + B | (ppm) | A:B | efficacy | (%) | (%) |
| I.E | 63 | — | 22 | | |
| | 16 | — | 18 | | |
| I.E | 0.25 | 4:1 | 94 | 48 | 46 |
| A.1.34 | 0.063 | | | | |
| I.E | 63 | 250:1 | 95 | 56 | 39 |
| Captan | 0.25 | | | | |
| I.E | 63 | 1000:1 | 75 | 54 | 21 |
| Difenoconazol | 0.063 | | | | |
| I.E | 63 | 4000:1 | 97 | 79 | 18 |
| Mefentrifluconazol | 0.016 | | | | |
| I.E | 16 | 1000:1 | 100 | 79 | 21 |
| Mefentrifluconazol | 0.016 | | | | |
| I.E | 63 | 16000:1 | 94 | 63 | 31 |
| Mefentrifluconazol | 0.004 | | | | |
| I.E | 63 | 63:1 | 78 | 56 | 22 |
| Metominostrobin | 1 | | | | |
| I.E | 63 | 16000:1 | 100 | 68 | 32 |
| Pyraclostrobin | 0.004 | | | | |

TABLE III.7
Use examples of synergistic fungicidal activity against Leptosphaeria nodorum
of compound I.A as component A and components B

| | | | | Calculated | |
|---|---|---|---|---|---|
| Active | | | | efficacy | |
| compound/ | Concen- | | | according | Syner- |
| active mixture | tration | Ratio | Observed | to Colby | gism |
| A + B | (ppm) | A:B | efficacy | (%) | (%) |
| I.A | 16 | — | 27 | | |
| | 4 | — | 14 | | |
| | 1 | — | 6 | | |
| | 0.063 | — | 3 | | |
| I.A | 16 | 63:1 | 77 | 56 | 21 |
| K.1.32 | 0.25 | | | | |
| I.A | 0.063 | 1:1 | 73 | 47 | 26 |
| A.1.34 | 0.063 | | | | |
| I.A | 16 | 250:1 | 80 | 56 | 24 |
| Azoxystrobin | 0.063 | | | | |
| I.A | 16 | 16:1 | 75 | 49 | 26 |
| Benomyl | 1 | | | | |
| I.A | 1 | 1:1 | 97 | 47 | 50 |
| Captan | 1 | | | | |
| I.A | 16 | 16:1 | 100 | 59 | 41 |
| Captan | 1 | | | | |
| I.A | 16 | 16:1 | 100 | 79 | 21 |
| Chlorothalonil | 1 | | | | |
| I.A | 4 | 4:1 | 98 | 76 | 22 |
| Chlorothalonil | 1 | | | | |
| I.A | 16 | 250:1 | 79 | 57 | 22 |
| Difenoconazol | 0.063 | | | | |
| I.A | 4 | 1:1 | 100 | 50 | 50 |
| Dimethomorph | 4 | | | | |
| I.A | 16 | 16:1 | 73 | 55 | 18 |
| Fenpropidin | 1 | | | | |
| I.A | 16 | 63:1 | 85 | 55 | 30 |
| Fluoxastrobin | 0.25 | | | | |
| I.A | 1 | 1:1 | 70 | 44 | 26 |
| Folpet | 1 | | | | |
| I.A | 4 | 4:1 | 68 | 49 | 19 |
| Folpet | 1 | | | | |
| I.A | 4 | 250:1 | 100 | 77 | 23 |
| Mefentrifluconazol | 0.016 | | | | |
| I.A | 16 | 1000:1 | 100 | 80 | 20 |
| Mefentrifluconazol | 0.016 | | | | |
| I.A | 16 | 4000:1 | 95 | 66 | 29 |
| Mefentrifluconazol | 0.004 | | | | |
| I.A | 16 | 250:1 | 82 | 58 | 24 |
| Picoxystrobin | 0.063 | | | | |
| I.A | 0.25 | 1000:1 | 67 | 24 | 43 |
| Pyraclostrobin | 0.00025 | | | | |
| I.A | 0.063 | 250:1 | 68 | 24 | 44 |
| Pyraclostrobin | 0.00025 | | | | |
| I.A | 16 | 400:1 | 100 | 70 | 30 |
| Pyraclostrobin | 0.004 | | | | |
| I.A | 1 | 1:4 | 52 | 34 | 18 |
| Sulfur | 4 | | | | |

IV) Fungicidal Activity Against Pyricularia oryzae

The active compounds were formulated separately as a stock solution having a concentration of 10000 ppm in dimethyl sulfoxide. The stock solutions were mixed according to the ratio, pipetted onto a micro titer plate (MTP) and diluted with water to the stated concentrations. A spore suspension of Pyricularia oryzae in an aqueous biomalt or yeast-bactopeptone-sodiumacetate solution was then added. The plates were placed in a water vapor-saturated chamber at a temperature of 18° C. Using an absorption photometer, the MTPs were measured at 405 nm 7 days after the inoculation. The measured parameters were compared to the growth of the active compound-free control variant (100%) and the fungus-free and active compound-free blank value to determine the relative growth in % of the pathogens in the respective active compounds. These percentages were converted into efficacies.

TABLE IV.1
Use examples of fungicidal activity against Pyricularia oryzae
of I.H/I.J as component A and components B

| | | Concentration | |
|---|---|---|---|
| Active mixture | Code of | A + B | Efficacy |
| (component A + B) | component B | (ppm) | (ppm) |
| I.H/I.J + component B | A.1.34 | 16 + 63 | 95 |
| I.H/I.J + component B | A.1.25 | 16 + 0.016 | 64 |
| I.H/I.J + component B | A.2.3 | 16 + 1 | 98 |
| I.H/I.J + component B | A.2.5 | 16 + 1 | 97 |
| I.H/I.J + component B | K.1.29 | 16 + 16 | 99 |
| I.H/I.J + component B | A.3.34 | 16 + 1 | 99 |
| I.H/I.J + component B | K.1.30 | 16 + 4 | 99 |
| I.H/I.J + component B | K.1.32 | 16 + 1 | 100 |
| I.H/I.J + component B | K.1.31 | 16 + 4 | 100 |
| I.H/I.J + Azoxystrobin | A.1.1 | 16 + 0.063 | 89 |
| I.H/I.J + Benomyl | D.1.1 | 16 + 4 | 100 |
| I.H/I.J + Benzovindiflupyr | A.3.2 | 16 + 0.063 | 98 |
| I.H/I.J + Captan | H.3.4 | 16 + 4 | 100 |
| I.H/I.J + Carbendazim | D.1.2 | 16 + 0.25 | 100 |
| I.H/I.J + Chlorothalonil | H.3.2 | 16 + 1 | 99 |
| I.H/I.J + Cyazofamid | A.2.1 | 16 + 16 | 96 |
| I.H/I.J + Cyflufenamid | K.1.3 | 16 + 63 | 69 |
| I.H/I.J + Cyproconazol | B.1.4 | 16 + 16 | 99 |
| I.H/I.J + Cyprodinil | E.1.1 | 16 + 4 | 80 |
| I.H/I.J + Dimoxystrobin | A.1.4 | 16 + 1 | 94 |
| I.H/I.J + Dithianon | H.4.9 | 16 + 1 | 98 |
| I.H/I.J + Florylpicoxamid | A.2.6 | 16 + 1 | 92 |
| I.H/I.J + Fludioxonil | F.1.5 | 16 + 0.25 | 68 |
| I.H/I.J + Fluoxastrobin | A.1.8 | 16 + 0.25 | 81 |
| I.H/I.J + component B | A.3.24 | 16 + 1 | 80 |
| I.H/I.J + Kresoxim-methyl | A.1.9 | 16 + 1 | 61 |
| I.H/I.J + Metominostrobin | A.1.11 | 16 + 4 | 88 |
| I.H/I.J + Picoxystrobin | A.1.13 | 16 + 0.25 | 93 |
| I.H/I.J + Pydiflumetofen | A.3.17 | 16 + 16 | 97 |
| I.H/I.J + Pyraclostrobin | A.1.14 | 16 + 0.016 | 96 |
| I.H/I.J + Sulfur | H.1.7 | 16 + 63 | 77 |
| I.H/I.J + Trifloxystrobin | A.1.17 | 16 + 0.063 | 97 |

TABLE IV.2
Use examples of fungicidal activity against Pyricularia oryzae
of I.F as component A and components B

| | | Concentration | |
|---|---|---|---|
| Active mixture | Code of | A + B | Efficacy |
| (component A + B) | component B | (ppm) | (ppm) |
| I.F + component B | A.1.34 | 4 + 63 | 94 |
| I.F + component B | A.1.25 | 4 + 0.016 | 57 |
| I.F + component B | A.2.3 | 4 + 1 | 98 |
| I.F + component B | A.2.5 | 4 + 1 | 97 |
| I.F + component B | A.3.34 | 4 + 1 | 100 |
| I.F + component B | A.3.32 | 4 + 1 | 60 |
| I.F + Azoxystrobin | A.1.1 | 4 + 0.063 | 89 |
| I.F + Benomyl | D.1.1 | 4 + 4 | 100 |
| I.F + Benzovindiflupyr | A.3.2 | 4 + 0.063 | 99 |
| I.F + Captan | H.3.4 | 4 + 4 | 100 |
| I.F + Carbendazim | D.1.2 | 4 + 0.25 | 100 |
| I.F + Chlorothalonil | H.3.2 | 4 + 1 | 100 |
| I.F + Cyazofamid | A.2.1 | 4 + 16 | 100 |
| I.F + Cyprodinil | E.1.1 | 4 + 4 | 80 |
| I.F + Dithianon | H.4.9 | 4 + 1 | 100 |
| I.F + Fludioxonil | F.1.5 | 4 + 0.25 | 64 |
| I.F + Fluindapyr | A.3.28 | 4 + 1 | 59 |
| I.F + component B | A.3.24 | 4 + 1 | 84 |
| I.F + Picoxystrobin | A.1.13 | 4 + 0.25 | 97 |
| I.F + Pyraclostrobin | A.1.14 | 4 + 0.016 | 97 |
| I.F + Trifloxystrobin | A.1.17 | 4 + 0.063 | 98 |

TABLE IV.3
Use examples of fungicidal activity against Pyricularia oryzae
of I.B/I.C as component A and components B

| | | Concentration | |
|---|---|---|---|
| Active mixture | Code of | A + B | Efficacy |
| (component A + B) | component B | (ppm) | (ppm) |
| I.B/I.C + component B | A.1.34 | 1 + 63 | 98 |
| I.B/I.C + component B | A.1.25 | 1 + 0.016 | 79 |
| I.B/I.C + component B | A.2.3 | 1 + 1 | 99 |
| I.B/I.C + component B | A.2.5 | 1 + 1 | 98 |
| I.B/I.C + component B | K.1.29 | 1 + 16 | 100 |
| I.B/I.C + component B | K.1.29 | 1 + 1 | 100 |
| I.B/I.C + component B | A.3.34 | 1 + 1 | 57 |
| I.B/I.C + component B | A.3.32 | 1 + 4 | 100 |
| I.B/I.C + component B | K.1.30 | 1 + 1 | 100 |
| I.B/I.C + component B | K.1.32 | 1 + 4 | 100 |
| I.B/I.C + Azoxystrobin | K.1.31 | 1 + 0.063 | 96 |
| I.B/I.C + Benomyl | D.1.1 | 1 + 4 | 100 |
| I.B/I.C + Benzovindiflupyr | A.3.2 | 1 + 0.063 | 100 |
| I.B/I.C + Captan | H.3.4 | 1 + 4 | 100 |
| I.B/I.C + Carbendazim | D.1.2 | 1 + 0.25 | 100 |
| I.B/I.C + Chlorothalonil | H.3.2 | 1 + 1 | 100 |
| I.B/I.C + Cyazofamid | A.2.1 | 1 + 16 | 100 |
| I.B/I.C + Cyflufenamid | K.1.3 | 1 + 63 | 100 |
| I.B/I.C + Cyproconazol | B.1.4 | 1 + 16 | 99 |
| I.B/I.C + Cyprodinil | E.1.1 | 1 + 4 | 86 |
| I.B/I.C + Dimoxystrobin | A.1.4 | 1 + 1 | 99 |
| I.B/I.C + Dithianon | H.4.9 | 1 + 1 | 100 |
| I.B/I.C + Florylpicoxamid | A.2.6 | 1 + 1 | 94 |
| I.B/I.C + Fludioxonil | F.1.5 | 1 + 0.25 | 84 |
| I.B/I.C + Fluindapyr | A.3.28 | 1 + 1 | 54 |
| I.B/I.C + Fluoxastrobin | A.1.8 | 1 + 0.25 | 94 |
| I.B/I.C + component B | A.3.24 | 1 + 1 | 90 |
| I.B/I.C + Kresoxim-methyl | A.1.9 | 1 + 1 | 65 |
| I.B/I.C + Metominostrobin | A.1.11 | 1 + 4 | 88 |
| I.B/I.C + Picoxystrobin | A.1.13 | 1 + 0.25 | 99 |
| I.B/I.C + Pydiflumetofen | A.3.17 | 1 + 16 | 100 |
| I.B/I.C + Pyraclostrobin | A.1.14 | 1 + 0.016 | 99 |
| I.B/I.C + Sulfur | H.1.7 | 1 + 63 | 100 |
| I.B/I.C + Trifloxystrobin | A.1.17 | 1 + 0.063 | 99 |

TABLE IV.4
Use examples fungicidal activity against
Pyricularia oryzae of selected components B

| Code of | Common name | Concentration | Observed |
|---|---|---|---|
| component B | component B | (ppm) | efficacy |
| K.1.42 | | 1 | 9 |
| | | 0.25 | 5 |
| A.3.29 | | 1 | 14 |
| | | 0.25 | 2 |
| A.2.3 | | 0.063 | 54 |
| A.2.5 | | 0.063 | 44 |
| A.3.34 | | 0.25 | 40 |
| A.3.32 | | 0.25 | 34 |
| K.1.54 | | 1 | 19 |
| | | 0.25 | 4 |
| A.1.25 | | 0.016 | 30 |
| | | 0.004 | 4 |
| A.4.11 | Ametoctradin | 63 | 13 |
| | | 16 | 8 |
| A.1.1 | Azoxystrobin | 0.016 | 28 |
| C.1.1 | Benalaxyl | 4 | 8 |
| | | 1 | 2 |
| C.1.2 | Benalaxyl-M | 4 | 12 |
| | | 1 | 3 |
| D.1.1 | Benomyl | 0.25 | 19 |
| A.3.2 | Benzovindiflupyr | 0.016 | 70 |
| A.3.4 | Boscalid | 4 | 5 |
| H.3.4 | Captan | 1 | 53 |
| | | 0.25 | 13 |
| D.1.2 | Carbendazim | 0.063 | 3 |
| A.2.1 | Cyazofamid | 4 | 13 |
| B.1.4 | Cyproconazol | 0.25 | 2 |
| E.1.1 | Cyprodinil | 1 | 56 |
| G.3.1 | Dimethomorph | 4 | 8 |
| | | 1 | 4 |
| A.1.4 | Dimoxystrobin | 0.016 | 34 |
| H.4.9 | Dithianon | 0.25 | 78 |
| B.1.8. | Epoxiconazol | 0.25 | 20 |
| B.2.4 | Fenpropimorph | 1 | 8 |
| F.1.5 | Fludioxonil | 0.25 | 61 |
| | | 0.063 | 45 |
| A.1.8 | Fluoxastrobin | 0.063 | 58 |
| H.3.5 | Folpet | 1 | 14 |
| A.3.24 | | 0.25 | 5 |
| A.3.29 | Isoflucypram | 1 | 11 |
| | | 0.25 | 5 |
| A.1.9 | Kresoximmethyl | 1 | 58 |
| | | 0.25 | 20 |
| A.1.11 | Metominostrobin | 1 | 59 |
| D.2.6 | Metrafenon | 16 | 21 |
| | | 4 | 5 |
| A.3.16 | Penthiopyrad | 0.25 | 14 |
| A.1.13 | Picoxystrobin | 0.063 | 63 |
| B.1.23 | Prothioconazol | 0.25 | 11 |
| A.1.14 | Pyraclostrobin | 0.001 | 46 |
| B.1.25 | Tebuconazol | 0.25 | 20 |
| | | 0.0663 | 4 |
| A.1.17 | Trifloxystrobin | 0.016 | 27 |

TABLE IV.5
Use examples of synergistic fungicidal activity against Pyricularia oryzae
of compound I.M as component A and components B

| | | | | Calculated | |
|---|---|---|---|---|---|
| Active | | | | efficacy | |
| compound/ | Concen- | | | according | Syner- |
| active mixture | tration | Ratio | Observed | to Colby | gism |
| A + B | (ppm) | A:B | efficacy | (%) | (%) |
| I.M | 4 | — | 32 | | |
| | 1 | — | 9 | | |
| | 0.25 | — | 6 | | |
| | 0.063 | — | 5 | | |
| I.M | 4 | 4:1 | 72 | 41 | 31 |
| A.3.29 | 1 | | | | |
| I.M | 4 | 16:1 | 60 | 33 | 27 |
| A.3.29 | 0.25 | | | | |
| I.M | 0.25 | 4:1 | 78 | 56 | 22 |
| A.2.3 | 0.063 | | | | |
| I.M | 1 | 16:1 | 77 | 58 | 19 |
| A.2.3 | 0.063 | | | | |
| I.M | 1 | 4:1 | 70 | 45 | 25 |
| A.2.5 | 0.25 | | | | |
| I.M | 4 | 16:1 | 88 | 59 | 29 |
| A.2.5 | 0.25 | | | | |
| I.M | 4 | 16:1 | 59 | 33 | 26 |
| A.3.32 | 0.25 | | | | |
| I.M | 4 | 4:1 | 75 | 45 | 30 |
| K.1.54 | 1 | | | | |
| I.M | 4 | 16:1 | 65 | 34 | 31 |
| K.1.54 | 0.25 | | | | |
| I.M | 4 | 250:1 | 88 | 52 | 36 |
| A.1.25 | 0.016 | | | | |
| I.M | 4 | 1000:1 | 80 | 35 | 45 |
| A.1.25 | 0.004 | | | | |
| I.M | 4 | 250:1 | 84 | 51 | 33 |
| Azoxystrobin | 0.016 | | | | |
| I.M | 4 | 1:1 | 66 | 38 | 28 |
| Benalaxyl | 4 | | | | |
| I.M | 4 | 4:1 | 65 | 33 | 32 |
| Benalaxyl | 1 | | | | |
| I.M | 4 | 1:1 | 81 | 40 | 41 |
| Benalaxyl-M | 4 | | | | |
| I.M | 4 | 4:1 | 71 | 34 | 37 |
| Benalaxyl-M | 1 | | | | |
| I.M | 0.25 | 1:1 | 97 | 23 | 74 |
| Benomyl | 0.25 | | | | |
| I.M | 0.063 | 1:4 | 97 | 23 | 74 |
| Benomyl | 0.25 | | | | |
| I.M | 1 | 4:1 | 94 | 26 | 68 |
| Benomyl | 0.25 | | | | |
| I.M | 4 | 1:1 | 60 | 36 | 24 |
| Boscalid | 4 | | | | |
| I.M | 1 | 1:1 | 100 | 57 | 43 |
| Captan | 1 | | | | |
| I.M | 0.25 | 1:4 | 100 | 55 | 45 |
| Captan | 1 | | | | |
| I.M | 4 | 4:1 | 100 | 68 | 32 |
| Captan | 1 | | | | |
| I.M | 4 | 63:1 | 62 | 34 | 28 |
| Carbendazim | 0.063 | | | | |
| I.M | 1 | 1:4 | 76 | 21 | 55 |
| Cyazofamid | 4 | | | | |
| I.M | 4 | 1:1 | 100 | 41 | 59 |
| Cyazofamid | 4 | | | | |
| I.M | 4 | 4:1 | 90 | 70 | 20 |
| Cyprodinil | 1 | | | | |
| I.M | 4 | 1:1 | 59 | 37 | 22 |
| Dimethomorph | 4 | | | | |
| I.M | 4 | 4:1 | 60 | 34 | 26 |
| Dimethomorph | 1 | | | | |
| I.M | 4 | 16:1 | 100 | 80 | 20 |
| Dithianon | 0.25 | | | | |
| I.M | 0.25 | 1:1 | 98 | 79 | 19 |
| Dithianon | 0.25 | | | | |
| I.M | 4 | 16:1 | 100 | 74 | 26 |
| Fludioxonil | 0.25 | | | | |
| I.M | 1 | 4:1 | 94 | 65 | 29 |
| Fludioxonil | 0.25 | | | | |
| I.M | 4 | 63:1 | 100 | 63 | 37 |
| Fludioxonil | 0.063 | | | | |
| I.M | 4 | 4:1 | 76 | 54 | 22 |
| Fluindapyr | 1 | | | | |
| I.M | 4 | 63:1 | 96 | 71 | 25 |
| Fluoxastrobin | 0.063 | | | | |
| I.M | 1 | 1:1 | 49 | 22 | 27 |
| Folpet | 1 | | | | |
| I.M | 4 | 16:1 | 59 | 32 | 27 |
| Fluindapyr | 0.25 | | | | |
| I.M | 4 | 16:1 | 62 | 35 | 27 |
| A.3.24 | 0.25 | | | | |
| I.M | 4 | 4:1 | 76 | 39 | 37 |
| Isoflucypram | 1 | | | | |
| I.M | 4 | 16:1 | 65 | 35 | 30 |
| Isoflucypram | 0.25 | | | | |
| I.M | 4 | 16:1 | 97 | 46 | 51 |
| Kresoximmethyl | 0.25 | | | | |
| I.M | 4 | 4:1 | 100 | 72 | 28 |
| Kresoximmethyl | 1 | | | | |
| I.M | 1 | 1:1 | 88 | 62 | 26 |
| Kresoximmethyl | 1 | | | | |
| I.M | 4 | 1:4 | 70 | 46 | 24 |
| Metrafenon | 16 | | | | |
| I.M | 4 | 1:1 | 62 | 36 | 26 |
| Metrafenon | 4 | | | | |
| I.M | 1 | 1:16 | 99 | 28 | 71 |
| Metrafenon | 16 | | | | |
| I.M | 4 | 63:1 | 99 | 75 | 24 |
| Picoxystrobin | 0.063 | | | | |
| I.M | 0.25 | 250:1 | 96 | 49 | 47 |
| Pyraclostrobin | 0.001 | | | | |
| I.M | 0.063 | 63:1 | 90 | 49 | 41 |
| Pyraclostrobin | 0.001 | | | | |
| I.M | 1 | 1000:1 | 97 | 51 | 46 |
| Pyraclostrobin | 0.001 | | | | |
| I.M | 4 | 16:1 | 77 | 45 | 32 |
| Tebuconazol | 0.25 | | | | |
| I.M | 4 | 63:1 | 64 | 35 | 29 |
| Tebuconazol | 0.063 | | | | |
| I.M | 1 | 63:1 | 57 | 34 | 23 |
| Trifloxystrobin | 0.016 | | | | |
| I.M | 4 | 250:1 | 98 | 51 | 47 |
| Trifloxystrobin | 0.016 | | | | |

TABLE IV.6
Use examples of synergistic fungicidal activity against Pyricularia oryzae
of compound I.E as component A and components B

| | | | | Calculated | |
|---|---|---|---|---|---|
| Active | | | | efficacy | |
| compound/ | Concen- | | | according | Syner- |
| active mixture | tration | Ratio | Observed | to Colby | gism |
| A + B | (ppm) | A:B | efficacy | (%) | (%) |
| I.E | 63 | — | 9 | | |
| | 16 | — | 6 | | |
| | 4 | — | 5 | | |
| | 1 | — | 5 | | |
| I.E | 16 | 250:1 | 82 | 48 | 34 |
| A.2.5 | 0.063 | | | | |
| I.E | 63 | 250:1 | 84 | 45 | 39 |
| A.2.5 | 0.25 | | | | |
| I.E | 63 | 4000:1 | 73 | 37 | 36 |
| A.1.25 | 0.016 | | | | |
| I.E | 63 | 4000:1 | 80 | 35 | 45 |
| Azoxystrobin | 0.016 | | | | |
| I.E | 4 | 16:1 | 74 | 23 | 51 |
| Benomyl | 0.25 | | | | |
| I.E | 1 | 4:1 | 89 | 23 | 66 |
| Benomyl | 0.25 | | | | |
| I.E | 16 | 63:1 | 91 | 23 | 68 |
| Benomyl | 0.25 | | | | |
| I.E | 16 | 16:1 | 89 | 56 | 33 |
| Captan | 1 | | | | |
| I.E | 4 | 4:1 | 92 | 55 | 37 |
| Captan | 1 | | | | |
| I.E | 63 | 63:1 | 100 | 57 | 43 |
| Captan | 1 | | | | |
| I.E | 63 | 1000:1 | 75 | 50 | 25 |
| Fludioxonil | 0.063 | | | | |
| I.E | 63 | 1000:1 | 92 | 62 | 30 |
| Fluoxastrobin | 0.063 | | | | |
| I.E | 63 | 63:1 | 90 | 62 | 28 |
| Kresoximmethyl | 1 | | | | |
| I.E | 63 | 250:1 | 79 | 28 | 51 |
| Kresoximmethyl | 0.25 | | | | |
| I.E | 63 | 1000:1 | 86 | 66 | 20 |
| Picoxystrobin | 0.063 | | | | |
| I.E | 4 | 4000:1 | 93 | 49 | 44 |
| Pyraclostrobin | 0.001 | | | | |
| I.E | 16 | 16000:1 | 96 | 49 | 47 |
| Pyraclostrobin | 0.001 | | | | |
| I.E | 1 | 1000:1 | 93 | 49 | 44 |
| Pyraclostrobin | 0.001 | | | | |
| I.E | 63 | 4000:1 | 88 | 34 | 54 |
| Trifloxystrobin | 0.016 | | | | |

TABLE IV.7
Use examples of synergistic fungicidal activity against Pyricularia oryzae
of compound I.A as component A and components B

| | | | | Calculated | |
|---|---|---|---|---|---|
| Active | | | | efficacy | |
| compound/ | Concen- | | | according | Syner- |
| active mixture | tration | Ratio | Observed | to Colby | gism |
| A + B | (ppm) | A:B | efficacy | (%) | (%) |
| I.A | 16 | — | 38 | | |
| | 4 | — | 12 | | |
| | 1 | — | 6 | | |
| | 0.25 | — | 2 | | |
| I.A | 1 | 16:1 | 66 | 48 | 18 |
| A.2.5 | 0.063 | | | | |
| I.A | 4 | 16:1 | 73 | 47 | 26 |
| A.2.5 | 0.25 | | | | |
| I.A | 1 | 4:1 | 66 | 43 | 23 |
| A.2.5 | 0.25 | | | | |
| I.A | 16 | 63:1 | 95 | 62 | 33 |
| A.2.5 | 0.25 | | | | |
| I.A | 16 | 1000:1 | 77 | 56 | 21 |
| A.1.25 | 0.016 | | | | |
| I.A | 4 | 250:1 | 71 | 36 | 35 |
| Azoxystrobin | 0.016 | | | | |
| I.A | 1 | 63:1 | 55 | 32 | 23 |
| Azoxystrobin | 0.016 | | | | |
| I.A | 16 | 1000:1 | 96 | 55 | 41 |
| Azoxystrobin | 0.016 | | | | |
| I.A | 1 | 4:1 | 99 | 24 | 75 |
| Benomyl | 0.25 | | | | |
| I.A | 0.25 | 1:1 | 54 | 21 | 33 |
| Benomyl | 0.25 | | | | |
| I.A | 4 | 16:1 | 96 | 28 | 68 |
| Benomyl | 0.25 | | | | |
| I.A | 4 | 250:1 | 94 | 73 | 21 |
| Benzovindiflupyr | 0.016 | | | | |
| I.A | 16 | 1000:1 | 100 | 81 | 19 |
| Benzovindiflupyr | 0.016 | | | | |
| I.A | 4 | 16:1 | 69 | 23 | 46 |
| Captan | 0.25 | | | | |
| I.A | 1 | 1:1 | 100 | 56 | 44 |
| Captan | 1 | | | | |
| I.A | 4 | 4:1 | 100 | 58 | 42 |
| Captan | 1 | | | | |
| I.A | 16 | 4:1 | 81 | 46 | 35 |
| Cyazofamid | 4 | | | | |
| I.A | 4 | 16:1 | 87 | 69 | 18 |
| Cyproconazol | 0.25 | | | | |
| I.A | 4 | 1:1 | 86 | 19 | 67 |
| Dimethomorph | 4 | | | | |
| I.A | 0.25 | 16:1 | 57 | 36 | 21 |
| Dimoxystrobin | 0.016 | | | | |
| I.A | 16 | 63:1 | 70 | 50 | 20 |
| Epoxiconazol | 0.25 | | | | |
| I.A | 4 | 16:1 | 51 | 30 | 21 |
| Epoxiconazol | 0.25 | | | | |
| I.A | 16 | 16:1 | 70 | 42 | 28 |
| Fenpropimorph | 1 | | | | |
| I.A | 4 | 4:1 | 100 | 18 | 82 |
| Fenpropimorph | 1 | | | | |
| I.A | 4 | 16:1 | 92 | 66 | 26 |
| Fludioxonil | 0.25 | | | | |
| I.A | 4 | 63:1 | 81 | 51 | 30 |
| Fludioxonil | 0.063 | | | | |
| I.A | 16 | 250:1 | 94 | 66 | 28 |
| Fludioxonil | 0.063 | | | | |
| I.A | 4 | 4:1 | 59 | 40 | 19 |
| Fluindapyr | 1 | | | | |
| I.A | 4 | 63:1 | 83 | 63 | 20 |
| Fluoxastrobin | 0.063 | | | | |
| I.A | 16 | 250:1 | 96 | 74 | 22 |
| Fluoxastrobin | 0.063 | | | | |
| I.A | 1 | 1:1 | 42 | 20 | 22 |
| Folpet | 1 | | | | |
| I.A | 4 | 4:1 | 51 | 24 | 27 |
| Folpet | 1 | | | | |
| I.A | 16 | 16:1 | 100 | 74 | 26 |
| Kresoximmethyl | 1 | | | | |
| I.A | 16 | 63:1 | 98 | 50 | 48 |
| Kresoximmethyl | 0.25 | | | | |
| I.A | 1 | 4:1 | 47 | 25 | 22 |
| Kresoximmethyl | 0.25 | | | | |
| I.A | 16 | 16:1 | 92 | 74 | 18 |
| Metominostrobin | 1 | | | | |
| I.A | 16 | 1:1 | 75 | 51 | 24 |
| Metrafenon | 16 | | | | |
| I.A | 4 | 16:1 | 49 | 24 | 25 |
| Penthiopyrad | 0.25 | | | | |
| I.A | 1 | 16:1 | 83 | 65 | 18 |
| Picoxystrobin | 0.063 | | | | |
| I.A | 16 | 250:1 | 99 | 77 | 22 |
| Picoxystrobin | 0.063 | | | | |
| I.A | 4 | 63:1 | 91 | 67 | 24 |
| Picoxystrobin | 0.063 | | | | |
| I.A | 16 | 63:1 | 62 | 44 | 18 |
| Prothioconazol | 0.25 | | | | |
| I.A | 4 | 4000:1 | 95 | 53 | 42 |
| Pyraclostrobin | 0.001 | | | | |
| I.A | 1 | 1000:1 | 93 | 50 | 43 |
| Pyraclostrobin | 0.001 | | | | |
| I.A | 0.25 | 250:1 | 89 | 48 | 41 |
| Pyraclostrobin | 0.001 | | | | |
| I.A | 16 | 63:1 | 72 | 50 | 22 |
| Tebuconazol | 0.25 | | | | |
| I.A | 4 | 250:1 | 65 | 36 | 29 |
| Trifloxystrobin | 0.016 | | | | |
| I.A | 4 | 16:1 | 48 | 29 | 19 |
| Tebuconazol | 0.25 | | | | |
| I.A | 16 | 1000: 1 | 98 | 55 | 43 |
| Trifloxystrobin | 0.016 | | | | |

V) Fungicidal Activity Against Septoria tritici

The active compounds were formulated separately as a stock solution having a concentration of 10000 ppm in dimethyl sulfoxide. The stock solutions were mixed according to the ratio, pipetted onto a micro titer plate (MTP) and diluted with water to the stated concentrations. A spore suspension of Septoria tritici in an aqueous biomalt or yeast-bactopeptone-sodiumacetate solution was then added. The plates were placed in a water vapor-saturated chamber at a temperature of 18° C. Using an absorption photometer, the MTPs were measured at 405 nm 7 days after the inoculation. The measured parameters were compared to the growth of the active compound-free control variant (100%) and the fungus-free and active compound-free blank value to determine the relative growth in % of the pathogens in the respective active compounds. These percentages were converted into efficacies.

TABLE V.1
Use examples of fungicidal activity against Septoria tritici
of I.H/I.J as component A and components B

| | | Concentration | |
|---|---|---|---|
| Active mixture | Code of | A + B | Efficacy |
| (component A + B) | component B | (ppm) | (ppm) |
| I.H/I.J + component B | A.1.34 | 16 + 63 | 79 |
| I.H/I.J + Boscalid | A.3.4 | 16 + 4 | 100 |
| I.H/I.J + Chlorothalonil | H.3.2 | 16 + 1 | 100 |
| I.H/I.J + Cyproconazol | B.1.4 | 16 + 16 | 100 |
| I.H/I.J + Difenoconazol | B.1.5 | 16 + 0.25 | 96 |
| I.H/I.J + Dimoxystrobin | A.1.4 | 16 + 1 | 97 |
| I.H/I.J + Epoxiconazol | B.1.8. | 16 + 0.25 | 96 |
| I.H/I.J + Fenpropidin | B.2.6 | 16 + 4 | 96 |
| I.H/I.J + Florylpicoxamid | A.2.6 | 16 + 1 | 90 |
| I.H/I.J + Kresoxim-methyl | A.1.9 | 16 + 1 | 90 |
| I.H/I.J + Pydiflumetofen | A.3.17 | 16 + 16 | 91 |
| I.H/I.J + Sulfur | H.1.7 | 16 + 63 | 96 |
| I.H/I.J + Triticonazol | B.1.29 | 16 + 1 | 92 |

TABLE V.2
Use examples of fungicidal activity against Septoria tritici
of I.F as component A and components B

| | | Concentration | |
|---|---|---|---|
| Active mixture | Code of | A + B | Efficacy |
| (component A + B) | component B | (ppm) | (ppm) |
| I.F + component B | A.1.34 | 4 + 63 | 100 |
| I.F + component B | A.1.25 | 4 + 0.016 | 75 |
| I.F + component B | A.3.29 | 4 + 1 | 100 |
| I.F + component B | A.3.34 | 4 + 1 | 96 |
| I.F + component B | A.3.32 | 4 + 1 | 98 |
| I.F + Azoxystrobin | A.1.1 | 4 + 0.063 | 53 |
| I.F + Benzovindiflupyr | A.3.2 | 4 + 0.063 | 54 |
| I.F + Bixafen | A.3.3 | 4 + 0.25 | 72 |
| I.F + Boscalid | A.3.4 | 4 + 4 | 96 |
| I.F + Captan | H.3.4 | 4 + 4 | 100 |
| I.F + Chlorothalonil | H.3.2 | 4 + 1 | 99 |
| I.F + Cyazofamid | A.2.1 | 4 + 16 | 61 |
| I.F + Cyproconazol | B.1.4 | 4 + 16 | 93 |
| I.F + Difenoconazol | B.1.5 | 4 + 0.25 | 92 |
| I.F + Dimoxystrobin | A. 1.4 | 4 + 1 | 100 |
| I.F + Dithianon | H.4.9 | 4 + 1 | 100 |
| I.F + Epoxiconazol | B.1.8. | 4 + 0.25 | 91 |
| I.F + Fluindapyr | A.3.28 | 4 + 1 | 91 |
| I.F + Fluquinconazol | B.1.10 | 4 + 1 | 94 |
| I.F + component B | A.3.24 | 4 + 1 | 98 |
| I.F + Mefentrifluconazol | B.1.38 | 4 + 0.016 | 94 |
| I.F + Metconazol | B.1.17 | 4 + 0.063 | 77 |
| I.F + Penthiopyrad | A.3.16 | 4 + 0.25 | 88 |
| I.F + Picoxystrobin | A.1.13 | 4 + 0.25 | 100 |
| I.F + Propiconazol | B.1.22 | 4 + 0.25 | 85 |
| I.F + Prothioconazol | B.1.23 | 4 + 0.25 | 85 |
| I.F + Pyraclostrobin | A.1.14 | 4 + 0.016 | 100 |
| I.F + Trifloxystrobin | A.1.17 | 4 + 0.063 | 100 |
| I.F + Triticonazol | B.1.29 | 4 + 1 | 95 |

TABLE V.3
Use examples of fungicidal activity against Septoria tritici
of I.B/I.C as component A and components B

| | | Concentration | |
|---|---|---|---|
| Active mixture | Code of | A + B | Efficacy |
| (component A + B) | component B | (ppm) | (ppm) |
| I.B/I.C + component B | A.1.34 | 1 + 63 | 100 |
| I.B/I.C + component B | A.1.25 | 1 + 0.016 | 53 |
| I.B/I.C + component B | A.3.29 | 1 + 1 | 100 |
| I.B/I.C + component B | A.2.3 | 1 + 1 | 100 |
| I.B/I.C + component B | A.2.5 | 1 + 1 | 100 |
| I.B/I.C + component B | K.1.29 | 1 + 16 | 99 |
| I.B/I.C + component B | A.3.34 | 1 + 1 | 100 |
| I.B/I.C + component B | A.3.32 | 1 + 1 | 99 |
| I.B/I.C + Azoxystrobin | A.1.1 | 1 + 0.063 | 99 |
| I.B/I.C + Benzovindiflupyr | A.3.2 | 1 + 0.063 | 96 |
| I.B/I.C + Bixafen | A.3.3 | 1 + 0.25 | 98 |
| I.B/I.C + Boscalid | A.3.4 | 1 + 4 | 99 |
| I.B/I.C + Captan | H.3.4 | 1 + 4 | 100 |
| I.B/I.C + Chlorothalonil | H.3.2 | 1 + 1 | 99 |
| I.B/I.C + Cyazofamid | A.2.1 | 1 + 16 | 50 |
| I.B/I.C + Cyflufenamid | K.1.3 | 1 + 63 | 52 |
| I.B/I.C + Cyproconazol | B.1.4 | 1 + 16 | 99 |
| I.B/I.C + Difenoconazol | B.1.5 | 1 + 0.25 | 100 |
| I.B/I.C + Dimoxystrobin | A.1.4 | 1 + 1 | 100 |
| I.B/I.C + Dithianon | H.4.9 | 1 + 1 | 99 |
| I.B/I.C + Epoxiconazol | B.1.8. | 1 + 0.25 | 100 |
| I.B/I.C + Fenpropidin | B.2.6 | 1 + 4 | 100 |
| I.B/I.C + Fenpropimorph | B.2.4 | 1 + 1 | 82 |
| I.B/I.C + Florylpicoxamid | A.2.6 | 1 + 1 | 100 |
| I.B/I.C + Fluindapyr | A.3.28 | 1 + 1 | 94 |
| I.B/I.C + Fluopyram | A.3.7 | 1 + 0.25 | 79 |
| I.B/I.C + Fluquinconazol | B.1.10 | 1 + 1 | 100 |
| I.B/I.C + Fluxapyroxad | A.3.9 | 1 + 0.063 | 83 |
| I.B/I.C + component B | A.3.24 | 1 + 1 | 95 |
| I.B/I.C + Kresoxim-methyl | A.1.9 | 1 + 1 | 99 |
| I.B/I.C + Mefentrifluconazol | B.1.38 | 1 + 0.016 | 100 |
| I.B/I.C + Metconazol | B.1.17 | 1 + 0.063 | 98 |
| I.B/I.C + Penthiopyrad | A.3.16 | 1 + 0.25 | 97 |
| I.B/I.C + Picoxystrobin | A.1.13 | 1 + 0.25 | 98 |
| I.B/I.C + Propiconazol | B.1.22 | 1 + 0.25 | 100 |
| I.B/I.C + Prothioconazol | B.1.23 | 1 + 0.25 | 99 |
| I.B/I.C + Pydiflumetofen | A.3.17 | 1 + 16 | 100 |
| I.B/I.C + Pyraclostrobin | A.1.14 | 1 + 0.016 | 100 |
| I.B/I.C + Sulfur | H.1.7 | 1 + 63 | 100 |
| I.B/I.C + Tebuconazol | B.1.25 | 1 + 0.25 | 96 |
| I.B/I.C + Trifloxystrobin | A.1.17 | 1 + 0.063 | 100 |
| I.B/I.C + Triticonazol | B.1.29 | 1 + 1 | 97 |

TABLE V.4
Use examples fungicidal activity against
Septoria tritici of selected components B

| Code of | Common name | Concentration | Observed |
|---|---|---|---|
| component B | component B | (ppm) | efficacy |
| A.3.29 | | 0.063 | 68 |
| A.2.3 | | 0.016 | 52 |
| A.2.5 | | 0.063 | 33 |
| K.1.29 | | 4 | 6 |
| A.3.34 | | 0.25 | 17 |
| A.3.32 | | 1 | 70 |
| | | 0.25 | 22 |
| K.1.30 | | 4 | 0 |
| | | 1 | 0 |
| K.1.32 | | 1 | 0 |
| | | 0.25 | 0 |
| K.1.31 | | 4 | 62 |
| | | 1 | 5 |
| K.1.54 | | 1 | 3 |
| A.1.25 | | 0.016 | 25 |
| | | 0.004 | 3 |
| A.1.1 | Azoxystrobin | 0.063 | 28 |
| | | 0.016 | 4 |
| A.3.2 | Benzovindiflupyr | 0.063 | 35 |
| | | 0.016 | 17 |
| A.3.3 | Bixafen | 0.25 | 41 |
| | | 0.063 | 16 |
| A.3.4 | Boscalid | 1 | 61 |
| H.3.4 | Captan | 0.25 | 22 |
| H.3.2 | Chlorothalonil | 0.25 | 25 |
| A.2.1 | Cyazofamid | 16 | 8 |
| | | 4 | 2 |
| B.1.5 | Difenoconazol | 0.016 | 4 |
| G.3.1 | Dimethomorph | 4 | 11 |
| A.1.4 | Dimoxystrobin | 0.063 | 35 |
| B.1.8. | Epoxiconazol | 0.063 | 52 |
| B.2.6 | Fenpropidin | 1 | 1 |
| B.2.4 | Fenpropimorph | 0.25 | 0 |
| A.2.6 | Florylpicoxamid | 0.25 | 68 |
| F.1.5 | Fludioxonil | 0.063 | 5 |
| A.3.28 | Fluindapyr | 1 | 58 |
| | | 0.25 | 21 |
| A.3.7 | Fluopyram | 0.25 | 33 |
| | | 0.063 | 13 |
| A.1.8 | Fluoxastrobin | 0.25 | 32 |
| | | 0.063 | 3 |
| A.3.9 | Fluxapyroxad | 0.063 | 10 |
| | | 0.016 | 12 |
| H.3.5 | Folpet | 1 | 70 |
| A.3.24 | | 1 | 76 |
| | | 0.25 | 25 |
| A.3.29 | Isoflucypram | 1 | 62 |
| | | 0.25 | 61 |
| A.1.9 | Kresoximmethyl | 0.063 | 41 |
| H.2.2 | Mancozeb | 1 | 9 |
| | | 0.25 | 7 |
| B.1.38 | Mefentrifluconazol | 0.00025 | 6 |
| A.1.11 | Metominostrobin | 1 | 46 |
| D.2.6 | Metrafenon | 16 | 1 |
| | | 4 | 1 |
| A.3.16 | Penthiopyrad | 0.25 | 28 |
| | | 0.063 | 5 |
| A.1.13 | Picoxystrobin | 0.063 | 34 |
| B.1.22 | Propiconazol | 0.063 | 17 |
| A.1.14 | Pyraclostrobin | 0.000063 | 21 |
| A.1.17 | Trifloxystrobin | 0.016 | 43 |

TABLE V.5
Use examples of synergistic fungicidal activity against Septoria tritici
of compound I.M as component A and components B

| | | | | Calculated | |
|---|---|---|---|---|---|
| Active | | | | efficacy | |
| compound/ | Concen- | | | according | Syner- |
| active mixture | tration | Ratio | Observed | to Colby | gism |
| A + B | (ppm) | A:B | efficacy | (%) | (%) |
| I.M | 4 | — | 42 | | |
| | 1 | — | 35 | | |
| | 0.25 | — | 23 | | |
| | 0.063 | — | 14 | | |
| I.M | 1 | 16:1 | 94 | 76 | 18 |
| A.3.29 | 0.063 | | | | |
| I.M | 4 | 1:1 | 70 | 46 | 24 |
| K.1.29 | 4 | | | | |
| I.M | 1 | 1:4 | 57 | 30 | 27 |
| K.1.29 | 4 | | | | |
| I.M | 4 | 16:1 | 96 | 52 | 44 |
| A.2.5 | 0.25 | | | | |
| I.M | 1 | 4:1 | 73 | 38 | 35 |
| A.2.5 | 0.25 | | | | |
| I.M | 1 | 1:1 | 96 | 78 | 18 |
| A.3.32 | 1 | | | | |
| I.M | 4 | 16:1 | 90 | 55 | 35 |
| A.3.32 | 0.25 | | | | |
| I.M | 1 | 1:1 | 52 | 29 | 23 |
| K.1.31 | 1 | | | | |
| I.M | 4 | 250:1 | 100 | 57 | 43 |
| A.1.25 | 0.016 | | | | |
| I.M | 4 | 1000:1 | 90 | 44 | 46 |
| A.1.25 | 0.004 | | | | |
| I.M | 4 | 63:1 | 100 | 58 | 42 |
| Azoxystrobin | 0.063 | | | | |
| I.M | 1 | 16:1 | 82 | 46 | 36 |
| Azoxystrobin | 0.063 | | | | |
| I.M | 4 | 250:1 | 90 | 45 | 45 |
| Azoxystrobin | 0.016 | | | | |
| I.M | 4 | 63:1 | 98 | 63 | 35 |
| Benzovindiflupyr | 0.063 | | | | |
| I.M | 1 | 16:1 | 85 | 52 | 33 |
| Benzovindiflupyr | 0.063 | | | | |
| I.M | 4 | 250:1 | 82 | 52 | 30 |
| Benzovindiflupyr | 0.016 | | | | |
| I.M | 4 | 16:1 | 99 | 66 | 33 |
| Bixafen | 0.25 | | | | |
| I.M | 1 | 4:1 | 93 | 56 | 37 |
| Bixafen | 0.25 | | | | |
| I.M | 4 | 63:1 | 91 | 52 | 39 |
| Bixafen | 0.063 | | | | |
| I.M | 1 | 1:1 | 100 | 71 | 29 |
| Boscalid | 1 | | | | |
| I.M | 4 | 4:1 | 100 | 77 | 23 |
| Boscalid | 1 | | | | |
| I.M | 0.25 | 1:4 | 97 | 70 | 27 |
| Boscalid | 1 | | | | |
| I.M | 1 | 4:1 | 100 | 44 | 56 |
| Chlorothalonil | 0.25 | | | | |
| I.M | 4 | 16:1 | 100 | 56 | 44 |
| Chlorothalonil | 0.25 | | | | |
| I.M | 4 | 1:4 | 98 | 47 | 51 |
| Cyazofamid | 16 | | | | |
| I.M | 1 | 1:16 | 59 | 31 | 28 |
| Cyazofamid | 16 | | | | |
| I.M | 1 | 16:1 | 90 | 52 | 38 |
| Dimoxystrobin | 0.063 | | | | |
| I.M | 4 | 63:1 | 96 | 72 | 24 |
| Epoxiconazol | 0.063 | | | | |
| I.M | 4 | 4:1 | 61 | 43 | 18 |
| Fenpropidin | 1 | | | | |
| I.M | 4 | 16:1 | 62 | 42 | 20 |
| Fenpropimorph | 0.25 | | | | |
| I.M | 1 | 4:1 | 94 | 76 | 18 |
| Florylpicoxamid | 0.25 | | | | |
| I.M | 4 | 16:1 | 100 | 82 | 18 |
| Florylpicoxamid | 0.25 | | | | |
| I.M | 1 | 16:1 | 50 | 30 | 20 |
| Fludioxonil | 0.063 | | | | |
| I.M | 4 | 4:1 | 99 | 76 | 23 |
| Fluindapyr | 1 | | | | |
| I.M | 4 | 16:1 | 85 | 54 | 31 |
| Fluindapyr | 0.25 | | | | |
| I.M | 1 | 1:1 | 92 | 68 | 24 |
| Fluindapyr | 1 | | | | |
| I.M | 4 | 63:1 | 70 | 50 | 20 |
| Fluopyram | 0.063 | | | | |
| I.M | 4 | 16:1 | 96 | 61 | 35 |
| Fluopyram | 0.25 | | | | |
| I.M | 1 | 4:1 | 68 | 50 | 18 |
| Fluopyram | 0.25 | | | | |
| I.M | 4 | 63:1 | 83 | 44 | 39 |
| Fluoxastrobin | 0.063 | | | | |
| I.M | 4 | 16:1 | 100 | 61 | 39 |
| Fluoxastrobin | 0.25 | | | | |
| I.M | 1 | 4:1 | 68 | 50 | 18 |
| Fluoxastrobin | 0.25 | | | | |
| I.M | 4 | 63:1 | 87 | 48 | 39 |
| Fluxapyroxad | 0.063 | | | | |
| I.M | 4 | 250:1 | 67 | 49 | 18 |
| Fluxapyroxad | 0.016 | | | | |
| I.M | 1 | 1:1 | 99 | 78 | 21 |
| Folpet | 1 | | | | |
| I.M | 0.25 | 1:4 | 97 | 77 | 20 |
| Folpet | 1 | | | | |
| I.M | 4 | 16:1 | 95 | 57 | 38 |
| A.3.24 | 0.25 | | | | |
| I.M | 4 | 16:1 | 100 | 77 | 23 |
| Isoflucypram | 0.25 | | | | |
| I.M | 1 | 1:1 | 100 | 71 | 29 |
| Isoflucypram | 1 | | | | |
| I.M | 4 | 16:1 | 72 | 47 | 25 |
| Mancozeb | 0.25 | | | | |
| I.M | 4 | 4:1 | 82 | 47 | 35 |
| Mancozeb | 1 | | | | |
| I.M | 0.063 | 250:1 | 51 | 19 | 32 |
| Mefentrifluconazol | 0.00025 | | | | |
| I.M | 4 | 4:1 | 99 | 69 | 30 |
| Metominostrobin | 1 | | | | |
| I.M | 1 | 1:16 | 50 | 26 | 24 |
| Metrafenon | 16 | | | | |
| I.M | 4 | 16:1 | 98 | 59 | 39 |
| Penthiopyrad | 0.25 | | | | |
| I.M | 1 | 4:1 | 98 | 47 | 51 |
| Penthiopyrad | 0.25 | | | | |
| I.M | 4 | 63:1 | 92 | 45 | 47 |
| Penthiopyrad | 0.063 | | | | |
| I.M | 4 | 63:1 | 100 | 62 | 38 |
| Picoxystrobin | 0.063 | | | | |
| I.M | 1 | 16:1 | 98 | 51 | 47 |
| Picoxystrobin | 0.063 | | | | |
| I.M | 0.25 | 4:1 | 91 | 49 | 42 |
| Picoxystrobin | 0.063 | | | | |
| I.M | 1 | 16:1 | 57 | 38 | 19 |
| Propiconazol | 0.063 | | | | |
| I.M | 1 | 63:1 | 89 | 58 | 31 |
| Trifloxystrobin | 0.016 | | | | |
| I.M | 4 | 250:1 | 100 | 67 | 33 |
| Trifloxystrobin | 0.016 | | | | |

TABLE V.6
Use examples of synergistic fungicidal activity against Septoria tritici
of compound I.E as component A and components B

| | | | | Calculated | |
|---|---|---|---|---|---|
| Active | | | | efficacy | |
| compound/ | Concen- | | | according | Syner- |
| active mixture | tration | Ratio | Observed | to Colby | gism |
| A + B | (ppm) | A:B | efficacy | (%) | (%) |
| I.E | 63 | — | 14 | | |
| | 16 | — | 13 | | |
| | 4 | — | 7 | | |
| | 0.25 | — | 13 | | |
| I.E | 4 | 63:1 | 62 | 38 | 24 |
| A.2.5 | 0.063 | | | | |
| I.E | 16 | 250:1 | 59 | 37 | 22 |
| Azoxystrobin | 0.063 | | | | |
| I.E | 16 | 63:1 | 54 | 32 | 22 |
| Captan | 0.25 | | | | |
| I.E | 63 | 16:1 | 78 | 15 | 63 |
| Cyazofamid | 4 | | | | |
| I.E | 16 | 16:1 | 84 | 63 | 21 |
| Fluindapyr | 1 | | | | |
| I.E | 63 | 63:1 | 92 | 67 | 25 |
| Isoflucypram | 1 | | | | |
| I.E | 16 | 16:1 | 97 | 66 | 31 |
| Isoflucypram | 1 | | | | |
| I.E | 63 | 250:1 | 98 | 66 | 32 |
| Isoflucypram | 0.25 | | | | |
| I.E | 0.25 | 4000:1 | 57 | 32 | 25 |
| Pyraclostrobin | 0.000063 | | | | |

TABLE V.7
Use examples of synergistic fungicidal activity against Septoria tritici
of compound I.E as component A and components B

| | | | | Calculated | |
|---|---|---|---|---|---|
| Active | | | | efficacy | |
| compound/ | Concen- | | | according | Syner- |
| active mixture | tration | Ratio | Observed | to Colby | gism |
| A + B | (ppm) | A:B | efficacy | (%) | (%) |
| I.A | 16 | — | 19 | | |
| | 4 | — | 8 | | |
| | 1 | — | 0 | | |
| | 0.25 | — | 2 | | |
| | 0.063 | — | 3 | | |
| I.A | 1 | 16:1 | 95 | 68 | 27 |
| A.3.29 | 0.063 | | | | |
| I.A | 0.25 | 16:1 | 91 | 53 | 38 |
| A.2.3 | 0.016 | | | | |
| I.A | 1 | 16:1 | 100 | 34 | 66 |
| A.2.5 | 0.063 | | | | |
| I.A | 4 | 1:1 | 79 | 13 | 66 |
| K.1.29 | 4 | | | | |
| I.A | 1 | 1:4 | 53 | 6 | 47 |
| K.1.29 | 4 | | | | |
| I.A | 16 | 4:1 | 92 | 24 | 68 |
| K.1.29 | 4 | | | | |
| I.A | 4 | 16:1 | 82 | 23 | 59 |
| A.2.5 | 0.25 | | | | |
| I.A | 1 | 4:1 | 55 | 17 | 38 |
| A.2.5 | 0.25 | | | | |
| I.A | 16 | 63:1 | 85 | 33 | 52 |
| A.2.5 | 0.25 | | | | |
| I.A | 16 | 16:1 | 100 | 76 | 24 |
| A.3.32 | 1 | | | | |
| I.A | 4 | 4:1 | 94 | 73 | 21 |
| A.3.32 | 1 | | | | |
| I.A | 16 | 63:1 | 79 | 37 | 42 |
| A.3.32 | 0.25 | | | | |
| I.A | 4 | 1:1 | 27 | 8 | 19 |
| K.1.30 | 4 | | | | |
| I.A | 16 | 16:1 | 37 | 19 | 18 |
| K.1.30 | 1 | | | | |
| I.A | 4 | 4:1 | 29 | 8 | 21 |
| K.1.32 | 1 | | | | |
| I.A | 16 | 63:1 | 41 | 19 | 22 |
| K.1.32 | 0.25 | | | | |
| I.A | 16 | 4:1 | 100 | 70 | 30 |
| K.1.31 | 4 | | | | |
| I.A | 4 | 1:1 | 90 | 65 | 25 |
| K.1.31 | 4 | | | | |
| I.A | 16 | 16:1 | 45 | 24 | 21 |
| K.1.31 | 1 | | | | |
| I.A | 4 | 4:1 | 99 | 10 | 89 |
| K.1.54 | 1 | | | | |
| I.A | 16 | 1000:1 | 99 | 39 | 60 |
| A.1.25 | 0.016 | | | | |
| I.A | 4 | 250:1 | 57 | 31 | 26 |
| A.1.25 | 0.016 | | | | |
| I.A | 16 | 4000:1 | 41 | 22 | 19 |
| A.1.25 | 0.004 | | | | |
| I.A | 16 | 250:1 | 99 | 42 | 57 |
| Azoxystrobin | 0.063 | | | | |
| I.A | 4 | 63:1 | 70 | 33 | 37 |
| Azoxystrobin | 0.063 | | | | |
| I.A | 16 | 1000:1 | 75 | 23 | 52 |
| Azoxystrobin | 0.016 | | | | |
| I.A | 16 | 250:1 | 94 | 48 | 46 |
| Benzovindiflupyr | 0.063 | | | | |
| I.A | 16 | 1000:1 | 61 | 33 | 28 |
| Benzovindiflupyr | 0.016 | | | | |
| I.A | 4 | 63:1 | 73 | 40 | 33 |
| Benzovindiflupyr | 0.063 | | | | |
| I.A | 16 | 63:1 | 100 | 53 | 47 |
| Bixafen | 0.25 | | | | |
| I.A | 16 | 250:1 | 85 | 33 | 52 |
| Bixafen | 0.063 | | | | |
| I.A | 4 | 16:1 | 90 | 46 | 44 |
| Bixafen | 0.25 | | | | |
| I.A | 4 | 4:1 | 100 | 64 | 36 |
| Boscalid | 1 | | | | |
| I.A | 1 | 1:1 | 97 | 61 | 36 |
| Boscalid | 1 | | | | |
| I.A | 16 | 16:1 | 97 | 69 | 28 |
| Boscalid | 1 | | | | |
| I.A | 4 | 16:1 | 100 | 30 | 70 |
| Chlorothalonil | 0.25 | | | | |
| I.A | 1 | 4:1 | 76 | 25 | 51 |
| Chlorothalonil | 0.25 | | | | |
| I.A | 16 | 63:1 | 98 | 39 | 59 |
| Chlorothalonil | 0.25 | | | | |
| I.A | 16 | 1:1 | 51 | 26 | 25 |
| Cyazofamid | 16 | | | | |
| I.A | 4 | 250:1 | 57 | 12 | 45 |
| Difenoconazol | 0.016 | | | | |
| I.A | 1 | 16:1 | 46 | 4 | 42 |
| Difenoconazol | 0.063 | | | | |
| I.A | 4 | 1:1 | 56 | 18 | 38 |
| Dimethomorph | 4 | | | | |
| I.A | 1 | 16:1 | 97 | 35 | 62 |
| Dimoxystrobin | 0.063 | | | | |
| I.A | 4 | 63:1 | 92 | 40 | 52 |
| Dimoxystrobin | 0.063 | | | | |
| I.A | 16 | 250:1 | 93 | 62 | 31 |
| Epoxiconazol | 0.063 | | | | |
| I.A | 4 | 63:1 | 82 | 56 | 26 |
| Epoxiconazol | 0.063 | | | | |
| I.A | 4 | 16:1 | 99 | 71 | 28 |
| Florylpicoxamid | 0.25 | | | | |
| I.A | 16 | 63:1 | 100 | 75 | 25 |
| Florylpicoxamid | 0.25 | | | | |
| I.A | 4 | 63:1 | 43 | 13 | 30 |
| Fludioxonil | 0.063 | | | | |
| I.A | 1 | 16:1 | 29 | 6 | 23 |
| Fludioxonil | 0.063 | | | | |
| I.A | 16 | 16:1 | 100 | 66 | 34 |
| Fluindapyr | 1 | | | | |
| I.A | 4 | 4:1 | 87 | 61 | 26 |
| Fluindapyr | 1 | | | | |
| I.A | 16 | 63:1 | 55 | 36 | 19 |
| Fluindapyr | 0.25 | | | | |
| I.A | 16 | 63:1 | 84 | 46 | 38 |
| Fluopyram | 0.25 | | | | |
| I.A | 4 | 16:1 | 65 | 38 | 27 |
| Fluopyram | 0.25 | | | | |
| I.A | 16 | 63:1 | 100 | 46 | 54 |
| Fluoxastrobin | 0.25 | | | | |
| I.A | 4 | 16:1 | 77 | 38 | 39 |
| Fluoxastrobin | 0.25 | | | | |
| I.A | 16 | 250:1 | 76 | 22 | 54 |
| Fluoxastrobin | 0.063 | | | | |
| I.A | 4 | 63:1 | 53 | 17 | 36 |
| Fluxapyroxad | 0.063 | | | | |
| I.A | 16 | 250:1 | 63 | 28 | 35 |
| Fluxapyroxad | 0.063 | | | | |
| I.A | 1 | 1:1 | 100 | 70 | 30 |
| Folpet | 1 | | | | |
| I.A | 4 | 4:1 | 100 | 72 | 28 |
| Folpet | 1 | | | | |
| I.A | 16 | 63:1 | 87 | 40 | 47 |
| A.3.24 | 0.25 | | | | |
| I.A | 16 | 16:1 | 100 | 81 | 19 |
| A.3.24 | 1 | | | | |
| I.A | 1 | 4:1 | 55 | 25 | 30 |
| A.3.24 | 0.25 | | | | |
| I.A | 4 | 16:1 | 100 | 64 | 36 |
| Isoflucypram | 0.25 | | | | |
| I.A | 16 | 63:1 | 100 | 68 | 32 |
| Isoflucypram | 0.25 | | | | |
| I.A | 1 | 16:1 | 91 | 41 | 50 |
| Kresoximmethyl | 0.063 | | | | |
| I.A | 4 | 63:1 | 99 | 46 | 53 |
| Kresoximmethyl | 0.063 | | | | |
| I.A | 16 | 63:1 | 44 | 25 | 19 |
| Mancozeb | 0.25 | | | | |
| I.A | 0.25 | 1000:1 | 48 | 8 | 40 |
| Mefentrifluconazol | 0.00025 | | | | |
| I.A | 1 | 4000:1 | 43 | 6 | 37 |
| Mefentrifluconazol | 0.00025 | | | | |
| I.A | 4 | 4:1 | 91 | 51 | 40 |
| Metominostrobin | 1 | | | | |
| I.A | 16 | 16:1 | 100 | 57 | 43 |
| Metominostrobin | 1 | | | | |
| I.A | 16 | 1:1 | 72 | 20 | 52 |
| Metrafenon | 16 | | | | |
| I.A | 4 | 1:4 | 78 | 8 | 70 |
| Metrafenon | 16 | | | | |
| I.A | 16 | 4:1 | 60 | 20 | 40 |
| Metrafenon | 4 | | | | |
| I.A | 4 | 16:1 | 93 | 34 | 59 |
| Penthiopyrad | 0.25 | | | | |
| I.A | 16 | 250:1 | 78 | 24 | 54 |
| Penthiopyrad | 0.063 | | | | |
| I.A | 16 | 63:1 | 97 | 42 | 55 |
| Penthiopyrad | 0.25 | | | | |
| I.A | 4 | 63:1 | 94 | 39 | 55 |
| Picoxystrobin | 0.063 | | | | |
| I.A | 16 | 250:1 | 100 | 47 | 53 |
| Picoxystrobin | 0.063 | | | | |
| I.A | 16 | 250:1 | 54 | 33 | 21 |
| Propiconazol | 0.063 | | | | |
| I.A | 1 | 16:1 | 40 | 17 | 23 |
| Propiconazol | 0.063 | | | | |
| I.A | 0.063 | 1000:1 | 91 | 23 | 68 |
| Pyraclostrobin | 0.000063 | | | | |
| I.A | 4 | 250:1 | 100 | 48 | 52 |
| Trifloxystrobin | 0.016 | | | | |
| I.A | 16 | 1000:1 | 99 | 54 | 45 |
| Trifloxystrobin | 0.016 | | | | |

1-10. (canceled)

11: A fungicidal mixture comprising as active components
1) at least one active compound of the formula I, or an N-oxide, or an agriculturally useful salt thereof, 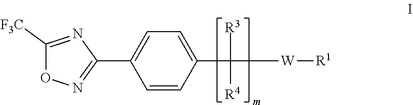
wherein:
W is —(C═O)—NR²-#, —(C═S)—NR²-#, —NR²—(C═O)-# or —NR²—(C═S)-#; wherein # denotes the position, which is attached to the group R¹;
R¹ is C₁-C₆-alkyl, C₃-C₈-cycloalkyl, C₃-C₈-cycloalkenyl, C₂-C₆-alkenyl, C₂-C₆-alkynyl, C₁-C₆-alkoxyimino-C₁-C₄-alkyl or phenyl; wherein the phenyl ring is unsubstituted or substituted with 1, 2, 3 or up to the maximum possible number of identical or different radicals selected from the group consisting of halogen, C₁-C₆-alkyl and C₁-C₆-alkoxy;
R² is hydrogen, C₁-C₆-alkyl, C₂-C₆-alkenyl, C₂-C₆-alkynyl, C₁-C₆-alkoxy, C₃-C₈-cycloalkyl, C₃-C₈-cycloalkenyl, and C₃-C₈-cycloalkyl-C₁-C₄-alkyl; and wherein any of the aliphatic or cyclic groups are unsubstituted or substituted with 1, 2, 3 or up to the maximum possible number of identical or different radicals selected from the group consisting of halogen, hydroxy, oxo, cyano, C₁-C₆-alkyl, C₁-C₆-alkoxy and C₃-C₈-cycloalkyl; or
R¹ and R² together with the nitrogen atom to which they are bound form a saturated or partially unsaturated mono- or bicyclic 3- to 7-membered heterocycle, wherein the heterocycle includes beside one nitrogen atom and one or more carbon atoms no further heteroatom or 1, 2 or 3 further heteroatoms independently selected from N, O and S as ring member atoms; and wherein one or two CH₂ groups of the heterocycle may be replaced by one or two groups independently selected from the group of C(═O) and C(═S); and wherein the heterocycle is unsubstituted or carries 1, 2, 3, 4 or up to the maximum possible number of identical or different radicals selected from the group consisting of halogen, cyano, C₁-C₆-alkyl, C₁-C₆-haloalkyl, C₁-C₆-alkoxy,
m is 0 or 1;
R³, R⁴ independently of each other are selected from the group consisting of hydrogen, halogen, cyano, C₁-C₄-alkyl, C₁-C₄-alkenyl, C₁-C₄-alkynyl, C₁-C₄-haloalkyl and C₁-C₄-alkoxy;
or R³ and R⁴ together with the carbon atom to which they are bound form a cyclopropyl ring;
and as component
2) at least one active compound II selected from groups A) to O), or an N-oxide, or an agriculturally useful salt thereof:
A) Respiration inhibitors:
Inhibitors of complex III at Qₒ site: azoxystrobin (A.1.1), coumethoxystrobin (A.1.2), coumoxystrobin (A.1.3), dimoxystrobin (A.1.4), enestroburin (A.1.5), fenaminstrobin (A.1.6), fenoxystrobin/flufenoxystrobin (A.1.7), fluoxastrobin (A.1.8), kresoxim-methyl (A.1.9), mandestrobin (A.1.10), metominostrobin (A.1.11), orysastrobin (A.1.12), picoxystrobin (A.1.13), pyraclostrobin (A.1.14), pyrametostrobin (A.1.15), pyraoxystrobin (A.1.16), trifloxystrobin (A.1.17), 2-(2-(3-(2,6-dichlorophenyl)-1-methyl-allylideneaminooxymethyl)-phenyl)-2-methoxyimino-N-methyl-acetamide (A.1.18), pyribencarb (A.1.19), triclopyricarb/chlorodincarb (A.1.20), famoxadone (A.1.21), fenamidone (A.1.21a), methyl-N-[2-[(1,4-dimethyl-5-phenyl-pyrazol-3-yl)oxylmethyl]phenyl]-N-methoxy-carbamate (A.1.22), 1-[3-chloro-2-[[1-(4-chlorophenyl)-1H-pyrazol-3-yl]oxymethyl]phenyl]-4-methyl-tetrazol-5-one (A.1.23), 1-[3-bromo-2-[[1-(4-chlorophenyl)pyrazol-3-yl]oxymethyl]phenyl]-4-methyl-tetrazol-5-one (A.1.24), 1-[2-[[1-(4-chlorophenyl)pyrazol-3-yl]oxymethyl]-3-methyl-phenyl]-4-methyl-tetrazol-5-one (A.1.25), 1-[2-[[1-(4-chlorophenyl)pyrazol-3-yl]oxymethyl]-3-fluoro-phenyl]-4-methyl-tetrazol-5-one (A.1.26), 1-[2-[[1-(2,4-dichloro-phenyl)pyrazol-3-yl]oxymethyl]-3-fluoro-phenyl]-4-methyl-tetrazol-5-one (A.1.27), 1-[2-[[4-(4-chlorophenyl)thiazol-2-yl]oxymethyl]-3-methyl-phenyl]-4-methyl-tetrazol-5-one (A.1.28), 1-[3-chloro-2-[[4-(p-tolyl)thiazol-2-yl]oxymethyl]phenyl]-4-methyl-tetrazol-5-one (A.1.29), 1-[3-cyclopropyl-2-[[2-methyl-4-(1-methylpyrazol-3-yl)phenoxy]methyl]phenyl]-4-methyl-tetrazol-5-one (A.1.30), 1-[3-(difluoromethoxy)-2-[[2-methyl-4-(1-methylpyrazol-3-yl)phenoxy]methyl]phenyl]-4-methyl-tetrazol-5-one (A.1.31), 1-methyl-4-[3-methyl-2-[[2-methyl-4-(1-methylpyrazol-3-yl)phenoxy]methyl]phenyl]tetrazol-5-one (A.1.32), 1-methyl-4-[3-methyl-2-[[1-[3-(trifluoromethyl)phenyl]-ethylideneamino]oxymethyl]phenyl]tetrazol-5-one (A.1.33), (Z,2E)-5-[1-(2,4-dichlorophenyl)pyrazol-3-yl]-oxy-2-methoxyimino-N,3-dimethyl-pent-3-enamide (A.1.34), (Z,2E)-5-[1-(4-chlorophenyl)pyrazol-3-yl]oxy-2-methoxyimino-N,3-dimethyl-pent-3-enamide (A.1.35), pyriminostrobin (A.1.36), bifujunzhi (A.1.37), 2-(ortho-((2,5-dimethylphenyl-oxymethylen)phenyl)-3-methoxy-acrylic acid methylester (A.1.38);
Inhibitors of complex III at Qᵢ site: cyazofamid (A.2.1), amisulbrom (A.2.2), [(6S,7R,8R)-8-benzyl-3-[(3-hydroxy-4-methoxy-pyridine-2-carbonyl)amino]-6-methyl-4,9-dioxo-1,5-dioxonan-7-yl] 2-methylpropanoate (A.2.3), fenpicoxamid (A.2.4), [(6S,7R,8R)-8-benzyl-3-[[4-methoxy-3-(propanoyloxymethoxy)pyridine-2-carbonyl]amino]-6-methyl-4,9-dioxo-1,5-dioxonan-7-yl] 2-methylpropanoate (A.2.5), florylpicoxamid (A.2.6);
Inhibitors of complex II benodanil (A.3.1), benzovindiflupyr (A.3.2), bixafen (A.3.3), boscalid (A.3.4), carboxin (A.3.5), fenfuram (A.3.6), fluopyram (A.3.7), flutolanil (A.3.8), fluxapyroxad (A.3.9), furametpyr (A.3.10), isofetamid (A.3.11), isopyrazam (A.3.12), mepronil (A.3.13), oxycarboxin (A.3.14), penflufen (A.3.15), penthiopyrad (A.3.16), pydiflumetofen (A.3.17), N-[2-(3,4-difluorophenyl)phenyl]-3-(trifluoromethyl)pyrazine-2-carboxamide (A.3.18), sedaxane (A.3.19), tecloftalam (A.3.20), thifluzamide (A.3.21), 3-(difluoromethyl)-1-methyl-N-(1,1,3-trimethyl-indan-4-yl)pyrazole-4-carboxamide (A.3.22), 3-(trifluoromethyl)-1-methyl-N-(1,1,3-trimethylindan-4-yl)pyrazole-4-carboxamide (A.3.23), 1,3-dimethyl-N-(1,1,3-trimethylindan-4-yl)pyrazole-4-carboxamide (A.3.24), 3-(trifluoromethyl)-1,5-dimethyl-N-(1,1,3-trimethylindan-4-yl)pyrazole-4-carboxamide (A.3.25), 1,3,5-trimethyl-N-(1,1,3-trimethylindan-4-yl)pyrazole-4-carboxamide (A.3.26), 3-(difluoromethyl)-1,5-dimethyl-N-(1,1,3-trimethylindan-4-yl)pyrazole-4-carboxamide (A.3.27), 3-(difluoromethyl)-N-(7-fluoro-1,1,3-trimethyl-indan-4-yl)-1-methyl-pyrazole-4-carboxamide (A.3.28), N-[2-[2-chloro-4-(trifluoromethyl)phenoxy]phenyl]-3-(difluoromethyl)-5-fluoro-1-methyl-pyrazole-4-carboxamide (A.3.29), methyl (E)-2-[2-[(5-cyano-2-methyl-phenoxy)methyl]phenyl]-3-methoxy-prop-2-enoate (A.3.30), N-[(5-chloro-2-isopropyl-phenyl)methyl]-N-cyclopropyl-3-(difluoromethyl)-5 fluoro-1-methyl-pyrazole-4-carboxamide (A.3.31), 2-(difluoromethyl)-N-(1,1,3-trimethyl-indan-4-yl)pyridine-3-carboxamide (A.3.32), 2-(difluoromethyl)-N-[(3R)-1,1,3-trimethylindan-4-yl]pyridine-3-carboxamide (A.3.33), 2-(difluoromethyl)-N-(3-ethyl-1,1-dimethyl-indan-4-yl)pyridine-3-carboxamide (A.3.34), 2-(difluoromethyl)-N-[(3R)-3-ethyl-1,1-dimethyl-indan-4-yl]pyridine-3-carboxamide (A.3.35), 2-(difluoromethyl)-N-(1,1-dimethyl-3-propyl-indan-4-yl)pyridine-3-carboxamide (A.3.36), 2-(difluoromethyl)-N-[(3R)-1,1-dimethyl-3-propyl-indan-4-yl]pyridine-3-carboxamide (A.3.37), 2-(difluoromethyl)-N-(3-isobutyl-1,1-dimethyl-indan-4-yl)pyridine-3-carboxamide (A.3.38), 2-(difluoromethyl)-N-[(3R)-3-isobutyl-1,1-dimethyl-indan-4 yl]pyridine-3-carboxamide (A.3.39);
Other respiration inhibitors: diflumetorim (A.4.1); nitrophenyl derivates: binapacryl (A.4.2), dinobuton (A.4.3), dinocap (A.4.4), fluazinam (A.4.5), meptyldinocap (A.4.6), ferimzone (A.4.7); organometal compounds: fentin salts, e. g. fentin-acetate (A.4.8), fentin chloride (A.4.9) or fentin hydroxide (A.4.10); ametoctradin (A.4.11); silthiofam (A.4.12);
B) Sterol biosynthesis inhibitors (SBI fungicides)
C14 demethylase inhibitors: triazoles: azaconazole (B.1.1), bitertanol (B.1.2), bromuconazole (B.1.3), cyproconazole (B.1.4), difenoconazole (B.1.5), diniconazole (B.1.6), diniconazole-M (B.1.7), epoxiconazole (B.1.8), fenbuconazole (B.1.9), fluquinconazole (B.1.10), flusilazole (B.1.11), flutriafol (B.1.12), hexaconazole (B.1.13), imibenconazole (B.1.14), ipconazole (B.1.15), metconazole (B.1.17), myclobutanil (B.1.18), oxpoconazole (B.1.19), paclobutrazole (B.1.20), penconazole (B.1.21), propiconazole (B.1.22), prothioconazole (B.1.23), simeconazole (B.1.24), tebuconazole (B.1.25), tetraconazole (B.1.26), triadimefon (B.1.27), triadimenol (B.1.28), triticonazole (B.1.29), uniconazole (B.1.30), 1-[rel-(2S;3R)-3-(2-chloro-phenyl)-2-(2,4-difluorophenyl)-oxiranylmethyl]-5-thiocyanato-1H-[1,2,4]triazole (B.1.31), 2-[rel-(2S;3R)-3-(2-chlorophenyl)-2-(2,4-difluorophenyl)-oxiranylmethyl]-2H-[1,2,4]triazole-3-thiol (B.1.32), 2-[2-chloro-4-(4-chlorophenoxy)phenyl]-1-(1,2,4-triazol-1-yl)pentan-2-ol (B.1.33), 1-[4-(4-chlorophenoxy)-2-(trifluoro-methyl)phenyl]-1-cyclopropyl-2-(1,2,4-triazol-1-yl)ethanol (B.1.34), 2-[4-(4-chloro-phenoxy)-2-(trifluoromethyl)phenyl]-1-(1,2,4-triazol-1-yl)butan-2-ol (B.1.35), 2-[2-chloro-4-(4-chlorophenoxy)phenyl]-1-(1,2,4-triazol-1-yl)butan-2-ol (B.1.36), ipfentrifluconazole (B.1.37), mefentrifluconazole (B.1.38), 2-[2-chloro-4-(4-chlorophenoxy)phenyl]-3-methyl-1-(1,2,4-triazol-1-yl)butan-2-ol (B.1.39), 2-[4-(4-chlorophenoxy)-2-(trifluoromethyl)phenyl]-1-(1,2,4-triazol-1-yl)pentan-2-ol (B.1.40), 2-[4-(4-fluorophenoxy)-2-(trifluoromethyl)phenyl]-1-(1,2,4-triazol-1-yl)propan-2-ol (B.1.41), 2-[2-chloro-4-(4-chlorophenoxy)phenyl]-1-(1,2,4-triazol-1-yl)pent-3-yn-2-ol (B.1.42), 2-(chloromethyl)-2-methyl-5-(p-tolylmethyl)-1-(1,2,4-triazol-1-ylmethyl)cyclopentanol (B.1.43); imidazoles: imazalil (B.1.44), pefurazoate (B.1.45), prochloraz (B.1.46), triflumizol (B.1.47); pyrimidines, pyridines and piperazines: fenarimol (B.1.49), pyrifenox (B.1.50), triforine (B.1.51), [3-(4-chloro-2-fluoro-phenyl)-5-(2,4-difluorophenyl)isoxazol-4-yl]-(3-pyridyl)methanol (B.1.52);
Delta14-reductase inhibitors: aldimorph (B.2.1), dodemorph (B.2.2), dodemorph-acetate (B.2.3), fenpropimorph (B.2.4), tridemorph (B.2.5), fenpropidin (B.2.6), piperalin (B.2.7), spiroxamine (B.2.8);
Inhibitors of 3-keto reductase: fenhexamid (B.3.1);
Other Sterol biosynthesis inhibitors: chlorphenomizole (B.4.1);
C) Nucleic acid synthesis inhibitors
Phenylamides or acyl amino acid fungicides: benalaxyl (C.1.1), benalaxyl-M (C.1.2), kiralaxyl (C.1.3), metalaxyl (C.1.4), metalaxyl-M (C.1.5), ofurace (C.1.6), oxadixyl (C.1.7);
Other nucleic acid synthesis inhibitors: hymexazole (C.2.1), octhilinone (C.2.2), oxolinic acid (C.2.3), bupirimate (C.2.4), 5-fluorocytosine (C.2.5), 5-fluoro-2-(p-tolylmethoxy)pyrimidin-4-amine (C.2.6), 5-fluoro-2-(4-fluorophenylmethoxy)pyrimidin-4-amine (C.2.7), 5-fluoro-2-(4-chlorophenylmethoxy)pyrimidin-4 amine (C.2.8);
D) Inhibitors of cell division and cytoskeleton
Tubulin inhibitors: benomyl (D.1.1), carbendazim (D.1.2), fuberidazole (D1.3), thiabendazole (D.1.4), thiophanate-methyl (D.1.5), 3-chloro-4-(2,6-difluorophenyl)-6-methyl-5-phenyl-pyridazine (D.1.6), 3-chloro-6-methyl-5-phenyl-4-(2,4,6-trifluorophenyl)pyridazine (D.1.7), N-ethyl-2-[(3-ethynyl-8-methyl-6-quinolyl)oxy]butanamide (D.1.8), N-ethyl-2-[(3-ethynyl-8-methyl-6-quinolyl)oxy]-2-methylsulfanyl-acetamide (D.1.9), 2-[(3-ethynyl-8-methyl-6-quinolyl)oxy]-N-(2-fluoroethyl)butanamide (D.1.10), 2-[(3-ethynyl-8-methyl-6-quinolyl)oxy]-N-(2-flu-oroethyl)-2-methoxy-acetamide (D.1.11), 2-[(3-ethynyl-8-methyl-6-quinolyl)oxy]-N-propyl-butanamide (D.1.12), 2-[(3-ethynyl-8-methyl-6-quinolyl)oxy]-2-methoxy-N-propyl-acetamide (D.1.13), 2-[(3-ethynyl-8-methyl-6-quinolyl)oxy]-2-methylsulfanyl-N-propyl-acetamide (D.1.14), 2-[(3-ethynyl-8-methyl-6-quinolyl)oxy]-N-(2-fluoroethyl)-2-methylsulfanyl-acetamide (D.1.15), 4-(2-bromo-4-fluoro-phenyl)-N-(2-chloro-6-fluoro-phenyl)-2,5-dimethyl-pyrazol-3-amine (D.1.16);
Other cell division inhibitors: diethofencarb (D.2.1), ethaboxam (D.2.2), pencycuron (D.2.3), fluopicolide (D.2.4), zoxamide (D.2.5), metrafenone (D.2.6), pyriofenone (D.2.7).
E) Inhibitors of amino acid and protein synthesis
Methionine synthesis inhibitors: cyprodinil (E.1.1), mepanipyrim (E.1.2), pyrimethanil (E.1.3);
Protein synthesis inhibitors: blasticidin-S(E.2.1), kasugamycin (E.2.2), kasugamycin hydrochloride-hydrate (E.2.3), mildiomycin (E.2.4), streptomycin (E.2.5), oxytetracyclin (E.2.6);
F) Signal transduction inhibitors
MAP/histidine kinase inhibitors: fluoroimid (F.1.1), iprodione (F.1.2), procymidone (F.1.3), vinclozolin (F.1.4), fludioxonil (F.1.5);
G protein inhibitors: quinoxyfen (F.2.1);
G) Lipid and membrane synthesis inhibitors
Phospholipid biosynthesis inhibitors: edifenphos (G.1.1), iprobenfos (G.1.2), pyrazophos (G.1.3), isoprothiolane (G.1.4);
Lipid peroxidation: dicloran (G.2.1), quintozene (G.2.2), tecnazene (G.2.3), tolclofos-methyl (G.2.4), biphenyl (G.2.5), chloroneb (G.2.6), etridiazole (G.2.7);
Phospholipid biosynthesis and cell wall deposition: dimethomorph (G.3.1), flumorph (G.3.2), mandipropamid (G.3.3), pyrimorph (G.3.4), benthiavalicarb (G.3.5), iprovalicarb (G.3.6), valifenalate (G.3.7);
Compounds affecting cell membrane permeability and fatty acides: propamocarb (G.4.1);
Inhibitors of oxysterol binding protein: oxathiapiprolin (G.5.1), 2-{3-[2-(1-{[3,5-bis(difluoromethyl-1H-pyrazol-1-yl]acetyl}piperidin-4-yl)-1,3-thiazol-4-yl]-4,5-dihydro-1,2-oxazol-5-yl}phenyl methanesulfonate (G.5.2), 2-{3-[2-(1-{[3,5-bis(difluoromethyl)-1H-pyrazol-1-yl]acetyl}piperidin-4-yl) 1,3-thiazol-4-yl]-4,5-dihydro-1,2-oxazol-5-yl}-3-chlorophenyl methanesulfonate (G.5.3), 4-[1-[2-[3-(difluoromethyl)-5-methyl-pyrazol-1-yl]acetyl]-4-piperidyl]-N-tetralin-1-yl-pyridine-2-carboxamide (G.5.4), 4-[1-[2-[3,5-bis(difluoromethyl)pyrazol-1-yl]acetyl]-4-piperidyl]-N-tetralin-1-yl-pyridine-2-carboxamide (G.5.5), 4-[1-[2-[3-(difluoromethyl)-5-(trifluoromethyl)pyrazol-1-yl]acetyl]-4-piperidyl]-N-tetralin-1-yl-pyridine-2-carboxamide (G.5.6), 4-[1-[2-[5-cyclopropyl-3-(difluoromethyl)pyrazol-1-yl]acetyl]-4-piperidyl]-N-tetralin-1-yl-pyridine-2-carboxamide (G.5.7), 4-[1-[2-[5-methyl-3-(trifluoromethyl)pyrazol-1-yl]acetyl]-4-piperidyl]-N-tetralin-1-yl-pyridine-2-carboxamide (G.5.8), 4-[1-[2-[5-(difluoromethyl)-3-(trifluoromethyl)pyrazol-1-yl]acetyl]-4-piperidyl]-N-tetralin-1-yl-pyridine-2-carboxamide (G.5.9), 4-[1-[2-[3,5-bis(trifluoromethyl)pyrazol-1-yl]acetyl]-4-piperidyl]-N-tetralin-1-yl-pyridine-2-carboxamide (G.5.10), (4-[1-[2-[5-cyclopropyl-3-(trifluoromethyl)pyrazol-1-yl]acetyl]-4-piperidyl]-N-tetralin-1-yl-pyridine-2-carboxamide (G.5.11);
H) Inhibitors with Multi Site Action
Inorganic active substances: Bordeaux mixture (H.1.1), copper (H.1.2), copper acetate (H.1.3), copper hydroxide (H.1.4), copper oxychloride (H.1.5), basic copper sulfate (H.1.6), sulfur (H.1.7);
Thio- and dithiocarbamates: ferbam (H.2.1), mancozeb (H.2.2), maneb (H.2.3), metam (H.2.4), metiram (H.2.5), propineb (H.2.6), thiram (H.2.7), zineb (H.2.8), ziram (H.2.9);
Organochlorine compounds: anilazine (H.3.1), chlorothalonil (H.3.2), captafol (H.3.3), captan (H.3.4), folpet (H.3.5), dichlofluanid (H.3.6), dichlorophen (H.3.7), hexachlorobenzene (H.3.8), pentachlorphenole (H.3.9) and its salts, phthalide (H.3.10), tolylfluanid (H.3.11);
Guanidines and others: guanidine (H.4.1), dodine (H.4.2), dodine free base (H.4.3), guazatine (H.4.4), guazatine-acetate (H.4.5), iminoctadine (H.4.6), iminoctadine-triacetate (H.4.7), iminoctadine-tris(albesilate) (H.4.8), dithianon (H.4.9), 2,6-di-methyl-1H,5H-[1,4]dithiino[2,3-c:5,6-c′]dipyrrole-1,3,5,7(2H,6H)-tetraone (H.4.10);
I) Cell wall synthesis inhibitors
Inhibitors of glucan synthesis: validamycin (I.1.1), polyoxin B (I.1.2);
Melanin synthesis inhibitors: pyroquilon (I.2.1), tricyclazole (I.2.2), carpropamid (I.2.3), dicyclomet (I.2.4), fenoxanil (I.2.5);
J) Plant defence inducers
Acibenzolar-S-methyl (J.1.1), probenazole (J.1.2), isotianil (J.1.3), tiadinil (J.1.4), prohexadione-calcium (J.1.5); phosphonates: fosetyl (J.1.6), fosetyl-aluminum (J.1.7), phosphorous acid and its salts (J.1.8), potassium or sodium bicarbonate (J.1.9), 4-cyclopropyl-N-(2,4-dimethoxyphenyl)thiadiazole-5-carboxamide (J.1.10), calcium phosphonate (J.1.11), potassium phosphonate (J.1.12);
K) Unknown mode of action
Bronopol (K.1.1), chinomethionat (K.1.2), cyflufenamid (K.1.3), cymoxanil (K.1.4), dazomet (K.1.5), debacarb (K.1.6), diclocymet (K.1.7), diclomezine (K.1.8), difenzoquat (K.1.9), difenzoquat-methylsulfate (K.1.10), diphenylamin (K.1.11), fenitropan (K.1.12), fenpyrazamine (K.1.13), flumetover (K.1.14), flusulfamide (K.1.15), flutianil (K.1.16), harpin (K.1.17), methasulfocarb (K.1.18), nitrapyrin (K.1.19), nitrothal-isopropyl (K.1.20), tolprocarb (K.1.21), oxin-copper (K.1.22), proquinazid (K.1.23), tebufloquin (K.1.24), tecloftalam (K.1.25), triazoxide (K.1.26), N′-(4-(4-chloro-3-trifluoromethyl-phenoxy)-2,5-dimethyl-phenyl)-N-ethyl-N-methyl formamidine (K.1.27), N′-(4-(4-fluoro-3-trifluoromethyl-phenoxy)-2,5-dimethyl-phenyl)-N-ethyl-N-methyl formamidine (K.1.28), N′-[4-[[3-[(4-chlorophenyl)methyl]-1,2,4-thiadiazol-5-yl]oxy]-2,5-dimethyl-phenyl]-N-ethyl-N-methyl-formamidine (K.1.29), N′-(5-bromo-6-indan-2-yloxy-2-methyl-3-pyridyl)-N-ethyl-N-methyl-formamidine (K.1.30), N′-[5-bromo-6-[1-(3,5-difluorophenyl)ethoxy]-2-methyl-3-pyridyl]-N-ethyl-N-methyl-formamidine (K.1.31), N′-[5-bromo-6-(4-isopropylcyclohexoxy)-2-methyl-3-pyridyl]-N-ethyl-N-methyl-formamidine (K.1.32), N′-[5-bromo-2-methyl-6-(1-phenylethoxy)-3-pyridyl]-N-ethyl-N-methyl-formamidine (K.1.33), N′-(2-methyl-5-trifluoromethyl-4-(3-trimethylsilanyl-propoxy)-phenyl)-N-ethyl-N-methyl formamidine (K.1.34), N′-(5-difluoromethyl-2-methyl-4-(3-trimethylsilanyl-propoxy)-phenyl)-N-ethyl-N-methyl formamidine (K.1.35), 2-(4-chloro-phenyl)-N-[4-(3,4-dimethoxy-phenyl)-isoxazol-5-yl]-2-prop-2-ynyloxy-acetamide (K.1.36), 3-[5-(4-chloro-phenyl)-2,3-dimethyl-isoxazolidin-3-yl]-pyridine (pyrisoxazole) (K.1.37), 3-[5-(4-methylphenyl)-2,3-dimethyl-isoxazolidin-3 yl]-pyridine (K.1.38), 5-chloro-1-(4,6-dimethoxy-pyrimidin-2-yl)-2-methyl-1H-benzoimidazole (K.1.39), ethyl (Z)-3-amino-2-cyano-3-phenyl-prop-2-enoate (K.1.40), picarbutrazox (K.1.41), pentyl N-[6-[[(Z)-[(1-methyltetrazol-5-yl)-phenyl-methylene]amino]oxymethyl]-2-pyridyl]carbamate (K.1.42), but-3-ynyl N-[6-[[(Z)-[(1-methyltetrazol-5-yl)-phenyl-methylene]amino]oxymethyl]-2-pyridyl]carbamate (K.1.43), 2-[2-[(7,8-difluoro-2-methyl-3-quinolyl)oxy]-6-fluoro-phenyl]propan-2-ol (K.1.44), 2-[2-fluoro-6-[(8-fluoro-2-methyl-3-quinolyl)oxy]phen-yl]propan-2-ol (K.1.45), 3-(5-fluoro-3,3,4,4-tetramethyl-3,4-dihydroisoquinolin-1-yl)quinoline (K.1.46), quinofumelin (K.1.47), 3-(4,4,5-trifluoro-3,3-dimethyl-3,4-dihydroisoquinolin-1-yl)quinoline (K.1.48), 9-fluoro-2,2-dimethyl-5-(3-quinolyl)-3H-1,4-benzoxazepine (K.1.49), 2-(6-benzyl-2-pyridyl)quinazoline (K.1.50), 2-[6-(3-fluoro-4-methoxy-phenyl)-5-methyl-2-pyridyl]quinazoline (K.1.51), dichlobentiazox (K.1.52), N′-(2,5-dimethyl-4-phenoxy-phenyl)-N-ethyl-N-methyl-formamidine (K.1.53), N′-[5-bromo-2-methyl-6-(1-methyl-2-propoxy-ethoxy)-3-pyridyl]-N-ethyl-N-methyl-formamidine (K.1.54);
M) Growth regulators
abscisic acid (M.1.1), amidochlor, ancymidol, 6-benzylaminopurine, brassinolide, butralin, chlormequat, chlormequat chloride, choline chloride, cyclanilide, daminozide, dikegulac, dimethipin, 2,6-dimethylpuridine, ethephon, flumetralin, flurprimidol, fluthiacet, forchlorfenuron, gibberellic acid, inabenfide, indole-3-acetic acid, maleic hydrazide, mefluidide, mepiquat, mepiquat chloride, naphthaleneacetic acid, N-6-benzyladenine, paclobutrazol, prohexadione, prohexadione-calcium, prohydrojasmon, thidiazuron, triapenthenol, tributyl phosphorotrithioate, 2,3,5-tri-iodobenzoic acid, trinexapac-ethyl and uniconazole;
N) Herbicides from classes N.1 to N.15
N.1 Lipid biosynthesis inhibitors: alloxydim (N.1.1), alloxydim-sodium (N.1.2), butroxydim (N.1.3), clethodim (N.1.4), clodinafop (N.1.5), clodinafop-propargyl (N.1.6), cycloxydim (N.1.7), cyhalofop (N.1.8), cyhalofop-butyl (N.1.9), diclofop(N.1.10), diclofop-methyl (N.1.11), fenoxaprop (N.1.12), fenoxaprop-ethyl (N.1.13), fenoxaprop-P (N.1.14), fenoxaprop-P-ethyl (N.1.15), fluazifop (N.1.16), fluazifop-butyl (N.1.17), fluazifop-P (N.1.18), fluazifop-P-butyl (N.1.19), haloxyfop (N.1.20), haloxyfop-methyl (N.1.21), haloxyfop-P (N.1.22), haloxyfop-P-methyl (N.1.23), metamifop (N.1.24), pinoxaden (N.1.25), profoxydim (N.1.26), propaquizafop (N.1.27), quizalofop (N.1.28), quizalofop-ethyl (N.1.29), quizalofop-tefuryl (N.1.30), quizalofop-P (N.1.31), quizalofop-P-ethyl (N.1.32), quizalofop-P-tefuryl (N.1.33), sethoxydim (N.1.34), tepraloxydim (N.1.35), tralkoxydim (N.1.36), 4-(4′-chloro-4-cyclo¬propyl-2′-fluoro[1,1′-biphenyl]-3-y)-5-hydroxy-2,2,6,6-tetramethyl-2H-pyran-3(6H)-one ((N.1.37) CAS 1312337-72-6); 4-(2′,4′-dichloro-4-cyclopropyl[1,1′-biphenyl]-3-yl)-5-hydroxy-2,2,6,6-tetramethyl-2H-pyran-3(6H)-one ((N.1.38) CAS 1312337-45-3); 4-(4′-chloro-4-ethyl-2′-fluoro[1,1′-biphenyl]-3-y)-5-hydroxy-2,2,6,6-tetramethyl-2H-pyran-3(6H)-one ((N.1.39) CAS 1033757-93-5); 4-(2′,4′-Dichloro-4-ethyl[1,1′-biphenyl]-3-yl)-2,2,6,6-tetramethyl-2H-pyran-3,5(4H,6H)-dione ((N.1.40) CAS 1312340-84-3); 5-(acetyloxy)-4-(4′-chloro-4-cyclopropyl-2′-fluoro[1,1′-biphenyl]-3-yl)-3,6-dihydro-2,2,6,6-tetramethyl-2H-pyran-3-one ((N.1.41) CAS 1312337-48-6); 5-(acetyloxy)-4-(2′,4′-dichloro-4-cyclopropyl-[1,1′-biphenyl]-3-yl)-3,6-dihydro-2,2,6,6-tetramethyl-2H-pyran-3-one (N.1.42); 5-(acetyloxy)-4-(4′-chloro-4-ethyl-2′-fluoro[1,1′-biphenyl]-3-yl)-3,6-dihydro-2,2,6,6-tetramethyl-2H-pyran-3-one ((N.1.43) CAS 1312340-82-1); 5-(acetyloxy)-4-(2′,4′-dichloro-4-ethyl[1,1′-biphenyl]-3-yl)-3,6-dihydro-2,2,6,6-tetramethyl-2H-pyran-3-one ((N.1.44) CAS 1033760-55-2); 4-(4′-chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester ((N.1.45) CAS 1312337-51-1); 4-(2′,4′-dichloro-4-cyclopropyl-[1,1′-biphenyl]-3-y)-5,6-dihydro-2,2,6,6-tetramethyl-5-oxo-2H-pyran-3-yl carbonic acid methyl ester (N.1.46); 4-(4′-chloro-4-ethyl-2′-fluoro[1,1′-biphenyl]-3-yl)-5,6-dihydro-2,2,6,6-tetramethyl-5-oxo-2H-pyran-3-yl carbonic acid methyl ester ((N.1.47) CAS 1312340-83-2); 4-(2′,4′-dichloro-4-ethyl,[1,1′-biphenyl]-3-y)-5,6-dihydro-2,2,6,6-tetramethyl-5-oxo-2H-pyran-3-yl carbonic acid methyl ester ((N.1.48) CAS 1033760-58-5); benfuresate (N.1.49), butylate (N.1.50), cycloate (N.1.51), dalapon (N.1.52), dimepiperate (N.1.53), EPTC (N.1.54), esprocarb (N.1.55), ethofumesate (N.1.56), flupropanate (N.1.57), molinate (N.1.58), orbencarb (N.1.59), pebulate (N.1.60), prosulfocarb (N.1.61), TCA (N.1.62), thiobencarb (N.1.63), tiocarbazil (N.1.64), triallate (N.1.65) and vernolate (N.1.66);
N.2 ALS inhibitors: amidosulfuron (N.2.1), azimsulfuron (N.2.2), bensulfuron (N.2.3), bensulfuron-methyl (N.2.4), chlorimuron (N.2.5), chlorimuron-ethyl (N.2.6), chlorsulfuron (N.2.7), cinosulfuron (N.2.8), cyclosulfamuron (N.2.9), ethametsulfuron (N.2.10), ethametsulfuron-methyl (N.2.11), ethoxysulfuron (N.2.12), flazasulfuron (N.2.13), flucetosulfuron (N.2.14), flupyrsulfuron (N.2.15), flupyrsulfuron-methyl-sodium (N.2.16), foramsulfuron (N.2.17), halosulfuron (N.2.18), halosulfuron-methyl (N.2.19), imazosulfuron (N.2.20), iodosulfuron (N.2.21), iodosulfuron-methyl-sodium (N.2.22), iofensulfuron (N.2.23), iofensulfuron-sodium (N.2.24), mesosulfuron (N.2.25), metazosulfuron (N.2.26), metsulfuron (N.2.27), metsulfuron-methyl (N.2.28), nicosulfuron (N.2.29), orthosulfamuron (N.2.30), oxasulfuron (N.2.31), primisulfuron (N.2.32), primisulfuron-methyl (N.2.33), propyrisulfuron (N.2.34), prosulfuron (N.2.35), pyrazosulfuron (N.2.36), pyrazosulfuron-ethyl (N.2.37), rimsulfuron (N.2.38), sulfometuron (N.2.39), sulfometuron-methyl (N.2.40), sulfosulfuron (N.2.41), thifensulfuron (N.2.42), thifensulfuron-methyl (N.2.43), triasulfuron (N.2.44), tribenuron (N.2.45), tribenuron-methyl (N.2.46), trifloxysulfuron (N.2.47), triflusulfuron (N.2.48), triflusulfuron-methyl (N.2.49), tritosulfuron (N.2.50), imazamethabenz (N.2.51), imazamethabenz-methyl (N.2.52), imazamox (N.2.53), imazapic (N.2.54), imazapyr (N.2.55), imazaquin (N.2.56), imazethapyr (N.2.57); cloransulam (N.2.58), cloransulam-methyl (N.2.59), diclosulam (N.2.60), flumetsulam (N.2.61), florasulam (N.2.62), metosulam (N.2.63), penoxsulam (N.2.64), pyrimisulfan (N.2.65) and pyroxsulam (N.2.66); bispyribac (N.2.67), bispyribac-sodium (N.2.68), pyribenzoxim (N.2.69), pyriftalid (N.2.70), pyriminobac (N.2.71), pyriminobac-methyl (N.2.72), pyrithiobac (N.2.73), pyrithiobac-sodium (N.2.74), 4-[[[2-[(4,6-dimethoxy-2-pyrimidinyl)oxy]phenyl]methyl]amino]-benzoic acid-1-methyl¬ethyl ester ((N.2.75) CAS 420138-41-6), 4-[[[2-[(4,6-dimethoxy-2-pyrimidinyl)oxy]phenyl]¬methyl]amino]-benzoic acid propyl ester ((N.2.76) CAS 420138-40-5), N-(4-bromophenyl)-2-[(4,6-dimethoxy-2-pyrimidinyl)oxy]-benzenemethanamine ((N.2.77) CAS 420138-01-8); flucarbazone (N.2.78), flucarbazone-sodium (N.2.79), propoxycarbazone (N.2.80), propoxycarbazone-sodium (N.2.81), thiencarbazone (N.2.82), thiencarbazone-methyl (N.2.83), triafamone (N.2.84);
N.3 Photosynthesis inhibitors: amicarbazone (N.3.1); chlorotriazine (N.3.2); ametryn (N.3.3), atrazine (N.3.4), chloridazone (N.3.5), cyanazine (N.3.6), desmetryn (N.3.7), dimethametryn (N.3.8),hexazinone (N.3.9), metribuzin (N.3.10), prometon (N.3.11), prometryn (N.3.12), propazine (N.3.13), simazine (N.3.14), simetryn (N.3.15), terbumeton (N.3.16), terbuthylazin (N.3.17), terbutryn (N.3.18), trietazin (N.3.19); chlorobromuron (N.3.20), chlorotoluron (N.3.21), chloroxuron (N.3.22), dimefuron (N.3.23), diuron (N.3.24), fluometuron (N.3.25), isoproturon (N.3.26), isouron (N.3.27), linuron (N.3.28), metamitron (N.3.29), methabenzthiazuron (N.3.30), metobenzuron (N.3.31), metoxuron (N.3.32), monolinuron (N.3.33), neburon (N.3.34), siduron (N.3.35), tebuthiuron (N.3.36), thiadiazuron (N.3.37), desmedipham (N.3.38), karbutilat (N.3.39), phenmedipham (N.3.40), phenmedipham-ethyl (N.3.41), bromofenoxim (N.3.42), bromoxynil (N.3.43) and its salts and esters, ioxynil (N.3.44) and its salts and esters, bromacil (N.3.45), lenacil (N.3.46), terbacil (N.3.47), bentazon (N.3.48), bentazon-sodium (N.3.49), pyridate (N.3.50), pyridafol (N.3.51), pentanochlor (N.3.52), propanil (N.3.53); diquat (N.3.54), diquat-dibromide (N.3.55), paraquat (N.3.56), paraquat-dichloride (N.3.57), paraquat-dimetilsulfate (N.3.58);
N.4 protoporphyrinogen-IX oxidase inhibitors: acifluorfen (N.4.1), acifluorfen-sodium (N.4.2), azafenidin (N.4.3), bencarbazone (N.4.4), benzfendizone (N.4.5), bifenox (N.4.6), butafenacil (N.4.7), carfentrazone (N.4.8), carfentrazone-ethyl (N.4.9), chlormethoxyfen (N.4.10), cinidon-ethyl (N.4.11), fluazolate (N.4.12), flufenpyr (N.4.13), flufenpyr-ethyl (N.4.14), flumiclorac (N.4.15), flumiclorac-pentyl (N.4.16), flumioxazin (N.4.17), fluoroglycofen (N.4.18), fluoroglycofen-ethyl (N.4.19), fluthiacet (N.4.20), fluthiacet-methyl (N.4.21), fomesafen (N.4.22), halosafen (N.4.23), lactofen (N.4.24), oxadiargyl (N.4.25), oxadiazon (N.4.26), oxyfluorfen (N.4.27), pentoxazone (N.4.28), profluazol (N.4.29), pyraclonil (N.4.30), pyraflufen (N.4.31), pyraflufen-ethyl (N.4.32), saflufenacil (N.4.33), sulfentrazone (N.4.34), thidiazimin (N.4.35), tiafenacil (N.4.36), trifludimoxazin (N.4.37), ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate ((N.4.38) CAS 353292-31-6), N-ethyl-3-(2,6-dichloro-4-trifluoro-methylphenoxy)-5-methyl-1H-pyrazole-1-carboxamide ((N.4.39) CAS 452098-92-9), N tetrahydrofurfuryl-3-(2,6-dichloro-4-trifluoromethylphenoxy)-5-methyl-1H-pyrazole-1-carboxamide ((N.4.40) CAS 915396-43-9), N-ethyl-3-(2-chloro-6-fluoro-4-trifluoromethyl¬phenoxy)-5-methyl-1H-pyrazole-1-carboxamide ((N.4.41) CAS 452099-05-7), N tetrahydro¬furfuryl-3-(2-chloro-6-fluoro-4-trifluoro-methylphenoxy)-5-methyl-1H-pyrazole-1-carboxamide ((N.4.42) CAS 452100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione ((N.4.43) CAS 451484-50-7), 2-(2,2,7-trifluoro-3-oxo-4-prop-2-ynyl-3,4-dihydro-2H-benzo[1,4]oxazin-6-yl)-4,5,6,7-tetrahydro-isoindole-1,3-dione ((N.4.44) CAS 1300118-96-0), 1-methyl-6-trifluoro¬methyl-3-(2,2,7-tri-fluoro-3-oxo-4-prop-2-ynyl-3,4-dihydro-2H-benzo[1,4]oxazin-6-yl)-1H-pyrimidine-2,4-dione ((N.4.45) CAS 1304113-05-0), methyl (E)-4-[2-chloro-5-[4-chloro-5-(difluoromethoxy)-1H-methyl-pyrazol-3-yl]-4-fluoro-phenoxy]-3-methoxy-but-2-enoate ((N.4.46) CAS 948893-00-3), 3-[7-chloro-5-fluoro-2-(trifluoromethyl)-1H-benzimidazol-4-yl]-1-methyl-6-(trifluoromethyl)-1H-pyrimidine-2,4-dione ((N.4.47) CAS 212754-02-4);
N.5 Bleacher herbicides: beflubutamid (N.5.1), diflufenican (N.5.2), fluridone (N.5.3), flurochloridone (N.5.4), flurtamone (N.5.5), norflurazon (N.5.6), picolinafen (N.5.7), 4-(3-trifluoromethyl¬phenoxy)-2-(4-trifluoromethylphenyl),pyrimidine ((N.5.8) CAS 180608-33-7); benzobicyclon (N.5.9), benzofenap (N.5.10), bicyclopyrone (N.5.11), clomazone (N.5.12), fenquintrione (N.5.13), isoxaflutole (N.5.14), mesotrione (N.5.15), pyrasulfotole (N.5.16), pyrazolynate (N.5.17), pyrazoxyfen (N.5.18), sulcotrione (N.5.19), tefuryltrione (N.5.20), tembotrione (N.5.21), tolpyralate (N.5.22), topramezone (N.5.23); aclonifen (N.5.24), amitrole (N.5.25), flumeturon (N.5.26);
N.6 EPSP synthase inhibitors: glyphosate (N.6.1), glyphosate-isopropylammonium (N.6.2), glyposate-potassium (N.6.3), glyphosate-trimesium (sulfosate) (N.6.4);
N.7 Glutamine synthase inhibitors: bilanaphos (bialaphos) (N.7.1), bilanaphos-sodium (N.7.2), glufosinate (N.7.3), glufosinate-P (N.7.4), glufosinate-ammonium (N.7.5);
N.8 DHP synthase inhibitors: asulam (N.8.1);
N.9 Mitosis inhibitors: benfluralin (N.9.1), butralin (N.9.2), dinitramine (N.9.3), ethalfluralin (N.9.4), fluchloralin (N.9.5), oryzalin (N.9.6), pendimethalin (N.9.7), prodiamine (N.9.8), trifluralin (N.9.9); amiprophos (N.9.10), amiprophos-methyl (N.9.11), butamiphos (N.9.12); chlorthal (N.9.13), chlorthal-dimethyl (N.9.14), dithiopyr (N.9.15), thiazopyr (N.9.16), propyzamide (N.9.17), tebutam (N.9.18); carbetamide (N.9.19), chlorpropham (N.9.20), flamprop (N.9.21), flamprop-isopropyl (N.9.22), flamprop-methyl (N.9.23), flamprop-M-isopropyl (N.9.24), flamprop-M-methyl (N.9.25), propham (N.9.26);
N.10 VLCFA inhibitors: acetochlor (N.10.1), alachlor (N.10.2), butachlor (N.10.3), dimethachlor (N.10.4), dimethenamid (N.10.5), dimethenamid-P (N.10.6), metazachlor (N.10.7), metolachlor (N.10.8), metolachlor-S(N.10.9), pethoxamid (N.10.10), pretilachlor (N.10.11), propachlor (N.10.12), propisochlor (N.10.13), thenylchlor (N.10.14), flufenacet (N.10.15), mefenacet (N.10.16), diphenamid (N.10.17), naproanilide (N.10.18), napropamide (N.10.19), napropamide-M (N.10.20), fentrazamide (N.10.21), anilofos (N.10.22), cafenstrole (N.10.23), fenoxasulfone (N.10.24), ipfencarbazone (N.10.25), piperophos (N.10.26), pyroxasulfone (N.10.27), isoxazoline compounds of the formulae II.1, II.2, II.3, II.4, II.5, II.6, II.7, II.8 and II.9 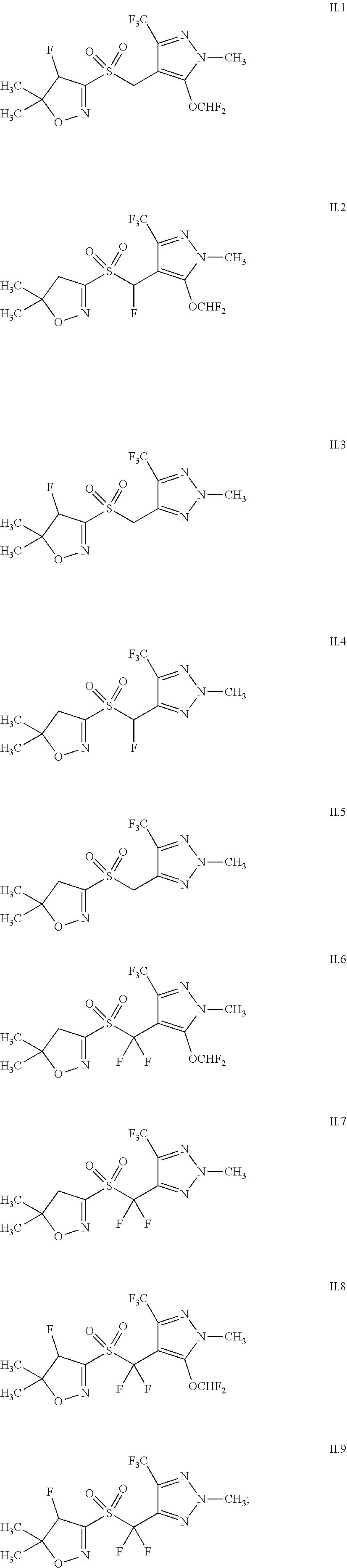
N.11 Cellulose biosynthesis inhibitors: chlorthiamid (N.11.1), dichlobenil (N.11.2), flupoxam (N.11.3), indaziflam (N.11.4), isoxaben (N.11.5), triaziflam (N.11.6), 1-cyclohexyl-5-pentafluorphenyloxy-14-[1,2,4,6]thiatriazin-3-ylamine ((N.11.7) CAS 175899-01-1);
N.12 Decoupler herbicides: dinoseb (N.12.1), dinoterb (N.12.2), DNOC (N.12.3) and its salts;
N.13 Auxinic herbicides: 2,4-D (N.13.1) and its salts and esters, clacyfos (N.13.2), 2,4-DB (N.13.3) and its salts and esters, aminocyclopyrachlor (N.13.4) and its salts and esters, aminopyralid (N.13.5) and its salts such as aminopyralid-dimethylammonium (N.13.6), aminopyralid-tris(2-hydroxypropyl)ammonium (N.13.7) and its esters, benazolin (N.13.8), benazolin-ethyl (N.13.9), chloramben (N.13.10) and its salts and esters, clomeprop (N.13.11), clopyralid (N.13.12) and its salts and esters, dicamba (N.13.13) and its salts and esters, dichlorprop (N.13.14) and its salts and esters, dichlorprop-P (N.13.15) and its salts and esters, fluroxypyr (N.13.16), fluroxypyr-butometyl (N.13.17), fluroxypyr-meptyl (N.13.18), halauxifen (N.13.) and its salts and esters (CAS 943832-60-8); MCPA (N.13.) and its salts and esters, MCPA-thioethyl (N.13.19), MCPB (N.13.20) and its salts and esters, mecoprop (N.13.21) and its salts and esters, mecoprop-P (N.13.22) and its salts and esters, picloram (N.13.23) and its salts and esters, quinclorac (N.13.24), quinmerac (N.13.25), TBA (2,3,6) (N.13.26) and its salts and esters, triclopyr (N.13.27) and its salts and esters, 4-amino-3-chloro-6-(4-chloro-2-fluoro-3-methoxyphenyl)-5-fluoropyridine-2-carboxylic acid (N.13.28), benzyl 4-amino-3-chloro-6-(4-chloro-2-fluoro-3-methoxyphenyl)-5-fluoropyridine-2-carboxylate ((N.13.29) CAS 1390661-72-9);
N.14 Auxin transport inhibitors: diflufenzopyr (N.14.1), diflufenzopyr-sodium (N.14.2), naptalam (N.14.3) and naptalam-sodium (N.14.4);
N.15 Other herbicides: bromobutide (N.15.1), chlorflurenol (N.15.2), chlorflurenol-methyl (N.15.3), cinmethylin (N.15.4), cumyluron (N.15.5), cyclopyrimorate ((N.15.6) CAS 499223-49-3) and its salts and esters, dalapon (N.15.7), dazomet (N.15.8), difenzoquat (N.15.9), difenzoquat-metilsulfate (N.15.10), dimethipin (N.15.11), DSMA (N.15.12), dymron (N.15.13), endothal (N.15.14) and its salts, etobenzanid (N.15.15), flurenol (N.15.16), flurenol-butyl (N.15.17), flurprimidol (N.15.18), fosamine (N.15.19), fosamine-ammonium (N.15.20), indanofan (N.15.21), maleic hydrazide (N.15.22), mefluidide (N.15.23), metam (N.15.24), methiozolin ((N.15.25) CAS 403640-27-7), methyl azide (N.15.26), methyl bromide (N.15.27), methyl-dymron (N.15.28), methyl iodide (N.15.29), MSMA (N.15.30), oleic acid (N.15.31), oxaziclomefone (N.15.32), pelargonic acid (N.15.33), pyributicarb (N.15.34), quinoclamine (N.15.35), tridiphane (N.15.36);
O) Insecticides from classes O.1 to O.29
O.1 Acetylcholine esterase (AChE) inhibitors: aldicarb (O.1.1), alanycarb (O.1.2), bendiocarb (O.1.3), benfuracarb (O.1.4), butocarboxim (O.1.5), butoxycarboxim (O.1.6), carbaryl (O.1.7), carbofuran (O.1.8), carbosulfan (O.1.9), ethiofencarb (O.1.10), fenobucarb (O.1.11), formetanate (O.1.12), furathiocarb (O.1.13), isoprocarb (O.1.14), methiocarb (O.1.15), methomyl (O.1.16), metolcarb (O.1.17), oxamyl (O.1.18), pirimicarb (O.1.19), propoxur (O.1.20), thiodicarb (O.1.21), thiofanox (O.1.22), trimethacarb (O.1.23), XMC (O.1.24), xylylcarb (O.1.25) and triazamate (O.1.26); acephate (O.1.27), azamethiphos (O.1.28), azinphos-ethyl (O.1.29), azinphosmethyl (O.1.30), cadusafos (O.1.31), chlorethoxyfos (O.1.32), chlorfenvinphos (O.1.33), chlormephos (O.1.34), chlorpyrifos (O.1.35), chlorpyrifos-methyl (O.1.36), coumaphos (O.1.37), cyanophos (O.1.38), demeton-S-methyl (O.1.39), diazinon (O.1.40), dichlorvos/DDVP (O.1.41), dicrotophos (O.1.42), dimethoate (O.1.43), dimethylvinphos (O.1.44), disulfoton (O.1.45), EPN (O.1.46), ethion (O.1.47), ethoprophos (O.1.48), famphur (O.1.49), fenamiphos (O.1.50), fenitrothion (O.1.51), fenthion (O.1.52), fosthiazate (O.1.53), heptenophos (O.1.54), imicyafos (O.1.55), isofenphos (O.1.56), isopropyl O-(methoxyaminothio-phosphoryl) salicylate (O.1.57), isoxathion (O.1.58), malathion (O.1.59), mecarbam (O.1.60), methamidophos (O.1.61), methidathion (O.1.62), mevinphos (O.1.63), monocrotophos (O.1.64), naled (O.1.65), omethoate (O.1.66), oxydemeton-methyl (O.1.67), parathion (O.1.68), parathion-methyl (O.1.69), phenthoate (O.1.70), phorate (O.1.71), phosalone (O.1.72), phosmet (O.1.73), phosphamidon (O.1.74), phoxim (O.1.75), pirimiphos-methyl (O.1.76), profenofos (O.1.77), propetamphos (O.1.78), prothiofos (O.1.79), pyraclofos (O.1.80), pyridaphenthion (O.1.81), quinalphos (O.1.82), sulfotep (O.1.83), tebupirimfos (O.1.84), temephos (O.1.85), terbufos (O.1.86), tetrachlorvinphos (O.1.87), thiometon (O.1.88), triazophos (O.1.89), trichlorfon (O.1.90), vamidothion (O.1.91);
O.2 GABA-gated chloride channel antagonists: endosulfan (O.2.1), chlordane (O.2.2);
ethiprole (O.2.3), fipronil (O.2.4), flufiprole (O.2.5), pyrafluprole (O.2.6), pyriprole (O.2.7);
O.3 Sodium channel modulators: acrinathrin (O.3.1), allethrin (O.3.2), d-cis-trans allethrin (O.3.3), d-trans allethrin (O.3.4), bifenthrin (O.3.5), bioallethrin (O.3.6), bioallethrin S-cylclopentenyl (O.3.7), bioresmethrin (O.3.8), cycloprothrin (O.3.9), cyfluthrin (O.3.10), beta-cyfluthrin (O.3.11), cyhalothrin (O.3.12), lambda-cyhalothrin (O.3.13), gamma-cyhalothrin (O.3.14), cypermethrin (O.3.15), alpha-cypermethrin (O.3.16), beta-cypermethrin (O.3.17), theta-cypermethrin (O.3.18), zeta-cypermethrin (O.3.19), cyphenothrin (O.3.20), deltamethrin (O.3.21), empenthrin (O.3.22), esfenvalerate (O.3.23), etofenprox (O.3.24), fenpropathrin (O.3.25), fenvalerate (O.3.26), flucythrinate (O.3.27), flumethrin (O.3.28), tau-fluvalinate (O.3.29), halfenprox (O.3.30), heptafluthrin (O.3.31), imiprothrin (O.3.32), meperfluthrin (O.3.33), metofluthrin (O.3.34), momfluorothrin (O.3.35), permethrin (O.3.36), phenothrin (O.3.37), prallethrin (O.3.38), profluthrin (O.3.39), pyrethrin (pyrethrum) (O.3.40), resmethrin (O.3.41), silafluofen (O.3.42), tefluthrin (O.3.43), tetramethylfluthrin (O.3.44), tetramethrin (O.3.45), tralomethrin (O.3.46) and transfluthrin (O.3.47); DDT (O.3.48), methoxychlor (O.3.49);
O.4 Nicotinic acetylcholine receptor agonists (nAChR): acetamiprid (O.4.1), clothianidin (O.4.2), cycloxaprid (O.4.3), dinotefuran (O.4.4), imidacloprid (O.4.5), nitenpyram (O.4.6), thiacloprid (O.4.7), thiamethoxam (O.4.8); (2E)-1-[(6-chloropyridin-3-yl)methyl]-N′-nitro-2-pentylidenehydrazinecarboximidamide (O.4.9); 1-[(6-chloropyridin-3-yl)methyl]-7-methyl-8-nitro-5-propoxy-1,2,3,5,6,7-hexahydroimidazo[1,2-a]pyridine (O.4.10); nicotine (O.4.11);
O.5 Nicotinic acetylcholine receptor allosteric activators: spinosad (O.5.1), spinetoram (O.5.2);
O.6 Chloride channel activators: abamectin (O.6.1), emamectin benzoate (O.6.2), ivermectin (O.6.3), lepimectin (O.6.4), milbemectin (O.6.5);
O.7 Juvenile hormone mimics: hydroprene (O.7.1), kinoprene (O.7.2), methoprene (O.7.3); fenoxycarb (O.7.4), pyriproxyfen (O.7.5);
O.8 miscellaneous non-specific (multi-site) inhibitors: methyl bromide (O.8.1) and other alkyl halides; chloropicrin (O.8.2), sulfuryl fluoride (O.8.3), borax (O.8.4), tartar emetic (O.8.5);
O.9 Selective homopteran feeding blockers: pymetrozine (O.9.1), flonicamid (O.9.2);
O.10 Mite growth inhibitors: clofentezine (O.10.1), hexythiazox (O.10.2), diflovidazin (O.10.3); etoxazole (O.10.4);
O.11 Microbial disruptors of insect midgut membranes: the Bt crop proteins: Cry1Ab, Cry1Ac, Cry1Fa, Cry2Ab, mCry3A, Cry3Ab, Cry3Bb, Cry34/35Ab1;
O.12 Inhibitors of mitochondrial ATP synthase: diafenthiuron (O.12.1); azocyclotin (O.12.2), cyhexatin (O.12.3), fenbutatin oxide (O.12.4), propargite (O.12.5), tetradifon (O.12.6);
O.13 Uncouplers of oxidative phosphorylation via disruption of the proton gradient: chlorfenapyr (O.13.1), DNOC (O.13.2), sulfluramid (O.13.3);
O.14 Nicotinic acetylcholine receptor (nAChR) channel blockers: bensultap (O.14.1), cartap hydrochloride (O.14.2), thiocyclam (O.14.3), thiosultap sodium (O.14.4);
O.15 Inhibitors of the chitin biosynthesis type 0: bistrifluron (O.15.1), chlorfluazuron (O.15.2), diflubenzuron (O.15.3), flucycloxuron (O.15.4), flufenoxuron (O.15.5), hexaflumuron (O.15.6), lufenuron (O.15.7), novaluron (O.15.8), noviflumuron (O.15.9), teflubenzuron (O.15.10), triflumuron (O.15.11);
O.16 Inhibitors of the chitin biosynthesis type 1: buprofezin (O.16.1);
O.17 Moulting disruptors: cyromazine (O.17.1);
O.18 Ecdyson receptor agonists: methoxyfenozide (O.18.1), tebufenozide (O.18.2), halofenozide (O.18.3), fufenozide (O.18.4), chromafenozide (O.18.5);
O.19 Octopamin receptor agonists: amitraz (O.19.1);
O.20 Mitochondrial complex III electron transport inhibitors: hydramethylnon (O.20.1), acequinocyl (O.20.2), fluacrypyrim (O.20.3);
O.21 Mitochondrial complex I electron transport inhibitors: fenazaquin (O.21.1), fenpyroximate (O.21.2), pyrimidifen (O.21.3), pyridaben (O.21.4), tebufenpyrad (O.21.5), tolfenpyrad (O.21.6); rotenone (O.21.7);
O.22 Voltage-dependent sodium channel blockers: indoxacarb (O.22.1), metaflumizone (O.22.2), 2-[2-(4-cyanophenyl)-1-[3-(trifluoromethyl)phenyl]-ethylidene]-N-[4-(difluoromethoxy)phenyl]-hydrazinecarboxamide (O.22.3), N-(3-chloro-2-methylphenyl)-2-[(4-chlorophenyl)-[4-[methyl(methylsulfonyl)-amino]phenyl]methylene]-hydrazinecarboxamide (O.22.4);
O.23 Inhibitors of the of acetyl CoA carboxylase: spirodiclofen (O.23.1), spiromesifen (O.23.2), spirotetramat (O.23.3);
O.24 Mitochondrial complex IV electron transport inhibitors: aluminium phosphide (O.24.1), calcium phosphide (O.24.2), phosphine (O.24.3), zinc phosphide (O.24.4), cyanide (O.24.5);
O.25 Mitochondrial complex II electron transport inhibitors: cyenopyrafen (O.25.1), cyflumetofen (O.25.2);
O.26 Ryanodine receptor-modulators: flubendiamide (O.26.1), chlorantraniliprole (O.26.2), cyantraniliprole (O.26.3), cyclaniliprole (O.26.4), tetraniliprole (O.26.5); (R)-3-chloro-N1-{2-methyl-4-[1,2,2,2-tetrafluoro-1-(trifluoromethyl)ethyl]phenyl}-N2-(1-methyl-2-methylsulfonylethyl)phthalamide (O.26.6), (S)-3-chloro-N1-{2-methyl-4-[1,2,2,2-tetrafluoro-1-(trifluoromethyl)ethyl]phenyl}-N2-(1-methyl-2-methylsulfonylethyl)phthalamide (O.26.7), methyl-2-[3,5-dibromo-2-({[3-bromo-1-(3-chloropyridin-2-yl)-1H-pyrazol-5-yl]carbonyl}amino)benzoyl]-1,2-dimethylhydrazinecarboxylate (O.26.8); N-[4,6-dichloro-2-[(diethyl-lambda-4-sulfanylidene)carbamoyl]-phenyl]-2-(3-chloro-2-pyridyl)-5-(trifluoromethyl)pyrazole-3-carboxamide (O.26.9); N-[4-chloro-2-[(diethyl-lambda-4-sulfanylidene)carbamoyl]-6-methyl-phenyl]-2-(3-chloro-2-pyridyl)-5-(trifluoromethyl)pyrazole-3-carboxamide (O.26.10); N-[4-chloro-2-[(di-2-propyl-lambda-4-sulfanylidene)carbamoyl]-6-methyl-phenyl]-2-(3-chloro-2-pyridyl)-5-(trifluoromethyl)pyrazole-3-carboxamide (O.26.11); N-[4,6-dichloro-2-[(di-2-propyl-lambda-4-sulfanylidene)carbamoyl]-phenyl]-2-(3-chloro-2-pyridyl)-5-(trifluoromethyl)pyrazole-3-carboxamide (O.26.12); N-[4,6-di-bromo-2-[(diethyl-lambda-4-sulfanylidene)carbamoyl]-phenyl]-2-(3-chloro-2-pyridyl)-5-(trifluoromethyl)pyrazole-3-carboxamide (O.26.13); N-[2-(5-amino-1,3,4-thiadiazol-2-yl)-4-chloro-6-methylphenyl]-3-bromo-1-(3-chloro-2-pyridinyl)-1H-pyrazole-5-carboxamide (O.26.14); 3-chloro-1-(3-chloro-2-pyridinyl)-N-[2,4-dichloro-6-[[(1-cyano-1-methylethyl)amino]carbonyl]phenyl]-1H-pyrazole-5-carboxamide (O.26.15); 3-bromo-N-[2,4-dichloro-6-(methylcarbamoyl)phenyl]-1-(3,5-dichloro-2-pyridyl)-1H-pyrazole-5-carboxamide (O.26.16); N-[4-chloro-2-[[(1,1-dimethylethyl)amino]carbonyl]-6-methylphenyl]-1-(3-chloro-2-pyridinyl)-3-(fluoromethoxy)-1H-pyrazole-5-carboxamide (O.26.17); cyhalodiamide (O.26.18);
O.27 insecticidal active compounds of unknown or uncertain mode of action: afidopyropen (O.27.1), afoxolaner (O.27.2), azadirachtin (O.27.3), amidoflumet (O.27.4), benzoximate (O.27.5), bifenazate (O.27.6), broflanilide (O.27.7), bromopropylate (O.27.8), chinomethionat (O.27.9), cryolite (O.27.10), dicloromezotiaz (O.27.11), dicofol (O.27.12), flufenerim (O.27.13), flometoquin (O.27.14), fluensulfone (O.27.15), fluhexafon (O.27.16), fluopyram (O.27.17), flupyradifurone (O.27.18), fluralaner (O.27.19), metoxadiazone (O.27.20), piperonyl butoxide (O.27.21), pyflubumide (O.27.22), pyridalyl (O.27.23), pyrifluquinazon (O.27.24), sulfoxaflor (O.27.25), tioxazafen (O.27.26), triflumezopyrim (O.27.27), 11-(4-chloro-2,6-dimethylphenyl)-12-hydroxy-1,4-dioxa-9-azadispiro[4.2.4.2]-tetradec-11-en-10-one (O.27.28), 3-(4′-fluoro-2,4-dimethylbiphenyl-3-yl)-4-hydroxy-8-oxa-1-azaspiro[4.5]dec-3-en-2-one (O.27.28), 1-[2-fluoro-4-methyl-5-[(2,2,2-trifluoroethyl)sulfinyl]phenyl]-3-(trifluoromethyl)-1H-1,2,4-triazole-5-amine (O.27.29), (E/Z)—N-[1-[(6-chloro-3-pyridyl)methyl]-2-pyridylidene]-2,2,2-trifluoro-acetamide (O.27.31); (E/Z)—N-[1-[(6-chloro-5-fluoro-3-pyridyl)methyl]-2-pyridylidene]-2,2,2-trifluoro-acetamide (O.27.32); (E/Z)-2,2,2-trifluoro-N-[1-[(6-fluoro-3-pyridyl)methyl]-2-pyridylidene]acetamide (O.27.33); (E/Z)—N-[1-[(6-bromo-3-pyridyl)methyl]-2-pyridylidene]-2,2,2-trifluoro-acetamide (O.27.34); (E/Z)—N-[1-[1-(6-chloro-3-pyridyl)ethyl]-2-pyridylidene]-2,2,2-trifluoro-acetamide (O.27.35); (E/Z)—N-[1-[(6-chloro-3-pyridyl)methyl]-2-pyridylidene]-2,2-difluoro-acetamide (O.27.36); (E/Z)-2-chloro-N-[1-[(6-chloro-3-pyridyl)methyl]-2-pyridylidene]-2,2-difluoro-acetamide (O.27.37); (E/Z)—N-[1-[(2-chloropyrimidin-5-yl)methyl]-2-pyridylidene]-2,2,2-trifluoro-acetamide (O.27.38); (E/Z)—N-[1-[(6-chloro-3-pyridyl)methyl]-2-pyridylidene]-2,2,3,3,3-pentafluoro-propanamide (O.27.39); N-[1-[(6-chloro-3-pyridyl)methyl]-2-pyridylidene]-2,2,2-trifluoro-thioacetamide (O.27.40); N-[1-[(6-chloro-3-pyridyl)methyl]-2-pyridylidene]-2,2,2-trifluoro-N′-isopropyl-acetamidine (O.27.41); fluazaindolizine (O.27.42); 4-[5-(3,5-dichlorophenyl)-5-(trifluoromethyl)-4H-isoxazol-3-yl]-2-methyl-N-(1-oxothietan-3-yl)benzamide (O.27.43); fluxametamide (O.27.44); 5-[3-[2,6-dichloro-4-(3,3-dichloroallyloxy)phenoxy]propoxy]-1H-pyrazole (O.27.45); 3-(benzoylmethylamino)-N-[2-bromo-4-[1,2,2,3,3,3-hexafluoro-1-(trifluoromethyl)propyl]-6-(trifluoromethyl)phenyl]-2-fluoro-benzamide (O.27.46); 3-(benzoylmethylamino)-2-fluoro-N-[2-iodo-4-[1,2,2,2-tetrafluoro-1-(trifluoromethyl)ethyl]-6-(trifluoromethyl)phenyl]-benzamide (O.27.47); N-[3-[[[2-iodo-4-[1,2,2,2-tetrafluoro-1-(trifluoromethyl)ethyl]-6-(trifluoromethyl)phenyl]amino]carbonyl]phenyl]-N-methyl-benzamide (O.27.48); N-[3-[[[2-bromo-4-[1,2,2,2-tetrafluoro-1-(trifluoromethyl)ethyl]-6-(trifluoromethyl)phenyl]amino]carbonyl]-2-fluorophenyl]-4-fluoro-N-methyl-benzamide (O.27.49); 4-fluoro-N-[2-fluoro-3-[[[2-iodo-4-[1,2,2,2-tetrafluoro-1-(trifluoromethyl)ethyl]-6-(trifluoromethyl)phenyl]amino]carbonyl]phenyl]-N-methyl-benzamide (O.27.50); 3-fluoro-N-[2-fluoro-3-[[[2-iodo-4-[1,2,2,2-tetrafluoro-1-(trifluoromethyl)ethyl]-6(trifluoromethyl)phenyl]amino]carbonyl]phenyl]-N-methyl-benzamide (O.27.51); 2-chloro-N-[3-[[[2-iodo-4-[1,2,2,2-tetrafluoro-1-(tri-fluoromethyl)ethyl]-6-(trifluoromethyl)phenyl]amino]carbonyl]phenyl]-3-pyridine-carboxamide (O.27.52); 4-cyano-N-[2-cyano-5-[[2,6-dibromo-4-[1,2,2,3,3,3-hexafluoro-1-(trifluoromethyl)propyl]phenyl]carbamoyl]phenyl]-2-methyl-benzamide (O.27.53); 4-cyano-3-[(4-cyano-2-methyl-benzoyl)amino]-N-[2,6-dichloro-4-[1,2,2,3,3,3-hexafluoro-1-(trifluoromethyl)propyl]phenyl]-2-fluoro-benzamide (O.27.54); N-[5-[[2-chloro-6-cyano-4-[1,2,2,3,3,3-hexafluoro-1-(trifluoromethyl)propyl]phenyl]carbamoyl]-2-cyano-phenyl]-4-cyano-2-methyl-benzamide (O.27.55); N-[5-[[2-bromo-6-chloro-4-[2,2,2-trifluoro-1-hydroxy-1-(trifluoromethyl)ethyl]phenyl]carbamoyl]-2-cyano-phenyl]-4-cyano-2-methyl-benzamide (O.27.56); N-[5-[[2-bromo-6-chloro-4-[1,2,2,3,3,3-hexafluoro-1-(trifluoro-methyl)propyl]phenyl]carbamoyl]-2-cyano-phenyl]-4-cyano-2-methyl-benzamide (O.27.57); 4-cyano-N-[2-cyano-5-[[2,6-dichloro-4-[1,2,2,3,3,3-hexafluoro-1-(trifluoromethyl)propyl]phenyl]carbamoyl]phenyl]-2-methyl-benzamide (O.27.58); 4-cyano-N-[2-cyano-5-[[2,6-dichloro-4-[1,2,2,2-tetrafluoro-1-(trifluoromethyl)ethyl]phenyl]carbamoyl]phenyl]-2-methyl-benzamide (O.27.59); N-[5-[[2-bromo-6-chloro-4-[1,2,2,2-tetrafluoro-1-(trifluoromethyl)ethyl]phenyl]carbamoyl]-2-cyano-phenyl]-4-cyano-2-methyl-benzamide (O.27.60); 2-(1,3-dioxan-2-yl)-6-[2-(3-pyridinyl)-5-thiazolyl]-pyridine; 2-[6-[2-(5-fluoro-3-pyridinyl)-5-thiazolyl]-2-pyridinyl]-pyrimidine (O.27.61); 2-[6-[2-(3-pyridinyl)-5-thiazolyl]-2-pyridinyl]-pyrimidine (O.27.62); N-methylsulfonyl-6-[2-(3-pyridyl)thiazol-5-yl]pyridine-2-carboxamide (O.27.63); N-methylsulfonyl-6-[2-(3-pyridyl)thiazol-5-yl]pyridine-2-carboxamide (O.27.64); N-ethyl-N-[4-methyl-2-(3-pyridyl)thiazol-5-yl]-3-methylthio-propanamide (O.27.65); N-methyl-N-[4-methyl-2-(3-pyridyl)thiazol-5-yl]-3-methylthio-propanamide (O.27.66); N,2-dimethyl-N-[4-methyl-2-(3-pyridyl)thiazol-5-yl]-3-methylthio-propanamide (O.27.67); N-ethyl-2-methyl-N-[4-methyl-2-(3-pyridyl)thiazol-5-yl]-3-methylthio-propanamide (O.27.68); N-[4-chloro-2-(3-pyridyl)thiazol-5-yl]-N-ethyl-2-methyl-3-methylthio-propanamide (O.2769.); N-[4-chloro-2-(3-pyridyl)thiazol-5-yl]-N,2-dimethyl-3-methylthio-propanamide (O.27.70); N-[4-chloro-2-(3-pyridyl)thiazol-5-yl]-N-methyl-3-methylthio-propanamide (O.27.71); N-[4-chloro-2-(3-pyridyl)thiazol-5-yl]-N-ethyl-3-methylthio-propanamide (O.27.72); 1-[(6-chloro-3-pyridinyl)methyl]-1,2,3,5,6,7-hexahydro-5-methoxy-7-methyl-8-nitro-imidazo[1,2-a]pyridine (O.27.73); 1-[(6-chloropyridin-3-yl)methyl]-7-methyl-8-nitro-1,2,3,5,6,7-hexahydroimidazo[1,2-a]pyridin-5-ol (O.27.74); 1-isopropyl-N,5-dimethyl-N-pyridazin-4-yl-pyrazole-4-carboxamide (O.27.75); 1-(1,2-dimethylpropyl)-N-ethyl-5-methyl-N-pyridazin-4-yl-pyrazole-4-carboxamide (O.27.76); N,5-dimethyl-N-pyridazin-4-y-1-(2,2,2-trifluoro-1-methyl-ethyl)pyrazole-4-carboxamide (O.27.77); 1-[1-(1-cyanocyclopropyl)ethyl]-N-ethyl-5-methyl-N-pyridazin-4-yl-pyrazole-4-carboxamide (O.27.78); N-ethyl-1-(2-fluoro-1-methyl-propyl)-5-methyl-N-pyridazin-4-yl-pyrazole-4-carboxamide (O.27.79); 1-(1,2-dimethylpropyl)-N,5-dimethyl-N-pyridazin-4-yl-pyrazole-4-carboxamide (O.27.80); 1-[1-(1-cyanocyclopropyl)ethyl]-N,5-dimethyl-N-pyridazin-4-yl-pyrazole-4-carboxamide (O.27.81); N-methyl--(2-fluoro-1-methyl-propyl]-5-methyl-N-pyridazin-4-yl-pyrazole-4-carboxamide (O.27.82); 1-(4,4-difluorocyclohexyl)-N-ethyl-5-methyl-N-pyridazin-4-yl-pyrazole-4-carboxamide (O.27.83); 1-(4,4-difluorocyclohexyl)-N,5-dimethyl-N-pyridazin-4-yl-pyrazole-4-carboxamide (O.27.84), N-(1-methylethyl)-2-(3-pyridinyl)-2H-indazole-4-carboxamide (O.27.85); N-cyclopropyl-2-(3-pyridinyl)-2H-indazole-4-carboxamide (O.27.86); N-cyclohexyl-2-(3-pyridinyl)-2H-indazole-4-carboxamide (O.27.87); 2-(3-pyridinyl)-N-(2,2,2-trifluoroethyl)-2H-indazole-4-carboxamide (O.27.88); 2-(3-pyridinyl)-N-[(tetrahydro-2-furanyl)methyl]-2H-indazole-5-carboxamide (O.27.89); methyl 2-[[2-(3-pyridinyl)-2H-indazol-5-yl]carbonyl]hydrazinecarboxylate (O.27.90); N-[(2,2-difluorocyclopropyl)methyl]-2-(3-pyridinyl)-2H-indazole-5-carboxamide (O.27.91); N-(2,2-difluoropropyl)-2-(3-pyridinyl)-2H-indazole-5-carboxamide (O.27.92); 2-(3-pyridinyl)-N-(2-pyrimidinylmethyl)-2H-indazole-5-carboxamide (O.27.93); N-[(5-methyl-2-pyrazinyl)methyl]-2-(3-pyridinyl)-2H-indazole-5-car-boxamide (O.27.94), N-[3-chloro-1-(3-pyridyl)pyrazol-4-yl]-N-ethyl-3-(3,3,3-trifluoropropylsulfanyl)propanamide (O.27.95); N-[3-chloro-1-(3-pyridyl)pyrazol-4-yl]-N-ethyl-3-(3,3,3-trifluoropropylsulfinyl)propanamide (O.27.96); N-[3-chloro-1-(3-pyridyl)pyrazol-4-yl]-3-[(2,2-difluorocyclopropyl)methylsulfanyl]-N-ethyl-propanamide (O.27.97); N-[3-chloro-1-(3-pyridyl)pyrazol-4-yl]-3-[(2,2-difluorocyclopropyl)methylsulfinyl]-N-ethyl-propanamide (O.27.98); sarolaner (O.27.99), lotilaner (O.27.100);
in a weight ratio of from 100:1 to 1:100.

12: A fungicidal mixture comprising as active components
1) at least one active compound I as defined in claim 11, or an N-oxide, or an agriculturally useful salt thereof;
and as component
2) at least one active compound II selected from groups A) to 0) as defined for component 2) in claim 11, or an N-oxide, or an agriculturally useful salt thereof;
and as component
3) at least one active compound III selected from groups A) to O) as defined for component 2) in claim 11, or an N-oxide, or an agriculturally useful salt thereof;
wherein the at least one active compound III of component 3) is not identical with the at least one active compound II of component 2).

13: The mixture according to claim 12, comprising a compound I and a compound II and a further active compound III in a synergistically effective amount.

14: The mixture according to claim 12, comprising a compound I and a compound II in a weight ratio of from 100:1 to 1:100, and a compound I and compound III in a weight ratio of from 100:1 to 1:100.

15: The mixture according to claim 11, wherein R² in compounds of the formula I is hydrogen, C₁-C₆-alkyl, C₂-C₆-alkenyl, C₂-C₆-alkynyl, C₁-C₆-alkoxy or C₃-C₈-cycloalkyl.

16: The mixture according to claim 11, wherein the component 1) is at least one active compound I selected from the group consisting of
N-methyl-4-[5-(trifluoromethyl)-1,2,4-oxadiazol-3-yl]benzamide,
N—[(E)-methoxyiminomethyl]-4-[5-(trifluoromethyl)-1,2,4-oxadiazol-3-yl]benzamide,
N—[(Z)-methoxyiminomethyl]-4-[5-(trifluoromethyl)-1,2,4-oxadiazol-3-yl]benzamide,
N-[4-[5-(trifluoromethyl)-1,2,4-oxadiazol-3-yl]phenyl]cyclopropanecarboxamide,
N-(2-fluorophenyl)-4-[5-(trifluoromethyl)-1,2,4-oxadiazol-3-yl]benzamide,
2,2-difluoro-N-methyl-2-[4-[5-(trifluoromethyl)-1,2,4-oxadiazol-3-yl]phenyl]acetamide,
N-allyl-N-[[4-[5-(trifluoromethyl)-1,2,4-oxadiazol-3-yl]phenyl]methyl]acetamide,
N—[(E)-N-methoxy-C-methyl-carbonimidoyl]-4-[5-(trifluoromethyl)-1,2,4-oxadiazol-3-yl]benzamide,
N—[(Z)—N-methoxy-C-methyl-carbonimidoyl]-4-[5-(trifluoromethyl)-1,2,4-oxadiazol-3-yl]benzamide,
N-allyl-N-[[4-[5-(trifluoromethyl)-1,2,4-oxadiazol-3-yl]phenyl]methyl]propanamide,
4,4-dimethyl-1-[[4-[5-(trifluoromethyl)-1,2,4-oxadiazol-3-yl]phenyl]methyl]pyrrolidin-2-one,
N-methyl-4-[5-(trifluoromethyl)-1,2,4-oxadiazol-3-yl]benzenecarbothioamide or 5-methyl-1-[[4-[5-(trifluoromethyl)-1,2,4-oxadiazol-3-yl]phenyl]methyl]pyrrolidin-2-one.

17: An agrochemical composition, comprising a solvent or solid carrier and a composition according to claim 11.

18: The agrochemical composition according to claim 17 further comprising seed in an amount of from 1 g to 1000 g active components per 100 kg of seed.

19: A method for controlling phytopathogenic harmful fungi, comprising treating the fungi, their habitat or the seed, the soil or the plants to be protected against fungal attack with an effective amount of the mixture of claim 11.

20: A method for controlling phytopathogenic harmful fungi, comprising treating the fungi, their habitat or the seed, the soil or the plants to be protected against fungal attack with an effective amount of the composition of claim 17.

21: The method of claim 19, wherein the mixture comprises as active components
1) at least one active compound I as defined in claim 11, or an N-oxide, or an agriculturally useful salt thereof;
and as component
2) at least one active compound II selected from groups A) to O), or an N-oxide, or an agriculturally useful salt thereof;
and as component
3) at least one active compound III selected from groups A) to O), or an N-oxide, or an agriculturally useful salt thereof;
wherein the at least one active compound III of component 3) is not identical with the at least one active compound II of component 2).

22: The method of claim 19, wherein the mixture comprises a compound I and a compound II and a further active compound III in a synergistically effective amount.

23: The method of claim 19, wherein the mixture comprises a compound I and a compound II in a weight ratio of from 100:1 to 1:100, and a compound I and compound III in a weight ratio of from 100:1 to 1:100.

24: The method of claim 19, wherein R² in compounds of the formula I is hydrogen, C₁-C₆-alkyl, C₂-C₆-alkenyl, C₂-C₆-alkynyl, C₁-C₆-alkoxy or C₃-C₈-cycloalkyl.

25: The method of claim 19, wherein the component 1) is at least one active compound I selected from the group consisting of
N-methyl-4-[5-(trifluoromethyl)-1,2,4-oxadiazol-3-yl]benzamide,
N—[(E)-methoxyiminomethyl]-4-[5-(trifluoromethyl)-1,2,4-oxadiazol-3-yl]benzamide,
N—[(Z)-methoxyiminomethyl]-4-[5-(trifluoromethyl)-1,2,4-oxadiazol-3-yl]benzamide,
N-[4-[5-(trifluoromethyl)-1,2,4-oxadiazol-3-yl]phenyl]cyclopropanecarboxamide,
N-(2-fluorophenyl)-4-[5-(trifluoromethyl)-1,2,4-oxadiazol-3-yl]benzamide,
2,2-difluoro-N-methyl-2-[4-[5-(trifluoromethyl)-1,2,4-oxadiazol-3-yl]phenyl]acetamide,
N-allyl-N-[[4-[5-(trifluoromethyl)-1,2,4-oxadiazol-3-yl]phenyl]methyl]acetamide,
N—[(E)-N-methoxy-C-methyl-carbonimidoyl]-4-[5-(trifluoromethyl)-1,2,4-oxadiazol-3-yl]benzamide,
N—[(Z)—N-methoxy-C-methyl-carbonimidoyl]-4-[5-(trifluoromethyl)-1,2,4-oxadiazol-3-yl]benzamide,
N-allyl-N-[[4-[5-(trifluoromethyl)-1,2,4-oxadiazol-3-yl]phenyl]methyl]propanamide, 4,4-dimethyl-1-[[4-[5-(trifluoromethyl)-1,2,4-oxadiazol-3-yl]phenyl]methyl]pyrrolidin-2-one,
N-methyl-4-[5-(trifluoromethyl)-1,2,4-oxadiazol-3-yl]benzenecarbothioamide or 5-methyl-1-[[4-[5-(trifluoromethyl)-1,2,4-oxadiazol-3-yl]phenyl]methyl]pyrrolidin-2-one.
